(19) United States
(12) Patent Application Publication
(10) Pub. No.: US 2026/0273097 A1
(43) Pub. Date: Sep. 17, 2026

(54) COMPOSITIONS AND METHODS FOR THE TREATMENT OF DISORDERS RELATED TO SYNTAXIN-BINDING PROTEIN 1 DEFICIENCY

(71) Applicant: VOYAGER THERAPEUTICS, INC., Lexington, MA (US)

(72) Inventors: Mathieu Emmanuel NONNENMACHER, Lexington, MA (US); Tyler Christopher MOYER, Lexington, MA (US); Jiangyu LI, Lexington, MA (US); Dan Richard LAKS, Lexington, MA (US)

(21) Appl. No.: 19/167,950

(22) PCT Filed: May 1, 2024
(86) PCT No.: PCT/US2024/027318

§ 371 Date: Sep. 23, 2025

Related U.S. Application Data
(60) Provisional application No. 63/499,925, filed on May 3, 2023.
(60) Provisional application No. 63/593,828, filed on Oct. 27, 2023.
(60) Provisional application No. 63/564,420, filed on Mar. 12, 2024.

(51) Int. Cl. A61K 48/00; A61K 31/436; A61K 31/5377; A61K 31/573; A61K 38/00; A61K 39/395; C07K 14/47; C12N 15/86

(52) U.S. Cl. CPC A61K 48/0033; A61K 31/436; A61K 31/5377; A61K 31/573; A61K 39/3955; A61K 48/005; C07K 14/47; C12N 15/86; A61K 38/00; C12N 2750/14122; C12N 2750/14143; C12N 2750/14151

(57) ABSTRACT

The disclosure relates to compositions and methods for altering, e.g., enhancing, the expression of STXBP1 proteins via delivery using an adeno-associated virus (AAV) capsid variant. The compositions and methods of the present disclosure are useful in the treatment of subjects diagnosed with, or suspected of having STXBP1 encephalopathy, or another STXBP1-related disorder.

### RELATED APPLICATIONS

This application claims the benefit of and priority to US Provisional Application Ser. No. 63/499,925, filed May 3, 2023, US Provisional Application Ser. No. 63/593,828, filed Oct. 27, 2023, and U.S. Provisional Application Ser. No. 63/564,420, filed Mar. 12, 2024, the contents of each of which are incorporated herein by reference in their entirety.

### SEQUENCE LISTING

The present application is being filed along with a Sequence Listing in electronic format. The Sequence Listing file, entitled 14640_0085-00304_SL.xml, was created on Mar. 26, 2024, and is 5,536,190 bytes in size. The information in electronic format of the Sequence Listing is incorporated herein by reference in its entirety.

### FIELD

Described herein are compositions and methods relating to adeno-associated virus (AAV) viral particles for the delivery of polynucleotides, e.g., polynucleotides encoding syntaxin-binding protein-1 (STXBP1) proteins (“STXBP1” protein”) and peptides (“STXBP1 peptide”) for use in the treatment of STXBP1 encephalopathy, and other STXBP1-related disorders, including epileptic encephalopathy, Ohtahara syndrome, developmental encephalopathy, West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), and Rett syndrome phenotype (not caused by mutation of MECP2 or CDKL5) (collectively. “STXBP1-related disorders”). In some embodiments, compositions described herein may be used to treat a subject in need thereof, such as a human subject diagnosed with a STXBP1-related disorder or other condition resulting from a deficiency in the quantity and/or function of STXBP1 protein.

### BACKGROUND

Syntaxin-binding protein 1 (STXBP1), also known as Munc18-1, is part of the synaptic fusion machinery that enables vesicles to fuse with the plasma membrane. Other names for STXBP1 include P67, DEE4, NSEC1, UNC18, N-Sec1, RBSEC1, unc-18A, and unc18-1, STXBP1 regulates neurotransmitter transmission by interacting with the SNARE complex. The SNARE complex is primarily composed of SNAP-25, vesicular associated membrane protein and syntaxin1, SNAP-25 and syntaxin-1 form the target membrane vesicle protein (T-SNARE), which binds to synaptic vesicle protein (VAMP), STXBP1 has a complex, arched tertiary structure. The arch comprises four closely connected domains, 1, 2, 3a and 3b. Domains 1 and 3a form an arched gap. STXBP1 primarily regulates vesicle fusion by interacting with syntaxin-1. Domain 3a of STXBP1 is in close contact with the Habc domain of syntaxin-1, and domain 1 of STXBP1 binds to the N-terminal domain of syntaxin-1, STXBP1 regulates vesicle docking and fusion by interacting with the SNARE complex. STXBP1 affects the released vesicles, and participates in the transmission of neurotransmitters. STXBP1 is prominently involved in the early process of neurotransmitter release.

STXBP1 is essential for presynaptic vesicle release. It is rapidly phosphorylated by protein kinase C upon neuronal depolarization.

STXBP1 is encoded by the STXBP1 gene (Ensembl Gene ID No. ENSG0000136854), which is located on chromosome 9. It is expressed in the brain and spinal cord, and highly enriched in axons. Expression of STXBP1 is highest in the retina and cerebellum. STXBP1 is also found outside the brain.

Mutations in the STXBP1 gene are known to cause disease in human subjects. Abnormal expression of STXBP1 plays a role in the pathogenesis of a variety of neurological diseases, including STXBP1 encephalopathy, epileptic encephalopathy, Ohtahara syndrome, developmental encephalopathy. West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), and Rett syndrome phenotype (not caused by mutation of MECP2 or CDKL5).

STXBP1 expression is also abnormal in STXBP1 encephalopathy. There are an estimated 750 known cases of STXBP1 encephalopathy worldwide, and has an estimated incidence of 3.3-3.8 per 100,000 births.

Patients with STXBP1 mutations and/or STXBP1 encephalopathy often present with epilepsy. Some patients with STXBP1 mutations present with autistic features, including aggressive behavior, self-mutilation, hyperactivity, compulsive symptoms, episodes of psychosis and/or auditory hallucinations. Phenotypes range from severe neonatal epilepsy to infantile-onset epilepsy.

Many patients with STXBP1 mutations also present with non-epileptic movement disorders, including truncal and limb ataxia, generalized tremors, and dystonia. Patients often present with unremitting epileptic activity.

Patients with STXBP1 encephalopathy are reliant on caregivers for the duration of their lives. Moreover, 40% of patients become non-ambulatory and lifespan is expected to be significantly reduced to about 30 years.

STXBP1 encephalopathy is caused by haploinsufficiency. Thus, disease may occur where there is a mutation to only a single functional copy of the STXBP1 gene. Disease-causing mutations include missense, nonsense, frameshift, splice-site, and whole gene deletions. There are about 135 known pathogenic variants of the STXBP1 gene.

Interneurons may be more affected by haploinsufficiency than excitatory neurons.

Studies have demonstrated that heterozygous STXBP1 knock-out mice display impaired glutamate and GABA transmission, increased anxiety, increased aggression, and impaired emotional learning, in addition to modest seizure phenotype.

Studies in mice demonstrated that normalizing the excitatory synaptic transmission in STXBP1 heterozygotic knockout mice reduces aggression. This indicated a therapeutic option for managing aggressiveness in patients with STXBP1 mutations.

Existing therapies target the symptoms of STXBP1 encephalopathy. Existing first line treatment comprises anti-epileptic drugs such as levetiracetam and phenobarbital. Existing second line treatment comprises further anti-epileptic drugs such as clobazam, topiramate. Existing third line treatment comprises further anti-epileptic drugs and/or interventions. Known interventions for existing third line treatment include adrenocorticotropic hormone, ketogenic diet and vagal nerve stimulation.

No approved therapies target the underlying cause of STXBP1 mutations. There is a high unmet need for such treatments driven by the high seizure rate and shortened lifespan of patients with STXBP1 mutations.

Thus, there remains a long-felt need to develop pharmaceutical compositions and methods that can be delivered to the CNS for the treatment of diseases associated with STXBP1 mutations. In particular, a need exists for treatments targeting neurons, including GABAergic and glutamatergic neurons.

Adeno-associated viruses (AAVs) have emerged as a widely studied and utilized viral particles for delivery of therapeutically effective polypeptides to mammalian cells. See, e.g., Tratschin et al., Mol. Cell Biol., 5(11):3251-3260 (1985) and Grimm et al., Hum. Gene Ther., 10(15):2445-2450 (1999), the contents of each of which are incorporated herein by reference in their entirety.

There remains a need for effective methods of treatment using AAV capsid variants that are capable of delivering STXBP1 to a target cell or tissue, e.g., a CNS cell or tissue.

### SUMMARY

In some embodiments, the present disclosure provides an adeno-associated virus (AAV) particle comprising: a) an AAV capsid variant comprising an amino acid sequence having the following formula: [N1]-[N2]-[N3], wherein: (i) optionally [N1] comprises X1, X2, and X3, wherein at least one of X1, X2, or X3 is G; (ii) [N2] comprises the amino acid sequence of SPH; and (iii) [N3] comprises X4, X5, and X6, wherein at least one of X4, X5, or X6 is a basic amino acid, and b) a viral genome comprising a syntaxin-binding protein 1 (STXBP1)-encoding sequence. In some embodiments, the amino acid sequence [N1]-[N2]-[N3] is in hypervariable loop IV of the AAV capsid variant. In some embodiments, the AAV capsid variant is an AAV9 capsid variant. In some embodiments, [N1] comprises X1, X2, and X3, wherein at least one of X1, X2, or X3 is G, In some embodiments. [N2]-[N3] comprises the amino acid sequence of SPHSKA (SEQ ID NO: 941).

In some embodiments, the present disclosure provides an adeno-associated vims (AAV) particle comprising a viral genome comprising a syntaxin-binding protein 1 (STXBP1)-encoding sequence and an AAV9 capsid variant comprising the amino acid sequence of SPHSKA (SEQ ID NO: 941). In some embodiments, the amino acid sequence of SPHSKA (SEQ ID NO: 941) is in hypervariable loop IV of the AAV9 capsid variant. In some embodiments, the amino acid sequence of SPHSKA (SEQ ID NO: 941) is present immediately subsequent to an amino acid position corresponding to position 455 of SEQ ID NO: 4 or SEQ ID NO: 36.

In some embodiments, the AAV9 capsid variant comprises one, two, or all of: an N at an amino acid position corresponding to position 452, an E at an amino acid position corresponding to position 451, and/or a V at an amino acid position corresponding to position 453 of SEQ ID NO: 4. In some embodiments, the AAV9 capsid variant comprises the amino acid sequence of KTENVSGSPHSKAQNQQT (SEQ ID NO: 3272).

In some embodiments, the AAV9 capsid variant comprises: (i) a VP1 protein comprising an amino acid sequence having at least 90% identity to SEQ ID NO: 4; (ii) a VP2 protein comprising an amino acid sequence having at least 90% identity to positions 138-742 of SEQ ID NO: 4; and/or (iii) a VP3 protein comprising an amino acid sequence having at least 90% identity to positions 203-742 of SEQ ID NO: 4.

In some embodiments, the AAV9 capsid variant comprises: (i) a VP1 protein comprising an amino acid sequence having at least 95% identity to SEQ ID NO: 4; (ii) a VP2 protein comprising an amino acid sequence having at least 95% identity to positions 138-742 SEQ ID NO: 4; and/or (iii) a VP3 protein comprising an amino acid sequence having at least 95% identity to positions 203-742 of SEQ ID NO: 4.

In some embodiments, the AAV9 capsid variant comprises: (i) a VP1 protein comprising an amino acid sequence having at least 99% identity to SEQ ID NO: 4; (ii) a VP2 protein comprising an amino acid sequence having at least 99% identity to positions 138-742 of SEQ ID NO: 4; and/or (iii) a VP3 protein comprising an amino acid sequence having at least 99% identity to positions 203-742 of SEQ ID NO: 4.

In some embodiments, the AAV9 capsid variant comprises: (i) a VP1 protein comprising the amino acid sequence of SEQ ID NO: 4; (ii) a VP2 protein comprising the amino acid sequence of positions 138-742 of SEQ ID NO: 4; and/or (iii) a VP3 protein comprising the amino acid sequence of positions 203-742 of SEQ ID NO: 4.

In some embodiments, the AAV9 capsid variant comprises: (i) the amino acid sequence of SPHSKA (SEQ ID NO: 941), wherein the amino acid sequence is present immediately subsequent to an amino acid position corresponding to position 455 of SEQ ID NO: 4; (ii) an E at an amino acid position corresponding to position 451 and a V at an amino acid position corresponding to position 453 of SEQ ID NO: 4; and (iii) no other modifications relative to wild type AAV9.

In some embodiments, the AAV9 capsid variant further comprises one, two, or all of: an E at an amino acid position corresponding to position 451, an R at an amino acid position corresponding to position 452, and/or a V at an amino acid position corresponding to position 453 of SEQ ID NO: 36. In some embodiments, the AAV9 capsid variant comprises the amino acid sequence of KTERVSGSPHSKAQNQQT (SEQ ID NO: 3589).

In some embodiments, the AAV9 capsid variant comprises: (i) a VP1 protein comprising an amino acid sequence having at least 90% identity to SEQ ID NO: 36; (ii) a VP2 protein comprising an amino acid sequence having at least 90% identity to positions 138-742 SEQ ID NO: 36; and/or (iii) a VP3 protein comprising an amino acid sequence having at least 90% identity to positions 203-742 of SEQ ID NO: 36.

In some embodiments, the AAV9 capsid variant comprises: (i) a VP1 protein comprising an amino acid sequence having at least 95% identity to SEQ ID NO: 36; (ii) a VP2 protein comprising an amino acid sequence having at least 95% identity to positions 138-742 SEQ ID NO: 36; and/or (iii) a VP3 protein comprising an amino acid sequence having at least 95% identity to positions 203-742 of SEQ ID NO: 36.

In some embodiments, the AAV9 capsid variant comprises: (i) a VP1 protein comprising an amino acid sequence having at least 99% identity to SEQ ID NO: 36; (ii) a VP2 protein comprising an amino acid sequence having at least 99% identity to positions 138-742 of SEQ ID NO: 36; and/or (iii) a VP3 protein comprising an amino acid sequence having at least 99% identity to positions 203-742 of SEQ ID NO: 36.

In some embodiments, the AAV9 capsid variant comprises: (i) a VP1 protein comprising the amino acid sequence of SEQ ID NO: 36; (ii) a VP2 protein comprising the amino acid sequence of positions 138-742 of SEQ ID NO: 36; and/or (iii) a VP3 protein comprising the amino acid sequence of positions 203-742 of SEQ ID NO: 36.

In some embodiments, the AAV9 capsid variant comprises: (i) the amino acid sequence SPHSKA (SEQ ID NO: 941), wherein the amino acid sequence is present immediately subsequent to an amino acid position corresponding to position 455 of SEQ ID NO: 36; (ii) an E at an amino acid position corresponding to position 451, an R at an amino acid position corresponding to position 452, and a V at an amino acid position corresponding to position 453 of SEQ ID NO: 36; and (iii) no other modifications relative to wild type AAV9.

In some embodiments, the present disclosure provides an AAV particle comprising an AAV capsid variant comprising an amino acid sequence having the following formula: [N1]-[N2]-[N3], wherein the [N1]-[N2]-[N3] is present immediately subsequent to a position corresponding to the amino acid position 452 of SEQ ID NO: 982; and wherein the AAV capsid variant comprises an amino acid sequence at least 90% identical, e.g., at least 91%, at least 92%, at least 93%, at least 94%, at least 95%, at least 96%, at least 97%, at least 98%, at least 99%, or 100% identical, to the amino acid sequence of SEQ ID NO: 982, e.g., to positions 203-742 of SEQ ID NO: 982.

In some embodiments. [N1] comprises GHD. In some embodiments, [N1] comprises the amino acid G at a position corresponding to position 453, the amino acid H at position 454, and the amino acid D at position 455 of SEQ ID NO: 138 or SEQ ID NO: 982. In some embodiments. [N3] comprises KSG.

In some embodiments, the AAV capsid variant comprises: (i) a VP1 protein comprising the amino acid sequence of SEQ ID NO: 982 or an amino acid sequence having at least 90% identity to SEQ ID NO: 982; (ii) a VP2 protein comprising the amino acid sequence of positions 138-742 of SEQ ID NO: 982 or an amino acid sequence having at least 90% identity to positions 138-742 SEQ ID NO: 982; or (iii) a VP3 protein comprising the amino acid sequence of positions 203-742 of SEQ ID NO: 982 or an amino acid sequence having at least 90% identity to positions 203-742 of SEQ ID NO: 982.

In some embodiments, the AAV capsid variant comprises: (i) a VP1 protein comprising the amino acid sequence of SEQ ID NO: 982 or an amino acid sequence having at least 95% identity to SEQ ID NO: 982; (ii) a VP2 protein comprising the amino acid sequence of positions 138-742 of SEQ ID NO: 982 or an amino acid sequence having at least 95% identity to positions 138-742 SEQ ID NO: 982; or (iii) a VP3 protein comprising the amino acid sequence of positions 203-742 of SEQ ID NO: 982 or an amino acid sequence having at least 95% identity to positions 203-742 of SEQ ID NO: 982.

In some embodiments, the AAV capsid variant comprises: (i) a VP1 protein comprising the amino acid sequence of SEQ ID NO: 982 or an amino acid sequence having at least 99% identity to SEQ ID NO: 982; (ii) a VP2 protein comprising the amino acid sequence of positions 138-742 of SEQ ID NO: 982 or an amino acid sequence having at least 99% identity to positions 138-742 SEQ ID NO: 982; or (iii) a VP3 protein comprising the amino acid sequence of positions 203-742 of SEQ ID NO: 982 or an amino acid sequence having at least 99% identity to positions 203-742 of SEQ ID NO: 982.

In some embodiments, the AAV capsid variant comprises: (i) a VP1 protein comprising the amino acid sequence of SEQ ID NO: 982; (ii) a VP2 protein comprising the amino acid sequence of positions 138-742 of SEQ ID NO: 982; or (iii) a VP3 protein comprising the amino acid sequence of positions 203-742 of SEQ ID NO: 982.

In some embodiments, the present disclosure provides an AAV particle comprising a viral genome encoding a wildtype STXBP1 protein. In some embodiments, the viral genome encodes a human STXBP1 protein. In some embodiments, the STXBP1 protein comprises the amino acid sequence of SEQ ID NO: 6413.

In some embodiments, the STXBP1-encoding sequence comprises a nucleotide sequence that is at least 90% identical (e.g., at least 90% at least 91%, at least 92%, at least 93%, at least 94%, at least 95%, at least 96%, at least 97%, at least 98%, at least 99%, or 100% identical) to SEQ ID NO: 6414. In some embodiments, the STXBP1-encoding sequence comprises a nucleotide sequence that is at least 95% identical to SEQ ID NO: 6414. In some embodiments, the STXBP1-encoding sequence comprises a nucleotide sequence that is at least 99% identical to SEQ ID NO: 6414. In some embodiments, the STXBP1-encoding sequence comprises the nucleotide sequence of SEQ ID NO: 6414. In some embodiments, the STXBP1-encoding sequence consists of the nucleotide sequence of SEQ ID NO: 6414.

In some embodiments, the viral genome comprises a promoter operably linked to the STXBP1-encoding sequence. In some embodiments, the promoter is human elongation factor 1α-subunit (EF1α), cytomegalovirus (CMV) immediate-early enhancer and/or promoter, chicken β-actin (CBA), CAG, CAG derivative, β glucuronidase (GUSB), or ubiquitin C (UBC), neuron-specific enolase (NSE), platelet-derived growth factor (PDGF), platelet-derived growth factor B-chain (PDGF-0), intercellular adhesion molecule 2 (ICAM-2), synapsin (Syn), synapsin 1 (Syn1), methyl-CpG binding protein 2 (MeCP2), Ca2+/calmodulin-dependent protein kinase II (CaMKII), metabotropic glutamate receptor 2 (mGluR2), neurofilamcnt light (NFL) or heavy (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof.

In some embodiments, the viral genome further comprises an inverted terminal repeat (ITR) sequence. In some embodiments, the viral genome comprises an ITR sequence positioned 5′ relative to the STXBP1-encoding sequence. In some embodiments, the viral genome comprises an ITR sequence positioned 3′ relative to the STXBP1-encoding sequence. In some embodiments, the viral genome comprises an ITR sequence positioned 5′ relative to the STXBP1-encoding sequence and an ITR sequence positioned 3′ relative to the STXBP1-encoding sequence.

In some embodiments, the present disclosure provides an AAV particle comprising a viral genome comprising a syntaxin-binding protein 1 (STXBP1)-encoding sequence and an AAV capsid variant comprising: (i) a VP1 protein comprising the amino acid sequence of SEQ ID NO: 4; (ii) a VP2 protein comprising the amino acid sequence of positions 138-742 of SEQ ID NO: 4; and/or (iii) a VP3 protein comprising the amino acid sequence of positions 203-742 of SEQ ID NO: 4.

In some embodiments, the present disclosure provides an AAV particle comprising a viral genome comprising syntaxin-binding protein 1 (STXBP1)-encoding sequence and an AAV capsid variant comprising: (i) a VP1 protein comprising the amino acid sequence of SEQ ID NO: 36; (ii) a VP2 protein comprising the amino acid sequence of positions 138-742 of SEQ ID NO: 36; and/or (iii) a VP3 protein comprising the amino acid sequence of positions 203-742 of SEQ ID NO: 36.

In some embodiments, the present disclosure provides a cell comprising an AAV particle disclosed herein. In some embodiments, the cell is a mammalian cell (e.g., an HEK293 cell), an insect cell (e.g., an Sf9 cell), or a bacterial cell.

In some embodiments, the present disclosure provides a method of making an AAV particle disclosed herein, the method comprising: (i) providing a cell comprising the viral genome comprising a STXBP1-encoding sequence and a nucleic acid encoding the AAV capsid variant; and (ii) incubating the cell under conditions suitable to encapsulate the viral genome in the AAV capsid variant; thereby making the AAV particle.

In some embodiments, the viral genome of the AAV particle comprises the nucleotide sequence of SEQ ID NO: 6414 or a nucleotide sequence that is at least 90% identical (e.g., at least 90%, at least 91%, at least 92%, at least 93%, at least 94%, at least 95%, at least 96%, at least 97%, at least 98%, or at least 99% identical) thereto; and the AAV capsid variant of the AAV particle comprises: (i) a VP1 protein comprising the amino acid sequence of SEQ ID NO: 4 or an amino acid sequence having at least 90% identity (e.g., at least 90%, at least 91%, at least 92%, at least 93%, at least 94%, at least 95%, at least 96%, at least 97%, at least 98%, or at least 99% identity) to SEQ ID NO: 4; (ii) a VP2 protein comprising the amino acid sequence of positions 138-742 of SEQ ID NO: 4 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 138-742 SEQ ID NO: 4; or (iii) a VP3 protein comprising the amino acid sequence of positions 203-742 of SEQ ID NO: 4 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 203-742 of SEQ ID NO: 4.

In some embodiments, the AAV capsid variant comprises the amino acid sequence of SEQ ID NO: 4, the amino acid sequence of positions 138-742 of SEQ ID NO: 4, and/or the amino acid sequence of positions 203-742 of SEQ ID NO: 4.

In some embodiments, the viral genome of the AAV particle comprises the nucleotide sequence of SEQ ID NO: 6414 or a nucleotide sequence that is at least 90% identical (e.g., at least 90%0, at least 91%, at least 92%, at least 93%, at least 94%, at least 95%, at least 6%, at least 97%, at least 98%, or at least 99% identical) thereto; and the AAV capsid variant of the AAV particle comprises (i) a VP1 protein comprising the amino acid sequence of SEQ ID NO: 36 or an amino acid sequence having at least 90% identity (e.g., at least 90%, at least 91%, at least 92%, at least 93%, at least 94%, at least 95%, at least 96%, at least 97%, at least 98%, or at least 99% identity) to SEQ ID NO: 36; (ii) a VP2 protein comprising the amino acid sequence of positions 138-742 of SEQ ID NO: 36 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 138-742 SEQ ID NO: 36; or (iii) a VP3 protein comprising the amino acid sequence of positions 203-742 of SEQ ID NO: 36 or an amino acid sequence having at least 90% identity (e.g., at least 90%, at least 91%, at least 92%, at least 93%, at least 94%, at least 95%, at least 696%, at least 97%, at least 98%, or at least 99% identity) to positions 203-742 of SEQ ID NO: 36.

In some embodiments, the AAV capsid variant comprises the amino acid sequence of SEQ ID NO: 36, the amino acid sequence of positions 138-742 of SEQ ID NO: 36, and/or the amino acid sequence of positions 203-742 of SEQ ID NO: 36.

In some embodiments, the viral genome of the AAV particle comprises the nucleotide sequence of SEQ ID NO: 6414 or a nucleotide sequence that is at least 90% identical (e.g., at least 90%, at least 91%, at least 92%, at least 93%, at least 94%, at least 95%, at least 96%, at least 97%, at least 98%, or at least 99% identical) thereto; and the AAV capsid variant of the AAV particle comprises: (i) a VP1 protein comprising the amino acid sequence of SEQ ID NO: 982 or an amino acid sequence having at least 90% identity (e.g., at least 90%, at least 91%, at least 92%, at least 93%, at least 94%, at least 95%, at least 96%, at least 97%, at least 98%, or at least 99% identity) to SEQ ID NO: 982; (ii) a VP2 protein comprising the amino acid sequence of positions 138-742 of SEQ ID NO: 982 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 138-742 SEQ ID NO: 982; or (iii) a VP3 protein comprising the amino acid sequence of positions 203-742 of SEQ ID NO: 982 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 203-742 of SEQ ID NO: 982.

In some embodiments, the AAV capsid variant comprises the amino acid sequence of SEQ ID NO: 982, the amino acid sequence of positions 138-742 of SEQ ID NO: 982, and/or the amino acid sequence of positions 203-742 of SEQ ID NO: 982.

In some embodiments, the method of making further comprises, prior to step (i), introducing a first nucleic acid molecule comprising the viral genome into the cell.

In some embodiments, the cell comprises a second nucleic acid molecule encoding the AAV capsid variant. In some embodiments, the method of making further comprises, prior to step (i), introducing the second nucleic acid molecule into the cell.

In some embodiments, the cell comprises a mammalian cell (e.g., an HEK293 cell), an insect cell (e.g., an Sf9 cell), or a bacterial cell.

In some embodiments, the present disclosure provides a pharmaceutical composition comprising an AAV particle disclosed herein and a pharmaceutically acceptable excipient.

In some embodiments, the present disclosure provides a method of delivering an AAV particle encoding an STXBP1 protein to a subject, comprising administering to the subject an effective amount of a pharmaceutical composition or AAV particle disclosed herein.

In some embodiments, the present disclosure provides a method of treating a subject having or diagnosed with having an STXBP1-related disorder, comprising administering to the subject an effective amount of a pharmaceutical composition or AAV particle disclosed herein. In some embodiments, the STXBP1-related disorder is a STXBP1-related neurodegenerative or neuromuscular disorder. In some embodiments, the STXBP1-related neurodegenerative or neuromuscular disorder is STXBP1 encephalopathy, epileptic encephalopathy, Ohtahara syndrome, developmental encephalopathy. West syndrome, early myoclonic epileptic encephalopathy, Lennox-Gaustaut syndrome, autism (e.g., autism with STXBP1 mutations and optionally further mutations). Dravet syndrome (not caused by mutations in SCN1A), or Rett syndrome phenotype (not caused by mutation of MECP2 or CDKL5).

In some embodiments, the present disclosure provides a method of treating a subject having STXBP1 encephalopathy or diagnosed with having STXBP1 encephalopathy, comprising administering to the subject an effective amount of a pharmaceutical composition or AAV particle disclosed herein.

In some embodiments, the subject has one or more mutations in the STXBP1 gene.

In some embodiments, the subject has a reduced level of STXBP1 activity as compared to a reference level in a subject who does not have an STXBP1-related disorder.

In some embodiments, the treating results in prevention of progression of the disorder in the subject. In some embodiments, the treating results in amelioration of the disorder. In some embodiments, the treating results in a change in one or more biomarkers of the disorder. In some embodiments, the one or more biomarkers comprises an STXBP1 activity or neurofilament light chain.

In some embodiments, the treating results in amelioration of at least one symptom of the disorder. In some embodiments, the at least one symptom comprises epilepsy, autistic features, ataxia, generalized tremors, dystonia, or a combination thereof.

In some embodiments, the subject is a human.

In some embodiments, the AAV particle is delivered to a cell, tissue, or region of the CNS, e.g., a region of the brain or spinal cord, e.g., the parenchyma, the cortex, substantia nigra, caudate cerebellum, striatum, corpus callosum, cerebellum, brain stem caudate-putamen, thalamus, superior colliculus, the spinal cord, or a combination thereof, and/or to neurons, e.g. GABAergic neurons, glutamatergic neurons, or a combination thereof.

In any one of the preceding embodiments, the method of delivering or treating further comprises evaluating, e.g., measuring, the level of STXBP1 expression, e.g., STXBP1 gene expression, STXBP1 mRNA expression, and/or STXBP1 protein expression, in the subject, e.g., in a cell, tissue, or fluid of the subject. In some embodiments, the level of STXBP1 protein expression is measured by an ELISA, a Western blot, or an immunohistochemistry assay.

In some embodiments, evaluating the level of STXBP1 expression is performed prior to and subsequent to administration of the AAV particle, optionally wherein the level of STXBP1 expression prior to treatment is compared to the level of STXBP1 expression subsequent to administration. In some embodiments, the level of STXBP1 expression is evaluated in a cell or tissue of the central nervous system (e.g., parenchyma) from the subject. In some embodiments, the subject's level of STXBP1 protein expression subsequent to administration is increased relative to the subject's level of STXBP1 protein expression prior to administration.

In some embodiments, the administration of a pharmaceutical composition or AAV particle described herein results in an increase in: (i) STXBP1 activity in a cell, tissue, (e.g., a cell or tissue of the CNS, e.g., the cortex, striatum, thalamus, cerebellum, and/or brainstem), and/or fluid (e.g., CSF and/or serum) of the subject, relative to STXBP1 activity in the subject prior to the administration; (ii) viral genomes (VG) per cell level in a CNS tissue (e.g., the cortex, striatum, thalamus, cerebellum, brainstem, and/or spinal cord) of the subject, relative to the subject's VG per cell level in a peripheral tissue, and/or (iii) STXBP1 mRNA expression in a cell or tissue (e.g., a cell or tissue of the CNS, e.g., the cortex, thalamus, and/or brainstem) of the subject, as compared to STXBP1 mRNA expression in the subject prior to the administration.

In some embodiments, the method of treating further comprises administering to the subject an additional agent suitable for treatment or prevention of an STXBP1-related disorder. In some embodiments, the additional agent comprises one or more anti-epileptic drugs (e.g., levetiracetam, phenobarbital, clobazam, topiramate), adrenocorticotropic hormone, or a combination thereof. In some embodiments, the method further comprises administering an immunosuppressant to the subject. In some embodiments, the immunosuppressant comprises a corticosteroid (e.g., prednisone, prednisolone, methylprednisolone, and/or dexamethasone), rapanycin, mycophenolate mofetil, tacrolimus, rituximab, and/or eculizumab hydroxychloroquine.

In some embodiments, the present disclosure provides a pharmaceutical composition or AAV particle described herein for use in the treatment of an STXBP1-related disorder. In some embodiments, the STXBP1-related disorder is STXBP1 encephalopathy, epileptic encephalopathy. Ohtahara syndrome, developmental encephalopathy, West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5).

In some embodiments, the present disclosure provides a use of a pharmaceutical composition or AAV particle described herein in the manufacture of a medicament for the treatment of an STXBP1-related disorder. In some embodiments, the STXBP1-related disorder is STXBP1 encephalopathy, epileptic encephalopathy, Ohtahara syndrome, developmental encephalopathy. West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5).

### Enumerated Embodiments

1. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises an amino acid sequence having the following formula: [N1]-[N2]-[N3], wherein:
(i) optionally [N1] comprises X1, X2, and X3, wherein at least one of X1, X2, or X3 is G;
(ii) [N2] comprises the amino acid sequence of SPH; and
(iii) [N3] comprises X4, X5, and X6, wherein at least one of X4. X5, or X6 is a basic amino acid, e.g., a K or R.
   2. The AAV particle of embodiment 1, wherein X4, X5, or both of [N3] is a K.
   3. The AAV particle of embodiment 1 or 2, wherein X4, X5, or X6 of [N3] is an R.
   4. The AAV particle of any one of embodiments 1-3, wherein:
(a) X4 of [N3] is: K, S, A, V, T, G, F, W, V, N, or R;
(b) X5 of [N3] is: S, K, T, FL, L, Y, H, M, or R; and/or
(c) X6 of [N3] is: G, A, R, M, I, N, T, Y, D, P, V, L, E, W, N, Q, K, or S;
optionally wherein the AAV capsid variant comprises an amino acid modification, e.g., a conservative substitution, of any of the aforesaid amino acids in (a)-(c).
   5. The AAV particle of any one of embodiments 1-4, wherein [N3] comprises SK, KA, KS, AR, RM, VK, AS, SR, VK, KR, KK, KN, VR, RS, RK, KT, TS, KF, FG, KI, IG, KL, LG. TT. TY, KY, YG, KD, KP, TR, RG, VR, GA, SL, SS, FL, WK, SA, RA, LR, KW, RR, GK, TK, NK, AK, KV, KG, KH, KM, TG, SE, SV, SW, SN, HG, SQ, LW, MG, MA, or SG.
   6. The AAV particle of any one of embodiments 1-5, wherein [N3] is or comprises SKA, KSG, ARM, VKS, ASR, VKI, KKN, VRM, RKA, KTS, KFG, KIG, KLG, KTT, KTY, KYG, SKD, SKP, TRG, VRG, KRG, GAR, KSA, KSR, SKL, SRA, SKR, SLR, SRG, SSR, FLR, SKW, SKS, WKA, VRR, SKV, SKT, SKG, GKA, TKA, NKA, SKL, SKN, AKA, KTG, KSL, KSE, KSV, KSW, KSN, KHG, KSQ, KSK, KLW, WKG, KMG, KMA, or RSG.
   7. The AAV particle of any one of embodiments 1-4, wherein [N2]-[N3] comprises SPHSK (SEQ ID NO: 4701), SPHKS (SEQ ID NO: 4704), SPHAR (SEQ ID NO: 4705), SPHVK (SEQ ID NO: 4706), SPHAS (SEQ ID NO: 4707), SPHKK (SEQ ID NO: 4708), SPHVR (SEQ ID NO: 4709), SPHRK (SEQ ID NO: 4710), SPHKT (SEQ ID NO: 4711), SPHKF (SEQ ID NO: 4712), SPHKI (SEQ ID NO: 4713), SPHKL (SEQ ID NO: 4714), SPHKY (SEQ ID NO: 4715), SPHTR (SEQ ID NO: 4716), SPHKR (SEQ ID NO: 4717), SPHGA (SEQ ID NO: 4718), SPHSR (SEQ ID NO: 4719), SPHSL (SEQ ID NO: 4720), SPHSS (SEQ ID NO: 4721), SPHFL (SEQ ID NO: 4722), SPHWK (SEQ ID NO: 4723), SPHGK (SEQ ID NO: 4724), SPHTK (SEQ ID NO: 4725), SPHNK (SEQ ID NO: 4726), SPHAK (SEQ ID NO: 4727), SPHKH (SEQ ID NO: 4728), SPHKM (SEQ ID NO: 4729), or SPHRS (SEQ ID NO: 4730). 8. The AAV particle of any one of embodiments 1-7, wherein [N2]-[N3] is or comprises:

(i)
(SEQ ID NO: 941)
SPHSKA,
(SEQ ID NO: 946)
SPHKSG,
(SEQ ID NO: 947)
SPHARM,
(SEQ ID NO: 948)
SPHVKS,
(SEQ ID NO: 949)
SPHASR,
(SEQ ID NO: 950)
SPHVKI,
(SEQ ID NO: 954)
SPHKKN,
(SEQ ID NO: 955)
SPHVRM,
(SEQ ID NO: 956)
SPHRKA,
(SEQ ID NO: 957)
SPHKFG,
(SEQ ID NO: 958)
SPHKIG,
(SEQ ID NO: 959)
SPHKLG,
(SEQ ID NO: 963)
SPHKTS,
(SEQ ID NO: 964)
SPHKTT,
(SEQ ID NO: 965)
SPHKTY,
(SEQ ID NO: 966)
SPHKYG,
(SEQ ID NO: 967)
SPHSKD,
(SEQ ID NO: 968)
SPHSKP,
(SEQ ID NO: 972)
SPHTRG,
(SEQ ID NO: 973)
SPHVRG,
(SEQ ID NO: 974)
SPHKRG,
(SEQ ID NO: 975)
SPHGAR,
(SEQ ID NO: 977)
SPHKSA,
(SEQ ID NO: 951)
SPHKSR,
(SEQ ID NO: 960)
SPHSKL,
(SEQ ID NO: 969)
SPHSRA,
(SEQ ID NO: 978)
SPHSKR,
(SEQ ID NO: 952)
SPHSLR,
(SEQ ID NO: 961)
SPHSRG,
(SEQ ID NO: 970)
SPHSSR,
(SEQ ID NO: 979)
SPHFLR,
(SEQ ID NO: 953)
SPHSKW,
(SEQ ID NO: 962)
SPHSKS,
(SEQ ID NO: 971)
SPHWKA,
(SEQ ID NO: 980)
SPHVRR,
(SEQ ID NO: 4731)
SPHSKT,
(SEQ ID NO: 4732)
SPHSKG,
(SEQ ID NO: 4733)
SPHGKA,
(SEQ ID NO: 4734)
SPHNKA,
(SEQ ID NO: 4735)
SPHSKN,
(SEQ ID NO: 4736)
SPHAKA,
(SEQ ID NO: 4737)
SPHSKV,
(SEQ ID NO: 4738)
SPHKTG,
(SEQ ID NO: 4739)
SPHTKA,
(SEQ ID NO: 4740)
SPHKSL,
(SEQ ID NO: 4741)
SPHKSE,
(SEQ ID NO: 4742)
SPHKSV,
(SEQ ID NO: 4743)
SPHKSW,
(SEQ ID NO: 4744)
SPHKSN,
(SEQ ID NO: 4745)
SPHKHG,
(SEQ ID NO: 4746)
SPHKSQ,
(SEQ ID NO: 4747)
SPHKSK,
(SEQ ID NO: 4748)
SPHKLW,
(SEQ ID NO: 4749)
SPHWKG,
(SEQ ID NO: 4750)
SPHKMG,
(SEQ ID NO: 4751)
SPHKMA,
or
(SEQ ID NO: 976)
SPHRSG;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   9. The AAV particle of any one of embodiments 1-8, wherein the AAV capsid variant comprises an amino acid other than G at position 453 (e.g., V, R, D, E, M, T, I, S, A, N, L, K, H, P, W, or C), an amino acid other than S at position 454 (e.g., V, L, N, D, H, R, P, G, amino acid other than G at position 455 (e.g., C, L, D, E, Y, H, V, A, N, P, or S), numbered according to any one of SEQ ID NOs: 36-59, 138, 981, or 982.
   10. The AAV particle of any one of embodiments 1-8, wherein the AAV capsid variant comprises the amino acid G at position 453, the amino acid S at position 454, and the amino acid G at position 455, numbered according to SEQ ID NO: 138 or 981.
   11. The AAV particle of any one of embodiments 1-9, wherein the AAV capsid variant comprises the amino acid G at position 453, the amino acid H at position 454, and the amino acid D at position 455, numbered according to SEQ ID NO: 138 or 982.
   12. The AAV particle of any one of embodiments 1-11, wherein [N1] comprises X1, X2, and X3, wherein at least one of X1, X2, or X3 is G.
   13. The AAV particle of any one of embodiments 1-12, wherein:
(a) X1 of [N1] is: G, V, R, D, E, M, T, I, S, A, N, L, K, H, P, W, or C;
(b) X2 of [N1] is: S, V, L, N, D, H, R, P, G, T, I, A, E, Y, M, or Q; and/or
(c) X3 of [N1] is: G, C, L, D, E, Y, H, V, A, N, P, or S;
optionally wherein the AAV capsid variant comprises an amino acid modification, e.g., a conservative substitution, of any of the aforesaid amino acids in (a)-(c).
   14. The AAV particle of any one of embodiments 1-13, wherein [N1] comprises GS. SG. GH, HD, GQ, QD, VS, CS, GR, RG, QS, SH, MS, RN, TS, IS, GP, ES, SS, GN, AS, NS, LS, GG, KS, GT, PS, RS, GI, WS, DS, ID, GL, DA, DG, ME, EN, KN, KE, AI, NG, PG, TG, SV, IG, LG, AG, EG, SA, YD, HE, HG, RD, ND, PD, MG, QV, DD, HN, HP, GY, GM, GD, or HS.
   15. The AAV particle of any one of embodiments 1-14, wherein [N1] is or comprises GSG, GHD, GQD, VSG, CSG, GRG, CSH, GQS, GSH, RVG, GSC, GLL, GDD, GHE, GNY, MSG, RNG, TSG, iSG, GPG, ESG, SSG, GNG, ASG, NSG, LSG, GGG, KSG, HSG, GTG, PSG, GSV, RSG, GIG, WSG, DSG, IDG, GLG, DAG, DGG, MEG, ENG, GSA, KNG, KEG, AIG, GYD, GHG, GRD, GND, GPD, GMG, GQV, GHN, GHP, or GHS.

16. The AAV particle of any one of embodiments 1-15, wherein [N1]-[N2] comprises:

(i)
(SEQ ID NO: 4752)
SGSPH,
(SEQ ID NO: 4703)
HDSPH,
(SEQ ID NO: 4753)
QDSPH,
(SEQ ID NO: 4754)
RGSPH,
(SEQ ID NO: 4755)
SHSPH,
(SEQ ID NO: 4756)
QSSPH,
(SEQ ID NO: 4757)
DDSPH,
(SEQ ID NO: 4758)
HESPH,
(SEQ ID NO: 4759)
NYSPH,
(SEQ ID NO: 4760)
VGSPH,
(SEQ ID NO: 4761)
SCSPH,
(SEQ ID NO: 4762)
LLSPH,
(SEQ ID NO: 4763)
NGSPH,
(SEQ ID NO: 4764)
PGSPH,
(SEQ ID NO: 4765)
GGSPH,
(SEQ ID NO: 4766)
TGSPH,
(SEQ ID NO: 4767)
SVSPH,
(SEQ ID NO: 4768)
IGSPH,
(SEQ ID NO: 4769)
DGSPH,
(SEQ ID NO: 4770)
LGSPH,
(SEQ ID NO: 4771)
AGSPH,
(SEQ ID NO: 4772)
EGSPH,
(SEQ ID NO: 4773)
SASPH,
(SEQ ID NO: 4774)
YDSPH,
(SEQ ID NO: 4775)
HGSPH,
(SEQ ID NO: 4776)
RDSPH,
(SEQ ID NO: 4777)
NDSPH,
(SEQ ID NO: 4778)
PDSPH,
(SEQ ID NO: 4779)
MGSPH,
(SEQ ID NO: 4780)
QVSPH,
(SEQ ID NO: 4781)
HNSPH,
(SEQ ID NO: 4782)
HPSPH,
or
(SEQ ID NO: 4783)
HSSPH;

(ii) an amino acid sequence comprising any portion of an amino acid sequence in (i), e.g., any 2, 3, or 4 amino acids, e.g., consecutive amino acids, thereof.
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   17. The AAV particle of any one of embodiments 1-16, wherein [N1]-[N2] is or comprises:

(i)
(SEQ ID NO: 4695)
GSGSPH,
(SEQ ID NO: 4784)
GHDSPH,
(SEQ ID NO: 4785)
GQDSPH,
(SEQ ID NO: 4786)
VSGSPH,
(SEQ ID NO: 4787)
CSGSPH,
(SEQ ID NO: 4788)
GRGSPH,
(SEQ ID NO: 4789)
CSHSPH,
(SEQ ID NO: 4790)
GQSSPH,
(SEQ ID NO: 4791)
GSHSPH,
(SEQ ID NO: 4792)
GDDSPH,
(SEQ ID NO: 4793)
GHESPH,
(SEQ ID NO: 4794)
GNYSPH,
(SEQ ID NO: 4795)
RVGSPH,
(SEQ ID NO: 4796)
GSCSPH,
(SEQ ID NO: 4797)
GLLSPH,
(SEQ ID NO: 4798)
MSGSPH,
(SEQ ID NO: 4799)
RNGSPH,
(SEQ ID NO: 4800)
TSGSPH,
(SEQ ID NO: 4801)
ISGSPH,
(SEQ ID NO: 4802)
GPGSPH,
(SEQ ID NO: 4803)
ESGSPH,
(SEQ ID NO: 4804)
SSGSPH,
(SEQ ID NO: 4805)
GNGSPH,
(SEQ ID NO: 4806)
ASGSPH,
(SEQ ID NO: 4807)
NSGSPH,
(SEQ ID NO: 4808)
LSGSPH,
(SEQ ID NO: 4809)
GGGSPH,
(SEQ ID NO: 4810)
KSGSPH,
(SEQ ID NO: 4811)
HSGSPH,
(SEQ ID NO: 4812)
GTGSPH,
(SEQ ID NO: 4813)
PSGSPH,
(SEQ ID NO: 4814)
GSVSPH,
(SEQ ID NO: 4815)
RSGSPH,
(SEQ ID NO: 4816)
GIGSPH,
(SEQ ID NO: 4817)
WSGSPH,
(SEQ ID NO: 4818)
DSGSPH,
(SEQ ID NO: 4819)
IDGSPH,
(SEQ ID NO: 4820)
GLGSPH,
(SEQ ID NO: 4821)
DAGSPH,
(SEQ ID NO: 4822)
DGGSPH,
(SEQ ID NO: 4823)
MEGSPH,
(SEQ ID NO: 4824)
ENGSPH,
(SEQ ID NO: 4825)
GSASPH,
(SEQ ID NO: 4826)
KNGSPH,
(SEQ ID NO: 4827)
KEGSPH,
(SEQ ID NO: 4828)
AIGSPH,
(SEQ ID NO: 4829)
GYDSPH,
(SEQ ID NO: 4830)
GHGSPH,
(SEQ ID NO: 4831)
GRDSPH,
(SEQ ID NO: 4832)
GNDSPH,
(SEQ ID NO: 4833)
GPDSPH,
(SEQ ID NO: 4834)
GMGSPH,
(SEQ ID NO: 4835)
GQVSPH,
(SEQ ID NO: 4836)
GHNSPH,
(SEQ ID NO: 4837)
GHPSPH,
or
(SEQ ID NO: 4838)
GHSSPH;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   18. The AAV particle of any one of embodiments 1-17, wherein [N1]-[N2]-[N3] comprises:

(i)
(SEQ ID NO: 4839)
SGSPHSK,
(SEQ ID NO: 4840)
HDSPHKS,
(SEQ ID NO: 4841)
SGSPHAR,
(SEQ ID NO: 4842)
SGSPHVK,
(SEQ ID NO: 4843)
QDSPHKS,
(SEQ ID NO: 4844)
SGSPHKK,
(SEQ ID NO: 4845)
SGSPHVR,
(SEQ ID NO: 4846)
SGSPHAS,
(SEQ ID NO: 4847)
SGSPHRK,
(SEQ ID NO: 4848)
SGSPHKT,
(SEQ ID NO: 4849)
SHSPHKS,
(SEQ ID NO: 4850)
QSSPHRS,
(SEQ ID NO: 4851)
RGSPHAS,
(SEQ ID NO: 4852)
RGSPHSK,
(SEQ ID NO: 4853)
SGSPHKF,
(SEQ ID NO: 4854)
SGSPHKI,
(SEQ ID NO: 4855)
SGSPHKL,
(SEQ ID NO: 4856)
SGSPHKY,
(SEQ ID NO: 4857)
SGSPHTR,
(SEQ ID NO: 4858)
SHSPHKR,
(SEQ ID NO: 4859)
SGSPHGA,
(SEQ ID NO: 4860)
HDSPHKR,
(SEQ ID NO: 4861)
DDSPHKS,
(SEQ ID NO: 4862)
HESPHKS,
(SEQ ID NO: 4863)
NYSPHKI,
(SEQ ID NO: 4864)
SGSPHSR,
(SEQ ID NO: 4865)
SGSPHSL,
(SEQ ID NO: 4866)
SGSPHSS,
(SEQ ID NO: 4867)
VGSPHSK,
(SEQ ID NO: 4868)
SCSPHRK,
(SEQ ID NO: 4869)
SGSPHFL,
(SEQ ID NO: 4870)
LLSPHWK,
(SEQ ID NO: 4871)
NGSPHSK,
(SEQ ID NO: 4872)
PGSPHSK,
(SEQ ID NO: 4873)
GGSPHSK,
(SEQ ID NO: 4874)
TGSPHSK,
(SEQ ID NO: 4875)
SVSPHGK,
(SEQ ID NO: 4876)
SGSPHTK,
(SEQ ID NO: 4877)
IGSPHSK,
(SEQ ID NO: 4878)
DGSPHSK,
(SEQ ID NO: 4879)
SGSPHNK,
(SEQ ID NO: 4880)
LGSPHSK,
(SEQ ID NO: 4881)
AGSPHSK,
(SEQ ID NO: 4882)
EGSPHSK,
(SEQ ID NO: 4883)
SASPHSK,
(SEQ ID NO: 4884)
SGSPHAK,
(SEQ ID NO: 4885)
HDSPHKI,
(SEQ ID NO: 4886)
YDSPHKS,
(SEQ ID NO: 4887)
HDSPHKT,
(SEQ ID NO: 4888)
RGSPHKR,
(SEQ ID NO: 4889)
HGSPHSK,
(SEQ ID NO: 4890)
RDSPHKS,
(SEQ ID NO: 4891)
NDSPHKS,
(SEQ ID NO: 4892)
QDSPHKI,
(SEQ ID NO: 4893)
PDSPHKI,
(SEQ ID NO: 4894)
PDSPHKS,
(SEQ ID NO: 4895)
MGSPHSK,
(SEQ ID NO: 4896)
HDSPHKH,
(SEQ ID NO: 4897)
QVSPHKS,
(SEQ ID NO: 4898)
HNSPHKS,
(SEQ ID NO: 4899)
NGSPHKR,
(SEQ ID NO: 4900)
HDSPHKY,
(SEQ ID NO: 4901)
NDSPHKI,
(SEQ ID NO: 4902)
HDSPHKL,
(SEQ ID NO: 4903)
HPSPHWK,
(SEQ ID NO: 4904)
HDSPHKM,
or
(SEQ ID NO: 4905)
HSSPHRS;

(ii) an amino acid sequence comprising any portion of an amino acid sequence in (i), e.g., any 2, 3, 4, 5, or 6 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   19. The AAV particle of any one of embodiments 1-18, wherein [N1]-[N2]-[N3] is or comprises:

(i)
(SEQ ID NO: 4697)
GSGSPHSKA,
(SEQ ID NO: 4698)
GHDSPHKSG,
(SEQ ID NO: 4906)
GSGSPHARM,
(SEQ ID NO: 4907)
GSGSPHVKS,
(SEQ ID NO: 4908)
GQDSPHKSG,
(SEQ ID NO: 4909)
GSGSPHASR,
(SEQ ID NO: 4910)
GSGSPHVKI,
(SEQ ID NO: 4911)
GSGSPHKKN,
(SEQ ID NO: 4912)
GSGSPHVRM,
(SEQ ID NO: 4913)
VSGSPHSKA,
(SEQ ID NO: 4914)
CSGSPHSKA,
(SEQ ID NO: 4915)
GSGSPHRKA,
(SEQ ID NO: 4916)
CSGSPHKTS,
(SEQ ID NO: 4917)
CSHSPHKSG,
(SEQ ID NO: 4918)
GQSSPHRSG,
(SEQ ID NO: 4919)
GRGSPHASR,
(SEQ ID NO: 4920)
GRGSPHSKA,
(SEQ ID NO: 4921)
GSGSPHKFG,
(SEQ ID NO: 4922)
GSGSPHKIG,
(SEQ ID NO: 4923)
GSGSPHKLG,
(SEQ ID NO: 4924)
GSGSPHKTS,
(SEQ ID NO: 4925)
GSGSPHKTT,
(SEQ ID NO: 4926)
GSGSPHKTY,
(SEQ ID NO: 4927)
GSGSPHKYG,
(SEQ ID NO: 4928)
GSGSPHSKD,
(SEQ ID NO: 4929)
GSGSPHSKP,
(SEQ ID NO: 4930)
GSGSPHTRG,
(SEQ ID NO: 4931)
GSGSPHVRG,
(SEQ ID NO: 4932)
GSHSPHKRG,
(SEQ ID NO: 4933)
GSHSPHKSG,
(SEQ ID NO: 4934)
VSGSPHASR,
(SEQ ID NO: 4935)
VSGSPHGAR,
(SEQ ID NO: 4936)
VSGSPHKFG,
(SEQ ID NO: 4937)
GHDSPHKRG,
(SEQ ID NO: 4938)
GDDSPHKSG,
(SEQ ID NO: 4939)
GHESPHKSA,
(SEQ ID NO: 4940)
GHDSPHKSA,
(SEQ ID NO: 4941)
GNYSPHKIG,
(SEQ ID NO: 4942)
GHDSPHKSR,
(SEQ ID NO: 4943)
GSGSPHSKL,
(SEQ ID NO: 4944)
GSGSPHSRA,
(SEQ ID NO: 4945)
GSGSPHSKR,
(SEQ ID NO: 4946)
GSGSPHSLR,
(SEQ ID NO: 4947)
GSGSPHSRG,
(SEQ ID NO: 4948)
GSGSPHSSR,
(SEQ ID NO: 4949)
RVGSPHSKA,
(SEQ ID NO: 4950)
GSCSPHRKA,
(SEQ ID NO: 4951)
GSGSPHFLR,
(SEQ ID NO: 4952)
GSGSPHSKW,
(SEQ ID NO: 4953)
GSGSPHSKS,
(SEQ ID NO: 4954)
GLLSPHWKA,
(SEQ ID NO: 4955)
GSGSPHVRR,
(SEQ ID NO: 4956)
GSGSPHSKV,
(SEQ ID NO: 4957)
MSGSPHSKA,
(SEQ ID NO: 4958)
RNGSPHSKA,
(SEQ ID NO: 4959)
TSGSPHSKA,
(SEQ ID NO: 4960)
ISGSPHSKA,
(SEQ ID NO: 4961)
GPGSPHSKA,
(SEQ ID NO: 4962)
GSGSPHSKT,
(SEQ ID NO: 4963)
ESGSPHSKA,
(SEQ ID NO: 4964)
SSGSPHSKA,
(SEQ ID NO: 4965)
GNGSPHSKA,
(SEQ ID NO: 4966)
ASGSPHSKA,
(SEQ ID NO: 4967)
NSGSPHSKA,
(SEQ ID NO: 4968)
LSGSPHSKA,
(SEQ ID NO: 4969)
GGGSPHSKA,
(SEQ ID NO: 4970)
KSGSPHSKA,
(SEQ ID NO: 4971)
GGGSPHSKS,
(SEQ ID NO: 4972)
GSGSPHSKG,
(SEQ ID NO: 4973)
HSGSPHSKA,
(SEQ ID NO: 4974)
GTGSPHSKA,
(SEQ ID NO: 4975)
PSGSPHSKA,
(SEQ ID NO: 4976)
GSVSPHGKA,
(SEQ ID NO: 4977)
RSGSPHSKA,
(SEQ ID NO: 4978)
GSGSPHTKA,
(SEQ ID NO: 4979)
GIGSPHSKA,
(SEQ ID NO: 4980)
WSGSPHSKA,
(SEQ ID NO: 4981)
DSGSPHSKA,
(SEQ ID NO: 4982)
IDGSPHSKA,
(SEQ ID NO: 4983)
GSGSPHNKA,
(SEQ ID NO: 4984)
GLGSPHSKS,
(SEQ ID NO: 4985)
DAGSPHSKA,
(SEQ ID NO: 4986)
DGGSPHSKA,
(SEQ ID NO: 4987)
MEGSPHSKA,
(SEQ ID NO: 4988)
ENGSPHSKA,
(SEQ ID NO: 4989)
GSASPHSKA,
(SEQ ID NO: 4990)
GNGSPHSKS,
(SEQ ID NO: 4991)
KNGSPHSKA,
(SEQ ID NO: 4992)
KEGSPHSKA,
(SEQ ID NO: 4993)
AIGSPHSKA,
(SEQ ID NO: 4994)
GSGSPHSKN,
(SEQ ID NO: 4995)
GSGSPHAKA,
(SEQ ID NO: 4996)
GHDSPHKIG,
(SEQ ID NO: 4997)
GYDSPHKSG,
(SEQ ID NO: 4998)
GHESPHKSG,
(SEQ ID NO: 4999)
GHDSPHKTG,
(SEQ ID NO: 5000)
GRGSPHKRG,
(SEQ ID NO: 4908)
GQDSPHKSG,
(SEQ ID NO: 5001)
GHDSPHKSL,
(SEQ ID NO: 5002)
GHGSPHSKA,
(SEQ ID NO: 5003)
GHDSPHKSE,
(SEQ ID NO: 4913)
VSGSPHSKA,
(SEQ ID NO: 5004)
GRDSPHKSG,
(SEQ ID NO: 5005)
GNDSPHKSV,
(SEQ ID NO: 5006)
GODSPHKIG,
(SEQ ID NO: 5007)
GHDSPHKSV,
(SEQ ID NO: 5008)
GPDSPHKIG,
(SEQ ID NO: 5009)
GPDSPHKSG,
(SEQ ID NO: 5010)
GHDSPHKSW,
(SEQ ID NO: 5011)
GHDSPHKSN,
(SEQ ID NO: 5012)
GMGSPHSKT,
(SEQ ID NO: 5013)
GHDSPHKHG,
(SEQ ID NO: 5014)
GQVSPHKSG,
(SEQ ID NO: 5015)
GDDSPHKSV,
(SEQ ID NO: 5016)
GHNSPHKSG,
(SEQ ID NO: 5017)
GNGSPHKRG,
(SEQ ID NO: 5018)
GHDSPHKYG,
(SEQ ID NO: 5019)
GHDSPHKSQ,
(SEQ ID NO: 5020)
GNDSPHKIG,
(SEQ ID NO: 5021)
GHDSPHKSK,
(SEQ ID NO: 5022)
GHDSPHKLW,
(SEQ ID NO: 5023)
GHPSPHWKG,
(SEQ ID NO: 5024)
GHDSPHKMG,
(SEQ ID NO: 5025)
GHDSPHKMA,
or
(SEQ ID NO: 5026)
GHSSPHRSG;

(ii) an amino acid sequence comprising any portion of an amino acid sequence in (i), e.g., any 2, 3, 4, 5, 6, 7, or 8 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   20. The AAV particle of any one of embodiments 1-19, wherein [N3] comprises SK, KA, KS, or SG.
   21. The AAV particle of any one of embodiments 1-20, wherein [N3] is or comprises SKA. KSG, or KYG.
   22. The AAV particle of any one of embodiments 1-21, wherein [N2]-[N3] comprises SPHSK (SEQ ID NO: 4701), SPHKS (SEQ ID NO: 4704), or SPHKY (SEQ ID NO: 4715).
   23. The AAV particle of any one of embodiments 1-22, wherein [N2]-[N3] is or comprises SPHSKA (SEQ ID NO: 941).
   24. The AAV particle of any one of embodiments 1-22, wherein [N2]-[N3] is or comprises SPHKSG (SEQ ID NO: 946).
   25. The AAV particle of any one of embodiments 1-22, wherein [N2]-[N3] is or comprises SPHKYG (SEQ ID NO: 966).
   26. The AAV particle of any one of embodiments 1-25, wherein [N1] comprises GS, SG, GH, or HD.
   27. The AAV particle of any one of embodiments 1-26, wherein [N1] is or comprises GSG.
   28. The AAV particle of any one of embodiments 1-26, wherein [N1] is or comprises GHD.
   29. The AAV particle of any one of embodiments 1-23 or 26-27, wherein [N1]-[N2]-[N3] comprises SGSPHSK (SEQ ID NO: 4839).
   30. The AAV particle of any one of embodiments 1-22, 24, 26, or 28, wherein [N1]-[N2]-[N3] comprises HDSPHKS (SEQ ID NO: 4840).
   31. The AAV particle of any one of embodiments 1-22 or 25-27, wherein [N1]-[N2]-[N3] comprises SGSPHKYG (SEQ ID NO: 5027).
   32. The AAV particle of any one of embodiments 1-8, 10, 12-23, 26-27, or 29, wherein [N1]-[N2]-[N3] is or comprises GSGSPHSKA (SEQ ID NO: 4697).
   33. The AAV particle of any one of embodiments 1-9, 11-22, 24, 26, 28, or 30, wherein [N1]-[N2]-[N3] is or comprises GHDSPHKSG (SEQ ID NO: 4698).
   34. The AAV particle of any one of embodiments 1-8, 10, 12-22, 25-27, or 31, wherein [N1]-[N2]-[N3] is or comprises GSGSPHKYG (SEQ ID NO: 4927).
   35. The AAV particle of any one of embodiments 1-34, wherein [N1]-[N2]-[N3] replaces positions 453-455, numbered according to SEQ ID NO: 138.
   36. The AAV particle of any one of embodiments 1-35, wherein the AAV capsid variant comprises an amino acid other than Q at position 456 (e.g., W, K, R, G, L, V, S, P, H, K, I, M, A, E, or F), an amino acid other than N at position 457 (e.g., Y, C, K, T, H, R, D, V, S, P, G, W, E, F, A, I, M, Q, or L), an amino acid other than Q at position 458 (e.g., G, K, H, R, T, L, D, A, P, I, F, V, M, W, Y, S, E, N, or Y), and/or an amino acid other than Q at position 459 (e.g., H, L, R, W, K, A, P, E, M, 1, S, G, N, Y, C, V, T, D, or V), numbered according to SEQ ID NO: 138.
   37. The AAV particle of any one of embodiments 1-36, wherein the AAV capsid variant comprises an amino acid other than Q at position 462 (e.g., W, K, R, G, L, V, S, P, H, K, I, M, A, E, or F), an amino acid other than N at position 463 (e.g., Y, C, K, T, H, R, D, V, S, P, G, W, E, F, A, I, M, Q, or L), an amino acid other than Q at position 464 (e.g., G, K, H, R, T, L, D, A, P, I, F, V, M, W, Y, S, E, N, or Y), and/or an amino acid other than Q at position 465 (e.g., H, L, R, W, K, A, P, E, M, I, S, G, N, Y, C, V, T, D, or V), numbered according to SEQ ID NO: 981, 982, 36, 37, 39, 40,42-46, 48, 49, 50, 52, 53, 56, or 57.
   38. The AAV particle of any one of embodiments 1-37, wherein the AAV capsid variant comprises:
(a) the amino acid Q at position 456, the amino acid N at position 457, the amino acid Q at position 458, and/or the amino acid Q at position 459, numbered according to SEQ ID NO: 138; or
(b) the amino acid Q at position 462, the amino acid N at position 463, the amino acid Q at position 464, and/or the amino acid Q at position 465, numbered according to SEQ ID NO: 981, 982, 36, 37, 39, 40, 42-46, 48, 49, 50, 52, 53, 56, or 57.
   39. The AAV particle of any one of embodiments 1-38, wherein the AAV capsid variant further comprises [N1], wherein [N4] comprises X7 X8 X9 X10, and wherein:
(a) X7 is: Q, W, K, R, G, L, V, S, P, H, K, I, M, A, E, or F;
(b) X8 is: N, Y, C, K, T, H, R, D, V, S, P, G, W, E, F, A, I, M, Q, or L;
(c) X9 is: Q, G, K, H, R, T, L, D, A, P, I, F, V, M, W, Y, S, E, N, or Y; and
(d) X10 is: Q, H, L, R, W, K, A, P, E, M, I, S, G, N, Y, C, V, T, D, or V;
optionally wherein the AAV capsid variant comprises an amino acid modification, e.g., a conservative substitution, of any of the aforesaid amino acids in (a)-(d).
   40. The AAV particle of embodiment 39, wherein:
(a) X7 of [N4] is Q or R;
(b) X8 of [N4] is N or R;
(c) X9 of [N4] is Q or R; and
(d) X10 of [N4] is Q, L, or R.

41. The AAV particle of embodiment 39 or 40, wherein [N4] is or comprises:

(i)
(SEQ ID NO: 5028)
QNQQ,
(SEQ ID NO: 5029)
WNQQ,
(SEQ ID NO: 5030)
QYYV,
(SEQ ID NO: 5031)
RRQQ,
(SEQ ID NO: 5032)
GCGQ,
(SEQ ID NO: 5033)
LRQQ,
(SEQ ID NO: 5034)
RNQQ,
(SEQ ID NO: 5035)
VNQQ,
(SEQ ID NO: 5036)
FRLQ,
(SEQ ID NO: 5037)
FNQQ,
(SEQ ID NO: 5038)
LLQQ,
(SEQ ID NO: 5039)
SNQQ,
(SEQ ID NO: 5040)
RLQQ,
(SEQ ID NO: 5041)
LNQQ,
(SEQ ID NO: 5042)
QRKL,
(SEQ ID NO: 5043)
LRRQ,
(SEQ ID NO: 5044)
QRLR,
(SEQ ID NO: 5045)
QRRL,
(SEQ ID NO: 5046)
RRLQ,
(SEQ ID NO: 5047)
RLRQ,
(SEQ ID NO: 5048)
SKRQ,
(SEQ ID NO: 5049)
QLYR,
(SEQ ID NO: 5050)
QLTV,
(SEQ ID NO: 5051)
QNKQ,
(SEQ ID NO: 5052)
KNQQ,
(SEQ ID NO: 5053)
QKQQ,
(SEQ ID NO: 5054)
QTQQ,
(SEQ ID NO: 5055)
QNHQ,
(SEQ ID NO: 5056)
QHQQ,
(SEQ ID NO: 5057)
QNQH,
(SEQ ID NO: 5058)
QHRQ,
(SEQ ID NO: 5059)
LTQQ,
(SEQ ID NO: 5060)
QNQW,
(SEQ ID NO: 5061)
QNTH,
(SEQ ID NO: 5062)
RRRQ,
(SEQ ID NO: 5063)
QYQQ,
(SEQ ID NO: 5064)
QNDQ,
(SEQ ID NO: 5065)
QNRH,
(SEQ ID NO: 5066)
RDQQ,
(SEQ ID NO: 5067)
PNLQ,
(SEQ ID NO: 5068)
HVRQ,
(SEQ ID NO: 5069)
PNQH,
(SEQ ID NO: 5070)
HNQQ,
(SEQ ID NO: 5071)
QSQQ,
(SEQ ID NO: 5072)
QPAK,
(SEQ ID NO: 5073)
QNLA,
(SEQ ID NO: 5074)
QNQL,
(SEQ ID NO: 5075)
QGQQ,
(SEQ ID NO: 5076)
LNRQ,
(SEQ ID NO: 5077)
QNPP,
(SEQ ID NO: 5078)
QNLQ,
(SEQ ID NO: 5079)
QDQE,
(SEQ ID NO: 5080)
QDQQ,
(SEQ ID NO: 5081)
HWQQ,
(SEQ ID NO: 5082)
PNQQ,
(SEQ ID NO: 5083)
PEQQ,
(SEQ ID NO: 5084)
QRTM,
(SEQ ID NO: 5085)
LHQH,
(SEQ ID NO: 5086)
QHRI,
(SEQ ID NO: 5087)
QYIH,
(SEQ ID NO: 5088)
QKFE,
(SEQ ID NO: 5089)
QFPS,
(SEQ ID NO: 5090)
QNPL,
(SEQ ID NO: 5091)
QAIK,
(SEQ ID NO: 5092)
QNRQ,
(SEQ ID NO: 5093)
QYQH,
(SEQ ID NO: 5094)
QNPQ,
(SEQ ID NO: 5095)
QHQL,
(SEQ ID NO: 5096)
QSPP,
(SEQ ID NO: 5097)
QAKL,
(SEQ ID NO: 5098)
KSQQ,
(SEQ ID NO: 5099)
QDRP,
(SEQ ID NO: 5100)
QNLG,
(SEQ ID NO: 5101)
QAFH,
(SEQ ID NO: 5102)
QNAQ,
(SEQ ID NO: 5103)
HNQL,
(SEQ ID NO: 5104)
QKLN,
(SEQ ID NO: 5105)
QNVQ,
(SEQ ID NO: 5106)
QAQQ,
(SEQ ID NO: 5107)
QTPP,
(SEQ ID NO: 5108)
QPPA,
(SEQ ID NO: 5109)
QERP,
(SEQ ID NO: 5110)
QDLQ,
(SEQ ID NO: 5111)
QAMH,
(SEQ ID NO: 5112)
QHPS,
(SEQ ID NO: 5113)
PGLQ,
(SEQ ID NO: 5114)
QGIR,
(SEQ ID NO: 5115)
QAPA,
(SEQ ID NO: 5116)
QIPP,
(SEQ ID NO: 5117)
QTQL,
(SEQ ID NO: 5118)
QAPS,
(SEQ ID NO: 5119)
QNTY,
(SEQ ID NO: 5120)
QDKQ,
(SEQ ID NO: 5121)
QNHL,
(SEQ ID NO: 5122)
QIGM,
(SEQ ID NO: 5123)
LNKQ,
(SEQ ID NO: 5124)
PNQL,
(SEQ ID NO: 5125)
QLQQ,
(SEQ ID NO: 5126)
QRMS,
(SEQ ID NO: 5127)
QGIL,
(SEQ ID NO: 5128)
QDRQ,
(SEQ ID NO: 5129)
RDWQ,
(SEQ ID NO: 5130)
QERS,
(SEQ ID NO: 5131)
QNYQ,
(SEQ ID NO: 5132)
QRTC,
(SEQ ID NO: 5133)
QIGH,
(SEQ ID NO: 5134)
QGAI,
(SEQ ID NO: 5135)
QVPP,
(SEQ ID NO: 5136)
QVQQ,
(SEQ ID NO: 5137)
LMRQ,
(SEQ ID NO: 5138)
QYSV,
(SEQ ID NO: 5139)
QAIT,
(SEQ ID NO: 5140)
QKTL,
(SEQ ID NO: 5141)
QLHH,
(SEQ ID NO: 5142)
QNII,
(SEQ ID NO: 5143)
QGHH,
(SEQ ID NO: 5144)
QSKV,
(SEQ ID NO: 5145)
QLPS,
(SEQ ID NO: 5146)
IGKQ,
(SEQ ID NO: 5147)
QAIH,
(SEQ ID NO: 5148)
QHGL,
(SEQ ID NO: 5149)
QFMC,
(SEQ ID NO: 5150)
QNQM,
(SEQ ID NO: 5151)
QHLQ,
(SEQ ID NO: 5152)
QPAR,
(SEQ ID NO: 5153)
QSLQ,
(SEQ ID NO: 5154)
QSQL,
(SEQ ID NO: 5155)
HSQQ,
(SEQ ID NO: 5156)
QMPS,
(SEQ ID NO: 5157)
QGSL,
(SEQ ID NO: 5158)
QVPA,
(SEQ ID NO: 5159)
HYQQ,
(SEQ ID NO: 5160)
QVPS,
(SEQ ID NO: 5161)
RGEQ,
(SEQ ID NO: 5162)
PGQQ,
(SEQ ID NO: 5163)
LEQQ,
(SEQ ID NO: 5164)
QNQS,
(SEQ ID NO: 5165)
QKVI,
(SEQ ID NO: 5166)
QNND,
(SEQ ID NO: 5167)
QSVH,
(SEQ ID NO: 5168)
QPLG,
(SEQ ID NO: 5169)
HNQE,
(SEQ ID NO: 5170)
QIQQ,
(SEQ ID NO: 5171)
QVRN,
(SEQ ID NO: 5172)
PSNQ,
(SEQ ID NO: 5173)
QVGH,
(SEQ ID NO: 5174)
QRDI,
(SEQ ID NO: 5175)
QMPN,
(SEQ ID NO: 5176)
RGLQ,
(SEQ ID NO: 5177)
PSLQ,
(SEQ ID NO: 5178)
QRDQ,
(SEQ ID NO: 5179)
QAKG,
(SEQ ID NO: 5180)
QSAH,
(SEQ ID NO: 5181)
QSTM,
(SEQ ID NO: 5182)
QREM,
(SEQ ID NO: 5183)
QYRA,
(SEQ ID NO: 5184)
QRQQ,
(SEQ ID NO: 5185)
QWQQ,
(SEQ ID NO: 5186)
QRMN,
(SEQ ID NO: 5187)
GDSQ,
(SEQ ID NO: 5188)
QKIS,
(SEQ ID NO: 5189)
PSMQ,
(SEQ ID NO: 5190)
SPRQ,
(SEQ ID NO: 5191)
MEQQ,
(SEQ ID NO: 5192)
QYQN,
(SEQ ID NO: 5193)
QIRQ,
(SEQ ID NO: 5194)
QSVQ,
(SEQ ID NO: 5195)
RSQQ,
(SEQ ID NO: 5196)
QNKL,
(SEQ ID NO: 5197)
QIQH,
(SEQ ID NO: 5198)
PRQQ,
(SEQ ID NO: 5199)
HTQQ,
(SEQ ID NO: 5200)
QRQH,
(SEQ ID NO: 5201)
RNQE,
(SEQ ID NO: 5202)
QSKQ,
(SEQ ID NO: 5203)
QNQP,
(SEQ ID NO: 5204)
QSPQ,
(SEQ ID NO: 5205)
QTRQ,
(SEQ ID NO: 5206)
QNLH,
(SEQ ID NO: 5207)
QNQE,
(SEQ ID NO: 5208)
LNQP,
(SEQ ID NO: 5209)
QNQD,
(SEQ ID NO: 5210)
QNLL,
(SEQ ID NO: 5211)
QLVI,
(SEQ ID NO: 5212)
RTQE,
(SEQ ID NO: 5213)
QTHQ,
(SEQ ID NO: 5214)
QDQH,
(SEQ ID NO: 5215)
QSQH,
(SEQ ID NO: 5216)
VRQQ,
(SEQ ID NO: 5217)
AWQQ,
(SEQ ID NO: 5218)
QSVP,
(SEQ ID NO: 5219)
QNIQ,
(SEQ ID NO: 5220)
LDQQ,
(SEQ ID NO: 5221)
PDQQ,
(SEQ ID NO: 5222)
ESQQ,
(SEQ ID NO: 5223)
QRQL,
(SEQ ID NO: 5224)
QIV,
(SEQ ID NO: 5225)
QKQS,
(SEQ ID NO: 5226)
QSHQ,
(SEQ ID NO: 5227)
QFVV,
(SEQ ID NO: 5228)
QSQP,
(SEQ ID NO: 5229)
QNEQ,
(SEQ ID NO: 5230)
INQQ,
(SEQ ID NO: 5231)
RNRQ,
(SEQ ID NO: 5232)
RDQK,
(SEQ ID NO: 5233)
QWKR,
(SEQ ID NO: 5234)
ENRQ,
(SEQ ID NO: 5235)
QTQP,
(SEQ ID NO: 5236)
QKQL,
(SEQ ID NO: 5237)
RNQL,
(SEQ ID NO: 5238)
ISIQ,
(SEQ ID NO: 5239)
QTVC,
(SEQ ID NO: 5240)
QQIM,
(SEQ ID NO: 5241)
LNHQ,
(SEQ ID NO: 5242)
QNQA,
(SEQ ID NO: 5243)
QMIH,
(SEQ ID NO: 5244)
RNHQ,
or
(SEQ ID NO: 5245)
QKMN;

(ii) an amino acid sequence comprising any portion of an amino acid sequence in (i), e.g., any 2, or 3 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   42. The AAV particle of any one of embodiments 39-41, wherein [N1]-[N2]-[N3]-[N4] is or comprises:
(i) the amino acid sequence of any of SEQ ID NOs: 1800-2241:
(ii) an amino acid sequence comprising any portion of an amino acid sequence in (i), e.g., any 2, 3, 4, 5, 6, 7, 8, 9, 10, 11, or 12 amino acids, e.g., consecutive amino acids, thereof;
(iii) an amino acid sequence comprising one, two, or three but no more than four modifications relative to any of the amino acid sequences in (i): or
(iv) an amino acid sequence comprising one, two, or three but no more than four different amino acids, relative to any one of the amino acid sequences in (i).
   43. The AAV particle of am one of embodiments 39-42, wherein [N1]-[N2]-[N3]-[N4] is or comprises GSGSPHSKAQNQQ (SEQ ID NO: 1801).
   44. The AAV particle of any one of embodiments 39-42, wherein [N1]-[N2]-[N3]-[N4] is or comprises GHDSPHKSGQNQQ (SEQ ID NO: 1800).
   45. The AAV particle of any one of embodiments 3942, wherein [N1]-[N2]-[N3]-[N4] is or comprises GSGSPHKYGQNQQT (SEQ ID NO: 910).
   46. The AAV particle of any one of embodiments 145, wherein the AAV capsid variant comprises an amino acid other than T at position 450 (e.g., S, Y, M, A, C, I, R, L, D, F, V, Q, N, H, E, or G), an amino acid other than I at position 451 (e.g., M, P, E, N, D, S, A, T, G, Q, F, V, L, C, H, R, W, or L), and/or an amino acid other than N at position 452 (e.g., M, E, G, Y, W, T, I, Q, F, V, A, L, I, P, K, R, H, S, D, or S), numbered according to any one of SEQ ID NOs: 36-59, 138, 981, or 982.
   47. The AAV particle of any one of embodiments 1-46, wherein the AAV capsid variant comprises the amino acid T at position 450, the amino acid I at position 451, and/or the amino acid N at position 452, numbered according to any one of SEQ ID NOs: 138, 981, or 982.
   48. The AAV particle of any one of embodiments 1-47, wherein the AAV capsid variant further comprises [N0], wherein [N0] comprises X_{A} X_{B} and X_{C}, and wherein:
(a) X_{A} is: T, S, Y, M, A, C, 1, R, L, D, F, V, Q, N, H, E, or G;
(b) X_{B} is: 1, M, P, E, N, D, S, A, T, G, Q, F, V, L, C, H, R, W, or L; and
(c) X_{C} is: N, M, E, G, Y, W,
optionally wherein the AAV capsid variant comprises an amino acid modification, e.g., a conservative substitution, of any of the aforesaid amino acids in (a)-(c).
   49. The AAV particle of embodiment 48, wherein [N0] is or comprises TIN, SMN, TIM, YLS, GLS, MPE, MEG, MEY, AEW, CEW, ANN, IPE, ADM, IEY, ADY, IET, MEW, CEY, RIN, MEI, LEY, ADW, IEI, DIM, FEQ, MEF, CDQ, LPE, IEN, MES, AEI, VEY, IIN, TSN, IEV, MEM, AEV, MDA, VEW, AEQ, LEW, MEL, MET, MEA, IES, MEV, CEI, ATN, MDG, QEV, ADQ, NMN, IEM, ISN, TGN, QQQ, HDW, LEG, TI1, TFP, TEK, EIN, TVN, TFN, SIN, TER, TSY, ELH, AlN, SVN, TDN, TFH, TVH, TEN, TSS, TID, TCN, NIN, TEH, AEM, AIK, TDK, TFK, SDQ, TEI, NTN, TET, SIK, TEL, TEA, TAN, TIY, TFS, TES, TTN, TED, TNN, EVH, TIS, TVR, TDR, TIK, NHI, TIP, ESD, TDL, TVP, TVI, AEH, NCL, TVK, NAD, TIT, NCV, TIR, NAL, VIN, TIQ, TEF, TRE, QGE, SEK, NVN, GGE, EFV, SDK, TEQ, EVQ, TEY, NCW, TDV, SDI, NSI, NSL, EVV, TEP, SEL, TWQ, TEV, AVN, GVL, TLN, TEG, TRD, NAL, AEN, AET, ETA, NNL, or any dipeptide thereof.
   50. The AAV particle of embodiment 48 or 49, wherein [N0]-[N1]-[N2]-[N3]-[N4] is or comprises:
(i) the amino acid sequence of any one of SEQ ID NOs: 2242-2886;
(ii) an amino acid sequence comprising any portion of an amino acid sequence in (i), e.g., any 2, 3, 4, 5, 6, 7, 8, 9, 10, 11, 12, 13, 14, or 15 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   51. The AAV particle of any one of embodiments 48-50, wherein [N1]-[N1]-[N2]-[N3]-[N4] is or comprises TINGSGSPHSKAQNQQ (SEQ ID NO: 2242).
   52. The AAV particle of any one of embodiments 48-50, wherein [N0]-[N1]-[N2]-[N3]-[N4] is or comprises TINGHDSPHKSGQNQQ (SEQ ID NO: 2243).
   53. The AAV particle of any one of embodiments 48-52, wherein [N0]-[N1]-[N2]-[N3]-[N4] is or comprises TINGSGSPHKYGQNQQT (SEQ ID NO: 5246).
   54. The AAV particle of any one of embodiments 1-53, wherein [N1]-[N2]-[N3] is present in loop IV.
   55. The AAV particle of any one of embodiments 48-54, wherein [N0] and [N4] are present in loop IV.
   56. The AAV particle of any one of embodiments 48-55, wherein [N0] is present immediately subsequent to position 449, numbered according to SEQ ID NO: 138.
   57. The AAV particle of any one of embodiments 48-56, wherein [N0] is present immediately subsequent to position 449, numbered according to any one of SEQ ID NOs: 36-59, 981, or 982.
   58. The AAV particle of any one of embodiments 48-57, wherein [N0] replaces positions 450, 451, and 452 (e.g., T450.1451, and N452), numbered according to SEQ ID NO: 138.
   59. The AAV particle of any one of embodiments 48-58, wherein [N0] replaces positions 450-452 (e.g., T450, 1451, and N452), numbered according to any one of SEQ ID NOs: 36-59, 981, or 982.
   60. The AAV particle of any one of embodiments 48-59, wherein [N0] corresponds to positions 450-452 of any one of SEQ ID NOs: 36-59, 138, 981 or 982.
   61. The AAV particle of any one of embodiments 48-60, wherein [N0] is present immediately subsequent to position 449 and wherein [N0] replaces positions 450452 (e.g., T450, 1451, and N452), numbered according to SEQ ID NO: 138.
   62. The AAV particle of any one of embodiments 48-61, wherein [N0] is present immediately subsequent to position 449 and wherein [N0] replaces positions 450452 (e.g., T450, 1451, and N452), numbered according to any one of SEQ ID NOs: 36-59, 981, or 982.
   63. The AAV particle of any one of embodiments 1-62, wherein [N1] is present immediately subsequent to position 452, numbered according to the amino acid sequence of SEQ ID NO: 138.
   64. The AAV particle of any one of embodiments 1-63, wherein [N1] is present immediately subsequent to position 452, numbered according to SEQ ID NO: 981 or 982.
   65. The AAV particle of any one of embodiments 1-61, wherein [N1] replaces positions 453455 (e.g., G453, S454, and G455), numbered according to SEQ ID NO: 138.
   66. The AAV particle of any one of embodiments 1-64, wherein [N1] replaces positions 453 (e.g., G453), numbered according to SEQ ID NO: 138.
   67. The AAV particle of any one of embodiments 1-65, wherein [N1] replaces positions 453455 (e.g., G453, S454, and G455), numbered according to SEQ ID NO: 981.
   68. The AAV particle of any one of embodiments 1-65 or 67, wherein [N1] replaces positions 453-455, numbered according to SEQ ID NO: 982.
   69. The AAV particle of any one of embodiments 1-65, 67, or 68, wherein [N1] is present immediately subsequent to position 452 and wherein [N1] replaces positions 453455 (e.g., G453, S454, and G455), numbered according to SEQ ID NO: 138.
   70. The AAV particle of any one of embodiments 1-64 or 66, wherein [N1] is present immediately subsequent to position 452 and wherein [N1] replaces positions 453 (e.g., G453), numbered according to SEQ ID NO: 138.
   71. The AAV particle of any one of embodiments 1-64.66 or 70, wherein [N1] is present immediately subsequent to position 452 and wherein [N1] replaces positions 453-455, numbered according to SEQ ID NO: 4, 36, 981, or 982.
   72. The AAV particle of any one of embodiments 1-71, wherein [N1] corresponds to positions 453-455, numbered according to any one of SEQ ID NOs: 4, 36-59, 981, or 982.
   73. The AAV particle of any one of embodiments 1-72, wherein the AAV capsid variant comprises an amino acid other than S at position 454 and/or an amino acid other than G at position 455, numbered according to SEQ ID NO: 138, 981, or 982.
   74. The AAV particle of any one of embodiments 1-73, wherein the AAV capsid variant comprises the amino acid H at position 454 and the amino acid D at position 455, numbered according to SEQ ID NO: 138 or 982.
   75. The AAV particle of any one of embodiments 1-74, wherein the AAV capsid variant comprises a substitution at position 454 (e.g., S454H) and/or a substitution at position 455 (e.g., G455D), numbered according to SEQ ID NO: 138.
   76. The AAV particle of any one of embodiments 1-75, wherein the AAV capsid variant comprises the amino acid H at position 454 and the amino acid D at position 455, and further comprises the amino acid sequence SPHSKA (SEQ ID NO: 941) immediately subsequent to position 455, numbered according to SEQ ID NO: 138.
   77. The AAV particle of any one of embodiments 1-76, wherein the AAV capsid variant comprises the amino acid H at position 454 and the amino acid D at position 455, numbered according to SEQ ID NO: 982.
   78. The AAV particle of any one of embodiments 1-77, wherein the AAV capsid variant comprises the amino acid H at position 454 and the amino acid D at position 455, and further comprises the amino acid sequence SPHSKA (SEQ ID NO: 941) immediately subsequent to position 455, numbered according to SEQ ID NO: 982.
   79. The AAV particle of any one of embodiments 1-72, wherein the AAV capsid variant comprises the amino acid S at position 454 and the amino acid G at position 455, numbered according to SEQ ID NO: 138.
   80. The AAV particle of any one of embodiments 1-72 or 79, wherein the AAV capsid variant comprises the amino acid S at position 454 and the amino acid G at position 455, and further comprises the amino acid sequence SPHSKA (SEQ ID NO: 941) immediately subsequent to position 455, numbered according to SEQ ID NO: 138.
   81. The AAV particle of any one of embodiments 1-72, 79, or 80, wherein the AAV capsid variant comprises the amino acid S at position 454 and the amino acid G at position 455, numbered according to SEQ ID NO: 981.
   82. The AAV particle of any one of embodiments 1-72 or 79-81, wherein the AAV capsid variant comprises the amino acid S at position 454 and the amino acid G at position 455, and further comprises the amino acid sequence SPHSKA (SEQ ID NO: 941) immediately subsequent to position 455, numbered according to SEQ ID NO: 981.
   83. The AAV particle of any one of embodiments 1-82, wherein [N2] is present immediately subsequent to position 455, numbered according to SEQ ID NO: 138.
   84. The AAV particle of any one of embodiments 1-83, wherein [N2] corresponds to positions 456-458 (e.g., S456. P457, H458) of SEQ ID NO: 981 or 982.
   85. The AAV particle of any one of embodiments 1-83, wherein [N2] corresponds to positions 456-458 (e.g., S456, P457, H458) of any one of SEQ ID NOs: 4 or 36-59.
   86. The AAV particle of any one of embodiments 1-85, wherein [N2]-[N3] is present immediately subsequent to position 455, numbered according to SEQ ID NO: 138.
   87. The AAV particle of any one of embodiments 1-86, wherein [N2] is present immediately subsequent to position 455, numbered according to SEQ ID NO: 4, 36, 981, or 982.
   88. The AAV particle of any one of embodiments 1-87, wherein [N2]-[N3] is present immediately subsequent to position 455, numbered according to SEQ ID NO: 4, 36, 981, or 982.
   89. The AAV particle of any one of embodiments 1-88, wherein [N2]-[N3] corresponds to positions 456-461 (e.g., S456, P457, H458, S459, K460, A461) of SEQ ID NO: 981.
   90. The AAV particle of any one of embodiments 1-88, wherein [N2]-[N3] corresponds to positions 456-461 (e.g., S456, P457, H458, K459, S460, G461) of SEQ ID NO: 982.
   91. The AAV particle of any one of embodiments 1-90, wherein [N2] is present immediately subsequent to [N1].
   92. The AAV particle of any one of embodiments 1-64, 66, 70, or 71, wherein [N3] is present immediately subsequent to [N2] and replaces positions 454 and 455 (e.g., S454 and G455), numbered according to SEQ ID NO: 138.
   93. The AAV particle of any one of embodiments 1-1-64, 66, 70, 71, or 92, wherein [N3] is present immediately subsequent to [N1]-[N2] and replaces positions 454 and 455 (e.g., S454 and G455), numbered according to SEQ ID NO: 138.
   94. The AAV particle of any one of embodiments 39-93, wherein [N4] is present immediately subsequent to position 455, numbered according to SEQ ID NO: 138.
   95. The AAV particle of any one of embodiments 39-94, wherein [N4] replaces positions 456-459 (e.g., Q456, N457, Q458, and Q459), numbered according to SEQ ID NO: 138.
   96. The AAV particle of any one of embodiments 39-95, wherein [N4] corresponds to positions 462-465 (e.g., Q462, N463, Q464, Q465) of SEQ ID NO: 4, 36, 981, or 982.
   97. The AAV particle of any one of embodiments 39-96, wherein [N2]-[N3]-[N4] replaces positions 456459 (e.g., Q456, N457, Q458, and Q459), numbered according to SEQ ID NO: 138.
   98. The AAV particle of any one of embodiments 39-97, wherein [N2]-[N3]-[N4] is present immediately subsequent to position 455, and wherein [N2]-[N3]-[N4] replaces positions 456-459 (e.g., Q456, N457, Q458, and Q459), numbered according to SEQ ID NO: 138.
   99. The AAV particle of any one of embodiments 39-98, wherein [N2]-[N3]-[N4] corresponds to positions 456-465 (e.g., S456, P457, H458, S459, K460. A461, Q462, N463, Q464, Q465) of SEQ ID NO: 981.
   100. The AAV particle of any one of embodiments 39-98, wherein [N2]-[N3]-[N4] corresponds to positions 456-465 (e.g., S456, P457, H458, K459, S460, G461, Q462, N463, Q464, Q465) of SEQ ID NO: 982.
   101. The AAV particle of any one of embodiments 39-98, wherein [N2]-[N3]-[N4] corresponds to positions 456-465 of any one of SEQ ID NOs: 4 or 36-59.
   102. The AAV particle of any one of embodiments 39-101, wherein [N1]-[N2]-[N3]-[N4] replaces positions 453-459 (e.g., G453, S454, G455, Q456, N457, Q458, and Q459), numbered according to SEQ ID NO: 138.
   103. The AAV particle of any one of embodiments 39-102, wherein [N1]-[N2]-[N3]-[N4] is present immediately subsequent to position 452, and wherein [N1]-[N2]-[N3]-[N4] replaces positions 453-459 (e.g., G453, S454, G455, Q456, N457, Q458, and Q459), numbened according to SEQ ID NO: 138.
   104. The AAV particle of any one of embodiments 39-99, 102, or 103, wherein INIl-[N2]-[N3]-[N4] corresponds to positions 453-465 (e.g., G453, 5454, G455, S456, P457, H458, S459, K460, A461. Q462, N463, Q464, Q465) of SEQ ID NO: 981.
   105. The AAV particle of any one of embodiments 39-98, 100, 102, or 103, wherein [N1]-[N2]-[N3]-[N4] corresponds to positions 453-465 (e.g., G453, H454, D455, 5456, P457, H458, K459, S460, G461, Q462, N463, Q464, Q465) of SEQ ID NO: 982.
   106. The AAV particle of any one of embodiments 39-98, 102, or 103, wherein [N1]-[N2]-[N3]-[N4] corresponds to positions 453465 of any one of SEQ ID NOs: 4 or 36-59.
   107. The AAV particle of any one of embodiments 1-99 or 102-104, wherein [N1]-[N2]-[N3] corresponds to positions 453461 (e.g., G453, S454, G455, S456, P457, H458, S459, K460, A461) of SEQ ID NO: 981.
   108. The AAV particle of any one of embodiments 1-98, 100, 102, 103, or 105, wherein [N1]-[N2]-[N3] corresponds to positions 453-461 (e.g., G453, H454, D455, S456, P457, H458, K459, S460, G461) of SEQ ID NO: 982.
   109. The AAV particle of any one of embodiments 39-98, 102, 103, or 106, wherein [N1]-[N2]-[N3] corresponds to positions 453-461 of any one of SEQ ID NOs: 36-59.
   110. The AAV particle of any one of embodiments 48-109, wherein [N0]-[N1]-[N2]-[N3]-[N4] replaces positions 450-459 (e.g., T450, 1451, N452, G453, S454, G455, Q456, N457, Q458, and Q459), numbered according to SEQ ID NO: 138.
   1l1. The AAV particle of any one of embodiments 48-110, wherein [N0]-[N1]-[N2]-[N3]-[N4] is present immediately subsequent to position 449, and wherein [N0]-[N1]-[N2]-[N3]-[N4] replaces positions 450-459 (e.g., T450, 1451, N452, G453, S454, G455, Q456, N457, Q458, and Q459), numbered according to SEQ ID NO: 138.
   112. The AAV particle of any one of embodiments 48-99, 102-104, or 106, wherein [N0]-[N1]-[N2]-[N3]-[N4] corresponds to positions 450-465 (e.g., T450, 1451, N452, G453, S454, G455, S456, P457, H458, S459, K460, A461, Q462, N463, Q464, Q465) of SEQ ID NO: 981.
   113. The AAV particle of any one of embodiments 48-98, 100, 102, 103, 105, or 108, wherein [N0]-[N1]-[N2]-[N3]-[N4] corresponds to positions 450-465 (e.g., T450, 1451, N452, G453, H454, D455, S456, P457, H458, K459, S460, G461, Q462, N463, Q464, Q465) of SEQ ID NO: 982.
   114. The AAV particle of any one of embodiments 48-98, 102, 103, 106, or 109, wherein [N0]-[N1]-[N2]-[N3]-[N4] corresponds to positions 450-465 of any one of SEQ ID NOs: 36-59.
   115. The AAV particle of any one of embodiments 39-114, wherein [N4] replaces positions 462-465 (e.g., Q462, N463, Q464, and Q465), numbered according to SEQ ID NO: 4, 36, 981, or 982.
   116. The AAV particle of any one of embodiments 39-115, wherein [N2]-[N3]-[N4] replaces positions 462465 (e.g., Q462, N463, Q464, and Q465), numbered according to SEQ ID NO: 4, 36, 981, or 982.
   117. The AAV particle of any one of embodiments 39-116, wherein [N2]-[N3]-[N4] is present immediately subsequent to position 455, and wherein [N2]-[N3]-[N4] replaces positions 462465 (e.g., Q462, N463, Q464, and Q465), numbered according to SEQ ID NO: 4, 36, 981, or 982.
   118. The AAV particle of an one of embodiments 1-117, wherein [N3] is present immediately subsequent to [N2].
   119. The AAV particle of any one of embodiments 1-118, wherein the AAV capsid variant comprises, from N-terminus to C-terminus, [N2]-[N3].
   120. The AAV particle of any one of embodiments 1-119, wherein the AAV capsid variant comprises, from N-terminus to C-terminus, [N1]-[N2]-[N31.
   121. The AAV particle of any one of embodiments 48-120, wherein the AAV capsid variant comprises, from N-terminus to C-terminus, [N0]-[N1]-[N2]-[N3].
   122. The AAV particle of any one of embodiments 39-121, wherein the AAV capsid variant comprises, from N-terminus to C-terminus, [N1]-[N2]-[N3]-[N4].
   123. The AAV particle of any one of embodiments 48-122, wherein the AAV capsid variant comprises, from N-terminus to C-terminus, [N0]-[N1]-[N2]-[N3]-[N41.
   124. The AAV particle of any one of the preceding embodiments, wherein the AAV capsid variant comprises an amino acid other T at position 460 (e.g., N, 1, C, H, R, L, D, Y, A, M, Q, I, E, K, P, G or S), numbered according to SEQ ID NO: 138.
   125. The AAV particle of any one of the preceding embodiments, wherein the AAV capsid variant comprises the amino acid N, I, C, H, R, L, D, Y, A, M, Q, I, E, K, P, G or S at position 460, numbered according to SEQ ID NO: 138.
   126. The AAV particle of any one of the preceding embodiments, wherein the AAV capsid variant comprises an amino acid other T at position 466 (e.g., N, I, C, H, R, L, D, Y, A, M, Q, I, E, K, P, G or S), numbered according to any one of SEQ ID NOs: 36-59, 981, or 982.
   127. The AAV particle of any one of the preceding embodiments, wherein the AAV capsid variant comprises the amino acid N, 1, C, H, R, L, D, Y, A, M, Q, I, E, K, P, G or S at position 466, numbered according to any one of SEQ ID NOs: 36-59, 981 or 982.
   128. The AAV particle of any one of the preceding embodiments, wherein the AAV capsid variant comprises an amino acid other K at position 449 (e.g., an E, an N, or a T), numbered according to any one of SEQ ID NOs: 36-59, 138, 981, or 982.
   129. The AAV particle of any one of the preceding embodiments, wherein the AAV capsid variant comprises the amino E, N, or T at position 449, numbered according to any one of SEQ ID NOs: 36-59, 138, 981 or 982.
   130. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises [A][B](SEQ ID NO: 4694), wherein:
(i) [A] comprises the amino acid sequence of GSGSPH (SEQ ID NO: 4695); and
(ii) [B] comprises X1 X2 X3 X4 X5 X6 X7, wherein:
   (a) X1 is: S, C, F, or V;
   (b) X2 is: K, L, R, I, E, Y, V, or S;
   (c) X3 is: A, R, L, G, I, Y, S, F, or W;
   (d) X4 is: W, Q, R, G, L, V, S, or F;
   (e) X5 is: N, Y, R, C, K, or L;
   (f) X6 is: Q, G, K, R, T, L, or Y; and
   (g) X7 is: Q, L, R, or V:
optionally wherein the AAV capsid variant comprises an amino acid modification, e.g., a conservative substitution, of any of the aforesaid amino acids in (a)-(g).
   131. The AAV particle of embodiment 130, wherein
(a) X1 is S;
(b) X2 is K or L;
(c) X3 is: A, R, or L;
(d) X4 is: Q or R;
(e) X5 is: N or R;
(f) X6 is: Q or R; and
(g) X7 is: Q, L, or R. 132. The AAV particle of embodiment 130 or 131, wherein [B] comprises:

(i)
(SEQ ID NO: 5247)
SLLWNQQ,
(SEQ ID NO: 5248)
SKAQYYV,
(SEQ ID NO: 5249)
SKLRRQQ,
(SEQ ID NO: 5250)
SIWQNQQ,
(SEQ ID NO: 5251)
SKAGCGQ,
(SEQ ID NO: 5252)
SRAQNQQ,
(SEQ ID NO: 5253)
SKRLRQQ,
(SEQ ID NO: 5254)
SLRRNQQ,
(SEQ ID NO: 5255)
SRGRNQQ,
(SEQ ID NO: 5256)
SEIVNQQ,
(SEQ ID NO: 5257)
SSRRNQQ,
(SEQ ID NO: 5258)
CLLQNQQ,
(SEQ ID NO: 5259)
SKAFRLQ,
(SEQ ID NO: 5260)
CLAQNQQ,
(SEQ ID) NO: 5261)
FLRQNQQ,
(SEQ ID NO: 5262)
SLRFNQQ,
(SEQ ID NO: 5263)
SYLRNQQ,
(SEQ ID NO: 5264)
CSLQNQQ,
(SEQ ID NO: 5265)
VLWQNQQ,
(SEQ ID NO: 5266)
SKWLLQQ,
(SEQ ID NO: 5267)
SLWSNQQ,
(SEQ ID NO: 5268)
SKRRLQQ,
(SEQ ID NO: 5269)
SVYLNQQ,
(SEQ ID NO: 5270)
SLWLNQQ,
(SEQ ID NO: 5271)
SKAQRKL,
(SEQ ID NO: 5272)
SKALRRQ,
(SEQ ID NO: 5273)
SKAQRLR,
(SEQ ID NO: 5274)
SKAQNQQ,
(SEQ ID NO: 5275)
SKAQRRL,
(SEQ ID NO: 5276)
SKARRQQ,
(SEQ ID NO: 5277)
SKARRLQ,
(SEQ ID NO: 5278)
SKSRRQQ,
(SEQ ID NO: 5279)
SKARLRQ,
(SEQ ID NO: 5280)
SKASKRQ,
(SEQ ID NO: 5281)
VRRQNQQ,
(SEQ ID NO: 5282)
SKAQLYR,
(SEQ ID NO: 5283)
SLFRNQQ,
(SEQ ID NO: 5284)
SKAQLTV;

(ii) an amino acid sequence comprising any portion of an amino acid sequence in (i), e.g., any 2, 3, 4, 5, or 6 amino acids, e.g., consecutive amino acids, thereof; (iii) an amino acid sequence comprising one, two, or three but no more than four modifications relative to am of the amino acid sequences in (i); or
(iv) an amino acid sequence comprising one, two, or three but no more than four differnt amino acids, relative to any one of the amino acid sequences in (i). 133. The AAV particle of any one of embodiments 130-132, wherein [A][B] comprises:

(i)
(SEQ ID NO: 5285)
GSGSPHSLLWNQQ,
(SEQ ID NO: 2060)
GSGSPHSKAQYYV,
(SEQ ID NO: 2061)
GSGSPHSKLRRQQ,
(SEQ ID NO: 5286)
GSGSPHSIWQNQQ,
(SEQ ID NO: 2062)
GSGSPHSKAGCGQ,
(SEQ ID NO: 2063)
GSGSPHSRAQNQQ,
(SEQ ID NO: 2064)
GSGSPHSKRLRQQ,
(SEQ ID NO: 2065)
GSGSPHSLRRNQQ,
(SEQ ID NO: 2066)
GSGSPHSRGRNQQ,
(SEQ ID NO: 5287)
GSGSPHSEIVNQQ,
(SEQ ID NO: 2067)
GSGSPHSSRRNQQ,
(SEQ ID NO: 5288)
GSGSPHCLLQNQQ,
(SEQ ID NO: 2068)
GSGSPHSKAFRLQ,
(SEQ ID NO: 5289)
GSGSPHCLAQNQQ,
(SEQ ID NO: 2070)
GSGSPHFLRQNQQ,
(SEQ ID NO: 2071)
GSGSPHSLRENQQ,
(SEQ ID NO: 5290)
GSGSPHSYLRNQQ,
(SEQ ID NO: 5291)
GSGSPHCSLQNQQ,
(SEQ ID NO: 5292)
GSGSPHVL WQNQQ,
(SEQ ID NO: 2072)
GSGSPHSKWLLQQ,
(SEQ ID NO: 5293)
GSGSPHSLWSNQQ,
(SEQ ID NO: 2073)
GSGSPHSKRRLQQ,
(SEQ ID NO: 5294)
GSGSPHSVYLNQQ,
(SEQ ID NO: 5295)
GSGSPHSLWLNQQ,
(SEQ ID NO: 2074)
GSGSPHSKAQRKL,
(SEQ ID NO: 2075)
GSGSPHSKALRRQ,
(SEQ ID NO: 2076)
GSGSPHSKAQRLR,
(SEQ ID NO: 1801)
GSGSPHSKAQNQQ,
(SEQ ID NO: 2077)
GSGSPHSKAQRRL,
(SEQ ID NO: 2078)
GSGSPHSKARRQQ,
(SEQ ID NO: 2079)
GSGSPHSKARRLQ,
(SEQ ID NO: 2080)
GSGSPHSKSRRQQ,
(SEQ ID NO: 2082)
GSGSPHSKARLRQ,
(SEQ ID NO: 2083)
GSGSPHSKASKRQ,
(SEQ ID NO: 2084)
GSGSPHVRRQNQQ,
(SEQ ID NO: 2085)
GSGSPHSKAQLYR,
(SEQ ID NO: 5296)
GSGSPHSLFRNQQ,
(SEQ ID NO: 2086)
GSGSPHSKAQLTV;

(ii) an amino acid sequence comprising any portion of an amino acid sequence in (i), e.g., ani 2, 3, 4, 5, 6, 7, 8, 9, 10, 11, or 12 amino acids, e.g., consecutive amino acids, thereof;
(iii) an amino acid sequence comprising one, two, or three but no more than four modifications relative to any of the amino acid sequences in (i): or
(iv) an amino acid sequence comprising one, two, or three but no more than four different amino acids, relative to any one of the amino acid sequences in (i)
   134. The AAV particle of any one of embodiments 130-133, wherein the AAV capsid variant further comprises one, two, or all of an amino acid other than T at position 450 (e.g., S, Y, or G), an amino acid other than I at position 451 (e.g., M or L), and/or an amino acid other than N at position 452 (e.g., S), numbered according to SEQ ID NO: 138.
   135. The AAV particle of any one of embodiments 130-134, wherein the AAV capsid variant further comprises an S at position 450 and an M at position 451, numbered according to SEQ ID NO: 138.
   136. The AAV particle of any one of embodiments 130-134, wherein the AAV capsid variant further comprises a Y at position 450, an L at position 451, and an S at position 452, numbered according to SEQ ID NO: 138.
   137. The AAV particle of any one of embodiments 130-134, wherein the AAV capsid variant further comprises a G at position 450, an L at position 451, and an S at position 452, numbered according to SEQ ID NO: 138.
   138. The AAV particle of any one of embodiments 130-137, wherein [A[B] is present in loop IV.
   139. The AAV particle of any one of embodiments 130-138, wherein [A] is present immediately subsequent to position 452, numbered according to SEQ ID NO: 138.
   140. The AAV particle of any one of embodiments 130-139, wherein [A] replaces positions 453-455 (e.g., G453, S454, G455), numbered according to SEQ ID NO: 138.
   141. The AAV particle of any one of embodiments 130-140, wherein [A] is present immediately subsequent to position 452, and wherein [A] replaces positions 453455 (e.g., G453, S454, G455), numbered according to SEQ ID NO: 138.
   142. The AAV particle of any one of embodiments 130-141, wherein [B] is present immediately subsequent to [A].
   143. The AAV particle of any one of embodiments 130-142, wherein [B] replaces positions 456-459 (e.g., Q456, N457, Q458, Q459), numbered according to SEQ ID NO: 138.
   144. The AAV particle of any one of embodiments 130-143, wherein [A][B] replaces positions 453-459 (e.g., G453, S454, G455, Q456, N457, Q458, Q459), numbered according to SEQ ID NO: 138.
   145. The AAV particle of any one of embodiments 130-144, wherein [A][B] is present immediately subsequent to position 452, and wherein [A][B] replaces positions 453-459 (e.g., G453, S454, G455, Q456, N457, Q458, Q459), numbered according to SEQ ID NO: 138.
   146. The AAV particle of any one of embodiments 130-145, wherein the AAV capsid variant comprises, from N-terminus to C-terminus, [A][B].
   147. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises [A][B] (SEQ ID NO: 4699), wherein:
(i) [A] comprises X1 X2 X3 X4 X5 X6, wherein
   (a) X1 is T, M, A, C, 1, R, L, D, F, V, Q, N, or H;
   (b) X2 is I, P, E, N, D, S, A, T, M, or Q;
   (c) X3 is N, E, G, Y, W, M, T, I, K, Q, F, S, V, A, or L;
   (d) X4 is G, D, R, or E;
   (e) X5 is H, Q, N, or D;
   (f) X6 is D or R;
optionally wherein the AAV capsid variant comprises an amino acid modification, e.g., a conservative substitution, of any of the aforesaid amino acids in (a)-(f); and
(ii) [B] comprises SPHKSG (SEQ ID NO: 946).
   148. The AAV particle of embodiment 147, wherein
(a) X1 is: T, M, A, or I;
(b) X2 is: E, I or D;
(c) X3 is: N, Q, Y, I, or V;
(d) X4 is G;
(e) X5 is H; and
(f) X6 is D.
   149. The AAV particle of embodiment 147 or 148, wherein [A] comprises:

(i)
(SEQ ID NO: 5297)
TINGHD,
(SEQ ID NO: 5298)
MPEGHD,
(SEQ ID NO: 5299)
MEGGHD,
(SEQ ID NO: 5300)
MEYGHD,
(SEQ ID NO: 5301)
AEWGHD,
(SEQ ID NO: 5302)
CEWGHD,
(SEQ ID NO: 5303)
ANNGOD,
(SEQ ID NO: 5304)
IPEGHD,
(SEQ ID NO: 5305)
ADMGHD,
(SEQ ID NO: 5306)
IEYGHD,
(SEQ ID NO: 5307)
ADYGHD,
(SEQ ID NO: 5308)
IETGHD,
(SEQ ID NO: 5309)
MEWGHD,
(SEQ ID NO: 5310)
CEYGHD,
(SEQ ID NO: 5311)
RINGHD,
(SEQ ID NO: 5312)
MEIGHD,
(SEQ ID NO: 5313)
LEYGHD,
(SEQ ID NO: 5314)
ADWGHD,
(SEQ ID NO: 5315)
TEIGHD,
(SEQ ID NO: 5316)
TIKDND,
(SEQ ID NO: 5317)
DIMGHD,
(SEQ ID NO: 5318)
FEQGHD,
(SEQ ID NO: 5319)
MEFGHD,
(SEQ ID NO: 5320)
CDQGHD,
(SEQ ID NO: 5321)
LPEGHD,
(SEQ ID NO: 5322)
IENGHD,
(SEQ ID NO: 5323)
MESGHD,
(SEQ ID NO: 5324)
AEIGHD,
(SEQ ID NO: 5325)
VEYGHD,
(SEQ ID NO: 5326)
TSNGDD,
(SEQ ID NO: 5327)
IEVGHD,
(SEQ ID NO: 5328)
MEMGHD,
(SEQ ID NO: 5329)
AEVGHD,
(SEQ ID NO: 5330)
MDAGHD,
(SEQ ID NO: 5331)
VEWGHD,
(SEQ ID NO: 5332)
AEQGHD,
(SEQ ID NO: 5333)
LEWGHD,
(SEQ ID NO: 5334)
MELGHD,
(SEQ ID NO: 5335)
METGHD,
(SEQ ID NO: 5336)
MEAGHD,
(SEQ ID NO: 5337)
TINROR,
(SEQ ID NO: 5338)
IESGHD,
(SEQ ID NO: 5339)
TAKDHD,
(SEQ ID NO: 5340)
MEVGHD,
(SEQ ID NO: 5341)
CEIGHD,
(SEQ ID NO: 5342)
ATNGHD,
(SEQ ID NO: 5343)
MDGGHD,
(SEQ ID NO: 5344)
QEVGHD,
(SEQ ID NO: 5345)
ADQGHD,
(SEQ ID NO: 5346)
NMNGHD,
(SEQ ID NO: 5347)
TPWEHD,
(SEQ ID NO: 5348)
IEMGHD,
(SEQ ID NO: 5349)
TANEHD,
(SEQ ID NO: 5350)
QQQGHD,
(SEQ ID NO: 5351)
TPQDHD,
(SEQ ID NO: 5352)
HDWGHD,
(SEQ ID NO: 5353)
IEGGHD

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   150. The AAV particle of any one of embodiments 147-149, wherein [A][B] comprises:

(i)
(SEQ ID NO: 5354)
TINGHDSPHKR,
(SEQ ID NO: 5355)
MPEGHDSPHKS,
(SEQ ID NO: 5356)
MEGGHDSPHKS,
(SEQ ID NO: 5357)
MEYGHDSPHKS,
(SEQ ID NO: 5358)
AEWGHDSPHKS,
(SEQ ID NO: 5359)
CEWGHDSPHKS,
(SEQ ID NO: 5360)
ANNGODSPHKS,
(SEQ ID NO: 5361)
IPEGHDSPHKS,
(SEQ ID NO: 5362)
ADMGHDSPHKS,
(SEQ ID NO: 5363)
IEYGHDSPHKS,
(SEQ ID NO: 5364)
ADYGHDSPHKS,
(SEQ ID NO: 5365)
IETGHDSPHKS,
(SEQ ID NO: 5366)
MEWGHDSPHKS,
(SEQ ID NO: 5367)
CEYGHDSPHKS,
(SEQ ID NO: 5368)
RINGHDSPHKS,
(SEQ ID NO: 5369)
MEIGHDSPHKS,
(SEQ ID NO: 5370)
LEYGHDSPHKS,
(SEQ ID NO: 5371)
ADWGHDSPHKS,
(SEQ ID NO: 5372)
IEIGHDSPHKS,
(SEQ ID NO: 5373)
TIKDNDSPHKS,
(SEQ ID NO: 5374)
DIMGHDSPHKS,
(SEQ ID NO: 5375)
FEQGHDSPHKS,
(SEQ ID NO: 5376)
MEFGHDSPHKS,
(SEQ ID NO: 5377)
CDQGHDSPHKS,
(SEQ ID NO: 5378)
LPEGHDSPHKS,
(SEQ ID NO: 5379)
IENGHDSPHKS,
(SEQ ID NO: 5380)
MESGHDSPHKS,
(SEQ ID NO: 5381)
AEIGHDSPHKS,
(SEQ ID NO: 5382)
VEYGHDSPHKS,
(SEQ ID NO: 5383)
TSNGDDSPHKS,
(SEQ ID NO: 5384)
IEVGHDSPHKS,
(SEQ ID NO: 5385)
MEMGHDSPHKS,
(SEQ ID NO: 5386)
AEVGHDSPHKS,
(SEQ ID NO: 5387)
MDAGHDSPHKS,
(SEQ ID NO: 5388)
VEWGHDSPHKS,
(SEQ ID NO: 5389)
AEQGHDSPHKS,
(SEQ ID NO: 5390)
LEWGHDSPHKS,
(SEQ ID NO: 5391)
MELGHDSPHKS,
(SEQ ID NO: 5392)
METGHDSPHKS,
(SEQ ID NO: 5393)
MEAGHDSPHKS,
(SEQ ID NO: 5394)
TINRORSPHKS,
(SEQ ID NO: 5395)
IESGHDSPHKS,
(SEQ ID NO: 5396)
TAKDHDSPHKS,
(SEQ ID NO: 5397)
MEVGHDSPHKS,
(SEQ ID NO: 5398)
CEIGHDSPHKS,
(SEQ ID NO: 5399)
ATNGHDSPHKS,
(SEQ ID NO: 5400)
MDGGHDSPHKS,
(SEQ ID NO: 5401)
QEVGHDSPHKS,
(SEQ ID NO: 5402)
ADQGHDSPHKS,
(SEQ ID NO: 5403)
NMNGHDSPHKS,
(SEQ ID NO: 5404)
TPWEHDSPHKS,
(SEQ ID NO: 5405)
IEMGHDSPHKS,
(SEQ ID NO: 5406)
TANEHDSPHKS,
(SEQ ID NO: 5407)
TINGHDSPHKS,
(SEQ ID NO: 5408)
QQQGHDSPHKS,
(SEQ ID NO: 5409)
TPQDHDSPHKS,
(SEQ ID NO: 5410)
HDWGHDSPHKS,
(SEQ ID NO: 5411)
IEGGHDSPHKS

(ii) an amino acid sequence comprising any portion of an amino acid sequence in (i), e.g., any 2, 3, 4, 5, 6, 7, 8, 9, or 10 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   151. The AAV particle of any one of embodiments 147-150, wherein the AAV capsid variant further comprises one, two, three, four, or all of an amino acid other than Q at position 456 (e.g., R or L), N at position 457 (e.g., H, K, or R), Q at position 458 (e.g., R or T), Q at position 459 (H), and/or T at position 460 (N or S), numbered according to SEQ ID NO: 138.
   152. The AAV particle of any one of embodiments 147-151, wherein the AAV capsid variant further comprises an R at position 456, numbered according to SEQ ID NO: 138.
   153. The AAV particle of any one of embodiments 147-151, wherein the AAV capsid variant further comprises an L at position 456, numbered according to SEQ ID NO: 138.
   154. The AAV particle of any one of embodiments 147-153, wherein the AAV capsid variant further comprises an H at position 457 and an R at position 458, numbered according to SEQ ID NO: 138.
   155. The AAV particle of any one of embodiments 147-153, wherein the AAV capsid variant further comprises a K at position 457 and an N at position 460, numbered according to SEQ ID NO: 138.
   156. The AAV particle of any one of embodiments 147-153, wherein the AAV capsid variant further comprises a T at position 458, an H at position 459, and an S at position 460, numbered according to SEQ ID NO: 138.
   157. The AAV particle of any one of embodiments 147-151, wherein the AAV capsid variant further comprises an R at position 456, an R at position 457, and an R at position 458, numbered according to SEQ ID NO: 138.
   158. The AAV particle of any one of embodiments 147-157, wherein [A][B] is present in loop IV.
   159. The AAV particle of any one of embodiments 147-158, wherein [A] is present immediately subsequent to position 449, numbered according to SEQ ID NO: 138.
   160. The AAV particle of any one of embodiments 147-159, wherein [A] replaces positions 450-453 (e.g., T450, 1451, N452, G453), numbered according to SEQ ID NO: 138.
   161. The AAV particle of any one of embodiments 147-160, wherein [A] is present immediately subsequent to position 449, and wherein [A] replaces positions 450-453 (e.g., T450, 1451, N452. G453), numbered according to SEQ ID NO: 138.
   162. The AAV particle of any one of embodiments 147-161, wherein [A][B] replaces positions 450-455 (e.g., T450, 1451, N452, G453, S454, G455), numbered according to SEQ ID NO: 138.
   163. The AAV particle of any one of embodiments 147-162, wherein [A][B] is present immediately subsequent to position 449, and wherein [A][B] replaces positions 450455 (e.g., T450, 1451, N452, G453, S454, G455), numbered according to SEQ ID NO: 138.
   164. The AAV particle of any one of embodiments 147-163, wherein [B] is present immediately subsequent [A], and replaces positions 454 and 455 (e.g., S454 and G455), numbered according to SEQ ID NO: 138.
   165. The AAV particle of any one of embodiments 147-164, wherein [B] is present immediately subsequent to position 455, numbered according to SEQ ID NO: 4, 36, 981, or 982.
   166. The AAV particle of any one of embodiments 147-165, wherein [B] is present immediately subsequent to [A].
   167. The AAV particle of any one of embodiments 147-166, wherein the AAV capsid variant comprises, from N-terminus to C-terminus. [A][B].
   168. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises an amino acid sequence having the following formula: [N1]-[N2]-[N3](SEQ ID NO: 6407), wherein:
(i) [N1] comprises X1, X2, and X3, wherein X2 is S and X3 is G;
(ii) [N2] comprises the amino acid sequence SPH; and
(iii) [N3] comprises X4, X5, and X6, wherein X5 is K.
   169. The AAV particle of embodiment 168, wherein:
(i) X4 of [N3] is S, T, N, or A; and
(ii) X5 of [N3] is A, V, T, S, G, R, L, or N;
optionally wherein the AAV capsid variant comprises an amino acid modification, e.g., a conservative substitution, of any of the aforesaid amino acids in (i) or (ii).
   170. The AAV particle of embodiment 168 or 169, wherein X4 is S and/or X5 is A.
   171. The AAV particle of any one of embodiments 168-170, wherein [N3] comprises SK, TK, NK, AK, KA, KV, KT, KS, KG, KR, KL, or KN.
   172. The AAV particle of any one of embodiments 168-171, wherein [N3] is or comprises SKA, SKV, SKT, SKS, SKG, SKR, TKA, NKA, SKL, SKN, or AKA.
   173. The AAV particle of any one of embodiments 168-172, wherein [N3] is or comprises SKA.
   174. The AAV particle of any one of embodiments 168-173, wherein [N2]-[N3] comprises SPHSK (SEQ ID NO: 4701), SPHTK (SEQ ID NO: 4725), SPHNK (SEQ ID NO: 4726), or SPHAK (SEQ ID NO: 4727).
   175. The AAV particle of any one of embodiments 168-174, wherein [N2]-[N3] is or comprises:

(i)
(SEQ ID NO: 941)
SPHSKA,
(SEQ ID NO: 4737)
SPHSKV,
(SEQ ID NO: 4731)
SPHSKT,
(SEQ ID NO: 962)
SPHSKS,
(SEQ ID NO: 4732)
SPHSKG,
(SEQ ID NO: 978)
SPHSKR,
(SEQ ID NO: 4739)
SPHTKA,
(SEQ ID NO: 4734)
SPHNKA,
(SEQ ID NO: 960)
SPHSKL,
(SEQ ID NO: 4735)
SPHSKN,
or
(SEQ ID NO: 4736)
SPHAKA;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   176. The AAV particle of any one of embodiments 168-175, wherein [N2]-[N3] is or comprises SPHSKA (SEQ ID NO: 941).
   177. The AAV particle of any one of embodiments 168-176, wherein the AAV capsid variant comprises an amino acid other than G at position 453 (e.g., M, T, I, E, S, A, N, V, L, K, H, P, R, W, or D), numbered according to SEQ ID NO: 138 or 981.
   178. The AAV particle of any one of embodiments 168-177, wherein the AAV capsid variant comprises the amino acid G at position 453, numbered according to SEQ ID NO: 138 or 981.
   179. The AAV particle of any one of embodiments 168-178, wherein X1 of [N1] is G, M, T, I, E, S, A, N, V, L, K, H, P, R, W, or D; optionally wherein the AAV capsid variant comprises an amino acid modification, e.g., a conservative substitution, of any of the aforesaid amino acids.
   180. The AAV particle of any one of embodiments 168-179, wherein [N1] comprises SG, GS, MS, TS, IS, ES, SS, AS, NS, VS, LS, KS, HS, PS, RS, WS, or DS.
   181. The AAV particle of any one of embodiments 168-180, wherein IN 1 is or comprises: GSG, MSG, TSG, ISG, ESG, SSG, ASG, NSG, VSG, LSG, KSG, HSG, PSG, RSG, WSG, or DSG.
   182. The AAV particle of any one of embodiments 168-181, wherein [N1] is or comprises GSG.
   183. The AAV particle of any one of embodiments 168-182, wherein [N1]-[N2] comprises SGSPH (SEQ ID NO: 4752).
   184. The AAV particle of any one of embodiments 168-183, wherein [N1]-[N2] is or comprises:

(i)
(SEQ ID NO: 4695)
GSGSPH,
(SEQ ID NO: 4798)
MSGSPH,
(SEQ ID NO: 4800)
TSGSPH,
(SEQ ID NO: 4801)
ISGSPH,
(SEQ ID NO: 4803)
ESGSPH,
(SEQ ID NO: 4804)
SSGSPH,
(SEQ ID NO: 4806)
ASGSPH,
(SEQ ID NO: 4807)
NSGSPH,
(SEQ ID NO: 4786)
VSGSPH,
(SEQ ID NO: 4808)
LSGSPH,
(SEQ ID NO: 4810)
KSGSPH,
(SEQ ID NO: 4811)
HSGSPH,
(SEQ ID NO: 4813)
PSGSPH,
(SEQ ID NO: 4815)
RSGSPH,
(SEQ ID NO: 4817)
WSGSPH,
(SEQ ID NO: 4818)
DSGSPH;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   185. The AAV particle of any one of embodiments 168-184, wherein [N1]-[N2]-[N3] is or comprises:

(i)
(SEQ ID NO: 4697)
GSGSPHSKA,
(SEQ ID NO: 4956)
GSGSPHSKV,
(SEQ ID NO: 4957)
MSGSPHSKA,
(SEQ ID NO: 4959)
TSGSPHSKA,
(SEQ ID NO: 4960)
ISGSPHSKA,
(SEQ ID NO: 4962)
GSGSPHSKT,
(SEQ ID NO: 4963)
ESGSPHSKA,
(SEQ ID NO: 4964)
SSGSPHSKA,
(SEQ ID NO: 4953)
GSGSPHSKS,
(SEQ ID NO: 4966)
ASGSPHSKA,
(SEQ ID NO: 4967)
NSGSPHSKA,
(SEQ ID NO: 4913)
VSGSPHSKA,
(SEQ ID NO: 4968)
LSGSPHSKA,
(SEQ ID NO: 4970)
KSGSPHSKA,
(SEQ ID NO: 4972)
GSGSPHSKG,
(SEQ ID NO: 4945)
GSGSPHSKR,
(SEQ ID NO: 4973)
HSGSPHSKA,
(SEQ ID NO: 4975)
PSGSPHSKA,
(SEQ ID NO: 4977)
RSGSPHSKA,
(SEQ ID NO: 4978)
GSGSPHTKA,
(SEQ ID NO: 4980)
WSGSPHSKA,
(SEQ ID NO: 4981)
DSGSPHSKA,
(SEQ ID NO: 4983)
GSGSPHNKA,
(SEQ ID NO: 4943)
GSGSPHSKL,
(SEQ ID NO: 4994)
GSGSPHSKN,
or
(SEQ ID NO: 4995)
GSGSPHAKA;

(ii) an amino acid sequence comprising any portion of an amino acid sequence in (i), e.g., any 2, 3, 4, 5, 6, 7, 8, or 9 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   186. The AAV particle of any one of embodiments 168-185, wherein [N1]-[N2]-[N3] is or comprises GSGSPHSKA (SEQ ID NO: 4697).
   187. The AAV capsid variant of any one of embodiments 168-186, which comprises an amino acid other than Q at position 456 (e.g., R, P, H, L, K, I, G, S, M, or E), an amino acid other than N at position 457 (e.g., D, V, S, P, T, G, Y, W, E, R, H, K, F, A, I, L, or M), an amino acid other than Q at position 458 (e.g., R, L, A, P, H, T, I, F, K, V, M, G, W, Y, S, E, N, or D), an amino acid other than Q at position 459 (e.g., H, K, A, L, P, E, M, I, S, N, R, Y, C, V, T, W, D, G), and/or an amino acid other than T at position 460 (e.g., I, N, S, H, R, L, D, Y, A, or Q), numbered according to SEQ ID NO: 138.
   188. The AAV particle of any one of embodiments 168-187, wherein the AAV capsid variant comprises an amino acid other than Q at position 462 (e.g., R, P, H, L, K, 1. G, S, M, or E), an amino acid other than N at position 463 (e.g., D, V, S, P, T, G, Y, W, E, R, H, K, F, A, I, L, or M), an amino acid other than Q at position 464 (e.g., R, L, A, P, H, T, I, F, K, V, M, G, W, Y, S, E, N, or D), an amino acid other than Q at position 465 (e.g., H, K, A, L, P, E, M, I, S, N, R, Y, C, V, T, W, D, G), and/or an amino acid other than T at position 466 (e.g., I, N, S, H, R, L, D, Y, A, or Q), numbered according to SEQ ID NO: 981.
   189. The AAV particle of any one of embodiments 168-188, wherein the AAV capsid variant comprises the amino acid Q at position 456, the amino acid N at position 457, the amino acid Q at position 458, the amino acid Q at position 459, and/or the amino acid T at position 460, numbered according to SEQ ID NO: 138.
   190. The AAV particle of any one of embodiments 168-189, wherein the AAV capsid variant comprises the amino acid Q at position 462, the amino acid N at position 463, the amino acid Q at position 464, the amino acid Q at position 465, and/or the amino acid T at position 466, numbered according to SEQ ID NO: 981.
   191. The AAV particle of any one of embodiments 168-190, wherein the AAV capsid variant further comprises [N4] wherein [N4] comprises X7. X8, X9, X10, and X11, wherein:
(a) X7 is Q, R, P, H, L, K, I, G, S, M, or E;
(b) X8 is N, D, V, S, P, T, G, Y, W, E, R, H, K, F, A, I, L, or M;
(c) X9 is Q, R, L, A, P, H, T, I, F, K, V, M, G, W, Y, S, E, N, D;
(d) X10 is Q, H, K, A, L, P, E, M, I, S, N, R, Y, C, V, T, W, D, G; and
(e) X11 is T, I, N, S, H, R, L, D, Y, A, Q;
optionally wherein the AAV capsid variant comprises an amino acid modification, e.g., a conservative substitution, of any of the aforesaid amino acids in (a)-(e).
   192. The AAV particle of embodiment 191, wherein [N4] is or comprises:

(i)
(SEQ ID NO: 5412)
QNQQT,
(SEQ ID NO: 5413)
QNRHT,
(SEQ ID NO: 5414)
RDQQT,
(SEQ ID NO: 5415)
PNLQT,
(SEQ ID NO: 5416)
HVRQT,
(SEQ ID NO: 5417)
PNQHT,
(SEQ ID NO: 5418)
QSQQT,
(SEQ ID NO: 5419)
QNQQI,
(SEQ ID NO: 5420)
QPAKT,
(SEQ ID NO: 5421)
QTQQN,
(SEQ ID NO: 5422)
QNLAT,
(SEQ ID NO: 5423)
QNQLT,
(SEQ ID NO: 5424)
QGQQT,
(SEQ ID NO: 5425)
LNRQS,
(SEQ ID NO: 5426)
HNQQT,
(SEQ ID NO: 5427)
QNPPT,
(SEQ ID NO: 5428)
QNLQT,
(SEQ ID NO: 5429)
QYQQT,
(SEQ ID NO: 5430)
QDQET,
(SEQ ID NO: 5431)
QNHQT,
(SEQ ID NO: 5432)
QDQQT,
(SEQ ID NO: 5433)
HWQQT,
(SEQ ID NO: 5434)
PNQQT,
(SEQ ID NO: 5435)
QNQLI,
(SEQ ID NO: 5436)
PEQQT,
(SEQ ID NO: 5437)
QRTMT,
(SEQ ID NO: 5438)
QNQQH,
(SEQ ID NO: 5439)
LHQHT,
(SEQ ID NO: 5440)
QHRIT,
(SEQ ID NO: 5441)
QYIHT,
(SEQ ID NO: 5442)
QKFET,
(SEQ ID NO: 5443)
QFPST,
(SEQ ID NO: 5444)
HNQQR,
(SEQ ID NO: 5445)
QAIKT,
(SEQ ID NO: 5446)
QNRQT,
(SEQ ID NO: 5447)
QYQHT,
(SEQ ID NO: 5448)
QNPQS,
(SEQ ID NO: 5449)
QHQLT,
(SEQ ID NO: 5450)
QSPPT,
(SEQ ID NO: 5451)
QAKLT,
(SEQ ID NO: 5452)
KSQQT,
(SEQ ID NO: 5453)
QDRPT,
(SEQ ID NO: 5454)
QSQQL,
(SEQ ID NO: 5455)
QAFHT,
(SEQ ID NO: 5456)
QKQQD,
(SEQ ID NO: 5457)
QNAQT,
(SEQ ID NO: 5458)
HNQLT,
(SEQ ID NO: 5459)
QNQQY,
(SEQ ID NO: 5460)
QKLNT,
(SEQ ID NO: 5461)
QNVQT,
(SEQ ID NO: 5462)
QAQQT,
(SEQ ID NO: 5463)
QNLQA,
(SEQ ID NO: 5464)
QTPPT,
(SEQ ID NO: 5465)
QYQHA,
(SEQ ID NO: 5466)
QGQQA,
(SEQ ID NO: 5467)
QPPAT,
(SEQ ID NO: 5468)
QERPT,
(SEQ ID NO: 5469)
QDLQT,
(SEQ ID NO: 5470)
QAMHT,
(SEQ ID NO: 5471)
LNQQT,
(SEQ ID NO: 5472)
QHPST,
(SEQ ID NO: 5473)
PGLQT,
(SEQ ID NO: 5474)
QGIRT,
(SEQ ID NO: 5475)
QAPAT,
(SEQ ID NO: 5476)
QSQQI,
(SEQ ID NO: 5477)
QIPPT,
(SEQ ID NO: 5478)
QTQLT,
(SEQ ID NO: 5479)
QAPST,
(SEQ ID NO: 5480)
QNTYA,
(SEQ ID NO: 5481)
QNQHI,
(SEQ ID NO: 5482)
QNHLT,
(SEQ ID NO: 5483)
QIGMT,
(SEQ ID NO: 5484)
LNKQT,
(SEQ ID NO: 5485)
QLQQT,
(SEQ ID NO: 5486)
QRMST,
(SEQ ID NO: 5487)
QGILT,
(SEQ ID NO: 5488)
QDRQT,
(SEQ ID NO: 5489)
RDWQT,
(SEQ ID NO: 5490)
QNTHD,
(SEQ ID NO: 5491)
PNLQI,
(SEQ ID NO: 5492)
QERST,
(SEQ ID NO: 5493)
QNYQT,
(SEQ ID NO: 5494)
QRTCT,
(SEQ ID NO: 5495)
QIGHT,
(SEQ ID NO: 5496)
QGAIT,
(SEQ ID NO: 5497)
QVPPT,
(SEQ ID NO: 5498)
QVQQI,
(SEQ ID NO: 5499)
LMRQT,
(SEQ ID NO: 5500)
QYSVT,
(SEQ ID NO: 5501)
QAITT,
(SEQ ID NO: 5502)
QKTLT,
(SEQ ID NO: 5503)
QNQWT,
(SEQ ID NO: 5504)
QLHHT,
(SEQ ID NO: 5505)
QNIII,
(SEQ ID NO: 5506)
QGHHT,
(SEQ ID NO: 5507)
QSKVT,
(SEQ ID NO: 5508)
QLPST,
(SEQ ID NO: 5509)
IGKQT,
(SEQ ID NO: 5510)
QAIHT,
(SEQ ID NO: 5511)
QHGLT,
(SEQ ID NO: 5512)
QFMCT,
(SEQ ID NO: 5513)
QHLQT,
(SEQ ID NO: 5514)
QNHQN,
(SEQ ID NO: 5515)
QPART,
(SEQ ID NO: 5516)
QSLQT,
(SEQ ID NO: 5517)
QSQLT,
(SEQ ID NO: 5518)
QDRQS,
(SEQ ID NO: 5519)
QMPST,
(SEQ ID NO: 5520)
QGSLT,
(SEQ ID NO: 5521)
QVPAT,
(SEQ ID NO: 5522)
QDKQT,
(SEQ ID NO: 5523)
HYQQT,
(SEQ ID NO: 5524)
QVPST,
(SEQ ID NO: 5525)
RGEQT,
(SEQ ID NO: 5526)
PGQQT,
(SEQ ID NO: 5527)
QSLQI,
(SEQ ID NO: 5528)
LEQQT,
(SEQ ID NO: 5529)
QNQST,
(SEQ ID NO: 5530)
QKVIT,
(SEQ ID NO: 5531)
QNNDQ,
(SEQ ID NO: 5532)
QSVHT,
(SEQ ID NO: 5533)
QPLGT,
(SEQ ID NO: 5534)
HNQET,
(SEQ ID NO: 5535)
QNLQI,
(SEQ ID NO: 5536)
QIQQT,
(SEQ ID NO: 5537)
QVRNT,
(SEQ ID NO: 5538)
PSNQT,
(SEQ ID NO: 5539)
QVGHT,
(SEQ ID NO: 5540)
QRDIT,
(SEQ ID NO: 5541)
QMPNT,
(SEQ ID NO: 5542)
RGLQT,
(SEQ ID NO: 5543)
QKQQT,
(SEQ ID NO: 5544)
PSLQT,
(SEQ ID NO: 5545)
QRDQT,
(SEQ ID NO: 5546)
QAKGT,
(SEQ ID NO: 5547)
QSAHT,
(SEQ ID NO: 5548)
QSTMT,
(SEQ ID NO: 5549)
QREMT,
(SEQ ID NO: 5550)
QYRAT,
(SEQ ID NO: 5551)
QWQQT,
(SEQ ID NO: 5552)
QRMNT,
(SEQ ID NO: 5553)
GDSQT,
(SEQ ID NO: 5554)
QKIST,
(SEQ ID NO: 5555)
PSMQT,
(SEQ ID NO: 5556)
SPRQT,
(SEQ ID NO: 5557)
MEQQT,
(SEQ ID NO: 5558)
QYQNT,
(SEQ ID NO: 5559)
QHQQT,
(SEQ ID NO: 5560)
INQQT,
(SEQ ID NO: 5561)
PNQQH,
(SEQ ID NO: 5562)
ENROT,
(SEQ ID NO: 5563)
QTQQA,
or
(SEQ ID NO: 5564)
QNQAT;

(ii) an amino acid sequence comprising any portion of an amino acid sequence in (i), e.g., any 2, 3, or 4 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   193. The AAV particle of embodiment 191 or 192, wherein [N1]-[N2]-[N3]-[N4] is or comprises:
(i) the amino acid sequence of any of SEQ ID NOs: 200 or 2887-3076;
(ii) an amino acid sequence comprising any portion of an amino acid sequence in (i), e.g., any 2, 3, 4, 5, 6, 7, 8, 9, 10, 11, 12, or 13 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   194. The AAV particle of any one of embodiments 191-193, wherein [N1]-[N2]-[N3]-[N4] is or comprises GSGSPHSKAQNQQT (SEQ ID NO: 200).
   195. The AAV particle of any one of embodiments 191-193, wherein [N1]-[N2]-[N3]-[N4] is or comprises VSGSPHSKAQNQQT (SEQ ID NO: 903).
   196. The AAV particle of any one of embodiments 168-195, wherein the AAV capsid variant comprises an amino acid other than K at position 449 (e.g., T, E, or N), T at position 450 (e.g., S, E, A, N, V, Q, or G), an amino acid other than I at position 451 (e.g., F, E, V, L, D, S, C, T, A, N, H, R, G, or W), and/or an amino acid other than N at position 452 (e.g., I, P, K, R, H, S, M, Q, D, T, L, A, Y, V, F, E, W, or G), numbered according to SEQ ID NO: 138 or 981.
   197. The AAV particle of any one of embodiments 168-196, wherein the AAV capsid variant comprises the amino acid K at position 449, the amino acid T at position 450, the amino acid I at position 451, and/or the amino acid N at position 452, numbered according to SEQ ID NO: 138 or 981.
   198. The AAV particle of any one of embodiments 168-197, wherein the AAV capsid variant further comprises [N0], wherein [N0] comprises X_{A}, X_{B}, X_{C}, and X_{D}, wherein:
(a) X_{A} is K, T, E, or N;
(b) X_{B} is T, S, E, A, N, V, Q, or G;
(c) X_{C} is I, F, E, V, L, D, S, C, T, A, N, H, R, G, or W; and
(d) X_{D} is N, I, P, K, R, H, S, M, Q, D, T, L, A, Y, V, F, E, W, or G; optionally wherein the AAV capsid variant comprises an amino acid modification, e.g., a conservative substitution, of an of the aforesaid amino acids in (a)-(d).
   199. The AAV particle of embodiment 198, wherein [N0] is or comprises:

(i)
(SEQ ID NO: 5565)
KTII,
(SEQ ID NO: 5566)
KTFP,
(SEQ ID NO: 5567)
KTEK,
(SEQ ID NO: 5568)
KTVN,
(SEQ ID NO: 5569)
KTFN,
(SEQ ID NO: 5570)
KTIN,
(SEQ ID NO: 5571)
TTIN,
(SEQ ID NO: 5572)
KSIN,
(SEQ ID NO: 5573)
KTER,
(SEQ ID NO: 5574)
KELH,
(SEQ ID NO: 5575)
KAIN,
(SEQ ID NO: 5576)
KTDN,
(SEQ ID NO: 5577)
KTFH,
(SEQ ID NO: 5578)
KTSN,
(SEQ ID NO: 5579)
ETIN,
(SEQ ID NO: 5580)
NTIN,
(SEQ ID NO: 5581)
KTEN,
(SEQ ID NO: 5582)
KTSS,
(SEQ ID NO: 5583)
KTCN,
(SEQ ID NO: 5584)
KTEH,
(SEQ ID NO: 5585)
KAEM,
(SEQ ID NO: 5586)
KATN,
(SEQ ID NO: 5587)
KAIK,
(SEQ ID NO: 5588)
KTDK,
(SEQ ID NO: 5589)
KTFK,
(SEQ ID NO: 5590)
KSDQ,
(SEQ ID NO: 5591)
KTEI,
(SEQ ID NO: 5592)
KTID,
(SEQ ID NO: 5593)
KNTN,
(SEQ ID NO: 5594)
KTET,
(SEQ ID NO: 5595)
KTEL,
(SEQ ID NO: 5596)
KNIN,
(SEQ ID NO: 5597)
KTEA,
(SEQ ID NO: 5598)
KTAN,
(SEQ ID NO: 5599)
NTIY,
(SEQ ID NO: 5600)
KTFS,
(SEQ ID NO: 5601)
KTES,
(SEQ ID NO: 5602)
KTTN,
(SEQ ID NO: 5603)
KTED,
(SEQ ID NO: 5604)
KTNN,
(SEQ ID NO: 5605)
KEVH,
(SEQ ID NO: 5606)
KTIS,
(SEQ ID NO: 5607)
KTVR,
(SEQ ID NO: 5608)
KTDR,
(SEQ ID NO: 5609)
ETIK,
(SEQ ID NO: 5610)
KNHI,
(SEQ ID NO: 5611)
KESD,
(SEQ ID NO: 5612)
KTIK,
(SEQ ID NO: 5613)
KTDL,
(SEQ ID NO: 5614)
KTVP,
(SEQ ID NO: 5615)
KTVI,
(SEQ ID NO: 5616)
KAEH,
(SEQ ID NO: 5617)
KNCL,
(SEQ ID NO: 5618)
KTVK,
(SEQ ID NO: 5619)
KNAD,
(SEQ ID NO: 5620)
KTIT,
(SEQ ID NO: 5621)
KNCV,
(SEQ ID NO: 5622)
KNAL,
(SEQ ID NO: 5623)
KVIN,
(SEQ ID NO: 5624)
KTEF,
(SEQ ID NO: 5625)
KTRE,
(SEQ ID NO: 5626)
KOGE,
(SEQ ID NO: 5627)
KSEK,
(SEQ ID NO: 5628)
KNVN,
(SEQ ID NO: 5629)
KGGE,
(SEQ ID NO: 5630)
KEFV,
(SEQ ID NO: 5631)
KSDK,
(SEQ ID NO: 5632)
KTEQ,
(SEQ ID NO: 5633)
KEVQ,
(SEQ ID NO: 5634)
KTEY,
(SEQ ID NO: 5635)
KNCW,
(SEQ ID NO: 5636)
KTDV,
(SEQ ID NO: 5637)
KSDI,
(SEQ ID NO: 5638)
KNSI,
(SEQ ID NO: 5639)
KNSL,
(SEQ ID NO: 5640)
KEVV,
(SEQ ID NO: 5641)
KTEP,
(SEQ ID NO: 5642)
KSEL,
(SEQ ID NO: 5643)
KTWQ,
(SEQ ID NO: 5644)
KTEV,
(SEQ ID NO: 5645)
KAVN,
(SEQ ID NO: 5646)
KGVL,
(SEQ ID NO: 5647)
KTEG,
(SEQ ID NO: 5648)
KTRD,
(SEQ ID NO: 5649)
KTGN,
(SEQ ID NO: 5650)
KNAI,
(SEQ ID NO: 5651)
KAEN,
(SEQ ID NO: 5652)
KAET,
(SEQ ID NO: 5653)
KTVH,
(SEQ ID NO: 5654)
KETA,
(SEQ ID NO: 5655)
KNNL,
(SEQ ID NO: 5656)
EAIN,
(SEQ ID NO: 5657)
KSLN,
(SEQ ID NO: 5658)
KTIP,
or
(SEQ ID NO: 5659)
KTIH;

(ii) an amino acid sequence comprising any portion of an amino acid sequence in (i), e.g., any 2, or 3 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   200. The AAV particle of embodiment 198 or 199, wherein [N0]-[N1]-[N2]-[N3]-[N4] is or comprises:
(i) the amino acid sequence of any one of SEQ ID NOs: 3239-3526 or 3591-3605;
(ii) an amino acid sequence comprising any portion of an amino acid sequence in (i), e.g., any 2, 3, 4, 5, 6, 7, 8, 9, 10, 11, 12, 13, 14, 15, 16, or 17 amino acids, e.g., consecutive amino acids, thereof;
(iii) an amino acid sequence comprising one, two, or three but no more than four modifications to any of the amino acid sequences in (i): or
(iv) an amino acid sequence comprising one, two, or three but no more than four different amino acids, relative to any one of the amino acid sequences in (i).
   201. The AAV particle of any one of embodiments 198-200, wherein [N0]-[N I]-[N2]-[N3]-[N4] is or comprises KTINGSGSPHSKAQNQQT (SEQ ID NO: 5660).
   202. The AAV particle of any one of embodiments 198-200, wherein [N0]-[N1]-[N2]-[N3]-[N4] is or comprises KTERVSGSPHSKAQNQQT (SEQ ID NO: 3589).
   203. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises an amino acid sequence having the following formula: [N1]-[N2]-[N3](SEQ ID NO: 6408), wherein:
(i) [N1] comprises X1, X2, and X3, wherein X2 is an amino acid other than S and X3 is an amino acid other than G;
(ii) [N2] comprises the amino acid sequence SPH; and
(iii) [N3] comprises X4. X5, and X6, wherein X4 is K.
   204. The AAV particle of embodiment 203, wherein:
(i) X5 of [N3] is S, I, T, R, H, Y, L, or M; and
(ii) X6 of [N3] is G, A, L, E, V, R, W, N, Q, or K;
optionally wherein the AAV capsid variant comprises an amino acid modification, e.g., a conservative substitution, of any of the aforesaid amino acids in (i) or (ii).
   205. The AAV particle of embodiment 203 or 204, wherein X5 is S and/or X6 is G.
   206. The AAV particle of any one of embodiments 203-205, wherein [N3] comprises KS, KI, KT, KR, KH, KY, KL, KM, SG, IG, TG, RG, SA, SL, SE, SV, SR, SW, SN, HG, YG, SQ, IV, SK, LW, MG, or MA.
   207. The AAV particle of any one of embodiments 203-206, wherein [N3] is or comprises KSG, KIG, KTG, KRG, KSA, KSL, KSE, KSV, KSR, KSW, KSN, KHG, KYG, KSQ, KIV, KSK, KLW, KMG, or KMA.
   208. The AAV particle of any one of embodiments 203-207, wherein [N3] is or comprises KSG. 209. The AAV particle of any one of embodiments 203-208, wherein [N2]-[N3] comprises SPHKS (SEQ ID NO: 4704), SPHKI (SEQ ID NO: 4713), SPHKT (SEQ ID NO: 4711), SPHKR (SEQ ID NO: 4717), NPHKS (SEQ ID NO: 5661), SPHKH (SEQ ID NO: 4728), SPHKY (SEQ ID NO: 4715). SPHKL (SEQ ID NO: 4714), or SPHKM (SEQ ID NO: 4729).

210. The AAV particle of any one of embodiments 203-209, wherein [N2]-[N3] is or comprises:

(i)
(SEQ ID NO: 946)
SPHKSG,
(SEQ ID NO: 958)
SPHKIG,
(SEQ ID NO: 4738)
SPHKTG,
(SEQ ID NO: 974)
SPHKRG,
(SEQ ID NO: 5662)
NPHKSG,
(SEQ ID NO: 977)
SPHKSA,
(SEQ ID NO: 4740)
SPHKSL,
(SEQ ID NO: 4741)
SPHKSE,
(SEQ ID NO: 4742)
SPHKSV,
(SEQ ID NO: 951)
SPHKSR,
(SEQ ID NO: 4743)
SPHKSW,
(SEQ ID NO: 4744)
SPHKSN,
(SEQ ID NO: 4745)
SPHKHG,
(SEQ ID NO: 966)
SPHKYG,
(SEQ ID NO: 4746)
SPHKSQ,
(SEQ ID NO: 5663)
SPHKIV,
(SEQ ID NO: 4747)
SPHKSK,
(SEQ ID NO: 4748)
SPHKLW,
(SEQ ID NO: 4750)
SPHKMG,
or
(SEQ ID NO: 4751)
SPHKMA;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications relative to any of the amino acid sequences in (i): or
(iv) an amino acid sequence comprising one, two, or three but no more than four different amino acids, relative to any one of the amino acid sequences in (i).
   211. The AAV particle of any one of embodiments 203-210, wherein [N2]-[N3] is or comprises SPHKSG (SEQ ID NO: 946).
   212. The AAV particle of any one of embodiments 203-211, wherein the AAV capsid variant comprises an amino acid other than G at position 453 (e.g., A, K, W, R, L, 1, M, N, T, E, Q, Y, H, F. or V), numbered according to SEQ ID NO: 138 or 981.
   213. The AAV particle of any one of embodiments 203-212, wherein the AAV capsid variant comprises the amino acid G at position 453, numbered according to SEQ ID NO: 138 or 981.
   214. The AAV particle of any one of embodiments 203-214, wherein:
(i) X1 of [N1] is G, A, K, W, R, L, I, M, N, T, E, Q, Y, H, F, or V;
(ii) X2 of [N1] is H, Y, R, Q, N, P, or D;
(iii) X3 of [N1] is D, E, G, V, or N;
optionally wherein the AAV capsid variant comprises an amino acid modification, e.g., a conservative substitution, of any of the aforesaid amino acids in (i), (ii), or (iii).
   215. The AAV particle of any one of embodiments 203-214, wherein X2 of [N1] is H and X3 of [N1] is D.
   216. The AAV particle of any one of embodiments 203-215, wherein X1 of [N1] is G, X2 of [N1] is H and X3 of [N1] is D.
   217. The AAV particle of any one of embodiments 203-216, wherein [N1] comprises GH, HD. GY, GR, GQ, AH, GN, KH, GP, WH, RH, LH, IH, MH, GD, NH, TH, EH, QH, YH, HH, FH, VH, YD, HE, RG, QD, RD, ND, PD, QV, DD, HN, or NG.
   218. The AAV capsid variant of any one of embodiments 203-217, wherein [N1] is or comprises GHD, GYD, GHE. GRG, GQD, GRD, AHD, GND, KHD, GPD, WHD, RHD, LHD, GQV, IHD, MHD, GDD, GHN, NHD, THD. GNG. EHD, QHD, YHD, HHD, FHD, or VHD.
   219. The AAV particle of any one of embodiments 203-218, wherein [N1] is or comprises GHD.
   220. The AAV particle of any one of embodiments 203-219, wherein [N1]-[N2] comprises HDSPH (SEQ ID NO: 4703).
   221. The AAV particle of any one of embodiments 203-220, wherein [N1]-[N2] is or comprises:

(i)
(SEQ ID NO: 4784)
GHDSPH,
(SEQ ID NO: 4829)
GYDSPH,
(SEQ ID NO: 4793)
GHESPH,
(SEQ ID NO: 4788)
GRGSPH,
(SEQ ID NO: 5664)
GHDNPH,
(SEQ ID NO: 4785)
GQDSPH,
(SEQ ID NO: 4831)
GRDSPH,
(SEQ ID NO: 5665)
AHDSPH,
(SEQ ID NO: 4832)
GNDSPH,
(SEQ ID NO: 5666)
KHDSPH,
(SEQ ID NO: 4833)
GPDSPH,
(SEQ ID NO: 5667)
WHDSPH,
(SEQ ID NO: 5668)
RHDSPH,
(SEQ ID NO: 5669)
LHDSPH,
(SEQ ID NO: 4835)
GQVSPH,
(SEQ ID NO: 5670)
IHDSPH,
(SEQ ID NO: 5671)
MHDSPH,
(SEQ ID NO: 4792)
GDDSPH,
(SEQ ID NO: 4836)
GHNSPH,
(SEQ ID NO: 5672)
NHDSPH,
(SEQ ID NO: 5673)
THDSPH,
(SEQ ID NO: 4805)
GNGSPH,
(SEQ ID NO: 5674)
EHDSPH,
(SEQ ID NO: 5675)
QHDSPH,
(SEQ ID NO: 5676)
YHDSPH,
(SEQ ID NO: 5677)
HHDSPH,
(SEQ ID NO: 5678)
FHDSPH,
or
(SEQ ID NO: 5679)
VHDSPH;

(ii) an amino acid sequence comprising any portion of an amino acid sequence in (i), e.g., any 2, 3, 4, or 5 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   222. The AAV particle of any one of embodiments 203-221, wherein [N11-[N2]-[N3] is or comprises:

(i)
(SEQ ID NO: 4698)
GHDSPHKSG,
(SEQ ID NO: 4996)
GHDSPHKIG,
(SEQ ID NO: 4997)
GYDSPHKSG,
(SEQ ID NO: 4998)
GHESPHKSG,
(SEQ ID NO: 4999)
GHDSPHKTG,
(SEQ ID NO: 5000)
GRGSPHKRG,
(SEQ ID NO: 5680)
GHDNPHKSG,
(SEQ ID NO: 4908)
GODSPHKSG,
(SEQ ID NO: 4940)
GHDSPHKSA,
(SEQ ID NO: 5001)
GHDSPHKSL,
(SEQ ID NO: 5003)
GHDSPHKSE,
(SEQ ID NO: 5004)
GRDSPHKSG,
(SEQ ID NO: 5681)
AHDSPHKSG,
(SEQ ID NO: 5005)
GNDSPHKSV,
(SEQ ID NO: 5682)
AHDSPHKIG,
(SEQ ID NO: 4939)
GHESPHKSA,
(SEQ ID NO: 5006)
GODSPHKIG,
(SEQ ID NO: 5007)
GHDSPHKSV,
(SEQ ID NO: 4942)
GHDSPHKSR,
(SEQ ID NO: 5683)
KHDSPHKSG,
(SEQ ID NO: 5008)
GPDSPHKIG,
(SEQ ID NO: 5009)
GPDSPHKSG,
(SEQ ID NO: 5010)
GHDSPHKSW,
(SEQ ID NO: 5684)
WHDSPHKSG,
(SEQ ID NO: 5685)
RHDSPHKSG,
(SEQ ID NO: 5011)
GHDSPHKSN,
(SEQ ID NO: 4937)
GHDSPHKRG,
(SEQ ID NO: 5013)
GHDSPHKHG,
(SEQ ID NO: 5686)
LHDSPHKSG,
(SEQ ID NO: 5014)
GQVSPHKSG,
(SEQ ID NO: 5687)
IHDSPHKSG,
(SEQ ID NO: 5688)
MHDSPHKSG,
(SEQ ID NO: 5015)
GDDSPHKSV,
(SEQ ID NO: 5016)
GHNSPHKSG,
(SEQ ID NO: 5689)
NHDSPHKSG,
(SEQ ID NO: 5690)
THDSPHKSG,
(SEQ ID NO: 5017)
GNGSPHKRG,
(SEQ ID NO: 5691)
EHDSPHKSG,
(SEQ ID NO: 5018)
GHDSPHKYG,
(SEQ ID NO: 5019)
GHDSPHKSQ,
(SEQ ID NO: 5692)
QHDSPHKSG,
(SEQ ID NO: 5693)
RHDSPHKIV,
(SEQ ID NO: 5694)
YHDSPHKSG,
(SEQ ID NO: 5020)
GNDSPHKIG,
(SEQ ID NO: 5695)
HHDSPHKSG,
(SEQ ID NO: 5021)
GHDSPHKSK,
(SEQ ID NO: 5696)
FHDSPHKSG,
(SEQ ID NO: 5022)
GHDSPHKLW,
(SEQ ID NO: 5697)
VHDSPHKSG,
(SEQ ID NO: 5024)
GHDSPHKMG,
(SEQ ID NO: 5025)
GHDSPHKMA,
or
(SEQ ID NO: 4938)
GDDSPHKSG;

(ii) an amino acid sequence comprising any portion of an amino acid sequence in (i), e.g., any 2, 3, 4, 5, 6, 7, 8, or 9 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   223. The AAV particle of any one of embodiments 203-222, wherein [N1]-[N2]-[N3] is or comprises GHDSPHKSG (SEQ ID NO: 4698).
   224. The AAV particle of any one of embodiments 203-223, wherein the AAV capsid variant comprises an amino acid other than Q at position 456 (e.g., R, P, H, K, L, V, A, E, or 1), an amino acid other than N at position 457 (e.g., 1, K, S, H, R, T, D, Y, L, W, F, A, Q, or M), an amino acid other than Q at position 458 (e.g., R, V, K, P, Y, H, L, 1. E, or M), an amino acid other than Q at position 459 (e.g., H, L, E, P, W, D, I, V, S, K, R, C, M, or N), and/or an amino acid other than T at position 460 (e.g., A, E, K, S, I, P, G, or N), numbered according to SEQ ID NO: 138.
   225. The AAV particle of any one of embodiments 203-224, wherein the AAV capsid variant comprises an amino acid other than Q at position 462 (e.g., R, P, H, K, L, V, A, E, or I), an amino acid other than N at position 463 (e.g., I, K, S, H, R, T, D, Y, L, W, F, A, Q, or M), an amino acid other than Q at position 464 (e.g., R, V, K, P, Y, H, L, I, E, or M), an amino acid other than Q at position 465 (e.g., H, L, E, P, W, D, I, V, S, K, R, C, M, or N), and/or an amino acid other than T at position 466 (e.g., A, E, K, S, I, P, G, or N), numbered according to SEQ ID NO: 982.
   226. The AAV particle of any one of embodiments 203-225, wherein the AAV capsid variant comprises the amino acid Q at position 456, the amino acid N at position 457, the amino acid Q at position 458, the amino acid Q at position 459, and/or the amino acid T at position 460, numbered according to SEQ ID NO: 138.
   227. The AAV particle of any one of embodiments 203-226, wherein the AAV capsid variant comprises the amino acid Q at position 462, the amino acid N at position 463, the amino acid Q at position 464, the amino acid Q at position 465, and/or the amino acid T at position 466, numbered according to SEQ ID NO: 138.
   228. The AAV particle of any one of embodiments 203-227, wherein the AAV capsid variant further comprises [N1], wherein [N4] comprises X7, X8, X9, X10, and X11, wherein:
(a) X7 is Q, R, P, H, L, K, 1. G, S, M, or E;
(b) X8 is N, D, V, S, P, T, G, Y, W, E, R, H, K, F, A, I, L, or M;
(c) X9 is Q, R, L, A, P, H, T, 1. F, K, V, M, G, W, Y, S, E, N, D;
(d) X10 is Q, H, K, A, L, P, E, M, I, S, N, R, Y, C, V, T, W, D, G; and
(e) X11 is T, N, S, H, R, L, D, Y, A, Q;
optionally wherein the AAV capsid variant comprises an amino acid modification, e.g., a conservative substitution, of any of the aforesaid amino acids in (a)-(e).
   229. The AAV particle of embodiment 228, wherein [N4] is or comprises:

(i)
(SEQ ID NO: 5412)
QNQQT,
(SEQ ID NO: 5698)
QIRQT,
(SEQ ID NO: 5699)
QNQHA,
(SEQ ID NO: 5543)
QKQQT,
(SEQ ID NO: 5700)
QSVQT,
(SEQ ID NO: 5701)
RSQQT,
(SEQ ID NO: 5702)
QNKLE,
(SEQ ID NO: 5703)
QNQQK,
(SEQ ID NO: 5704)
QHQQA,
(SEQ ID NO: 5705)
QIQHT,
(SEQ ID NO: 5706)
PRQQT,
(SEQ ID NO: 5707)
HTQQT,
(SEQ ID NO: 5708)
QRQHT,
(SEQ ID NO: 5418)
QSQQT,
(SEQ ID NO: 5709)
QNQQS,
(SEQ ID NO: 5710)
RNQET,
(SEQ ID NO: 5478)
QTQLT,
(SEQ ID NO: 5711)
KNQQT,
(SEQ ID NO: 5432)
QDQQT,
(SEQ ID NO: 5426)
HNQQT ,
(SEQ ID NO: 5423)
QNQLT,
(SEQ ID NO: 5712)
QTQQT,
(SEQ ID NO: 5713)
QTQQI,
(SEQ ID NO: 5714)
QSKQA,
(SEQ ID NO: 5715)
QNQPP,
(SEQ ID NO: 5716)
QSPQT,
(SEQ ID NO: 5493)
QNYQT,
(SEQ ID NO: 5431)
QNHQT,
(SEQ ID NO: 5446)
QNRQT,
(SEQ ID NO: 5717)
QNQQG,
(SEQ ID NO: 5482)
QNHLT,
(SEQ ID NO: 5447)
QYQHT,
(SEQ ID NO: 5503)
QNQWT,
(SEQ ID NO: 5718)
QNQHT,
(SEQ ID NO: 5719)
QTRQT,
(SEQ ID NO: 5720)
QNLHT,
(SEQ ID NO: 5471)
LNQQT,
(SEQ ID NO: 5721)
QNQET,
(SEQ ID NO: 5513)
QHLQT,
(SEQ ID NO: 5722)
LNQPT,
(SEQ ID NO: 5723)
QNQDT,
(SEQ ID NO: 5724)
RNQQT,
(SEQ ID NO: 5725)
QNLLT,
(SEQ ID NO: 5726)
QLVIT,
(SEQ ID NO: 5727)
RTQET,
(SEQ ID NO: 5728)
QTHQT,
(SEQ ID NO: 5729)
QNQPA,
(SEQ ID NO: 5730)
QDQHT,
(SEQ ID NO: 5731)
QSQHT,
(SEQ ID NO: 5732)
RNQQI,
(SEQ ID NO: 5733)
VRQQT,
(SEQ ID NO: 5734)
QNQHS,
(SEQ ID NO: 5735)
AWQQT,
(SEQ ID NO: 5736)
QSVPT,
(SEQ ID NO: 5737)
QNIQP,
(SEQ ID NO: 5738)
QNHLN,
(SEQ ID NO: 5739)
LDQQT,
(SEQ ID NO: 5740)
PDQQS,
(SEQ ID NO: 5741)
ESQQT,
(SEQ ID NO: 5742)
QNKQT,
(SEQ ID NO: 5743)
QRQLT,
(SEQ ID NO: 5744)
QUIVT,
(SEQ ID NO: 5745)
QKQST,
(SEQ ID NO: 5746)
QSHQT,
(SEQ ID NO: 5747)
QFVVT,
(SEQ ID NO: 5428)
QNLQT,
(SEQ ID NO: 5419)
QNQQI,
(SEQ ID NO: 5748)
QSQPT,
(SEQ ID NO: 5749)
QNEQT,
(SEQ ID NO: 5516)
QSLQT,
(SEQ ID NO: 5750)
RNRQT,
(SEQ ID NO: 5751)
QSKQT,
(SEQ ID NO: 5752)
QNPLT,
(SEQ ID NO: 5753)
RDQKT,
(SEQ ID NO: 5754)
HNQQN,
(SEQ ID NO: 5755)
QWKRT,
(SEQ ID NO: 5476)
QSQQI,
(SEQ ID NO: 5462)
QAQQT,
(SEQ ID NO: 5756)
QNHQI,
(SEQ ID NO: 5757)
QNQQA,
(SEQ ID NO: 5758)
QNQLN,
(SEQ ID NO: 5759)
QTQPT,
(SEQ ID NO: 5560)
INQQT,
(SEQ ID NO: 5760)
QKQLT,
(SEQ ID NO: 5761)
RNQLA,
(SEQ ID NO: 5762)
RNQQS,
(SEQ ID NO: 5763)
ISIQT,
(SEQ ID NO: 5764)
QNQQN,
(SEQ ID NO: 5765)
QSQQS,
(SEQ ID NO: 5766)
QTVCT,
(SEQ ID NO: 5767)
QYQQI,
(SEQ ID NO: 5768)
QQIMT,
(SEQ ID NO: 5769)
QNEQS,
(SEQ ID NO: 5770)
LNHQT,
(SEQ ID NO: 5771)
QMIHT,
(SEQ ID NO: 5772)
RNHQS,
(SEQ ID NO: 5773)
QKMNT,
(SEQ ID NO: 5774)
QSQQN,
(SEQ ID NO: 5465)
QYQHA;

(ii) an amino acid sequence comprising any portion of an amino acid sequence in (i), e.g., any 2, 3, or 4 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   230. The AAV particle of embodiment 228 or 229, wherein [N1]-[N2]-[N3]-[N4] is or comprises:
(i) the amino acid sequence of any of SEQ ID NOs: 201 or 3160-3237;
(ii) an amino acid sequence comprising any portion of an amino acid sequence in (i), e.g., any 2, 3, 4, 5, 6, 7, 8, 9, 10, 11, 12, or 13 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   231. The AAV particle of any one of embodiments 228-230, wherein [N1]-[N2]-[N3]-[N4] is or comprises GHDSPHKSGQNQQT (SEQ ID NO: 201).
   232. The AAV particle of any one of embodiments 203-231, wherein the AAV capsid variant comprises an amino acid other than K at position 449 (e.g., T), T at position 450 (e.g., A, S, I, V, N, E, Y, C, G, W, or Q), an amino acid other than I at position 451 (e.g., E, V, S, T, N, D, C, G, Q, L, P. A), and/or an amino acid other than N at position 452 (e.g., S, Y, I, K, F, T, D, E, G, V, L, A, M, Q, H. P, or R), numbered according to SEQ ID NO: 138 or 982.
   233. The AAV particle of any one of embodiments 203-232, wherein the AAV capsid variant comprises the amino acid K at position 449, the amino acid T at position 450, the amino acid I at position 451, and/or the amino acid N at position 452, numbered according to SEQ ID NO: 138 or 982.
   234. The AAV particle of any one of embodiments 203-233, wherein the AAV capsid variant further comprises [N0], wherein [N0] comprises X_{A}, X_{B}, X_{C}, and X_{D}, wherein:
(a) XA is K or T,
(b) X_{B} is T, A, S, I, V, N, E, Y, C, G, W, or Q;
(c) X_{C} is 1, E, V, S, T, N, D, C, G, Q, L, P, A; and
(d) X_{D} is N, S, Y, I, K, F, T, D, E, G, V, L, A, M, Q, H, P, or R;
optionally wherein the AAV capsid variant comprises an amino acid modification, e.g., a conservative substitution, of any of the aforesaid amino acids in (a)-(d).
   235. The AAV particle of embodiment 234, wherein [N0] is or comprises:

(i)
(SEQ ID NO: 5575)
KAIN,
(SEQ ID NO: 5570)
KTIN,
(SEQ ID NO: 5601)
KTES,
(SEQ ID NO: 5571)
TTIN,
(SEQ ID NO: 5572)
KSIN,
(SEQ ID NO: 5568)
KTVN,
(SEQ ID NO: 5775)
KSIY,
(SEQ ID NO: 5578)
KTSN,
(SEQ ID NO: 5602)
KTTN,
(SEQ ID NO: 5776)
KIIN,
(SEQ ID NO: 5606)
KTIS,
(SEQ ID NO: 5777)
KAII,
(SEQ ID NO: 5612)
KTIK,
(SEQ ID NO: 5624)
KTEF,
(SEQ ID NO: 5620)
KTIT,
(SEQ ID NO: 5604)
KTNN,
(SEQ ID NO: 5592)
KTID,
(SEQ ID NO: 5778)
KAIS,
(SEQ ID NO: 5779)
KTVD,
(SEQ ID NO: 5780)
KTIE,
(SEQ ID NO: 5647)
KTEG,
(SEQ ID NO: 5623)
KVIN,
(SEQ ID NO: 5645)
KAVN,
(SEQ ID NO: 5781)
KTIY,
(SEQ ID NO: 5576)
KTDN,
(SEQ ID NO: 5583)
KTCN,
(SEQ ID NO: 5782)
KNVV,
(SEQ ID NO: 5595)
KTEL,
(SEQ ID NO: 5783)
KTDA,
(SEQ ID NO: 5644)
KTEV,
(SEQ ID NO: 5642)
KSEL,
(SEQ ID NO: 5784)
KTEM,
(SEQ ID NO: 5632)
KTEQ,
(SEQ ID NO: 5565)
KTII,
(SEQ ID NO: 5785)
KIVN,
(SEQ ID NO: 5567)
KTEK,
(SEQ ID NO: 5581)
KTEN,
(SEQ ID NO: 5786)
KIGN,
(SEQ ID NO: 5787)
KEVM,
(SEQ ID NO: 5788)
KYQV,
(SEQ ID NO: 5597)
KTEA,
(SEQ ID NO: 5586)
KATN,
(SEQ ID NO: 5584)
KTEH,
(SEQ ID NO: 5789)
KTVE,
(SEQ ID NO: 5790)
KAID,
(SEQ ID NO: 5791)
KTIM,
(SEQ ID NO: 5792)
KEVG,
(SEQ ID NO: 5793)
KSEM,
(SEQ ID NO: 5794)
KAQQ,
(SEQ ID NO: 5795)
KCGE,
(SEQ ID NO: 5796)
KASN,
(SEQ ID NO: 5594)
KTET,
(SEQ ID NO: 5797)
KTIG,
(SEQ ID NO: 5798)
KTDP,
(SEQ ID NO: 5799)
KELV
(SEQ ID NO: 5800)
KELM,
(SEQ ID NO: 5801)
KNEI,
(SEQ ID NO: 5802)
KTPN,
(SEQ ID NO: 5803)
KITN,
(SEQ ID NO: 5804)
KTDI,
(SEQ ID NO: 5805)
KTDQ,
(SEQ ID NO: 5806)
KGIN,
(SEQ ID NO: 5807)
KSEI,
(SEQ ID NO: 5627)
KSEK,
(SEQ ID NO: 5808)
KWSA,
(SEQ ID NO: 5809)
KELA,
(SEQ ID NO: 5810)
KQTQ,
(SEQ ID NO: 5811)
KGAD,
(SEQ ID NO: 5812)
KVGE,
(SEQ ID NO: 5813)
KANE,
(SEQ ID NO: 5814)
KTDT,
(SEQ ID NO: 5815)
KTCI,
(SEQ ID NO: 5816)
KELR,
(SEQ ID NO: 5817)
KOQI,
(SEQ ID NO: 5818)
KGVM,
(SEQ ID NO: 5819)
KACD,
(SEQ ID NO: 5820)
KNEL,
(SEQ ID NO: 5821)
KAAE,
(SEQ ID NO: 5822)
KGQN,
(SEQ ID NO: 5823)
KNEF,
(SEQ ID NO: 5824)
KTSI,
(SEQ ID NO: 5616)
KAEH,
(SEQ ID NO: 5825)
KCDQ,
(SEQ ID NO: 5826)
KEIL,
(SEQ ID NO: 5573)
KTER,
(SEQ ID NO: 5650)
KNAI,
(SEQ ID NO: 5588)
KTDK,
(SEQ ID NO: 5827)
KTPD
(SEQ ID NO: 5659)
KTIH,
or
(SEQ ID NO: 5591)
KTEI;

(ii) an amino acid sequence comprising any portion of an amino acid sequence in (i), e.g., any 2, or 3 amino acids, e.g., consecutive amino acids, thereof;
(iii) an amino acid sequence comprising one, two, or three but no more than four modifications relative to any of the amino acid sequences in (i): or
(iv) an amino acid sequence comprising one, two, or three but no more than four different amino acids, relative to any one of the amino acid sequences in (i).
   236. The AAV particle of embodiment 234 or 235, wherein [N0]-[N1]-[N2]-[N3]-[N4] is or comprises:
(i) the amino acid sequence of any one of SEQ ID NOs: 3606-3836;
(ii) an amino acid sequence comprising any portion of an amino acid sequence in (i), e.g., any 2, 3, 4, 5, 6, 7, 8, 9, 10, 11, 12, 13, 14, 15, 16, or 17 amino acids, e.g., consecutive amino acids, thereof.
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).
   237. The AAV particle of any one of embodiments 234-236, wherein [N0]-[N]J-[N2]-[N3]-[N4] is or comprises KTINGHDSPHKSGQNQQT (SEQ ID NO: 5828).
   238. The AAV particle of any one of embodiments 234-236, wherein [N0]-[N I]-[N2]-[N3]-[N4] is or comprises KAEIGHDSPHKSGQNQQT (SEQ ID NO: 1754).
   239. The AAV particle of any one of embodiments 234-236, wherein [N0]-[N1]-[N2]-[N3]-[N4] is or comprises KTEKMSGSPHSKAQNQQT (SEQ ID NO: 3241).
   240. The AAV particle of any one of embodiments 168-239, wherein [N1]-[N2]-[N3] is present in loop IV, e.g., numbered according to SEQ ID NO: 4, 36, 138, 981, or 982.
   241. The AAV particle of any one of embodiments 198-202 or 234-240, wherein [N0] and [N4] are present in loop IV, e.g., numbered according to SEQ ID NO: 4, 36, 138, 981, or 982.
   242. The AAV particle of any one of embodiments 198-202 or 234-241, wherein [N0] is present immediately subsequent to position 448, numbered according to the amino acid sequence of SEQ ID NO: 4, 36, 138, 981, or 982.
   243. The AAV particle of any one of embodiments 198-202 or 234-242, wherein [N0] replaces positions 449-452 (e.g., K449, T450, 1451, and N452), numbered according to SEQ ID NO: 4, 36. 138, 981, or 982.
   244. The AAV particle of any one of embodiments 198-202 or 234-243, wherein [NOI is present immediately subsequent to position 448 and wherein [N0] replaces positions 449-452 (e.g., K449. T450, 1451, and N452), numbered according to SEQ ID NO: 4, 36, 138, 981, or 982.
   245. The AAV particle of any one of embodiments 198-202 or 234-244, wherein [N0] corresponds to positions 449-452 (e.g., K449, T450, 1451, and N452) of any one of SEQ ID NOs: 4, 36, 138, 981, or 982.
   246. The AAV particle of any one of embodiments 168-245, wherein [N1] is present immediately subsequent to position 452, numbered according to SEQ ID NO: 4, 36, 138, 981, or 982.
   247. The AAV particle of any one of embodiments 168-246, wherein [N1] replaces positions 453-455 (e.g., G453, S454, and G455), numbered according to SEQ ID NO: 4, 36, 138, 981, or 982.
   248. The AAV particle of any one of embodiments 168-246, wherein [N1] replaces position 453 (e.g., G453), numbered according to SEQ ID NO: 4, 36, 138, 981, or 982.
   249. The AAV particle of any one of embodiments 168-177, 179-181, 183-185, 187-193, 195-200. 202, or 240-246, wherein:
(i) X1 of [N1] replaces position 453 (e.g., G453);
(ii) X2 of [N1] corresponds to position 454 (e.g., S454); and
(iii) X3 of [N1] corresponds to position 455 (e.g., G455),
wherein (i), (ii), and (iii) are numbered according to SEQ ID NO: 138 or SEQ ID NO: 981.
   250. The AAV particle of any one of embodiments 168-176, 178-201, or 240-246, wherein:
(i) X1 of [N1] corresponds to position 453 (e.g., G453);
(ii) X2 of [N1] corresponds to position 454 (e.g., S454); and
(iii) X3 of [N1] corresponds to position 455 (e.g., G455);
wherein (i), (ii), and (iii) are numbered according to SEQ ID NO: 138 or SEQ ID NO: 981.
   251. The AAV particle of am one of embodiments 203-248, wherein:
(i) X1 of [N1] corresponds to position 453 (e.g., G453);
(ii) X2 of [N1] replaces position 454 (e.g., S454); and
(iii) X3 of [N1] replaces position 455 (e.g., G455),
wherein (i), (ii), and (iii) are numbered according to SEQ ID NO: 138 or SEQ ID NO: 982.
   252. The AAV particle of any one of embodiments 203-248 or 251, wherein [N1] corresponds to positions 453-455 (e.g., G453, H454, D455) of SEQ ID NO 982.
   253. The AAV particle of any one of embodiments 168-176, 178-201, 240-247, or 250, wherein [N1] corresponds to positions 453-455 (e.g., G453, 5454, G455) of SEQ ID NO: 138 or 981.
   254. The AAV particle of any one of embodiments 168-253, wherein [N2] is present immediately subsequent to position 455, numbered according to of SEQ ID NO: 4, 36, 138, 981, or 982.
   255. The AAV particle of any one of embodiments 168-254, wherein [N2] corresponds to positions 456-458 (e.g., S456, P457, and H458) of SEQ ID NO: 981 or 982.
   256. The AAV particle of any one of embodiments 168-254, wherein [N2] corresponds to positions 456-458 (e.g., S456, P457, and H458) of any one of SEQ ID NOs: 4 or 36-59.
   257. The AAV particle of any one of embodiments 168-256, wherein [N2] is present immediately subsequent to [N1].
   258. The AAV particle of any one of embodiments 168-202, 240-247, or 249-257, wherein [N3] corresponds to positions 459-460 (e.g., S459, K460, A461) of SEQ ID NO: 981.
   259. The AAV particle of any one of embodiments 168-202, 240-247, or 249-257, wherein [N3] corresponds to positions 459-460 (e.g., S459, K460, A461) of SEQ ID NO: 36, 38, 39, 40, 41, 42, 43, 44, 45, 46, 47, 48, 49, 50, 51, 52, 53, 54, 55, 57, or 59.
   260. The AAV particle of any one of embodiments 168-259, wherein [N2]-[N3] is present immediately subsequent to position 455, numbered according to any one of SEQ ID NOs: 4, 36, 138. 981, or 982.
   261. The AAV particle of any one of embodiments 168-259, wherein [N2]-[N3] is present immediately subsequent to position 455, numbered according to SEQ ID NO: 981.
   262. The AAV particle of am one of embodiments 168-261, wherein [N2]-[N3] corresponds to positions 456-461 (e.g., S456, P457, H458, S459, K460, A461) of SEQ ID NO: 981.
   263. The AAV particle of any one of embodiments 168-262, wherein [N2]-[N3] corresponds to positions 456-461 (e.g., S456, P457, H458, S459, K460, A461) of any one of SEQ ID NOs: 36, 38, 39, 40, 41, 42, 43, 44, 45, 46, 47, 48, 49, 50, 51, 52, 53, 54, 55, 57, or 59.
   264. The AAV particle of any one of embodiments 203-257 or 259-261, wherein [N3] corresponds to positions 459460 (e.g., K459, 5460, G461) of SEQ ID NO: 982.
   265. The AAV particle of any one of embodiments 203-257 or 259-261, wherein [N3] corresponds to positions 459-460 (e.g., K459, S460, G461) of SEQ ID NO: 37.
   266. The AAV particle of any one of embodiments 203-265, wherein [N2]-[N3] is present immediately subsequent to position 455, numbered according to SEQ ID NO: 982.
   267. The AAV particle of any one of embodiments 203-246, 248, 252, 255, 257, 260, 263-266, wherein [N3] replaces positions 454 and 455 (e.g., S454 and G455), numbered according to SEQ ID NO: 138.
   268. The AAV particle of any one of embodiments 203-246, 248, 252, 255, 257, 260, 263-267, wherein [N3] is present immediately subsequent to [N2] and replaces positions 454 and 455 (e.g., S454 and G455), numbered according to SEQ ID NO: 138.
   269. The AAV particle of any one of embodiments 203-246, 248, 252, 255, 257, 260, 263-268, wherein [N3] is present immediately subsequent to [N1]-[N2] and replaces positions 454 and 455 (e.g., S454 and G455), numbered according to SEQ ID NO: 138.
   270. The AAV particle of any one of embodiments 203-257, 260, 264-269, wherein [N2]-[N3] corresponds to positions 456-461 (e.g., S456, P457, H458, K459, S460, G461) of SEQ ID NO: 982.
   271. The AAV particle of any one of embodiments 203-257, 260, 264-270, wherein [N2]-[N3] corresponds to positions 456-461 (e.g., S456, P457, H458, K459, S460, G461) of SEQ ID NO: 37.
   272. The AAV particle of any one of embodiments 191-202 or 228-271, wherein [N4] is present immediately subsequent to position 455, numbered according to SEQ ID NO: 138.
   273. The AAV particle of any one of embodiments 191-202 or 228-272, wherein [N4] replaces positions 456-46) (e.g., Q456, N457, Q458, Q459, and T460), numbered according to SEQ ID NO: 138.
   274. The AAV particle of any one of embodiments 191-202 or 228-273, wherein [N4] corresponds to positions 462466 (e.g., Q462, N463, Q464, Q465, and T466) of SEQ ID NO: 981 or 982.
   275. The AAV particle of any one of embodiments 191-202 or 228-273, wherein [N4] corresponds to positions 462-466 of any one of SEQ ID NOs: 4 or 36-59.
   276. The AAV particle of any one of embodiments 191-202 or 228-274, wherein [N4] corresponds to positions 456460 (e.g., Q456, N457, Q458, Q459, and T460) of SEQ ID NO: 138.
   277. The AAV particle of any one of embodiments 191-202 or 228-276, wherein [N2]-[N3]-[N4] replaces positions 456460 (e.g., Q456, N457, Q458, Q459, and T460), numbered according to SEQ ID NO: 138.
   278. The AAV particle of any one of embodiments 191-202 or 228-277, wherein [N2]-[N3]-[N4] is present immediately subsequent to position 455, and wherein [N2]-[N3]-[N4] replaces positions 456-460 (e.g., Q456, N457, Q458, Q459, and T460), numbered according to SEQ ID NO: 138.
   279. The AAV particle of any one of embodiments 191-202 or 228-278, wherein [N1]-[N2]-[N3]-[N4] replaces positions 453-460 (e.g., G453, S454, G455, Q456, N457, Q458, Q459, and T460), numbered according to SEQ ID NO: 138.
   280. The AAV particle of any one of embodiments 191-202 or 228-279, wherein [N1]-[N2]-[N3]-[N4] is present immediately subsequent to position 452, and wherein [N1]-[N2]-[N3]-[N4] replaces positions 453-460 (e.g., G453, S454, G455, Q456, N457, Q458, Q459, and T460), numbered according to SEQ ID NO: 138.
   281. The AAV particle of any one of embodiments 191-202, 240-247, 249, 250, 253-263, 266, or 272-280, wherein [N1]-[N2]-[N3]-[N4] corresponds to positions 453466 (e.g., G453, S454, G455, S456, P457, H458, 5459, K460, A461, Q462, N463, Q464, Q465, and T466) of SEQ ID NO: 981.
   282. The AAV particle of any one of embodiments 168-202, 240-247, 249, 250, 253-263, 266, or 272-280, wherein [N1]-[N2]-[N3] corresponds to positions 453461 (e.g., G453, S454, G455, S456, P457, H458, S459, K460, A461) of SEQ ID NO: 981.
   283. The AAV particle of any one of embodiments 228-257, 260, 261, 264-282, wherein [N1]-[N2]-[N3]-[N4] corresponds to positions 453-466 (e.g., G453, H454, D455, S456, P457, H458, K459, S460, G461, Q462, N463, Q464, Q465, T466) of SEQ ID NO: 982.
   284. The AAV particle of any one of embodiments 203-257, 260, 261, 264-283, wherein [N1]-[N2]-[N3] corresponds to positions 453-461 (e.g., G453, H454, D455, S456, P457, H458, K459, S460. G461) of SEQ ID NO: 982.
   285. The AAV particle of any one of embodiments 228-257, 260, 261, 264-282, wherein [N1]-[N2]-[N3]-[N4] corresponds to positions 453466 of any one of SEQ ID NOs: 4 or 36-59.
   286. The AAV particle of any one of embodiments 198-202 or 234-286, wherein [N0]-[N1]-[N2]-[N3]-[N4] replaces positions 449-460 (e.g., K449, T450, 1451, N452, G453, S454, G455, Q456. N457, Q458, Q459, and T460), numbered according to SEQ ID NO: 138.
   287. The AAV particle of any one of embodiments 198-202 or 234-286, wherein [N0]-[N1]-[N2]-[N3]-[N4] is present immediately subsequent to position 448, and wherein [N0]-[N1]-[N2]-[N3]-[N4] replaces positions 449460 (e.g., K449, T450, 1451, N452, G453, S454, G455, Q456, N457, Q458, Q459, and T460), numbered according to SEQ ID NO: 138.
   288. The AAV particle of any one of embodiments 198-202, 240-247, 249, 250, 253-263, 266, 272-281, 286, or 287, wherein [N0]-[N1]-[N2]-[N3]-[N4] corresponds to positions 449-466 (e.g., K449, T450, 1451, N452, G453, 5454, G455, S456, P457, H458, S459, K460, A461, Q462, N463, Q464, Q465, T466) of SEQ ID NO: 981.
   289. The AAV particle of any one of embodiments 234-257, 260, 261, 264-284, 286, or 287, wherein [N0]-[N1]-[N2]-[N3]-[N4] corresponds to positions 449-466 (e.g., K449, T450, 1451, N452, G453, H454, D455, 5456, P457, H458, K459, S460, G461, Q462, N463, Q464, Q465, T466) of SEQ ID NO: 982.
   290. The AAV particle of any one of embodiments 234-257, 260, 261, 264-284, 286, or 287, wherein [N0]-[N1]-[N2]-[N3]-[N4] corresponds to positions 449466 of any one of SEQ ID NOs: 4 or 36-59.
   291. The AAV particle of any one of embodiments 191-202 or 228-290, wherein [N4] is present immediately subsequent to position 461, numbered according to SEQ ID NO: 4, 36, 981, or 982.
   292. The AAV particle of any one of embodiments 191-202 or 228-291, wherein [N4] replaces positions 462-466 (e.g., Q462, N463, Q464, Q465, and T466), numbered according to SEQ ID NO: 4, 36, 981, or 982.
   293. The AAV particle of any one of embodiments 191-202 or 228-292, wherein [N2]-[N3]-[N4] replaces positions 462-466 (e.g., Q462, N463, Q464, Q465, and T466), numbered according to SEQ ID NO: 4, 36, 981, or 982.
   294. The AAV particle of any one of embodiments 191-202 or 228-293, wherein [N2]-[N3]-[N4] is present immediately subsequent to position 455, and wherein [N2]-[N3]-[N4] replaces positions 462-466 (e.g., Q462, N463, Q464, Q465, and T466), numbered according to SEQ ID NO: 4, 36, 981, or 982.
   295. The AAV particle of any one of embodiments 168-294, wherein the AAV capsid variant comprises, from N-terminus to C-terminus, [N2]-[N3].
   296. The AAV particle of any one of embodiments 168-295, wherein the AAV capsid variant comprises, from N-terminus to C-terminus, [N1]-[N2]-[N3].
   297. The AAV particle of any one of embodiments 168-296, wherein the AAV capsid variant comprises, from N-terminus to C-terminus, [N0]-[N1]-[N2]-[N3].
   298. The AAV particle of any one of embodiments 168-297, wherein the AAV capsid variant comprises, from N-terminus to C-terminus, [N1]-[N2]-[N3]-[N4].
   299. The AAV particle of any one of embodiments 168-298, wherein the AAV capsid variant comprises, from N-terminus to C-terminus, [N0]-[N1]-[N2]-[N3]-[N4].
   300. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises the formula [A]-[B] (SEQ ID NO: 4696), wherein:
(i) [A] comprises GSGSPH (SEQ ID NO: 4695); and
(ii) [BJ comprises X1 X2, X3, X4, and X5, wherein;
(a) X1 is S, I, F, V, C, Y, W, R, P, L, Q, M, K, or G;
(b) X2 is K, M, R, F, V, C, P, Y, L, W, G, N, S, T, I, or A;
(c) X3 is A, Y, L, R, W, C, T, F, H, I, P, M, K, S, V, G, Q, or N;
(d) X4 is Q, M, F, K, H, R, C, W, P, V, L, G, S, Y, I, A, T, D, N, or E; and
(e) X5 is A, N, Y, R, K, L, I, M, Q, S, C, W, F, T, G, V, or P;
optionally wherein the AAV capsid variant comprises an amino acid modification, e.g., a conservative substitution, of any of the aforesaid amino acids in (a)-(e).
   301. The AAV particle of embodiment 300, wherein:
(a) X1 is S, L, R, V, or P;
(b) X2 is K, C, F, L, P, R, S, or V;
(c) X3 is A, C, F, I, K, L, M, P, R, T, W, or Y;
(d) X4 is Q, R, S, T, C, F, K, L, P or Y; and
(e) X5 is N, R, S, T, K, M, Q or Y;
optionally wherein the AAV capsid variant comprises an amino acid modification, e.g., a conservative substitution, of any of the aforesaid amino acids in (a)-(e).
   302. The AAV particle of embodiment 300 or 301, wherein [B] comprises SKA, SMY, SKL, SKR, SKW, SRC, SFT, SKF, IVW, SKY, SCH, FPW, SKI, VYY, SLY, SKP, SRF, SRM, SVK, SWA, SLW, SFR, SKK, SYA, SCS, SGA, SFP, SFF, SMC, SKT, SGK, FYR, CRV, YGI, VNC, SLA, WSY, RWL, PSC, SSW, SKG, VPW. SGC, SIT, PKR, SKC, WVP, SFW, RIK, SKM, LRW, LPT, SYM, LLC, RCC, LCV, SYL, QGC, MAF, SFQ, SLC, RPW, RPR, SCP, SVR, SLP, VYH, SYT, LVY, YRY, SWL, CPA, SPP, RWT, PRK, PFV, SKS, WVA, SKV, CAL, SSC, SKN, LCT, STC, SKQ, KSG, SYY, SLT, SCQ, FPF, SVF, GRY, AQA, AQN, YMN, AFY, LKR, RHR, AQK, WRL, CRN, TCN, FFI, AQY, WQN, YFM, ARQ, HQN, IRR, YQN, YWN, AFS, FWN, AQC, MRN, KKN, APN, WKN, ARW, RPN. KVF, AFN, ACS, RLW, SRN, CPN, ACN, FRQ, PFN. FGN, CQN, LFW, TRK, KRN, RQN, VQN, IQN, AQR, PFR, AWN, RSY, LQN, WLN, RRA, AQT, GCT, RYT, TPN, ARM, CFL, PQN, WSN, FKN, KQN, APR, RYN, MIC, TQN, WKS, AAR, LTR, IRG, LVN, FQN, ACQ, WGL, ILR, QIN, ACI, ALR, AHA, CLN, AFV, AQF, RCN, MPC, KTS, PYN, AQS, TRN, LKN, AQM, CTN, PDN. RNY, ACR, CSV, ARI, LPK, SEQ, VRM, NSR, RKR, ARN, QRP, RVV, GQN, YSN, QSN, AKG, CTS, FEN, AKK, KAQ, MYM, KAF, KLK, KRH, KWR, RCR, FTC, KFF, VWQ, KYF, KAR, CHQ, PWQ, KIR, YYQ, LYW, KPK, RFW, RMR, VKK, WAP, LWK, FRP, KKV, YAF, KAC, KRL, CSR, RCP, GAC, KFR, FFG, MCQ, KLF, KTR, GKR, YRQ, RVQ, GIQ, NCQ, KPF, LAW, KRS, SYQ, WLQ, KRR, KGC, KRY, GCQ, FTP, TTC, KRQ, KCF, VPQ, FWS, KFK, IKQ, KAP, FRY, KMI, RWQ, PTQ, KWK, YMR, KAA, LCQ, CCQ, CVQ, KLT, KLC, YLV, AFQ, KWG, KIL, FQI, KAL, KAH, LCL, PRQ, CPQ, VRY, VRC, KMP, KKT, LPY, YHQ, YTR, VYQ, RYQ, WLK, PAQ, MCT, PPD, WTQ, RKQ, KCS, FVQ, KLP, KSE, VAQ, LYQ, KVR, ALQ, SCT, KNS, KRK, CTQ, TCL, YAR, KQR, KRV, SGQ, YYS, LTC, CQS, KAK, KPQ, PFQ, KCT, or VFE.
   303. The AAV particle of any one of embodiments 300-302, wherein [B] comprises SKAQ (SEQ ID NO: 5829), SMYM (SEQ ID NO: 5830), SKAF (SEQ ID NO: 5831), SKLK (SEQ ID NO: 5832). SKRH (SEQ ID NO: 5833), SKWR (SEQ ID NO: 5834), SRCR (SEQ ID NO: 5835), SFTC (SEQ ID NO: 5836), SKFF (SEQ ID NO: 5837). IVWQ (SEQ ID NO: 5838), SKYF (SEQ ID NO: 5839), SKAR (SEQ ID NO: 5840), SCHQ (SEQ ID NO: 5841), FPWQ (SEQ ID NO: 5842), SKIR (SEQ ID NO: 5843), VYYQ (SEQ ID NO: 5844), SLYW (SEQ ID NO: 5845), SKPK (SEQ ID NO: 5846), SRFW (SEQ ID NO: 5847), SRMR (SEQ ID NO: 5848), SVKK (SEQ ID NO: 5849), SWAP (SEQ ID NO: 5850), SLWK (SEQ ID NO: 5851), SFRP (SEQ ID NO: 5852), SKKV (SEQ ID NO: 5853), SYAF (SEQ ID NO: 5854), SKAC (SEQ ID NO: 5855), SKRL (SEQ ID NO: 5856), SCSR (SEQ ID NO: 5857), SRCP (SEQ ID NO: 5858), SGAC (SEQ ID NO: 5859), SKFR (SEQ ID NO: 5860), SFPF (SEQ ID NO: 5861), SFFG (SEQ ID NO: 5862), SMCQ (SEQ ID NO: 5863), SKLF (SEQ ID NO: 5864), SKTR (SEQ ID NO: 5865), SGKR (SEQ ID NO: 5866), FYRQ (SEQ ID NO: 5867), CRVQ (SEQ ID NO: 5868). YGIQ (SEQ ID NO: 5869), VNCQ (SEQ ID NO: 5870), SKPF (SEQ ID NO: 5871), SLAW (SEQ ID NO: 5872), SKRS (SEQ ID NO: 5873). WSYQ (SEQ ID NO: 5874), RWLQ (SEQ ID NO: 5875). PSCQ (SEQ ID NO: 5876), SSWL (SEQ ID NO: 5877), SKRR (SEQ ID NO: 5878), SKGC (SEQ ID NO: 5879). VPWQ (SEQ ID NO: 5880), SKRY (SEQ ID NO: 5881), SGCQ (SEQ ID NO: 5882), SFTP (SEQ ID NO: 5883), STTC (SEQ ID NO: 5884), PKRQ (SEQ ID NO: 5885), SKCF (SEQ ID NO: 5886), WVPQ (SEQ ID NO: 5887), SFWS (SEQ ID NO: 5888). SKFK (SEQ ID NO: 5889), RIKQ (SEQ ID NO: 5890), SKAP (SEQ ID NO: 5891), SFRY (SEQ ID NO: 5892), SKMI (SEQ ID NO: 5893), LRWQ (SEQ ID NO: 5894), LPTQ (SEQ ID NO: 5895), SKWK (SEQ ID NO: 5896), SYMR (SEQ ID NO: 5897), SKAA (SEQ ID NO: 5898), LLCQ (SEQ ID NO: 5899), RCCQ (SEQ ID NO: 5900), LCVQ (SEQ ID NO: 5901), SKLT (SEQ ID NO: 5902), SKLC (SEQ ID NO: 5903), SYLV (SEQ ID NO: 5904), QGCQ (SEQ ID NO: 5905), MAFQ (SEQ ID NO: 5906), SKWG (SEQ ID NO: 5907), SKIL (SEQ ID NO: 5908), SFQI (SEQ ID NO: 5909), SKAL (SEQ ID NO: 5910), SKAH (SEQ ID NO: 5911), SLCL (SEQ ID NO: 5912). RPWQ (SEQ ID NO: 5913), RPRQ (SEQ ID NO: 5914), SCPQ (SEQ ID NO: 5915), SVRY (SEQ ID NO: 5916), SVRC (SEQ ID NO: 5917), SKMP (SEQ ID NO: 5918), SKKT (SEQ ID NO: 5919), SLPY (SEQ ID NO: 5920), VYHQ (SEQ ID NO: 5921), SYTR (SEQ ID NO: 5922), LVYQ (SEQ ID NO: 5923), YRYQ (SEQ ID NO: 5924), SWLK (SEQ ID NO: 5925). CPAQ (SEQ ID NO: 5926), SMCT (SEQ ID NO: 5927), SPPD (SEQ ID NO: 5928), SKRN (SEQ ID NO: 5929), RWTQ (SEQ ID NO: 5930), PRKQ (SEQ ID NO: 5931), SKCS (SEQ ID NO: 5932), PFVQ (SEQ ID NO: 5933), SKLP (SEQ ID NO: 5934), SKSE (SEQ ID NO: 5935). WVAQ (SEQ ID NO: 5936), SLYQ (SEQ ID NO: 5937), SKVR (SEQ ID NO: 5938), CALQ (SEQ ID NO: 5939), SSCT (SEQ ID NO: 5940), SKNS (SEQ ID NO: 5941), SKRK (SEQ ID NO: 5942), LCTQ (SEQ ID NO: 5943), STCL (SEQ ID NO: 5944), SYAR (SEQ ID NO: 5945), SKQR (SEQ ID NO: 5946), SKRV (SEQ ID NO: 5947), KSGQ (SEQ ID NO: 5948), SYYS (SEQ ID NO: 5949), SLTC (SEQ ID NO: 5950), SCQS (SEQ ID NO: 5951). SKAK (SEQ ID NO: 5952), SKPQ (SEQ ID NO: 5953). FPFQ (SEQ ID NO: 5954), SKCT (SEQ ID NO: 5955), SVFE (SEQ ID NO: 5956). GRYQ (SEQ ID NO: 5957). KAQA (SEQ ID NO: 5958), KAQN (SEQ ID NO: 5959), MYMN (SEQ ID NO: 5960), KAFY (SEQ ID NO: 5961). KLKR (SEQ ID NO: 5962), KRHR (SEQ ID NO: 5963), KAQK (SEQ ID NO: 5964). KWRL (SEQ ID NO: 5965), RCRN (SEQ ID NO: 5966), FTCN (SEQ ID NO: 5967), KFFI (SEQ ID NO: 5968), KAQY (SEQ ID NO: 5969), VWQN (SEQ ID NO: 5970), KYFM (SEQ ID NO: 5971), KARQ (SEQ ID NO: 5972), CHQN (SEQ ID NO: 5973), PWQN (SEQ ID NO: 5974), KIRR (SEQ ID NO: 5975), YYQN (SEQ ID NO: 5976). LYWN (SEQ ID NO: 5977), KPKR (SEQ ID NO: 5978), KAFS (SEQ ID NO: 5979). RFWN (SEQ ID NO: 5980), KAQC (SEQ ID NO: 5981), RMRN (SEQ ID NO: 5982), VKKN (SEQ ID NO: 5983), WAPN (SEQ ID NO: 5984), LWKN (SEQ ID NO: 5985), KARW (SEQ ID NO: 5986). FRPN (SEQ ID NO: 5987), KKVF (SEQ ID NO: 5988). YAFN (SEQ ID NO: 5989), KACS (SEQ ID NO: 5990), KRLW (SEQ ID NO: 5991). CSRN (SEQ ID NO: 5992). RCPN (SEQ ID NO: 5993). GACN (SEQ ID NO: 5994). KFRQ (SEQ ID NO: 5995), FPFN (SEQ ID NO: 59%), FFGN (SEQ ID NO: 5997), MCQN (SEQ ID NO: 5998), KLFW (SEQ ID NO: 5999), KTRK (SEQ ID NO: 6000), GKRN (SEQ ID NO: 6001), YRQN (SEQ ID NO: 6002), RVQN (SEQ ID NO: 6003), GIQN (SEQ ID NO: 6004), KAQR (SEQ ID NO: 6005), NCQN (SEQ ID NO: 6006), KPFR (SEQ ID NO: 6007), LAWN (SEQ ID NO: 6008), KRSY (SEQ ID NO: 6009), SYQN (SEQ ID NO: 6010), WLQN (SEQ ID NO: 6011), SCQN (SEQ ID NO: 6012), SWLN (SEQ ID NO: 6013). KRRA (SEQ ID NO: 6014), KAQT (SEQ ID NO: 6015), KGCT (SEQ ID NO: 6016). KRYT (SEQ ID NO: 6017), GCQN (SEQ ID NO: 6018), FTPN (SEQ ID NO: 6019), TCN (SEQ ID NO: 6020), KARM (SEQ ID NO: 6021). KRQN (SEQ ID NO: 6022), KCFL (SEQ ID NO: 6023). VPQN (SEQ ID NO: 6024), FWSN (SEQ ID NO: 6025), KFKN (SEQ ID NO: 6026). IKQN (SEQ ID NO: 6027), KAPR (SEQ ID NO: 6028). FRYN (SEQ ID NO: 6029), KMIC (SEQ ID NO: 6030), RWQN (SEQ ID NO: 6031), PTQN (SEQ ID NO: 6032), KWKS (SEQ ID NO: 6033). YMRN (SEQ ID NO: 6034). KAAR (SEQ ID NO: 6035). LCQN (SEQ ID NO: 6036), CCQN (SEQ ID NO: 6037), CVQN (SEQ ID NO: 6038), KLTR (SEQ ID NO: 6039), KLCT (SEQ ID NO: 6040), KIRG (SEQ ID NO: 6041), YLVN (SEQ ID NO: 6042), AFQN (SEQ ID NO: 6043), KACQ (SEQ ID NO: 6044). KWGL (SEQ ID NO: 6045). KILR (SEQ ID NO: 6046), FQIN (SEQ ID NO: 6047), KACI (SEQ ID NO: 6048). KALR (SEQ ID NO: 6049), KAHA (SEQ ID NO: 6050), LCLN (SEQ ID NO: 6051), KAFV (SEQ ID NO: 6052), PRQN (SEQ ID NO: 6053), CPQN (SEQ ID NO: 6054). KAQF (SEQ ID NO: 6055), VRYN (SEQ ID NO: 6056), VRCN (SEQ ID NO: 6057), KMPC (SEQ ID NO: 6058), KKTS (SEQ ID NO: 6059), LPYN (SEQ ID NO: 6060), YHQN (SEQ ID NO: 6061). KAQS (SEQ ID NO: 6062), YTRN (SEQ ID NO: 6063), VYQN (SEQ ID NO: 6064). RYQN (SEQ ID NO: 6065). WLKN (SEQ ID NO: 6066). KAQM (SEQ ID NO: 6067). PAQN (SEQ ID NO: 6068), MCTN (SEQ ID NO: 6069), PPDN (SEQ ID NO: 6070), KRNY (SEQ ID NO: 6071). WTQN (SEQ ID NO: 6072). KACR (SEQ ID NO: 6073), RKQN (SEQ ID NO: 6074). KCSV (SEQ ID NO: 6075). KART (SEQ ID NO: 6076), FVQN (SEQ ID NO: 6077). KLPK (SEQ ID NO: 6078). KSEQ (SEQ ID NO: 6079). VAQN (SEQ ID NO: 6080), LYQN (SEQ ID NO: 6081), KVRM (SEQ ID NO: 6082), ALQN (SEQ ID NO: 6083), SCTN (SEQ ID NO: 6084). KNSR (SEQ ID NO: 6085). KRKR (SEQ ID NO: 6086). CTQN (SEQ ID NO: 6087), TCLN (SEQ ID NO: 6088), YARN (SEQ ID NO: 6089). KQRP (SEQ ID NO: 6090), KRVV (SEQ ID NO: 6091), SGQN (SEQ ID NO: 6092), YYSN (SEQ ID NO: 6093). LTCN (SEQ ID NO: 6094), CQSN (SEQ ID NO: 6095), KAKG (SEQ ID NO: 6096), KPQN (SEQ ID NO: 6097), PFQN (SEQ ID NO: 6098). KCTS (SEQ ID NO: 6099). VFEN (SEQ ID NO: 6100), or KAKK (SEQ ID NO: 6101).
   304. The AAV particle of any one of embodiments 300-303, wherein [B] is or comprises:

(i)
(SEQ ID NO: 6102)
SKAQA,
(SEQ ID NO: 6103)
SKAQN,
(SEQ ID NO: 6104)
SMYMN,
(SEQ ID NO: 6105)
SKAFY,
(SEQ ID NO: 6106)
SKLKR,
(SEQ ID NO: 6107)
SKRHR,
(SEQ ID NO: 6108)
SKAQK,
(SEQ ID NO: 6109)
SKWRL,
(SEQ ID NO: 6110)
SRCRN,
(SEQ ID NO: 6111)
SFTCN,
(SEQ ID NO: 6112)
SKFFI,
(SEQ ID NO: 6113)
SKAQY,
(SEQ ID NO: 6114)
IVWQN,
(SEQ ID NO: 6115)
SKYFM,
(SEQ ID NO: 6116)
SKARQ,
(SEQ ID NO: 6117)
SCHQN,
(SEQ ID NO: 6118)
FPWQN,
(SEQ ID NO: 6119)
SKIRR,
(SEQ ID NO: 6120)
VYYQN,
(SEQ ID NO: 6121)
SLYWN,
(SEQ ID NO: 6122)
SKPKR,
(SEQ ID NO: 6123)
SKAFS,
(SEQ ID NO: 6124)
SRFWN,
(SEQ ID NO: 6125)
SKAQC,
(SEQ ID NO: 6126)
SRMRN,
(SEQ ID NO: 6127)
SVKKN,
(SEQ ID NO: 6128)
SWAPN,
(SEQ ID NO: 6129)
SLWKN,
(SEQ ID NO: 6130)
SKARW,
(SEQ ID NO: 6131)
SFRPN,
(SEQ ID NO: 6132)
SKKVF,
(SEQ ID NO: 6133)
SYAFN,
(SEQ ID NO: 6134)
SKACS,
(SEQ ID NO: 6135)
SKRLW,
(SEQ ID NO: 6136)
SCSRN,
(SEQ ID NO: 6137)
SRCPN,
(SEQ ID NO: 6138)
SGACN,
(SEQ ID NO: 6139)
SKFRQ,
(SEQ ID NO: 6140)
SFPFN,
(SEQ ID NO: 6141)
SFFGN,
(SEQ ID NO: 6142)
SMCQN,
(SEQ ID NO: 6143)
SKLFW,
(SEQ ID NO: 6144)
SKTRK,
(SEQ ID NO: 6145)
SGKRN,
(SEQ ID NO: 6146)
FYRQN,
(SEQ ID NO: 6147)
CRVQN,
(SEQ ID NO: 6148)
YGIQN,
(SEQ ID NO: 6149)
SKAQR,
(SEQ ID NO: 6150)
VNCQN
(SEQ ID NO: 6151)
SKPFR,
(SEQ ID NO: 6152)
SLAWN,
(SEQ ID NO: 6153)
SKRSY,
(SEQ ID NO: 6154)
WSYQN,
(SEQ ID NO: 6155)
RWLQN,
(SEQ ID NO: 6156)
PSCQN,
(SEQ ID NO: 6157)
SSWLN,
(SEQ ID NO: 6158)
SKRRA,
(SEQ ID NO: 6159)
SKAQT,
(SEQ ID NO: 6160)
SKGCT,
(SEQ ID NO: 6161)
VPWQN,
(SEQ ID NO: 6162)
SKRYT,
(SEQ ID NO: 6163)
SGCQN,
(SEQ ID NO: 6164)
SFTPN,
(SEQ ID NO: 6165)
STTCN,
(SEQ ID NO: 6166)
SKARM,
(SEQ ID NO: 6167)
PKRQN,
(SEQ ID NO: 6168)
SKCFL,
(SEQ ID NO: 6169)
WVPQN,
(SEQ ID NO: 6170)
SFWSN,
(SEQ ID NO: 6171)
SKFKN,
(SEQ ID NO: 6172)
RIKQN,
(SEQ ID NO: 6173)
SKAPR,
(SEQ ID NO: 6174)
SFRYN,
(SEQ ID NO: 6175)
SKMIC,
(SEQ ID NO: 6176)
LRWQN,
(SEQ ID NO: 6177)
LPTQN,
(SEQ ID NO: 6178)
SKWKS,
(SEQ ID NO: 6179)
SYMRN,
(SEQ ID NO: 6180)
SKAAR,
(SEQ ID NO: 6181)
LLCQN,
(SEQ ID NO: 6182)
RCCQN,
(SEQ ID NO: 6183)
LCVQN,
(SEQ ID NO: 6184)
SKLTR,
(SEQ ID NO: 6185)
SKLCT,
(SEQ ID NO: 6186)
SKIRG,
(SEQ ID NO: 6187)
SYLVN,
(SEQ ID NO: 6188)
QGCQN,
(SEQ ID NO: 6189)
MAFQN,
(SEQ ID NO: 6190)
SKACQ,
(SEQ ID NO: 6191)
SKWGL,
(SEQ ID NO: 6192)
SKILR,
(SEQ ID NO: 6193)
SFQIN,
(SEQ ID NO: 6194)
SKACI,
(SEQ ID NO: 6195)
SKALR,
(SEQ ID NO: 6196)
SKAHA,
(SEQ ID NO: 6197)
SLCLN,
(SEQ ID NO: 6198)
SKAFV,
(SEQ ID NO: 6199)
RPWQN,
(SEQ ID NO: 6200)
RPRQN,
(SEQ ID NO: 6201)
SCPQN,
(SEQ ID NO: 6202)
SKAQF,
(SEQ ID NO: 6203)
SVRYN,
(SEQ ID NO: 6204)
SVRCN,
(SEQ ID NO: 6205)
SKMPC,
(SEQ ID NO: 6206)
SKKTS,
(SEQ ID NO: 6207)
SLPYN,
(SEQ ID NO: 6208)
VYHQN,
(SEQ ID NO: 6209)
SKAQS,
(SEQ ID NO: 6210)
SYTRN,
(SEQ ID NO: 6211)
LVYQN,
(SEQ ID NO: 6212)
YRYQN,
(SEQ ID NO: 6213)
SWLKN,
(SEQ ID NO: 6214)
SKAQM,
(SEQ ID NO: 6215)
CPAQN,
(SEQ ID NO: 6216)
SMCTN,
(SEQ ID NO: 6217)
SPPDN,
(SEQ ID NO: 6218)
SKRNY,
(SEQ ID NO: 6219)
RWTQN,
(SEQ ID NO: 6220)
SKACR,
(SEQ ID NO: 6221)
PRKQN,
(SEQ ID NO: 6222)
SKCSV,
(SEQ ID NO: 6223)
SKARI,
(SEQ ID NO: 6224)
PFVQN,
(SEQ ID NO: 6225)
SKLPK,
(SEQ ID NO: 6226)
SKSEQ,
(SEQ ID NO: 6227)
WVAQN,
(SEQ ID NO: 6228)
SLYQN,
(SEQ ID NO: 6229)
SKVRM,
(SEQ ID NO: 6230)
CALQN,
(SEQ ID NO: 6231)
SSCTN,
(SEQ ID NO: 6232)
SKNSR,
(SEQ ID NO: 6233)
SKRKR,
(SEQ ID NO: 6234)
LCTQN,
(SEQ ID NO: 6235)
STCLN,
(SEQ ID NO: 6236)
SYARN,
(SEQ ID NO: 6237)
SKQRP,
(SEQ ID NO: 6238)
SKRVV,
(SEQ ID NO: 6239)
KSGQN,
(SEQ ID NO: 6240)
SYYSN,
(SEQ ID NO: 6241)
SLTCN,
(SEQ ID NO: 6242)
SCQSN,
(SEQ ID NO: 6243)
SKAKG,
(SEQ ID NO: 6244)
SKPQN,
(SEQ ID NO: 6245)
FPFQN,
(SEQ ID NO: 6246)
SKCTS,
(SEQ ID NO: 6247)
SVFEN,
(SEQ ID NO: 6248)
SKAKK,
or
(SEQ ID NO: 6249)
GRYQN;

(ii) an amino acid sequence comprising any portion of an amino acid sequence in (i), e.g., any 2, 3, or 4 amino acids, e.g., consecutive amino acids, thereof;
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i).

305. The AAV particle of any one of embodiments 300-304, wherein [A]-[B] is or comprises:

(i)
(SEQ ID NO: 6250)
GSGSPHSKAQA,
(SEQ ID NO: 6251)
GSGSPHSKAQN,
(SEQ ID NO: 6252)
GSGSPHSMYMN,
(SEQ ID NO: 6253)
GSGSPHSKAFY,
(SEQ ID NO: 6254)
GSGSPHSKLKR,
(SEQ ID NO: 6255)
GSGSPHSKRHR,
(SEQ ID NO: 6256)
GSGSPHSKAQK,
(SEQ ID NO: 6257)
GSGSPHSKWRL,
(SEQ ID NO: 6258)
GSGSPHSRCRN,
(SEQ ID NO: 6259)
GSGSPHSFTCN,
(SEQ ID NO: 6260)
GSGSPHSKFFI,
(SEQ ID NO: 6261)
GSGSPHSKAQY,
(SEQ ID NO: 6262)
GSGSPHIVWQN,
(SEQ ID NO: 6263)
GSGSPHSKYFM,
(SEQ ID NO: 6264)
GSGSPHSKARQ,
(SEQ ID NO: 6265)
GSGSPHSCHQN,
(SEQ ID NO: 6266)
GSGSPHFPWQN,
(SEQ ID NO: 6267)
GSGSPHSKIRR,
(SEQ ID NO: 6268)
GSGSPHVYYQN,
(SEQ ID NO: 6269)
GSGSPHSLYWN,
(SEQ ID NO: 6270)
GSGSPHSKPKR,
(SEQ ID NO: 6271)
GSGSPHSKAFS,
(SEQ ID NO: 6272)
GSGSPHSRFWN,
(SEQ ID NO: 6273)
GSGSPHSKAQC,
(SEQ ID NO: 6274)
GSGSPHSRMRN,
(SEQ ID NO: 6275)
GSGSPHSVKKN,
(SEQ ID NO: 6276)
GSGSPHSWAPN,
(SEQ ID NO: 6277)
GSGSPHSLWKN,
(SEQ ID NO: 6278)
GSGSPHSKARW,
(SEQ ID NO: 6279)
GSGSPHSFRPN,
(SEQ ID NO: 6280)
GSGSPHSKKVF,
(SEQ ID NO: 6281)
GSGSPHSYAFN,
(SEQ ID NO: 6282)
GSGSPHSKACS,
(SEQ ID NO: 6283)
GSGSPHSKRLW,
(SEQ ID NO: 6284)
GSGSPHSCSRN,
(SEQ ID NO: 6285)
GSGSPHSRCPN,
(SEQ ID NO: 6286)
GSGSPHSGACN,
(SEQ ID NO: 6287)
GSGSPHSKFRQ,
(SEQ ID NO: 6288)
GSGSPHSFPFN,
(SEQ ID NO: 6289)
GSGSPHSFFGN,
(SEQ ID NO: 6290)
GSGSPHSMCQN,
(SEQ ID NO: 6291)
GSGSPHSKLFW,
(SEQ ID NO: 6292)
GSGSPHSKTRK,
(SEQ ID NO: 6293)
GSGSPHSGKRN,
(SEQ ID NO: 6294)
GSGSPHFYRQN,
(SEQ ID NO: 6295)
GSGSPHCRVQN,
(SEQ ID NO: 6296)
GSGSPHYGIQN,
(SEQ ID NO: 6297)
GSGSPHSKAQR,
(SEQ ID NO: 6298)
GSGSPHVNCQN,
(SEQ ID NO: 6299)
GSGSPHSKPFR,
(SEQ ID NO: 6300)
GSGSPHSLAWN,
(SEQ ID NO: 6301)
GSGSPHSKRSY,
(SEQ ID NO: 6302)
GSGSPHWSYQN,
(SEQ ID NO: 6303)
GSGSPHRWLON,
(SEQ ID NO: 6304)
GSGSPHPSCQN,
(SEQ ID NO: 6305)
GSGSPHSSWLN,
(SEQ ID NO: 6306)
GSGSPHSKRRA,
(SEQ ID NO: 6307)
GSGSPHSKAQT,
(SEQ ID NO: 6308)
GSGSPHSKGCT,
(SEQ ID NO: 6309)
GSGSPHVPWQN,
(SEQ ID NO: 6310)
GSGSPHSKRYT,
(SEQ ID NO: 6311)
GSGSPHSGCQN,
(SEQ ID NO: 6312)
GSGSPHSFTPN,
(SEQ ID NO: 6313)
GSGSPHSTTCN,
(SEQ ID NO: 6314)
GSGSPHSKARM,
(SEQ ID NO: 6315)
GSGSPHPKRQN,
(SEQ ID NO: 6316)
GSGSPHSKCFL,
(SEQ ID NO: 6317)
GSGSPHWVPQN,
(SEQ ID NO: 6318)
GSGSPHSFWSN,
(SEQ ID NO: 6319)
GSGSPHSKFKN,
(SEQ ID NO: 6320)
GSGSPHRIKQN,
(SEQ ID NO: 6321)
GSGSPHSKAPR,
(SEQ ID NO: 6322)
GSGSPHSFRYN,
(SEQ ID NO: 6323)
GSGSPHSKMIC,
(SEQ ID NO: 6324)
GSGSPHLRWQN,
(SEQ ID NO: 6325)
GSGSPHLPTQN,
(SEQ ID NO: 6326)
GSGSPHSKWKS,
(SEQ ID NO: 6327)
GSGSPHSYMRN,
(SEQ ID NO: 6328)
GSGSPHSKAAR,
(SEQ ID NO: 6329)
GSGSPHLLCQN,
(SEQ ID NO: 6330)
GSGSPHRCCQN,
(SEQ ID NO: 6331)
GSGSPHLCVQN,
(SEQ ID NO: 6332)
GSGSPHSKLTR,
(SEQ ID NO: 6333)
GSGSPHSKLCT,
(SEQ ID NO: 6334)
GSGSPHSKIRG,
(SEQ ID NO: 6335)
GSGSPHSYLVN,
(SEQ ID NO: 6336)
GSGSPHQGCQN,
(SEQ ID NO: 6337)
GSGSPHMAFQN,
(SEQ ID NO: 6338)
GSGSPHSKACQ,
(SEQ ID NO: 6339)
GSGSPHSKWGL,
(SEQ ID NO: 6340)
GSGSPHSKILR,
(SEQ ID NO: 6341)
GSGSPHSFQIN,
(SEQ ID NO: 6342)
GSGSPHSKACI,
(SEQ ID NO: 6343)
GSGSPHSKALR,
(SEQ ID NO: 6344)
GSGSPHSKAHA,
(SEQ ID NO: 6345)
GSGSPHSLCLN,
(SEQ ID NO: 6346)
GSGSPHSKAFV,
(SEQ ID NO: 6347)
GSGSPHRPWQN,
(SEQ ID NO: 6348)
GSGSPHRPRQN,
(SEQ ID NO: 6349)
GSGSPHSCPQN,
(SEQ ID NO: 6350)
GSGSPHSKAQF,
(SEQ ID NO: 6351)
GSGSPHSVRYN,
(SEQ ID NO: 6352)
GSGSPHSVRCN,
(SEQ ID NO: 6353)
GSGSPHSKMPC,
(SEQ ID NO: 6354)
GSGSPHSKKTS,
(SEQ ID NO: 6355)
GSGSPHSLPYN,
(SEQ ID NO: 6356)
GSGSPHVYHQN,
(SEQ ID NO: 6357)
GSGSPHSKAQS,
(SEQ ID NO: 6358)
GSGSPHSYTRN,
(SEQ ID NO: 6359)
GSGSPHLVYQN,
(SEQ ID NO: 6360)
GSGSPHYRYQN,
(SEQ ID NO: 6361)
GSGSPHSWLKN,
(SEQ ID NO: 6362)
GSGSPHSKAQM,
(SEQ ID NO: 6363)
GSGSPHCPAQN,
(SEQ ID NO: 6364)
GSGSPHSMCTN,
(SEQ ID NO: 6365)
GSGSPHSPPDN,
(SEQ ID NO: 6366)
GSGSPHSKRNY,
(SEQ ID NO: 6367)
GSGSPHRWTQN,
(SEQ ID NO: 6368)
GSGSPHSKACR,
(SEQ ID NO: 6369)
GSGSPHPRKQN,
(SEQ ID NO: 6370)
GSGSPHSKCSV,
(SEQ ID NO: 6371)
GSGSPHSKARI,
(SEQ ID NO: 6372)
GSGSPHPFVQN,
(SEQ ID NO: 6373)
GSGSPHSKLPK,
(SEQ ID NO: 6374)
GSGSPHSKSEQ,
(SEQ ID NO: 6375)
GSGSPHWVAQN,
(SEQ ID NO: 6376)
GSGSPHSLYQN,
(SEQ ID NO: 6377)
GSGSPHSKVRM,
(SEQ ID NO: 6378)
GSGSPHCALQN,
(SEQ ID NO: 6379)
GSGSPHSSCTN,
(SEQ ID NO: 6380)
GSGSPHSKNSR,
(SEQ ID NO: 6381)
GSGSPHSKRKR,
(SEQ ID NO: 6382)
GSGSPHLCTQN,
(SEQ ID NO: 6383)
GSGSPHSTCLN,
(SEQ ID NO: 6384)
GSGSPHSYARN,
(SEQ ID NO: 6385)
GSGSPHSKQRP,
(SEQ ID NO: 6386)
GSGSPHSKRVV,
(SEQ ID NO: 6387)
GSGSPHKSGQN,
(SEQ ID NO: 6388)
GSGSPHSYYSN,
(SEQ ID NO: 6389)
GSGSPHSLTCN,
(SEQ ID NO: 6390)
GSGSPHSCQSN,
(SEQ ID NO: 6391)
GSGSPHSKAKG,
(SEQ ID NO: 6392)
GSGSPHSKPQN,
(SEQ ID NO: 6393)
GSGSPHFPFQN,
(SEQ ID NO: 6394)
GSGSPHSKCTS,
(SEQ ID NO: 6395)
GSGSPHSVFEN,
(SEQ ID NO: 6396)
GSGSPHSKAKK,
or
(SEQ ID NO: 6397)
GSGSPHGRYQN;

(ii) an amino acid sequence comprising any portion of an amino acid sequence in (i), e.g., any 2, 3, 4, 5, 6, 7, 8, 9, or 10 amino acids, e.g., consecutive amino acids, thereof.
(iii) an amino acid sequence comprising one, two, or three but no more than four modifications relative to any of the amino acid sequences in (i); or
(iv) an amino acid sequence comprising one, two, or three but no more than four different amino acids, relative to any one of the amino acid sequences in (i)
   306. The AAV particle of any one of embodiments 300-305, wherein [A]-[B] does not comprise the amino acid sequence of GSGSPHSKAQN (SEQ ID NO: 6251).
   307. The AAV particle of any one of embodiments 300-306, wherein the AAV capsid variant comprises one, two, or all of an amino acid other than Q at position 458 (e.g., R, C, S, W, L, F, Y, H, I, V, A, or P), an amino acid other than Q at position 459 (e.g., K, I, R, L or S), and/or an amino acid other than T at position 460 (e.g., R), numbered according to SEQ ID NO: 138.
   308. The AAV particle of any one of embodiments 300-307, wherein the AAV capsid variant comprises:
(i) the amino acid R at position 458;
(ii) the amino acid W at position 458;
(iii) the amino acid Y at position 458;
(iv) the amino acid F at position 458;
(v) the amino acid S at position 458;
(vi) the amino acid C at position 458;
(vii) the amino acid I at position 458;
(viii) the amino acid L at position 458;
(ix) the amino acid P at position 458;
(x) the amino acid I at position 459;
(xi) the amino acid H at position 458: or
(xii) the amino acid V at position 458;
wherein (i)-(xii) are numbered according to SEQ ID NO: 138.
   309. The AAV particle of any one of embodiments 300-307, wherein the AAV capsid variant comprises:
(i) the amino acid R at position 458 and the amino acid K at position 459;
(ii) the amino acid C at position 458 and the amino acid I at position 459;
(iii) the amino acid S at position 458 and the amino acid R at position 459′
(iv) the amino acid L at position 458 and the amino acid K at position 459;
(v) the amino acid F at position 458 and the amino acid K at position 459;
(vi) the amino acid C at position 458 and the amino acid R at position 459;
(vii) the amino acid H at position 458 and the amino acid R at position 459;
(viii) the amino acid I at position 458 and the amino acid L at position 459;
(ix) the amino acid V at position 458 and the amino acid R at position 459;
(x) the amino acid A at position 458 and the amino acid K at position 459;
(xi) the amino acid I at position 458 and the amino acid K at position 459;
(xii) the amino acid C at position 458 and the amino acid S at position 459; or
(xiii) the amino acid C at position 458 and the amino acid L at position 459
wherein (i)-(xiii) are numbered according to SEQ ID NO: 138.
   310. The AAV particle of any one of embodiments 300-307, wherein the AAV capsid variant comprises the amino acid F at position 458, the amino acid K at position 459, and the amino acid R at position 460, numbered according to SEQ ID NO: 138.
   311. The AAV particle of any one of embodiments 300-310, wherein the AAV capsid variant comprises one, two, or all of an amino acid other than T at position 450 (e.g., Y, P, W, R, K, S, or F), an amino acid other than I at position 451 (e.g., R, S, Y, L, V, H, P, A, or F), and/or an amino acid other than N at position 452 (e.g., V, W, A, T, F, Y, L, R, H, S, or M), numbered according to SEQ ID NO: 138.
   312. The AAV particle of any one of embodiments 300-311, wherein the AAV capsid variant comprises the amino acid V at position 452, numbered according to SEQ ID NO: 138.
   313. The AAV particle of any one of embodiments 300-312, wherein the AAV capsid variant comprises the amino acid Y at position 450 and the amino acid V at position 452, numbered according to SEQ ID NO: 138.
   314. The AAV particle of any one of embodiments 300-312, wherein the AAV capsid variant comprises the amino acid R at position 450 and the amino acid Y at position 451, numbered according to SEQ ID NO: 138.
   315. The AAV particle of any one of embodiments 300-311, wherein the AAV capsid variant comprises:
(i) the amino acid P at position 450, the amino acid R at position 451, and the amino acid W at position 452;
(ii) the amino acid Y at position 450, the amino acid S at position 451, and the amino acid A at position 452;
(iii) the amino acid Y at position 450, the amino acid Y at position 451, and the amino acid T at position 452;
(iv) the amino acid P at position 450, the amino acid R at position 451, and the anuno acid F at position 452;
(v) the amino acid W at position 450, the amino acid L at position 451, and the amino acid T at position 452;
(vi) the amino acid R at position 450, the amino acid S at position 451, and the amino acid Y at position 452;
(vii) the amino acid Y at position 450, the amino acid V at position 451, and the amino acid F at position 452;
(viii) the amino acid K at position 450, the amino acid H at position 451, and the amino acid L at position 452;
(ix) the amino acid P at position 450, the amino acid P at position 451, and the amino acid L at position 452;
(x) the amino acid P at position 450, the amino acid A at position 451, and the amino acid R at position 452;
(xi) the amino acid S at position 450, the amino acid R at position 451, and the amino acid R at position 452;
(xii) the amino acid F at position 450, the amino acid F at position 451, and the amino acid H at position 452;
(xiii) the amino acid R at position 450, the amino acid F at position 451, and the amino acid S at position 452;
(xiv) the amino acid Y at position 450, the amino acid S at position 451, and the amino acid M at position 452; or
(xv) the amino acid P at position 450, the amino acid F at position 451, and the amino acid L at position 452;
wherein (i)-(xv) is numbered according to SEQ ID NO: 138.
   316. The AAV particle of any one of embodiments 300-315, wherein the AAV capsid variant comprises:
(i) the amino acid sequence of any one of SEQ ID NOs: 3849-3982, 29844010, 4681-4693;
(ii) an amino acid sequence comprising any portion of an amino acid sequence in (i), e.g., any 2, 3, 4, 5, 6, 7, 8, 9, 10, 11, 12, 13, 14, 15, 16, or 17 amino acids, e.g., consecutive amino acids, thereof;
(iii) an amino acid sequence comprising one, two, or three but no more than four modifications elative to any of the amino acid sequences in (i); or
(iv) an amino acid sequence comprising one, two, or three but no more than four different amino acids, relative to any one of the amino acid sequences in (i).
   317. The AAV particle of any one of embodiments 300-316, wherein the AAV capsid variant does not comprise the amino acid sequence of GSGSPHSKAQNQQ (SEQ ID NO: 1801) or GSGSPHSKAQNQQT (SEQ ID NO: 200).
   318. The AAV particle of any one of embodiments 300-317, wherein [A]-[B] is present in loop TV.
   319. The AAV particle of any one of embodiments 300-318, wherein [A] is present immediately subsequent to position 452, numbered according to SEQ ID NO: 138 or 981.
   320. The AAV particle of any one of embodiments 300-319, wherein [A] replaces positions 453-455 (e.g., G453, S454, G455), numbered according to SEQ ID NO: 138 or 981.
   321. The AAV particle of any one of embodiments 300-320, wherein [A] is present inunediately subsequent to position 452, and wherein [A] replaces positions 453-455 (e.g., G453, S454, G455), numbered according to SEQ ID NO: 138 or 981.
   322. The AAV particle of any one of embodiments 300-321, wherein [B] is present immediately subsequent to [A].
   323. The AAV particle of any one of embodiments 300-322, wherein [B] replaces positions 456 and 457 (e.g., Q456, N457), numbered according to SEQ ID NO: 138.
   324. The AAV particle of am one of embodiments 300-323, wherein [A]-[B] replaces positions 453-457 (e.g., G453, S454, G455, Q456, N457), numbered according to SEQ ID NO: 138.
   325. The AAV particle of any one of embodiments 300-324, wherein [A]-[B] is present immediately subsequent to position 452, and wherein [A]-[B] replaces positions 453-457 (e.g., G453, S454, G455, Q456, N457), numbered according to SEQ ID NO: 138.
   326. The AAV particle of any one of embodiments 300-325, wherein the AAV capsid variant comprises, from N-terminus to C-terminus, [A][B].
   327. The AAV particle of any one of the preceding embodiments, wherein the AAV capsid variant comprises at least one, at least two, at least three, or at least four (e.g., from 1-4 to 1-5) charged amino acid residues (e.g., acidic and/or basic amino acid residues) relative to SEQ ID NO: 138, which is present N-terminal to the amino acid sequence of SPH (e.g., within 1, 2, 3, 4, 5, or 6 amino acids from the start of the SPH amino acid sequence (e.g., within positions 450-455 numbered according to SEQ ID NO: 138)), optionally wherein the amino acid sequence of SPH is present at positions 456-458 numbered according to any one of SEQ ID NOs: 36-59, 981, or 982.
   328. The AAV particle of embodiment 327, wherein the amino acid sequence of SPH is present at positions 456-458 numbered according to any one of SEQ ID NOs: 36-59, 981, or 982.
   329. The AAV particle of embodiment 327 or 328, wherein the AAV capsid variant comprises less than four, less than three, less than two (e.g., two or one) charged amino acid residues (e.g., acidic and/or basic amino acid residues) relative to SEQ ID NO: 138.
   330. The AAV particle of any one of embodiments 327-329, wherein the AAV capsid variant comprises one charged amino acid residues (e.g., an acidic or basic amino acid residue) relative to SEQ ID NO: 138, optionally at any one of positions 450-455 numbered relative to SEQ ID NO: 138.
   331. The AAV particle of any one of embodiments 327-330, wherein the charged amino acid residue is an acidic amino acid (e.g., D or E).
   332. The AAV particle of any one of embodiments 327-331, wherein the charged amino acid residue is a negatively charged amino acid (e.g., D or E).
   333. The AAV particle of any one of embodiments 327-332, wherein the charged amino acid residue is D.
   334. The AAV particle of any one of embodiments 327-333, wherein the charged amino acid residue is E.
   335. The AAV particle of any one of embodiments 327-334, wherein the charged amino acid residue is a basic amino acid (e.g., K, R, or H).
   336. The AAV particle of any one of embodiments 327-335, wherein the charged amino acid residue is a positively charged amino acid (e.g., K, R, or H).
   337. The AAV particle of any one of embodiments 327-336, wherein the charged amino acid residue is H.
   338. The AAV particle of any one of embodiments 327-337, wherein the charged amino acid residue is R.
   339. The AAV particle of any one of embodiments 327-338, wherein the charged amino acid residue is K.
   340. The AAV particle of any one of embodiments 327-339, wherein the AAV capsid variant comprises an acidic amino acid (e.g., E or D) and a basic amino acid (e.g., R, K, or H).
   341. The AAV particle of any one of embodiments 327-340, wherein at least one, two, three or four charged amino acid residues is present within 1, 2, 3, 4, 5, or 6 (e.g., 1-6) amino acids from the start of the SPH amino acid sequence.
   342. The AAV particle of any one of embodiments 327-341, wherein the AAV capsid variant comprises two charged amino acid residues immediately preceding the amino acid sequence of SPH (e.g., at positions 454 and 455, numbered according to SEQ ID NO: 138 or SEQ ID NO: 982).
   343. The AAV particle of any one of embodiments 327-342, wherein the AAV capsid variant comprises a charged amino acid residue (e.g., E) within 1, 2, 3, 4, 5 (e.g., 5) amino acids from the start of the SPH amino acid sequence.
   344. The AAV particle of any one of embodiments 327-343, wherein the AAV capsid variant comprises a charged amino acid residue (e.g., E) at position 451, numbered according to any one of SEQ ID NO: 138, 981, or 982.
   345. The AAV particle of any one of embodiments 327-344, wherein the AAV capsid variant comprises E at position 451, numbered according to any one of SEQ ID NOs: 138, 981, or 982.
   346. The AAV particle of any one of embodiments 327-345, wherein the AAV capsid variant comprises a charged amino acid residue (e.g., R or K) at position 452, numbered according to any one of SEQ ID NOs: 138, 981, or 982.
   347. The AAV particle of any one of embodiments 327-346, wherein the AAV capsid variant comprises R at position 452, numbered according to SEQ ID NO: 138 or SEQ ID NO: 982.
   348. The AAV particle of any one of embodiments 327-347, wherein the AAV capsid variant comprises E at position 451 and R at position 452, numbered according to SEQ ID NO: 138 or SEQ ID NO: 982.
   349. The AAV particle of any one of embodiments 327-348, wherein the AAV capsid variant has decreased tropism for a liver cell or tissue, relative to the tropism of an AAV capsid comprising the amino acid sequence of SEQ ID NO: 138 or SEQ ID NO: 981.
   350. The AAV particle of any one of the preceding embodiments, wherein the AAV capsid variant comprises at least one, at least two, at least three, or at least four (e.g., from 14 to 1-5) charged amino acid residues (e.g., basic amino acid residues) relative to SEQ ID NO: 138, which is present C-terminal to the amino acid sequence of SPH (e.g., within 1, 2, 3, 4, 5, 6, or 7 amino acids from the end of the SPH amino acid sequence (e.g., within positions 459-465 numbered according to any one of SEQ ID NOs: 36-59, or 981)), optionally wherein the amino acid sequence of SPH is present at positions 456-458 numbered according to any one of SEQ ID NOs: 36-59, 981, or 982.
   351. The AAV particle of embodiment 350, wherein the amino acid sequence of SPH is present at positions 456-458 numbered according to any one of SEQ ID NOs: 36-59, 981, or 982.
   352. The AAV particle of embodiment 350 or 351, wherein the AAV capsid variant comprises less than four, less than three, less than two (e.g., two or one) charged amino acid residues (e.g., basic amino acid residues) relative to SEQ ID NO: 138.
   353. The AAV particle of any one of embodiments 350-352, wherein the AAV capsid variant comprises one charged amino acid residues (e.g., a basic amino acid residue) relative to SEQ ID NO: 138, optionally at any one of positions 456460, numbered according to SEQ ID NO: 138, or at positions 462466, numbered according to any one of SEQ ID NOs: 36-59, 981, or 982.
   354. The AAV particle of any one of embodiments 350-353, wherein the charged amino acid residue is a basic amino acid (e.g., R or K).
   355. The AAV particle of any one of embodiments 350-354, wherein the charged amino acid residue is a positively charged amino acid (e.g., R or K).
   356. The AAV particle of any one of embodiments 350-355, wherein the charged amino acid residue is R.
   357. The AAV particle of any one of embodiments 350-355, wherein the charged amino acid residue is K.
   358. The AAV particle of any one of embodiments 350-357, wherein at least one, two, three or four charged amino acid residues is present within 1, 2, 3, 4, 5, 6, 7 (e.g., 1-7) amino acids from the end of the SPH amino acid sequence.
   359. The AAV particle of any one of embodiments 350-358, wherein the AAV capsid variant comprises a charged amino acid residue (e.g., K or R) immediately after the SPH sequence (e.g., at position 459 numbered according to SEQ ID NO: 981).
   360. The AAV particle of any one of embodiments 350-359, wherein the AAV capsid variant comprises a charged amino acid residue (e.g., K or R) at position 459, numbered according to SEQ ID NO: 138 or SEQ ID NO: 982.
   361. The AAV particle of any one of embodiments 350-360, wherein the AAV capsid variant comprises K at position 459, numbered according to SEQ ID NO: 981.
   362. The AAV particle of any one of embodiments 350-360, wherein the AAV capsid variant comprises R at position 459, numbered according to SEQ ID NO: 981.
   363. The AAV particle of any one of embodiments 350-362, wherein the AAV capsid variant comprises a charged amino acid residue (e.g., R or K) at one, two three, four, five, or all of positions 460, 461, 462, 463, 464, and/or 465, numbered according to SEQ ID NO: 138 or 981.
   364. The AAV particle of any one of embodiments 300-326 or 350-363, wherein the AAV capsid variant has increased tropism for a liver cell or tissue, relative to the tropism of an AAV capsid comprising the amino acid sequence of SEQ ID NO: 138.
   365. The AAV particle of any one of embodiments 300-326 or 350-364, wherein the AAV capsid variant is enriched at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 150, at least 160, at least 170, at least 180, at least 190, or at least 200-fold, in the liver compared to an AAV capsid comprising the amino acid sequence of SEQ ID NO: 138, e.g., when measured by an assay as described in Example 4.
   366. The AAV particle of any one of embodiments 300-326, 364, or 365, wherein the AAV capsid variant has reduced tropism for a CNS cell or tissue, e.g., a brain cell, brain tissue, spinal cord cell, or spinal cord tissue, relative to the tropism of an AAV capsid comprising the amino acid sequence of SEQ ID NO: 138.
   367. The AAV particle of any one of embodiments 300-326 or 364-366, wherein the AAV capsid variant shows preferential transduction in a liver region relative to the transduction in the brain and/or dorsal root ganglia (DRG).
   368. The AAV particle of any one of embodiments 300-326 or 364-367, wherein the AAV capsid variant shows preferential transduction in a liver region relative to the transduction in the heart and/or muscle (e.g., quadriceps).
   369. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises:
(a) the amino acid sequence of any of the sequences provided in Table 1, 2A, 2B, or 20-26;
(b) an amino acid sequence comprising at least 3, at least 4, at least 5, at least 6, at least 7, at least 8, at least 9, at least 10, at least 11, at least 12, at least 13, at least 14, at least 15, at least 16, or at least 17 consecutive amino acids from any one of the sequences provided in Table 1, 2A, 2B, or 20-26; or
(c) an amino acid sequence comprising at least one, at least two, or at least three but no more than four different amino acids, relative to any one of the sequences provided in Table 1, 2A, 2B, or 20-26: or
(d) an amino acid sequence comprising at least one, at least two, or at least three but no more than four modifications relative to the amino acid sequence of any one of the sequences provided in Table 1, 2A, 2B, or 20-26.
   370. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises:
(a) the amino acid sequence of any of SEQ ID NOs: 945-980 or 985-986;
(b) an amino acid sequence comprising at least 3, at least 4, or at least 5 consecutive amino acids from any one of SEQ ID NOs: 945-980 or 985-986; or
(c) an amino acid sequence comprising at least one, at least two, or at least three but no more than four different amino acids, relative to the amino acid sequence of any one of SEQ ID NOs: 945-980 or 985-986;
(d) an amino sequence comprising at least one, at least two, or at least three but no more than four modifications relative to the amino acid sequence of any one of SEQ ID NOs: 945-980 or 985-986.
   371. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises:
(a) the amino acid sequence of any of SEQ ID NOs: 2, 200, 201, 941, 943, 204, 208, 404, or 903-909;
(b) an amino acid sequence comprising at least 3, at least 4, at least 5, at least 6, at least 7, at least 8, at least 9, at least 10, at least 11, at least 12, or at least 13 consecutive amino acids from any one of SEQ ID NOs: 2, 200, 201, 941, 943, 204, 208, 404, or 903-909;
(c) an amino acid sequence comprising at least one, at least two, or at least three but no more than four different amino acids, relative to the amino acid sequence of any one of SEQ ID NOs: 2, 200, 201, 941, 943, 204, 208, 404, or 903-909: or
(d) an amino acid sequence comprising at least one, at least two, or at least three but no more than four modifications relative to the amino acid sequence of any one of SEQ ID NOs: 2, 200, 201. 941, 943, 204, 208, 404, or 903-909.
   372. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises:
(a) the amino acid sequence of any of SEQ ID NOs: 3849-4051 or 4681-4693;
(b) an amino acid sequence comprising at least 3, at least 4, at least 5, at least 6, at least 7, at least 8, at least 9, at least 10, at least 11, at least 12, at least 13, at least 14, at least 15, at least 16 or at least 17 consecutive amino acids from any one of SEQ ID NOs: 3849-4051 or 4681-4693;
(c) an amino acid sequence comprising at least one, at least two, or at least three but no more than four different amino acids, relative to the amino acid sequence of any one of SEQ ID NOs: 3849-4051 or 4681-4693: or
(d) an amino acid sequence comprising at least one, at least two, or at least three but no more than four modifications relative to the amino acid sequence of any one of SEQ ID NOs: 3849-4051 or 4681-4693.
   373. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises:
(a) the amino acid sequence of any of SEQ ID NOs: 40524092;
(b) an amino acid sequence comprising at least 3, at least 4, at least 5, at least 6, at least 7, at least 8, at least 9, at least 10, at least 11, at least 12, at least 13, at least 14, at least 15, at least 16 or at least 17 consecutive amino acids from any one of SEQ ID NOs: 4052-4092;
(c) an amino acid sequence comprising at least one, at least two, or at least three but no more than four different amino acids, relative to the amino acid sequence of any one of SEQ ID NOs: 4052-4092; or
(d) an amino acid sequence comprising at least one, at least two, or at least three but no more than four modifications relative to the amino acid sequence of any one of SEQ ID NOs: 4052-4092.
   374. An adeno-associated virus (AAV) particle comprising an AAV capsid variant (e.g., an AAV9 capsid variant) and a viral genome comprising a syntaxin-binding protein 1 (STXBP1)-encoding sequence (e.g., encoding a human STXBP1 protein), wherein the AAV capsid variant comprises:
(a) the amino acid sequence of any of SEQ ID NOs: 4056, 4058, 4059, 4062-4064, 4066, 4067, 4080, 4084, 4090, or 4095-4097;
(b) an amino acid sequence comprising at least 3, at least 4, at least 5, at least 6, at least 7, at least 8, at least 9, at least 10, at least 11, at least 12, at least 13, at least 14, at least 15, at least 16 or at least 17 consecutive amino acids from any one of SEQ ID NOs: 4056, 4058, 4059, 4062-4064, 4066, 4067, 4080, 4084, 4090, or 4095-4097;
(c) an amino acid sequence comprising at least one, at least two, or at least three but no more than four different amino acids, relative to the amino acid sequence of am one of SEQ ID NOs: 4056, 4058, 4059, 4062-4064, 4066, 4067, 4080, 4084, 4090, or 4095-4097, or
(d) an amino acid sequence comprising at least one, at least two, or at least three but no more than four modifications relative to the amino acid sequence of any one of SEQ ID NOs: 4056, 4058, 4059, 4062-4064, 4066, 4067, 4080, 4084, 4090, or 4095-4097.
   375. The AAV particle of embodiment 369 or 371, wherein the AAV capsid variant comprises an amino acid sequence comprising at least 4, at least 5, at least 6, at least 7, at least 8, at least 9, at least 10, at least 11, at least 12, or at least 13 consecutive amino acids from any one of SEQ ID NOs: 2, 200, 201, 941, 943, 204, 208, 404, or 903-909.
   376. The AAV particle of any one of embodiments 369-374, wherein the at least 3 consecutive amino acids comprise SPH.
   377. The AAV particle of any one of embodiments 369-371 or 376, wherein the at least 4 consecutive amino acids comprise SPHS (SEQ ID NO: 4700).
   378. The AAV particle of any one of embodiments 369-371, 376, or 377, wherein the at least 5 consecutive amino acids comprise SPHSK (SEQ ID NO: 4701).
   379. The AAV particle of any one of embodiments 369-371 or 376-378, wherein the at least 6 consecutive amino acids comprise SPHSKA (SEQ ID NO: 941).
   380. The AAV particle of embodiment 369-371, wherein the at least 3 consecutive amino acids comprise HDS.
   381. The AAV particle of any one of embodiments 369-371 or 380, wherein the at least 4 consecutive amino acids comprise HDSP (SEQ ID NO: 4702).
   382. The AAV particle of any one of embodiments 369-371, 380, or 381, wherein the at least 5 consecutive amino acids comprise HDSPH (SEQ ID NO: 4703).
   383. The AAV particle of any one of embodiments 369-371 or 380-382, wherein the at least 6 consecutive amino acids comprise HDSPHK (SEQ ID NO: 2).
   384. The AAV particle of any one of embodiments 369-371, wherein:
(i) the at least 3 consecutive amino acids comprise SPH;
(ii) the at least 4 consecutive amino acids comprise SPHK (SEQ ID NO: 6398);
(iii) the at least 5 consecutive amino acids comprise SPHKY (SEQ ID NO: 4715); and/or
(iv) the at least 6 consecutive amino acids comprise SPHKYG (SEQ ID NO: 966).
   385. The AAV particle of embodiment 369 or 371, wherein the AAV capsid variant comprises an amino acid sequence comprising at least one, at least two, or at least three but no more than four modifications relative to the amino acid sequence of any one of SEQ ID NOs: 2, 200, 201, 941, 943, 204, 208, 404, or 903-909.
   386. The AAV particle of any one of embodiments 369, 371, or 385, wherein the AAV capsid variant comprises an amino acid sequence comprising at least one, at least two, or at least three but no more than four modifications relative to the amino acid sequence of SPHSKA (SEQ ID NO: 941).
   387. The AAV particle of any one of embodiments 369, 371, or 385, wherein the AAV capsid variant comprises an amino acid sequence comprising at least one, at least two, or at least three but no more than four modifications relative to the amino acid sequence of HDSPHK (SEQ ID NO: 2).
   388. The AAV particle of any one of embodiments 369-371, 384, or 385, wherein the AAV capsid variant comprises an amino acid sequence comprising at least one, at least two, or at least three but no more than four modifications relative to the amino acid sequence of SPHKYG (SEQ ID NO: 966).
   389. The AAV particle of embodiment 370, wherein the AAV capsid variant comprises:
(i) an amino acid sequence comprising at least one, at least two, or at least three but no more than four modifications, relative to the amino acid sequence of KTERVSGSPHSKAQNQQT (SEQ ID NO: 3589);
(ii) an amino acid sequence comprising at least one, at least two, or at least three but no more than four modifications, relative to the amino acid sequence of KAEIGHDSPHKSGQNQQT (SEQ ID NO: 1754)
(iii) an amino acid sequence comprising at least one, at least two, or at least three but no more than four modifications, relative to the amino acid sequence of KTEKMSGSPHSKAQNQQT (SEQ ID NO: 3241);
(iv) an amino acid sequence comprising at least one, at least two, or at least three but no more than four modifications, relative to the amino acid sequence of KTINGHDSPHSKAQNLQT (SEQ ID NO: 4100): or
(v) an amino acid sequence comprising at least one, at least two, or at least three but no more than four modifications, relative to the amino acid sequence of KTVNGHDSPHSKAQNQQT (SEQ ID NO: 4062).
   390. The AAV particle of embodiment 369 or 371, wherein the AAV capsid variant comprises an amino acid sequence comprising at least one, at least two, or at least three but no more than four different amino acids relative to the amino acid sequence of any one of SEQ ID NOs: 2, 200, 201, 941, 943, 204, 208, 404, or 903-909.
   391. The AAV particle of any one of embodiments 369, 371, or 390, wherein the AAV capsid variant comprises an amino acid sequence comprising at least one, at least two, or at least three but no more than four different amino acids relative to the amino acid sequence of SPHSKA (SEQ ID NO: 941).
   392. The AAV particle of any one of embodiments 369, 371, or 390, wherein the AAV capsid variant comprises an amino acid sequence comprising at least one, at least two, or at least three but no more than four different amino acids relative to the amino acid sequence of HDSPHK (SEQ ID NO: 2).
   393. The AAV particle of any one of embodiments 369, 371, 384, or 390, wherein the AAV capsid variant comprises an amino acid sequence comprising at least one, at least two, or at least three but no more than four different amino acids relative to the amino acid sequence of SPHKYG (SEQ ID NO: 966).
   394. The AAV particle of embodiment 369, wherein the AAV capsid variant comprises:
(i) an amino acid sequence comprising at least one, at least two, or at least three but no more than four different amino acids relative to the amino acid sequence of KTERVSGSPHSKAQNQQT (SEQ ID NO: 3589);
(ii) an amino acid sequence comprising at least one, at least two, or at least three but no more than four different amino acids relative to the amino acid sequence of KAEIGHDSPHKSGQNQQT (SEQ ID NO: 1754);
(iii) an amino acid sequence comprising at least one, at least two, or at least three but no more than four different amino acids relative to the amino acid sequence of KTEKMSGSPHSKAQNQQT (SEQ ID NO: 3241);
(iv) an amino acid sequence comprising at least one, at least two, or at least three but no more than four different amino acids relative to the amino acid sequence of KTINGHDSPHSKAQNLQT (SEQ ID NO: 4100); or
(v) an amino acid sequence comprising at least one, at least two, or at least three but no more than four different amino acids relative to the amino acid sequence of KTVNGHDSPHSKAQNQQT (SEQ ID NO: 4062).
   395. The AAV particle of any one of embodiments 1-129, 269, 271, 375-388, or 390-394, wherein the AAV capsid variant comprises the amino acid sequence of any one of SEQ ID NOs: 2, 200, 201, 941, 943, 204, 208, 404, or 903-909.
   396. The AAV particle of any one of embodiments 295-297, 301-305, 313, 314, 318, 319, or 323, wherein the AAV capsid variant comprises the amino acid sequence of ERVSGSPHSKA (SEQ ID NO: 6399), optionally wherein the amino acid sequence is present immediately subsequent to position 450 and replaces positions 451-455 (e.g., 1451, N542, G453, S454, G455), numbered according to SEQ ID NO: 138.
   397. The AAV particle of any one of embodiments 369-371, 375-379, 385, 386, 389-391, or 394-3%, wherein the AAV capsid variant comprises the amino acid sequence of KTERVSGSPHSKAQNQQT (SEQ ID NO: 3589), optionally wherein the amino acid sequence is present immediately subsequent to position 448 and replaces positions 449-460 (e.g., K449, T450, 1451, N452, G453, S454, G455, Q456, N457, Q458, Q459, T460), numbered according to SEQ ID NO: 138.
   398. The AAV particle of any one of embodiments 269-371, 375, 380-383, 385, 387, 389, 390, 393, or 394, wherein the AAV capsid variant comprises the amino acid sequence of AEIGHDSPHKSG (SEQ ID NO: 6400), optionally wherein the amino acid sequence is present immediately subsequent to position 449 and replaces positions 450-455 (e.g., T450, 1451, N452, G453, S454, G455), numbered according to SEQ ID NO: 138.
   399. The AAV particle of any one of embodiments 369-371, 375, 380-383, 385, 387, 389, 390, 393, 394, or 398, wherein the AAV capsid variant comprises the amino acid sequence of KAEIGHDSPHKSGQNQQT (SEQ ID NO: 1754), optionally wherein the amino acid sequence is present immediately subsequent to position 448 and replaces positions 449-460 (e.g., K449, T450. 1451, N452, G453, S454, G455, Q456, N457, Q458, Q459. T460), numbered according to SEQ ID NO: 138.
   400. The AAV particle of any one of embodiments 369-371, 375-379, 390, 391, or 395, wherein the AAV capsid variant comprises the amino acid sequence of EKMSGSPHSKA (SEQ ID NO: 6401), optionally wherein the amino acid sequence is present immediately subsequent to position 450 and replaces positions 451-455 (e.g., 1451, N452, G453, S454, G455), numbered according to SEQ ID NO: 138.
   401. The AAV particle of any one of embodiments 369-371, 375-379, 390, 391, or 395, wherein the AAV capsid variant comprises the amino acid sequence of KTEKMSGSPHSKAQNQQT (SEQ ID NO: 3241), optionally wherein the amino acid sequence is present immediately subsequent to position 448 and replaces positions 449-460 (e.g., K449, T450, 1451, N452, G453, 5454, G455, Q456, N457, Q458, Q459, T460), numbered according to SEQ ID NO: 138.
   402. The AAV particle of any one of embodiments 369-371, 375-379, 390, 391, or 395, wherein the AAV capsid variant comprises the amino acid sequence of HDSPHSKAQNL (SEQ ID NO: 6402), optionally wherein the amino acid sequence is present immediately subsequent to position 453 and replaces positions 456-458 (e.g., Q456, N457, Q458), numbered according to SEQ ID NO: 138.
   403. The AAV particle of any one of embodiments 369-371, 375-379, 390, 391, or 395, wherein the AAV capsid variant comprises the amino acid sequence of KTINGHDSPHSKAQNLQT (SEQ ID NO: 4100), optionally wherein the amino acid sequence is present immediately subsequent to position 448 and replaces positions 449-460 (e.g., K449, T450, 1451, N452, G453, S454, G455, Q456, N457. Q458, Q459, T460), numbered according to SEQ ID NO: 138.
   404. The AAV particle of any one of embodiments 369-371, 375-379, 390, 391, or 395, wherein the AAV capsid variant comprises the amino acid sequence of VNGHDSPHSKA (SEQ ID NO: 6403), optionally wherein the amino acid sequence is present immediately subsequent to position 450 and replaces positions 451-455 (e.g., 1451, N452, G453, S454, G455), numbered according to SEQ ID NO: 138.
   405. The AAV particle of any one of embodiments 369-371, 375-379, 390, 391, or 395, wherein the AAV capsid variant comprises the amino acid sequence of KTVNGHDSPHSKAQNQQT (SEQ ID NO: 4062), optionally wherein the amino acid sequence is present immediately subsequent to position 448 and replaces positions 449-460 (e.g., K449, T450, I451, N452, G453, 5454, G455, Q456, N457, Q458, Q459. T460), numbered according to SEQ ID NO: 138.
   406. The AAV particle of any one of embodiments 1-23, 26-29, 32, 3543, 46-51, 54-72, 69-89, 91. 94-99, 102-104, 107, 110-112, 115-129, 168-202, 240-247, 249, 250, 253-263, 266, 272-281, 286, 288, 291-299, 327-363, 369-371, 375-379, 385, 386, 390, 391, or 395, wherein the AAV capsid variant comprises an amino acid sequence encoded by: the nucleotide sequence of SEQ ID NO: 942; a nucleotide sequence comprising at least one, at least two, at least three, at least four, at least five, at least six, or at least seven modifications, e.g., substitutions (e.g., conservative substitutions), but no more than ten modifications, e.g., substitutions (e.g., conservative substitutions), relative to the nucleotide sequence of SEQ ID NO: 942; or a nucleotide sequence comprising at least one, at least two, at least three, at least four, at least five, at least six, or at least seven, but no more than ten different nucleotides relative to the nucleotide sequence of SEQ ID NO: 942.
   407. The AAV particle of any one of embodiments 1-9, 11, 12-22, 24, 26, 28, 30, 33, 3542, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, or 395, wherein the AAV capsid variant comprises an amino acid sequence encoded by: the nucleotide sequence of SEQ ID NO: 3; a nucleotide sequence comprising at least one, at least two, at least three, at least four, at least five, at least six, or at least seven modifications, e.g., substitutions (e.g., conservative substitutions), but no more than ten modifications, e.g., substitutions (e.g., conservative substitutions), relative to the nucleotide sequence of SEQ ID NO: 3: or a nucleotide sequence comprising at least at least one, at least two, at least three, at least four, at least five, at least six, or at least seven, but no more than ten different nucleotides relative to the nucleotide sequence of SEQ ID NO: 3.
   408. The AAV particle of any one of embodiments 1-23, 26-29, 32, 3543, 46-51, 54-72, 69-89, 91, 94-99, 102-104, 107, 110-112, 115-129, 168-202, 240-247, 249, 250, 253-263, 266, 272-281, 286, 288, 291-299, 327-363, 369-371, 375-379, 385, 386, 390, 391, 395, or 406, wherein the nucleotide sequence encoding the capsid variant comprises the nucleotide sequence of SEQ ID NO: 942; a nucleotide sequence comprising at least one, at least two, at least three, at least four, at least five, at least six, or at least seven modifications, e.g., substitutions (e.g., conservative substitutions), but no more than ten modifications, e.g., substitutions (e.g., conservative substitutions), relative to the nucleotide sequence of SEQ ID NO: 942: or a nucleotide sequence comprising at least one, at least two, at least three, at least four, at least five, at least six, or at least seven, but no more than ten different nucleotides relative to the nucleotide sequence of SEQ ID NO: 942.
   409. The AAV particle of any one of embodiments 1-9, 11, 12-22, 24, 26, 28, 30, 33, 35-42, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, or 407, wherein the nucleotide sequence encoding the capsid variant comprises the nucleotide sequence of SEQ ID NO: 3, a nucleotide sequence comprising at least one, at least two, at least three, at least four, at least five, at least six, or at least seven modifications, e.g., substitutions (e.g., conservative substitutions), but no more than ten modifications, e.g., substitutions (e.g., conservative substitutions), relative to the nucleotide sequence of SEQ ID NO: 3; or a nucleotide sequence comprising at least one, at least two, at least three, at least four, at least five, at least six, or at least seven, but no more than ten different nucleotides relative to the nucleotide sequence of SEQ ID NO: 3.
   410. The AAV particle of any one of embodiments 369-409, wherein the amino acid sequence is present in loop TV, e.g., relative to the amino acid sequence of SEQ ID NO: 138.
   411. The AAV particle of any one of embodiments 369-410, wherein the amino acid sequence is pesent immediately subsequent to position 448, 449, 450, 451, 452, 453, 454, or 455, numbered according to SEQ ID NO: 138.
   412. The AAV particle of any one of embodiments 369-411, wherein the amino acid sequence replaces amino acids 449, 450, 451, 452, 453, 454, 455, 456, 457, 458, 459, and/or 460 (e.g., K449, T450, 1451, N452, G453, S454, G455, Q456, N457, Q458, Q459, and/or T460), numbered according SEQ ID NO: 138.
   413. The AAV particle of any one of embodiments 369-412, wherein the amino acid sequence is present immediately subsequent to position 455, numbered according to SEQ ID NO: 138.
   414. The AAV particle of any one of embodiments 369-413, wherein the amino acid sequence is present immediately subsequent to position 453, numbered according SEQ ID NO: 138.
   415. The AAV particle of any one of embodiments 1-23, 26-29, 32, 35-43, 46-51, 54-72, 69-89, 91, 94-99, 102-104, 107, 110-112, 115-129, 168-202, 02, 240-247,249, 250, 253-263, 266, 272-281, 286, 288, 291-299, 327-363, 369-371, 375-379, 385, 386, 390, 391, 395, 406, 408, or 410-413, wherein the AAV capsid variant comprises the amino acid sequence of SPHSKA (SEQ ID NO: 941), wherein the amino acid sequence is present immediately subsequent to position 455, according to SEQ ID NO: 138.
   416. The AAV particle of any one of embodiments 1-23, 26-29, 32, 3543, 46-51, 54-72, 69-89, 91, 94-99, 102-104, 107, 110-112, 115-129, 168-202, 02, 240-247, 249, 250, 253-263, 266, 272-281, 286, 288, 291-299, 327-363, 369-371, 375-379, 385, 386, 390, 391, 395, 406, 408, 410-413, or 415, wherein the AAV capsid variant the amino acid sequence of SPHSKA (SEQ ID NO: 941), wherein the amino acid sequence is present immediately subsequent to position 455, numbered according to SEQ ID NO: 981.
   417. The AAV particle of embodiment 415 or 416, wherein the AAV capsid variant further comprises an amino acid other than I at position 451, an amino acid other than N at position 452, and an amino acid other than G at position 453, numbered according to any one of SEQ ID NOs: 36, 138, or 981.
   418. The AAV particle of any one of embodiments 415-417, wherein the AAV capsid variant further comprises E at position 451, R at position 452, and V at position 453, numbered according to any one of SEQ ID NOs: 36, 138, or 981.
   419. The AAV particle of any one of embodiments 415418, wherein the AAV capsid variant further comprises the substitutions 1451E, N452R, and G453V, numbered according to any one of SEQ ID NOs: 36, 138, or 981.
   420. The AAV particle of any one of embodiments 415-419, wherein the AAV capsid variant comprises:
(i) E at position 451. R at position 452, and V at position 453, numbered according to any one of SEQ ID NOs: 36, 138, or 981; and
(ii) the amino acid sequence of SPHSKA (SEQ ID NO: 941), wherein the amino acid sequence is present immediately subsequent to position 455, numbered according to any one of SEQ ID NOs: 36, 138, or 981.
   421. The AAV particle of embodiment 415 or 416, wherein the AAV capsid variant further comprises an amino acid other than I at position 451, an amino acid other than N at position 452, and/or G at position 453, numbered according to SEQ ID NO: 39 or 138.
   422. The AAV particle of any one of embodiments 415, 416, or 421, wherein the AAV capsid variant further comprises E at position 451, K at position 452, and/or M at position 453, numbered according to SEQ ID NO: 138 or 39.
   423. The AAV particle of any one of embodiments 415, 416, 421, or 422, wherein the AAV capsid variant further comprises the substitutions 1451E, N452K, and G453M, numbered according to SEQ ID NO: 39 or 138. 424. The AAV particle of any one of embodiments 415, 416, or 421423, wherein the AAV capsid variant comprises:
(i) E at position 451, K at position 452, and M at position 453, numbered according to SEQ ID NO: 39 or 138; and
(ii) the amino acid sequence of SPHSKA (SEQ ID NO: 941), wherein the amino acid sequence is present immediately subsequent to position 455, numbered according to SEQ ID NO: 39 or 138.
   425. The AAV particle of embodiment 415 or 416, wherein the AAV capsid variant further comprises an amino acid other than S at position 454, an amino acid other than G at position 455, and/or Q at position 458, numbered according to SEQ ID NO: 138.
   426. The AAV particle of any one of embodiments 415, 416, or 425, wherein the AAV capsid variant further comprises H at position 454. D at position 455, and/or L at position 458, numbered according to SEQ ID NO: 138.
   427. The AAV particle of any one of embodiments 415, 416, 425, or 426, wherein the AAV capsid variant further comprises the substitutions S454H, G455D, and Q458L, numbered according to SEQ ID NO: 138.
   428. The AAV particle of any one of embodiments 415, 416, or 425-427, wherein the AAV capsid variant comprises:
(i) H at position 454, D at position 455, and/or L at position 458, numbered according to SEQ ID NO: 138; and
(ii) the amino acid sequence of SPHSKA (SEQ ID NO: 941), wherein the amino acid sequence is present immediately subsequent to position 455, numbered according to SEQ ID NO: 138.
   429. The AAV particle of embodiment 415 or 416, wherein the AAV capsid variant further comprises an amino acid other than I at position 451, an amino acid other than S at position 454, and/or an amino acid other than G at position 455, numbered according to SEQ ID NO: 52 or 138.
   430. The AAV particle of any one of embodiments 415, 416, or 429, wherein the AAV capsid variant further comprises V at position 451, H at position 454, and/or D at position 455, numbered according to SEQ ID NO: 52 or 138.
   431. The AAV particle of any one of embodiments 415, 416, 429, or 430, wherein the AAV capsid variant further comprises the substitutions 145 IV, S454H, and/or G455D, numbered according to SEQ ID NO: 52 or 138.
   432. The AAV particle of any one of embodiments 415, 416, or 429-431, wherein the AAV capsid variant comprises:
(i) V at position 451, H at position 454, and/or D at position 455, numbered according to SEQ ID NO: 52 or 138; and
(ii) the amino acid sequence of SPHSKA (SEQ ID NO: 941), wherein the amino acid sequence is present immediately subsequent to position 455, numbered according to SEQ ID NO: 52 or 138.

433. The AAV particle of any one of embodiments 1-9, 11, 12-22, 24, 26, 28, 30, 33, 3542, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409-412, or 414, wherein the AAV capsid variant comprises the amino acid sequence of HDSPHK (SEQ ID NO: 2), wherein the amino acid sequence is present immediately subsequent to position 453, numbered according to the amino acid sequence of SEQ ID NO: 138.

434. The AAV particle of any one of embodiments 1-9, 11, 12-22, 24, 26, 28, 30, 33, 3542, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409412. 414, or 433, wherein the AAV capsid variant comprises the amino acid sequence of HDSPHK (SEQ ID NO: 2), wherein the amino acid sequence is present immediately subsequent to position 453, numbered according to the amino acid sequence of SEQ ID NO: 982.
435. The AAV particle of any one of embodiments 1-9, 11, 12-22, 24, 26, 28, 30, 33, 35-42, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409412, 414, 433, or 434, wherein the AAV capsid variant comprises the amino acid sequence of SPHKSG (SEQ ID NO: 946), wherein the amino acid sequence is present immediately subsequent to position 455, numbered according to the amino acid sequence of SEQ ID NO: 982.
436. The AAV particle of any one of embodiments 369-435, wherein the AAV capsid variant comprises:
(i) the amino acid sequence of HDSPHSKA (SEQ ID NO: 4486), which is present immediately subsequent to position 453; and
(ii) a deletion of amino acids SG at position 454 and 455;
wherein (i) and (ii) are numbered according to SEQ ID NO: 138.
   437. The AAV particle of any one of embodiments 369-436, wherein the AAV capsid variant comprises the amino acids HD at position 454 and 455, and further comprises the amino acid sequence of SPHSKA (SEQ ID NO: 941), which is present immediately subsequent to position 455, numbered according to SEQ ID NO: 138.
   438. The AAV particle of any one of embodiments 433-435, wherein the AAV capsid variant further comprises an amino acid other than T at position 450, an amino acid other than I at position 451, and an amino acid other than N at position 452, numbered according to SEQ ID NO: 138 or 982.
   439. The AAV particle of any one of embodiments 433-435 or 438, wherein the AAV capsid variant further comprises A at position 450. E at position 451, and I at position 452, numbered according to SEQ ID NO: 138 or 982.
   440. The AAV particle of any one of embodiments 433-435, 438, or 439, wherein the AAV capsid variant further comprises the substitutions T450A, 1451E, and N452I, numbered according to SEQ ID NO: 138 or 982.
   441. The AAV particle of any one of embodiments 433, 434, or 438-440, wherein the AAV capsid variant comprises:
(i) A at position 450, E at position 451, and I at position 452, numbered according to SEQ ID NO: 138 or 982; and
(ii) the amino acid sequence of HDSPHK (SEQ ID NO: 2), which is present immediately subsequent to positions 453, numbered according to SEQ ID NO: 138 or 982.
   442. The AAV particle of any one of embodiments 1-22, 25-27, 31, 34-42, 45-50, 53-63, 69, 79, 83-86, 91-98, 102, 103, 110, 111, 118-129, 369-371, 384, 385, 390, 393, 395, 410-413, wherein the AAV capsid variant comprises the amino acid sequence of SPHKYG (SEQ ID NO: 966), wherein the amino acid sequence is present immediately subsequent to position 455, numbered according to the amino acid sequence of SEQ ID NO: 138.
   443. An adeno-associated virus (AAV) particle comprising an AAV capsid variant comprising the amino acid sequence of HDSPHK (SEQ ID NO: 2), wherein the amino acid sequence is present immediately subsequent to position 453, numbered according to the amino acid sequence of SEQ ID NO: 982, wherein the AAV particle further comprises a nucleic acid encoding a syntaxin-binding protein 1 (STXBP1) protein (e.g., a human STXBP1 protein).
   444. An adeno-associated virus (AAV) particle comprising an AAV capsid variant and a nucleic acid encoding a syntaxin-binding protein 1 (STXBP1) protein (e.g., a human STXBP1 protein), wherein the AAV capsid variant comprises the amino acid sequence of SPHSKA (SEQ ID NO: 941), wherein the amino acid sequence is present immediately subsequent to position 455, numbered according to the amino acid sequence of SEQ ID NO: 981.
   445. An adeno-associated virus (AAV) particle comprising an AAV capsid variant comprising the amino acid sequence of HDSPHK (SEQ ID NO: 2), wherein the amino acid sequence is present immediately subsequent to position 453, numbered according to the amino acid sequence of SEQ ID NO: 37, and optionally further comprising:
(i) one, two, or all of an amino acid other than T at position 450, an amino acid other than I at position 541, and/or an amino acid other than N at position 452, numbered according to SEQ ID NO: 138 or 37;
(ii) one, two, or all of A at position 450, E at position 451, and/or I at position 452, numbered according to SEQ ID NO: 138 or 37;
wherein the AAV particle further comprises a nucleic acid encoding a syntaxin-binding protein 1 (STXBP1) protein (e.g., a human STXBP1 protein).
   446. An adeno-associated virus (AAV) particle comprising an AAV capsid variant comprising the amino acid sequence of SPHSKA (SEQ ID NO: 941), wherein the amino acid sequence is present immediately subsequent to position 455, numbered according to the amino acid sequence of any one of SEQ ID NO: 36, 38-55, 57, or 59, wherein the AAV particle further comprises a nucleic acid encoding a syntaxin-binding protein 1 (STXBP1) protein (e.g., a human STXBP1 protein).
   447. The AAV particle of any one of the preceding embodiments, wherein the AAV capsid variant further comprises:
(i) a modification in loop I, II, VI and/or VIII; and/or
(ii) a substitution at position K449, e.g., a K449R substitution, numbered according to SEQ ID NO: 138.
   448. The AAV particle of any one of the preceding embodiments, wherein the AAV capsid variant comprises an amino acid sequence comprising at least one, at least two, or at least three modifications, e.g., substitutions (e.g., conservative substitutions), but not more than 30, not more than 20, or not more than 10 modifications, e.g., substitutions (e.g., conservative substitutions), relative to the amino acid sequence of SEQ ID NO: 138.
   449. The AAV particle of any one of the preceding embodiments, wherein the AAV capsid variant comprises an amino acid sequence comprising at least one, at least two, or at least three, but no more than 30, not more than 20, or not more than 10 different amino acids relative to the amino acid sequence of SEQ ID NO: 138.
   450. The AAV particle of any one of the preceding embodiments, wherein the AAV capsid variant comprises an amino acid sequence with at least 80% (e.g., at least 80%, at least 85%, at least 90%, at least 95%, at least 996%, at least 97%, at least 98%, or at least 99%) sequence identity to SEQ ID NO: 138.
   451. The AAV particle of any one of the preceding embodiments, wherein the AAV capsid variant comprises an amino acid sequence with at least 98% identity to SEQ ID NO: 138.
   452. The AAV particle of any one of the preceding embodiments, wherein the AAV capsid variant comprises an amino acid sequence encoded by a sequence with at least 80% (e.g., at least 80%, at least 85%, at least 90%, at least 95%, at least 96%, at least 97%, at least 98%, or at least 99%) sequence identity to SEQ ID NO: 137.
   453. The AAV particle of any one of the preceding embodiments, wherein the nucleotide sequence encoding the capsid variant comprises a sequence with at least 80% (e.g., at least 80%, at least 85%, at least 90%, at least 95%, at least 96%, at least 97%, at least 98%, or at least 99%) sequence identity to SEQ ID NO: 137.
   454. The AAV particle of any one of the preceding embodiments, wherein the AAV capsid variant comprises a VP1 protein, a VP2 protein, a VP3 protein, or a combination thereof.
   455. The AAV particle of any one of embodiments 1454, wherein the AAV capsid variant comprises the amino acid sequence corresponding to positions 138-742, e.g., a VP2, of SEQ ID NO: 981, 982, 36, or 4, or a sequence with at least 80% (e.g., at least 80%, at least 85%, at least 90%, at least 95%, at least 96%, at least 97%, at least 98%, or at least 99%) sequence identity thereto.
   456. The AAV particle of any one of embodiments 1-455, wherein the AAV capsid variant comprises the amino acid sequence corresponding to positions 203-742, e.g., a VP3, of SEQ ID NO: 981, 982, 36, or 4, or a sequence with at least 80% (e.g., at least 80%, at least 85%, at least 90%, at least 95%, at least 96%, at least 97%, at least 98%, or at least 99%) sequence identity thereto.
   457. The AAV particle of any one of embodiments 1-456, wherein the AAV capsid variant comprises an amino acid sequence with at least 80% (e.g., at least 80%, at least 85%, at least 90%, at least 95%, at least 96%, at least 97%, at least 98%, or at least 99%) sequence identity to SEQ ID NO: 138.
   458. The AAV particle of any one of embodiments 1-457, wherein the AAV capsid variant comprises an amino acid sequence with at least 80% (e.g., at least 80%, at least 85%, at least 90%, at least 95%, at least 96%, at least 97%, at least 98%, or at least 99%) sequence identity to SEQ ID NO: 138.
   459. The AAV particle of any one of embodiments 1-23, 26-29, 32, 35-43, 46-51, 54-72, 69-89, 91, 94-99, 102-104, 107, 110-112, 115-129, 168-202, 240-247, 249, 250, 253-263, 266, 272-281, 286, 288, 291-299, 327-363, 369-371, 375-379, 385, 386, 390, 391, 395, 406, 408, 410-413, 415432, 444, or 446-458, wherein the AAV capsid variant comprises an amino acid sequence comprising at least 3, at least 4, at least 5, or at least 6 consecutive amino acids from the amino acid sequence of SPHSKA (SEQ ID NO: 941), wherein:
(i) the at least 3 consecutive amino acids comprise SPH;
(ii) the at least 4 consecutive amino acids comprise SPHS (SEQ ID NO: 4700);
(iii) the at least 5 consecutive amino acids comprise SPHSK (SEQ ID NO: 4701); or
(iv) the at least 6 consecutive amino acids comprise SPHSKA (SEQ ID NO: 941);
wherein the AAV capsid variant comprises: (a) a VP1 protein comprising the amino acid sequence of SEQ ID NO: 981; (b) a VP2 protein comprising the amino acid sequence of positions 138-742 of SEQ ID NO: 981; (c) a VP3 protein comprising the amino acid sequence of positions 203-742 of SEQ ID NO: 981: or (d) an amino acid sequence with at least 90% (e.g., at least 90%, at least 95%, at least 96%, at least 97%, at least 98%, or at least 99%) sequence identity to any of the amino acid sequences in (a)-(c).
   460. The AAV particle of any one of embodiments 1-23, 26-29, 32, 3543, 46-51, 54-72, 69-89, 91, 94-99, 102-104, 107, 110-112, 115-129, 168-202, 240-247, 249, 250.253-263, 266, 272-281, 286, 288, 291-299, 327-363, 369-371, 375-379, 385, 386, 390, 391, 395, 406, 408, 410-413, 415-432, 444, or 446459, wherein the AAV capsid variant comprises an amino acid sequence comprising at least 3, at least 4, at least 5, or at least 6 consecutive amino acids from the amino acid sequence of SPHSKA (SEQ ID NO: 941), wherein:
(i) the at least 3 consecutive amino acids comprise SPH;
(ii) the at least 4 consecutive amino acids comprise SPHS (SEQ ID NO: 47M00);
(iii) the at least 5 consecutive amino acids comprise SPHSK (SEQ ID NO: 4701): or
(iv) the at least 6 consecutive amino acids comprise SPHSKA (SEQ ID NO: 941); wherein the AAV capsid variant comprises an amino acid sequence at least 90/u (e.g., at least 90%, at least 95%, at least 96%, at least 97%, at least 98%, or at least 99%) to the amino acid sequence of SEQ ID NO: 981.
   461. The AAV particle of any one of embodiments 1-23, 26-29, 32, 3543, 46-51, 54-72, 69-89, 91, 94-99, 102-104, 107, 110-112, 115-129, 168-202, 240-247, 249, 250, 253-263, 266, 272-281, 286, 288, 291-299, 327-363, 369-371, 375-379, 385, 386, 390, 391, 395, 406, 408, 410-413, 415-432, 444, or 446460, wherein the AAV capsid variant comprises one or two, but no more than three substitutions relative to the amino acid sequence of SPHSKA (SEQ ID NO: 941), wherein the AAV capsid variant comprises:
(a) a VP1 protein comprising the amino acid sequence of SEQ ID NO: 981; (b) a VP2 protein comprising the amino acid sequence of positions 138-742 of SEQ ID NO: 981;
(c) a VP3 protein comprising the amino acid sequence of positions 203-742 of SEQ ID NO: 981; or
(d) an amino acid sequence with at least 90% (e.g., at least 90%, at least 95%, at least 96%, at least 97%, at least 98/6, or at least 99%) sequence identity to any of the amino acid sequences in (a)-(c).
   462. The AAV particle of any one of embodiments 1-23, 26-29, 32, 3543, 46-51, 54-72, 69-89, 91, 94-99, 102-104, 107, 110-112, 115-129, 168-202, 240-247, 249, 250, 253-263, 266, 272-281, 286, 288, 291-299, 327-363, 369-371, 375-379, 385, 386, 390, 391, 395, 406, 408, 410-413, 415-432, 444, or 446-461, wherein the AAV capsid variant comprises one or two, but no more than three substitutions relative to the amino acid sequence of SPHSKA (SEQ ID NO: 941), wherein the AAV capsid variant comprises an amino acid sequence at least 90% (e.g., at least 90%, at least 95%, at least 96%, at least 97%, at least 98%, or at least 99%) identical to the amino acid sequence of SEQ ID NO: 138 or SEQ ID NO: 981.
   463. The AAV particle of any one of embodiments 459-462, wherein the amino acid sequence is present immediately subsequent to position 455, numbered according to SEQ ID NO: 138 or 981.
   464. The AAV particle of any one of embodiments 1-9, 11, 12-22, 24, 26, 28, 30, 33, 35-42, 44, 46-50,52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409-412, 414, 433-435, 438-441, 443, 445, or 447-458, wherein the AAV capsid variant an amino acid sequence comprising at least 3, at least 4, at least 5, or at least 6 consecutive amino acids from the amino acid sequence of HDSPHK (SEQ ID NO: 2), wherein:
(i) the at least 3 consecutive amino acids comprise HDS;
(ii) the at least 4 consecutive amino acids comprise HDSP (SEQ ID NO: 4702);
(iii) the at least 5 consecutive amino acids comprise HDSPH (SEQ ID NO: 4703): or
(iv) the at least 6 consecutive amino acids comprise HDSPHK (SEQ ID NO: 2);
wherein the AAV capsid variant comprises: (a) a VP1 protein comprising the amino acid sequence of SEQ ID NO: 982; (b) a VP2 protein comprising the amino acid sequence of positions 138-742 of SEQ ID NO: 982: (c) a VP3 protein comprising the amino acid sequence of positions 203-742 of SEQ ID NO: 982; or (d) an amino acid sequence with at least 90% (e.g., at least 90%, at least 95%, at least 96%, at least 97%, at least 98%, or at least 99%) sequence identity to any of the amino acid sequences in (a)-(c).
   465. The AAV particle of any one of embodiments 1-9, 11, 12-22, 24, 26, 28, 30, 33, 3542, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409-412, 414, 433-435, 438441, 443, 445, 447458, or 464, wherein the AAV capsid variant comprises an amino acid sequence comprising at least 3, at least 4, at least 5, or at least 6 consecutive amino acids from the amino acid sequence of HDSPHK (SEQ ID NO: 2), wherein:
(i) the at least 3 consecutive amino acids comprise HDS;
(ii) the at least 4 consecutive amino acids comprise HDSP (SEQ ID NO: 4702);
(iii) the at least 5 consecutive amino acids comprise HDSPH (SEQ ID NO: 4703): or
(iv) the at least 6 consecutive amino acids comprise HDSPHK (SEQ ID NO: 2);
wherein the AAV capsid variant comprises an amino acid sequence at least 90% (e.g., at least 90%, at least 95%, at least 96%, at least 97%, at least 98%, or at least 99%) identical to the amino acid sequence of SEQ ID NO: 982.
   466. The AAV particle of any one of embodiments 1-9, 11, 12-22, 24, 26, 28, 30, 33, 3542, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409412. 414, 433435, 438-441, 443, 445, 447-458, 464, or 465, wherein the AAV capsid variant comprises one or two, but no more than three substitutions relative to the amino acid sequence of HDSPHK (SEQ ID NO: 2), wherein the AAV capsid variant comprises:
(a) a VP1 protein comprising the amino acid sequence of SEQ ID NO: 982;
(b) a VP2 protein comprising the amino acid sequence of positions 138-742 of SEQ ID NO: 982;
(c) a VP3 protein comprising the amino acid sequence of positions 203-742 of SEQ ID NO: 982: or
(d) an amino acid sequence with at least 90% (e.g., at least 90%, at least 95%, at least 96%, at least 97%, at least 98%, or at least 99%) sequence identity to any of the amino acid sequences in (a)-(c).
   467. The AAV particle of any one of embodiments 1-9, 11, 12-22, 24, 26, 28, 30, 33, 35-42, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409-412, 414, 433-435, 438-441, 443, 445, 447-458, or 464-466, wherein the AAV capsid variant comprises one or two, but no more than three substitutions relative to the amino acid sequence of HDSPHK (SEQ ID NO: 2), wherein the AAV capsid variant comprises an amino acid sequence at least 90% (e.g., at least 90%, at least 95%, at least 96%, at least 97%, at least 98%, or at least 99%) identical to the amino acid sequence of SEQ ID NO: 982.
   468. The AAV particle of any one of embodiments 464468, wherein the amino acid sequence is present immediately subsequent to position 453, numbered according to SEQ ID NO: 138 or 982.
   469. The AAV particle of any one of embodiments 1468, wherein the AAV capsid variant comprises the amino acid sequence of SEQ ID NO: 981 or 982, or an amino acid sequence with at least 80% (e.g., at least 80%, at least 85%, at least 90%, at least 95%, at least 96%, at least 97%, at least 98%, or at least 99/6) sequence identity thereto.
   470. The AAV particle of any one of embodiments 1-469, wherein the AAV capsid variant comprises an amino acid sequence comprising at least one, at least two or at least three modifications, e.g., substitutions (e.g., conservative substitutions), but not more than 30, not more than 20 or not more than 10 modifications, e.g., substitutions (e.g., conservative substitutions), relative to the amino acid sequence of SEQ ID NO: 981 or 982.
   471. The AAV particle of any one of embodiments. 1470, wherein the AAV capsid variant comprises an amino acid sequence comprising at least one, at least two or at least three, but not more than 30, not more than 20 or not more than 10 different amino acids, relative to the amino acid sequence of SEQ ID NO: 981 or 982.
   472. The AAV particle of any one of embodiments 1-23, 26-29, 32, 3543, 46-51, 54-72, 69-89, 91. 94-99, 102-104, 107, 110-112, 115-129, 168-202, 240-247, 249, 250, 253-263, 266, 272-281, 286, 288, 291-299, 327-363, 369-371, 375-379, 385, 386, 390, 391, 395, 406, 408, 410-413, 415432, 444, 446463, or 469-471, wherein the AAV capsid variant comprises the amino acid sequence of SEQ ID NO: 981, or an amino acid sequence with at least 80% (e.g., at least 80%, at least 85%, at least 9%, at least 95%, at least 96%, at least 97%, at least 98%, or at least 99%) sequence identity thereto.
   473. The AAV particle of any one of embodiments 1-23, 26-29, 32, 35-43, 46-51, 54-72, 69-89, 91, 94-99, 102-104, 107, 110-112, 115-129, 168-202, 240-247, 249, 250, 253-263, 266, 272-281, 286, 288, 291-299, 327-363, 369-371, 375-379, 385, 386, 390, 391, 395, 406, 408, 410-413, 415432, 444, 446-463, or 469472, wherein the AAV capsid variant comprises an amino acid sequence comprising at least one, at least two or at least three modifications, e.g., substitutions (e.g., conservative substitutions), but not more than 30, not more than 20 or not more than 10 modifications, e.g., substitutions (e.g., conservative substitutions), relative to the amino acid sequence of SEQ ID NO: 981.
   474. The AAV particle of any one of embodiments 1-23, 26-29, 32, 3543, 46-51, 54-72, 69-89, 91, 94-99, 102-104, 107, 110-112, 115-129, 168-202, 240-247, 249, 250, 253-263, 266, 272-281, 286, 288, 291-299, 327-363, 369-371, 375-379, 385, 386, 390, 391, 395, 406, 408, 410-413, 415432, 444, 446463, or 469473, wherein the AAV capsid variant comprises an amino acid sequence comprising at least one, at least two or at least three, but not more than 30, not more than 20 or not more than 10 different amino acids, relative to the amino acid sequence of SEQ ID NO: 981.
   475. The AAV particle of any one of embodiments 1-9, 11, 12-22, 24, 26, 28, 30, 33, 3542, 44,46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409-412, 414, 433-435, 438-441, 443, 445, 447458, or 464471, wherein the AAV capsid variant comprises the amino acid sequence of SEQ ID NO: 982, or an amino acid sequence with at least 80% (e.g., at least 80%, at least 85%, at least 90%, at least 95%, at least 96%, at least 97%, at least 98%, or at least 99%) sequence identity thereto.
   476. The AAV particle of any one of embodiments 1-9, 11, 12-22, 24, 26, 28, 30, 33, 35-42, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409412, 414, 433435, 438441, 443, 445, 447458, 464471, or 475, wherein the AAV capsid variant comprises an amino acid sequence comprising at least one, at least two or at least three modifications, e.g., substitutions (e.g., conservative substitutions), but not more than 30, not more than 20 or not more than 10 modifications, e.g., substitutions (e.g., conservative substitutions), relative to the amino acid sequence of SEQ ID NO: 982.
   477. The AAV particle of any one of embodiments 1-9, 11, 12-22, 24, 26, 28, 30, 33, 35-42, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409-412, 414, 433435, 438441, 443, 445, 447-458, 464471, 475, or 476, wherein the AAV capsid variant comprises an amino acid sequence comprising at least one, at least two or at least three, but not more than 30, not more than 20 or not more than 10 different amino acids, relative to the amino acid sequence of SEQ ID NO: 982.
   478. The AAV particle of any one of embodiments 1-477, wherein the AAV capsid variant comprises an amino acid sequence encoded by the nucleotide sequence of SEQ ID NO: 983 or 984, or a nucleotide sequence with at least 80% (e.g., at least 80%, at least 85%, at least 90%, at least 95%, at least 696%, at least 97%, at least 98%, or at least 99%) sequence identity thereto.
   479. The AAV particle of any one of embodiments 1478, wherein the nucleotide sequence encoding the capsid variant comprises the nucleotide sequence of SEQ ID NOs: 983 or 984, or a nucleotide sequence with at least 80% (e.g., at least 80%, at least 85%, at least 90%, at least 95%, at least %*⁰%0, at least 97%, at least 98%, or at least 99%) sequence identity thereto.
   480. The AAV particle of any one of embodiments 1-23, 26-29, 32, 35-43, 46-51, 54-72, 69-89, 91, 94-99, 102-104, 107, 110-112, 115-129, 168-202, 02, 240-247, 249, 250, 253-263, 266, 272-281, 286, 288, 291-299, 327-363, 369-371, 375-379, 385, 386, 390, 391, 395, 406, 408, 410413, 415432, 444, 446463, 469474, 478, or 479, wherein the nucleotide sequence encoding the capsid variant comprises the nucleotide sequence of SEQ ID NO: 983, or a nucleotide sequence with at least 80% (e.g., at least 80%, at least 85%, at least 90%, at least 95%, at least 96%, at least 97%, at least 98%, or at least 990) sequence identity thereto.
   481. The AAV particle of any one of embodiments 1-9, 11, 12-22, 24, 26, 28, 30, 33, 35-42, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409412, 414, 433435, 438441, 443, 445, 447-458, 464471, or 475479, wherein the nucleotide sequence encoding the capsid variant comprises the nucleotide sequence of SEQ ID NO: 984, or a nucleotide sequence with at least 80% (e.g., at least 80%, at least 85%, at least 90%, at least 95%, at least 96%, at least 97%, at least 98%, or at least 99%) sequence identity thereto.
   482. The AAV particle of any one of the preceding embodiments, wherein the nucleotide sequence encoding the capsid variant is codon optimized.
   483. An AAV particle of any one of embodiments 1-23, 26-29, 32, 3543, 46-51, 54-72, 69-89, 91, 94-99, 102-104, 107, 110-112, 115-129, 168-202, 02, 240-247, 249, 250, 253-263, 266, 272-281, 286. 288, 291-299, 327-363, 369-371, 375-379, 385, 386, 390, 391, 395, 406, 408, 410413, 415432, 444. 446463, 469-474, 478-480, or 482, and further comprising an amino acid sequence at least 95% identical to SEQ ID NO: 981.
   484. An adeno-associated virus (AAV) particle comprising an AAV capsid variant and a nucleic acid encoding a syntaxin-binding protein 1 (STXBP1) protein (e.g., a human STXBP1 protein), wherein the AAV capsid variant comprises the amino acid sequence of SEQ ID NO: 981.
   485. The AAV particle of embodiment 483 or 484, wherein the nucleotide sequence encoding the AAV capsid variant comprises the nucleotide sequence of SEQ ID NO: 983, or a nucleotide sequence at least 90%, at least 95%, or at least 99% identical thereto.
   486. The AAV particle of any one of embodiments 1-9, 11, 12-22, 24, 26, 28, 30, 33, 3542, 44, 46-50, 52, 54-77, 83-88, 90-97, 100-103, 105, 110, 111, 113-129, 203-248, 251, 252, 254-257, 260, 261, 264-280, 283-287, 289-299, 327-363, 369, 369, 371, 380-383, 385, 386, 390, 392, 395, 407, 409-412, 414, 433-435, 438-441, 443, 445, 447-458, 464-471, 475479, 481, or 482, and further comprising an amino acid sequence at least 95% identical to SEQ ID NO: 982.
   487. An AAV particle comprising an AAV capsid variant and a nucleic acid encoding a syntaxin-binding protein 1 (STXBP1) protein (e.g., a human STXBP1 protein), wherein the AAV capsid variant comprises the amino acid sequence of SEQ ID NO: 982.
   488. The AAV particle of embodiment 486 or 487, wherein the nucleotide sequence encoding the AAV capsid variant comprises the nucleotide sequence of SEQ ID NO: 984, or a nucleotide sequence at least 90%, at least 95%, or at least 99% identical thereto.
   489. An adeno-associated virus (AAV) particle comprising an AAV capsid variant and a nucleic acid encoding a syntaxin-binding protein 1 (STXBP1) protein (e.g., a human STXBP1 protein), wherein the AAV capsid variant comprises an amino acid sequence encoded by the nucleotide sequence of SEQ ID NOs: 983 or 984, or a nucleotide sequence at least 95% identical thereto.
   490. An adeno-associated virus (AAV) particle comprising an AAV capsid variant and a nucleic acid encoding a syntaxin-binding protein 1 (STXBP1) protein (e.g., a human STXBP1 protein), wherein the AAV capsid variant comprises the amino acid sequence of any one of SEQ ID NOs: 4 or 36-59, optionally wherein the AAV capsid variant comprises the amino acid sequence of SEQ ID NO: 4 or 36.
   491. An adeno-associated virus (AAV) particle comprising an AAV capsid variant and a nucleic acid encoding a syntaxin-binding protein 1 (STXBP1) protein (e.g., a human STXBP1 protein), wherein the AAV capsid variant comprises an amino acid sequence encoded by the nucleotide sequence of any one of SEQ ID NOs: 12-35, or a nucleotide sequence at least 95% identical thereto.
   492. The AAV particle of 490 or 491, wherein the nucleotide sequence encoding the AAV capsid variant comprises the nucleotide sequence of any one of SEQ ID NOs: 12-35, or a nucleotide sequence at least 95% identical thereto.
   493. The AAV particle of any one of embodiments 1-299, 369-371, or 375-492, which has an increased tropism for a CNS cell or tissue, e.g., a brain cell, brain tissue, spinal cord cell, or spinal cord tissue, relative to the tropism of an AAV particle comprising a capsid comprising the amino acid sequence of SEQ ID NO: 138.
   494. The AAV particle of any one of embodiments 1-129, 168-299, 369-371, or 375-493, which transduces a brain region, e.g., a midbrain region (e.g., the hippocampus, or thalamus) or the brain stem, optionally wherein the level of transduction is at least 5, at least 10, at least 15, at least 20, at least 25, at least 30, at least 35, at least 40, at least 45, at least 50, at least 55, at least 60, or at least 65-fold greater as compared to an AAV particle comprising a capsid of SEQ ID NO: 138, e.g., when measured by an assay, e.g., an immunohistochemistry assay or a qPCR assay, e.g., as described in Example 2.
   495. The AAV particle of any one of embodiments 1-129, 168-299, 369-371, or 375-494, which transduces a brain region, e.g., a midbrain region (e.g., the hippocampus, or thalamus) or the brain stem, optionally wherein the level of transduction is at least 30, at least 35, at least 40, at least 45, at least 50, at least 55, at least 60, or at least 65-fold greater as compared to an AAV particle comprising a capsid of SEQ ID NO: 138, e.g., when measured by an assay, e.g., an immunohistochemistry assay or a qPCR assay, e.g., as described in Example 2.
   496. The AAV particle of any one of embodiments 1-129, 168-299, 369-371, or 375-495, which is enriched at least 3, at least 4, at least 5, at least 6, at least 7, at least 8, at least 9, or at least 10-fold, in the brain compared to an AAV particle comprising a capsid of SEQ ID NO: 138, e.g., when measured by an assay as described in Example 1.
   497. The AAV particle of any one of embodiments 1-129, 168-299, 369-371, or 375-4%, which is enriched at least 20, at least 25, at least 30, at least 35, at least 40, at least 45, at least 50, at least 55, at least 60, at least 65, at least 70, at least 75, at least 80 or at least 85-fold, in the brain compared to an AAV particle comprising a capsid of SEQ ID NO: 138, e.g., when measured by an assay as described in Example 1.
   498. The AAV particle of any one of embodiments 1-129, 168-299, 369-371, or 375-497, which is enriched in the brain of at least two to at least three species, e.g., a non-human primate and rodent (e.g., mouse), e.g., as compared to an AAV particle comprising a capsid of SEQ ID NO: 138.
   499. The AAV particle of any one of embodiments 1-129, 168-299, 369-371, or 375498, which is enriched at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 115, at least 120, at least 125, at least 130, at least 135, at least 140, at least 145, at least 150, at least 155, at least 160, at least 165, at least 170, at least 175, at least 180, at least 190, at least 200, at least 205, or at least 210-fold, in the brain of at least two to at least three species, e.g., a non-human primate and rodent (e.g., mouse), compared to an AAV particle comprising a capsid of SEQ ID NO: 138, e.g., when measured by an assay as described in Example 1 or 5.
   500. The AAV particle of embodiment 498 or 499, wherein the at least two to at least three species are Macaca fascicularis. Chlorocebus sabaeus, Callithrix jacchus, and/or mouse (e.g., BALB/c mice, C57B1/6 mice, and/or CD-1 outbred mice).
   501. The AAV particle of any one of embodiments 130-146, 369, 410414, 447454, 457, 458, 482, or 493, which is enriched at least 2, at least 2.5, at least 3, at least 3.5, at least 4, at least 4.5, at least 5, at least 5.5, at least 6, at least 6.5, at least 7, at least 7.5, or at least 8-fold, in the brain compared to an AAV particle comprising a capsid of SEQ ID NO: 981, e.g., when measured by an assay as described in Example 3.
   502. The AAV particle of any one of embodiments 147-167, 369, 410414, 447454, 457, 458, 482, or 493, which is enriched at least 2, at least 2.5, at least 3, at least 3.5, at least 4, at least 4.5, at least 5, or at least 5.5-fold, in the brain compared to an AAV particle comprising a capsid of SEQ ID NO: 982. e.g., when measured by an assay as described in Example 3.
   503. The AAV particle of any one of embodiments 1-129, 168-299, 369-371, or 375-500, which delivers an increased level of STXBP1 to a brain region, optionally wherein the level of the STXBP1 is increased by at least 10, at least 12, at least 15, at least 20, at least 25, at least 30, at least 35, at least 40, at least 45, at least 50, at least 55, at least 60, at least 65, or at least 70-fold, as compared to an AAV particle comprising a capsid of SEQ ID NO: 138, e.g., when measured by an assay, e.g., a qRT-PCR or a qPCR assay (e.g., as described in Example 2 or 8).
   504. The AAV particle of any one of embodiments 1-129, 168-299, 369-371, 375-500, or 503, which delivers an increased level of viral genomes to a brain region, optionally wherein the level of viral genomes is increased by at least 5, at least 10, at least 15, at least 17, at least 18, at least 19, at least 20, at least 25, at least 30, at least 35, at least 40, at least 45, or at least 50-fold, as compared to an AAV particle comprising a capsid of SEQ ID NO: 138, e.g., when measured by an assay, e.g., a qRT-PCR or a qPCR assay (e.g., as described in Example 2 or 8).
   505. The AAV particle of embodiment 503 or 504, wherein the brain region is a midbrain region (e.g., the hippocampus or thalamus), frontal cortex, temporal cortex, motor cortex, cerebral cortex, caudate, putamen, dentate nucleus, substantia nigra, or the brainstem.
   506. The AAV particle of any one of embodiments 1-129, 168-299, 369-371, 375-500, or 503-505, which is enriched at least 4, at least 5, at least 10, at least 15, at least 20, at least 25, at least 30, or at least 35-fold, in the spinal cord compared to an AAV particle comprising a capsid of SEQ ID NO: 138, e.g., when measured by an assay as described in Example 1 or 8, optionally wherein the region of the spinal cord is a thoracic spinal cord region, cervical spinal cord region. C5 ventral horn region, lumbar spinal cord region, or LS ventral horn region.
   507. The AAV particle of any one of embodiments 1-129, 168-299, 369-371, 375-500, or 503-506, which shows preferential transduction in a brain region relative to the transduction in the dorsal root ganglia (DRG).
   508. The AAV particle of any one of embodiments 1-129, 168-299, 369-371, 375-500, or 503-507, which shows preferential transduction in a brain region relative to the liver.
   509. The AAV particle of any one of embodiments 1-129, 168-299, 369-371, 375-500, or 503-508, which shows preferential transduction in a brain region relative to the transduction in the heart.
   510. The AAV particle of any one of embodiments 1-129, 168-299, 369-371, 375-500, or 503-509, which shows preferential transduction in a brain region relative to the transduction in the dorsal root ganglia (DRG) and the heart.
   511. The AAV particle of any one of the preceding embodiments, which is capable of transducing non-neural cells, e.g., glial cells (e.g., oligodendrocytes or astrocytes).
   512. The AAV particle of embodiment 511, wherein the non-neuronal cells comprise glial cells, oligodendrocytes (e.g., Olig2 positive oligodendrocytes), or astrocytes (e.g., Olig2 positive astrocytes).
   513. The AAV particle of any one of the preceding embodiments, which is capable of transducing Olig2 positive cells, e.g., Olig2 positive astrocytes or Olig2 positive oligodendrocytes.
   514. The AAV particle of any one of embodiments 369, 373, 447-454, 457, 458, or 482, which has increased tropism for a heart cell or tissue, e.g., a heart ventricle or heart atrium, relative to the tropism of an AAV particle comprising a capsid of SEQ ID NO: 138.
   515. The AAV particle of any one of embodiments 369, 373, 447454, 457, 458, 482, or 514, which is enriched at least 4, at least 5, at least 8, at least 10, at least 11, at least 12, at least 13, at least 14, at least 18, at least 19, at least 20, at least 21, at least 22, at least 24, at least 25, at least 27, at least 31, at least 33, or at least 34-fold, in the heart compared to an AAV particle comprising a capsid of SEQ ID NO: 138, e.g., when measured by an assay as described in Example 4.
   516. The AAV particle of any one of embodiments 369, 374, 447454, 457,458, 482, which has an increased tropism for a muscle cell or tissue (e.g., a quadriceps cell or a quadriceps tissue), relative to the tropism of an AAV particle comprising a capsid comprising the amino acid sequence of SEQ ID NO: 138.
   517. The AAV particle of any one of embodiments 369, 374, 447454, 457, 458, 482, which is enriched at least 4, at least 5, at least 8, at least 12, at least 17, at least 18, at least 20, at least 26, at least 27, at least 28, at least 30, or at least 36-fold, in the muscle compared to an AAV particle comprising a capsid of SEQ ID NO: 138, e.g., when measured by an assay as described in Example 4.
   518. The AAV particle of embodiment 516 or 517, wherein the muscle cell or tissue is a heart muscle (e.g., a heart ventricle or a heart atrium, or both), a quadriceps muscle, or both.
   519. The AAV particle of any one of the preceding embodiments, which is isolated and/or recombinant.
   [Embodiments 520-585 are intentionally absent.]
   586. The AAV particle of any one of the preceding embodiments, wherein the viral genome comprises a promoter operably linked to the STXBP1-encoding sequence.
   587. The AAV particle of embodiment 586, wherein the promoter is human elongation factor 1α-subunit (EF1α), cytomegalovirus (CMV) immediate-early enhancer and/or promoter, chicken β-actin (CBA) promoter, CAG promoter, CAG derivative promoter. β glucuronidase (GUSB) promoter, or ubiquitin C (UBC) promoter, neuron-specific enolase (NSE) promoter, platelet-derived growth factor (PDGF) promoter, platelet-derived growth factor B-chain (PDGF-β) promoter, intercellular adhesion molecule 2 (ICAM-2) promoter, synapsin (Syn) promoter, methyl-CpG binding protein 2 (MeCP2) promoter, Ca2+/calmodulin-dependent protein kinase 11 (CaMKII), metabotropic glutamate receptor 2 (mGluR2) promoter, neurofilament light (NFL) or heavy (NFH) promoter, β-globin minigene nβ2, preproenkephalin (PPE) promoter, enkephalin (Enk) and excitatory amino acid transporter 2 (EAAT2) promoter, glial fibrillary acidic protein (GFAP) promoter, myelin basic protein (MBP) promoter, a cardiovascular promoter (e.g., αMHC, cTnT, and CMV-MLC2k), a liver promoter (e.g., hAAT, TBG), a skeletal muscle promoter (e.g., desmin, MCK. C512) or a fragment, e.g., a truncation, or a functional variant thereof.
   [Embodiments 588 and 589 are intentionally absent.]
   590. The AAV particle of any one of embodiments 586-589, wherein the viral genome further comprises a polyA sequence.
   591. The AAV particle of any one of embodiments 586-590, wherein the viral genome further comprises an inverted terminal repeat (ITR) sequence.
   592. The AAV particle of any one of embodiments 586-591, wherein the viral genome comprises an ITR sequence positioned 5′ relative to the STXBP1-encoding sequence (e.g., encoding a human STXBP1 protein).
   593. The AAV particle of any one of embodiments 586-592, wherein the viral genome comprises an ITR sequence positioned 3′ relative to the STXBP1-encoding sequence (e.g., encoding a human STXBP1 protein).
   594. The AAV particle of any one of embodiments 586-593, wherein the viral genome comprises an ITR sequence positioned 5′ relative to the STXBP1-encoding sequence (e.g., encoding a human STXBP1 protein) and an ITR sequence positioned 3′ relative to the STXBP1-encoding sequence (e.g., encoding a human STXBP1 protein).
   595. The AAV particle of any one of embodiments 586-594, wherein the viral genome further comprises an enhancer, a Kozak sequence, an intron region, and/or an exon region.
   [Embodiments 596-615 are intentionally absent.]
   616. The AAV particle of any one of the embodiments 586-595, wherein the viral genome further comprises a nucleotide sequence encoding a Rep protein, e.g., a non-structural protein, wherein the Rep protein comprises a Rep78 protein, a Rep68 protein, a Rep52 protein, and/or a Rep40 protein (e.g., a Rep78 and a Rep52 protein).
   617. The AAV particle of embodiment 616, wherein the AAV particle further comprises a nucleotide sequence encoding a Rep protein, e.g., a non-structuml protein, wherein the Rep protein comprises a Rep78 protein, a Rep68 protein, a Rep52 protein, and/or a Rep40 protein (e.g., a Rep78 and a Rep52 protein).
   618. The AAV particle of embodiment 616 or 617, wherein the Rep78 protein, the Rep68 protein, the Rep52 protein, and/or the Rep40 protein are encoded by at least one Rep gene.
   619. The AAV particle of any one of embodiments 586-618, wherein the viral genome further comprises a nucleic acid sequence encoding the AAV capsid variant of the AAV particle of any one of embodiments 1-519, 566, or 574.
   620. The AAV particle of any one of embodiments 575-619, wherein the AAV particle is an isolated and/or recombinant AAV particle.
   [Embodiment 621 is intentionally absent.]
   622. A cell, e.g., a host cell, comprising the AAV particle of any one of the preceding embodiments.
   623. The cell of embodiment 622, wherein the cell is a mammalian cell or an insect cell.
   624. The cell of embodiment 622 or 623, wherein the cell is a cell of a brain region or a spinal cord region, optionally a cell of the brain stem, hippocampus, or thalamus.
   625. The cell of any one of embodiments 622-624, wherein the cell is a neuron, a sensory neuron, a motor neuron, an astrocyte, a glial cell, oligodendrocyte, or a muscle cell (e.g., a cell of the heart, diaphragm, or quadriceps).
   [Embodiment 626 is intentionally absent.]
   627. A method of making an adeno-associated virus (AAV) particle, comprising
(i) providing a host cell comprising the viral genome comprising a STXBP1-encoding sequence; and
(ii) incubating the host cell under conditions suitable to encapsulate the viral genome in the AAV capsid variant as described in any one of embodiments 1-620;
thereby making the AAV particle.
   628. The method of embodiment 627, further comprising, prior to step (i), introducing a first nucleic acid molecule comprising the viral genome into the host cell.
   629. The method of embodiment 628, wherein the host cell comprises a second nucleic acid encoding the capsid variant.
   630. The method of embodiment 629, wherein the second nucleic acid molecule is introduced into the host cell prior to, concurrently with, or after the first nucleic acid molecule.
   631. A pharmaceutical composition comprising the AAV particle of any one of embodiments 1-620, and a pharmaceutically acceptable excipient.
   632. A method of delivering a payload to a cell or tissue (e.g., a CNS cell or CNS tissue), comprising administering an effective amount of the pharmaceutical composition of embodiment 631 or the AAV particle of any one of embodiments 1-620.
   633. The method of embodiment 632, wherein the cell is a cell of a brain region or a spinal cord region, optionally a cell of the frontal cortex, sensory cortex, motor cortex, caudate, cerebellar cortex, cerebral cortex, brain stem, hippocampus, or thalamus.
   634. The method of embodiment 632 or 633, wherein the cell is a neuron, a sensory neuron, a motor neuron, an astrocyte, a glial cell, or an oligodendrocyte.
   [Embodiment 635 is intentionally absent.]
   636. The method of any one of embodiments 632-634, wherein the cell or tissue is within a subject.
   637. The method of embodiment 636, wherein the subject has, has been diagnosed with having, or is at risk of having a genetic disorder, e.g., a monogenic disorder or a polygenic disorder.
   638. The method of embodiment 636 or 637, wherein the subject has, has been diagnosed with having, or is at risk of having a neurological, e.g., a neurodegenerative, disorder.
   [Embodiment 639 is intentionally absent.]
   640. The method of embodiment 636 or 637, wherein the subject has, has been diagnosed with having, or is at risk of having, a muscular disorder or a neuromuscular disorder.
   641. A method of treating a subject having or diagnosed with having a genetic disorder, e.g., a monogenic disorder or a polygenic disorder, comprising administering to the subject an effective amount of the pharmaceutical composition of embodiment 631 or the AAV particle of any one of embodiments 1-620.
   642. A method of treating a subject having or diagnosed with having a neurological disorder, e.g., a neurodegenerative disorder, comprising administering to the subject an effective amount of the pharmaceutical composition of embodiment 631 or the AAV particle of any one of embodiments 1-620.
   643. A method of treating a subject having or diagnosed with having a muscular disorder or a neuromuscular disorder, comprising administering to the subject an effective amount of the pharmaceutical composition of embodiment 631 or the AAV particle of any one of embodiments 1-620.
   [Embodiment 644 is intentionally absent.]
   645. The method of any one of embodiments 637-643, wherein the genetic disorder, neurological disorder, neurodegenerative disorder, muscular disorder, or neuromuscular disorder is STXBP1 encephalopathy, epileptic encephalopathy, Ohtahara syndrome, developmental encephalopathy, West syndrome, early myoclonic epileptic encephalopathy. Lennox-Gaustaut syndrome, autism (e.g., autism with STXBP1 mutations and optionally further mutations). Dravet syndrome (not caused by mutations in SCN1A), or Rett syndrome phenotype (not caused by mutation of MECP2 or CDKL5).
   646. The method of any one of embodiments 641-645, wherein treating comprises prevention of progression of the disorder in the subject.
   647. The method of embodiment 636-646, wherein the subject is a human.
   648. The method of any one of embodiments 636-647, wherein the AAV particle is administered to the subject intravenously, via intra-cisterna magna injection (ICM), intracerebrally, intrathecally, intracerebroventricularly, via intraparenchymal administration, intraarterially, or intramuscularly.
   649. The method of any one of embodiments 636-648, wherein the AAV particle is administered to the subject via focused ultrasound (FUS), e.g., coupled with the intravenous administration of microbubbles (FUS-MB), or MRI-guided FUS coupled with intravenous administration.
   650. The method of any one of embodiments 636-649, wherein the AAV particle is administered to the subject intravenously.
   651. The method of any one of embodiments 636-650, wherein the AAV particle is administered to the subject via intra-cisterna magna injection (ICM).
   652. The method of any one of embodiments 636-651, wherein the AAV particle is administered to the subject intraarterially.
   [Embodiment 653 is intentionally absent.]
   654. The method of any one of embodiments 648-652, wherein administration of the AAV particle results in an increased presence, level, and/or activity of an STXBP1 gene, mRNA, protein, or a combination thereof.
   655. The pharmaceutical composition of embodiment 631 or the AAV particle of any one of embodiments 1-620 for use in a method of delivering an STXBP1-encoding sequence to a cell or tissue.
   656. The pharmaceutical composition of embodiment 631 or the AAV particle of any one of embodiments 1-620 for use in a method of treating a genetic disorder, a neurological disorder, a neurodegenerative disorder, a muscular disorder, or a neuromuscular disorder.
   657. The pharmaceutical composition of embodiment 631 or the AAV particle of any one of embodiments 1-620 for use in the manufacture of a medicament.
   658. Use of the pharmaceutical composition of embodiment 631 or the AAV particle of any one of embodiments 1-620 in the manufacture of a medicament.
   659. Use of the pharmaceutical composition of embodiment 631 or the AAV particle of any one of embodiments 1-620 in the manufacture of a medicament for treating a genetic disorder, a neurological disorder, or a neurodegenerative disorder, a muscular disorder, or a neuromuscular disorder.
   660. An AAV particle comprising the AAV capsid variant of any one of embodiments 1-519, e.g., any one of embodiments 507-510, and a viral genome comprising a nucleotide sequence that encodes a syntaxin-binding protein 1 (STXBP1) protein (i.e., an STXBP1-encoding sequence), wherein the nucleotide sequence is at least 90% (e.g., at least 90%, at least 91%, at least 92%, at least 93%, at least 94%, at least 95%, at least 96%, at least 97%, at least 98%, at least 99%, or 100%) identical to the nucleotide sequence of SEQ ID NO: 6414.
   661. The AAV particle of embodiment 660, wherein the nucleotide sequence encoding the STXBP1 protein (i.e., the STXBP1-encoding sequence) comprises a nucleotide sequence at least 90% identical to SEQ ID NO: 6414.
   662. The AAV particle of embodiment 660 or embodiment 661, wherein the nucleotide sequence encoding the STXBP1 protein (i.e., the STXBP1-encoding sequence) comprises a nucleotide sequence at least 95% identical to SEQ ID NO: 6414.
   663. The AAV particle of any one of embodiments 660-662, wherein the nucleotide sequence encoding the STXBP1 protein (i.e., the STXBP1-encoding sequence) comprises the nucleotide sequence of SEQ ID NO: 6414.
   [Embodiment 664 is intentionally absent.]
   665. The AAV particle of any one of embodiments 660-663, wherein the AAV capsid variant comprises (a) a VP1 protein comprising the amino acid sequence of SEQ ID NO: 982; (b) a VP2 protein comprising the amino acid sequence of positions 138-742 of SEQ ID NO: 982; or (c) a VP3 protein comprising the amino acid sequence of positions 203-742 of SEQ ID NO: 982; or wherein the AAV capsid variant is encoded by the nucleotide sequence of SEQ ID NO: 984 or a sequence at least 90% identical (e.g., at least 90%, at least 91%, at least 92%, at least 93%, at least 94%, at least 95%, at least 96%, at least 97%, at least 98%, or at least 99% identical) thereto.
   666. The AAV particle of any one of embodiments 660663, wherein the AAV capsid variant comprises no more than three amino acid substitutions relative to the amino acid sequence of HDSPHK (SEQ ID NO: 2), wherein the AAV capsid variant comprises an amino acid sequence that is at least 99% identical to SEQ ID NO: 982.
   [Embodiments 667-668 are intentionally absent.]
   669. The AAV particle of any one of embodiments 660-666, wherein the encoded STXBP1 protein comprises the amino acid sequence of SEQ ID NO: 6413, or an amino acid sequence at least 70% (e.g., at least 70%, at least 75%, at least 80%, at least 85%, at least 90%, at least 91%, at least 92%, at least 93%, at least 94%, at least 95%, at least 96%, at least 97%, at least 98%, or at least 99%) identical thereto.
   670. The AAV particle of embodiment 669, wherein the nucleotide sequence encoding the STXBP1 protein (i.e., the STXBP1-encoding sequence) comprises the nucleotide sequence of SEQ ID NO: 6414, or a nucleotide sequence at least 90% (e.g., at least 90%, at least 91%, at least 92%, at least 93%, at least 94%, at least 95%, at least 96%, at least 97%, at least 98%, or at least 99%) identical thereto.
   671. The AAV particle of embodiment 669 or 670, wherein the nucleotide sequence encoding the STXBP1 protein (i.e., the STXBP1-encoding sequence) comprises the nucleotide sequence of SEQ ID NO: 6414.
   672. The AAV particle of any one of embodiments 660-671, wherein the viral genome further comprises a nucleotide sequence encoding a signal sequence.
   [Embodiment 673 is intentionally absent].
   674. The AAV particle of embodiment 672, wherein the nucleotide sequence encoding the signal sequence is located 5′ relative to the STXBP1-encoding sequence.
   675. The AAV particle of embodiment 674, wherein the STXBP1-encoding sequence comprises the nucleotide sequence of SEQ ID NO: 6414 or a nucleotide sequcnce that is at least 97% (e.g., at least
   97%, at least 98%, or at least 99% identical) thereto. 676. The AAV particle of embodiment 675, wherein the STXBP1-encoding sequence comprises the nucleotide sequence of SEQ ID NO: 6414.
   677. The AAV particle of embodiment 675, wherein the STXBP1-encoding sequence consists of the nucleotide sequence of SEQ ID NO: 6414.
   678. The AAV particle of any one of embodiments 660-677, wherein the viral genome further comprises a promoter operably linked to the STXBP1-encoding sequence.
   679. The AAV particle of any one of embodiments 660-678, wherein the viral genome further comprises an enhancer.
   [Embodiments 680-681 are intentionally absent.]
   682. The AAV particle of embodiment 678 or 679, wherein the promoter comprises a tissue-specific promoter.
   683. The AAV particle of embodiments 678 or 679, wherein the promoter comprises a ubiquitous promoter.
   684. The AAV particle of any one of embodiments 678-683, wherein the promoter comprises: an EF-la promoter, a CB promoter, a chicken β-actin (CBA) promoter or its derivative, a CAG promoter or its derivative, a CMV immediate-early enhancer and/or promoter, a β glucuronidase (GUSB) promoter, a ubiquitin C (UBC) promoter, a neuron-specific enolase (NSE) promoter, a platelet-derived growth factor (PDGF) promoter, a platelet-derived growth factor B-chain (PDGF-β) promoter, an intercellular adhesion molecule 2 (ICAM-2) promoter, a synapsin (Syn) promoter, a synapsin 1 promoter (Syn1), a methyl-CpG binding protein 2 (MeCP2) promoter, a Ca2+/calmodulin-dependent protein kinase I (CaMKII) promoter, a metabotropic glutamate receptor 2 (mGluR2) promoter, a neurofilament light (NFL) or heavy (NFH) promoter, a β-globin minigene nP2 promoter, a preproenkephalin (PPE) promoter, an enkephalin (Enk) and excitatory amino acid transporter 2 (EAAT2) promoter, a glial fibrillary acidic protein (GFAP) promoter, a myelin basic protein (MBP) promoter, a cardiovascular promoter (e.g., aMHC, cTnT, and CMV-MLC2k), a liver promoter (e.g., hAAT. TBG), a skeletal muscle promoter (e.g., desmin, MCK. C512) or a fragment, e.g., a truncation, or a functional variant thereof.
   [Embodiments 685-712 are intentionally absent.]
   713. The AAV particle of any one of embodiments 660-684, wherein the viral genome further comprises an inverted terminal repeat (ITR) sequence.
   714. The AAV particle of embodiment 713, wherein the ITR sequence is positioned 5′ relative to the STXBP1-encoding sequence.
   715. The AAV particle of embodiment 713 or embodiment 714, wherein the ITR sequence is positioned 3′ relative to the STXBP1-encoding sequence.
   716. The AAV particle of any one of embodiments 713-715, wherein the viral genome comprises an ITR positioned 5′ relative to the STXBP1-encoding sequence and an ITR positioned 3′ relative to the STXBP1-encoding sequence.
   722. The AAV particle of any one of embodiments 660-721, wherein the viral genome further comprises a polyadenylation (polyA) region.
   724. The AAV particle of any one of embodiments 660-723, wherein the viral genome further comprises an intron.
   727. The AAV particle of any one of embodiments 660-726, wherein the viral genome further comprises an exon, e.g., at least one, at least two, or at least three exons.
   728. The AAV particle of any one of embodiments 660-727, wherein the viral genome further comprises a Kozak sequence.
   [Embodiments 729-761 are intentionally absent.]
   762. The AAV particle of any one of embodiments 660-728, further comprising a nucleic acid encoding a Rep protein, wherein the Rep protein comprises a Rep78 protein, a Rep68 protein, a Rep52 protein, and/or a Rep40 protein.
   763. The AAV particle of embodiment 762, wherein the Rep78 protein, the Rep68 protein, the Rep52 protein, and/or the Rep40 protein are encoded by at least one Rep gene.
   764. A vector encoding the AAV particle of any one of embodiments 660-763.
   765. A cell comprising the AAV particle of any one of embodiments 660-763 or the vector of embodiment 764.
   766. The cell of embodiment 765, which is a mammalian cell, e.g., an HEK293 cell, an insect cell, e.g., an Sf9 cell, or a bacterial cell.
   767. A method of making a recombinant adeno-associated virus (AAV) particle, the method comprising
(i) providing a host cell comprising a viral genome comprising the nucleotide sequence of SEQ ID NO: 6414, or a sequence at least 90% (e.g., at least 91% 92%, 93%, 94%, 95%, 96%, 97%, 98%, or 99%) identical thereto; and
(ii) incubating the host cell under conditions suitable to enclose the viral genome in a capsid variant comprising the amino acid sequence of SEQ ID NO: 982;
thereby making the recombinant AAV particle.
   768. The method of embodiment 767, further comprising, prior to step (i), introducing a first nucleic acid molecule comprising the viral genome into the host cell.
   769. The method of embodiment 767 or embodiment 768, wherein the host cell comprises a second nucleic acid encoding the capsid variant.
   770. The method of embodiment 769, further comprising introducing the second nucleic acid into the cell.
   771. The method of embodiment 769 or embodiment 770, wherein the second nucleic acid molecule is introduced into the host cell prior to, concurrently with, or after the first nucleic acid molecule.
   772. The method of any one of embodiments 767-772, wherein the host cell comprises a mammalian cell, e.g., an HEK293 cell, an insect cell, e.g., an Sf9 cell, or a bacterial cell.
   773. A pharmaceutical composition comprising the AAV particle of any one of embodiments 660-763 and a pharmaceutically acceptable excipient.
   774. A method of delivering a STXBP1 protein to a subject, comprising administering to the subject an effective amount of the pharmaceutical composition of embodiment 773 or the AAV particle of any one of embodiments 660-763, thereby delivering the nucleic acid encoding a STXBP1 protein (i.e., the STXBP1-encoding sequence) to the subject.
   775. The method of embodiment 774, wherein the subject has, has been diagnosed with having, or is at risk of having an STXBP1-related disorder.
   776. The method of embodiment 774 or embodiment 775, wherein the subject has, has been diagnosed with having, or is at risk of having a neurodegenerative or neuromuscular disorder.
   777. A method of treating a subject having or diagnosed with having an STXBP1-related disorder, comprising administering to the subject an effective amount of the pharmaceutical composition of embodiment 773 or the AAV particle of any one of embodiments 660-763, thereby treating the STXBP1-related disorder in the subject.
   778. A method of treating a subject having or diagnosed with having a neurodegenerative or neuromuscular disorder, comprising administering to the subject an effective amount of the pharmaceutical composition of embodiment 773 or the AAV particle of any one of embodiments 660-763, thereby treating the neurodegenerative or neuromuscular disorder in the subject.
   779. The method of any one of embodiments 774-778, wherein the STXBP1-related disorder or the neurodegenerative or neuromuscular disorder comprises STXBP1 encephalopathy, epileptic encephalopathy, Ohtahara syndrome, developmental encephalopathy. West syndrome, early myoclonic epileptic encephalopathy, Lennox-Gaustaut syndrome, autism (e.g., autism with STXBP1 mutations and optionally further mutations). Dravet syndrome (not caused by mutations in SCN1A), and Rett syndrome phenotype (not caused by mutation of MECP2 or CDKL5).
   780. A method of treating a subject having or diagnosed with having STXBP1 encephalopathy, comprising administering an effective amount of the pharmaceutical composition of embodiment 773 or the AAV particle of any one of embodiments 660-763, thereby treating STXBP1 encephalopathy in the subject.
   781. The method of embodiment 779 or embodiment 780, wherein the subject has one or more mutations in STXBP1.
   784. A method of treating a subject having or diagnosed with having STXBP1 encephalopathy, comprising administering an effective amount of the pharmaceutical composition of embodiment 773 or the AAV particle of any one of embodiments 660-763, thereby treating STXBP1 encephalopathy in the subject.
   [Embodiments 785-788 are intentionally absent.]
   789. The method of any one of embodiments 774-784, wherein the subject has a reduced level of STXBP1 activity as compared to a reference level.
   790. The method of embodiment 789, wherein the reference level comprises the level of STXBP1 activity in a subject that does not have an STXBP1-related disorder, a neuromuscular disorder, and/or a neurodegenerative disorder.
   791. The method of any one of embodiments 777-790, wherein treating results in amelioration of at least one symptom of the STXBP1-related disorder, the neurodegenerative disorder, and/or the neuromuscular disorder in the subject.
   792. The method of embodiment 791, wherein the at least one symptom of the STXBP1-related disorder, the neurodegenerative disorder, and/or the neuromuscular disorder comprises reduced STXBP1 activity, accumulation of glucocerebroside and other glycolipids, e.g., within immune cells (e.g., macrophages), build-up of synuclein aggregates (e.g., Lewy bodies), developmental delay, progressive encephalopathy, progressive dementia, ataxia, myoclonus, oculomotor dysfunction, bulbar palsy, generalized weakness, trembling of a limb, depression, visual hallucinations, cognitive decline, dystonia, or a combination thereof.
   793. The method of any one of embodiments 774-792, wherein the subject is a human.
   794. The method of any one of embodiments 774-793, wherein the subject is a juvenile, e.g., between 6 years of age to 20 years of age.
   795. The method of any one of embodiments 774-793, wherein the subject is an adult, e.g., above 20 years of age.
   796. The method of any one of embodiments 774-795, wherein the subject has one or more mutations in a STXBP1 gene, STXBP1 mRNA, and/or STXBP1 protein.
   797. The method of any one of embodiments 774-79%, wherein the AAV particle is administered to the subject intravenously, intracerebrally, via intrathalamic (ITH) administration, intramuscularly, intrathecally, intracerebroventricularly, via intraparenchymal administration, via focused ultrasound (FUS), e.g., coupled with the intravenous administration of microbubbles (FUS-MB), or MRI-guided FUS coupled with intravenous administration, or via intra-cistema magna injection (ICM).
   798. The method of any one of embodiments 774-796, wherein the AAV particle is administered intravenously.
   799. The method of any one of embodiments 774-7%, wherein the AAV particle is administered via intravenous injection, optionally wherein the intravenous injection is via focused ultrasound (FUS), e.g., coupled with the intravenous administration of microbubbles (FUS-MB), or MRI-guided FUS coupled with intravenous administration.
   800. The method of any one of embodiments 774-799, wherein the AAV particle is administered to a cell, tissue, or region of the CNS, e.g., a region of the brain or spinal cord, e.g., the parenchyma, the cortex, substantia nigra, caudate cerebellum, striatum, corpus callosum, cerebellum, brain stem caudate-putamen, thalamus, superior colliculus, the spinal cord, or a combination thereof.
   801. The method of any one of embodiments 774-800, wherein the AAV particle is administered to at least two tissues, or regions of the CNS, e.g., bilateral administration.
   802. The method of any one of embodiments 774-799, wherein the AAV particle is administered to the cerebral spinal fluid, the serum, or a combination thereof.
   803. The method of any one of embodiments 774-802, further comprising performing a blood test, performing an imaging test, collecting a CNS biopsy sample, collecting a tissue biopsy. (e.g., a biopsy of the lung, liver, or spleen), collecting a blood or serum sample, or collecting an aqueous cerebral spinal fluid biopsy.
   804. The method of any one of embodiments 774-803, which further comprises evaluating, e.g., measuring, the level of STXBP1 expression, e.g., STXBP1 gene. STXBP1 mRNA, and/or STXBP1 protein expression, in the subject, e.g., in a cell, tissue, or fluid, of the subject, optionally wherein the level of STXBP1 protein is measured by an assay described herein, e.g., an ELISA, a Western blot, or an immunohistochemistry assay.
   805. The method of embodiment 804, wherein measuring the level of STXBP1 expression is performed prior to, during, or subsequent to treatment with the AAV particle.
   806. The method of embodiment 804 or embodiment 805, wherein the cell or tissue is a cell or tissue of the central nervous system (e.g., parenchyma) or a peripheral cell or tissue (e.g., the liver, heart, and/or spleen).
   807. The method of any one of embodiments 774-806, wherein the administration results in increased level of STXBP1 protein expression in a cell or tissue of the subject, relative to reference level, e.g., a subject that has not received treatment, e.g., has not been administered the AAV particle.
   808. The method of any one of embodiments 774-807, which further comprises evaluating, e.g., measuring, the level of STXBP1 activity in the subject, e.g., in a cell or tissue of the subject, optionally wherein the level of STXBP1 activity is measured by an assay described herein.
   809. The method of any one of embodiments 774-808, wherein the administration results in an increase in at least one, at least two, or all of:
(i) the level of STXBP1 activity in a cell, tissue. (e.g., a cell or tissue of the CNS, e.g., the cortex, striatum, thalamus, cerebellum, and/or brainstem), and/or fluid (e.g., CSF and/or serum), of the subject, as compared to a reference level, e.g., a subject that has not received treatment, e.g., has not been administered the AAV particle;
(ii) the level of viral genomes (VG) per cell in a CNS tissue (e.g., the cortex, striatum, thalamus, cerebellum, brainstem, and/or spinal cord) of the subject, as compared to a peripheral tissue, wherein the level of VGs per cell is at least 4 to at least 10 fold lower than the levels in the CNS tissue, e.g., as measured by an assay as described herein; and/or
(iii) the level of STXBP1 mRNA expression in a cell or tissue (e.g., a cell or tissue of the CNS, e.g., the cortex, thalamus, and/or brainstem) as compared to a reference level, e.g., a subject that has not received treatment (e.g., has not been administered the AAV particle), or endogenous STXBP1 mRNA levels, e.g., as measured by an assay as described herein.
   810. The method of any one of embodiments 774-809, wherein further comprising administration of an additional therapeutic agent and/or therapy suitable for treatment or prevention of the disease associated STXBP1 expression, the neurodegenerative disorder, and/or the neuromuscular disorder.
   811. The method of embodiment 810, wherein the additional therapeutic agent and/or therapy comprises an anti-epileptic drug (e.g., levetiracetam, phenobarbital, clobazam, topiramate), adrenocorticotropic hormone, or a combination thereof.
   812. The pharmaceutical composition of embodiment 773 or the AAV particle of any one of embodiments 660-763 for use in the treatment of an STXBP1-related disorder, a neuromuscular disorder, and/or a neurodegenerative disorder.
   813. Use of an effective amount of the pharmaceutical composition of embodiment 773 or the AAV particle of any one of embodiments 660-763 in the manufacture of a medicament for the treatment of an STXBP1-related disorder, a neuromuscular disorder, and/or a neurodegenerative disorder.

The details of various aspects or embodiments of the present disclosure are set forth below. Other features, objects, and advantages of the disclosure will be apparent from the description and the claims. In the description, the singular forms also include the plural unless the context clearly dictates otherwise. Unless defined otherwise, all technical and scientific terms used herein have the same meaning as commonly understood by one of ordinary skill in the art in the field of this disclosure. In the case of conflict, the present description will control.

### BRIEF DESCRIPTION OF THE DRAWINGS

FIG. 1 depicts biodistribution (VG/cell) in the motor cortex, frontal cortex, putamen, substantia nigra, dentate nucleus, cervical spinal cord ventral horn, DRG, liver, and heart in cynomolgus monkeys at 28 days post-IV injection of TTM-002, GBA_VG17-HA, AAV9.GBA_VG17-HA, or vehicle control.

FIG. 2 depicts mRNA expression of the GBA1 transgene in the motor cortex, frontal cortex, putamen, substantia nigra, dentate nucleus, cervical spinal cord ventral horn, DRG, liver, and heart in cynomolgus monkeys at 28 days post-IV injection of TTM-02.GBA_VG17-HA, AAV9.GBA_VG17-HA, or vehicle control.

FIG. 3A is a graph showing the percentage of HA positive cells (percent of cells transduced by the indicated capsid variant) in the cortex in mice on the Y axis at the indicated doses on the X-axis (from highest dose to lowest dose: 1e14 vg/kg. 3.2e13 vg/kg, 1e13 vg/kg. 3.2e12 vg/kg, or 1e12 vg/kg) at 28 days post-intravenous administration of AAV particles comprising the TTM-002 or TTM-027 AAV capsid variant. FIG. 3B is a graph showing the mRNA transgene expression relative to the housekeeping gene in the brains of the mice on the Y axis at the indicated doses on the X-axis (from highest to lowest dose: 1e14 vg/kg. 3.2e13 vg/kg, 1e13 vg/kg, 3.2e12 vg/kg, or 1e12 vg/kg) at 28 days post-intravenous administration of AAV particles comprising the TTM-002 or TTM-027 AAV capsid variant.

FIG. 4A is a graph showing the percentage of transduced cells having HA+ nuclei as measured by co-localization of nuclear H2B-HA staining and hematoxylin (% HA+ cells) in the indicated brain regions (temporal cortex, caudate, thalamus, or hippocampus) of African green monkeys. Measurements are at day 28 post-intravenous injection of AAV particles comprising the TTM-002 capsid variant or the AAV9 capsid control and a self-complementary genome encoding a histone 2B protein with an HA-tag at a dose of Ic13 VG/kg. FIG. 4B is a graph showing the percentage of HA+ cells among cells positive for the indicated marker (NeuN+ neurons, SM311+ Neurons, GFAP+ astrocytes, or Sox9+ astrocytes) in the indicated brain regions (temporal cortex, caudate, thalamus, or hippocampus) of African green monkeys. Measurements are at day 28 post-intravenous injection of AAV particles comprising the TTM-002 capsid variant and a self-complementary genome encoding a histone 2B protein with an HA-tag at a dose of 1e13 VG/kg. Plotted data in FIGS. 4A-4B represent one slice per monkey (n=2), Quantitative image analysis was performed on 1e3 to 1e5 cells according to region size. All P values are derived from an unpaired two-tailed t-test.

FIGS. 5A-5D are a series of graphs showing tropism of TTM-0 and TTM-02 relative to the AAV9 control in the brain and liver at 28 days post-intravenous administration in mice at a dose of 1e13 VG/kg. FIG. 5A shows the viral genomes (VG)/diploid genomes (DG) in the brain for the AAV9 control, TTM-001, or TTM-002; FIG. 5B shows brain RNA (fold vs AAV9) for the AAV9 control, TTM-001, or TTM-002; FIG. 5C shows the VG/DG in the liver for the AAV9 control, TTM-001, or TTM-02; and FIG. 5D shows the liver RNA (fold vs AAV9) for the AAV9 control, TTM-001, or TTM-02. Each data point represents an individual mouse and all plotted values represent mean±SD (n=3). P values are derived from an unpaired two-tailed t-test.

### DETAILED DESCRIPTION

### Overview

Described herein, inter alia, are compositions comprising an AAV capsid variant comprising a sequence encoding a STXBP1 protein, e.g., a wildtype STXBP1 protein, e.g., a wilditype human STXBP1 protein. In some embodiments, the present disclosure provides a method for delivering the AAV capsid variant comprising the sequence encoding the STXBP1 protein to a cell or tissue in a subject. In some embodiments, the present disclosure provides a method for delivering the AAV capsid variant, thereby providing a STXBP1 protein, e.g., wildtype STXBP1 protein, e.g., a wildtype human STXBP1 protein, to a cell or tissue in a subject. In some embodiments, the AAV capsid variants described herein have enhanced CNS tropism compared to other cells or tissues in the body, e.g., liver and/or the DRG.

AAVs have proven to be useful as a biological tool due to their relatively simple structure, their ability to infect a wide range of cells (including quiescent and dividing cells) without integration into the host genome and without replicating, and their relatively benign immunogenic profile. Engineered adeno-associated vims (AAV) capsids with improved brain tropism represent an attractive solution to the limitations of CNS delivery. AAV-derived vectors are promising tools for clinical gene transfer because of their non-pathogenic nature, their low immunogenic profile, low rate of integration into the host genome, and long-term transgene expression in non-dividing cells. However, the transduction efficiency of naturally occurring AAVs in certain organs is too low for clinical applications, and capsid neutralization by pre-existing neutralizing antibodies may prevent treatment of a large proportion of patients. For these reasons, considerable efforts have been devoted to obtaining capsid variants with enhanced properties. Of many approaches tested so far, significant advances have resulted from directed evolution of AAV capsids using in vitro or in vivo selection of capsid variants created by capsid sequence randomization using either error-prone PCR, shuffling of various parent serotypes, or insertion of fully randomized short peptides at defined positions.

The genome of the virus may be modified to contain a minimum of components for the assembly of a functional recombinant virus, or viral particle, which is loaded with or engineered to target a particular tissue and express or deliver STXBP1. The genome of the virus may encode a STXBP1 protein, and the viral particle comprising said genome may be delivered to a target cell, tissue, or organism. In some embodiments, the genome encodes a human STXBP1 protein, e.g., a wildtype STXBP1 protein. In some embodiments, the target cell is a CNS cell. In some embodiments, the target tissue is a CNS tissue. In some embodiments, the target CNS tissue is brain tissue.

In some embodiments, the genome encodes a wildtype STXBP1 protein. In some embodiments, the genome comprises a codon-optimized, CpG-reduced (e.g., CpG-depleted) nucleotide sequence encoding a wildtype STXBP1 protein, e.g., as compared to a wildtype STXBP1 encoding sequence. In some embodiments, the target cell is a CNS cell. In some embodiments, the target tissue is a CNS tissue. The target CNS tissue may be brain tissue. In some embodiments, the brain target comprises caudate, Putamen, thalamus, superior colliculus, cortex, and corpus collosum.

Gene therapy presents an alternative approach for treating a STXBP1-related disorder such as a STXBP1-related neurodegenerative or neuromuscular disorder (e.g., STXBP1 encephalopathy, epileptic encephalopathy, Ohtahara syndrome, developmental encephalopathy, West syndrome, early myoclonic epileptic encephalopathy, Lennox-Gaustaut syndrome, autism (e.g., autism with STXBP1 mutations and optionally further mutations). Dravet syndrome (not caused by mutations in SCN1A), or Rett syndrome phenotype (not caused by mutation of MECP2 or CDKL5), AAVs are commonly used in gene therapy approaches as a result of a number of advantageous features. Without being bound by theory, it is believed in some embodiments, an AAV particle described herein, can be used to administer and/or deliver a gene encoding STXBP1 protein (e.g., human STXBP1 protein), preferentially to the CNS. In some embodiments, an AAV particle described herein can be used to administer and/or deliver a gene encoding STXBP1 protein (e.g., human STXBP1 protein) preferentially to the brain.

Provided herein are compositions and methods which may provide for improved features compared to prior AAV-mediated enzyme replacement approaches, including (i) increased biodistribution throughout the CNS (e.g., the cortex, striatum, thalamus, cerebellum, brainstem, and/or spinal cord), and the periphery, and/or (iii) elevated payload expression, e.g., STXBP1 mRNA expression, in multiple brain regions (e.g., cortex, thalamus, and brain stem) and the periphery; and (ii) increased STXBP1 activity in a cell, tissue. (e.g., a cell or tissue of the CNS, e.g., the cortex, striatum, thalamus, cerebellum, and/or brainstem), and/or fluid (e.g., CSF and/or serum), of the subject. In some embodiments, an AAV viral genome comprising a codon-optimized, CpG-reduced (e.g., CpG-depleted) nucleotide sequence encoding a STXBP1 protein (e.g., SEQ ID NO: 6414) results in high biodistribution in the CNS; increased STXBP1 activity in the CNS, peripheral tissues, and/or fluid; and successful transgene transcription and expression.

Also provided herein, are compositions comprising an AAV capsid variant, e.g., an AAV capsid variant described herein for delivery, e.g., vectorized delivery, of a nucleic acid encoding a STXBP1 protein, and methods of making and using the same. As demonstrated in the Examples below, certain AAV capsid variants described herein show multiple advantages over wild-type AAV9, including (i) increased penetrance through the blood brain barrier following intravenous administration, (ii) wider distribution throughout the multiple brain regions, e.g., frontal cortex, sensory cortex, motor cortex, putamen, thalamus, cerebellar cortex, dentate nucleus, caudate, and/or hippocampus, and/or (iii) elevated payload expression in multiple brain regions. Without being bound by theory, these advantages may be due, in part, to the dissemination of the AAV capsid variants through the brain vasculature. In sonic embodiments, the AAV capsids described herein enhance the delivery of a payload to multiple regions of the brain including for example, the frontal cortex, sensory cortex, motor cortex, putamen, thalamus, cerebellar cortex, dentate nucleus, caudate, and/or hippocampus.

Thus, the compositions and methods described herein can be used in the treatment of a STXBP1-related disorder, such as a STXBP1-related neurodegenerative or neuromuscular disorder (e.g., STXBP1 encephalopathy, epileptic encephalopathy, Ohtahara syndrome, developmental encephalopathy, West syndrome, early myoclonic epileptic encephalopathy. Lennox-Gaustaut syndrome, autism (e.g., autism with STXBP1 mutations and optionally further mutations). Dravet syndrome (not caused by mutations in SCN1A), or Rett syndrome phenotype (not caused by mutation of MECP2 or CDKL5). In some embodiments, the disclosure provides an AAV particle comprising one of the AAV capsid variants disclosed herein and an AAV viral genome comprising a nucleotide sequence encoding a STXBP1 protein for use in treating a STXBP1-related disorder, such as a STXBP1-related neurodegenerative or neuromuscular disorder (e.g., STXBP1 encephalopathy, epileptic encephalopathy, Ohtahara syndrome, developmental encephalopathy, West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5)).

### I. Compositions

Adeno-associated viral (AAV) Particles

AAVs have a genome of about 5,000 nucleotides in length and contain two open reading frames encoding the proteins responsible for replication (Rep) and the structural protein of the capsid (Cap). The open reading frames are flanked by two Inverted Terminal Repeat (ITR) sequences, which serve as the origin of replication of the viral genome. The wild-type AAV viral genome comprises nucleotide sequences for two open reading frames, one for the four non-structural Rep proteins (Rep78, Rep68, Rep52. Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736. VP2 refers to amino acids 138-736, and VP3 refers to amino acids 203-736. In some embodiments, with reference to the amino acid sequence of SEQ ID NO: 982, 36, or 4, VP1 comprises amino acids 1-742, VP2 comprises amino acids 138-742, and VP3 comprises amino acids 203-742. In other words, VP1 is the full-length capsid protein sequence, while VP2 and VP3 are shorter components of the whole. As a result, changes in the sequence in the VP3 region are also changes to VP1 and VP2; however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid. Without being bound by theory, the AAV capsid typically comprises a molar ratio of 1:1:10 of VP1:VP2:VP3.

The AAV particle typically requires a co-helper (e.g., adenovirus) to undergo productive infection in cells. In the absence of such helper functions, the AAV virions essentially enter host cells but do not integrate into the cells' genome.

AAV particles have been investigated for delivery of gene therapeutics because of several unique features. Non-limiting examples of the features include (i) the ability to infect both dividing and non-dividing cells; (ii) a broad host range for infectivity, including human cells; (iii) wild-type AAV has not been associated with any disease and has not been shown to replicate in infected cells; (iv) the lack of cell-mediated immune response against the particle, and (v) the non-integrative nature in a host chromosome thereby reducing potential for long-term genetic alterations. Moreover, infection with AAV particles has minimal influence on changing the pattern of cellular gene expression (Stilwell and Samulski et al., Biotechniques, 2003, 34, 148, the contents of which are herein incorporated by reference in their entirety).

Typically. AAV particles for STXBP1 delivery may be recombinant viral particles which are replication defective as they lack sequences encoding functional Rep and Cap proteins within the viral genome. In some cases, the replication defective AAV particles may lack most or all coding sequences and essentially only contain one or two AAV ITR sequences and a nucleic acid sequence encoding a STXBP1 protein (e.g., human STXBP1 protein).

In some embodiments, the AAV particles of the present disclosure may be introduced into mammalian cells.

AAV particles may be modified to enhance the efficiency of delivery. Such modified AAV particles of the present disclosure can be packaged efficiently and can be used to successfully infect the target cells at high frequency and with minimal toxicity.

In other embodiments, AAV particles of the present disclosure may be used to deliver STXBP1 to the central nervous system (see, e.g., U.S. Pat. No. 6,180,613; the contents of which are herein incorporated by reference in their entirety) or to specific tissues of the CNS.

It is understood that the compositions described herein may have additional conservative or non-essential amino acid substitutions, which do not have a substantial effect on their functions.

In some embodiments, an AAV capsid variant disclosed herein comprises a modification in loop IV of AAV9, e.g., at positions between 449-460, e.g., at position 454 and/or 456, numbered relative to SEQ ID NO: 4, 36, 138, 981, or 982. In some embodiments, loop (e.g., loop IV) is used interchangeably herein with the term variable region (e.g., variable region IV), or VR (e.g., VR-IV). In some embodiments loop IV comprises positions 449-475 (e.g., amino acids KTINGSGQNQQTLKFSVAGPSNMAVQG (SEQ ID NO: 6404)), numbered according to SEQ ID NO: 138. In some embodiments loop IV comprises positions 449-460 (e.g., amino acids KTINGSGQNQQT (SEQ ID NO: 6405)), numbered according to SEQ ID NO: 138. In some embodiments, loop IV or variable region IV (VR-IV) is as described in DiMattia et al. “Structural Insights into the Unique Properties of the Adeno-Associated Virus Serotype 9.” Journal of Virology, 12(86):6947-6958 (the contents of which are hereby incorporated by reference in their entirety), e.g., comprising positions 452-460 (e.g., NGSGQNQQT (SEQ ID NO: 4487)), numbered according to SEQ ID NO: 138.

The AAV particles and payloads of the disclosure may be delivered to one or more target cells, tissues, organs, or organisms. In some embodiments, the AAV particles demonstrate enhanced tropism for a target cell type, tissue or organ. As a non-limiting example, the AAV particle may have enhanced tropism for cells and tissues of the central or peripheral nervous systems (CNS and PNS, respectively). In some embodiments, an AAV particle may, in addition, or alternatively, have decreased tropism for a cell-type, tissue or organ.

In some embodiments, AAV particles are used as a biological tool due to a relatively simple structure, their ability to infect a wide range of cells (including quiescent and dividing cells) without integration into the host genome and without replicating, and their relatively benign immunogenic profile. The gnome of the virus may be manipulated to contain a minimum of components for the assembly of a functional recombinant virus, or viral particle, which is loaded with or engineered to target a particular tissue and express or deliver a desired payload.

In some embodiments, the AAV particle is a recombinant AAV particle. In some embodiments, the wild-type AAV viral genome is a linear, single-stranded DNA (ssDNA) molecule approximately 5,000 nucleotides (nt) in length. In some embodiments, inverted terminal repeats (ITRs) cap the viral genome at both the 5′ and the 3′ end, providing origins of replication for the viral genome. In some embodiments, an AAV viral genome comprises two ITR sequences. In some embodiments, the ITRs have a characteristic T-shaped hairpin structure defined by a self-complementary region (145nt in wild-type AAV) at the 5′ and 3′ ends of the ssDNA which form an energetically stable double stranded region. In some embodiments, the double stranded hairpin structures comprise multiple functions including, but not limited to, acting as an origin for DNA replication by functioning as primers for the endogenous DNA polymerase complex of the host viral replication cell.

In some embodiments, the wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2. VP3, encoded by capsid genes or Cap genes). The Rep proteins are used for replication and packaging, while the capsid proteins are assembled to create the protein shell of the AAV, or AAV capsid polypeptide, e.g., an AAV capsid variant. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. In some embodiments, for any one of the amino acid sequences of SEQ ID NO: 981 or 982. VP1 comprises amino acids 1-742, VP2 comprises amino acids 138-742, and VP3 comprises amino acids 203-742. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3.

AAV particles of the present disclosure may be produced recombinantly and may be based on AAV reference sequences. In addition to single-stranded AAV viral genomes (e.g., ssAAVs), the present disclosure also provides for self-complementary AAV (scAAVs) viral genomes, scAAV viral genomes contain DNA strands that anneal together to form double-stranded DNA. By skipping second strand synthesis, scAAVs allow for rapid expression in the transduced cell. In some embodiments, the AAV particle of the present disclosure is an scAAV. In sonic embodiments, the AAV particle of the present disclosure is an ssAAV.

Methods for producing and/or modifying AAV particles are disclosed in the art such as pseudotyped AAV particles (PCT Patent Publication Nos. WO200028004; WO200123001; WO2004112727; WO2005005610; and WO2005072364, the content of each of which is incorporated herein by reference in its entirety).

As described herein, the AAV particles of the disclosure comprising an AAV capsid variant, and a viral genome, have enhanced tropism for a cell-type or a tissue, e.g., a CNS cell-type, region, or tissue.

### AAV Capsid Variants

Disclosed herein are AAV particles comprising an AAV capsid variant comprising one or more modifications (e.g., comprising one or more insertions and/or substitutions relative to a wildtype AAV capsid) for enhanced or improved transduction of a target tissue (e.g., cells, regions, and/or tissues of the CNS and/or PNS). In some embodiments, the one or more modifications comprises a peptide insertion relative to a wildtype AAV capsid. In some embodiments, the one or more modifications is present in a capsid protein of the AAV particle. In some embodiments, the one or more modifications is present in a VP1, VP2, and/or VP3 protein of the AAV particle.

In some embodiments, the one or more modifications (e.g., the peptide insertion and/or amino acid substitution relative to a wildtype AAV capsid) is in loop IV of the AAV capsid variant. In some embodiments, the AAV capsid variant is an AAV9 capsid variant

In some embodiments, the variant is an insertional variant. As used herein, the term “insertional variant” refers to a polypeptide comprising one or more amino acids inserted, e.g., “immediately adjacent” or “immediately subsequent” to a position in a reference amino acid sequence. “Immediately adjacent” or “immediately subsequent” to an amino acid refers to the insertion sequence being connected to either the alpha-carboxy or alpha-amino functional group of the amino acid. In some embodiments, the variant is a deletion variant. As used herein, the term “deletion variant” refers to a polypeptide comprising one or more amino acids removed from a reference amino acid sequence. In some embodiments, the variant is a substitution variant. As used herein, the term “substitution variant” refers to a polypeptide comprising one or more amino acid changes from a reference amino acid sequence. In some embodiments, the variant is an insertional variant and a substitution variant.

In some embodiments, the one or more modifications in the AAV capsid may increase distribution of an AAV particle to a cell, region, or tissue of the CNS. The cell of the CNS may be, but is not limited to, neurons (e.g., excitatory, inhibitory, motor, sensory, autonomic, sympathetic, parasympathetic. Purkinje, Betz, etc.), glial cells (e.g., microglia, astrocytes, oligodendrocytes) and/or supporting cells of the brain such as immune cells (e.g., T cells). The tissue of the CNS may be, but is not limited to, the cortex (e.g., frontal, parietal, occipital, and/or temporal), thalamus, hypothalamus, striatum, putamen, caudate nucleus, hippocampus, entorhinal cortex, basal ganglia, or deep cerebellar nuclei.

In some embodiments, the one or more modifications may increase distribution of an AAV particle to the CNS (e.g., the cortex) after intravenous administration. In some embodiments, the one or more modifications may increase distribution of an AAV particle to the CNS (e.g., the cortex) following focused ultrasound (FUS), e.g., coupled with the intravenous administration of microbubbles (FUS-MB), or MRI-guided FUS coupled with intravenous administration.

In some embodiments, the AAV capsid variant comprises (e.g., in loop IV) an amino acid sequence as set forth in Table 1. In some embodiments, the AAV capsid variant comprises (e.g., in loop TV) an amino acid sequence as set forth in Table 2A or Table 2B. In some embodiments, the AAV capsid variant comprises (e.g., in loop IV) an amino acid sequence set forth in Table 22. In some embodiments, the AAV capsid variant comprises (e.g., in loop IV) an amino acid sequence as set forth in Table 23.

TABLE 1
Exemplary Sequences

| Peptide Sequence | SEQ ID NO: |
|---|---|
| GSGSPHSKAQNQQT | 200 |
| GHDSPHKSGQNQQT | 201 |
| GSGSPHARMQNQQT | 202 |
| GSGSPHVKSQNQQT | 203 |
| GQDSPHKSGQNQQT | 204 |
| GSGSPHASRQNQQT | 205 |
| GSGSPHASRQNKQT | 206 |
| GSGSPHVKIQNQQT | 207 |
| GSGSPHSKAKNQQT | 208 |
| GSGSPHKKNQNQQT | 209 |
| GSGSPHVRMQNQQT | 210 |
| GSGSPHASRQKQQT | 211 |
| GHSSPHRSGQNQQT | 212 |
| GMRTYHLSGQNQQT | 213 |
| GSGSPHTRGQNQQT | 214 |
| GSGIIPVSSQNQQT | 215 |
| GSEYGHKSGQNQQT | 216 |
| GRGQNVSSVHRQQT | 217 |
| GSSHRFYGGQNQQT | 218 |
| GYFVAAWSGQNQQT | 219 |
| GSVLHSHAAQNQQT | 220 |
| GSGDLVVSTQNQQT | 221 |
| GSYGMAASGQNQQT | 222 |
| GLNHFGASGQNQQT | 223 |
| GSTGSHSAGQNQHT | 224 |
| GLAGHTVSGQNQQT | 225 |
| GIILGASSGQNQQT | 226 |
| GSGVSTYNIQNQQT | 227 |
| GSLVSVQTGQNQQT | 228 |
| GQSSPHRSGQNQQT | 229 |
| GREYGHKSGQNQQT | 230 |
| GHTLTLSSGQNQQT | 231 |
| GSITLIPSGQNQQT | 232 |
| GSNGFTALGQNQQT | 233 |
| GSGHSSHSVQNQQT | 234 |
| GSGIPQRSGKNQQT | 235 |
| GSGDTLHMLQNQQT | 236 |
| GERHTVLSGQNQQT | 237 |
| GSGMPQSHIQNQQT | 238 |
| GSGQLSGIGGNQQT | 239 |
| GSGQNRKPASFAQT | 240 |
| GSGSVSQLGQNQQT | 241 |
| GSDFLGTHGQNQQT | 242 |
| GQIVQNPSGQNQQT | 243 |
| GSGTQIPSQQNQQT | 244 |
| GSGQNQQSAREGLI | 245 |
| GSGLGMSTGQNQQT | 246 |
| GSGLPVLSGQNQQT | 247 |
| GSGHSIRTDQNQQT | 248 |
| GSGQSVQTVVNQQT | 249 |
| GSGQNRAQSRFQQT | 250 |
| GGGDLGRSSQNQQT | 251 |
| GGGTKMDSGQNQQT | 252 |
| GSGSPHPSRQNQQT | 253 |
| GSGQFTNAGMNQQT | 254 |
| GGRNGHTVGQNQQT | 255 |
| GSGFGPQTGQNQQT | 256 |
| GRIDSHTSGQNQQT | 257 |
| GYEVLGSSGQNQQT | 258 |
| GSVHLSVIGQNQQT | 259 |
| GFMSYKGSGQNQQT | 260 |
| GNIAGSVSGQNQQT | 261 |
| GSGSHRDVSQNQQT | 262 |
| GGLGSMSSGQNQQT | 263 |
| GSGHLPQSAQNQQT | 264 |
| GGVLVGGSGQNQQT | 265 |
| GTHPYTSSGQNQQT | 266 |
| GSGQNQQLKENRST | 267 |
| GSGQNQQTSPHNHT | 268 |
| GSGILYPQSQNQQT | 269 |
| GSGQNQQSNWITKT | 270 |
| GSGYTSLFLQNQQT | 271 |
| GSGVMTHVLQNQQT | 272 |
| GSVSDVRAGQNQQT | 273 |
| GSGQSHMATINQQT | 274 |
| GSGLSVHLAQNQQT | 275 |
| GSGLSHAIQQNQQT | 276 |
| GSGLSVQSGQNQQT | 277 |
| GSGHMTYREKNQQT | 278 |
| GSKGVPTPGQNQQT | 279 |
| GSGLLPLSSQNQQT | 280 |
| GNGLYAVSGQNQQT | 281 |
| GFNGSPSSGQNQQT | 282 |
| GSGQIRHSDQNQQT | 283 |
| GGQVAPSSGQNQQT | 284 |
| GSFSMHTHGQNQQT | 285 |
| GSGQNQQVIQGSNT | 286 |
| GRVLHSHAGQNQQT | 287 |
| GSGQNQQTSLQDQT | 288 |
| GSGLGRAPVQNQQT | 289 |
| GNGFSSASGQNQQT | 290 |
| GSGQMASRESNQQT | 291 |
| GPGLPNHSGQNQQT | 292 |
| GNIQWQGSGQNQQT | 293 |
| GMSAHMSSGQNQQT | 294 |
| GHSFVNRSGQNQQT | 295 |
| GRAVMDHSGQNQQT | 296 |
| GALTVMQSGQNQQT | 297 |
| GSGQRSPVLPNQQT | 298 |
| GSGQNGHLSLKQQT | 299 |
| GSLPRGTSDQNQQT | 300 |
| GVAGSLVSGQNQQT | 301 |
| GRGGIPQSGQNQQT | 302 |
| GSGQYASSIPNQQT | 303 |
| GIDFGRQSSQNQQT | 304 |
| GIFMQTPSGQNQQT | 305 |
| GSGQNQQIRLVDLI | 306 |
| GTREMPLSGQNQQT | 307 |
| GSRLVHVHGQNQQT | 308 |
| GSGRLVPNGPNQQT | 309 |
| GSGYLRESPQNQQT | 310 |
| GARIQNASGQKQQT | 311 |
| GLSNPMPSGQNQQT | 312 |
| GSTVQDTRGQNQQT | 313 |
| GPFGMPSSGQNQQT | 314 |
| GSGQNHGVLSNQQT | 315 |
| GSGYSMSQAQNQQT | 316 |
| GSGMLTHTLQNQQT | 317 |
| GRGSPHASRQNQQT | 318 |
| GLSWPSTSGQNQQT | 319 |
| GNSMERTSGQNQQT | 320 |
| GSGMSPSTLQNQQT | 321 |
| GSGHGQVLSQNQQT | 322 |
| GRGQIYSTGGNQQT | 323 |
| GVVAAHNSGQNQQT | 324 |
| GDSSLRHSGQNQQT | 325 |
| GSLVSQGAGQNQQT | 326 |
| GSLLQAHSGQNQQT | 327 |
| GSGHIYVGIQNQQT | 328 |
| GHHTTVQSGQNQQT | 329 |
| GSRQSKRNELNQQT | 330 |
| GSGQNQQHVSSPRT | 331 |
| GSSKELLWGQNQQT | 332 |
| GSLSTPSSGQNQQT | 333 |
| GSIGYAGQGQNQQT | 334 |
| GSGQNQRVSNSQQT | 335 |
| GSGYASHVQQNQQT | 336 |
| GSGEYSRSGQNQQT | 337 |
| GSVSTHSSGQNQQT | 338 |
| GSGQNQHSLGNYQT | 339 |
| GSGGLDTRGQNQQT | 340 |
| GNILHATSGQNQQT | 341 |
| GSGQSYTMTQNQQT | 342 |
| GSGQNQHSAPNSQT | 343 |
| GSGQNQQTMDHNRT | 344 |
| GSNGGVGIGQNQQT | 345 |
| GAGSIIPSGQNQQT | 346 |
| GSGQTHGGQHNQQT | 347 |
| GSNLSFQSGQNQQT | 348 |
| GATLQVHSGQNQQT | 349 |
| GSGFNQRSEQNQQT | 350 |
| GSGSLRDEDQNQQT | 351 |
| GSGDSITGKQNQQT | 352 |
| GSGQDRNIVQNQQT | 353 |
| GSGLSHSHQQNQQT | 354 |
| GSGQNQQTGMSSVK | 355 |
| GSVIHGISGQNQQT | 356 |
| GVVAHQPSGQNQQT | 357 |
| GSGPILGQLQNQQT | 358 |
| GSGHVPNSGLNQQT | 359 |
| GDAGVRSSGQNQQT | 360 |
| GSGSQLMSLQNQQT | 361 |
| GSGLDYSQRQNQQT | 362 |
| GSGQSSGRLINKQT | 363 |
| GSSVSPSSGQNQQT | 364 |
| GSGQVVGLSGNQQT | 365 |
| GSNMGVPLGQNQQT | 366 |
| GSFYPSSTGQNQQT | 367 |
| GSGQNQQTRLTDLT | 368 |
| GPTNGRSSGQNQQT | 369 |
| GSGLLHGKLQNQQT | 370 |
| GANMGHVSGQNQQT | 371 |
| GSGQNQQSGRGDLT | 372 |
| GSHGHYASGQKQQT | 373 |
| GSGDLRISPQNQQT | 374 |
| GSGMPVILGQNQQT | 375 |
| GRGVITSSGQNHQT | 376 |
| GSGHSVSGPQNQQT | 377 |
| GSRNGHIVGRNQQT | 378 |
| GAGVHMVSGQNQQT | 379 |
| GSGQNHRPSVLQQT | 380 |
| GSGSPRDSIQNQQT | 381 |
| GSGQGIHSSVNQQT | 382 |
| GSGQQLSITPNQQT | 383 |
| GGYHSQTSGQNQQT | 384 |
| GSLHHDNHGQNQQT | 385 |
| GIMARDSSGQNQQT | 386 |
| GVVHITNSGQNQQT | 387 |
| GSGQNQHSAPFNQT | 388 |
| GSGQTSGLKQNQQT | 389 |
| GSGQNQQTSLSNTA | 390 |
| GSGQNQAVHNKSQT | 391 |
| GVHTHLPSGQNQQT | 392 |
| GHLTMHNSGQNHQT | 393 |
| GSGSSSRPYQNQQT | 394 |
| GILLATPSGQNQQT | 395 |
| GSGQNAGSFPNQQT | 396 |
| GSRDGHTVGQNQQT | 397 |
| GSLLISTSGQNQQT | 398 |
| GSGAMPSHGQNQQT | 399 |
| GALVSPISGQNQQT | 400 |
| GSLSSHGVGQNQQT | 401 |
| GSGQNQQASLAMRT | 402 |
| GPGLGSHSGQNQQT | 403 |
| GHDSQHKSGQNQQT | 404 |
| GSGLILSATQNQQT | 405 |
| GSGQVVAHVGNQQT | 406 |
| GSGLRTMTTQNQQT | 407 |
| GSGQVGRLLQNQQT | 408 |
| GSGQLSHQSVNQQT | 409 |
| GSGDRYQTLQNQQT | 410 |
| GSGQNQQLKSSAQT | 411 |
| GSGQNQYSIPVAQT | 412 |
| GSGERLHLIQNQQT | 413 |
| GSGHNQQVRTAPNT | 414 |
| GGLSHVMSGQNQQT | 415 |
| GSGQSHRDVLNQQT | 416 |
| GSGQNLAGRMDQQT | 417 |
| GSGQNQQTNRGNPM | 418 |
| GSGQSYQRDHNQQT | 419 |
| GSLLSAGMGQNQHT | 420 |
| GSGQNQQTAIYRNI | 421 |
| GSGQNQQTSGITNC | 422 |
| GMTSHSVSGQNQQT | 423 |
| GSSQSTGYQPNQQT | 424 |
| GSLKPTTLGQNQQT | 425 |
| GRMESLGSGQNQQT | 426 |
| GSGQNQQTALGVKC | 427 |
| GAMVSHSSGQNQQT | 428 |
| GSGQNQQRNSDSVT | 429 |
| GSGQSMILHLNQQT | 430 |
| GSGQVHQAEVNQQT | 431 |
| GSGQNQSQNHLQQT | 432 |
| GSLLTTASGQNQQT | 433 |
| GSGLIRTAAQNQQT | 434 |
| GSGQNQQTVSRQST | 435 |
| GSGQYANHGINQQT | 436 |
| GSRSTGPSGQNQQT | 437 |
| GRGVQQKLQQNQQT | 438 |
| GSGQNQQVHLSTGT | 439 |
| GSGQNQQLSAKSST | 440 |
| GSGYKAARPQNQQT | 441 |
| GSAGISPSGQNQQT | 442 |
| GSGQNRAHAFLQQT | 443 |
| GSGLSGITMQNQQT | 444 |
| GPGSAHSSGQNQQT | 445 |
| GSSHIQALGQNQQT | 446 |
| GSGVHGVSSQNQQT | 447 |
| GSSGRDMGGQNQQT | 448 |
| GERAFPTSGQNQQT | 449 |
| GGRIVSLSGQNQQT | 450 |
| GSGQNSYSHISQQT | 451 |
| GLGYPGSSGQNQQT | 452 |
| GSGPQSHTGQNQQT | 453 |
| GSGQNQQLSRDAST | 454 |
| GSGQILHSVPNQQT | 455 |
| GSGFHTDSRQNQQT | 456 |
| GSGQSHSLATNQQT | 457 |
| GSGQNQQTLSKPWT | 458 |
| GSGHAAISQQNQQT | 459 |
| GSGQNQQQIGGNST | 460 |
| GGGPMAGSGQNQQT | 461 |
| GMRMEYQSGQNQQT | 462 |
| GSGQNQQGTLLHQT | 463 |
| GSGQNQRSSGGVQT | 464 |
| GSGQNQRGALATQT | 465 |
| GSGTVHAATQNQQT | 466 |
| GSRMIQQFGQNQQT | 467 |
| GSSSPGASGQNQQT | 468 |
| GHPSPHVSGQNQQT | 469 |
| GSGSHHASRQNQQT | 470 |
| GAVGHSYSGQNQQT | 471 |
| GSRSQYDIGQNQQT | 472 |
| GSGQGPQERGNQQT | 473 |
| GSIAHVGTGQNQQT | 474 |
| GSGQNQQKQNHGNT | 475 |
| GSGQNQQALGSQRT | 476 |
| GSGAITHMPQNQQT | 477 |
| GSGQRNPLLLNQQT | 478 |
| GSSGIPVSHQNQQT | 479 |
| GVHSLIPSGQNQQT | 480 |
| GVIVLHGSGQNQQT | 481 |
| GGTRVVDSGQNQQT | 482 |
| GSGGVTYQSQNQQT | 483 |
| GSGQNQAGHGPGQT | 484 |
| GSGQLVTSGPNQQT | 485 |
| GSGIAAQRTQNQQT | 486 |
| GSTPAGVGGQNQQT | 487 |
| GSGQNQQTSTGVHS | 488 |
| GSGQIRQLVDNQQT | 489 |
| GSLIGMQSGQNQQT | 490 |
| GSGQIKGKMDNQQT | 491 |
| GSGSDMSSWQNQQT | 492 |
| GRGQNQQHTGLATT | 493 |
| GSGQNQQTLYSSNT | 494 |
| GSGQTQVLKSNQQT | 495 |
| GSRTLSNVGQNQQT | 496 |
| GSGVQHSLPQNQQT | 497 |
| GNYLHQASGQNQQT | 498 |
| GSGGTSVHQQNQQT | 499 |
| GMDHSRPSGQNQQT | 500 |
| GSGQNQQSMGTFTT | 501 |
| GSGQNQQTPLRPPT | 502 |
| GSGQNQHHSVSQQT | 503 |
| GSGQLRSLSINQQT | 504 |
| GSGSPRQLSQNQQT | 505 |
| GSGQNQQTTASSHT | 506 |
| GRGQVVSTHQNQQT | 507 |
| GSAQVSMVGQNQQT | 508 |
| GSSILVTIGKNQQT | 509 |
| GFAHQASSGQNQQT | 510 |
| GSGQPVLSISNQQT | 511 |
| GSGQSHRSELNQQT | 512 |
| GSSVGSPIGQNQQT | 513 |
| GSGMPIRNVQNQQT | 514 |
| GSSTRVDSGQNQQT | 515 |
| GSGQNQQTAMRSTT | 516 |
| GSGQNQQHSSSHLT | 517 |
| GSRNGHAVGQNQQT | 518 |
| GLGAYQSSGQNQQT | 519 |
| GPGLSGHSGQNQQT | 520 |
| GSTGIVSSGQNQQT | 521 |
| GSRTTQVIGQNQQT | 522 |
| GSGLLHRAQQNQQT | 523 |
| GSGQNAQQAAAQQT | 524 |
| GSGQNQQSALRTQT | 525 |
| GSGELSDIRQNQQT | 526 |
| GSGLLYHDQQNQQT | 527 |
| GSGQNQHYSLHKQT | 528 |
| GSGHSPLPQQNQQT | 529 |
| GNGHSMRPNQNQQT | 530 |
| GSGLKWSTLQNQQT | 531 |
| GSGQMGRQAVNQQT | 532 |
| GSGQNQQTSGVLIL | 533 |
| GSGQNQQALHNPHT | 534 |
| GSGQNQQVIPNSKT | 535 |
| GSPLQDRVGQNQQI | 536 |
| GSGQNQYSSINPQT | 537 |
| GAMTVIISGQNQQT | 538 |
| GSGQNQQLQTLIRT | 539 |
| GSGLRQTSQQNQQT | 540 |
| GSGQNQQTGLRQQT | 541 |
| GSGQTRQMKDNQQT | 542 |
| GSGQNHGLQSGQQT | 543 |
| GSGQSHRQPENQQT | 544 |
| GSGQDRHIVQNQQT | 545 |
| GSGQNQQLPHSNLT | 546 |
| GSGQLSVPYDNQQT | 547 |
| GSGRNPQTQPLQQT | 548 |
| GSGQPYSTGLNQQT | 549 |
| GSGQNQQTHGGLRD | 550 |
| GAYGMVSSGQNQQT | 551 |
| GSGIQSSYSQNQQT | 552 |
| GPRLSDQSGQNQQT | 553 |
| GSGQNQQTHPSPCT | 554 |
| GSGQSFQMHTNQQT | 555 |
| GSGQNQQTGNPKHT | 556 |
| GFSSAVHSGQNQQT | 557 |
| GSGQNQQTSMSNAT | 558 |
| GSGQDMKQHHNQQT | 559 |
| GLRLSTPSGQNQQT | 560 |
| GSGQNQQTSVYMNT | 561 |
| GSGQNQYSQSSMQT | 562 |
| GSGQNQQSMADHTT | 563 |
| GWERSFVSGQNQQT | 564 |
| GLLAGKSSGQNQQT | 565 |
| GKSFVPQSGQNQQT | 566 |
| GSGQMQSAGSNQQT | 567 |
| GSDQNQRLTSSMQT | 568 |
| GESRAVLSGQNQQT | 569 |
| GSVFGVPSGQNQQT | 570 |
| GSGLPDRNLQNQQT | 571 |
| GSGTHNSAIQNQQT | 572 |
| GSGMIIASMQNQQT | 573 |
| GGITWTDSGQNQQT | 574 |
| GSGQNQQASGRQQT | 575 |
| GSGQNQQPHLKSLT | 576 |
| GPPQHMTSGQNQQT | 577 |
| GSGQNQQASLPSRT | 578 |
| GSGQIVSTQTNQQT | 579 |
| GSGKGHSAGQNQQT | 580 |
| GSGQNTRLQLGQQT | 581 |
| GSVGSRPVGQNQQT | 582 |
| GSSHTLALGQNQQT | 583 |
| GMYEYSQSGQNQQT | 584 |
| GNGQNQQHSILHGT | 585 |
| GSGYNQPHLQNQQT | 586 |
| GPLVNASSGQNQQT | 587 |
| GSGQNQQVLTTART | 588 |
| GSGQNQHSVHNDQT | 589 |
| GAGLIMHSGQNQQT | $90 |
| GMGRHSASGQNQQT | 591 |
| GSHSQSGHGQNQQT | 592 |
| GSSTTIVSGQNHQT | 593 |
| GRHLVTASGQNQQT | 594 |
| GSGQNQQHANLNQT | 595 |
| GSGSTRSKAQNQQT | 596 |
| GSGQNKQMLSGNTT | 597 |
| GSGQVHNPTQNQQT | 598 |
| GSGQNQQIPHVHQT | 599 |
| GSLHAGLSGQNQQT | 600 |
| GPAQHGTSGQNQQT | 601 |
| GEKAVTSSGQNQQT | 602 |
| GSGQNQQTMANGQR | 603 |
| GSGSPHSKDQNQQT | 604 |
| GSFSMGYGGQNQQT | 605 |
| GSGTHLVSLQNQQT | 606 |
| GSGQMQPHVQNQQT | 607 |
| GSGQNQQVAGLNNT | 608 |
| GSSQNQQHDMRLRT | 609 |
| GPASLPISGQNQQT | 610 |
| GSGQNQQPPLATRT | 611 |
| GSSRVPVSGQNQQT | 612 |
| GSGQNQQTNLGHTT | 613 |
| GSGQNQQLVSRVQT | 614 |
| GPNSYPVSGQKQQT | 615 |
| GHAHYQASGQNQQT | 616 |
| GSGQALLSTGNQQT | 617 |
| GSGQLPRQMTNQQT | 618 |
| GSGFPKSTEQNQQT | 619 |
| GSRETSLSGQNQQT | 620 |
| GSGQNQQGTGVSHT | 621 |
| GSRTVPVYGQNQQT | 622 |
| GSNAQSAHGQNCQT | 623 |
| GAFHLAASGQNQQT | 624 |
| GSGQYRSSSDNQQT | 625 |
| GSGQVYISTPNQQT | 626 |
| GSGVSTQLLQNQQT | 627 |
| GSGQLGLSVINQQT | 628 |
| GSGSNMRLSQNQQT | 629 |
| GSGQNLHSGLPQQT | 630 |
| GSSHTLALGQNKQT | 631 |
| GSGQNQHSLPAHRT | 632 |
| GSGQNQGTVYPNQT | 633 |
| GSGQNQQPSLRQST | 634 |
| GSGQNARLKDNQQT | 635 |
| GHAGSTGSGQNQQT | 636 |
| GSGQALSSSGNQQT | 637 |
| GSGASESHRQNQQT | 638 |
| GVGVITSSGQNQQT | 639 |
| GSLYGQSLGQNQQT | 640 |
| GSGQMSDVHGNQQT | 641 |
| GSGQNQQHSSKATT | 642 |
| GSGQNQQTSVSQQT | 643 |
| GSGQKMWKLDNQQT | 644 |
| GSGQNVSMQVNQQT | 645 |
| GSGQNQRATLSNQT | 646 |
| GSGQASSKSANQQT | 647 |
| GSGKNQTPIPKGQT | 648 |
| GSGQNQQTRQEGST | 649 |
| GASSLATSGQNQQT | 650 |
| GSGQRGSLTENQQT | 651 |
| GSEQTRQRGQNQQT | 652 |
| GSGQNQQTLTASKE | 653 |
| GSGTSGKTGKNQQT | 654 |
| GQLVTFTSGQNQQT | 655 |
| GSGQNQQSANKILT | 656 |
| GSGQNQQHHSSHTT | 657 |
| GSGQNQKGMQPNQT | 658 |
| GSGQLVSGLYNQQT | 659 |
| GSSVGVPSGQNQQT | 660 |
| GSGQNQQWDSRRQT | 661 |
| GSEQTRQSGQNQQT | 662 |
| GSGIGSHIPQNQQT | 663 |
| GSGQNQRLHGVDQT | 664 |
| GEVSRVLSGQNQQT | 665 |
| GSGQNQQKVSPLLT | 666 |
| GSGLALERSQNQQT | 667 |
| GPDRIGSSGQNQQT | 668 |
| GSGQNQDHQNKQQT | 669 |
| GSGQNQQTALYNNT | 670 |
| GSGAVHLTAQNQQT | 671 |
| GSLVSTQSGQNQQT | 672 |
| GSGVSARMVQNQQT | 673 |
| GSGQTRMPLANQQT | 674 |
| GSGISSRNMQNQQT | 675 |
| GSGEKVHSGQNQQT | 676 |
| GSGQNQQKLSSMST | 677 |
| GSGQNQQTGQHMRV | 678 |
| GSGMIHTTAQNQQT | 679 |
| GSGQNWPALKGQQT | 680 |
| GASHMSISGQNQQT | 681 |
| GSDQNQQLGYSKQT | 682 |
| GIPSIRESGQNQQT | 683 |
| GSGIPSVKFQNQQT | 684 |
| GSGQNQQTSVSQNV | 685 |
| GSGQNQQIGESRMT | 686 |
| GSGSSSMSFQNQQT | 687 |
| GSGQKQERAVSKQT | 688 |
| GCTTRLNSGQNQQT | 689 |
| GSGQNQQIISTKIT | 690 |
| GSGQNQQKSLNGNT | 691 |
| GSGIPAPRLQNQQT | 692 |
| GSGQIRESMGNQQT | 693 |
| GSGQNSGVHENQQT | 694 |
| GSGQNIGHSLPQQT | 695 |
| GSGERSISVQNQQT | 696 |
| GSGLKPNVLQNQQT | 697 |
| GSGQVAYAQGNQQT | 698 |
| GSGQSSYGSGNQQT | 699 |
| GSGQNQAMTHGDQT | 700 |
| GSGQNQALVSMGQT | 701 |
| GSGQNPSFMRGQQT | 702 |
| GSGQNQQSHLRTNT | 703 |
| GYTRLETSGQNQQT | 704 |
| GSGQSYDMRGNQQT | 705 |
| GSRTTQDIGQNQQT | 706 |
| GSGHPYKAAQNQQT | 707 |
| GRLSNAHGGQNQQT | 708 |
| GSGQNQRAVINDQT | 709 |
| GGSHTYGGGQNQQT | 710 |
| GSSVNSMIGQNQQT | 711 |
| GNSSMMGSGQNQQT | 712 |
| GNRDRPSSGQNQQT | 713 |
| GSGNMHASRQNQQT | 714 |
| GFIFPKVSGQNQQT | 715 |
| GSGQNQQLKNSTST | 716 |
| GSGQNQQSQYMPRT | 717 |
| GSGQRMADIGNQQT | 718 |
| GSGQNQSHYPSQQT | 719 |
| GSDGKMHRGQNQQT | 720 |
| GSGSVGFIGQNQQT | 721 |
| GLHGMTLSGQNQQT | 722 |
| GSDQSKRGDSNQQT | 723 |
| GSLFLATGGQNQQT | 724 |
| GSGQNQQPSAFSKT | 725 |
| GSGQLPQSGLNQQT | 726 |
| GSGSKQNALQNQQT | 727 |
| GSGQRRELSQNQQT | 728 |
| GSGQREPKASNQQT | 729 |
| GSGQNQQHPSTQQT | 730 |
| GSQSTLGLGQNQQT | 731 |
| GSGQNQQMPGLSST | 732 |
| GSGQNQQTVGGKNL | 733 |
| GSSREFHSGQNQQT | 734 |
| GSGQNQQTVPSNLV | 735 |
| GSGQNAYSSQAQQT | 736 |
| GSGQNKDHSTRRQT | 737 |
| GQLGSVGSGQDQQT | 738 |
| GSGQHAAPGHNQQT | 739 |
| GSGQNQQTSQSPPT | 740 |
| GSGNYRDHEQNQQT | 741 |
| GSGQHSNQHVNQQT | 742 |
| GSGQTARNGINQQT | 743 |
| GSGQNQQHYGSQGT | 744 |
| GSGSPQASRQNQQT | 745 |
| GSGFSHSMGKNQQT | 746 |
| GSGQSHSLETNQQT | 747 |
| GTEQTRQSGQNQQT | 748 |
| GSGRHLASVQNQQT | 749 |
| GLGSKNHSGQNQQT | 750 |
| GSGQNQQTSHFPSA | 751 |
| GSGQLSGTPQNQQT | 752 |
| GSGQNQQAPHKKET | 753 |
| GSGQNQQTLRGSLE | 754 |
| GSIAMTSHGQNQQT | 755 |
| GSPGVSPSGQNQQT | 756 |
| GSGQNQQTGSSSRV | 757 |
| GSGQHLPLLGNQQT | 758 |
| GSDHSHRGGQNQQT | 759 |
| GSGIVTKLGQNQQT | 760 |
| GSGQDVTKTGNQQT | 761 |
| GSGQNQQSHGRIGT | 762 |
| GSGQNQQINHRSPT | 763 |
| GSGDDSRVGQNQQT | 764 |
| GSGQSTLKRINQQT | 765 |
| GSGSQHSKAQNQQT | 766 |
| GSGQNQQHASSNNT | 767 |
| GSRTYQVSGQNQQT | 768 |
| GSGQNQGLLSSPQT | 769 |
| GSGGGLQHNQNQQT | 770 |
| GSGQNQQTTAATRM | 771 |
| GSGQNQRASILVQT | 772 |
| GSGQNLGLLGAQQT | 773 |
| GSLDLGRSGQNQQT | 774 |
| GNSQVKVSGQNQQT | 775 |
| GSSGSHQYGQNQQT | 776 |
| GSGQNQQQRDGTLT | 777 |
| GRGQHVSVANNQQT | 778 |
| GDSSSRISGQNQQT | 779 |
| GSGQNQQHSLSSQT | 780 |
| GSLMDVERGQNQQT | 781 |
| GSIQYQSSGQNQQT | 782 |
| GLGSKNPSGQNQQT | 783 |
| GSGQLVLTLQNQQT | 784 |
| GSGQNQQTSQPLPG | 785 |
| GSGQNQQNLGKLNT | 786 |
| GTTAHQPSGQNQQT | 787 |
| GSGQNRAQIGTQQT | 788 |
| GSGQYVHVSSNQQT | 789 |
| GSGQNQQTAHAFNI | 790 |
| GSGQNQRTMVATQT | 791 |
| GSGQNPIRGAMQQT | 792 |
| GSGYVITGSQNQQT | 793 |
| GRGPKQSNIQNQQT | 794 |
| GSGQNQQTMLGKPC | 795 |
| GSGQNQQVGSTVRT | 796 |
| GNVTTQKSGQNQQT | 797 |
| GSGNPVSHLQNQQT | 798 |
| GSLSHMESGQNQQT | 799 |
| GRAPTNLSGQNQQT | 800 |
| GSGQNQQTVMTARA | 801 |
| GSGMPASRLQNQQT | 802 |
| GVVRNHQSGQNQQT | 803 |
| GSGQNQHSVQVRQT | 804 |
| GSGQNTGHLTMQQT | 805 |
| GSGQNQQYAGKILT | 806 |
| GSGNPHVRNQNQQT | 807 |
| GSGQNGGSSNRQQT | 808 |
| GSGQRLSQGVNHQT | 809 |
| GSGQNAHAKEGQQT | 810 |
| GSSPAPNSGQNQQT | 811 |
| GLAHKTSSGQNQQT | 812 |
| GSGQNQQTPGAHKT | 813 |
| GSGQNQQSLSGSFT | 814 |
| GSGQNQQSTGTSRT | 815 |
| GSGQNQQTVQSNLV | 816 |
| GSGQNQQLGSRQCT | 817 |
| GSGQNQYLRLELQT | 818 |
| GSGQNQQTSPRLQT | 819 |
| GSGQNQQTTSSNMT | 820 |
| GTASTYNSGQNQQT | 821 |
| GSGQNQQTMPQHKI | 822 |
| GSGQSHLHTGNQQT | 823 |
| GVKGVGHSGQNQQT | 824 |
| GSGKVIKQSQNQQT | 825 |
| GSGQNQQTALEKSL | 826 |
| GSGYKDTYGQNQQT | 827 |
| GSGQNQQSGTFLST | 828 |
| GSGQNTGQHMMQQT | 829 |
| GSGKNQQRPGLDQT | 830 |
| GSGQSREISLNQQT | 831 |
| GTPTSPSSGQNQQT | 832 |
| GKPAGGLSGQNQQT | 833 |
| GSGQNHRSADMQQT | 834 |
| GSGQNQQTLPSLSL | 835 |
| GSPYMGATGQNQQT | 836 |
| GSGHAKAVGQNQQT | 837 |
| GHMKGVTSGQNQQT | 838 |
| GSGQNQKILTLDQT | 839 |
| GSGQNQQTKVGHSA | 840 |
| GIARTTISGQNQQT | 841 |
| GSGQNQQTSVGFRT | 842 |
| GSGQNQQTMIANIR | 843 |
| GDMTRSSSGQNQQT | 844 |
| GSGHMSDLRQNQQT | 845 |
| GRGAVMASGQNQQT | 846 |
| GSGQNQQLSGKSVT | 847 |
| GSHTLVVSGQNQQT | 848 |
| GSGPWSAGLQNQQT | 849 |
| GSGQHSPRALNQQT | 850 |
| GSGQNQQPNSGSMT | 851 |
| GSGLAHLGGQNQQT | 852 |
| GSSVRYEPKQNQQT | 853 |
| GSGQNQQARPLELT | 854 |
| GSGQPRSTGINQQT | 855 |
| GSGQNQANWVKVQT | 856 |
| GSGHLFQSGQNQQT | 857 |
| GSGQNRGISISQQT | 858 |
| GSGTHYDNRQNQQT | 859 |
| GSGQNQQTSTTPLP | 860 |
| GSGQVHASQVNQKT | 861 |
| GSSGHRESGQNQQT | 862 |
| GLSAEKSSGQNQQT | 863 |
| GSGQEHRSLANQQT | 864 |
| GSGQTVVRIANQQT | 865 |
| GSGQNVSSVHRQQT | 866 |
| GSGASRMSIQNQQT | 867 |
| GVAFIGSSGQNQQT | 868 |
| GSGQNQQTVPTRQT | 869 |
| GSGQAAKSSQNQQT | 870 |
| GSGQNQQVAIRTST | 871 |
| GSVHMQNAGQNQQT | 872 |
| GSGMRQAGVQNQQT | 873 |
| GSGQNQQVGGKTVT | 874 |
| GVHDMRVSGQNQQT | 875 |
| GSGQHVSVANNQQT | 876 |
| GSAAMSVRGQNQQT | 877 |
| GVSRGGPSGQNQQT | 878 |
| GSGQMVHTIGNQQT | 879 |
| GRGGSMAETQNQQT | 880 |
| GSGHTNPTRQNQQT | 881 |
| GSGEAARYEQNQQT | 882 |
| GSGQNERHLVLQQT | 883 |
| GSGQNQQSKQQVLT | 884 |
| GSGQARAHRGNQQT | 885 |
| GSGQNQQPLDTSRT | 886 |
| GSGQNQQLANMVTT | 887 |
| GSGQMKDLHRNQQT | 888 |
| GSGQNQHLSSFVQT | 889 |
| GSGQNQQPSSRVTT | 890 |
| GSGQNQQLAITLGT | 891 |
| GSGQNQQTVGNPAT | 892 |
| GSGQNQGRAHMQTP | 893 |
| GSGQLIASVVNQQT | 894 |
| GSSVRSLVGQNQQT | 895 |
| GGAGSAHSGQNQQT | 896 |
| GSDQNQQTMSSTRT | 897 |
| GSGQNQQMAGAFRT | 898 |
| GSLGNLQRGQNQQT | 899 |
| GSGPSISHGQNQQT | 900 |
| GSGQNQQSSENVQT | 901 |
| GSGQNQQTGQATHN | 902 |
| VSGSPHSKAQNQQT | 903 |
| CSGSPHSKAQNQQT | 904 |
| GSGSPHRKAQNQQT | 905 |
| GRGSPHSKAQNQQT | 906 |
| GSGSPHSKAQNKQT | 907 |
| GSGSPHSKAQTQQT | 908 |
| GSGSTHASRQNQQT | 909 |
| GSGSPHKYGQNQQT | 910 |
| GSGSPHKEGQNQQT | 911 |
| VSGSPHKFGQNQQT | 912 |
| GSGSPHSKAQNHQT | 913 |
| GSGSPHSKAQHQQT | 914 |
| GSGSPHKTYQNQQT | 915 |
| VSGSPHASRQNQQT | 916 |
| GSGSPHKFGKNQQT | 917 |
| GSGSPHASRQNQHT | 918 |
| GSHSPHKSGQNQQT | 919 |
| GSGQNQQRRMSPST | 920 |
| GSGSPHSKPQNQQT | 921 |
| GSGSPHKFGQKQQT | 922 |
| VSGSPHGARQNQQT | 923 |
| GSGSPHSKAQKQQT | 924 |
| GSHSPHKRGQNQQT | 925 |
| GSGQNRQRLKGLET | 926 |
| GSGSPHKLGQNQQT | 927 |
| GSGSPHKTSKNQQT | 928 |
| GSGSPHKIGQNQQT | 929 |
| GSGQDSPHVRNQQT | 930 |
| GSGSPHKTSQNQQT | 931 |
| GSGSPHASRKNQQT | 932 |
| GSHSPHKSGQKQQT | 933 |
| GSGSPHKTSQKQQT | 934 |
| GSGSPHVRGQNKQT | 935 |
| GSGSPHKTTQNQQT | 936 |
| GSGPVRALRQNQQT | 937 |
| GSGSPHVRGQKQQT | 938 |
| CSGSPHKTSQNQQT | 939 |
| CSHSPHKSGQNQQT | 940 |
| GHDSPHKSGQNQQ | 1800 |
| GSGSPHSKAQNQQ | 1801 |
| GSGSPHSKAQNRH | 1802 |
| GSGSPHSKVQNQQ | 1803 |
| MSGSPHSKAQNQQ | 1804 |
| GRGSPHSKAQNQQ | 1805 |
| RNGSPHSKAQNQQ | 1806 |
| GSGSPHSKARDQQ | 1807 |
| GSGSPHSKAPNLQ | 1808 |
| TSGSPHSKAQNQQ | 1809 |
| GSGSPHSKAHVRQ | 1810 |
| GSGSPHSKAPNQH | 1811 |
| ISGSPHSKAQNQQ | 1812 |
| GPGSPHSKAHNQQ | 1813 |
| GSGSPHSKTQSQQ | 1814 |
| ESGSPHSKAQNQQ | 1815 |
| GSGSPHSKAQPAK | 1816 |
| SSGSPHSKAQNQQ | 1817 |
| GNGSPHSKAQNQQ | 1818 |
| GSGSPHSKSQTQQ | 1819 |
| ASGSPHSKAQNQQ | 1820 |
| GSGSPHSKAQNLA | 1821 |
| GSGSPHSKSQNQL | 1822 |
| NSGSPHSKAQNQQ | 1823 |
| GSGSPHSKAQGQQ | 1824 |
| VSGSPHSKAQNQQ | 1825 |
| GSGSPHSKALNRQ | 1826 |
| LSGSPHSKAQNQQ | 1827 |
| GSGSPHSKAHNQQ | 1828 |
| GSGSPHSKTQNQQ | 1829 |
| GGGSPHSKAQTQQ | 1830 |
| GSGSPHSKAQNPP | 1831 |
| GSGSPHSKAQNLQ | 1832 |
| GGGSPHSKAQNQQ | 1833 |
| GSGSPHSKAQYQQ | 1834 |
| GGGSPHSKAQNKQ | 1835 |
| GSGSPHSKAQDQE | 1836 |
| KSGSPHSKAQNQQ | 1837 |
| GGGSPHSKAQNQL | 1838 |
| GSGSPHSKAQNHQ | 1839 |
| GSGSPHSKAQDQQ | 1840 |
| GGGSPHSKSQNQL | 1841 |
| GNGSPHSKAQNKQ | 1842 |
| GSGSPHSKGHWQQ | 1843 |
| GSGSPHSKAPNQQ | 1844 |
| GSGSPHSKAQNQL | 1845 |
| GSGSPHSKRPEQQ | 1846 |
| GSGSPHSKAQRTM | 1847 |
| GNGSPHSKAQNQH | 1848 |
| HSGSPHSKAQNQQ | 1849 |
| GGGSPHSKALNQQ | 1850 |
| GSGSPHSKALHQH | 1851 |
| GTGSPHSKAQNHQ | 1852 |
| GSGSPHSKAQRRI | 1853 |
| GSGSPHSKAQYIH | 1854 |
| GGGSPHSKAHNQQ | 1855 |
| GSGSPHSKAQKFE | 1856 |
| ESGSPHSKAQNHQ | 1857 |
| GSGSPHSKAQFPS | 1858 |
| PSGSPHSKAQNQQ | 1859 |
| GNGSPHSKAQNPL | 1860 |
| GGGSPHSKAQSQQ | 1861 |
| GSGSPHSKAQAIK | 1862 |
| GSGSPHSKGQNRQ | 1863 |
| GSGSPHSKAQSQQ | 1864 |
| GSVSPHGKAQNQL | 1865 |
| ASGSPHSKAQNQL | 1866 |
| RSGSPHSKAQNQQ | 1867 |
| GSGSPHSKAQYQH | 1868 |
| GSGSPHTKAQNPQ | 1869 |
| GSGSPHSKGQNPP | 1870 |
| GSGSPHSKAQHQL | 1871 |
| GSGSPHSKAQSPP | 1872 |
| GSGSPHSKAQAKL | 1873 |
| GSGSPHSKTKSQQ | 1874 |
| GSGSPHSKAQDRP | 1875 |
| GIGSPHSKAQNLG | 1876 |
| GSGSPHSKAQAFH | 1877 |
| GSGSPHSKAQKQQ | 1878 |
| GSGSPHSKAQNAQ | 1879 |
| WSGSPHSKAQNQQ | 1880 |
| GSGSPHSKAHNQL | 1881 |
| GNGSPHSKAQNHQ | 1882 |
| GGGSPHSKAQNLQ | 1883 |
| GSGSPHSKAQKLN | 1884 |
| GGGSPHSKSQNQH | 1885 |
| GSGSPHSKSQNVQ | 1886 |
| GSGSPHSKAQAQQ | 1887 |
| DSGSPHSKAQNQQ | 1888 |
| ASGSPHSKAPNQQ | 1889 |
| GSGSPHSKAQTPP | 1890 |
| IDGSPHSKAQNQQ | 1891 |
| GSGSPHNKAQNHQ | 1892 |
| GSGSPHSKAQPPA | 1893 |
| GSGSPHSKAQERP | 1894 |
| GSGSPHSKAQDLQ | 1895 |
| GGGSPHSKAQNPP | 1896 |
| GSGSPHSKAQAMH | 1897 |
| GSGSPHSKALNQQ | 1898 |
| GSGSPHSKAQHPS | 1899 |
| GLGSPHSKSQNQQ | 1900 |
| GTGSPHSKAQNQQ | 1901 |
| GSGSPHSKAPGLQ | 1902 |
| GSGSPHSKAQGIR | 1903 |
| GSGSPHSKAQAPA | 1904 |
| GSGSPHSKSQSQQ | 1905 |
| GSGSPHSKAQIPP | 1906 |
| GSGSPHSKAQTQL | 1907 |
| GSGSPHSKAQAPS | 1908 |
| DAGSPHSKAQNQQ | 1909 |
| GSGSPHSKGQNQQ | 1910 |
| DGGSPHSKAQNQQ | 1911 |
| ASGSPHSKAHNQQ | 1912 |
| GSGSPHSKAQNTY | 1913 |
| GSGSPHSKSQNQH | 1914 |
| GGGSPHSKAQDKQ | 1915 |
| GSGSPHSKAQNHL | 1916 |
| GSGSPHSKAQIGM | 1917 |
| GSGSPHSKALNKQ | 1918 |
| GGGSPHSKAQNPQ | 1919 |
| GTGSPHSKAPNQL | 1920 |
| GSGSPHSKAQLQQ | 1921 |
| GGGSPHSKAQYQQ | 1922 |
| GGGSPHSKAQHQQ | 1923 |
| GSGSPHSKAQRMS | 1924 |
| GSGSPHSKAQGIL | 1925 |
| GSGSPHSKAQDRQ | 1926 |
| GSGSPHSKARDWQ | 1927 |
| GSGSPHSKAQNTH | 1928 |
| GSGSPHSKAQERS | 1929 |
| GSGSPHSKAQNYQ | 1930 |
| GSGSPHSKAQRTC | 1931 |
| GSGSPHSKAQIGH | 1932 |
| GSGSPHSKAQGAI | 1933 |
| GSGSPHSKAQVPP | 1934 |
| GSGSPHSKAQVQQ | 1935 |
| GSGSPHSKALMRQ | 1936 |
| GSGSPHSKAQYSV | 1937 |
| GSGSPHSKVPNLQ | 1938 |
| GSGSPHSKAQAIT | 1939 |
| GSGSPHSKAQKTL | 1940 |
| GSGSPHSKAQNQW | 1941 |
| GSGSPHSKAQLHH | 1942 |
| GSGSPHSKAQNII | 1943 |
| MEGSPHSKAQNQQ | 1944 |
| GSGSPHSKAQGHH | 1945 |
| GSGSPHSKAQSKV | 1946 |
| GSGSPHSKAQLPS | 1947 |
| GSGSPHSKAIGKQ | 1948 |
| GGGSPHSKSQNQQ | 1949 |
| GSGSPHSKAQAIH | 1950 |
| GSGSPHSKAQHGL | 1951 |
| GSGSPHSKAQFMC | 1952 |
| VSGSPHSKAQGQQ | 1953 |
| GGGSPHSKAQNQM | 1954 |
| GSGSPHSKAQHLQ | 1955 |
| ENGSPHSKAQNQQ | 1956 |
| GSGSPHSKTQNHQ | 1957 |
| GSGSPHSKAQPAR | 1958 |
| GSGSPHSKAQSLQ | 1959 |
| GSGSPHSKSQSQL | 1960 |
| GSASPHSKAHSQQ | 1961 |
| GSGSPHSKAQMPS | 1962 |
| GSGSPHSKAQGSL | 1963 |
| GSGSPHSKSQNQQ | 1964 |
| GNGSPHSKSQNQQ | 1965 |
| GSGSPHSKAQVPA | 1966 |
| GNGSPHSKAQNLQ | 1967 |
| GSGSPHSKAQDKQ | 1968 |
| GSGSPHSKAHYQQ | 1969 |
| GSGSPHSKAQVPS | 1970 |
| GGGSPHSKAQNHQ | 1971 |
| GSGSPHSKARGEQ | 1972 |
| GGGSPHSKAQYQH | 1973 |
| GSGSPHSKAPGQQ | 1974 |
| KNGSPHSKAQNQQ | 1975 |
| GSGSPHSKRLEQQ | 1976 |
| GSGSPHSKAQNQS | 1977 |
| GSGSPHSKAQKVI | 1978 |
| GSGSPHSKAQNND | 1979 |
| GSGSPHSKAQSVH | 1980 |
| GSGSPHSKAQPLG | 1981 |
| KEGSPHSKAQNQQ | 1982 |
| GSGSPHSKAHNQE | 1983 |
| GSGSPHSKAQIQQ | 1984 |
| GSGSPHSKAQVRN | 1985 |
| GSGSPHSKAPSNQ | 1986 |
| GSGSPHSKAQVGH | 1987 |
| GSGSPHSKAQRDI | 1988 |
| GSGSPHSKAQMPN | 1989 |
| AIGSPHSKAQNQQ | 1990 |
| GSGSPHSKARGLQ | 1991 |
| GSGSPHSKLQKQQ | 1992 |
| GSGSPHSKAPSLQ | 1993 |
| GSGSPHSKAQRDQ | 1994 |
| GSGSPHSKNRDQQ | 1995 |
| GSGSPHSKAQAKG | 1996 |
| GSGSPHSKAQSAH | 1997 |
| GNGSPHSKSQNQH | 1998 |
| GSGSPHSKSQNHQ | 1999 |
| RSGSPHSKAQDQQ | 2000 |
| GSGSPHSKAQSTM | 2001 |
| GSGSPHSKAQREM | 2002 |
| GGGSPHSKSQNRQ | 2003 |
| GSGSPHSKAQYRA | 2004 |
| GGGSPHSKAQRQQ | 2005 |
| GSGSPHSKNQWQQ | 2006 |
| GSGSPHSKAQRMN | 2007 |
| GSGSPHAKAQNHQ | 2008 |
| GSGSPHSKAGDSQ | 2009 |
| GSGSPHSKLKSQQ | 2010 |
| GSGSPHSKAQKIS | 2011 |
| GSGSPHSKAPSMQ | 2012 |
| GSGSPHSKASPRQ | 2013 |
| GSGSPHSKRMEQQ | 2014 |
| GSGSPHSKAQYQN | 2015 |
| GSGSPHARMQNQQ | 2016 |
| GSGSPHVKSQNQQ | 2017 |
| GQDSPHKSGQNQQ | 2018 |
| GSGSPHASRQNQQ | 2019 |
| GSGSPHASRQNKQ | 2020 |
| GSGSPHVKIQNQQ | 2021 |
| GSGSPHSKAKNQQ | 2022 |
| GSGSPHKKNQNQQ | 2023 |
| GSGSPHVRMQNQQ | 2024 |
| GSGSPHASRQKQQ | 2025 |
| GHSSPHRSGQNQQ | 2026 |
| GSGSPHTRGQNQQ | 2027 |
| CSGSPHSKAQNQQ | 2028 |
| GSGSPHRKAQNQQ | 2029 |
| GSGSPHSKAQNKQ | 2030 |
| GSGSPHSKAQTQQ | 2031 |
| CSGSPHKTSQNQQ | 2032 |
| CSHSPHKSGQNQQ | 2033 |
| GQSSPHRSGQNQQ | 2034 |
| GRGSPHASRQNQQ | 2035 |
| GSGSPHASRKNQQ | 2036 |
| GSGSPHASRQNQH | 2037 |
| GSGSPHKFGKNQQ | 2038 |
| GSGSPHKFGQKQQ | 2039 |
| GSGSPHKFGQNQQ | 2040 |
| GSGSPHKIGQNQQ | 2041 |
| GSGSPHKLGQNQQ | 2042 |
| GSGSPHKTSKNQQ | 2043 |
| GSGSPHKTSQKQQ | 2044 |
| GSGSPHKTSQNQQ | 2045 |
| GSGSPHKTTQNQQ | 2046 |
| GSGSPHKTYQNQQ | 2047 |
| GSGSPHKYGQNQQ | 2048 |
| GSGSPHSKAQHQQ | 2049 |
| GSGSPHSKDQNQQ | 2050 |
| GSGSPHSKPQNQQ | 2051 |
| GSGSPHVRGQKQQ | 2052 |
| GSGSPHVRGQNKQ | 2053 |
| GSHSPHKRGQNQQ | 2054 |
| GSHSPHKSGQKQQ | 2055 |
| GSHSPHKSGQNQQ | 2056 |
| VSGSPHASRQNQQ | 2057 |
| VSGSPHGARQNQQ | 2058 |
| VSGSPHKFGQNQQ | 2059 |
| GSGSPHSKAQYYV | 2060 |
| GSGSPHSKLRRQQ | 2061 |
| GSGSPHSKAGCGQ | 2062 |
| GSGSPHSRAQNQQ | 2063 |
| GSGSPHSKRLRQQ | 2064 |
| GSGSPHSLRRNQQ | 2065 |
| GSGSPHSRGRNQQ | 2066 |
| GSGSPHSSRRNQQ | 2067 |
| GSGSPHSKAFRLQ | 2068 |
| GSCSPHRKAQNQQ | 2069 |
| GSGSPHFLRQNQQ | 2070 |
| GSGSPHSLRENQQ | 2071 |
| GSGSPHSKWLLQQ | 2072 |
| GSGSPHSKRRLQQ | 2073 |
| GSGSPHSKAQRKL | 2074 |
| GSGSPHSKALRRQ | 2075 |
| GSGSPHSKAQRLR | 2076 |
| GSGSPHSKAQRRL | 2077 |
| GSGSPHSKARRQQ | 2078 |
| GSGSPHSKARRLQ | 2079 |
| GSGSPHSKSRRQQ | 2080 |
| GLLSPHWKAQNQQ | 2081 |
| GSGSPHSKARLRQ | 2082 |
| GSGSPHSKASKRQ | 2083 |
| GSGSPHVRRQNQQ | 2084 |
| GSGSPHSKAQLYR | 2085 |
| GSGSPHSKAQLTV | 2086 |
| GHDSPHKRGQHRQ | 2087 |
| GHDSPHKSGQKQQ | 2088 |
| GHDSPHKSGLTQQ | 2089 |
| GDDSPHKSGRNQQ | 2090 |
| GHDSPHKSGLNQQ | 2091 |
| GHESPHKSAQNHQ | 2092 |
| GHDSPHKSAQNQW | 2093 |
| GHDSPHKSGQNTH | 2094 |
| GHDSPHKSGRRRQ | 2095 |
| GHDSPHKSAQNQQ | 2096 |
| GHDSPHKSGQYQQ | 2097 |
| GNYSPHKIGQNQQ | 2098 |
| GHDSPHKSRQNDQ | 2099 |
| GHDSPHKSGQIRQ | 2100 |
| GHDSPHKIGQNQH | 2101 |
| GYDSPHKSGQKQQ | 2102 |
| GHDSPHKSGQSVQ | 2103 |
| GHESPRKSGRSQQ | 2104 |
| GHDSPHKSGQNKL | 2105 |
| GHDSPHKTGQNQQ | 2106 |
| GRGSPHKRGQNQQ | 2107 |
| GSGSPHTKAQNPP | 2108 |
| GQDSPHKSGQHQQ | 2109 |
| GHDSPHKSGQIQH | 2110 |
| GHDSPHKSGPRQQ | 2111 |
| GHDSPHKSGHTQQ | 2112 |
| GHDSPHKSGQRQH | 2113 |
| GSGSPHTKAQNQQ | 2114 |
| GHDSPHKSAQSQQ | 2115 |
| GHESPRKSGQNQQ | 2116 |
| GHDSPHKSLQNQQ | 2117 |
| GHGSPHSKAQNPQ | 2118 |
| GHDSPHKSGRNQE | 2119 |
| GHDSPHKSGQTQL | 2120 |
| GHDSPHKSEKNQQ | 2121 |
| GRDSPHKSGQDQQ | 2122 |
| GHDSPHKTGHNQQ | 2123 |
| GYDSPHKSGQTQQ | 2124 |
| GHESPHKSGQTQQ | 2125 |
| GHDSPHKSGQSKQ | 2126 |
| GHDSPHKTGQNQP | 2127 |
| GHDSPHKSGQSPQ | 2128 |
| GNDSPHKSVQNQQ | 2129 |
| GHDSPHKSAQNYQ | 2130 |
| GSASPHSKALNQQ | 2131 |
| GHESPHKSAQNRQ | 2132 |
| GQDSPHKIGQNQQ | 2133 |
| GHDSPHKSGQNHL | 2134 |
| GHDSPHKSGQYQH | 2135 |
| GNDSPHKSVQNHQ | 2136 |
| GHDSPHKSGQNQW | 2137 |
| GHDSPHKSVQNQH | 2138 |
| GHDSPHKSGQNQH | 2139 |
| GHDSPHKSGQTRQ | 2140 |
| GHDSPHKSGQNLH | 2141 |
| GHDSPHKSAQNQE | 2142 |
| GHDSPHKSGQHLQ | 2143 |
| GHDSPHKSRLNQP | 2144 |
| GQDSPHKSGQNQD | 2145 |
| GHDSPHKSGRNQQ | 2146 |
| GHDSPHKSGQNLL | 2147 |
| GHDSPHKSGQLVI | 2148 |
| GHDSPHKSRQSQQ | 2149 |
| GHDSPHKSGRTQE | 2150 |
| GHDSPHKSVQTHQ | 2151 |
| GHDSPHKSGQNQP | 2152 |
| GHDSPHKSGQTQQ | 2153 |
| GPDSPHKIGQNQQ | 2154 |
| GHDSPHKSVQNQQ | 2155 |
| GHDSPHKSRQDQH | 2156 |
| GPDSPHKSGQKQQ | 2157 |
| GHDSPHKSRQSQH | 2158 |
| GHDSPHKSVQNQL | 2159 |
| GYDSPHKSGQYQH | 2160 |
| GHDSPHKSRQNQQ | 2161 |
| GHDSPHKSWVRQQ | 2162 |
| GHESPHKSGQNQH | 2163 |
| GHDSPHKIGHNQQ | 2164 |
| GHDSPHKSNAWQQ | 2165 |
| GHDSPHKSGQSVP | 2166 |
| GHESPHKSGQNIQ | 2167 |
| GHDSPHKSVQNHL | 2168 |
| GHDSPHKIGLDQQ | 2169 |
| ASGSPHSKAQHQQ | 2170 |
| GHDSPHKRGPDQQ | 2171 |
| GMGSPHSKTQNQQ | 2172 |
| GHDSPHKSGESQQ | 2173 |
| GHDSPHKHGQNHQ | 2174 |
| GTGSPHSKAQNQL | 2175 |
| GHDSPHKSVQNKQ | 2176 |
| GQVSPHKSGQNQQ | 2177 |
| GHDSPHKSGQRQL | 2178 |
| GHDSPHKIGQNQL | 2179 |
| GHDSPHKSGQIIV | 2180 |
| GYDSPHKSGQKQS | 2181 |
| GNGSPHSKAQNQE | 2182 |
| GDDSPHKSVQNQQ | 2183 |
| GHDSPHKSVQSHQ | 2184 |
| GHDSPHKSGQFVV | 2185 |
| GHDSPHKSRQNLQ | 2186 |
| GHNSPHKSGQNQE | 2187 |
| GHDSPHKSGQSQP | 2188 |
| GHESPHKSGQNEQ | 2189 |
| GHDSPHKSGQNQL | 2190 |
| GHDSPHKSAQNLL | 2191 |
| ASGSPHSKAINQQ | 2192 |
| GNGSPHKRGQNQQ | 2193 |
| GHDSPHKSGQSLQ | 2194 |
| GHDSPHKSAQNHQ | 2195 |
| GHDSPHKSGRNRQ | 2196 |
| GHDSPHKYGQNEQ | 2197 |
| GNGSPHSKAPNLQ | 2198 |
| GHDSPHKSQQNQQ | 2199 |
| GHDSPHKSVQSKQ | 2200 |
| GNDSPHKIGHNQQ | 2201 |
| GGGSPHSKAQDQQ | 2202 |
| GQDSPHKSGQNPL | 2203 |
| ASGSPHSKAQNHQ | 2204 |
| GHDSPHKSGRDQK | 2205 |
| GHDSPHKSVHNQQ | 2206 |
| GHDSPHKSGQWKR | 2207 |
| GSGSPHSKAENRQ | 2208 |
| GHDSPHKSGQSQQ | 2209 |
| GHDSPHKSRQAQQ | 2210 |
| GHDSPHKSVQNHQ | 2211 |
| GHDSPHKSKQNQQ | 2212 |
| GHDSPHKSAQNQL | 2213 |
| GHDSPHKSGQTQP | 2214 |
| GHDSPHKLWINQQ | 2215 |
| GPDSPHKSGQNQQ | 2216 |
| GHDSPHKSVQKQL | 2217 |
| GHPSPHWKGQNQQ | 2218 |
| GHDSPHKSGRNQL | 2219 |
| GSGSPHSKVQDQQ | 2220 |
| GHDSPHKMGRNQQ | 2221 |
| GHDSPHKSGISIQ | 2222 |
| GHDSPHKSVQNLQ | 2223 |
| GHDSPHKMAHNQQ | 2224 |
| GHDSPHKHGQNQQ | 2225 |
| GHDSPHKSVQSQQ | 2226 |
| GHDSPHKSGQTVC | 2227 |
| GQDSPHKSGQYQQ | 2228 |
| GHDSPHKSGQQIM | 2229 |
| GHDSPHKSRQNEQ | 2230 |
| GHDSPHKSGLNHQ | 2231 |
| GYDSPHKSGQNQQ | 2232 |
| GHDSPHKSGQNLQ | 2233 |
| GHDSPHKSRQDQQ | 2234 |
| GDDSPHKSGQKQL | 2235 |
| GSGSPHSKAQNQA | 2236 |
| GDDSPHKSGHNQQ | 2237 |
| GHDSPHKSGQMIH | 2238 |
| GHDSPHKSGRNHQ | 2239 |
| GHDSPHKSVQNRQ | 2240 |
| GHDSPHKSGQKMN | 2241 |
| TINGSGSPHSKAQNQQ | 2242 |
| TINGHDSPHKSGQNQQ | 2243 |
| TIIGSGSPHSKAQNRH | 2244 |
| TFPGSGSPHSKVQNQQ | 2245 |
| TEKMSGSPHSKAQNQQ | 2246 |
| EINGRGSPHSKAQNQQ | 2247 |
| TVNRNGSPHSKAQNQQ | 2248 |
| TVNGSGSPHSKARDQQ | 2249 |
| TFNGSGSPHSKAPNLQ | 2250 |
| TEKTSGSPHSKAQNQQ | 2251 |
| TINGSGSPHSKAHVRQ | 2252 |
| TVNGSGSPHSKAPNQH | 2253 |
| TEKISGSPHSKAQNQQ | 2254 |
| TINGPGSPHSKAHNQQ | 2255 |
| TVNGSGSPHSKTQSQQ | 2256 |
| SINESGSPHSKAQNQQ | 2257 |
| TERTSGSPHSKAQNQQ | 2258 |
| TINGSGSPHSKAQPAK | 2259 |
| TEKSSGSPHSKAQNQQ | 2260 |
| TSYGNGSPHSKAQNQQ | 2261 |
| TEKGSGSPHSKAQNQQ | 2262 |
| TINGSGSPHSKSQTQQ | 2263 |
| TERISGSPHSKAQNQQ | 2264 |
| TERASGSPHSKAQNQQ | 2265 |
| ELHGSGSPHSKAQNQQ | 2266 |
| AINGSGSPHSKAQNLA | 2267 |
| TVNGSGSPHSKSQNQL | 2268 |
| TERNSGSPHSKAQNQQ | 2269 |
| SVNGNGSPHSKAQNQQ | 2270 |
| TFNGSGSPHSKAQGQQ | 2271 |
| TERVSGSPHSKAQNQQ | 2272 |
| TINGSGSPHSKALNRQ | 2273 |
| TERLSGSPHSKAQNQQ | 2274 |
| TDNGSGSPHSKAHNQQ | 2275 |
| TFHGSGSPHSKTQNQQ | 2276 |
| TINGGGSPHSKAQTQQ | 2277 |
| TSNGSGSPHSKAQNPP | 2278 |
| TINGSGSPHSKAQNLQ | 2279 |
| TVHGNGSPHSKAQNQQ | 2280 |
| TINGGGSPHSKAQNQQ | 2281 |
| TENMSGSPHSKAQNQQ | 2282 |
| TENVSGSPHSKAQNQQ | 2283 |
| TSSGSGSPHSKAQYQQ | 2284 |
| TIDGGGSPHSKAQNKQ | 2285 |
| TEKVSGSPHSKAQNQQ | 2286 |
| AINGSGSPHSKAQDQE | 2287 |
| TQNKSGSPHSKAQNQQ | 2288 |
| TINGGGSPHSKAQNQL | 2289 |
| NINGGGSPHSKAQNQQ | 2290 |
| TEHLSGSPHSKAQNQQ | 2291 |
| AEMGSGSPHSKAQNQQ | 2292 |
| ATNGSGSPHSKAQNHQ | 2293 |
| AIKGSGSPHSKAQDQQ | 2294 |
| TINGGGSPHSKSQNQL | 2295 |
| TVNGNGSPHSKAQNKQ | 2296 |
| TINGSGSPHSKGHWQQ | 2297 |
| TDKTSGSPHSKAQNQQ | 2298 |
| TFKGSGSPHSKAPNQQ | 2299 |
| TVNGSGSPHSKAQNQL | 2300 |
| TINGSGSPHSKRPEQQ | 2301 |
| TINGSGSPHSKAQRTM | 2302 |
| TEKASGSPHSKAQNQQ | 2303 |
| SDQGSGSPHSKAQNQQ | 2304 |
| TEITSGSPHSKAQNQQ | 2305 |
| TDKSSGSPHSKAQNQQ | 2306 |
| TIDGSGSPHSKAQNQQ | 2307 |
| TVNGNGSPHSKAQNQH | 2308 |
| NINGSGSPHSKAQNQQ | 2309 |
| TETHSGSPHSKAQNQQ | 2310 |
| TINGGGSPHSKALNQQ | 2311 |
| TINGSGSPHSKALHQH | 2312 |
| TINGTGSPHSKAQNHQ | 2313 |
| TINGSGSPHSKAQHRI | 2314 |
| TINGSGSPHSKAQYIH | 2315 |
| TENISGSPHSKAQNQQ | 2316 |
| THIGGGSPHSKAHNQQ | 2317 |
| TINGSGSPHSKAQKFE | 2318 |
| TSNESGSPHSKAQNHQ | 2319 |
| TINGSGSPHSKAQFPS | 2320 |
| TERPSGSPHSKAQNQQ | 2321 |
| TINGNGSPHSKAQNPL | 2322 |
| SIKGNGSPHSKAQNQQ | 2323 |
| TERMSGSPHSKAQNQQ | 2324 |
| TERSSGSPHSKAQNQQ | 2325 |
| TELHSGSPHSKAQNQQ | 2326 |
| TELTSGSPHSKAQNQQ | 2327 |
| TINGSGSPHSKAHNQQ | 2328 |
| TINGGGSPHSKAQSQQ | 2329 |
| TINGSGSPHSKAQAIK | 2330 |
| TENTSGSPHSKAQNQQ | 2331 |
| TIDGSGSPHSKGQNRQ | 2332 |
| NINGSGSPHSKAQSQQ | 2333 |
| TINGSVSPHGKAQNQL | 2334 |
| TSNASGSPHSKAQNQL | 2335 |
| TEARSGSPHSKAQNQQ | 2336 |
| TEKNSGSPHSKAQNQQ | 2337 |
| TANGSGSPHSKAQYQQ | 2338 |
| TVNGSGSPHSKAQYQH | 2339 |
| TINGSGSPHTKAQNPQ | 2340 |
| TINGSGSPHSKGQNPP | 2341 |
| TIIGSGSPHSKAQHQL | 2342 |
| TINGSGSPHSKAQSPP | 2343 |
| TIYGSGSPHSKAQNQQ | 2344 |
| TINGSGSPHSKAQAKL | 2345 |
| TDKNSGSPHSKAQNQQ | 2346 |
| TINGSGSPHSKTKSQQ | 2347 |
| TINGSGSPHSKAQDRP | 2348 |
| TINGIGSPHSKAQNLG | 2349 |
| TINGSGSPHSKAQSQQ | 2350 |
| TENLSGSPHSKAQNQQ | 2351 |
| TINGSGSPHSKAQAFH | 2352 |
| TINGSGSPHSKAQKQQ | 2353 |
| TFSGSGSPHSKAQNLQ | 2354 |
| AINGSGSPHSKAQNAQ | 2355 |
| TESWSGSPHSKAQNQQ | 2356 |
| TINGSGSPHSKAHNQL | 2357 |
| TVNGNGSPHSKAQNHQ | 2358 |
| TEDKSGSPHSKAQNQQ | 2359 |
| TESASGSPHSKAQNQQ | 2360 |
| TNNGSGSPHSKAQNQQ | 2361 |
| TSNGGGSPHSKAQNLQ | 2362 |
| TDKMSGSPHSKAQNQQ | 2363 |
| EVHGSGSPHSKAQNQQ | 2364 |
| TINGSGSPHSKAQKLN | 2365 |
| TINGGGSPHSKSQNQH | 2366 |
| TVNGGGSPHSKAQSQQ | 2367 |
| TINGSGSPHSKAQYQH | 2368 |
| TISGSGSPHSKAQYQH | 2369 |
| TESTSGSPHSKAQNQQ | 2370 |
| TINGSGSPHSKSQNVQ | 2371 |
| SINGSGSPHSKAQAQQ | 2372 |
| TVNGSGSPHSKAQNLQ | 2373 |
| TVRDSGSPHSKAQNQQ | 2374 |
| TFNASGSPHSKAPNQQ | 2375 |
| TDRMSGSPHSKAQNQQ | 2376 |
| TINGSGSPHSKAQTPP | 2377 |
| TIKGSGSPHSKAQNQQ | 2378 |
| NHIGSGSPHSKAQNQQ | 2379 |
| TINGSGSPHSKAQYQH | 2380 |
| TIPIDGSPHSKAQNQQ | 2381 |
| TINGSGSPHSKAQGQQ | 2382 |
| TFNGSGSPHNKAQNHQ | 2383 |
| ESDGSGSPHSKAQNQQ | 2384 |
| TINGSGSPHSKAQPPA | 2385 |
| TINGSGSPHSKAQERP | 2386 |
| TIKGSGSPHSKAQDLQ | 2387 |
| TDLKSGSPHSKAQNQQ | 2388 |
| TINGGGSPHSKAQNPP | 2389 |
| TINGSGSPHSKAQAMH | 2390 |
| TVPNSGSPHSKAQNQQ | 2391 |
| TVIGSGSPHSKALNQQ | 2392 |
| TINGSGSPHSKAQHPS | 2393 |
| TINGLGSPHSKSQNQQ | 2394 |
| TINGTGSPHSKAQNQQ | 2395 |
| TINGSGSPHSKAPGLQ | 2396 |
| TINGSGSPHSKAQGIR | 2397 |
| TESHSGSPHSKAQNQQ | 2398 |
| TINGSGSPHSKAQAPA | 2399 |
| TINGSGSPHSKSQSQQ | 2400 |
| AEHGSGSPHSKAQNQQ | 2401 |
| TEDRSGSPHSKAQNQQ | 2402 |
| NCLGSGSPHSKAQNQQ | 2403 |
| TDRGSGSPHSKAQNQQ | 2404 |
| TINGSGSPHSKAQIPP | 2405 |
| TVKGSGSPHSKAQDQQ | 2406 |
| NADGSGSPHSKAQNQQ | 2407 |
| TDKVSGSPHSKAQNQQ | 2408 |
| TITGSGSPHSKAQTQL | 2409 |
| TINGSGSPHSKAQAPS | 2410 |
| NCVGSGSPHSKAQNQQ | 2411 |
| TIRDAGSPHSKAQNQQ | 2412 |
| TVKDSGSPHSKAQNQQ | 2413 |
| NALGSGSPHSKAQNQQ | 2414 |
| VINGSGSPHSKGQNQQ | 2415 |
| TVNGGGSPHSKAQNQQ | 2416 |
| TIQDGGSPHSKAQNQQ | 2417 |
| TISGGGSPHSKAQNQQ | 2418 |
| TSNASGSPHSKAHNQQ | 2419 |
| TINGSGSPHSKAQNTY | 2420 |
| TINGSGSPHSKSQNQH | 2421 |
| TEFVSGSPHSKAQNQQ | 2423 |
| TVNGSGSPHSKAQNHL | 2424 |
| TREISGSPHSKAQNQQ | 2425 |
| TINGSGSPHSKAQIGM | 2426 |
| TIDGSGSPHSKALNKQ | 2427 |
| TIIGGGSPHSKAQNPQ | 2428 |
| QGEGSGSPHSKAQNQQ | 2429 |
| TINGTGSPHSKAPNQL | 2430 |
| TVNGSGSPHSKAQLQQ | 2431 |
| TFNGGGSPHSKAQYQQ | 2432 |
| SINGSGSPHSKTQSQQ | 2433 |
| TVNGGGSPHSKAQHQQ | 2434 |
| SEKGSGSPHSKAQNQQ | 2435 |
| NVNGSGSPHSKAQNQQ | 2436 |
| GGEGSGSPHSKAQNQQ | 2437 |
| TINGSGSPHSKAQRMS | 2438 |
| TINGSGSPHSKAQGIL | 2439 |
| EFVGSGSPHSKAQNQQ | 2440 |
| TIIGSGSPHSKAQDRQ | 2441 |
| SDKGSGSPHSKAQNQQ | 2442 |
| TEQVSGSPHSKAQNQQ | 2443 |
| TEHVSGSPHSKAQNQQ | 2444 |
| TINGSGSPHSKARDWQ | 2445 |
| TENASGSPHSKAQNQQ | 2446 |
| EVQGSGSPHSKAQNQQ | 2447 |
| TINGSGSPHSKAQNTH | 2448 |
| TINGSGSPRSKAPNLQ | 2449 |
| TINGSGSPHSKAQERS | 2450 |
| TSNGSGSPHSKAQNYQ | 2451 |
| TEYISGSPHSKAQNQQ | 2452 |
| TINGSGSPHSKAQRTC | 2453 |
| TINGSGSPHSKAQIGH | 2454 |
| NCWGSGSPHSKAQNQQ | 2455 |
| TINGSGSPHSKAQGAI | 2456 |
| TDVNSGSPHSKAQNQQ | 2457 |
| SDIGSGSPHSKAQNQQ | 2458 |
| TINGSGSPHSKAQVPP | 2459 |
| TINGSGSPHSKAQVQQ | 2460 |
| TINGSGSPHSKALMRQ | 2461 |
| TINGSGSPRSKAQYSV | 2462 |
| NSIGSGSPHSKAQNQQ | 2463 |
| TINGSGSPHSKVPNLQ | 2464 |
| AINGSGSPHSKAQSQQ | 2465 |
| TINGSGSPHSKAQAIT | 2466 |
| TINGSGSPHSKAQKTL | 2467 |
| TVNGSGSPHSKAQNQW | 2468 |
| TINGSGSPHSKAQLHH | 2469 |
| TEQTSGSPHSKAQNQQ | 2470 |
| TINGSGSPHSKAQNII | 2471 |
| NSLGSGSPHSKAQNQQ | 2472 |
| TIPMEGSPHSKAQNQQ | 2473 |
| TINGSGSPHSKAQGHH | 2474 |
| TDRTSGSPHSKAQNQQ | 2475 |
| TINGSGSPHSKAQSKV | 2476 |
| EVVGSGSPHSKAQNQQ | 2477 |
| TINGSGSPHSKAQLPS | 2478 |
| TINGSGSPHSKAIGKQ | 2479 |
| TEPTSGSPHSKAQNQQ | 2480 |
| TVNGGGSPHSKSQNQQ | 2481 |
| TINGSGSPHSKAQAIH | 2482 |
| TINGSGSPHSKAQHGL | 2483 |
| SELGSGSPHSKAQNQQ | 2484 |
| TINGSGSPHSKAQFMC | 2485 |
| TINVSGSPHSKAQGQQ | 2486 |
| TINGGGSPHSKAQNQM | 2487 |
| TVNGSGSPHSKAQHLQ | 2488 |
| TIRENGSPRSKAQNQQ | 2489 |
| TINGSGSPHSKTQNHQ | 2490 |
| TINGSGSPHSKAQPAR | 2491 |
| TVNGSGSPHSKAQSLQ | 2492 |
| TINGSGSPHSKSQSQL | 2493 |
| TINGSASPHSKAHSQQ | 2494 |
| TWQNSGSPHSKAQNQQ | 2495 |
| TINGSGSPHSKAQDRQ | 2496 |
| TINGSGSPHSKAQMPS | 2497 |
| TNNGGGSPHSKAQNLQ | 2498 |
| TINGSGSPHSKAQGSL | 2499 |
| TEVTSGSPHSKAQNQQ | 2500 |
| SINGGGSPHSKAQYQQ | 2501 |
| TVIGSGSPHSKSQNQQ | 2502 |
| AVNVSGSPHSKAQNQQ | 2503 |
| TVNGNGSPHSKSQNQQ | 2504 |
| TDRNSGSPHSKAQNQQ | 2505 |
| TINGSGSPHSKAQVPA | 2506 |
| GVLGSGSPHSKAQNQQ | 2507 |
| TLNGNGSPHSKAQNLQ | 2508 |
| AINGSGSPHSKAQDKQ | 2509 |
| TSNGSGSPHSKAHYQQ | 2510 |
| TINGSGSPHSKAQVPS | 2511 |
| TELRSGSPHSKAQNQQ | 2512 |
| NINGSGSPHSKAQNHQ | 2513 |
| TVNGGGSPHSKAQNHQ | 2514 |
| TINGSGSPHSKARGEQ | 2515 |
| TINGGGSPHSKAQYQH | 2516 |
| TEDLSGSPHSKAQNQQ | 2517 |
| TINGSGSPHSKAPGQQ | 2518 |
| TIPKNGSPHSKAQNQQ | 2519 |
| TINGSGSPHSKAQSLQ | 2520 |
| TINGSGSPRSKRLEQQ | 2521 |
| TERGSGSPHSKAQNQQ | 2522 |
| TVNGSGSPHSKAPNQQ | 2523 |
| TSNGSGSPHSKAQNQS | 2524 |
| TINGSGSPHSKAQKVI | 2525 |
| TEGISGSPHSKAQNQQ | 2526 |
| TINGSGSPHSKAQNND | 2527 |
| TINGSGSPHSKAQSVH | 2528 |
| TINGSGSPHSKAQPLG | 2529 |
| TINKEGSPHSKAQNQQ | 2530 |
| TCNASGSPHSKAQNQQ | 2531 |
| AINGSGSPHSKAHNQE | 2532 |
| TEGLSGSPHSKAQNQQ | 2533 |
| TRDASGSPHSKAQNQQ | 2534 |
| TSNGSGSPHSKAQNLQ | 2535 |
| IGNGSGSPHSKAQIQQ | 2536 |
| TVNGGGSPHSKAQNLQ | 2537 |
| TDRSSGSPHSKAQNQQ | 2538 |
| TINGSGSPHSKAQVRN | 2539 |
| TINGSGSPHSKAPSNQ | 2540 |
| TINGSGSPHSKAQVGH | 2541 |
| NAIGSGSPHSKAQNQQ | 2542 |
| AENGSGSPHSKAQNQQ | 2543 |
| TINGSGSPHSKAQRDI | 2544 |
| TINGSGSPHSKAQMPN | 2545 |
| TVNGSGSPHSKSQNQQ | 2546 |
| TIPAIGSPHSKAQNQQ | 2547 |
| TINGSGSPHSKARGLQ | 2548 |
| TELGSGSPHSKAQNQQ | 2549 |
| AETGSGSPHSKAQNQQ | 2550 |
| TINGSGSPHSKLQKQQ | 2551 |
| TINGSGSPHSKAPSLQ | 2552 |
| TINGSGSPHSKAQRDQ | 2553 |
| TDVGSGSPHSKAQNQQ | 2554 |
| TINGSGSPHSKNRDQQ | 2555 |
| SINGSGSPHSKAPNLQ | 2556 |
| TINGSGSPHSKAQAKG | 2557 |
| TVNGSGSPHSKAQDKQ | 2558 |
| TINGGGSPHSKAQNPQ | 2559 |
| TINGSGSPHSKAQSAH | 2560 |
| TINGNGSPHSKSQNQH | 2561 |
| TVPTSGSPHSKAQNQQ | 2562 |
| TIDGSGSPHSKSQNHQ | 2563 |
| TDVKSGSPHSKAQNQQ | 2564 |
| AINRSGSPHSKAQDQQ | 2565 |
| TINGSGSPHSKAQSTM | 2566 |
| TVNASGSPHSKAQNQL | 2567 |
| TINGSGSPHSKAQREM | 2568 |
| TVHGSGSPHSKAQSQQ | 2569 |
| TINGGGSPHSKSQNRQ | 2570 |
| TINGSGSPHSKAQYRA | 2571 |
| TINGGGSPHSKAQRQQ | 2572 |
| TEPMSGSPHSKAQNQQ | 2573 |
| TINGSGSPHSKNQWQQ | 2574 |
| ETAGSGSPHSKAQNQQ | 2575 |
| TINGSGSPHSKAQRMN | 2576 |
| NNLGSGSPHSKAQNQQ | 2577 |
| TINGSGSPHAKAQNHQ | 2578 |
| TIKNGSPHSKAQNQQ | 2579 |
| TINGSGSPHSKAGDSQ | 2580 |
| TINGSGSPHSKLKSQQ | 2581 |
| TINGSGSPHSKAQKIS | 2582 |
| TEYNSGSPHSKAQNQQ | 2583 |
| TINGSGSPHSKAPSMQ | 2584 |
| AINGSGSPHSKAQNQQ | 2585 |
| TINGSGSPHSKASPRQ | 2586 |
| TINGSGSPHSKRMEQQ | 2587 |
| TINGSGSPHSKAQYQN | 2588 |
| TINGSGSPHSKAQYYV | 2589 |
| TINGSGSPHSKLRRQQ | 2590 |
| TINGSGSPHSKAGQGQ | 2591 |
| SMNGSGSPHSRAQNQQ | 2592 |
| TINGSGSPHSKRLRQQ | 2593 |
| TINGSGSPHSLRRNQQ | 2594 |
| TINGSGSPHSRGRNQQ | 2595 |
| TINGSGSPHSSRRNQQ | 2596 |
| TINGSGSPHSKAFRLQ | 2597 |
| TINGSCSPHRKAQNQQ | 2598 |
| TINGSGSPHFLRQNQQ | 2599 |
| TINGSGSPHSLRFNQQ | 2600 |
| TINGSGSPHSKWLLQQ | 2601 |
| TINGSGSPHSKRRLQQ | 2602 |
| TINGSGSPHSKAQRKL | 2603 |
| TINGSGSPHSKALRRQ | 2604 |
| TINGSGSPHSKAQRLR | 2605 |
| YLSGSGSPHSKAQNQQ | 2606 |
| TINGSGSPHSKAQRRL | 2607 |
| TINGSGSPHSKARRQQ | 2608 |
| TINGSGSPHSKARRLQ | 2609 |
| TINGSGSPHSKSRRQQ | 2610 |
| TINGLLSPRWKAQNQQ | 2611 |
| TINGSGSPHSKARLRQ | 2612 |
| TINGSGSPHSKASKRQ | 2613 |
| TINGSGSPHVRRQNQQ | 2614 |
| TINGSGSPHSKAQLYR | 2615 |
| GLSGSGSPHSKAQNQQ | 2616 |
| TINGSGSPHSKAQLTV | 2617 |
| TINGHDSPHKRGQHRQ | 2618 |
| MPEGHDSPHKSGQNQQ | 2619 |
| MEGGHDSPHKSGQNQQ | 2620 |
| MEYGHQSPAKSGQNQQ | 2621 |
| AEWGHDSPHKSGQNQQ | 2622 |
| CEWGHDSPAKSGQNQQ | 2623 |
| ANNGQDSPHKSCQNQQ | 2624 |
| JPEGHDSPHKSGQNQQ | 2625 |
| ADMGHDSPHKSCQNQQ | 2626 |
| IEYGRDSPHKSGQNQQ | 2627 |
| ADYGHDSPHKSGQNQQ | 2628 |
| IETGHDSPHKSGQNQQ | 2629 |
| MEWGHDSPHKSGQNQQ | 2630 |
| CEYGHDSPHKSGQNQQ | 2631 |
| RINGHDSPHKSGQKQQ | 2632 |
| MEIGHDSPHKSGQNQQ | 2633 |
| LEYGHDSPHKSGQNQQ | 2634 |
| ADWGHDSPHKSGQNQQ | 2635 |
| IEIGHDSPHKSGQNQQ | 2636 |
| DIMGHDSPHKSGQNQQ | 2637 |
| FEQGHDSPHKSGQNQQ | 2638 |
| MEFGHDSPHKSGQNQQ | 2639 |
| CDQGHDSPHKSGQNQQ | 2640 |
| LPEGHDSPHKSGQNQQ | 2641 |
| JENGHDSPHKSGQNQQ | 2642 |
| MESGHDSPHKSGQNQQ | 2643 |
| AEIGHDSPHKSGQNQQ | 2644 |
| VEYGHDSPHKSGQNQQ | 2645 |
| IINGRDSPHKSGLTQQ | 2646 |
| TSNGDDSPHKSGRNQQ | 2647 |
| IEVGRDSPHKSGQNQQ | 2648 |
| MEMGHDSPHKSGQNQQ | 2649 |
| AEVGHDSPHKSGQNQQ | 2650 |
| MDAGHDSPHKSGQNQQ | 2651 |
| VEWGHDSPHKSGQNQQ | 2652 |
| AEQGHDSPHKSGQNQQ | 2653 |
| LEWGHDSPHKSGQNQQ | 2654 |
| MELGHDSPHKSGQNQQ | 2655 |
| METGHDSPHKSGQNQQ | 2656 |
| MEAGHDSPAKSGQNQQ | 2657 |
| IESGHDSPHKSGQNQQ | 2658 |
| MEVGHDSPHKSGQNQQ | 2659 |
| CEIGHDSPHKSGQNQQ | 2660 |
| ATNGHDSPHKSGLNQQ | 2661 |
| MDGGHDSPHKSGQNQQ | 2662 |
| QEVGHDSPHKSGQNQQ | 2663 |
| ADQGHDSPHKSGQNQQ | 2664 |
| TINGHESPHKSAQNHQ | 2665 |
| TINGHDSPHKSAQNQW | 2666 |
| NMNGHDSPHKSGQNTH | 2667 |
| IEMGHDSPHKSGQNQQ | 2668 |
| TINGHDSPHKSGRRRQ | 2669 |
| ISNGHDSPHKSAQNQQ | 2670 |
| TGNGHDSPHKSGQYQQ | 2671 |
| TINGNYSPHKIGQNQQ | 2672 |
| TINGHDSPHKSRQNDQ | 2673 |
| QQQGHDSPHKSGQNQQ | 2674 |
| HDWGHDSPHKSGQNQQ | 2675 |
| IEGGHDSPHKSGQNQQ | 2676 |
| TFNRSGSPHSKAQNQQ | 2677 |
| AINGHDSPHKSGQIRQ | 2678 |
| TINGHDSPHKIGQNQH | 2679 |
| AINGYDSPHKSGQKQQ | 2680 |
| TESGHDSPHKSGQNQQ | 2681 |
| TINGRDSPHKSGQSVQ | 2682 |
| TINGRESPHKSGRSQQ | 2683 |
| TINGRDSPHKSGQNKL | 2684 |
| TINGHDSPHKTGQNQQ | 2685 |
| TINGRGSPHKRGQNQQ | 2686 |
| TINGSGSPHTKAQNPP | 2687 |
| TINGQDSPHKSGQHQQ | 2688 |
| SINGHDSPHKSGQIQH | 2689 |
| AINGHDSPHKSGPRQQ | 2690 |
| TVNGHDSPHKSGHTQQ | 2691 |
| SINGHDSPHKSGQRQH | 2692 |
| SLNGSGSPRTKAQNQQ | 2693 |
| AINGHDSPHKSAQSQQ | 2694 |
| SIYGHESPHKSGQNQQ | 2695 |
| TVNGHDSPHKSLQNQQ | 2696 |
| TINGHGSPHSKAQNPQ | 2697 |
| TSNGYDSPHKSGQKQQ | 2698 |
| TVNGHDSPHKSGRNQE | 2699 |
| TTNGHDSPHKSGQTQL | 2700 |
| AINGHDSPHKSEKNQQ | 2701 |
| IINGRDSPHKSGQDQQ | 2702 |
| TISGHDSPHKTGHNQQ | 2703 |
| SINGYDSPHKSGQTQQ | 2704 |
| TINGHESPHKSGQTQQ | 2705 |
| TINGHDSPHKSGQSKQ | 2706 |
| AHIGHESPHKSGQNQQ | 2707 |
| TINGHDSPHKTGQNQP | 2708 |
| AINGHDSPHKSGQSPQ | 2709 |
| TIKGNDSPHKSVQNQQ | 2710 |
| TEFGHDSPHKSGQNQQ | 2711 |
| TINGHDSPHKSAQNYQ | 2712 |
| TENGSASPHSKALNQQ | 2713 |
| TINGHESPHKSAQNRQ | 2714 |
| TTNGHDSPHKSGQNQQ | 2715 |
| TIKGQDSPHKIGQNQQ | 2716 |
| TVNGHDSPHKSGQNAL | 2717 |
| SINGHDSPHKSGQYQH | 2718 |
| TINGNDSPHKSVQNHQ | 2719 |
| TITGHDSPHKSGQNQW | 2720 |
| TNNGHDSPHKSVQNQH | 2721 |
| TIDGRDSPHKSGQNQH | 2722 |
| TVNGHDSPHKSGQTRQ | 2723 |
| TVNGHDSPHKSGQNLH | 2724 |
| AISGHDSPHKSGLNQQ | 2725 |
| AINGHDSPHKSAQNQE | 2726 |
| TITGHDSPRKSGQHLQ | 2727 |
| TINGHDSPHKSRLNQP | 2728 |
| TVDGHDSPHKSGQKQQ | 2729 |
| TINGQDSPHKSGQNQD | 2730 |
| TIEGHDSPHKSGRNQQ | 2731 |
| TTNGHDSPHKSGQNLL | 2732 |
| TINGHDSPHKSGQLVI | 2733 |
| TVNGHDSPHKSRQSQQ | 2734 |
| TINGHDSPHKSGRTQE | 2735 |
| TINGHDSPHKSVQTHQ | 2736 |
| TSNGHDSPHKSGQNQP | 2737 |
| VINGHDSPHKSGQTQQ | 2738 |
| TINGPDSPHKIGQNQQ | 2739 |
| AVNGHDSPHKSVQNQQ | 2740 |
| TINGHDSPHKSRQDQH | 2741 |
| AINGPDSPHKSGQKQQ | 2742 |
| TINGHDSPHKSRQSQH | 2743 |
| TIYGHDSPHKSVQNQL | 2744 |
| TVNGHDSPHKSGQNLL | 2745 |
| TENKSGSPHSKAQNQQ | 2746 |
| TTNGQDSPHKSGQNQQ | 2747 |
| IDKGSGSPHSKAQNQQ | 2748 |
| TIDGHDSPHKSGRNQQ | 2749 |
| TINGYDSPHKSGQYQH | 2750 |
| IDNGHDSPHKSRQNQQ | 2751 |
| TINGHDSPHKSWVRQQ | 2752 |
| IINGHESPRKSGQNQH | 2753 |
| TVNGHDSPHKIGHNQQ | 2754 |
| TCNGHDSPHKSGRNQQ | 2755 |
| TINGNGSPHSKAQNHQ | 2756 |
| NVVGHDSPHKSGQNQQ | 2757 |
| TINGHDSPHKSNAWQQ | 2758 |
| IDAGHDSPHKSGQNQQ | 2759 |
| TEVGHDSPHKSGQNQQ | 2760 |
| SELGHDSPHKSGQNQQ | 2761 |
| TINGHDSPHKSGQSVE | 2762 |
| TINGHESPHKSGQNIQ | 2763 |
| TINGHDSPHKSVQNHL | 2764 |
| TINGHDSPHKIGLDQQ | 2765 |
| TSNASGSPHSKAQHQQ | 2766 |
| TINGHDSPHKRGPDQQ | 2767 |
| TINGMGSPHSKTQNQQ | 2768 |
| TIKGHDSPHKSGESQQ | 2769 |
| TINGHDSPHKHGQNHQ | 2770 |
| TVNGTGSPHSKAQNQL | 2771 |
| THIGHDSPHKSGQYQH | 2772 |
| TSNGHDSPHKSVQNKQ | 2773 |
| IVNGQVSPAKSGQNQQ | 2774 |
| TVNGHDSPHKSGQRQL | 2775 |
| TVNGHDSPHKIGQNQL | 2776 |
| TINGHDSPHKSGQIIV | 2777 |
| IGNGHESPHKSGQNQQ | 2778 |
| EVMGHDSPHKSGQNQQ | 2779 |
| TINGYDSPHKSGQKQS | 2780 |
| TIHGNGSPHSKAQNQE | 2781 |
| YQVGHDSPHKSGQNQQ | 2782 |
| TIKGDDSPHKSVQNQQ | 2783 |
| TINGHDSPHKSVQSHQ | 2784 |
| TINGHDSPHKSGQFVV | 2785 |
| TVNGHDSPAKSRQNLQ | 2786 |
| ATNGHNSPAKSGQNQE | 2787 |
| AINGHDSPHKSAQNQQ | 2788 |
| TEHGHDSPHKSGQNQQ | 2789 |
| TIYGHDSPHKSGQSQE | 2790 |
| TISGHESPHKSGQNEQ | 2791 |
| AHIGHDSPHKSAQNQQ | 2792 |
| AIDGHDSPHKSGQNQL | 2793 |
| TIMGHDSPHKSVQNQQ | 2794 |
| EVGGHDSPHKSGQNQQ | 2795 |
| TINGHDSPHKSAQNLL | 2796 |
| TINASGSPHSKAINQQ | 2797 |
| AINGNGSPHKRGQNQQ | 2798 |
| SEMGHDSPHKSGQNQQ | 2799 |
| AQQGHDSPHKSGQNQQ | 2800 |
| AINGHDSPHKSGQSLQ | 2801 |
| TINGSGSPHSKAPNQQ | 2802 |
| CGEGHDSPHKSGQNQQ | 2803 |
| TVNGHDSPHKSAQNHQ | 2804 |
| TVNGHDSPHKSGQTQL | 2805 |
| INNGHDSPHKSGRNRQ | 2806 |
| TINGHDSPHKYGQNEQ | 2807 |
| TINGNGSPHSKAPNLQ | 2808 |
| SINGHDSPHKSQQNQQ | 2809 |
| IIGGHDSPHKSGQNQQ | 2810 |
| TINGHDSPHKSVQSKQ | 2811 |
| ELVGHDSPHKSGQNQQ | 2812 |
| ELMGHDSPHKSGQNQQ | 2813 |
| TINGNDSPHKIGHNQQ | 2814 |
| IIKGGGSPHSKAQDQQ | 2815 |
| TVNGHDSPAKSGQTQQ | 2816 |
| TINGQDSPHKSGQNPL | 2817 |
| TVNASGSPHSKAQNHQ | 2818 |
| TINGHDSPHKSGRDQK | 2819 |
| TINGHDSPHKSVHNQQ | 2820 |
| TINGHDSPHKSGQWKR | 2821 |
| TIDGSGSPHSKAENRQ | 2822 |
| NEIGHDSPHKSGQNQQ | 2823 |
| AINGHDSPHKSGQSQQ | 2824 |
| IINGHDSPHKSRQAQQ | 2825 |
| IPNGHDSPHKSGQNQQ | 2826 |
| ITNGHDSPHKSGQTQQ | 2827 |
| TINGHDSPHKSVQNHQ | 2828 |
| TINGHDSPHKSKQNQQ | 2829 |
| TINGHDSPHKSAQNQI | 2830 |
| TVNGHDSPHKSGQTQP | 2831 |
| TDQGHDSPHKSGQNQQ | 2832 |
| TINGHDSPHKLWINQQ | 2833 |
| GINGPDSPHKSGQNQQ | 2834 |
| SEIGHDSPHKSGQNQQ | 2835 |
| TINGHDSPHKSVQKQL | 2836 |
| TINGHPSPHWKGQNQQ | 2837 |
| TVNGHDSPHKSGRNQL | 2838 |
| TIKGSGSPHSKVQDQQ | 2839 |
| SEKGHDSPHKSGQNQQ | 2840 |
| WSAGHDSPHKSGQNQQ | 2841 |
| ELAGHDSPHKSGQNQQ | 2842 |
| TINGHDSPHKMGRNQQ | 2843 |
| TINGHDSPHKSGISIQ | 2844 |
| TSNGHDSPHKSVQNLQ | 2845 |
| QTQGHDSPAKSGQNQQ | 2846 |
| TINGHDSPHKMAHNQQ | 2847 |
| AINGSGSPHSKAQTQQ | 2848 |
| TINGHDSPHKHGQNQQ | 2849 |
| GADGHDSPHKSGQNQQ | 2850 |
| VGEGHDSPHKSGQNQQ | 2851 |
| ANEGHDSPAKSGQNQQ | 2852 |
| TEAKSGSPHSKAQNQQ | 2853 |
| TINGHDSPHKSVQSQQ | 2854 |
| TIPGSGSPHSKAQNLQ | 2855 |
| TINGHDSPHKSGQTVC | 2856 |
| ELRGHDSPHKSGQNQQ | 2857 |
| CQIGHDSPHKSGQNQQ | 2858 |
| GVMGHDSPRKSGQNQQ | 2859 |
| ACDGHDSPHKSGQNQQ | 2860 |
| TINGQDSPHKSGQYQQ | 2861 |
| TINGHDSPHKSGQQIM | 2862 |
| TINGHDSPHKSRQNEQ | 2863 |
| ASNGHDSPHKSGLNHQ | 2864 |
| TVNGHDSPHKSGQSQP | 2865 |
| NELGHDSPHKSGQNQQ | 2866 |
| AAEGHQSPHKSGQNQQ | 2867 |
| GQNGHDSPHKSGQNQQ | 2868 |
| NEFGHDSPHKSGQNQQ | 2869 |
| TSIGYDSPHKSGQNQQ | 2870 |
| TDNGHDSPHKSGQNLQ | 2871 |
| TITGHDSPHKSRQDQQ | 2872 |
| AEHGHDSPHKSGQNQQ | 2873 |
| TINGDDSPHKSGQKQL | 2874 |
| EILGHDSPHKSGQNQQ | 2875 |
| TIHGSGSPHSKAQNQA | 2876 |
| AINGDDSPHKSGHNQQ | 2877 |
| TSNGHNSPHKSGQNQE | 2878 |
| TINGHDSPHKSGQMIH | 2879 |
| NAIGHDSPHKSGQNQQ | 2880 |
| VINGHDSPHKSGRNHQ | 2881 |
| TITGHDSPRKSVQNRQ | 2882 |
| IINGHDSPHKSGQKMN | 2883 |
| TIHGHDSPHKSGQSQQ | 2884 |
| TEIGHDSPRKSGQNQQ | 2885 |
| TINGHDSPHKSGQYQH | 2886 |
| GSGSPHSKAQNRHT | 2887 |
| GSGSPHSKVQNQQT | 2888 |
| MSGSPHSKAQNQQT | 2889 |
| GSGSPHSKARDQQT | 2890 |
| GSGSPHSKAPNLQT | 2891 |
| TSGSPHSKAQNQQT | 2892 |
| GSGSPHSKAHVRQT | 2893 |
| GSGSPHSKAPNQHT | 2894 |
| ISGSPHSKAQNQQT | 2895 |
| GSGSPHSKTQSQQT | 2896 |
| ESGSPHSKAQNQQI | 2898 |
| GSGSPHSKAQPAKT | 2899 |
| SSGSPHSKAQNQQT | 2900 |
| GSGSPHSKSQTQQN | 2901 |
| ASGSPHSKAQNQQT | 2902 |
| GSGSPHSKAQNLAT | 2903 |
| GSGSPHSKSQNQLT | 2904 |
| NSGSPHSKAQNQQT | 2905 |
| GSGSPHSKAQGQQT | 2906 |
| VSGSPHSKAQNQQT | 2907 |
| GSGSPHSKALNRQS | 2908 |
| LSGSPRSKAQNQQT | 2909 |
| GSGSPHSKAHNQQT | 2910 |
| GSGSPHSKTQNQQT | 2911 |
| GSGSPHSKAQNPPT | 2912 |
| GSGSPHSKAQNLQT | 2913 |
| GSGSPHSKAQYQQT | 2914 |
| GSGSPHSKAQDQET | 2915 |
| KSGSPHSKAQNQQT | 2916 |
| GSGSPHSKAQNHQT | 2917 |
| GSGSPHSKAQDQQT | 2918 |
| GSGSPHSKGHWQQT | 2919 |
| GSGSPHSKAPNQQT | 2920 |
| GSGSPHSKAQNQLI | 2921 |
| GSGSPHSKRPEQQT | 2922 |
| GSGSPHSKAQRTMT | 2923 |
| GSGSPHSKAQNQQH | 2924 |
| HSGSPHSKAQNQQT | 2925 |
| GSGSPHSKALHQHT | 2926 |
| GSGSPHSKAQHRIT | 2927 |
| GSGSPHSKAQYIHT | 2928 |
| GSGSPHSKAQKFET | 2929 |
| ESGSPHSKAQNHQT | 2930 |
| GSGSPHSKAQFPST | 2931 |
| PSGSPHSKAQNQQT | 2932 |
| GSGSPHSKAHNQQR | 2933 |
| GSGSPHSKAQAIKT | 2934 |
| GSGSPHSKGQNRQT | 2935 |
| GSGSPHSKAQSQQT | 2936 |
| ASGSPHSKAQNQLT | 2937 |
| RSGSPHSKAQNQQT | 2938 |
| GSGSPHSKAQYQHT | 2939 |
| GSGSPHTKAQNPQS | 2940 |
| GSGSPHSKGQNPPT | 2941 |
| GSGSPHSKAQHQLT | 2942 |
| GSGSPHSKAQSPPT | 2943 |
| GSGSPHSKAQAKLI | 2944 |
| GSGSPHSKTKSQQT | 2945 |
| GSGSPHSKAQDRPT | 2946 |
| GSGSPHSKAQSQQL | 2947 |
| GSGSPHSKAQAFHT | 2948 |
| GSGSPHSKAQKQQD | 2949 |
| GSGSPHSKAQNAQT | 2950 |
| WSGSPHSKAQNQQT | 2951 |
| GSGSPHSKAHNQLT | 2952 |
| GSGSPHSKAQNQQY | 2953 |
| GSGSPHSKAQKLNT | 2954 |
| GSGSPHSKSQNVQT | 2955 |
| GSGSPHSKAQAQQT | 2956 |
| GSGSPHSKAQNLQA | 2957 |
| DSGSPHSKAQNQQT | 2958 |
| ASGSPHSKAPNQQT | 2959 |
| GSGSPHSKAQTPPT | 2960 |
| GSGSPHSKAQYQHA | 2961 |
| GSGSPHSKAQGQQA | 2962 |
| GSGSPHNKAQNHQT | 2963 |
| GSGSPHSKAQPPAT | 2964 |
| GSGSPHSKAQERPT | 2965 |
| GSGSPHSKAQDLQT | 2966 |
| GSGSPHSKAQAMHT | 2967 |
| GSGSPHSKALNQQT | 2968 |
| GSGSPHSKAQHPST | 2969 |
| GSGSPHSKAPGLQT | 2970 |
| GSGSPHSKAQGIRT | 2971 |
| GSGSPHSKAQAPAT | 2972 |
| GSGSPHSKSQSQQI | 2973 |
| GSGSPHSKAQIPPT | 2974 |
| GSGSPHSKAQTQLT | 2975 |
| GSGSPHSKAQAPST | 2976 |
| GSGSPHSKGQNQQT | 2977 |
| ASGSPHSKAHNQQT | 2978 |
| GSGSPHSKAQNTYA | 2979 |
| GSGSPHSKSQNQHI | 2980 |
| GSGSPHSKAQNHLT | 2981 |
| GSGSPHSKAQIGMT | 2982 |
| GSGSPHSKALNKQT | 2983 |
| GSGSPHSKAQLQQT | 2984 |
| GSGSPHSKAQRMST | 2985 |
| GSGSPHSKAQGILT | 2986 |
| GSGSPHSKAQDRQT | 2987 |
| GSGSPHSKARDWQT | 2988 |
| GSGSPHSKAQNTHD | 2989 |
| GSGSPHSKAPNLQI | 2990 |
| GSGSPHSKAQERST | 2991 |
| GSGSPHSKAQNYQT | 2992 |
| GSGSPHSKAQRTCT | 2993 |
| GSGSPHSKAQIGHT | 2994 |
| GSGSPHSKAQGAIT | 2995 |
| GSGSPHSKAQVPPT | 2996 |
| GSGSPHSKAQVQQI | 2997 |
| GSGSPHSKALMRQT | 2998 |
| GSGSPHSKAQYSVT | 2999 |
| GSGSPHSKVPNLQT | 3000 |
| GSGSPHSKAQSQQI | 3001 |
| GSGSPHSKAQAITT | 3002 |
| GSGSPHSKAQKTLT | 3003 |
| GSGSPHSKAQNQWT | 3004 |
| GSGSPHSKAQLHHT | 3005 |
| GSGSPHSKAQNIII | 3006 |
| GSGSPHSKAQGHHT | 3007 |
| GSGSPHSKAQSKVT | 3008 |
| GSGSPHSKAQLPST | 3009 |
| GSGSPHSKAIGKQT | 3010 |
| GSGSPHSKAQAIHT | 3011 |
| GSGSPHSKAQHGLT | 3012 |
| GSGSPHSKAQFMCT | 3013 |
| VSGSPHSKAQGQQT | 3014 |
| GSGSPHSKAQHLQT | 3015 |
| GSGSPHSKTQNHQN | 3016 |
| GSGSPHSKAQPART | 3017 |
| GSGSPHSKAQSLQT | 3018 |
| GSGSPHSKSQSQLT | 3019 |
| GSGSPHSKAQDRQS | 3020 |
| GSGSPHSKAQMPST | 3021 |
| GSGSPHSKAQGSLT | 3022 |
| GSGSPHSKSQNQQT | 3023 |
| GSGSPHSKAQVPAT | 3024 |
| GSGSPHSKAQDKQT | 3025 |
| GSGSPHSKAHYQQT | 3026 |
| GSGSPHSKAQVPST | 3027 |
| GSGSPHSKARGEQT | 3028 |
| GSGSPHSKAPGQQT | 3029 |
| GSGSPHSKAQSLQI | 3030 |
| GSGSPHSKRLEQQT | 3031 |
| GSGSPHSKAQNQST | 3032 |
| GSGSPHSKAQKVIT | 3033 |
| GSGSPHSKAQNNDQ | 3034 |
| GSGSPHSKAQSVHT | 3035 |
| GSGSPHSKAQPLGT | 3036 |
| GSGSPHSKAHNQET | 3037 |
| GSGSPHSKAQNLQI | 3038 |
| GSGSPHSKAQIQQT | 3039 |
| GSGSPHSKAQVRNT | 3040 |
| GSGSPHSKAPSNQT | 3041 |
| GSGSPHSKAQVGHT | 3042 |
| GSGSPHSKAQRDIT | 3043 |
| GSGSPHSKAQPNTM | 3044 |
| GSGSPHSKARGLQT | 3045 |
| GSGSPHSKLQKQQT | 3046 |
| GSGSPHSKAPSLQT | 3047 |
| GSGSPHSKAQRDQT | 3048 |
| GSGSPHSKNRDQQT | 3049 |
| GSGSPHSKAQAKGT | 3050 |
| GSGSPHSKAQSAHT | 3051 |
| GSGSPHSKSQNHQT | 3052 |
| RSGSPHSKAQDQQT | 3053 |
| GSGSPHSKAQSTMT | 3054 |
| GSGSPHSKAQREMT | 3055 |
| GSGSPHSKAQYRAT | 3056 |
| GSGSPHSKNQWQQT | 3057 |
| GSGSPHSKAQRMNT | 3058 |
| GSGSPHAKAQNHQT | 3059 |
| GSGSPHSKAGDSQT | 3060 |
| GSGSPHSKLKSQQT | 3061 |
| GSGSPHSKAQKSTI | 3062 |
| GSGSPHSKAPSMQT | 3063 |
| GSGSPHSKASPRQT | 3064 |
| GSGSPHSKRMEQQT | 3065 |
| GSGSPHSKAQYQNT | 3066 |
| RSGSPHSKAQNQQI | 3067 |
| GSGSPHTKAQNPPT | 3068 |
| GSGSPHTKAQNQQT | 3069 |
| ASGSPHSKAQHQQT | 3070 |
| ASGSPHSKAINQQT | 3071 |
| GSGSPHSKAPNQQH | 3072 |
| ASGSPHSKAQNHQT | 3073 |
| GSGSPHSKAENRQT | 3074 |
| GSGSPHSKVQDQQT | 3075 |
| GSGSPHSKAQTQQA | 3076 |
| GSGSPHSKAQNQAT | 3077 |
| GHDSPHKSGQIRQT | 3078 |
| GHDSPHKIGQNQHA | 3079 |
| GYDSPHKSGQKQQT | 3080 |
| GHDSPHKSGQNQQT | 3081 |
| GHDSPHKSGQSVQT | 3082 |
| GHESPHKSGRSQQT | 3083 |
| GHDSPHKSGQNKLE | 3084 |
| GHDSPHKTGQNQQK | 3085 |
| GRGSPHKRGQNQQT | 3086 |
| GHDNPHKSGQNQQT | 3087 |
| GQDSPHKSGQHQQA | 3088 |
| GHDSPHKSGQIQHT | 3089 |
| GHDSPHKSGPRQQT | 3090 |
| GHDSPHKSGHTQQT | 3091 |
| GHDSPHKSGQRQHT | 3092 |
| GHDSPHKSAQSQQT | 3093 |
| GHESPHKSGQNQQS | 3094 |
| GHDSPHKSLQNQQT | 3095 |
| GHDSPHKSGRNQET | 3096 |
| GHDSPHKSGQTQLT | 3097 |
| GHDSPHKSEKNQQT | 3098 |
| GRDSPHKSGQDQQT | 3099 |
| GHDSPHKTGHNQQT | 3100 |
| AHDSPHKSGQNQLT | 3101 |
| GYDSPHKSGQTQQT | 3102 |
| GHESPHKSGQTQQI | 3103 |
| GHDSPHKSGQSKQA | 3104 |
| GHESPHKSGQNQQT | 3105 |
| GHDSPHKTGQNQPP | 3106 |
| GHDSPHKSGQSPQT | 3107 |
| GNDSPHKSVQNQQT | 3108 |
| GHDSPHKSAQNYQT | 3109 |
| AHDSPHKIGQNHQT | 3110 |
| GHESPHKSAQNRQT | 3111 |
| GHDSPHKSGQNQQG | 3112 |
| GQDSPHKIGQNQQT | 3113 |
| GHDSPHKSGQNHLT | 3114 |
| GHDSPHKSGQYQHT | 3115 |
| GNDSPHKSVQNHQT | 3116 |
| GHDSPHKSGQNQWT | 3117 |
| GHDSPHKSVQNQHT | 3118 |
| GHDSPHKSGQNQHA | 3119 |
| GHDSPHKSGQTRQT | 3120 |
| GHDSPHKSGQNLHT | 3121 |
| GHDSPHKSGLNQQT | 3122 |
| GHDSPHKSAQNQET | 3123 |
| GHDSPHKSGQHLQT | 3124 |
| GHDSPHKSRLNQPT | 3125 |
| GHDSPHKSGQKQQT | 3126 |
| GQDSPHKSGQNQDT | 3127 |
| GHDSPHKSGRNQQT | 3128 |
| GHDSPHKSGQNLLT | 3129 |
| GHDSPHKSGQLVIT | 3130 |
| GHDSPHKSRQSQQT | 3131 |
| GHDSPHKSGRTQET | 3132 |
| GHDSPHKSVQTHQT | 3133 |
| GHDSPHKSGQNQPA | 3134 |
| KHDSPHKSGQNQQT | 3135 |
| GHDSPHKSGQTQQT | 3136 |
| GPDSPHKIGQNQQS | 3137 |
| GHDSPHKSVQNQQT | 3138 |
| GHDSPHKSRQDQHT | 3139 |
| GPDSPHKSGQKQQT | 3140 |
| GHDSPHKSRQSQHT | 3141 |
| GHDSPHKSVQNQLT | 3142 |
| AHDSPHKSGQNQQT | 3143 |
| GQDSPHKSGQNQQS | 3144 |
| GHDSPHKSGRNQQI | 3145 |
| GYDSPHKSGQYQHT | 3146 |
| GHDSPHKSRQNQQT | 3147 |
| GHDSPHKSWVRQQT | 3148 |
| GHESPHKSGQNQHS | 3149 |
| GHDSPHKIGHNQQT | 3150 |
| WHDSPHKSGQNQQT | 3151 |
| RHDSPHKSGQNQQT | 3152 |
| GHDSPHKSNAWQQT | 3153 |
| GHDSPHKSGQSVPT | 3154 |
| GHESPHKSGQNIQP | 3155 |
| GHDSPHKSVQNHLN | 3156 |
| GHDSPHKIGLDQQT | 3157 |
| GHDSPHKRGPDQQS | 3158 |
| GHDSPHKSGESQQT | 3159 |
| GHDSPHKHGQNHQT | 3160 |
| LHDSPHKSGQNQQT | 3161 |
| GHDSPHKSVQNKQT | 3162 |
| GQVSPHKSGQNQQT | 3163 |
| GHDSPHKSGQRQLT | 3164 |
| GHDSPHKIGQNQLT | 3165 |
| GHDSPHKSGQIIVT | 3166 |
| IHDSPHKSGQNQQT | 3167 |
| GYDSPHKSGQKQST | 3168 |
| MHDSPHKSGQNQQT | 3169 |
| GDDSPHKSVQNQQT | 3170 |
| GHDSPHKSVQSHQT | 3171 |
| GHDSPHKSGQFVVT | 3172 |
| GHDSPHKSRQNLQT | 3173 |
| GHNSPHKSGQNQET | 3174 |
| GHDSPHKSAQNQQI | 3175 |
| NHDSPHKSGQNQQT | 3176 |
| GHDSPHKSGQSQPT | 3177 |
| GHESPRKSGQNEQT | 3178 |
| GHDSPHKSAQNQQT | 3179 |
| GHDSPHKSGQNQLT | 3180 |
| GHDSPHKSAQNLLT | 3181 |
| THDSPRKSGQNQQT | 3182 |
| GNGSPHKRGQNQQT | 3183 |
| GHDSPHKSGQSLQT | 3184 |
| GHDSPHKSAQNHQT | 3185 |
| GHDSPHKSGRNRQT | 3186 |
| EHDSPRKSGQNQQI | 3187 |
| GHDSPHKYGQNEQT | 3188 |
| RHDSPHKSGHNQQT | 3189 |
| GHDSPHKSQQNQQT | 3190 |
| GHDSPHKSGQNQQI | 3191 |
| QHDSPHKSGQNQQT | 3192 |
| RHDSPHKIVQNQQT | 3193 |
| YHDSPHKSGQNQQT | 3194 |
| GHDSPHKSVQSKQT | 3195 |
| GNDSPHKIGHNQQT | 3196 |
| HHDSPHKSGQNQQT | 3197 |
| GHDSPHKSGQTQQI | 3198 |
| GQDSPHKSGQNPLT | 3199 |
| GHDSPHKSGRDQKT | 3200 |
| GHDSPHKSVHNQQN | 3201 |
| GHDSPHKSGQWKRT | 3202 |
| GHDSPHKSGQSQQI | 3203 |
| GHDSPHKSRQAQQT | 3204 |
| GHDSPHKSVQNHQI | 3205 |
| GHDSPHKSKQNQQA | 3206 |
| GHDSPHKSAQNQLN | 3207 |
| GHDSPHKSGQTQPT | 3208 |
| FHDSPHKSGQNQQT | 3209 |
| GHDSPHKLWINQQT | 3210 |
| GPDSPHKSGQNQQT | 3211 |
| GHDSPHKSVQKQLT | 3212 |
| GHDSPHKSGRNQLA | 3213 |
| VHDSPHKSGQNQQS | 3214 |
| GHDSPHKMGRNQQS | 3215 |
| GHDSPHKSGISIQT | 3216 |
| VHDSPHKSGQNQQT | 3217 |
| GHDSPHKSVQNLQT | 3218 |
| GHDSPHKMAHNQQT | 3219 |
| GHDSPHKHGQNQQN | 3220 |
| GHDSPHKSVQSQQS | 3221 |
| GHDSPHKSGQTVCT | 3222 |
| GQDSPHKSGQYQQI | 3223 |
| GHDSPHKSGQQIMT | 3224 |
| GHDSPHKSRQNEQS | 3225 |
| GHDSPHKSGLNHQT | 3226 |
| GYDSPHKSGQNQQT | 3227 |
| GHDSPHKSGQNLQT | 3228 |
| GHDSPHKSRQDQQT | 3229 |
| GDDSPHKSGQKQLT | 3230 |
| GDDSPHKSGHNQQT | 3231 |
| GHDSPHKSGQMIHT | 3232 |
| GHDSPHKSGRNHQS | 3233 |
| GHDSPHKSVQNRQT | 3234 |
| GHDSPHKSGQKMNT | 3235 |
| GHDSPHKSGQSQQN | 3236 |
| GHDSPHKSGQYQHA | 3237 |
| KTISKRGSPHSKAQNQQT | 4098 |
| KGLGGSGSPHSKAQNQQT | 4099 |
| KTINGHDSPHSKAQNLQT | 4100 |
| KTINGHDSPHSKAQNQQI | 4101 |
| KTINGSGSPHFTRQNQQT | 4102 |
| KTINGSGSPHSLPWNQQT | 4103 |
| KTINGHDSPHSKAQNHQT | 4104 |
| KTMNGHDSPHSKAQNQQT | 4105 |
| KPYKGSGSPHSKAQNQQT | 4106 |
| KRLWGSGSPHSKAQNQQT | 4107 |
| KRMRGSGSPHSKAQNQQT | 4108 |
| KRTYGSGSPHSKAQNQQT | 4109 |
| KTINCLRSPHSKAQNQQT | 4110 |
| KTINFSRSPHSKAQNQQT | 4111 |
| KTINGLRSPRFKAQNQQT | 4112 |
| KTINGNRSPHNKAQNQQT | 4113 |
| KTINGPRSPHYKAQNQQT | 4114 |
| KTINGQASPHWKAQNQQT | 4115 |
| KTINGRCSPHSKAQNQQT | 4116 |
| KTINGRHSPHSKAQNQQT | 4117 |
| KTINGRKSPHRKAQNQQT | 4118 |
| KTINGRLSPHWKAQNQQT | 4119 |
| KTINGRLSPRYKAQNQQT | 4120 |
| KTINGRPSPHMKAQNQQT | 4121 |
| KTINGRSSPHWKAQNQQT | 4122 |
| KTINGRWSPHSKAQNQQT | 4123 |
| KTINGSGSPRAPCQNQQT | 4124 |
| KTINGSGSPRAWAQNQQT | 4125 |
| KTINGSGSPHCMRQNQQT | 4126 |
| KTINGSGSPRFCSQNQQT | 4127 |
| KTINGSGSPRFLFQNQQT | 4128 |
| KTINGSGSPHFWAQNQQT | 4129 |
| KTINGSGSPHLCAQNQQT | 4130 |
| KTINGSGSPHLRYQNQQT | 4131 |
| KTINGSGSPHLYYQNQQT | 4132 |
| KTINGSGSPHPLCQNQQT | 4133 |
| KTINGSGSPHRIRQNQQT | 4134 |
| KTINGSGSPHRLFQNQQT | 4135 |
| KTINGSGSPHSCGQNQQT | 4136 |
| KTINGSGSPHSCLRNQQT | 4137 |
| KTINGSGSPHSCLSNQQT | 4138 |
| KTINGSGSPHSCRLNQQT | 4139 |
| KTINGSGSPHSCSLNQQT | 4140 |
| KTINGSGSPHSKACTLQT | 4141 |
| KTINGSGSPHSKAFRAQT | 4142 |
| KTINGSGSPHSKAIRKQT | 4143 |
| KTINGSGSPHSKAQASRT | 4144 |
| KTINGSGSPHSKAQFELT | 4145 |
| KTINGSGSPHSKAQIVIT | 4146 |
| KTINGSGSPHSKAQARTL | 4147 |
| KTINGSGSPHSKAQLQRT | 4148 |
| KTINGSGSPHSKAQNARR | 4149 |
| KTINGSGSPHSKAQNCPR | 4150 |
| KTINGSGSPHSKAQNMRR | 4151 |
| KTINGSGSPHSKAQNRRV | 4152 |
| KTINGSGSPHSKAQPSRT | 4153 |
| KTINGSGSPHSKAQQVKT | 4154 |
| KTINGSGSPHSKAQQVRT | 4155 |
| KTINGSGSPHSKAQRLKT | 4156 |
| KTINGSGSPHSKAQRRAT | 4157 |
| KTINGSGSPHSKAQRRGT | 4158 |
| KTINGSGSPHSKAQRRRT | 4159 |
| KTINGSGSPHSKAQRTRT | 4160 |
| KTINGSGSPHSKAQRVHT | 4161 |
| KTINGSGSPHSKAQTYRT | 4162 |
| KTINGSGSPHSKAQVRKT | 4163 |
| KTINGSGSPHSKARGRQT | 4164 |
| KTINGSGSPHSKARLCQT | 4165 |
| KTINGSGSPHSKARLKQT | 4166 |
| KTINGSGSPHSKARNSQT | 4167 |
| KTINGSGSPHSKARWVQT | 4168 |
| KTINGSGSPHSKAVRWQT | 4169 |
| KTINGSGSPHSKAYTRQT | 4170 |
| KTINGSGSPHSKCQSQQT | 4171 |
| KTINGSGSPHSKFLRQQT | 4172 |
| KTINGSGSPHSKFRFQQT | 4173 |
| KTINGSGSPHSKFRLQQT | 4174 |
| KTINGSGSPHSKFRRQQT | 4175 |
| KTINGSGSPHSKGMKQQT | 4176 |
| KTINGSGSPHSKKLRQQT | 4177 |
| KTINGSGSPHSKKRPQQT | 4178 |
| KTINGSGSPHSKKSRQQT | 4179 |
| KTINGSGSPHSKLYRQQT | 4180 |
| KTINGSGSPHSKLYWQQT | 4181 |
| KTINGSGSPHSKPRMQQT | 4182 |
| KTINGSGSPRSKRFPQQT | 4183 |
| KTINGSGSPHSKRFRQQT | 4184 |
| KTINGSGSPHSKRPYQQT | 4185 |
| KTINGSGSPHSKRRMQQT | 4186 |
| KTINGSGSPHSKRSKQQT | 4187 |
| KTINGSGSPHSKRSRQQT | 4188 |
| KTINGSGSPHSKRTMQQT | 4189 |
| KTINGSGSPHSKRTRQQT | 4190 |
| KTINGSGSPHSKRVRQQT | 4191 |
| KTINGSGSPHSKRYIQQT | 4192 |
| KTINGSGSPHSKRYNQQT | 4193 |
| KTINGSGSPHSKRYPQQT | 4194 |
| KTINGSGSPHSKRYSQQT | 4195 |
| KTINGSGSPHSKTCIQQT | 4196 |
| KTINGSGSPHSKWLTQQT | 4197 |
| KTINGSGSPHSKWVVQQT | 4198 |
| KTINGSGSPHSKYRLQQT | 4199 |
| KTINGSGSPHSKYSKQQT | 4200 |
| KTINGSGSPHSKYSRQQT | 4201 |
| KTINGSGSPHSLKRNQQT | 4202 |
| KTINGSGSPHSLWENQQT | 4203 |
| KTINGSGSPHSLWPNQQT | 4204 |
| KTINGSGSPHSLWTNQQT | 4205 |
| KTINGSGSPHSMRRNQQT | 4206 |
| KTINGSGSPHSPCLNQQT | 4207 |
| KTINGSGSPHSQWQNQQT | 4208 |
| KTINGSGSPHSRCANQQT | 4209 |
| KTINGSGSPHSRIRNQQT | 4210 |
| KTINGSGSPHSRKSNQQT | 4211 |
| KTINGSGSPHSRLWNQQT | 4212 |
| KTINGSGSPHSRRENQQT | 4213 |
| KTINGSGSPHSRRPNQQT | 4214 |
| KTINGSGSPHSRSQNQQT | 4215 |
| KTINGSGSPHSRSKNQQT | 4216 |
| KTINGSGSPHSRTKNQQT | 4217 |
| KTINGSGSPHSRWLNQQT | 4218 |
| KTINGSGSPHSSVCNQQT | 4219 |
| KTINGSGSPHSSWRNQQT | 4220 |
| KTINGSGSPHSVCQNQQT | 4221 |
| KTINGSGSPHSVLQNQQT | 4222 |
| KTINGSGSPHSVRRNQQT | 4223 |
| KTINGSGSPHSVSCNQQT | 4224 |
| KTINGSGSPHSWALNQQT | 4225 |
| KTINGSGSPHSWITNQQT | 4226 |
| KTINGSGSPHSWPMNQQT | 4227 |
| KTINGSGSPHSWRSNQQT | 4228 |
| KTINGSGSPHSYFLNQQT | 4229 |
| KTINGSGSPHSYTYNQQT | 4230 |
| KTINGSGSPHSYWQNQQT | 4231 |
| KTINGSGSPHTLCQNQQT | 4232 |
| KTINGSGSPHWLRQNQQT | 4233 |
| KTINGSGSPHWPSQNQQT | 4234 |
| KTINGSGSPHYLRQNQQT | 4235 |
| KTINGSGSPHYTRQNQQT | 4236 |
| KTINGSLSPHLWAQNQQT | 4237 |
| KTINGSPSPHCQAQNQQT | 4238 |
| KTINGSRSPHLCAQNQQT | 4239 |
| KTINGSRSPHWRAQNQQT | 4240 |
| KTINGSVSPHWLAQNQQT | 4241 |
| KTINGTESPHRKAQNQQT | 4242 |
| KTINGWTSPHRKAQNQQT | 4243 |
| KTINRGISPHSKAQNQQT | 4244 |
| KTINTVRSPHSKAQNQQT | 4245 |
| KTKLRSGSPHSKAQNQQT | 4246 |
| KTRLRSGSPHSKAQNQQT | 4247 |
| KWLLGSGSPHSKAQNQQT | 4248 |
| KWSQGSGSPHSKAQNQQT | 4249 |
| KWYLGSGSPHSKANQQTQ | 4250 |
| KYHSGSGSPHSKAQNQQT | 4251 |
| KYLPGSGSPHSKAQNQQT | 4252 |
| KAINGGGSPHSKTQNQQT | 4253 |
| KAINGRDSPHERSPNQQT | 4254 |
| KAINGHDSPHKSFSPQQT | 4255 |
| KAINGHDSPHKSGENQQP | 4256 |
| KAINGHDSPHKSGQLART | 4257 |
| KAINGHDSPHKSGQNAFL | 4258 |
| KAINGHDSPHKSGQNAYT | 4259 |
| KAINGHDSPHKSGQNFAS | 4260 |
| KAINGHDSPHKSGQNLAS | 4261 |
| KAINGHDSPHKSGQNLGS | 4262 |
| KAINGHDSPHKSGQNLKF | 4263 |
| KAINGHDSPHKSGQNLLK | 4264 |
| KAINGHDSPHKSGQNLSR | 4265 |
| KAINGHDSPHKSGQNLSS | 4266 |
| KAINGHDSPHKSGQNSLG | 4267 |
| KAINGHDSPHKSGQNTLQ | 4268 |
| KAINGHDSPHKSGQNTSL | 4269 |
| KAINGHDSPHKSGQRLGT | 4270 |
| KAINGHDSPHKSGQRNYT | 4271 |
| KAINGHDSPHKSGQRPST | 4272 |
| KAINGHDSPHKSGQRPVT | 4273 |
| KAINGHDSPHKSGQVPST | 4274 |
| KAINGHDSPHKSLSNQQT | 4275 |
| KAINGHDSPHKSVLSQQT | 4276 |
| KAINGHDSPHKTLQNQQT | 4277 |
| KAINGHNSPHSKAQNQQT | 4278 |
| KAINGLDSPHSKAQNQQT | 4279 |
| KAINGSGSPHKSGQNQQT | 4280 |
| KAINGSGSPHSKAQGQQT | 4281 |
| KAINGSGSPHSKAQLSGT | 4282 |
| KAINGSGSPHSKAQNGSL | 4283 |
| KAINGSGSPHSKAQNSLL | 4284 |
| KAINGSGSPHSKAVGLQT | 4285 |
| KAINGSGSPHSKSLLQQT | 4286 |
| KAINGSGSPHSKSLPQQT | 4287 |
| KAINGSGSPHSKSTFQQT | 4288 |
| KAITGHDSPHSKAQNQQT | 4289 |
| KDVMGSGSPHSKAQNQQT | 4290 |
| KEIVGSGSPHSKAQNQQT | 4291 |
| KEIYGSGSPHSKAQNQQT | 4292 |
| KELSGSGSPHSKAQNQQT | 4293 |
| KETIGSGSPHSKAQNQQT | 4294 |
| KEVLGSGSPHSKAQNQQT | 4295 |
| KFALGHDSPHKSGQKQQT | 4296 |
| KIINGHDSPHKSGQNLVL | 4297 |
| KIINGHDSPHKSGQRNYT | 4298 |
| KIINGHDSPHSKAQNQQT | 4299 |
| KLNPGHDSPHKSGQTQQT | 4300 |
| KLNRGHDSPHKSGQNQQS | 4301 |
| KLSSGHDSPHKSGQNQQN | 4302 |
| KNINGHDSPHSKAQNQQT | 4303 |
| KNNDGSGSPHSKAQNQQT | 4304 |
| KNVMGSGSPHSKAQNQQT | 4305 |
| KPINGHDSPHKSGQNKLS | 4306 |
| KPINGHDSPHKSGQNLSS | 4307 |
| KPINGHDSPHSKAQNQQT | 4308 |
| KRINGRDSPHSKAQNQQT | 4309 |
| KSCSGHDSPAKSGQNQQS | 4310 |
| KSINGHDSPHKSCQNLAS | 4311 |
| KSINGHDSPHKSGQNLFL | 4312 |
| KSINGHDSPHKSCQNLLM | 4313 |
| KSINGHDSPHKSGQNLLQ | 4314 |
| KSINGHDSPHKSGQNSLG | 4315 |
| KSINGHDSPHKSGQNTLQ | 4316 |
| KSINGHDSPHKSSSNQQT | 4317 |
| KSINGHDSPHKYKLNQQT | 4318 |
| KSINGSGSPHKSGQKQQT | 4319 |
| KSINGSGSPHKSGQNQQT | 4320 |
| KSINGSGSPHSKAQGLST | 4321 |
| KSINGSGSPHSKAQLLGT | 4322 |
| KSINGSGSPHSKTSWQQT | 4323 |
| KSMNGHDSPHSKAQNQQT | 4324 |
| KSTLGSGSPHSKAQNQHT | 4325 |
| KSTLGSGSPHSKAQNQQN | 4326 |
| KSTVGSGSPHSKAQTQQT | 4327 |
| KTCKESGSPHSKAQNQQT | 4328 |
| KICKGSGSPHSKAQNQQT | 4329 |
| KTCKSSGSPHSKAQNQQT | 4330 |
| KTDMGSGSPHSKAQNQQT | 4331 |
| KTDNGIGSPHSKAQNQQT | 4332 |
| KTEGGSGSPHSKAQNQQT | 4333 |
| KTEHHSGSPHSKAQNQQT | 4334 |
| KTEKDSGSPHSKAQNQQT | 4335 |
| KTELGHDSPHKRGQNQQT | 4336 |
| KTESVSGSPHSKAQNQQT | 4337 |
| KTETNSGSPHSKAQNQQT | 4338 |
| KTETYSGSPHSKAQNQQT | 4339 |
| KTEWLSGSPHSKAQNQQT | 4340 |
| KTFNGSGSPHKSGQNQQT | 4341 |
| KTGLRHDSPHKSGQKQQT | 4342 |
| KTGLRHDSPHKSGQNQQS | 4343 |
| KTGVTADSPHKSGQKQQT | 4344 |
| KTIDGHESPHSKAQNQQT | 4345 |
| KTIEGHDSPHKSGQTQQT | 4346 |
| KTIHGHDSPHSKAQNQQT | 4347 |
| KTIHGHESPHSKAQNQQT | 4348 |
| KTHIGHDSPRKSGQNRSS | 4349 |
| KTHIGHDSPRKSGQRLGT | 4350 |
| KTIIGSGSPHKSGQNQQT | 4351 |
| KTIKGHDSPHKSCQNMLF | 4352 |
| KTILGSGSPHSKAQNLQT | 4353 |
| KTINGCSSPHWKAQNQQT | 4354 |
| KTINGGGSTHSKAQNQQT | 4355 |
| KTINGHDSPHKAGQSQQT | 4356 |
| KTINGHDSPHKRGQNVPS | 4357 |
| KTINGHDSPHKRGRSYQT | 4358 |
| KTINGHDSPHKTGQNPPT | 4359 |
| KTINGHDSPHSKAENQQT | 4360 |
| KTINGHDSPHSKALSLQT | 4361 |
| KTINGHDSPHSKAQGQQT | 4362 |
| KTINGHDSPHSKAQHQQT | 4363 |
| KTINGHDSPHSKAQIQQT | 4364 |
| KTINGHDSPHSKAQKQQT | 4365 |
| KTINGHDSPHSKAQNLSS | 4366 |
| KTINGHDSPHSKAQNPQT | 4367 |
| KTINGHDSPHSKAQNQET | 4368 |
| KTINGHDSPHSKAQNQHT | 4369 |
| KTINGHDSPHSKAQNQLT | 4370 |
| KTINGHDSPHSKAQNQPT | 4371 |
| KTINGHDSPHSKAQNQQA | 4372 |
| KTINGHDSPHSKAQNTGS | 4373 |
| KTINGHDSPHSKAQSQQT | 4374 |
| KTINGHDSPHSKAQTQQT | 4375 |
| KTINGHDSPHSKAQYQQT | 4376 |
| KTINGHDSPHSKARNQQT | 4377 |
| KTINGHDSPHSKLPGQQT | 4378 |
| KTINGHDSPHSKSPNQQT | 4379 |
| KTINGHESPHKSGQNAFL | 4380 |
| KTINGIGSPRSKAPNEQT | 4381 |
| KTINGQDSPHKSCQNLHM | 4382 |
| KTINGRGSPHSKAQIGMT | 4383 |
| KTINGRGSPHSKAQNQVL | 4384 |
| KTINGRGSPHSKAQSPTT | 4385 |
| KTINGRGSPHSKATSFQT | 4386 |
| KTINGSGSPHFVVQNQQT | 4387 |
| KTINGSGSPHKRGQKQQT | 4388 |
| KTINGSGSPHKRGQNQET | 4389 |
| KTINGSGSPHKRGQNQLT | 4390 |
| KTINGSGSPHKRGRNQQT | 4391 |
| KTINGSGSPHKSGGNQQT | 4392 |
| KTINGSGSPHKSGHNQET | 4393 |
| KTINGSGSPHKSGHNQLT | 4394 |
| KTINGSGSPHKSGHNQQN | 4395 |
| KTINGSGSPHKSGLNQLT | 4396 |
| KTINGSGSPHKSGPNQQT | 4397 |
| KTINGSGSPHKSGQGQQT | 4398 |
| KTINGSGSPHKSGQHLQT | 4399 |
| KTINGSGSPHKSGQHQQT | 4400 |
| KTINGSGSPHKSGQKHQT | 4401 |
| KTINGSGSPHKSGQKQQS | 4402 |
| KTINGSGSPHKSGQNEQT | 4403 |
| KTINGSGSPHKSGQNHQT | 4404 |
| KTINGSGSPHKSGQNKQT | 4405 |
| KTINGSGSPHKSGQNKTS | 4406 |
| KTINGSGSPHKSGQNQEA | 4407 |
| KTINGSGSPHKGQNQETS | 4408 |
| KTINGSGSPHKSGQNQKT | 4409 |
| KTINGSGSPHKSGQNQQI | 4410 |
| KTINGSGSPHKSGQNQQR | 4411 |
| KTINGSGSPHKSGQNQRT | 4412 |
| KTINGSGSPHKSGQNQYT | 4413 |
| KTINGSGSPHKSGQRQQT | 4414 |
| KTINGSGSPHKSGQSQQT | 4415 |
| KTINGSGSPHKSGQYQRT | 4416 |
| KTINGSGSPHKSGRNQQA | 4417 |
| KTINGSGSPHKSRHNQQT | 4418 |
| KTINGSGSPHKSRQYQQT | 4419 |
| KTINGSGSPHRKAQAPGT | 4420 |
| KTINGSGSPHSKAAMKQT | 4421 |
| KTINGSGSPHSKAGRQQT | 4422 |
| KTINGSGSPHSKAGRTQT | 4423 |
| KTINGSGSPHSKAKSNQT | 4424 |
| KTINGSGSPHSKALKTQT | 4425 |
| KTINGSGSPHSKAPRTQT | 4426 |
| KTINGSGSPHSKAQAART | 4427 |
| KTINGSGSPHSKAQAILT | 4428 |
| KTINGSGSPHSKAQCRGT | 4429 |
| KTINGSGSPHSKAQGLRT | 4430 |
| KTINGSGSPHSKAQKGVL | 4431 |
| KTINGSGSPHSKAQKSNT | 4432 |
| KTINGSGSPHSKAQNNKF | 4433 |
| KTINGSGSPHSKAQNRRT | 4434 |
| KTINGSGSPHSKAQPKQT | 4435 |
| KTINGSGSPHSKAQRAPT | 4436 |
| KTINGSGSPHSKAQREHT | 4437 |
| KTINGSGSPHSKAQRFGT | 4438 |
| KTINGSGSPHSKAQRPCT | 4439 |
| KTINGSGSPHSKAQRQAT | 4440 |
| KTINGSGSPHSKAQRQPT | 4441 |
| KTINGSGSPHSKAQTKLT | 4442 |
| KTINGSGSPHSKAQTTHT | 4443 |
| KTINGSGSPHSKAQVQRT | 4444 |
| KTINGSGSPHSKAQVVRT | 4445 |
| KTINGSGSPHSKAQWPNT | 4446 |
| KTINGSGSPHSKAQYPST | 4447 |
| KTINGSGSPHSKARALQT | 4448 |
| KTINGSGSPHSKARDQHT | 4449 |
| KTINGSGSPHSKARFQQT | 4450 |
| KTINGSGSPHSKARRTQT | 4451 |
| KTINGSGSPHSKARSLQT | 4452 |
| KTINGSGSPHSKARVIQT | 4453 |
| KTINGSGSPHSKAWYLQT | 4454 |
| KTINGSGSPHSKGGGQQT | 4455 |
| KTINGSGSPHSKGSRQQT | 4456 |
| KTINGSGSPHSKLQRQQT | 4457 |
| KTINGSGSPHSKMLRQQT | 4458 |
| KTINGSGSPHSKSSIKQT | 4459 |
| KTINGSGSPHSKVRFQQT | 4460 |
| KTINGSGSPHSVVWNQQT | 4461 |
| KTINGSTSPHKLAQNQQP | 4462 |
| KTINRHDSPHKSGQRPST | 4463 |
| KTINRIMSPHSKAQNQQT | 4464 |
| KTINTARSPHSKAQNQQT | 4465 |
| KTISGHDSPHSKAQNQQT | 4466 |
| KTISGSGSPHKSGQNQQT | 4467 |
| KTITGHDSPHKSGQRLGT | 4468 |
| KTITGSGSPHKSGQNQQT | 4469 |
| KTIYGHDSPHKSGQRLGT | 4470 |
| KTLNGHDSPHKSGQNLFL | 4471 |
| KTLNGHDSPHKSGQNLSS | 4472 |
| KTLSFHDSPRKSGQNQQS | 4473 |
| KTSNGSGSPHSKAQNTMT | 4474 |
| KTTNGHDSPHSKAQNQQT | 4475 |
| KTVNGGGSPHSKAQNQQT | 4476 |
| KTVNGHDSPHKSGQNVSL | 4477 |
| KTVNGHDSPHKSGQRPST | 4478 |
| KTVNGHDSPHKSGQTQQA | 4479 |
| KTVNGHESPHSKAQNQQT | 4480 |
| KTVNGSGSPHSKAQGLST | 4481 |
| KTVNGSGSPHSKAQNVTS | 4482 |
| KTVPASGSPHSKAQNQQT | 4483 |
| NTINGSGSPHSKAHNQQT | 4484 |
| TTINGGGSPHSKAQNQQT | 4485 |

TABLE 2A
Exemplary Sequences

| SEQ ID | Amino Acid | SEQ ID | |
|---|---|---|---|
| NO: | Sequence | NO: | Nucleotide Sequence |
| 941 | SPHSKA | 942 | AGCCCACACAGCAAAGCA |
| 943 | HDSPHKSG | 944 | CACGACAGCCCACACAAAA |
| | | | GCGGA |
| 2 | HDSPHK | 3 | CACGACAGCCCACACAAA |

TABLE 2B
Exemplary Sequences

| | SEQ |
|---|---|
| Amino Acid | ID |
| Sequence | NO: |
| SPHSKA | 945 |
| SPHKSG | 946 |
| SPHARM | 947 |
| SPHVKS | 948 |
| SPHASR | 949 |
| SPHVKI | 950 |
| SPHKSR | 951 |
| SPHSLR | 952 |
| SPHSKW | 953 |
| SPHKKN | 954 |
| SPHVRM | 955 |
| SPHRKA | 956 |
| SPHKFG | 957 |
| SPHKIG | 958 |
| SPHKLG | 959 |
| SPHSKL | 960 |
| SPHSRG | 961 |
| SPHSKS | 962 |
| SPHKTS | 963 |
| SPHKTT | 964 |
| SPHKTY | 965 |
| SPHKYG | 966 |
| SPHSKD | 967 |
| SPHSKP | 968 |
| SPHSRA | 969 |
| SPHSSR | 970 |
| SPHWKA | 971 |
| SPHTRG | 972 |
| SPHVRG | 973 |
| SPHKRG | 974 |
| SPHGAR | 975 |
| SPHRSG | 976 |
| SPHKSA | 977 |
| SPHSKR | 978 |
| SPHFLR | 979 |
| SPHVRR | 980 |
| STHASR | 985 |
| SQHKSG | 986 |
| HDSPHK | 2 |
| HDSPHSKA | 4486 |

In some embodiments, the AAV capsid variant comprises (e.g., in loop IV) an amino acid sequence having the formula [N1]-1N21-1N31, wherein 1N21 comprises the amino acid sequence of SPH and [N3] comprises amino acids X4. X5, and X6, wherein at least one of X4. X5, or X6 is a basic amino acid, e.g., a K or R, In some embodiments, X4 of 1[N2] is K, In some embodiments, X5 of [N2] is K.

In some embodiments, the AAV capsid variant comprises an amino acid sequence having the formula IN 11-[N2]-[N3], wherein: [N1] comprises X1, X2, and X3, wherein at least one of X1, X2, or X3 is G: [N2] comprises the amino acid sequence of SPH; and [N3] comprises X4, X5, and X6, wherein at least one of X4, X5, or X6 is a basic amino acid, e.g., a K or R; wherein [N1]-[N2]-[N3] is present in hypervariable loop IV; and wherein the AAV capsid variant comprises an amino acid sequence at least 95% identical to the amino acid sequence corresponding to positions 203-736 of SEQ ID NO: 138.

In some embodiments, [N1] comprises X1, X2, and X3, wherein at least one of X1, X2, or X3 is G, In some embodiments, X1 of [N1] is G, V, R, D, E, M, T, I, S, A, N, L, K, H, P, W, or C, In some embodiments. X2 of [N1 j is: S, V, L, N, D, H, R, P, G, T, I, A, E, Y, M, or Q, In some embodiments, X3 of [N1] is: G, C, L, D, E, Y, H, V, A, N, P, or S, In some embodiments. [N1] comprises GS, SG, GH, HD, GQ, QD, VS, CS, GR, RG, QS, SH, MS, RN, TS, IS, GP, ES, SS, GN, AS, NS, LS, GG, KS, GT, PS, RS, GI, WS, DS, ID, GL, DA, DG, ME, EN, KN, KE, AI, NG, PG, TG, SV, IG, LG, AG, EG, SA, YD, HE, HG, RD, ND, PD, MG, QV, DD, HN, HP, GY, GM, GD, or HS. In some embodiments. [N1] comprises GS, SG, GH, or HD. In some embodiments [N1] is or comprises GSG, GHD, GQD, VSG, CSG, GRG, CSH, GQS. GSH, RVG, GSC, GLL, GDD, GHE, GNY, MSG, RNG, TSG, ISG, GPG, ESG, SSG, GNG, ASG, NSG, LSG, GGG, KSG, HSG, GTG, PSG, GSV, RSG, GIG, WSG, DSG, IDG, GLG, DAG, DGG, MEG, ENG, GSA, KNG, KEG, AIG, GYD, GHG, GRD, GND, GPD, GMG, GQV, GHN, GHP, or GHS. In some embodiments. [N1] is or comprises GSG. In some embodiments, [N1] is or comprises GHD. In some embodiments. [N1]-[N2] comprises SGSPH (SEQ ID NO: 4752). HDSPH (SEQ ID NO: 4703), QDSPH (SEQ ID NO: 4753). RGSPH (SEQ ID NO: 4754), SHSPH (SEQ ID NO: 4755), QSSPH (SEQ ID NO: 4756), DDSPH (SEQ ID NO: 4757). HESPH (SEQ ID NO: 4758). NYSPH (SEQ ID NO: 4759), VGSPH (SEQ ID NO: 4760), SCSPH (SEQ ID NO: 4761), LLSPH (SEQ ID NO: 4762). NGSPH (SEQ ID NO: 4763), PGSPH (SEQ ID NO: 4764), GGSPH (SEQ ID NO: 4765). TGSPH (SEQ ID NO: 4766), SVSPH (SEQ ID NO: 4767). IGSPH (SEQ ID NO: 4768), DGSPH (SEQ ID NO: 4769). LGSPH (SEQ ID NO: 4770), AGSPH (SEQ ID NO: 4771). EGSPH (SEQ ID NO: 4772), SASPH (SEQ ID NO: 4773), YDSPH (SEQ ID NO: 4774), HGSPH (SEQ ID NO: 4775). RDSPH (SEQ ID NO: 4776), NDSPH (SEQ ID NO: 4777), PDSPH (SEQ ID NO: 4778), MGSPH (SEQ ID NO: 4779), QVSPH (SEQ ID NO: 4780), HNSPH (SEQ ID NO: 4781). HPSPH (SEQ ID NO: 4782), or HSSPH (SEQ ID NO: 4783); an amino acid sequence comprising any portion of any of the aforsaid amino acid sequences (e.g., any 2, 3, or 4 amino acids, e.g., consecutive amino acids) thereof: an amino acid sequence comprising at least one, at least two, or at least three, but no more than four modifications relative to any of the aforesaid amino acid sequences; or an amino acid sequence comprising at least one, at least two, or at least three, but no more than four different amino acids, relative to any one of the aforesaid amino acid sequences. In some embodiments, [N1 j-[N2] is or comprises GSGSPH (SEQ ID NO: 4695), GHDSPH (SEQ ID NO: 4784), GQDSPH (SEQ ID NO: 4785). VSGSPH (SEQ ID NO: 4786), CSGSPH (SEQ ID NO: 4787), GRGSPH (SEQ ID NO: 4788), CSHSPH (SEQ ID NO: 4789), GQSSPH (SEQ ID NO: 4790), GSHSPH (SEQ ID NO: 4791), GDDSPH (SEQ ID NO: 4792), GHESPH (SEQ ID NO: 4793), GNYSPH (SEQ ID NO: 4794), RVGSPH (SEQ ID NO: 4795), GSCSPH (SEQ ID NO: 47%), GLLSPH (SEQ ID NO: 4797), MSGSPH (SEQ ID NO: 4798), RNGSPH (SEQ ID NO: 4799), TSGSPH (SEQ ID NO: 4800), ISGSPH (SEQ ID NO: 4801), GPGSPH (SEQ ID NO: 4802), ESGSPH (SEQ ID NO: 4803), SSGSPH (SEQ ID NO: 4804), GNGSPH (SEQ ID NO: 4805), ASGSPH (SEQ ID NO: 4806), NSGSPH (SEQ ID NO: 4807), LSGSPH (SEQ ID NO: 4808), GGGSPH (SEQ ID NO: 4809), KSGSPH (SEQ ID NO: 4810), HSGSPH (SEQ ID NO: 4811), GTGSPH (SEQ ID NO: 4812), PSGSPH (SEQ ID NO: 4813), GSVSPH (SEQ ID NO: 4814), RSGSPH (SEQ ID NO: 4815), GIGSPH (SEQ ID NO: 4816), WSGSPH (SEQ ID NO: 4817), DSGSPH (SEQ ID NO: 4818), IDGSPH (SEQ ID NO: 4819), GLGSPH (SEQ ID NO: 4820), DAGSPH (SEQ ID NO: 4821), DGGSPH (SEQ ID NO: 4822), MEGSPH (SEQ ID NO: 4823), ENGSPH (SEQ ID NO: 4824), GSASPH (SEQ ID NO: 4825), KNGSPH (SEQ ID NO: 4826), KEGSPH (SEQ ID NO: 4827), AIGSPH (SEQ ID NO: 4828), GYDSPH (SEQ ID NO: 4829), GHGSPH (SEQ ID NO: 4830), GRDSPH (SEQ ID NO: 4831), GNDSPH (SEQ ID NO: 4832), GPDSPH (SEQ ID NO: 4833), GMGSPH (SEQ ID NO: 4834), GQVSPH (SEQ ID NO: 4835), GHNSPH (SEQ ID NO: 4836), GHPSPH (SEQ ID NO: 4837), or GHSSPH (SEQ ID NO: 4838); an amino acid sequence comprising any portion of any of the aforesaid amino acid sequences (e.g., any 2, 3, 4, or 5 amino acids, e.g., consecutive amino acids) thereof; an amino acid sequence comprising one, two, or three but no more than four modifications relative to an of the aforesaid amino acid sequences; or an amino acid sequence comprising one, two, or three but no more than four different amino acids, relative to any one of the aforesaid amino acid sequences. In some embodiments. [N1]-[N2] is or comprises GSGSPH (SEQ ID NO: 4695). In some embodiments. [N1]-[N2] is or comprises GHDSPH (SEQ ID NO: 4784).

In some embodiments, X4, X5, or both of [N3] are K, In some embodiments, X4, X5, or X6 of [N3] is R, In some embodiments. X4 of [N3] is: A, K, V, S, T, G, F, W, V, N, or R, In some embodiments, X5 of [N3] is: S, K, T, F, I, L, Y, H, M, or R, In some embodiments, X6 of [N3] is: G, R, A, M, I, N, T, Y, D, P, V, L, E, W, N, Q, K, or S, In some embodiments, [N3] comprises SK, KA, KS, AR, RM, VK, AS, SR, VK, KR, KK, KN, VR, RS, RK, KT, TS, KF, FG, KI, IG, KL, LG, TT, TY, KY, YG, KD, KP, TR, RG, VR, GA, SL, SS, FL, WK, SA, RA, LR, KW, RR, GK, TK, NK, AK, KV, KG, KH, KM, TG, SE, SV, SW, SN, HG, SQ, LW, MG, MA, or SG. In some embodiments. [N3] comprises SK, KA, KS, or SG. In some embodiments, [N3] is or comprises SKA, KSG, ARM. VKS, ASR, VKI, KKN, VRM, RKA, KTS, KFG, KIG, KLG, KTT, KTY, KYG, SKD, SKP, TRG, VRG, KRG, GAR, KSA, KSR, SKL, SRA, SKR, SLR, SRG, SSR, FLR, SKW, SKS, WKA, VRR, SKV, SKT, SKG, GKA, TKA, NKA, SKL, SKN, AKA, KTG, KSL, KSE, KSV, KSW, KSN, KHG, KSQ, KSK, KLW, WKG, KMG, KMA, or RSG. In some embodiments. [N3] is or comprises SKA. In some embodiments, [N3] is or comprises KSG. In some embodiments. [N2]-[N3] comprises SPHSK (SEQ ID NO: 4701), SPHKS (SEQ ID NO: 4704), SPHAR (SEQ ID NO: 4705), SPHVK (SEQ ID NO: 4706), SPHAS (SEQ ID NO: 4707), SPHKK (SEQ ID NO: 4708), SPHVR (SEQ ID NO: 4709), SPHRK (SEQ ID NO: 4710), SPHKT (SEQ ID NO: 4711), SPHKF (SEQ ID NO: 4712). SPHKI (SEQ ID NO: 4713), SPHKL (SEQ ID NO: 4714), SPHKY (SEQ ID NO: 4715), SPHTR (SEQ ID NO: 4716), SPHKR (SEQ ID NO: 4717), SPHGA (SEQ ID NO: 4718), SPHSR (SEQ ID NO: 4719), SPHSL (SEQ ID NO: 4720), SPHSS (SEQ ID NO: 4721), SPHFL (SEQ ID NO: 4722), SPHWK (SEQ ID NO: 4723), SPHGK (SEQ ID NO: 4724), SPHTK (SEQ ID NO: 4725), SPHNK (SEQ ID NO: 4726), SPHAK (SEQ ID NO: 4727), SPHKH (SEQ ID NO: 4728), SPHKM (SEQ ID NO: 4729), or SPHRS (SEQ ID NO: 4730). In some embodiments [N2]-[N3] comprises SPHSK (SEQ ID NO: 4701) or SPHKS (SEQ ID NO: 4704). In some embodiments. [N2]-[N3] is or comprises SPHSKA (SEQ ID NO: 941), SPHKSG (SEQ ID NO: 946), SPHARM (SEQ ID NO: 947). SPHVKS (SEQ ID NO: 948), SPHASR (SEQ ID NO: 949), SPHVKI (SEQ ID NO: 950), SPHKKN (SEQ ID NO: 954), SPHVRM (SEQ ID NO: 955), SPHRKA (SEQ ID NO: 956), SPHKFG (SEQ ID NO: 957), SPHKIG (SEQ ID NO: 958), SPHKLG (SEQ ID NO: 959), SPHKTS (SEQ ID NO: 963). SPHKTT (SEQ ID NO: 9%4), SPHKTY (SEQ ID NO: 9%5), SPHKYG (SEQ ID NO: 966), SPHSKD (SEQ ID NO: 97), SPHSKP (SEQ ID NO: %8), SPHTRG (SEQ ID NO: 972), SPHVRG (SEQ ID NO: 973), SPHKRG (SEQ ID NO: 974), SPHGAR (SEQ ID NO: 975), SPHKSA (SEQ ID NO: 977). SPHKSR (SEQ ID NO: 951), SPHSKL (SEQ ID NO: 960), SPHSRA (SEQ ID NO: 969), SPHSKR (SEQ ID NO: 978), SPHSLR (SEQ ID NO: 952), SPHSRG (SEQ ID NO: 961), SPHSSR (SEQ ID NO: 970), SPHFLR (SEQ ID NO: 979), SPHSKW (SEQ ID NO: 953), SPHSKS (SEQ ID NO: 962). SPHWKA (SEQ ID NO: 971), SPHVRR (SEQ ID NO: 980), SPHSKT (SEQ ID NO: 4731), SPHSKG (SEQ ID NO: 4732), SPHGKA (SEQ ID NO: 4733), SPHNKA (SEQ ID NO: 4734), SPHSKN (SEQ ID NO: 4735), SPHAKA (SEQ ID NO: 4736), SPHSKV (SEQ ID NO: 4737), SPHKTG (SEQ ID NO: 4738), SPHTKA (SEQ ID NO: 4739), SPHKSL (SEQ ID NO: 4740), SPHKSE (SEQ ID NO: 4741), SPHKSV (SEQ ID NO: 4742), SPHKSW (SEQ ID NO: 4743), SPHKSN (SEQ ID NO: 4744), SPHKHG (SEQ ID NO: 4745), SPHKSQ (SEQ ID NO: 4746), SPHKSK (SEQ ID NO: 4747), SPHKLW (SEQ ID NO: 4748), SPHWKG (SEQ ID NO: 4749), SPHKMG (SEQ ID NO: 4750), SPHKMA (SEQ ID NO: 4751), or SPHRSG (SEQ ID NO: 976). In some embodiments, [N2]-[N3] is or comprises SPHSKA (SEQ ID NO: 941). In some embodiments, [N2]-[N3] is or comprises SPHKSG (SEQ ID NO: 946).

In some embodiments, [N1]-[N2]-[N3] comprises SGSPHSK (SEQ ID NO: 4839), HDSPHKS (SEQ ID NO: 4840), SGSPHAR (SEQ ID NO: 4841), SGSPHVK (SEQ ID NO: 4842), QDSPHKS (SEQ ID NO: 4843), SGSPHKK (SEQ ID NO: 4844), SGSPHVR (SEQ ID NO: 4845), SGSPHAS (SEQ ID NO: 4846), SGSPHRK (SEQ ID NO: 4847), SGSPHKT (SEQ ID NO: 4848), SHSPHKS (SEQ ID NO: 4849), QSSPHRS (SEQ ID NO: 4850), RGSPHAS (SEQ ID NO: 4851), RGSPHSK (SEQ ID NO: 4852), SGSPHKF (SEQ ID NO: 4853), SGSPHKI (SEQ ID NO: 4854), SGSPHKL (SEQ ID NO: 4855), SGSPHKY (SEQ ID NO: 4856), SGSPHTR (SEQ ID NO: 4857), SHSPHKR (SEQ ID NO: 4858), SGSPHGA (SEQ ID NO: 4859), HDSPHKR (SEQ ID NO: 4860), DDSPHKS (SEQ ID NO: 4861), HESPHKS (SEQ ID NO: 4862), NYSPHKI (SEQ ID NO: 4863), SGSPHSR (SEQ ID NO: 4864), SGSPHSL (SEQ ID NO: 4865), SGSPHSS (SEQ ID NO: 4866), VGSPHSK (SEQ ID NO: 4867), SCSPHRK (SEQ ID NO: 4868), SGSPHFL (SEQ ID NO: 4869), LLSPHWK (SEQ ID NO: 4870), NGSPHSK (SEQ ID NO: 4871), PGSPHSK (SEQ ID NO: 4872), GGSPHSK (SEQ ID NO: 4873), TGSPHSK (SEQ ID NO: 4874), SVSPHGK (SEQ ID NO: 4875), SGSPHTK (SEQ ID NO: 4876), IGSPHSK (SEQ ID NO: 4877), DGSPHSK (SEQ ID NO: 4878), SGSPHNK (SEQ ID NO: 4879), LGSPHSK (SEQ ID NO: 4880), AGSPHSK (SEQ ID NO: 4881), EGSPHSK (SEQ ID NO: 4882), SASPHSK (SEQ ID NO: 4883), SGSPHAK (SEQ ID NO: 4884), HDSPHKI (SEQ ID NO: 4885), YDSPHKS (SEQ ID NO: 4886), HDSPHKT (SEQ ID NO: 4887), RGSPHKR (SEQ ID NO: 4888), HGSPHSK (SEQ ID NO: 4889), RDSPHKS (SEQ ID NO: 4890), NDSPHKS (SEQ ID NO: 4891), QDSPHKI (SEQ ID NO: 4892), PDSPHKI (SEQ ID NO: 4893), PDSPHKS (SEQ ID NO: 4894), MGSPHSK (SEQ ID NO: 4895), HDSPHKH (SEQ ID NO: 48%), QVSPHKS (SEQ ID NO: 4897), HNSPHKS (SEQ ID NO: 4898), NGSPHKR (SEQ ID NO: 4899), HDSPHKY (SEQ ID NO: 4900), NDSPHKI (SEQ ID NO: 4901), HDSPHKL (SEQ ID NO: 4902), HPSPHWK (SEQ ID NO: 4903), HDSPHKM (SEQ ID NO: 4904), orHSSPHRS (SEQ ID NO: 4905). In some embodiments. [N1]-[N2]-[N3] is GSGSPHSKA (SEQ ID NO: 4697). GHDSPHKSG (SEQ ID NO: 4698), GSGSPHARM (SEQ ID NO: 4906). GSGSPHVKS (SEQ ID NO: 4907), GQDSPHKSG (SEQ ID NO: 4908), GSGSPHASR (SEQ ID NO: 4909), GSGSPHVKI (SEQ ID NO: 4910), GSGSPHKKN (SEQ ID NO: 4911), GSGSPHVRM (SEQ ID NO: 4912), VSGSPHSKA (SEQ ID NO: 4913), CSGSPHSKA (SEQ ID NO: 4914), GSGSPHRKA (SEQ ID NO: 4915), CSGSPHKTS (SEQ ID NO: 4916). CSHSPHKSG (SEQ ID NO: 4917), GQSSPHRSG (SEQ ID NO: 4918), GRGSPHASR (SEQ ID NO: 4919). GRGSPHSKA (SEQ ID NO: 4920), GSGSPHKFG (SEQ ID NO: 4921), GSGSPHKIG (SEQ ID NO: 4922), GSGSPHKLG (SEQ ID NO: 4923), GSGSPHKTS (SEQ ID NO: 4924), GSGSPHKTT (SEQ ID NO: 4925), GSGSPHKTY (SEQ ID NO: 4926), GSGSPHKYG (SEQ ID NO: 4927), GSGSPHSKD (SEQ ID NO: 4928), GSGSPHSKP (SEQ ID NO: 4929), GSGSPHTRG (SEQ ID NO: 4930). GSGSPHVRG (SEQ ID NO: 4931), GSHSPHKRG (SEQ ID NO: 4932), GSHSPHKSG (SEQ ID NO: 4933), VSGSPHASR (SEQ ID NO: 4934), VSGSPHGAR (SEQ ID NO: 4935), VSGSPHKFG (SEQ ID NO: 4936), GHDSPHKRG (SEQ ID NO: 4937), GDDSPHKSG (SEQ ID NO: 4938). GHESPHKSA (SEQ ID NO: 4939), GHDSPHKSA (SEQ ID NO: 4940), GNYSPHKIG (SEQ ID NO: 4941). GHDSPHKSR (SEQ ID NO: 4942). GSGSPHSKL (SEQ ID NO: 4943), GSGSPHSRA (SEQ ID NO: 4944), GSGSPHSKR (SEQ ID NO: 4945), GSGSPHSLR (SEQ ID NO: 4946). GSGSPHSRG (SEQ ID NO: 4947), GSGSPHSSR (SEQ ID NO: 4948), RVGSPHSKA (SEQ ID NO: 4949), GSCSPHRKA (SEQ ID NO: 4950), GSGSPHFLR (SEQ ID NO: 4951), GSGSPHSKW (SEQ ID NO: 4952), GSGSPHSKS (SEQ ID NO: 4953), GLLSPHWKA (SEQ ID NO: 4954). GSGSPHVRR (SEQ ID NO: 4955), GSGSPHSKV (SEQ ID NO: 4956), MSGSPHSKA (SEQ ID NO: 4957), RNGSPHSKA (SEQ ID NO: 4958), TSGSPHSKA (SEQ ID NO: 4959), ISGSPHSKA (SEQ ID NO: 4960), GPGSPHSKA (SEQ ID NO: 4%1), GSGSPHSKT (SEQ ID NO: 4962), ESGSPHSKA (SEQ ID NO: 4963), SSGSPHSKA (SEQ ID NO: 4964), GNGSPHSKA (SEQ ID NO: 4965), ASGSPHSKA (SEQ ID NO: 4966), NSGSPHSKA (SEQ ID NO: 49%7), LSGSPHSKA (SEQ ID NO: 4968), GGGSPHSKA (SEQ ID NO: 4969), KSGSPHSKA (SEQ ID NO: 4970). GGGSPHSKS (SEQ ID NO: 4971). GSGSPHSKG (SEQ ID NO: 4972), HSGSPHSKA (SEQ ID NO: 4973), GTGSPHSKA (SEQ ID NO: 4974), PSGSPHSKA (SEQ ID NO: 4975), GSVSPHGKA (SEQ ID NO: 4976). RSGSPHSKA (SEQ ID NO: 4977), GSGSPHTKA (SEQ ID NO: 4978), GIGSPHSKA (SEQ ID NO: 4979). WSGSPHSKA (SEQ ID NO: 4980), DSGSPHSKA (SEQ ID NO: 4981), IDGSPHSKA (SEQ ID NO: 4982), GSGSPHNKA (SEQ ID NO: 4983), GLGSPHSKS (SEQ ID NO: 4984), DAGSPHSKA (SEQ ID NO: 4985), DGGSPHSKA (SEQ ID NO: 4986), MEGSPHSKA (SEQ ID NO: 4987), ENGSPHSKA (SEQ ID NO: 4988), GSASPHSKA (SEQ ID NO: 4989), GNGSPHSKS (SEQ ID NO: 4990), KNGSPHSKA (SEQ ID NO: 4991), KEGSPHSKA (SEQ ID NO: 4992), AIGSPHSKA (SEQ ID NO: 4993), GSGSPHSKN (SEQ ID NO: 4994), GSGSPHAKA (SEQ ID NO: 4995), GHDSPHKIG (SEQ ID NO: 4996), GYDSPHKSG (SEQ ID NO: 4997), GHESPHKSG (SEQ ID NO: 4998), GHDSPHKTG (SEQ ID NO: 4999), GRGSPHKRG (SEQ ID NO: 5000), GQDSPHKSG (SEQ ID NO: 4908), GHDSPHKSL (SEQ ID NO: 5001), GHGSPHSKA (SEQ ID NO: 5002), GHDSPHKSE (SEQ ID NO: 5003), VSGSPHSKA (SEQ ID NO: 4913), GRDSPHKSG (SEQ ID NO: 5004), GNDSPHKSV (SEQ ID NO: 5005), GQDSPHKIG (SEQ ID NO: 5006), GHDSPHKSV (SEQ ID NO: 5007), GPDSPHKIG (SEQ ID NO: 5008), GPDSPHKSG (SEQ ID NO: 5009), GHDSPHKSW (SEQ ID NO: 5010), GHDSPHKSN (SEQ ID NO: 5011), GMGSPHSKT (SEQ ID NO: 5012), GHDSPHKHG (SEQ ID NO: 5013), GQVSPHKSG (SEQ ID NO: 5014), GDDSPHKSV (SEQ ID NO: 5015), GHNSPHKSG (SEQ ID NO: 5016), GNGSPHKRG (SEQ ID NO: 5017), GHDSPHKYG (SEQ ID NO: 5018), GHDSPHKSQ (SEQ ID NO: 5019), GNDSPHKIG (SEQ ID NO: 5020), GHDSPHKSK (SEQ ID NO: 5021), GHDSPHKLW (SEQ ID NO: 5022). GHPSPHWKG (SEQ ID NO: 5023), GHDSPHKMG (SEQ ID NO: 5024), GHDSPHKMA (SEQ ID NO: 5025), or GHSSPHRSG (SEQ ID NO: 5026): an amino acid sequence comprising any portion of any of the aforesaid amino acid sequences (e.g., any 2, 3, 4, 5, 6, 7, or 8 amino acids, e.g., consecutive amino acids) thereof: an amino acid sequence comprising one, two, or three but no more than four modifications relative to any of the aforesaid amino acid sequences: or an amino acid sequence comprising one, two, or three but no more than four different amino acids, relative to any one of the aforesaid amino acid sequences. In some embodiments, [N1]-[N2]-[N3] is or comprises GSGSPHSKA (SEQ ID NO: 4697). In some embodiments. [N1]-[N2]-[N3] is or comprises GHDSPHKSG (SEQ ID NO: 4698).

In some embodiments, the AAV capsid variant comprising an amino acid sequence having the formula [N1-[N2]-[N3] (e.g., in loop IV) further comprises [N4], which comprises X7 X8 X9 X10. In some embodiments, X7 of [N4] is W, Q, K, R, G, L, V, S, P, H, K, 1, M, A, E, or F, In some embodiments, X8 of [N4] is N, Y, C, K, T, H, R, D, V, S, P, G, W, E, F, A, 1, M, Q, or L, In some embodiments. X9 of [N4] is Q, G, K, H, R, T, L, D, A, P1, F, V, M, W, Y, S, E, N, or Y, In some embodiments. X10 of [N4] is Q, H, L, R, W, K, A, P, E, M, S, G, N, Y, C, V, T, D, or V, In some embodiments [N4] is or comprises QNQQ (SEQ ID NO: 5028), WNQQ (SEQ ID NO: 5029), QYYV (SEQ ID NO: 5030), RRQQ (SEQ ID NO: 5031), QNQQ (SEQ ID NO: 5028), GCGQ (SEQ ID NO: 5032). LRQQ (SEQ ID NO: 5033), RNQQ (SEQ ID NO: 5034). VNQQ (SEQ ID NO: 5035), FRLQ (SEQ ID NO: 5036), FNQQ (SEQ ID NO: 5037), LLQQ (SEQ ID NO: 5038), SNQQ (SEQ ID NO: 5039), RLQQ (SEQ ID NO: 5040), LNQQ (SEQ ID NO: 5041), QRKL (SEQ ID NO: 5042). LRRQ (SEQ ID NO: 5043), QRLR (SEQ ID NO: 5044), QRRL (SEQ ID NO: 5045), RRLQ (SEQ ID NO: 5046), RLRQ (SEQ ID NO: 5047), SKRQ (SEQ ID NO: 5048), QLYR (SEQ ID NO: 5049), QLTV (SEQ ID NO: 5050), QNKQ (SEQ ID NO: 5051), KNQQ (SEQ ID NO: 5052), QKQQ (SEQ ID NO: 5053), QTQQ (SEQ ID NO: 5054), QNHQ (SEQ ID NO: 5055), QHQQ (SEQ ID NO: 5056), QNQH (SEQ ID NO: 5057), QHRQ (SEQ ID NO: 5058), LTQQ (SEQ ID NO: 5059), QNQW (SEQ ID NO: 5060), QNTH (SEQ ID NO: 5061), RRRQ (SEQ ID NO: 5062), QYQQ (SEQ ID NO: 5063), QNDQ (SEQ ID NO: 5064), QNRH (SEQ ID NO: 5065), RDQQ (SEQ ID NO: 5066), PNLQ (SEQ ID NO: 5067), HVRQ (SEQ ID NO: 5068), PNQH (SEQ ID NO: 5069), HNQQ (SEQ ID NO: 5070), QSQQ (SEQ ID NO: 5071), QPAK (SEQ ID NO: 5072), QNLA (SEQ ID NO: 5073), QNQL (SEQ ID NO: 5074), QGQQ (SEQ ID NO: 5075), LNRQ (SEQ ID NO: 5076), QNPP (SEQ ID NO: 5077), QNLQ (SEQ ID NO: 5078), QDQE (SEQ ID NO: 5079), QDQQ (SEQ ID NO: 5080), HWQQ (SEQ ID NO: 5081), PNQQ (SEQ ID NO: 5082), PEQQ (SEQ ID NO: 5083), QRTM (SEQ ID NO: 5084), LHQH (SEQ ID NO: 5085), QHRI (SEQ ID NO: 5086), QYIH (SEQ ID NO: 5087), QKFE (SEQ ID NO: 5088), QFPS (SEQ ID NO: 5089), QNPL (SEQ ID NO: 5090), QAIK (SEQ ID NO: 5091), QNRQ (SEQ ID NO: 5092), QYQH (SEQ ID NO: 5093), QNPQ (SEQ ID NO: 5094), QHQL (SEQ ID NO: 5095), QSPP (SEQ ID NO: 5096), QAKL (SEQ ID NO: 5097), KSQQ (SEQ ID NO: 5098), QDRP (SEQ ID NO: 5099), QNLG (SEQ ID NO: 5100), QAFH (SEQ ID NO: 5101), QNAQ (SEQ ID NO: 5102), HNQL (SEQ ID NO: 5103), QKLN (SEQ ID NO: 5104), QNVQ (SEQ ID NO: 5105), QAQQ (SEQ ID NO: 5106), QTPP (SEQ ID NO: 5107), QPPA (SEQ ID NO: 5108), QERP (SEQ ID NO: 5109), QDLQ (SEQ ID NO: 5110), QAMH (SEQ ID NO: 5111), QHPS (SEQ ID NO: 5112), PGLQ (SEQ ID NO: 5113), QGIR (SEQ ID NO: 5114), QAPA (SEQ ID NO: 5115), QIPP (SEQ ID NO: 5116), QTQL (SEQ ID NO: 5117), QAPS (SEQ ID NO: 5118), QNTY (SEQ ID NO: 5119), QDKQ (SEQ ID NO: 5120), QNHL (SEQ ID NO: 5121), QIGM (SEQ ID NO: 5122), LNKQ (SEQ ID NO: 5123), PNQL (SEQ ID NO: 5124), QLQQ (SEQ ID NO: 5125), QRMS (SEQ ID NO: 5126), QGIL (SEQ ID NO: 5127), QDRQ (SEQ ID NO: 5128), RDWQ (SEQ ID NO: 5129), QERS (SEQ ID NO: 5130), QNYQ (SEQ ID NO: 5131), QRTC (SEQ ID NO: 5132), QIGH (SEQ ID NO: 5133), QGAI (SEQ ID NO: 5134), QVPP (SEQ ID NO: 5135), QVQQ (SEQ ID NO: 5136), LMRQ (SEQ ID NO: 5137), QYSV (SEQ ID NO: 5138), QAIT (SEQ ID NO: 5139), QKTL (SEQ ID NO: 5140), QLHH (SEQ ID NO: 5141), QNII (SEQ ID NO: 5142), QGHH (SEQ ID NO: 5143), QSKV (SEQ ID NO: 5144), QLPS (SEQ ID NO: 5145), IGKQ (SEQ ID NO: 5146), QAIH (SEQ ID NO: 5147), QHGL (SEQ ID NO: 5148), QFMC (SEQ ID NO: 5149), QNQM (SEQ ID NO: 5150), QHLQ (SEQ ID NO: 5151), QPAR (SEQ ID NO: 5152), QSLQ (SEQ ID NO: 5153), QSQL (SEQ ID NO: 5154), HSQQ (SEQ ID NO: 5155), QMPS (SEQ ID NO: 5156), QGSL (SEQ ID NO: 5157), QVPA (SEQ ID NO: 5158), HYQQ (SEQ ID NO: 5159), QVPS (SEQ ID NO: 5160), RGEQ (SEQ ID NO: 5161), PGQQ (SEQ ID NO: 5162), LEQQ (SEQ ID NO: 5163), QNQS (SEQ ID NO: 5164), QKVI (SEQ ID NO: 5165), QNND (SEQ ID NO: 5166), QSVH (SEQ ID NO: 5167), QPLG (SEQ ID NO: 5168), HNQE (SEQ ID NO: 5169), QIQQ (SEQ ID NO: 5170), QVRN (SEQ ID NO: 5171), PSNQ (SEQ ID NO: 5172), QVGH (SEQ ID NO: 5173), QRDI (SEQ ID NO: 5174), QMPN (SEQ ID NO: 5175), RGLQ (SEQ ID NO: 5176), PSLQ (SEQ ID NO: 5177), QRDQ (SEQ ID NO: 5178), QAKG (SEQ ID NO: 5179), QSAH (SEQ ID NO: 5180), QSTM (SEQ ID NO: 5181), QREM (SEQ ID NO: 5182), QYRA (SEQ ID NO: 5183), QRQQ (SEQ ID NO: 5184), QWQQ (SEQ ID NO: 5185), QRMN (SEQ ID NO: 5186), GDSQ (SEQ ID NO: 5187), QKIS (SEQ ID NO: 5188), PSMQ (SEQ ID NO: 5189), SPRQ (SEQ ID NO: 5190), MEQQ (SEQ ID NO: 5191), QYQN (SEQ ID NO: 5192), QIRQ (SEQ ID NO: 5193), QSVQ (SEQ ID NO: 5194), RSQQ (SEQ ID NO: 5195), QNKL (SEQ ID NO: 5196), QIQH (SEQ ID NO: 5197). PRQQ (SEQ ID NO: 5198), HTQQ (SEQ ID NO: 5199), QRQH (SEQ ID NO: 5200). RNQE (SEQ ID NO: 5201), QSKQ (SEQ ID NO: 5202), QNQP (SEQ ID NO: 5203), QSPQ (SEQ ID NO: 5204), QTRQ (SEQ ID NO: 5205), QNLH (SEQ ID NO: 5206), QNQE (SEQ ID NO: 5207). LNQP (SEQ ID NO: 5208), QNQD (SEQ ID NO: 5209), QNLL (SEQ ID NO: 5210), QLVI (SEQ ID NO: 5211), RTQE (SEQ ID NO: 5212), QTHQ (SEQ ID NO: 5213), QDQH (SEQ ID NO: 5214), QSQH (SEQ ID NO: 5215), VRQQ (SEQ ID NO: 5216), AWQQ (SEQ ID NO: 5217), QSVP (SEQ ID NO: 5218), QNIQ (SEQ ID NO: 5219), LDQQ (SEQ ID NO: 5220), PDQQ (SEQ ID NO: 5221), ESQQ (SEQ ID NO: 5222), QRQL (SEQ ID NO: 5223), Q1IV (SEQ ID NO: 5224). QKQS (SEQ ID NO: 5225), QSHQ (SEQ ID NO: 5226), QFVV (SEQ ID NO: 5227), QSQP (SEQ ID NO: 5228), QNEQ (SEQ ID NO: 5229), INQQ (SEQ ID NO: 5230), RNRQ (SEQ ID NO: 5231), RDQK (SEQ ID NO: 5232), QWKR (SEQ ID NO: 5233), ENRQ (SEQ ID NO: 5234), QTQP (SEQ ID NO: 5235), QKQL (SEQ ID NO: 5236), RNQL (SEQ ID NO: 5237), ISIQ (SEQ ID NO: 5238). QTVC (SEQ ID NO: 5239), QQIM (SEQ ID NO: 5240). LNHQ (SEQ ID NO: 5241), QNQA (SEQ ID NO: 5242), QMIH (SEQ ID NO: 5243), RNHQ (SEQ ID NO: 5244), or QKMN (SEQ ID NO: 5245), or any dipeptide or tripeptide thereof. In some embodiments. [N1]-[N2]-[N3]-[N4] is or comprises: the amino acid sequence of any of SEQ ID NOs: 1800-2241; an amino acid sequence comprising any portion of any of the aforesaid amino acid sequences (e.g., any 2, 3, 4, 5, 6, 7, 8, 9, 10, 11, or 12 amino acids, e.g., consecutive amino acids) thereof; an amino acid sequence comprising one, two, or three but no more than four modifications relative to any of the aforesaid amino acid sequences; or an amino acid sequence comprising one, two, or three but no more than four different amino acids, relative to any one of the aforesaid amino acid sequences. In some embodiments, [N1]-[N2]-[N3]-[N4] is or comprises GSGSPHSKAQNQQ (SEQ ID NO: 1801). In some embodiments, [N1]-[N2]-[N3]-[N4] is or comprises GHDSPHKSGQNQQ (SEQ ID NO: 1800).

In some embodiments, the AAV capsid variant comprises (e.g., in loop IV) an amino acid sequence having the formula [N1]-[N2]-[N3]; and further comprises [N0], which comprises X_{A} Xj and X_{C}. In some embodiments. X_{A} of [N0] is T, S, Y, M, A, C, I, R, L, D, F, V, Q, N, H, E, or G, In some embodiments, X₃ of [N0] is 1. M, P, E, N, D, S, A, T, G, Q, F, V, L, C, H, R, W, or L, In some embodiments, Xc of [N0] is N, M, E, G, Y, W, T, I, Q, F, V, A, L, I, P, K, R, H, S, D, or S, In some embodiments, [N0] is or comprises TIN, SMN, TIM, YLS, GLS, MPE, MEG, MEY, AEW, CEW, ANN, IPE, ADM, IEY, ADY, IET, MEW, CEY, RIN, MEI, LEY, ADW, IEI, DIM, FEQ, MEF, CDQ, LPE, IEN, MES, AEI, VEY, IIN, TSN, IEV, MEM, AEV, MDA, VEW, AEQ, LEW, MEL, MET, MEA, IES, MEV, CEI, ATN, MDG, QEV, ADQ, NMN, IEM, ISN, TGN, QQQ, HDW, IEG, TiH, TFP, TEK, EIN, TVN, TFN, SIN, TER, TSY, ELH, AlN, SVN, TDN, TFH, TVH, TEN, TSS, TID, TCN, NIN, TEH, AEM, AIK, TDK, TFK, SDQ, TEI, NTN, TET, SIK, TEL, TEA, TAN, TIY, TFS, TES, TTN, TED, TNN, EVH, TIS, TVR, TDR, TIK, NHI, TIP, ESD, TDL, TVP, TVI, AEH, NCL, TVK, NAD, TIT, NCV, TIR, NAL, VIN, TIQ, TEF, TRE, QGE, SEK, NVN, GGE, EFV, SDK, TEQ, EVQ, TEY, NCW, TDV, SDI, NSI, NSL, EVV, TEP, SEL, TWQ, TEV, AVN, GVL, TLN, TEG, TRD, NAT, AEN, AET, ETA, NNL, or any dipeptide thereof. In some embodiments. [N0]-[N1]-[N2]-[N3]-[N4] is or comprises the amino acid sequence of any one of SEQ ID NOs: 2242-2886: an amino acid sequence comprising any portion of any of the aforesaid amino acid sequences (e.g., any 2, 3, 4, 5, 6, 7, 8, 9, 10, 11, 12, 13, 14, or 15 amino acids, e.g., consecutive amino acids) thereof; an amino acid sequence comprising one, two, or three but no more than four modifications relative to any of the aforesaid amino acid sequences; or an amino acid sequence comprising one, two, or three but no more than four different amino acids, relative to any one of the aforesaid amino acid sequences. In some embodiments. [N0]-[N1]-[N2]-[N3]-[N4] is or comprises TINGSGSPHSKAQNQQ (SEQ ID NO: 2242). In some embodiments, [N0]-[N1J-[N2]-[N3]-[N4j is or comprises TINGHDSPHKSGQNQQ (SEQ ID NO: 2243).

In some embodiments. [N3] is present immediately subsequent to [N2]. In some embodiments, the amino acid sequence comprises, from N-terminus to C-terminus. [N2]-[N31. In some embodiments, the amino acid sequence comprises, from N-terminus to C-terminus, [N1]-[N2]-[N31. In some embodiments, the amino acid sequence comprises, from N-terminus to C-terminus. [N1]-[N2]-[N3]-[N4]. In some embodiments, the amino acid sequence comprises, from N-terminus to C-terminus, [N0]-[N1]-[N2]-[N3]. In some embodiments, the amino acid sequence comprises, from N-terminus to C-terminus, [N0]-[N1]-[N2]-[N3J-[N4].

In some embodiments, the AAV capsid variant comprises (e.g., in loop IV) an amino acid sequence having the formula [A][B] (SEQ ID NO: 4694), wherein [A] comprises the amino acid sequence of GSGSPH (SEQ ID NO: 4695) and [B] comprises X1 X2 X3 X4 X5 X6 X7. In some embodiments, X1 of [B] is S, C, F, or V, In some embodiments, X2 of [B] is K, L, R, I, E, Y, V, or S. In some embodiments. X3 of [B] is A, R, L, G, I, Y, S, F, or W, In some embodiments X4 of [B] is W, Q, R, G, L, V, S, or F, In some embodiments. X5 of [B] is N, Y, R, C, K, or L, In some embodiments. X6 of [B] is Q, G, K, R, T, L, or Y, In some embodiment, X7 of [B] is Q, L, R, or V. In some embodiments. [B] comprises SLLWNQQ (SEQ ID NO: 5247), SKAQYYV (SEQ ID NO: 5248), SKLRRQQ (SEQ ID NO: 5249), SIWQNQQ (SEQ ID NO: 5250), SKAGCGQ (SEQ ID NO: 5251), SRAQNQQ (SEQ ID NO: 5252), SKRLRQQ (SEQ ID NO: 5253), SLRRNQQ (SEQ ID NO: 5254), SRGRNQQ (SEQ ID NO: 5255), SEIVNQQ (SEQ ID NO: 5256), SSRRNQQ (SEQ ID NO: 5257), CLLQNQQ (SEQ ID NO: 5258), SKAFRLQ (SEQ ID NO: 5259), CLAQNQQ (SEQ ID NO: 5260), FLRQNQQ (SEQ ID NO: 5261), SLRFNQQ (SEQ ID NO: 5262), SYLRNQQ (SEQ ID NO: 5263), CSLQNQQ (SEQ ID NO: 5264), VLWQNQQ (SEQ ID NO: 5265), SKWLLQQ (SEQ ID NO: 5266), SLWSNQQ (SEQ ID NO: 5267), SKRRLQQ (SEQ ID NO: 5268), SVYLNQQ (SEQ ID NO: 5269), SLWLNQQ (SEQ ID NO: 5270), SKAQRKL (SEQ ID NO: 5271), SKALRRQ (SEQ ID NO: 5272), SKAQRLR (SEQ ID NO: 5273), SKAQNQQ (SEQ ID NO: 5274), SKAQRRL (SEQ ID NO: 5275), SKARRQQ (SEQ ID NO: 5276), SKARRLQ (SEQ ID NO: 5277), SKSRRQQ (SEQ ID NO: 5278), SKARLRQ (SEQ ID NO: 5279), SKASKRQ (SEQ ID NO: 5280). VRRQNQQ (SEQ ID NO: 5281), SKAQLYR (SEQ ID NO: 5282), SLFRNQQ (SEQ ID NO: 5283), SKAQLTV (SEQ ID NO: 5284), or any dipeptide, tripeptide, tetrapeptide, pentapeptide, or hexapeptide thereof. In some embodiments. [A][B] comprises GSGSPHSLLWNQQ (SEQ ID NO: 5285). GSGSPHSKAQYYV (SEQ ID NO: 2060). GSGSPHSKLRRQQ (SEQ ID NO: 2061), GSGSPHSIWQNQQ (SEQ ID NO: 5286), GSGSPHSKAGCGQ (SEQ ID NO: 2062), GSGSPHSRAQNQQ (SEQ ID NO: 2063). GSGSPHSKRLRQQ (SEQ ID NO: 2064), GSGSPHSLRRNQQ (SEQ ID NO: 2065), GSGSPHSRGRNQQ (SEQ ID NO: 2066), GSGSPHSEIVNQQ (SEQ ID NO: 5287), GSGSPHSSRRNQQ (SEQ ID NO: 2067), GSGSPHCLLQNQQ (SEQ ID NO: 5288), GSGSPHSKAFRLQ (SEQ ID NO: 2068), GSGSPHCLAQNQQ (SEQ ID NO: 5289), GSGSPHFLRQNQQ (SEQ ID NO: 2070), GSGSPHSLRFNQQ (SEQ ID NO: 2071), GSGSPHSYLRNQQ (SEQ ID NO: 5290), GSGSPHCSLQNQQ (SEQ ID NO: 5291), GSGSPHVLWQNQQ (SEQ ID NO: 5292), GSGSPHSKWLLQQ (SEQ ID NO: 2072), GSGSPHSLWSNQQ (SEQ ID NO: 5293), GSGSPHSKRRLQQ (SEQ ID NO: 2073), GSGSPHSVYLNQQ (SEQ ID NO: 5294), GSGSPHSLWLNQQ (SEQ ID NO: 5295), GSGSPHSKAQRKL (SEQ ID NO: 2074), GSGSPHSKALRRQ (SEQ ID NO: 2075), GSGSPHSKAQRLR (SEQ ID NO: 2076), GSGSPHSKAQNQQ (SEQ ID NO: 1801), GSGSPHSKAQRRL (SEQ ID NO: 2077), GSGSPHSKARRQQ (SEQ ID NO: 2078), GSGSPHSKARRLQ (SEQ ID NO: 2079), GSGSPHSKSRRQQ (SEQ ID NO: 2080), GSGSPHSKARLRQ (SEQ ID NO: 2082), GSGSPHSKASKRQ (SEQ ID NO: 2083), GSGSPHVRRQNQQ (SEQ ID NO: 2084), GSGSPHSKAQLYR (SEQ ID NO: 2085).

GSGSPHSLFRNQQ (SEQ ID NO: 5296). GSGSPHSKAQLTV (SEQ ID NO: 2086), or any portion thereof, e.g., any 2, 3, 4, 5, 6, 7, 8, 9, 10, 11, or 12 amino acids, e.g., consecutive amino acids, thereof. In some embodiments. [B] is present immediately subsequent to [A]. In some embodiments, the AAV capsid variant comprises (e.g., in loop IV) an amino acid sequence comprising, from N-terminus to C-terminus, [A][B].

In some embodiments, the AAV capsid variant comprises (e.g., in loop IV) an amino acid sequence having the formula [A][B](SEQ ID NO: 4699), wherein [A] comprises X1 X2 X3 X4 X5 X6 and [B] comprises SPHKSG (SEQ ID NO: 946). In some embodiments, X1 of [A] is T, M, A, C, I, R, L, D, F, V, Q, N, or H, In some embodiments. X2 of [A] is I, P, E, N, D, S, A, T, M, or Q, In some embodiments, X3 of [A] is N, E, G, Y, W, M, T, I, K, Q, F, S, V, A, or L, In some embodiments, X4 of [A] is G, D, R, or E, In some embodiments, X5 of [A] is H, Q, N, or D, In some embodiments, X6 of [A] is D or R, In some embodiments. [A] comprises TINGHD (SEQ ID NO: 5297), MPEGHD (SEQ ID NO: 5298), MEGGHD (SEQ ID NO: 5299), MEYGHD (SEQ ID NO: 5300), AEWGHD (SEQ ID NO: 5301), CEWGHD (SEQ ID NO: 5302), ANNGQD (SEQ ID NO: 5303), IPEGHD (SEQ ID NO: 5304), ADMGHD (SEQ ID NO: 5305), IEYGHD (SEQ ID NO: 5306), ADYGHD (SEQ ID NO: 5307), IETGHD (SEQ ID NO: 5308), MEWGHD (SEQ ID NO: 5309), CEYGHD (SEQ ID NO: 5310), RINGHD (SEQ ID NO: 5311), MEIGHD (SEQ ID NO: 5312), LEYGHD (SEQ ID NO: 5313), ADWGHD (SEQ ID NO: 5314), IEIGHD (SEQ ID NO: 5315), TIKDND (SEQ ID NO: 5316), DIMGHD (SEQ ID NO: 5317), FEQGHD (SEQ ID NO: 5318), MEFGHD (SEQ ID NO: 5319), CDQGHD (SEQ ID NO: 5320), LPEGHD (SEQ ID NO: 5321), IENGHD (SEQ ID NO: 5322), MESGHD (SEQ ID NO: 5323), AEIGHD (SEQ ID NO: 5324), VEYGHD (SEQ ID NO: 5325), TSNGDD (SEQ ID NO: 5326), IEVGHD (SEQ ID NO: 5327), MEMGHD (SEQ ID NO: 5328), AEVGHD (SEQ ID NO: 5329), MDAGHD (SEQ ID NO: 5330), VEWGHD (SEQ ID NO: 5331), AEQGHD (SEQ ID NO: 5332), LEWGHD (SEQ ID NO: 5333), MELGHD (SEQ ID NO: 5334), METGHD (SEQ ID NO: 5335), MEAGHD (SEQ ID NO: 5336), TINRQR (SEQ ID NO: 5337), IESGHD (SEQ ID NO: 5338), TAKDHD (SEQ ID NO: 5339), MEVGHD (SEQ ID NO: 5340), CEIGHD (SEQ ID NO: 5341), ATNGHD (SEQ ID NO: 5342), MDGGHD (SEQ ID NO: 5343), QEVGHD (SEQ ID NO: 5344), ADQGHD (SEQ ID NO: 5345), NMNGHD (SEQ ID NO: 5346), TPWEHD (SEQ ID NO: 5347), IEMGHD (SEQ ID NO: 5348), TANEHD (SEQ ID NO: 5349), QQQGHD (SEQ ID NO: 5350), TPQDHD (SEQ ID NO: 5351), HDWGHD (SEQ ID NO: 5352), IEGGHD (SEQ ID NO: 5353), or any dipeptide, tripeptide, tetmpeptide, or pentapeptide thereof. In some embodiments. [A][B] comprises TINGHDSPHKR (SEQ ID NO: 5354), MPEGHDSPHKS (SEQ ID NO: 5355), MEGGHDSPHKS (SEQ ID NO: 5356), MEYGHDSPHKS (SEQ ID NO: 5357), AEWGHDSPHKS (SEQ ID NO: 5358), CEWGHDSPHKS (SEQ ID NO: 5359), ANNGQDSPHKS (SEQ ID NO: 5360), IPEGHDSPHKS (SEQ ID NO: 5361), ADMGHDSPHKS (SEQ ID NO: 5362), IEYGHDSPHKS (SEQ ID NO: 5363), ADYGHDSPHKS (SEQ ID NO: 5364), IETGHDSPHKS (SEQ ID NO: 5365), MEWGHDSPHKS (SEQ ID NO: 5366), CEYGHDSPHKS (SEQ ID NO: 5367), RINGHDSPHKS (SEQ ID NO: 5368), MEIGHDSPHKS (SEQ ID NO: 5369), LEYGHDSPHKS (SEQ ID NO: 5370), ADWGHDSPHKS (SEQ ID NO: 5371), iEiGHDSPHKS (SEQ ID NO: 5372), TIKDNDSPHKS (SEQ ID NO: 5373), DIMGHDSPHKS (SEQ ID NO: 5374), FEQGHDSPHKS (SEQ ID NO: 5375), MEFGHDSPHKS (SEQ ID NO: 5376), CDQGHDSPHKS (SEQ ID NO: 5377), LPEGHDSPHKS (SEQ ID NO: 5378), IENGHDSPHKS (SEQ ID NO: 5379), MESGHDSPHKS (SEQ ID NO: 5380), AEIGHDSPHKS (SEQ ID NO: 5381), VEYGHDSPHKS (SEQ ID NO: 5382). TSNGDDSPHKS (SEQ ID NO: 5383), IEVGHDSPHKS (SEQ ID NO: 5384), MEMGHDSPHKS (SEQ ID NO: 5385), AEVGHDSPHKS (SEQ ID NO: 5386), MDAGHDSPHKS (SEQ ID NO: 5387), VEWGHDSPHKS (SEQ ID NO: 5388), AEQGHDSPHKS (SEQ ID NO: 5389), LEWGHDSPHKS (SEQ ID NO: 5390), MELGHDSPHKS (SEQ ID NO: 5391), METGHDSPHKS (SEQ ID NO: 5392), MEAGHDSPHKS (SEQ ID NO: 5393), TINRQRSPHKS (SEQ ID NO: 5394), IESGHDSPHKS (SEQ ID NO: 5395), TAKDHDSPHKS (SEQ ID NO: 53%), MEVGHDSPHKS (SEQ ID NO: 5397), CEIGHDSPHKS (SEQ ID NO: 5398), ATNGHDSPHKS (SEQ ID NO: 5399), MDGGHDSPHKS (SEQ ID NO: 5400), QEVGHDSPHKS (SEQ ID NO: 5401), ADQGHDSPHKS (SEQ ID NO: 5402). NMNGHDSPHKS (SEQ ID NO: 5403), TPWEHDSPHKS (SEQ ID NO: 5404), IEMGHDSPHKS (SEQ ID NO: 5405), TANEHDSPHKS (SEQ ID NO: 5406), TINGHDSPHKS (SEQ ID NO: 5407), QQQGHDSPHKS (SEQ ID NO: 5408), TPQDHDSPHKS (SEQ ID NO: 5409), HDWGHDSPHKS (SEQ ID NO: 5410), IEGGHDSPHKS (SEQ ID NO: 5411), or any portion thereof, e.g., any 2, 3, 4, 5, 6, 7, 8, 9, 10, 11, or 12 amino acids, e.g., consecutive amino acids, thereof. In some embodiments. [B] is present immediately subsequent to [A]. In some embodiments, the AAV capsid variant comprises (e.g., in loop IV) an amino acid sequence comprising, from N-terminus to C-terminus, [A][B].

In some embodiments, the AAV capsid variant comprises (e.g., in loop IV) an amino acid sequence comprising at least 3, at least 4, at least 5, at least 6, at least 7, at least 8, at least 9, at least 10, at least 11, at least 12, at least 13, at least 14, at least 15, at least 16, or at least 17 consecutive amino acids from any one of the sequences provided in Table 1, 2A, 2B, or 20-26. In some embodiments, the AAV capsid variant comprises (e.g., in loop IV) an amino acid sequence comprising at least 3, at least 4, or at least 5 consecutive amino acids from any one of SEQ ID NOs: 945-980 or 985-986. In some embodiments, the AAV capsid variant comprises (e.g., in loop TV) an amino acid sequence comprising at least 3, at least 4, at least 5, at least 6, at least 7, at least 8, at least 9, at least 10, at least 11, at least 12, or at least 13 consecutive amino acids from any one of SEQ ID NOs: 2, 200, 201, 941, 943, 204, 208, 404, or 903-909.

In some embodiments, the at least 3 consecutive amino acids comprise SPH. In some embodiments, the at least 4 consecutive amino acids comprise SPHS (SEQ ID NO: 4700). In some embodiments, the at least 5 consecutive amino acids comprise SPHSK (SEQ ID NO: 4701). In some embodiments, the at least 6 consecutive amino acids comprise SPHSKA (SEQ ID NO: 941).

In some embodiments, at least 3 consecutive amino acids comprise HDS. In some embodiments, the at least 4 consecutive amino acids comprise HDSP (SEQ ID NO: 4702). In some embodiments, the at least 5 consecutive amino acids comprise HDSPH (SEQ ID NO: 4703). In some embodiments, the at least 6 consecutive amino acids comprise HDSPHK (SEQ ID NO: 2).

In some embodiments, the at least 3 consecutive amino acids comprise SPH. In some embodiments, the at least 4 consecutive amino acids comprise SPHK (SEQ ID NO: 6398). In some embodiments, the at least 5 consecutive amino acids comprise SPHKY (SEQ ID NO: 4715). In some embodiments, the at least 6 consecutive amino acids comprise SPHKYG (SEQ ID NO: 966).

In some embodiments, the AAV capsid variant comprises (e.g., in loop IV) an amino acid sequence comprising at least one, at least two, or at least three, but no more than four modifications, relative to the amino acid sequence of any one of the sequences provided in Table 1, 2A, 2B, or 20-26. In some embodiments, the AAV capsid variant comprises (e.g., in loop IV) an amino acid sequence comprising at least one, at least two, or at least three, but no more than four different amino acids, relative to the amino acid sequence of any one of the sequences provided in Table 1, 2A, 2B, or 20-26. In some embodiments, the AAV capsid variant comprises (e.g., in loop IV) an amino acid sequence comprising at least one, at least two, or at least three, but no more than four modifications, relative to the amino acid sequence of any one of SEQ ID NOs: 945-980 or 985-986. In some embodiments, the AAV capsid variant comprises (e.g., in loop IV) an amino acid sequence comprising at least one, at least two, or at least three, but no more than four different amino acids, relative to the amino acid sequence of any one of SEQ ID NOs: 945-980 or 985-986. In some embodiments, the AAV capsid variant comprises (e.g., in loop IV) an amino acid sequence comprising at least one, at least two, or at least three, but no more than four modifications, relative to the amino acid sequence of any one of SEQ ID NOs: 2, 200, 201, 941, 943, 204, 208, 404, or 903-909. In some embodiments, the AAV capsid variant comprises (e.g., in loop IV) an amino acid sequence comprising at least one, at least two, or at least three, but no more than four different amino acids relative to the amino acid sequence of any one of SEQ ID NOs: 2, 200, 201, 941, 943, 204, 208, 404, or 903-909. In some embodiments, the AAV capsid variant comprises (e.g., in loop IV) an amino acid sequence comprising at least one, at least two, or at least three, but no more than four modifications, relative to the amino acid sequence of SEQ ID NO: 3589. In some embodiments, the AAV capsid variant comprises (e.g., in loop IV) an amino acid sequence comprising at least one, at least two, or at least three, but no more than four different amino acids relative to the amino acid sequence of SEQ ID NO: 3589. In some embodiments, the AAV capsid variant comprises (e.g., in loop IV) an amino acid sequence comprising at least one, at least two, or at least three, but no more than four modifications, relative to the amino acid sequence of SEQ ID NO: 1754. In some embodiments, the AAV capsid variant comprises (e.g., in loop IV) an amino acid sequence comprising at least one, at least two, or at least three, but no more than four different amino acids relative to the amino acid sequence of SEQ ID NO: 1754.

In some embodiments, the AAV capsid variant comprises (e.g., in loop IV) an amino acid sequence comprising at least one, at least two, or at least three, but no more than four modifications, relative to the amino acid sequence of SPHSKA (SEQ ID NO: 941). In some embodiments, the peptide comprises an amino acid sequence comprising at least one, at least two, or at least three, but no more than four different amino acids relative to the amino acid sequence of SPHSKA (SEQ ID NO: 941).

In some embodiments, the AAV capsid variant comprises (e.g., in loop IV) an amino acid sequence comprising at least one, at least two, or at least three, but no more than four modifications, relative to the amino acid sequence of HDSPHK (SEQ ID NO: 2). In some embodiments, the AAV capsid variant comprises (e.g., in loop IV) an amino acid sequence comprising at least one, at least two, or at least three, but no more than four different amino acids relative to the amino acid sequence of HDSPHK (SEQ ID NO: 2).

In some embodiments, the AAV capsid variant comprises (e.g., in loop IV) an amino acid sequence comprising at least one, at least two, or at least three, but no more than four modifications, relative to the amino acid sequence of SPHKYG (SEQ ID NO: 96). In some embodiments, the AAV capsid variant comprises (e.g., in loop IV) an amino acid sequence comprising at least one, at least two, or at least three, but no more than four different amino acids relative to the amino acid sequence of SPHKYG (SEQ ID NO: 966).

In some embodiments, the AAV capsid variant comprises (e.g., in loop IV) the amino acid sequence of any of the sequences provided in Table 1, 2A, 2B, or 20-26. In some embodiments, the AAV capsid variant comprises (e.g., in loop IV) the amino acid sequence of any of SEQ ID NOs: 945-980 or 985-986. In some embodiments, the AAV capsid variant comprises (e.g., in loop IV) the amino acid sequence of any of SEQ ID NOs: 2, 200, 201, 941, 943, 204, 208, 404, or 903-909. In some embodiments, the AAV capsid variant comprises (e.g., in loop IV) the amino acid sequence of SEQ ID NO: 941. In some embodiments, the AAV capsid variant comprises (e.g., in loop IV) the amino acid sequence of SEQ ID NO: 943. In some embodiments, the AAV capsid variant comprises (e.g., in loop IV) the amino acid sequence of SEQ ID NO: 2. In some embodiments, the AAV capsid variant comprises (e.g., in loop IV) the amino acid sequence of SEQ ID NO: 3589. In some embodiments, the AAV capsid variant comprises (e.g., in loop IV) the amino acid sequence of SEQ ID NO: 1754. In some embodiments, the AAV capsid variant comprises (e.g., in loop IV) the amino acid sequence of SEQ ID NO: 3241. In some embodiments, the AAV capsid variant comprises (e.g., in loop IV) the amino acid sequence of SEQ ID NO: 4100. In some embodiments, the AAV capsid variant comprises (e.g., in loop IV) the amino acid sequence of SEQ ID NO: 4062. In some embodiments, the AAV capsid variant comprises (e.g., in loop IV) the amino acid sequence of SEQ ID NO: 4486.

In some embodiments, the AAV capsid variant comprises (e.g., in loop IV) an amino acid sequence encoded by a nucleotide sequence described herein, e.g., a nucleotide sequence of Table 2A. In some embodiments, the AAV capsid variant comprises (e.g., in loop IV) an amino acid sequence encoded by a nucleotide sequence comprising at least one, at least two, at least three, at least four, at least five, at least six, or at least seven modifications, but no more than ten modifications, relative to the nucleotide sequence of SEQ ID NO: 942. In some embodiments, the AAV capsid variant comprises (e.g., in loop IV) an amino acid sequence encoded by a nucleotide sequence comprising at least one, at least two, at least three, at least four, at least five, at least six, or at least seven, but no more than ten different nucleotides, relative to the nucleotide sequence of SEQ ID NO: 942. In some embodiments, the AAV capsid variant comprises (e.g., in loop IV) an amino acid sequence encoded by the nucleotide sequence of SEQ ID NO: 942, or a nucleotide sequence substantially identical (e.g., having at least 70%, at least 75%, at least 80%, at least 85%, at least 90%, at least 92% o, at least 95%, at least 97%, at least 98%, or at least 99% sequence identity) thereto. In some embodiments, the AAV capsid variant comprises (e.g., in loop IV) an amino acid sequence encoded by a nucleotide sequence comprising at least one, at least two, at least three, at least four, at least five, at least six, or at least seven modifications, but no more than ten modifications, relative to the nucleotide sequence of SEQ ID NO: 944. In some embodiments, the AAV capsid variant comprises (e.g., in loop IV) an amino acid sequence encoded by a nucleotide sequence comprising at least one, at least two, at least three, at least four, at least five, at least six, or at least seven, but no more than ten different nucleotides, relative to the nucleotide sequence of SEQ ID NO: 944. In some embodiments, the AAV capsid variant comprises (e.g., in loop IV) an amino acid sequence encoded by the nucleotide sequence of SEQ ID NO: 944, or a nucleotide sequence substantially identical (e.g., having at least 70%, at least 75%, at least 80%, at least 85%, at least 90%, at least 92%, at least 95%, at least 97%, at least 98%, or at least 99% sequence identity) thereto.

In some embodiments, the AAV capsid variant comprises (e.g., in loop IV) an amino acid sequence encoded by a nucleotide sequence described herein, e.g., a nucleotide sequence of Table 2A. In some embodiments, the nucleotide sequence is codon optimized. In some embodiments, the nucleotide sequence is an isolated nucleotide sequence. In some embodiments, the nucleotide sequence is a recombinant nucleotide sequence.

In some embodiments, the nucleotide sequence encoding an AAV capsid variant comprises the nucleotide sequence of SEQ ID NO: 942, or a nucleotide sequence comprising at least one, at least two, at least three, at least four, at least five, at least six, or at least seven modifications, but no more than ten modifications, relative to the nucleotide sequence of SEQ ID NO: 942. In some embodiments, the nucleotide sequence encoding an AAV capsid variant comprises a nucleotide sequence comprising at least one, at least two, at least three, at least four, at least five, at least six, or at least seven, but no more than ten different nucleotides, relative to the nucleotide sequence of SEQ ID NO: 942. In some embodiments the nucleic acid sequence encoding an AAV capsid variant comprises a nucleotide sequence comprising the nucleotide sequence of SEQ ID NO: 942, or a nucleotide sequence substantially identical (e.g., having at least 70%, at least 75%, at least 80%, at least 85%, at least 90%, at least 92%, at least 95%, at least 97%, at least 98%, or at least 99% sequence identity) thereto.

In some embodiments, the nucleic acid encoding an AAV capsid variant comprises the nucleotide sequence of SEQ ID NO: 3, or a nucleotide sequence comprising at least one, at least two, at least three, at least four, at least five, at least six, or at least seven modifications, but no more than ten modifications, relative to the nucleotide sequence of SEQ ID NO: 3. In some embodiments, the nucleotide sequence encoding an AAV capsid variant comprises a nucleotide sequence comprising at least one, at least two, at least three, at least four, at least five, at least six, or at least seven, but no more than ten different nucleotides, relative to the nucleotide sequence of SEQ ID NO: 3. In some embodiments, the nucleic acid encoding an AAV capsid variant comprises a nucleotide sequence comprising the nucleotide sequence of SEQ ID NO: 3, or a nucleotide sequence substantially identical (e.g., having at least 70%, at least 75%, at least 80%, at least 85%, at least 90%, at least 92%, at least 95%, at least 97%, at least 98%, or at least 99% sequence identity) thereto.

In some embodiments, the nucleic acid encoding an AAV capsid variant comprises the nucleotide sequence of SEQ ID NO: 944, or a nucleotide sequence comprising at least one, at least two, at least three, at least four, at least five, at least six, or at least seven modifications, but no more than ten modifications, relative to the nucleotide sequence of SEQ ID NO: 944. In some embodiments, the nucleotide sequence encoding an AAV capsid variant comprises a nucleotide sequence comprising at least one, at least two, at least three, at least four, at least five, at least six, or at least seven, but no more than ten different nucleotides, relative to the nucleotide sequence of SEQ ID NO: 944. In some embodiments the nucleic acid encoding an AAV capsid variant comprises a nucleotide sequence comprising the nucleotide sequence of SEQ ID NO: 944, or a nucleotide sequence substantially identical (e.g., having at least 70%, at least 75%, at least 80%, at least 85%, at least 90%, at least 92%, at least 95%, at least 97%, at least 98%, or at least 99% sequence identity) thereto.

Also provided herein are polynucleotide sequences encoding any of the AAV capsid variants described above, AAV particles, vectors, and cells comprising the same.

In some embodiments. [N1]-[N2]-[N3] is present in loop IV of the AAV capsid variant. In some embodiments [N0] and [N4] are present in loop IV of the AAV capsid variant. In some embodiments. [N1]-[N1]-[N2]-[N3]-[N4] is present in loop IV of the AAV capsid variant.

In some embodiments, [N0] is present immediately subsequent to amino acid 449, relative to a reference sequence of SEQ ID NO: 138 (i.e., at a sequence position corresponding to that in SEQ ID NO: 138). In some embodiments. [N0] is present immediately subsequent to amino acid 449, numbered according to SEQ ID NO: 4, 36, 981, or 982 (i.e., at a sequence position corresponding to that in SEQ ID NO: 4, 36, 981, or 982). In some embodiments, [N0] replaces amino acids 450, 451, and 452 (e.g., amino acids T450, 1451, and N452), numbered according to SEQ ID NO: 4, 36, 138, 981, or 982. In some embodiments, [N0] is present immediately subsequent to amino acid 449 and [N0] replaces amino acids 450-452 (e.g., T450, 1451, and N452), numbered according to SEQ ID NO: 4, 36, 138, 981, or 982. In some embodiments. [N1] is present immediately subsequent to amino acid 452, numbered according to SEQ ID NO: 4, 36, 138, 981, or 982. In some embodiments. [N1] replaces amino acids 453-455 (e.g., G453, S454, and G455), numbered according to SEQ ID NO: 4, 36, 138, 981, or 982. In some embodiments, [N1] is present immediately subsequent to amino acid 452 and wherein [N1] replaces amino acids 453-455 (e.g., G453, S454, and G455), numbered according to SEQ ID NO: 4, 36, 138, 981, or 982. In some embodiments, [N2] is present immediately subsequent to amino acid 455, numbered according to SEQ ID NO: 4, 36, 138, 981, or 982. In some embodiments, [N2]-[N3] is present immediately subsequent to amino acid 455, numbered according to SEQ ID NO: 4, 36, 138, 981, or 982. In some embodiments [N1]-[N2]-[N3] is present immediately subsequent to amino acid 452, numbered according to SEQ ID NO: 4, 36, 138, 981, or 982. In some embodiments, [N1]-[N2]-[N3] replaces amino acids 453-455 (e.g., G453, S454, and G455), numbered according to SEQ ID NO: 4, 36, 138, 981, or 982. In some embodiments, [N1] is present immediately subsequent to amino acid 452 and wherein [N1]-[N2]-[N3] replaces amino acids 453-455 (e.g., G453, S454, and G455), numbered according to SEQ ID NO: 4, 36, 138, 981, or 982. In some embodiments, [N4] is present immediately subsequent to amino acid 455, numbered according to SEQ ID NO: 138. In some embodiments. [N4] replaces amino acids 456-459 (e.g., Q456, N457, Q458, and Q459), numbered according to SEQ ID NO: 138. In some embodiments, [N4] is present immediately subsequent to amino acid 455, and [N4] replaces amino acids 456459 (e.g., Q456, N457, Q458, and Q459), numbered according to SEQ ID NO: 138. In some embodiments, [N2]-[N3]-[N4] replaces amino acids 456459 (e.g., Q456, N457, Q458, and Q459), numbered according to SEQ ID NO: 138. In some embodiments, [N2]-[N3]-[N4] is present immediately subsequent to amino acid 455, wherein [N2]-[N3]-[N4] replaces amino acids 456459 (e.g., Q456, N457, Q458, and Q459), numbered according to SEQ ID NO: 138. In some embodiments, [N1]-[N2]-[N3]-[N4] replaces amino acids 453-459 (e.g., G453, S454, G455, Q456, N457, Q458, and Q459), numbered according to SEQ ID NO: 138. In some embodiments, [N1]-[N2]-[N3]-[N4] is present immediately subsequent to amino acid 452, and [N1]-[N2]-[N3]-[N4] replaces amino acids 453459 (e.g., G453, 5454, G455. Q456, N457, Q458, and Q459), numbered according to SEQ ID NO: 138. In some embodiments, [N0]-[N11-[N2]-[N3]-[N4] replaces amino acids 450459 (e.g., T450, 1451, N452, G453, S454, G455, Q456, N457, Q458, and Q459), numbered according to SEQ ID NO: 138. In some embodiments. [N0]-[N1]-[N2]-[N3]-[N4] is present immediately subsequent to amino acid 449, and wherein [N0]-[N1]-[N2]-[N3]-[N4] replaces amino acids 450459 (e.g., T450, 1451, N452, G453, 5454, G455, Q456, N457, Q458, and Q459), numbered according to SEQ ID NO: 138.

In some embodiments, [N3] is present immediately subsequent to [N2].

In some embodiments, the AAV capsid variant comprises, from N-terminus to C-terminus, [N2]-[N3]. In some embodiments, the AAV capsid variant comprises, from N-terminus to C-terminus, [N1]-[N2]-[N31. In some embodiments, the AAV capsid variant comprises, from N-terminus to C-terminus. [N1]-[N2]-[N3]-[N4]. In some embodiments, the AAV capsid variant comprises, from N-terminus to C-terminus, [N0]-[N1]-[N2]-[N3]. In some embodiments, the AAV capsid variant comprises, from N-terminus to C-terminus, [N0]-[N1]-[N2]-[N3]-[N4].

In some embodiments, an AAV capsid variant comprises an amino acid sequence having the formula [A][B] (SEQ ID NO: 4694), wherein [A] comprises the amino acid sequence of GSGSPH (SEQ ID NO: 4695) and [B] comprises X1 X2 X3 X4 X5 X6 X7. In some embodiments, X1 of [B] is S, C, F, or V, In some embodiments. X2 of [B] is K, L, R, I, E, Y, V, or S, In some embodiments. X3 of [B] is A, R, L, G, I, Y, S, F, or W, In some embodiments. X4 of [B] is W, Q, R, G, L, V, S, or F. In some embodiments. X5 of [B] is N, Y, R, C, K, or L, In some embodiments, X6 of [B] is Q, G, K, R, T, L, or Y, In some embodiments, X7 of [B] is Q, L, R, or V, In some embodiments. [B] comprises SLLWNQQ (SEQ ID NO: 5247), SKAQYYV (SEQ ID NO: 5248), SKLRRQQ (SEQ ID NO: 5249), SIWQNQQ (SEQ ID NO: 5250), SKAGCGQ (SEQ ID NO: 5251), SRAQNQQ (SEQ ID NO: 5252), SKRLRQQ (SEQ ID NO: 5253), SLRRNQQ (SEQ ID NO: 5254), SRGRNQQ (SEQ ID NO: 5255), SEIVNQQ (SEQ ID NO: 5256), SSRRNQQ (SEQ ID NO: 5257), CLLQNQQ (SEQ ID NO: 5258), SKAFRLQ (SEQ ID NO: 5259), CLAQNQQ (SEQ ID NO: 5260), FLRQNQQ (SEQ ID NO: 5261), SLRFNQQ (SEQ ID NO: 5262), SYLRNQQ (SEQ ID NO: 5263), CSLQNQQ (SEQ ID NO: 5264), VLWQNQQ (SEQ ID NO: 5265), SKWLLQQ (SEQ ID NO: 5266), SLWSNQQ (SEQ ID NO: 5267), SKRRLQQ (SEQ ID NO: 5268), SVYLNQQ (SEQ ID NO: 5269), SLWLNQQ (SEQ ID NO: 5270), SKAQRKL (SEQ ID NO: 5271), SKALRRQ (SEQ ID NO: 5272), SKAQRLR (SEQ ID NO: 5273), SKAQNQQ (SEQ ID NO: 5274), SKAQRRL (SEQ ID NO: 5275), SKARRQQ (SEQ ID NO: 5276), SKARRLQ (SEQ ID NO: 5277), SKSRRQQ (SEQ ID NO: 5278), SKARLRQ (SEQ ID NO: 5279), SKASKRQ (SEQ ID NO: 5280), VRRQNQQ (SEQ ID NO: 5281), SKAQLYR (SEQ ID NO: 5282), SLFRNQQ (SEQ ID NO: 5283), SKAQLTV (SEQ ID NO: 5284), or any dipeptide, tinpeptide, tetrapeptide, pentapeptide, or hexapeptide thereof. In some embodiments, [A][B] comprises GSGSPHSLLWNQQ (SEQ ID NO: 5285), GSGSPHSKAQYYV (SEQ ID NO: 2060), GSGSPHSKLRRQQ (SEQ ID NO: 2061), GSGSPHSIWQNQQ (SEQ ID NO: 5286), GSGSPHSKAGCGQ (SEQ ID NO: 2062), GSGSPHSRAQNQQ (SEQ ID NO: 2063), GSGSPHSKRLRQQ (SEQ ID NO: 2064), GSGSPHSLRRNQQ (SEQ ID NO: 2065), GSGSPHSRGRNQQ (SEQ ID NO: 2066), GSGSPHSEIVNQQ (SEQ ID NO: 5287), GSGSPHSSRRNQQ (SEQ ID NO: 2067), GSGSPHCLLQNQQ (SEQ ID NO: 5288), GSGSPHSKAFRLQ (SEQ ID NO: 2068), GSGSPHCLAQNQQ (SEQ ID NO: 5289), GSGSPHFLRQNQQ (SEQ ID NO: 2070), GSGSPHSLRFNQQ (SEQ ID NO: 2071), GSGSPHSYLRNQQ (SEQ ID NO: 5290), GSGSPHCSLQNQQ (SEQ ID NO: 5291), GSGSPHVLWQNQQ (SEQ ID NO: 5292), GSGSPHSKWLLQQ (SEQ ID NO: 2072), GSGSPHSLWSNQQ (SEQ ID NO: 5293), GSGSPHSKRRLQQ (SEQ ID NO: 2073), GSGSPHSVYLNQQ (SEQ ID NO: 5294), GSGSPHSLWLNQQ (SEQ ID NO: 5295), GSGSPHSKAQRKL (SEQ ID NO: 2074), GSGSPHSKALRRQ (SEQ ID NO: 2075), GSGSPHSKAQRLR (SEQ ID NO: 2076), GSGSPHSKAQNQQ (SEQ ID NO: 1801), GSGSPHSKAQRRL (SEQ ID NO: 2077), GSGSPHSKARRQQ (SEQ ID NO: 2078), GSGSPHSKARRLQ (SEQ ID NO: 2079), GSGSPHSKSRRQQ (SEQ ID NO: 2080), GSGSPHSKARLRQ (SEQ ID NO: 2082),GSGSPHSKASKRQ (SEQ ID NO: 2083), GSGSPHVRRQNQQ (SEQ ID NO: 2084), GSGSPHSKAQLYR (SEQ ID NO: 2085), GSGSPHSLFRNQQ (SEQ ID NO: 5296), GSGSPHSKAQLTV (SEQ ID NO: 2086), or any portion thereof, e.g., any 2, 3, 4, 5, 6, 7, 8, 9, 10, 11, or 12 amino acids, e.g., consecutive amino acids, thereof.

In some embodiments, [A][B] is present in loop IV of the AAV capsid variant. In some embodiments, [A] is present immediately subsequent to amino acid 452, relative to a reference sequence of SEQ ID NO: 138 (i.e., at a sequence position corresponding to that in SEQ ID NO: 138). In some embodiments, [A] replaces amino acids 453-455 (e.g., G453, S454, G455), relative to a reference sequence of SEQ ID NO: 138. In some embodiments, [A] is present immediately subsequent to amino acid 452, and [A] replaces amino acids 453-455 (e.g., G453, S454, G455), relative to a reference sequence of SEQ ID NO: 138. In some embodiments. [B] is present immediately subsequent to [A]. In some embodiments, [B] replaces amino acids 456-459 (e.g., Q456, N457, Q458, Q459), relative to a reference sequence of SEQ ID NO: 138. In some embodiments, [A][B] replaces amino acids 453-459 (e.g., G453, S454, G455, Q456, N457, Q458, Q459), relative to a reference sequence of SEQ ID NO: 138. In some embodiments, [A][B] is present immediately subsequent to amino acid 452, and wherein [A][B] replaces amino acids 453459 (e.g., G453, S454, G455, Q456, N457, Q458, Q459), relative to a reference sequence of SEQ ID NO: 138. In some embodiments, the AAV capsid variant comprises, from N-terminus to C-terminus, [A][B].

In some embodiments, an AAV capsid variant comprises an amino acid sequence having the formula [A][B](SEQ ID NO: 4699), wherein [A] comprises X1 X2 X3 X4 X5 X6 and [B] comprises SPHKSG (SEQ ID NO: 946). In some embodiments, X1 of [AJ is T, M, A, C, I, R, L, D, F, V, Q, N, or H, In some embodiments, X2 of [A] is I, P, E, N, D, S, A, T, M, or Q, In some embodiments. X3 of [A] is N, E, G, Y, W, M, T, 1, K, Q, F, S, V, A, or L, In some embodiments, X4 of [A] is G, D, R, or E, In some embodiments. X5 of [A] is H, Q, N, or D, In some embodiments, X6 of [A] is D or R, In some embodiments, [A] comprises TINGHD (SEQ ID NO: 5297), MPEGHD (SEQ ID NO: 5298), MEGGHD (SEQ ID NO: 5299), MEYGHD (SEQ ID NO: 5300), AEWGHD (SEQ ID NO: 5301), CEWGHD (SEQ ID NO: 5302), ANNGQD (SEQ ID NO: 5303), IPEGHD (SEQ ID NO: 5304), ADMGHD (SEQ ID NO: 5305), IEYGHD (SEQ ID NO: 5306), ADYGHD (SEQ ID NO: 5307), IETGHD (SEQ ID NO: 5308), MEWGHD (SEQ ID NO: 5309), CEYGHD (SEQ ID NO: 5310), RINGHD (SEQ ID NO: 5311), MEIGHD (SEQ ID NO: 5312), LEYGHD (SEQ ID NO: 5313), ADWGHD (SEQ ID NO: 5314), IEIGHD (SEQ ID NO: 5315). TIKDND (SEQ ID NO: 5316), DIMGHD (SEQ ID NO: 5317), FEQGHD (SEQ ID NO: 5318), MEFGHD (SEQ ID NO: 5319), CDQGHD (SEQ ID NO: 5320), LPEGHD (SEQ ID NO: 5321), IENGHD (SEQ ID NO: 5322), MESGHD (SEQ ID NO: 5323), AEIGHD (SEQ ID NO: 5324), VEYGHD (SEQ ID NO: 5325), TSNGDD (SEQ ID NO: 5326), IEVGHD (SEQ ID NO: 5327), MEMGHD (SEQ ID NO: 5328), AEVGHD (SEQ ID NO: 5329), MDAGHD (SEQ ID NO: 5330), VEWGHD (SEQ ID NO: 5331), AEQGHD (SEQ ID NO: 5332), LEWGHD (SEQ ID NO: 5333), MELGHD (SEQ ID NO: 5334), METGHD (SEQ ID NO: 5335), MEAGHD (SEQ ID NO: 5336). TINRQR (SEQ ID NO: 5337), IESGHD (SEQ ID NO: 5338), TAKDHD (SEQ ID NO: 5339), MEVGHD (SEQ ID NO: 5340). CEIGHD (SEQ ID NO: 5341), ATNGHD (SEQ ID NO: 5342), MDGGHD (SEQ ID NO: 5343), QEVGHD (SEQ ID NO: 5344), ADQGHD (SEQ ID NO: 5345), NMNGHD (SEQ ID NO: 5346), TPWEHD (SEQ ID NO: 5347), IEMGHD (SEQ ID NO: 5348), TANEHD (SEQ ID NO: 5349), QQQGHD (SEQ ID NO: 5350), TPQDHD (SEQ ID NO: 5351), HDWGHD (SEQ ID NO: 5352), IEGGHD (SEQ ID NO: 5353), or any dipeptide, tripeptide, tetrapeptide, orpentapeptide thereof. In some embodiments, [A][B] comprises TINGHDSPHKR (SEQ ID NO: 5354), MPEGHDSPHKS (SEQ ID NO: 5355), MEGGHDSPHKS (SEQ ID NO: 5356), MEYGHDSPHKS (SEQ ID NO: 5357), AEWGHDSPHKS (SEQ ID NO: 5358), CEWGHDSPHKS (SEQ ID NO: 5359), ANNGQDSPHKS (SEQ ID NO: 5360). IPEGHDSPHKS (SEQ ID NO: 5361), ADMGHDSPHKS (SEQ ID NO: 5362), IEYGHDSPHKS (SEQ ID NO: 5363), ADYGHDSPHKS (SEQ ID NO: 5364), IETGHDSPHKS (SEQ ID NO: 5365), MEWGHDSPHKS (SEQ ID NO: 5366), CEYGHDSPHKS (SEQ ID NO: 5367), RINGHDSPHKS (SEQ ID NO: 5368), MEIGHDSPHKS (SEQ ID NO: 5369), LEYGHDSPHKS (SEQ ID NO: 5370), ADWGHDSPHKS (SEQ ID NO: 5371), IEIGHDSPHKS (SEQ ID NO: 5372), TIKDNDSPHKS (SEQ ID NO: 5373). DIMGHDSPHKS (SEQ ID NO: 5374). FEQGHDSPHKS (SEQ ID NO: 5375), MEFGHDSPHKS (SEQ ID NO: 5376), CDQGHDSPHKS (SEQ ID NO: 5377), LPEGHDSPHKS (SEQ ID NO: 5378), IENGHDSPHKS (SEQ ID NO: 5379), MESGHDSPHKS (SEQ ID NO: 5380), AEIGHDSPHKS (SEQ ID NO: 5381), VEYGHDSPHKS (SEQ ID NO: 5382). TSNGDDSPHKS (SEQ ID NO: 5383), IEVGHDSPHKS (SEQ ID NO: 5384), MEMGHDSPHKS (SEQ ID NO: 5385), AEVGHDSPHKS (SEQ ID NO: 5386), MDAGHDSPHKS (SEQ ID NO: 5387), VEWGHDSPHKS (SEQ ID NO: 5388), AEQGHDSPHKS (SEQ ID NO: 5389), LEWGHDSPHKS (SEQ ID NO: 5390), MELGHDSPHKS (SEQ ID NO: 5391), METGHDSPHKS (SEQ ID NO: 5392), MEAGHDSPHKS (SEQ ID NO: 5393), TINRQRSPHKS (SEQ ID NO: 5394), IESGHDSPHKS (SEQ ID NO: 5395). TAKDHDSPHKS (SEQ ID NO: 5396), MEVGHDSPHKS (SEQ ID NO: 5397). CEIGHDSPHKS (SEQ ID NO: 5398), ATNGHDSPHKS (SEQ ID NO: 5399), MDGGHDSPHKS (SEQ ID NO: 5400), QEVGHDSPHKS (SEQ ID NO: 5401). ADQGHDSPHKS (SEQ ID NO: 5402), NMNGHDSPHKS (SEQ ID NO: 5403), TPWEHDSPHKS (SEQ ID NO: 5404), IEMGHDSPHKS (SEQ ID NO: 5405). TANEHDSPHKS (SEQ ID NO: 5406). TINGHDSPHKS (SEQ ID NO: 5407), QQQGHDSPHKS (SEQ ID NO: 5408), TPQDHDSPHKS (SEQ ID NO: 5409), HDWGHDSPHKS (SEQ ID NO: 5410), IEGGHDSPHKS (SEQ ID NO: 5411), or any portion thereof, e.g., any 2, 3, 4, 5, 6, 7, 8, 9, 10, 11, or 12 amino acids, e.g., consecutive amino acids, thereof.

In some embodiments, [A][B] is present in loop IV of the AAV capsid variant. In some embodiments, [A] is preseit immediately subsequent to amino acid 449, relative to a reference sequence of SEQ ID NO: 138. In some embodiments, [A] replaces amino acids 450455 (e.g., T450, 1451, N452, G453, S454, G455), relative to a reference sequence of SEQ ID NO: 138. In some embodiments. [A] is present immediately subsequent to amino acid 449, and wherein [A] replaces amino acids 450-455 (e.g., T450, 1451, N452, G453, 5454, G455), relative to a reference sequence of SEQ ID NO: 138. In some embodiments, [B] is present immediately subsequent to [A]. In some embodiments. [A][B] replaces amino acids 450455 (e.g., T450, 1451, N452, G453, S454, G455), relative to a reference sequence of SEQ ID NO: 138. In some embodiments, [A][B] is present immediately subsequent to amino acid 449, and wherein [A][B] replaces amino acids 450-455 (e.g., T450, 1451, N452, G453, S454, G455), relative to a reference sequence of SEQ ID NO: 138. In some embodiments, the peptide comprises, from N-terminus to C-terminus. [AI [BJ.

In some embodiments, an AAV capsid variant comprises an amino acid sequence comprising at least 3, at least 4, at least 5, at least 6, at least 7, at least 8, at least 9, at least 10, at least 11, at least 12, at least 13, at least 16, or at least 17 consecutive amino acids from any one of the sequences provided in Table 1, 2A, 2B, or 20-26. In some embodiments, the AAV capsid variant comprises an amino acid sequence comprising at least 3, at least 4, or at least 5 consecutive amino acids from any one of SEQ ID NOs: 945-980 or 985-986. In some embodiments, the AAV capsid variant comprises an amino acid sequence comprising at least 3, at least 4, at least 5, at least 6, at least 7, at least 8, at least 9, at least 10, at least 11, at least 12, or at least 13 consecutive amino acids from any one of SEQ ID NOs: 2, 200, 201, 941, 943, 204, 208, 404, or 903-909. In some embodiments, the amino acid sequence is present in loop IV. In some embodiments, the amino acid sequence is present immediately subsequent to amino acid 448, 452, 453, or 455, numbered according to SEQ ID NO: 4, 36, 138, 981, or 982 (i.e., at a sequence position corresponding to that in SEQ ID NO: 4, 36, 138, 981, or 982). In some embodiments, the amino acid sequence is present immediately subsequent to amino acid 455, numbered according to SEQ ID NO: 982. In some embodiments, the amino acid sequence is present immediately subsequent to amino acid 455, numbered according to SEQ ID NO: 138. In some embodiments, the amino acid sequence is present immediately subsequent to amino acid 453, numbered according to SEQ ID NO: 981. In some embodiments, the amino acid sequence is present immediately subsequent to amino acid 453, numbered according to SEQ ID NO: 138. In some embodiments, the amino acid sequence replaces 1, 2, 3, 4, 5, 6, 7, 8, 9, 10, 11, or all of amino acids 499 (e.g., K499), 450 (e.g., T450). 451 (e.g., I451). 452 (e.g., N452). 453 (e.g., G453), 454 (e.g., S454), 455 (e.g., G455), 456 (e.g., Q456). 457 (e.g., N457). 458 (e.g., Q458). 459 (e.g., Q459), and 460 (e.g., T460), numbered according to SEQ ID NO: 138. In some embodiments, the AAV capsid variant comprises one or more amino acid substitutions at amino acids 499 (e.g., K499), 450 (e.g., T450). 451 (e.g., I451). 452 (e.g., N452). 453 (e.g., G453). 454 (e.g., S454), 455 (e.g., G455), 456 (e.g., Q456), 457 (e.g., N457), 458 (e.g., Q458), 459 (e.g., Q459), and/or 460 (e.g., T460), numbered according to SEQ ID NO: 138.

In some embodiments, the at least 3 consecutive amino acids comprise SPH. In some embodiments, the at least 3 consecutive amino acids comprise SPH in an AAV9 variant. In some embodiments, the at least 4 consecutive amino acids comprise SPHS (SEQ ID NO: 4700). In some embodiments, the at least 4 consecutive amino acids comprise SPHS (SEQ ID NO: 4700) in an AAV9 variant. In some embodiments, the at least 5 consecutive amino acids comprise SPHSK (SEQ ID NO: 4701). In some embodiments, the at least 5 consecutive amino acids comprise SPHSK (SEQ ID NO: 4701) in an AAV9 variant. In some embodiments, the at least 6 consecutive amino acids comprise SPHSKA (SEQ ID NO: 941). In some embodiments, the at least 6 consecutive amino acids comprise SPHSKA (SEQ ID NO: 941) in an AAV9 variant.

In some embodiments, the amino acid sequence of SPHSKA (SEQ ID NO: 941) is present at amino acids 456-461, numbered according to SEQ ID NO: 981. In some embodiments, the amino acid sequence of SPHSKA (SEQ ID NO: 941) is present at amino acids 456-461 of an AAV9 variant, numbered according to SEQ ID NO: 981. In some embodiments, the amino acid sequence of SPHSKA (SEQ ID NO: 941) is present at amino acids 456-461, numbered according to SEQ ID NO: 4. In some embodiments, the amino acid sequence of SPHSKA (SEQ ID NO: 941) is present at amino acids 456461 of an AAV9 variant, numbered according to SEQ ID NO: 4. In some embodiments, the amino acid sequence of SPHSKA (SEQ ID NO: 941) is present at amino acids 456-461, numbered according to SEQ ID NO: 36. In some embodiments, the amino acid sequence of SPHSKA (SEQ ID NO: 941) is present at amino acids 456-461 of an AAV9 variant, numbered according to SEQ ID NO: 36.

In some embodiments, the at least 3 consecutive amino acids comprise HDS. In some embodiments, the at least 3 consecutive amino acids comprise HDS in an AAV9 variant. In some embodiments, the at least 4 consecutive amino acids comprise HDSP (SEQ ID NO: 4702). In some embodiments, the at least 4 consecutive amino acids comprise HDSP (SEQ ID NO: 4702) in an AAV9 variant. In some embodiments, the at least 5 consecutive amino acids comprise HDSPH (SEQ ID NO: 4703). In some embodiments, the at least 5 consecutive amino acids comprise HDSPH (SEQ ID NO: 4703) in an AAV9 variant. In some embodiments, the at least 6 consecutive amino acids comprise HDSPHK (SEQ ID NO: 2). In some embodiments, the at least 6 consecutive amino acids comprise HDSPHK (SEQ ID NO: 2) in an AAV9 variant.

In some embodiments, the amino acid sequence of HDSPHK (SEQ ID NO: 2) is present in at amino acids 454459, numbered according to SEQ ID NO: 982. In some embodiments, the amino acid sequence of HDSPHK (SEQ ID NO: 2) is present in an AAV9 variant at amino acids 454459, numbered according to SEQ ID NO: 982.

In some embodiments, an AAV capsid variant comprises an amino acid sequence comprising at least one, at least two, or at least three, but no more than four modifications, relative to the amino acid sequence of any one of the sequences provided in Table 1, 2A, 2B, or 20-26. In some embodiments, the AAV capsid variant comprises an amino acid sequence comprising at least one, at least two, or at least three, but no more than four different amino acids, relative to the amino acid sequence of any one of the sequences provided in Table 1, 2A, 2B, or 20-26. In some embodiments, the AAV capsid variant comprises an amino acid sequence comprising at least one, at least two, or at least three, but no more than four modifications, relative to the amino acid sequence of any one of SEQ ID NOs: 945-980 or 985-986. In some embodiments, the AAV capsid variant comprises an amino acid sequence comprising at least one, at least two, or at least three, but no more than four different amino acids, relative to the amino acid sequence of any one of SEQ ID NOs: 945-980 or 985-986. In sonic embodiments, the AAV capsid variant comprises an amino acid sequence comprising at least one, at least two, or at least three, but no more than four modifications, relative to the amino acid sequence of any one of SEQ ID NOs: 2, 200, 201, 941, 943, 204, 208, 404, or 903-909. In some embodiments, the AAV capsid variant comprises an amino acid sequence comprising at least one, at least two, or at least three, but no more than four different amino acids, from the amino acid sequence of any one of SEQ ID NOs: 2, 200, 201, 941, 943, 204, 208, 404, or 903-909. In some embodiments, the amino acid sequence is present in loop IV. In some embodiments, the amino acid sequence is present immediately subsequent to amino acid 448, 452, 453, or 455, numbered according to SEQ ID NO: 4, 36, 138, 981, or 982. In some embodiments, the amino acid sequence is present immediately subsequent to amino acid 455, numbered according to SEQ ID NO: 982. In some embodiments, the amino acid sequence is present immediately subsequent to amino acid 455, numbered according to SEQ ID NO: 138. In some embodiments, the amino acid sequence is present immediately subsequent to amino acid 453, numbered according to SEQ ID NO: 981, 4, or 36. In some embodiments, the amino acid sequence is present immediately subsequent to amino acid 453, numbered according to SEQ ID NO: 138. In some embodiments, the amino acid sequence replaces 1, 2, 3, 4, 5, 6, 7, 8, 9, 10, 11, or all of amino acids 499 (e.g., K499), 450 (e.g., T450). 451 (e.g., 1451). 452 (e.g., N452), 453 (e.g., G453), 454 (e.g., S454), 455 (e.g., G455), 456 (e.g., Q456). 457 (e.g., N457), 458 (e.g., Q458), 459 (e.g., Q459), and 460 (e.g., T460), numbered according to SEQ ID NO: 138.

In some embodiments, the AAV capsid variant comprises an amino acid sequence comprising at least one, at least two, or at least three, but no more than four modifications, relative to the amino acid sequence of SPHSKA (SEQ ID NO: 941). In some embodiments, the AAV capsid variant comprises an amino acid sequence comprising at least one, at least two, or at least three, but no more than four different amino acids from the amino acid sequence of SPHSKA (SEQ ID NO: 941).

In some embodiments, the AAV capsid variant comprises an amino acid sequence comprising at least one, at least two, or at least three, but no more than four modifications, relative to the amino acid sequence of HDSPHK (SEQ ID NO: 2). In some embodiments, the AAV capsid variant comprises an amino acid sequence comprising at least one, at least two, or at least three, but no more than four different amino acids that relative to the amino acid sequence of HDSPHK (SEQ ID NO: 2).

In some embodiments, the AAV capsid variant, comprises an amino acid sequcnce of provided in Table 1, 2A, 2B, or 20-26. In some embodiments, the amino acid sequence comprises any one of SEQ ID NOs: 945-980 or 985-986. In some embodiments, the AAV capsid variant comprises the amino acid sequence of any of SEQ ID NOs: 2, 200, 201, 941, 943, 204, 208, 404, or 903-909. In some embodiments, the AAV capsid variant comprises the anino acid sequence of SEQ ID NO: 941. In some embodiments, the AAV capsid variant comprises the amino acid sequence of SEQ ID NO: 2. In some embodiments, the AAV capsid variant comprises the amino acid sequence of SEQ ID NO: 943. In some embodiments, the AAV capsid variant comprises the amino acid sequence of SEQ ID NO: 3589. In some embodiments, the AAV capsid variant comprises the amino acid sequence of SEQ ID NO: 1754. In some embodiments, the amino acid sequence is present in loop IV. In some embodiments, the amino acid sequence is present immediately subsequent to amino acid 448, relative to a reference sequence of SEQ ID NO: 138. In some embodiments, the amino acid sequence replaces amino acids 449460 (e.g., K449, T450, 1451, N452, G453, S454, G455, Q456, N457, Q458, Q459, and T460), numbered relative to SEQ ID NO: 138. In some embodiments, the amino acid sequence is present immediately subsequent to amino acid 448 and replaces amino acids 449-460 (e.g., K449, T450, 1451, N452, G453, S454, G455, Q456, N457, Q458, Q459, and T460), numbered relative to SEQ ID NO: 138. In some embodiments, the amino acid sequence is present immediately subsequent to amino acid 449, relative to a reference sequence of SEQ ID NO: 138. In some embodiments, the amino acid sequence replaces amino acids 450460 (e.g., T450, 1451, N452, G453, S454, G455, Q456, N457, Q458, Q459, and T460), numbered relative to SEQ ID NO: 138. In some embodiments, the amino acid sequence is present immediately subsequent to amino acid 449, and replaces amino acids 450-460 (e.g., T450, 1451, N452, G453, S454, G455, Q456, N457, Q458, Q459, and T460), numbered relative to SEQ ID NO: 138. In some embodiments, the amino acid sequence is present immediately subsequent to amino acid 450, relative to a reference sequence of SEQ ID NO: 138. In some embodiments, the amino acid sequence replaces amino acids 451-460 (e.g., 1451, N452. G453, S454, G455, Q456, N457, Q458, Q459, and T460), numbered relative to SEQ ID NO: 138. In some embodiments, the amino acid sequence is present immediately subsequent to amino acid 450 and replaces amino acids 451-460 (e.g., I451, N452, G453, S454, G455, Q456, N457, Q458, Q459, and T460), numbered relative to SEQ ID NO: 138. In some embodiments, the amino acid sequence is present imunediately subsequent to amino acid 451, relative to a reference sequence of SEQ ID NO: 138. In some embodiments, the amino acid sequence replaces amino acids 452-460 (e.g., N452, G453, S454, G455, Q456, N457, Q458, Q459, and T460), numbered relative to SEQ ID NO: 138. In some embodiments, the amino acid sequence is present immediately subsequent to amino acid 451 and replaces amino acids 452-460 (e.g., N452, G453, S454, G455, Q456, N457, Q458, Q459, and T460), numbered relative to SEQ ID NO: 138. In some embodiments, the amino acid sequence is present immediately subsequent to amino acid 452, relative to a reference sequence of SEQ ID NO: 138. In some embodiments, the amino acid sequence replaces amino acids 453-460 (e.g., G453, S454, G455, Q456, N457, Q458, Q459, and T460), numbered relative to SEQ ID NO: 138. In some embodiments, the amino acid sequence is present immediately subsequent to amino acid 452, and replaces amino acids 453-460 (e.g., G453, 5454, G455, Q456, N457, Q458, Q459, and T460), numbered relative to SEQ ID NO: 138. In some embodiments, the amino acid sequence is present immediately subsequent to amino acid 453, relative to a reference sequence of SEQ ID NO: 138. In some embodiments, the amino acid sequence replaces amino acids 454 and 455 (e.g., S454 and G455), numbered according to SEQ ID NO: 138. In some embodiments, the amino acid sequence is present immediately subsequent to amino acid 453, and replaces amino acids 454 and 455 (e.g., S454 and G455), numbered according to SEQ ID NO: 138. In some embodiments, the amino acid sequence replaces amino acids 454460 (e.g., S454, G455, Q456, N457, Q458, Q459, and T460), numbered relative to SEQ ID NO: 138. In some embodiments, the amino acid sequence is present immediately subsequent to amino acid 453, and replaces amino acids 454-460 (e.g., S454, G455, Q456, N457, Q458, Q459, and T460), numbered relative to SEQ ID NO: 138. In some embodiments, the amino acid sequence is present immediately subsequent to amino acid 454, relative to a reference sequence of SEQ ID NO: 138. In some embodiments, the amino acid sequence is present immediately subsequent to amino acid 454, relative to a reference sequence of SEQ ID NO: 981. In some embodiments, the amino acid sequence replaces amino acids 455-460 (e.g., amino acids G455, Q456. N457, Q458, Q459, and T460), numbered relative to SEQ ID NO: 138. In some embodiments, the amino acid sequence is present immediately subsequent to amino acids 454, and replaces amino acids 455-460 (e.g., amino acids G455, Q456, N457, Q458, Q459, and T460), numbered relative to SEQ ID NO: 138. In some embodiments, the amino acid sequence is present immediately subsequent to amino acid 455, relative to a reference sequence of SEQ ID NO: 138. In some embodiments, the amino acid sequence is present immediately subsequent to amino acid 455, relative to a reference sequence of SEQ ID NO: 982. In some embodiments, the amino acid sequence replaces amino acids 456-460 (e.g., Q456, N457, Q458, Q459, and T460), numbered relative to SEQ ID NO: 138. In some embodiments, the amino acid sequence is present immediately subsequent to amino acid 455, and replaces amino acids 456460 (e.g., Q456, N457, Q458, Q459, and T460), numbered relative to SEQ ID NO: 138.

In some embodiments, the AAV capsid variant (e.g., an AAV capsid variant described herein), comprises an amino acid sequence encoded by the nucleotide sequence of SEQ ID NO: 942 or 944, or a nucleotide sequence substantially identical (e.g., having at least 70%, at least 75%, at least 80%, at least 85%, at least 90%, at least 92%, at least 95%, at least 97%, at least 98%, or at least 99% sequence identity) thereto. In some embodiments, the AAV capsid variant described herein comprises an amino acid sequence encoded by the nucleotide sequence of SEQ ID NO: 3 or 942, or a nucleotide sequence comprising at least one, at least two, at least three, at least four, at least five, at least six, or at least seven modifications, but no more than ten modifications, relative to the nucleotide sequence of SEQ ID NO: 3 or 942. In some embodiments, the AAV capsid variant comprises an amino acid sequence encoded by a nucleotide sequence comprising at least one, at least two, at least three, at least four, at least five, at least six, or at least seven, but no more than ten different nucleotides relative to the nucleotide sequence of SEQ ID NO: 3 or 942.

In some embodiments, the nucleotide sequence encoding the AAV capsid variant (e.g., an AAV capsid variant described herein), comprises the nucleotide sequence of SEQ ID NO: 942, or a nucleotide sequence substantially identical (e.g., having at least 70%, at least 75%, at least 80%, at least 85%, at least 90%, at least 92%, at least 95%, at least 97%, at least 98%, or at least 99% sequence identity) thereto. In some embodiments, the nucleic acid sequence encoding the AAV capsid variant comprises a nucleotide sequence comprising at least one, at least two, at least three, at least four, at least five, at least six, or at least seven modifications, but no more than ten modifications, relative to the nucleotide sequences of SEQ ID NO: 942. In some embodiments, the nucleotide sequence encoding an AAV capsid variant comprises a nucleotide sequence comprising at least one, at least two, at least three, at least four, at least five, at least six, or at least seven, but no more than ten different nucleotides, relative to the nucleotide sequence of SEQ ID NO: 942.

In some embodiments, the nucleotide sequence encoding the AAV capsid variant (e.g., an AAV capsid variant described herein), comprises the nucleotide sequence of SEQ ID NO: 3, or a nucleotide sequence substantially identical (e.g., having at least 70%, at least 75%, at least 80%, at least 85%, at least 90%, at least 92%, at least 95%, at least 97%, at least 98%, or at least 99% sequence identity) thereto. In some embodiments, the nucleic acid sequence encoding the AAV capsid variant comprises a nucleotide sequence comprising at least one, at least two, at least three, at least four, at least five, at least six, or at least seven modifications, but no more than ten modifications, relative to the nucleotide sequences of SEQ ID NO: 3. In some embodiments, the nucleotide sequence encoding an AAV capsid variant comprises a nucleotide sequence comprising at least one, at least two, at least three, at least four, at least five, at least six, or at least seven, but no more than ten different nucleotides relative to the nucleotide sequence of SEQ ID NO: 3.

In some embodiments, the nucleotide sequence encoding the AAV capsid variant (e.g., an AAV capsid variant described herein), comprises the nucleotide sequence of SEQ ID NO: 5, or a nucleotide sequence substantially identical (e.g., having at least 70%, at least 75%, at least 80%, at least 85%, at least 90%, at least 92%, at least 95%, at least 97%, at least 98%, or at least 99% sequence identity) thereto. In some embodiments, the nucleic acid sequence encoding the AAV capsid variant comprises a nucleotide sequence comprising at least one, at least two, at least three, at least four, at least five, at least six, or at least seven modifications, but no more than ten modifications, relative to the nucleotide sequences of SEQ ID NO: 5. In some embodiments, the nucleotide sequence encoding an AAV capsid variant comprises a nucleotide sequence comprising at least one, at least two, at least three, at least four, at least five, at least six, or at least seven, but no more than ten different nucleotides relative to the nucleotide sequence of SEQ ID NO: 5.

In some embodiments, the nucleotide sequence encoding the AAV capsid variant (e.g., an AAV capsid variant described herein), comprises the nucleotide sequence of SEQ ID NO: 12, or a nucleotide sequence substantially identical (e.g., having at least 70%, at least 75%, at least 80%, at least 85%, at least 90%, at least 92%, at least 95%, at least 97%, at least 98%, or at least 99% sequence identity) thereto. In some embodiments, the nucleic acid sequence encoding the AAV capsid variant comprises a nucleotide sequence comprising at least one, at least two, at least three, at least four, at least five, at least six, or at least seven modifications, but no more than ten modifications, relative to the nucleotide sequences of SEQ ID NO: 12. In some embodiments, the nucleotide sequence encoding an AAV capsid variant comprises a nucleotide sequence comprising at least one, at least two, at least three, at least four, at least five, at least six, or at least seven, but no more than ten different nucleotides relative to the nucleotide sequence of SEQ ID NO: 12.

In some embodiments, an AAV capsid variant comprises the amino acid sequence of SPHSKA (SEQ ID NO: 941), wherein the amino acid sequence is present immediately subsequent to amino acid 455, relative to a reference sequence of SEQ ID NO: 138. In some embodiments, an AAV capsid variant comprises the amino acid sequence of SPHSKA (SEQ ID NO: 941), wherein the amino acid sequence is present immediately subsequent to amino acid 455, relative to a reference sequence of SEQ ID NO: 981.

In some embodiments, an AAV capsid variant comprises the amino acid sequence of HDSPHK (SEQ ID NO: 2), wherein the amino acid sequence is present immediately subsequent to amino acid 453, relative to a reference sequence of SEQ ID NO: 138. In some embodiments, an AAV capsid variant comprises the amino acid sequence of HDSPHK (SEQ ID NO: 2), wherein the amino acid sequence is present immediately subsequent to amino acid 453, relative to a reference sequence of SEQ ID NO: 982.

In some embodiments, an AAV capsid variant comprises (i) the amino acid sequence of HDSPHSKA (SEQ ID NO: 4486), which is present immediately subsequent to amino acid 453; and (ii) a deletion of amino acids SG at amino acid 454 and 455; wherein (i) and (ii) are numbered according to SEQ ID NO: 138.

In some embodiments, an AAV capsid variant comprises an amino acid other than S at amino acid 454 and/or an amino acid other than G at amino acid 455, numbered according to SEQ ID NO: 138. In some embodiments, the AAV capsid variant comprises the amino acid H at amino acid 454 and the amino acid D at amino acid 455, numbered according to SEQ ID NO: 138. In some embodiments, the AAV capsid variant further comprises the amino acid sequence of SPHSKA (SEQ ID NO: 941). In some embodiments, the AAV capsid variant comprises: (i) the amino acid H at amino acid 454 and the amino acid D at amino acid 455, and (ii) the amino acid sequence SPHSKA (SEQ ID NO: 941), wherein the amino acid sequence of SPHKSG (SEQ ID NO: 946) is present immediately subsequent to amino acid 455, wherein (i) and (ii) are numbered according to SEQ ID NO: 138.

In some embodiments, an AAV capsid variant comprises a modification, e.g., substitution, relative to SEQ ID NO: 138. In some embodiments, the AAV capsid variant comprises a modification, e.g., substitution, at amino acid S454 and/or G455, numbered relative to SEQ ID NO: 138. In some embodiments, the AAV capsid variant comprises a S454H substitution and/or G455D substitution, numbered relative to SEQ ID NO: 138. In some embodiments, the AAV capsid variant comprises a S454H substitution and a G455D substitution, numbered relative to SEQ ID NO: 138. In some embodiments, the AAV capsid variant further comprises the amino acid sequence of SPHSKA (SEQ ID NO: 941). In some embodiments, the AAV capsid variant comprises: (i) a S454H substitution and a G455D substitution, and (ii) the amino acid sequence SPHKSG (SEQ ID NO: 946), wherein the amino acid sequence of SPHSKA (SEQ ID NO: 941) is present immediately subsequent to amino acid 455, wherein (i) and (ii) are numbered according to SEQ ID NO: 138.

In some embodiments, the AAV capsid variant further comprises one, two, or all of an amino acid other than T at amino acid 450 (e.g., S, Y, or G), an amino acid other than I at amino acid 451 (e.g., M or L), and/or an amino acid other than N at amino acid 452 (e.g., S), relative to a reference sequence of SEQ ID NO: 138. In some embodiments, the AAV capsid variant further comprises an S at amino acid 450 and an M at amino acid 451, relative to a reference sequence of SEQ ID NO: 138. In some embodiments, the AAV capsid variant further comprises a Y at amino acid 450, an L at amino acid 451, and an S at amino acid 452, relative to a reference sequence of SEQ ID NO: 138. In some embodiments, the AAV capsid variant further comprises a G at amino acid 450, an L at amino acid 451, and an S at amino acid 452, relative to a reference sequence of SEQ ID NO: 138.

In some embodiments, the AAV capsid variant further comprises one, two, three, four, or all of an amino acid other than Q at amino acid 456 (e.g., R or L), N at amino acid 457 (e.g., H, K, or R), Q at amino acid 458 (e.g., R or T), Q at amino acid 459 (H), and/or T at amino acid 460 (N or S), relative to a reference sequence of SEQ ID NO: 138. In some embodiments, the AAV capsid variant further comprises an R at amino acid 456, relative to a reference sequence of SEQ ID NO: 138. In some embodiments, the AAV capsid variant further comprises an L at amino acid 456, relative to a reference sequence of SEQ ID NO: 138. In some embodiments, the AAV capsid variant further comprises an H at amino acid 457 and an R at amino acid 458, relative to a reference sequence of SEQ ID NO: 138. In some embodiments, the AAV capsid variant further comprises a K at amino acid 457 and an N at amino acid 460, relative to a reference sequence of SEQ ID NO: 138. In some embodiments, the AAV capsid variant further comprises a T at amino acid 458, an H at amino acid 459, and an S at amino acid 460, relative to a reference sequence of SEQ ID NO: 138. In some embodiments, the AAV capsid variant further comprises an R at amino acid 456, an R at amino acid 457, and an R at amino acid 458, relative to a reference sequence of SEQ ID NO: 138.

In some embodiments, an AAV capsid variant comprises an amino acid other than I at amino acid 451, an amino acid other than N at amino acid 452, and an amino acid other than G at amino acid 453, numbered according to SEQ ID NO: 138 or 981. In some embodiments, the AAV capsid variant comprises E at amino acid 451. R at amino acid 452, and V at amino acid 453, numbered according to SEQ ID NO: 138 or 981.

In some embodiments, the AAV capsid variant comprises the substitutions 1451E, N452R, and G453V, numbered according to SEQ ID NO: 138 or 981.

In some embodiments, the AAV capsid variant comprises the amino acid sequence of SPHSKA (SEQ ID NO: 941), wherein the amino acid sequence is present immediately subsequent to amino acid 455 and wherein the AAV capsid variant comprises the E at amino acid 451, R at amino acid 452, and V at amino acid 453, numbered according to SEQ ID NO: 138 or 981. In some embodiments, the AAV capsid variant comprises the substitutions 1451E, N452R, and G453V, and further comprises the amino acid sequence of SPHSKA (SEQ ID NO: 941), wherein the amino acid sequence is present immediately subsequent to amino acid 455, numbered according to SEQ ID NO: 138 or 981. In some embodiments, the AAV capsid variant comprises the amino acid sequence of ERVSGSPHSKA (SEQ ID NO: 6399), wherein the amino acid sequence is present immediately subsequent to amino acid 449 and replaces amino acids 450-455, numbered according to SEQ ID NO: 138. In some embodiments, the AAV capsid variant comprises the amino acid sequence of KTERVSGSPHSKAQNQQT (SEQ ID NO: 3589), wherein the amino acid sequence is present immediately subsequent to amino acid 448 and replaces amino acids 449-460, numbered according to SEQ ID NO: 138.

In some embodiments, an AAV capsid variant comprises: (i) the amino acid sequence of SPHSKA (SEQ ID NO: 941), wherein the amino acid sequence is present immediately subsequent to position 455, numbered according to SEQ ID NO: 138, 981, or 4; and (ii) one or both of E at position 451 and/or V at position 453, numbered according to the amino acid sequence of SEQ ID NO: 4, 138, or 981. In some embodiments, the AAV capsid variant comprises: (i) the amino acid sequence of SPHSKA (SEQ ID NO: 941), wherein the amino acid sequence is present immediately subsequent to position 455, numbered according to SEQ ID NO: 138, 981, or 4; and (ii) one or both of E at position 451 and/or V at position 453, numbered according to the amino acid sequence of SEQ ID NO: 4, 138, or 981, wherein the AAV capsid variant is an AAV9 variant.

In some embodiments, the AAV capsid variant comprises: (i) the amino acid sequence of SPHSKA (SEQ ID NO: 941), wherein the amino acid sequence is present immediately subsequent to position 455, numbered according to SEQ ID NO: 138, 981, or 4; and (ii) E at position 451 and V at position 453, numbered according to the amino acid sequence of SEQ ID NO: 4, 138, or 981. In some embodiments, the AAV capsid variant comprises: (i) the amino acid sequence of SPHSKA (SEQ ID NO: 941), wherein the amino acid sequence is present immediately subsequent to position 455, numbered according to SEQ ID NO: 138, 981, or 4; and (ii) E at position 451 and V at position 453, numbered according to the amino acid sequence of SEQ ID NO: 4, 138, or 981, wherein the AAV capsid variant is an AAV9 variant.

In some embodiments, the AAV capsid variant comprises: (i) the amino acid sequence of SPHSKA (SEQ ID NO: 941), wherein the amino acid sequence is present at amino acids 456-461, numbered according to SEQ ID NO: 4, 138, or 981 (i.e., at a sequence position corresponding to that in SEQ ID NO: 4, 138, or 981) and (ii) one or both of E at position 451 and/or V at position 453, numbered according to the amino acid sequence of SEQ ID NO: 4, 138, or 981 (i.e., at a sequence position corresponding to that in SEQ ID NO: 4, 138, or 981). In some embodiments, the AAV capsid variant comprises: (i) the amino acid sequence of SPHSKA (SEQ ID NO: 941), wherein the amino acid sequence is present at amino acids 456-461, numbered according to SEQ ID NO: 4, 138, or 981 and (ii) one or both of E at position 451 and/or V at position 453, numbered according to the amino acid sequence of SEQ ID NO: 4, 138, or 981, wherein the AAV capsid variant is an AAV9 variant.

In some embodiments, the AAV capsid variant comprises: (i) the amino acid sequence of SPHSKA (SEQ ID NO: 941), wherein the amino acid sequence is present at amino acids 456-461, numbered according to SEQ ID NO: 4, 138, or 981 (i.e., at a sequence position corresponding to that in SEQ ID NO: 4, 138, or 981); and (ii) E at position 451 and V at position 453, numbered according to the amino acid sequence of SEQ ID NO: 4, 138, or 981 (i.e., at a sequence position corresponding to that in SEQ ID NO: 4, 138, or 981). In some embodiments, the AAV capsid variant comprises: (i) the amino acid sequence of SPHSKA (SEQ ID NO: 941), wherein the amino acid sequence is present at amino acids 456-461, numbered according to SEQ ID NO: 4, 138, or 981, and (ii) E at position 451 and V at position 453, numbered according to the amino acid sequence of SEQ ID NO: 4, 138, or 981, wherein the AAV capsid variant is an AAV9 variant.

In some embodiments, the AAV capsid variant comprises: (i) the amino acid sequence of SPHSKA (SEQ ID NO: 941), wherein the amino acid sequence is present at amino acids 456-461, numbered according to SEQ ID NO: 36, 138, or 981 (i.e., at a sequence position corresponding to that in SEQ ID NO: 36, 138, or 981), and (ii) one, two, or all of E at position 451, R at position 452, and/or V at position 453, numbered according to the amino acid sequence of SEQ ID NO: 36, 138, or 981 (i.e., at a sequence position corresponding to that in SEQ ID NO: 36, 138, or 981). In some embodiments, the AAV capsid variant comprises: (i) the amino acid sequence of SPHSKA (SEQ ID NO: 941), wherein the amino acid sequence is present at amino acids 456-461, numbered according to SEQ ID NO: 36, 138, or 981 and (ii) one, two, or all of E at position 451, R at position 452, and/or V at position 453, numbered according to the amino acid sequence of SEQ ID NO: 36, 138, or 981, wherein the AAV capsid variant is an AAV9 variant.

In some embodiments, the AAV capsid variant comprises: (i) the amino acid sequence of SPHSKA (SEQ ID NO: 941), wherein the amino acid sequence is present at amino acids 456-461, numbered according to SEQ ID NO: 36, 138, or 981 (i.e., at a sequence position corresponding to that in SEQ ID NO: 36, 138, or 981); and (ii) E at position 451. R at position 452, and V at position 453, numbered according to the amino acid sequence of SEQ ID NO: 36, 138, or 981 (i.e., at a sequence position corresponding to that in SEQ ID NO: 36, 138, or 981). In some embodiments, the AAV capsid variant comprises: (i) the amino acid sequence of SPHSKA (SEQ ID NO: 941), wherein the amino acid sequence is present at amino acids 456-461, numbered according to SEQ ID NO: 36, 138, or 981 and (ii) E at position 451. R at position 452, and V at position 453, numbered according to the amino acid sequence of SEQ ID NO: 36, 138, or 981, wherein the AAV capsid variant is an AAV9 variant.

In some embodiments, an AAV capsid variant described herein comprises an amino acid other than T at amino acid 450, an amino acid other than I at amino acid 451, and an amino acid other than N at amino acid 452, numbered according to SEQ ID NO: 138 or 982. In some embodiments, the AAV capsid variant comprises A at amino acid 450, E at amino acid 451, and I at amino acid 452, numbered according to SEQ ID NO: 138 or 982. In some embodiments, the AAV capsid variant comprises the substitutions T450A, 1451E, and N4521, numbered according to SEQ ID NO: 138 or 982.

In some embodiments, the AAV capsid variant comprises the amino acid sequence of HDSPHK (SEQ ID NO: 2), which is present immediately subsequent to amino acids 453, and further comprises A at amino acid 450, E at amino acid 451, and I at amino acid 452, all numbered according to SEQ ID NO: 138 or 982. In some embodiments, the AAV capsid variant comprises the substitutions T450A, I451E, and N452I, and further comprises the amino acid sequence HDSPHK (SEQ ID NO: 2) present immediately subsequent to amino acid 453, all numbered according to SEQ ID NO: 138 or 982. In some embodiments, the AAV capsid variant comprises the amino acid sequence of AEIGHDSPHKSG (SEQ ID NO: 6400), wherein the amino acid sequence is present immediately subsequent to amino acid 449 and replaces amino acids 450-455, numbered according to SEQ ID NO: 138.

In some embodiments, the AAV capsid variant comprises the amino acid sequence of KAEIGHDSPHKSGQNQQT (SEQ ID NO: 1754), wherein the amino acid sequence is present immediately subsequent to amino acid 448 and replaces amino acids 449-460, numbered according to SEQ ID NO: 138.

In some embodiments, the AAV capsid variant, further comprises a substitution at amino acid K449, e.g., a K449R substitution, numbered according to SEQ ID NO: 138. In some embodiments, the AAV capsid variant, further comprises an amino acid other than K at amino acid 449 (e.g., R), relative to a reference sequence of SEQ ID NO: 138. In some embodiments, the AAV capsid variant comprises an R at amino acid 449, relative to a reference sequence of SEQ ID NO: 138. In some embodiments, the AAV capsid variant further comprises a modification, e.g., an insertion, substitution, and/or deletion in loop I, II, VI, and/or VIII.

In some embodiments, the AAV capsid variant, further comprises an amino acid sequence comprising at least one, at least two, or at least three modifications, but not more than 30, not more than 20, or not more than 10 modifications, of the amino acid sequence of SEQ ID NO: 138. In some embodiments, the AAV capsid variant further comprises an amino acid sequence comprising at least one, at least two, or at least three, but not more than 30, not more than 20, or not more than 10 amino acids that differ from the amino acid sequence of SEQ ID NO: 138. In some embodiments, the AAV capsid variant further comprises the amino acid sequence of SEQ ID NO: 138, or an amino acid sequence with at least 70% (e.g., at least 80%, at least 85%, at least 90, at least 95%, at least 96%, at least 97%, at least 98%, or at least 99%) sequence identity thereto.

In some embodiments, the AAV capsid variant further comprises (a) a VP1 protein comprising the amino acid sequence of SEQ ID NO: 982, 36, or 4; (b) a VP2 protein comprising amino acids 138-742 of SEQ ID NO: 982, 36, or 4; (c) a VP3 protein comprising amino acids 203-742 of SEQ ID NO: 982, 36, or 4; or (d) an amino acid sequence with at least 70% (e.g., at least 70%, at least 75%, at least 80%, at least 85%, at least 90, at least 95%, at least 96%, at least 97%, at least 98%, or at least 99%) sequence identity to any of the amino acid sequences in (a)-(c), an amino acid sequence comprising at least one, at least two, or at least three, but not more than 30, not more than 20, or not more than 10 different amino acids relative to any of the amino acid sequences in (a)-(c), or an amino acid sequence comprising at least one, at least two, or at least three modifications, but not more than 30, not more than 20, or not more than 10 modifications, relative to any of the amino acid sequences in (a)-(c).

In some embodiments, the AAV capsid variant further comprises an amino acid sequence encoded by a nucleotide sequence that is at least 70% (e.g., at least 70%, at least 75%, at least 80%, at least 85%, at least 90, at least 95%, at least 96%, at least 97%, at least 98%, or at least 99%) identical to SEQ ID NO: 137. In some embodiments, the AAV capsid variant comprises an amino acid sequence encoded by a nucleotide sequence comprising at least one, at least two, or at least three modifications, but not more than 30, not more than 20, or not more than 10 modifications, relative to the nucleotide sequence of SEQ ID NO: 137. In some embodiments, the AAV capsid variant comprises an amino acid sequence encoded by a nucleotide sequence comprising at least one, at least two, or at least three, but not more than 30, not more than 20, or not more than 10 different nucleotides, relative to the amino acid sequence of SEQ ID NO: 137.

In some embodiments, the nucleotide sequence encoding the AAV capsid variant further comprises a nucleotide sequence that is at least 70% (e.g., at least 70%, at least 75%, at least 80%, at least 85%, at least 90, at least 95%, at least 96%, at least 97%, at least 98%, or at least 99%) identical to SEQ ID NO: 137. In some embodiments, the nucleotide sequence encoding the AAV capsid variant comprises a nucleotide sequence comprising at least one, at least two, or at least three modifications, but not more than 30, not more than 20, or not more than 10 modifications, relative to the nucleotide sequence of SEQ ID NO: 137. In some embodiments, the nucleotide sequence encoding the AAV capsid variant comprises a nucleotide sequence comprising at least one, at least two, or at least three, but not more than 30, not more than 20, or not more than 10 different nucleotides, relative to the amino acid sequence of SEQ ID NO: 137.

In some embodiments, an AAV capsid variant of the present disclosure comprises an amino acid sequence as described herein, e.g., an amino acid sequence of an AAV capsid variant of TTM-001 or TTM-002, e.g., as described in Tables 3 and 4. In some embodiments, an AAV capsid variant of the present disclosure comprises an amino acid sequence as described herein, e.g., an amino acid sequence of an AAV capsid variant of TTM-003, TTM-004, TTM-005, TTM-006, TTM-007, TTM-008, TTM-009, TTM-010, TTM-011, TTM-012, TTM-013, TTM-014, TTM-015, TTM-016, TTM-017, TTM-018, TTM-019, TTM-020, TTM-021, TTM-022, TTM-023, TM-024, TTM-025, TM-026, or TTM-027, e.g., as described in Table 4. In some embodiments, the AAV capsid variant comprises an amino acid sequence of SEQ ID NO: 36 (TTM-003: comprising a peptide of SEQ ID NO: 3589), SEQ ID NO: 39 (TTM-006; comprising a peptide of SEQ ID NO: 3241), or SEQ ID NO: 4 (TTM-027: comprising a peptide of SEQ ID NO: 3272).

In some embodiments, an AAV capsid variant comprises a VP1. VP2, and/or VP3 protein comprising an amino acid sequence described herein, e.g., an amino acid sequence of an AAV capsid variant of TTM-001 or TTM4002, e.g., as described in Tables 3 and 4. In some embodiments, an AAV capsid variant comprises a VP1, VP2, and/or VP3 protein comprising an amino acid sequence described herein, e.g., an amino acid sequence of an AAV capsid variant of TTM-003, TTM-004, TTM-005, TTM-006, TTM-007, TTM-008, TTM-009, TTM-010, TTM-001, TTM-012, TTM-13, TTM-014, TTM-015, TTM-016, TTM-017, TTM-018, TTM-019, TTM-020, TTM-021, TTM-022, TTM-023, TTM-024, TTM-025, TTM-026, or TTM-027, e.g., as described in Table 4.

In some embodiments, an AAV capsid variant comprises an amino acid sequence encoded by a nucleotide sequence as described herein, e.g., a nucleotide sequence of an AAV capsid variant of TTM-01 or TTM-002, e.g., as described in Tables 3 and 5. In some embodiments, an AAV capsid variant comprises an amino acid sequence encoded by a nucleotide sequence as described herein, e.g., a nucleotide sequence of an AAV capsid variant of TTM-003, TTM-004, TTM-005, TTM-006, TTM-007, TTM-008, TTM-009, TTM-010, TTM-101, TTM-012, TTM-013, TTM-014, TTM-015, TTM-016, TTM-017, TTM-018, TTM-019, TTM420, TTM4021, TTM-022, TTM423, TTM-024, TTM-025, TTM-026, or TTM-027, e.g., as described in Table 5.

In some embodiments, a polynucleotide or nucleic acid encoding an AAV capsid variant, of the present disclosure comprises a nucleotide sequence described herein, e.g., a nucleotide sequence of an AAV capsid variant of TTM-(001 or TTM-002, e.g., as described in Tables 3 and 5. In some embodiments, a polynucleotide or nucleic acid encoding an AAV capsid variant, of the present disclosure comprises a nucleotide sequence described herein, e.g., a nucleotide sequence of an AAV capsid variant of TTM-003, TTM-004, TTM-005, TIM-006, TTM-007, TTM-08, TTM-009, TTM-010, TTM-11, TTM-012, TTM-013, TTM-014, TTM-015, TIM-016, TTM-017, TTM-018, TTM-019, TTM4020, TTM-021, TTM-022, TTM-023, TTM4024, TTM-025, TTM-026, or TTM4027, e.g., as described in Table 5.

TABLE 3
Exemplary full length capsid sequences

| | | VP1 | Peptide | |
|---|---|---|---|---|
| | VP1 | (amino | (amino | Peptide |
| | DNA SEQ | acid) SEQ | acid) SEQ | DNA SEQ |
| Name | ID NO: | ID NO: | ID NO: | ID NO: |
| TTM-001 | 983 | 981 | 941 | 942 |
| TTM-002 | 984 | 982 | 2 | 3 |

TABLE 4
Exemplary full length capsid amino acid sequences

| | SEQ | |
|---|---|---|
| Name and | ID | |
| Annotation | NO: | Amino Acid Sequence |
| TTM-001 | 981 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTINGSGSPHSKAQNQQTLKFSVAGPSNMAVQGRNYIPGPSYRQQRVSTTVTQNNN |
| 742 aa | | SEFAWPGASSWALNGRNSLMNPGPAMASHKEGEDRFFPLSGSLIFGKQGTGRDNVD |
| | | ADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-002 | 982 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 454 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 453); | | KTINGHDSPHKSGQNQQTLKFSVAGPSNMAVQGRNYIPGPSYRQQRVSTTVTQNNN |
| 742 aa | | SEFAWPGASSWALNGRNSLMNPGPAMASHKEGEDRFFPLSGSLIFGKQGTGRDNVD |
| | | ADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-003 | 36 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTERVSGSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 451,452, and | | DVYLQGPIWAKIPHTDGNFHPSPLMGGFGMKHPPPQILIKNTPVPADPPTAFNKDK |
| 453 underlined: | | LNSFITQYSTGQVSVEIEWELQKENSKRWNPEIQYTSNYYKSNNVEFAVNTEGVYS |
| 742 aa | | EPRPIGTRYLTRNL |
| TTM-004 | 37 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 454 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 453); | | KAEIGHDSPHKSGQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 450, 451, and | | DVYLQGPIWAKIPHTDGNFHPSPLMGGFGMKHPPPQILIKNTPVPADPPTAFNKDK |
| 452 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-005 | 38 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTIIGSGSPHSKAQNRHTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 452, 464, and | | DVYLQGPIWAKIPHTDGNFHPSPLMGGFGMKHPPPQILIKNTPVPADPPTAFNKDK |
| 465 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-006 | 39 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTEKMSGSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 451, 452, and | | DVYLQGPIWAKIPHTDGNFHPSPLMGGFGMKHPPPQILIKNTPVPADPPTAFNKDK |
| 453 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-007 | 40 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KEINGRGSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 450 and 454 | | DVYLQGPIWAKIPHTDGNFHPSPLMGGFGMKHPPPQILIKNTPVPADPPTAFNKDK |
| underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-008 | 41 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTFNGSGSPHSKAPNLQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 451, 462, and | | DVYLQGPIWAKIPHTDGNFHPSPLMGGFGMKHPPPQILIKNTPVPADPPTAFNKDK |
| 464 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-009 | 42 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTEKTSGSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 451, 452, and | | DVYLQGPIWAKIPHTDGNFHPSPLMGGFGMKHPPPQILIKNTPVPADPPTAFNKDK |
| 453 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-010 | 43 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTMNGHDSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 451, 454, and | | DVYLQGPIWAKIPHTDGNFHPSPLMGGFGMKHPPPQILIKNTPVPADPPTAFNKDK |
| 455 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-011 | 44 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTIDGHDSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 452, 454, and | | DVYLQGPIWAKIPHTDGNFHPSPLMGGFGMKHPPPQILIKNTPVPADPPTAFNKDK |
| 455 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-012 | 45 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTNNGHDSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 452, 454, and | | DVYLQGPIWAKIPHTDGNFHPSPLMGGFGMKHPPPQILIKNTPVPADPPTAFNKDK |
| 455 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-013 | 46 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTQRKSGSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 451, 452, and | | DVYLQGPIWAKIPHTDGNFHPSPLMGGFGMKHPPPQILIKNTPVPADPPTAFNKDK |
| 453 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-014 | 47 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTINGSGSPHSKAQARKTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 463, 464, and | | DVYLQGPIWAKIPHTDGNFHPSPLMGGFGMKHPPPQILIKNTPVPADPPTAFNKDK |
| 465 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-015 | 48 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KYIVGSGSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 450 and 452 | | DVYLQGPIWAKIPHTDGNFHPSPLMGGFGMKHPPPQILIKNTPVPADPPTAFNKDK |
| underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-016 | 49 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTISKRGSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 452, 453, and | | DVYLQGPIWAKIPHTDGNFHPSPLMGGFGMKHPPPQILIKNTPVPADPPTAFNKDK |
| 454 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-017 | 50 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455) | | KGLGGSGSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 450, 451, and | | DVYLQGPIWAKIPHTDGNFHPSPLMGGFGMKHPPPQILIKNTPVPADPPTAFNKDK |
| 452 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-018 | 51 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTINGHDSPHDKAQNLQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 454, 455, and | | DVYLQGPIWAKIPHTDGNFHPSPLMGGFGMKHPPPQILIKNTPVPADPPTAFNKDK |
| 464 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-019 | 52 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455) | | KTVNGHDSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 451, 454, and | | DVYLQGPIWAKIPHTDGNFHPSPLMGGFGMKHPPPQILIKNTPVPADPPTAFNKDK |
| 455 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-020 | 53 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTINGHDSPHSKALNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 454, 455, and | | DVYLQGPIWAKIPHTDGNFHPSPLMGGFGMKHPPPQILIKNTPVPADPPTAFNKDK |
| 462 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-021 | 54 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTINGHDSPHSKAQNQQSLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 454, 455, and | | DVYLQGPIWAKIPHTDGNFHPSPLMGGFGMKHPPPQILIKNTPVPADPPTAFNKDK |
| 466 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-022 | 55 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455) | | KTINGHDSPHSKAQNQQILKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 454, 455, and | | DVYLQGPIWAKIPHTDGNFHPSPLMGGFGMKHPPPQILIKNTPVPADPPTAFNKDK |
| 466 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-023 | 56 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTINGSGSPHFTRQNQQTLKFSVAGPSNMAVQGRNYIPGPSYRQQRVSTTVTQNNN |
| 742 aa | | SEFAWPGASSWALNGRNSLMNPGPAMASHKEGEDRFFPLSGSLIFGKQGTGRDNVD |
| | | ADKVMITNEEEIKTTNPVATESYGQVATNHQSAQAQAQTGWVQNQGILPGMVWQDR |
| | | DVYLQGPIWAKIPHTDGNFHPSPLMGGFGMKHPPPQILIKNTPVPADPPTAFNKDK |
| | | LNSFITQYSTGQVSVEIEWELQKENSKRWNPEIQYTSNYYKSNNVEFAVNTEGVYS |
| | | EPRPIGTRYLTRNL |
| TTM-024 | 57 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTSNGHDSPHSKAQNQQTLKFSVAGPSNMAVQGRNYIPGPSYRQQRVSTTVTQNNN |
| modifications at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 451, 454, and | | DVYLQGPIWAKIPHTDGNFHPSPLMGGFGMKHPPPQILIKNTPVPADPPTAFNKDK |
| 455 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-025 | 58 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTINGSGSPHSLPWNQQTLKFSVAGPSNMAVQGRNYIPGPSYRQQRVSTTVTQNNN |
| modification at | | SEFAWPGASSWALNGRNSLMNPGPAMASHKEGEDRFFPLSGSLIFGKQGTGRDNVD |
| amino acid 462 | | ADKVMITNEEEIKTTNPVATESYGQVATNHQSAQAQAQTGWVQNQGILPGMVWQDR |
| underlined; | | DVYLQGPIWAKIPHTDGNFHPSPLMGGFGMKHPPPQILIKNTPVPADPPTAFNKDK |
| 742 aa | | LNSFITQYSTGQVSVEIEWELQKENSKRWNPEIQYTSNYYKSNNVEFAVNTEGVYS |
| | | EPRPIGTRYLTRNL |
| TTM-026 | 59 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455): | | KTINGHDSPHSKAQNHQTLKFSVAGPSNMAVQGRNYIPGPSYRQQRVSTTVTQNNN |
| modification at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 454, 455, and | | DVYLQGPIWAKIPHTDGNFHPSPLMGGFGMKHPPPQILIKNTPVPADPPTAFNKDK |
| 464 underlined; | | LNSFITQYSTGQVSVEIEWELQKENSKRWNPEIQYTSNYYKSNNVEFAVNTEGVYS |
| 742 aa | | EPRPIGTRYLTRNL |
| TTM-027 | 4 | MAADGYLPDWLEDNLSEGIREWWALKPGAPQPKANQQHQDNARGLVLPGYKYLGPG |
| 6mer peptide | | NGLDKGEPVNAADAAALEHDKAYDQQLKAGDNPYLKYNHADAEFQERLKEDTSFGG |
| underlined, | | NLGRAVFQAKKRLLEPLGLVEEAAKTAPGKKRPVEQSPQEPDSSAGIGKSGAQPAK |
| starts at amino | | KRLNFGQTGDTESVPDPQPIGEPPAAPSGVGSLTMASGGGAPVADNNEGADGVGSS |
| acid 456 | | SGNWHCDSQWLGDRVITTSTRTWALPTYNNHLYKQISNSTSGGSSNDNAYFGYSTP |
| (immediately | | WGYFDFNRFHCHFSPRDWQRLINNNWGFRPKRLNFKLFNIQVKEVTDNNGVKTIAN |
| subsequent to | | NLTSTVQVFTDSDYQLPYVLGSAHEGCLPPFPADVFMIPQYGYLTLNDGSQAVGRS |
| amino acid | | SFYCLEYFPSQMLRTGNNFQFSYEFENVPFHSSYAHSQSLDRLMNPLIDQYLYYLS |
| 455); | | KTENVSGSPHSKAQNQQTLKFSVAGPSNMAVQGRNYIPGPSYRQQRVSTTVTQNNN |
| modification at | | SEFAWPGASSWALNGRNSLMNPGPAMASHKEGEDRFFPLSGSLIFGKQGTGRDNVD |
| amino acids | | ADKVMITNEEEIKTTNPVATESYGQVATNHQSAQAQAQTGWVQNQGILPGMVWQDR |
| 451 and 453 | | DVYLQGPIWAKIPHTDGNFHPSPLMGGFGMKHPPPQILIKNTPVPADPPTAFNKDK |
| underlined; | | LNSFITQYSTGQVSVEIEWELQKENSKRWNPEIQYTSNYYKSNNVEFAVNTEGVYS |
| 742 aa | | EPRPIGTRYLTRNL |

TABLE 5
Exemplary full length capsid nucleic acid sequences

| | SEQ | |
|---|---|---|
| Name and | ID | |
| Annotation | NO: | NT Sequence |
| TTM-001 | 983 | ATGGCTGCCGATGGTTATCTTCCAGattggcTCGAGGACAACCTTAGTGAAGGAAT |
| 9mer | | TCGCGAGTGGTGGGCTTTGAAACCTGGAGCCCCTCAACCCAAGGCAAATCAACAAC |
| peptide | | ATCAAGACAACGCTCGAGGTCTTGTGCTTCCGGGTTACAAATACCTTGGACCCGGC |
| underlined | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGACCAATACTTGTATTActTgagt |
| | | AAaACaATTAACGGAAGCGGAAGCCCACACAGCAAAGCACAAAACCAACAGACCET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTaTAA |
| TTM-002 | 984 | ATGGCTGCCGATGGTTATCTTCCAGattggcTCGAGGACAACCTTAGTGAAGGAAT |
| 7mer | | TCGCGAGTGGTGGGCTTTGAAACCTGGAGCCCCTCAACCCAAGGCAAATCAACAAC |
| peptide | | ATCAAGACAACGCTCGAGGTCTTGTGCTTCCGGGTTACAAATACCTTGGACCCGGC |
| underlined. | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGACCAATACTTGTATTActTgagt |
| | | AAaACaATTAACGGACACGACAGCCCACACAAAAGCGGACAAAACCAACAGACCET |
| | | GAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTaTAA |
| TTM-003 | 12 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATOTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGGAGCGTGTGTCTGGTTCTCCGCATTCTAAGGCGCAGAATCAGCAGACGUT |
| | | gAAgTTttcgGTaGCtGGtCCtAGcAAcATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGGTAttTAACgaGgAActTa |
| TTM-004 | 13 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATOTTCAAATGACAACGCCTACTTCGGCTACAGCACCCCC |
| | | TGGGGGTATTTTGACTTCAACAGATTCCACTGCCACTTCTCACCACGTGACTGGCA |
| | | TCAACATTCAGGTCAAAGAGGTTACGGACAACAATGGAGTCAAGACCATCGCCAAT |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAGTAtcTGTAtTActTgagt |
| | | AAGGCGGAGATTGGTCATGATTCTCCGCATAAGTCTGGTCAGAATCAGCAGACGtT |
| | | GAAgTTttcgGTaGCtGGtCCtAGcAAcATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTAATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-005 | 14 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTATTGGTTCTGGTTCTCCGCATTCTAAGGCGCAGAATCGTCATACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-006 | 15 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGGAGAAGATGTCTGGTTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGTAttTAACgaGgAActTa |
| TTM-007 | 16 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGGAGATTAATGGTCGTGGTTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-008 | 17 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGTTTAATGGTTCTGGTTCTCCGCATTCTAAGGCGCCGAATCTGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCOTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-009 | 18 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGGAGAAGACGTCTGGTTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-010 | 19 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATGAATGGTCATGATTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-011 | 20 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTGATGGTCATGATTCTCCGCATTCTAAGGCGCAGAATCAGCAGACGtT |
| | | GAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-012 | 21 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGAATAATGGTCATGATTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-013 | 22 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGCAGCGTAAGTCTGGTTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-014 | 23 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTATTActTgagt |
| | | AAGACGATTAATGGTTCTGGTTCTCCGCATTCTAAGGCGCAGGCGCGTAAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-015 | 24 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGTATATTGTGGGTTCTGGTTCTCCGCATTCTAAGGCGCAGAATCAGCAGACGUT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-016 | 25 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATOTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTTCTAAGCGTGGTTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-017 | 26 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGGGTCTGGGTGGTTCTGGTTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-018 | 27 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTCATGATTCTCCGCATTCTAAGGCGCAGAATCTGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-019 | 28 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGGTGAATGGTCATGATTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGGTAttTAACgaGgAActTa |
| TTM-020 | 29 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCETTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActlgagt |
| | | AAGACGATTAATGGTCATGATTCTCCGCATTCTAAGGCGCTGAATCAGCAGACGUT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGGTAttTAACgaGgAActTa |
| TTM-021 | 30 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTCATGATTCTCCGCATTCTAAGGCGCAGAATCAGCAGTCTET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-022 | 31 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTCATGATTCTCCGCATTCTAAGGCGCAGAATCAGCAGATTET |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-023 | 32 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAGTAtcTGTAtTActTgagt |
| | | AAGACGATTAATGGTTCTGGTTCTCCGCATTTTACGCGTCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-024 | 33 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGGGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGTCTAATGGTCATGATTCTCCGCATTCTAAGGCGCAGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-025 | 34 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTTCTGGTTCTCCGCATTCTCTGCCGTGGAATCAGCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-026 | 35 | ATGGCTGCCGATGGTTATCTTCCAGattggcTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGAtCAgTAtcTGTAtTActTgagt |
| | | AAGACGATTAATGGTCATGATTCTCCGCATTCTAAGGCGCAGAATCATCAGACGtT |
| | | gAAgTTttcgGTaGCtGGtCCtAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGtggccacaaaccaccagagtGCCCAAGCACAGGCGC |
| | | AGaccggctgggttcaaaaccaAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACgcGgTAttTAACgaGgAActTa |
| TTM-027 | 5 | ATGGCTGCCGATGGTTATCTTCCAGATTGGCTCGAGGACAACCTTAGTGAAGGAAT |
| | | TCGCGAGTGGTGGGCTTTGAAACCTGGAGCCCCTCAACCCAAGGCAAATCAACAAC |
| | | ATCAAGACAACGCTCGAGGTCTTGTGCTTCCGGGTTACAAATACCTTGGACCCGGC |
| | | AACGGACTCGACAAGGGGGAGCCGGTCAACGCAGCAGACGCGGCGGCCCTCGAGCA |
| | | CGACAAGGCCTACGACCAGCAGCTCAAGGCCGGAGACAACCCGTACCTCAAGTACA |
| | | ACCACGCCGACGCCGAGTTCCAGGAGCGGCTCAAAGAAGATACGTCTTTTGGGGGC |
| | | AACCTCGGGCGAGCAGTCTTCCAGGCCAAAAAGAGGCTTCTTGAACCTCTTGGTCT |
| | | GGTTGAGGAAGCGGCTAAGACGGCTCCTGGAAAGAAGAGGCCTGTAGAGCAGTCTC |
| | | CTCAGGAACCGGACTCCTCCGCGGGTATTGGCAAATCGGGTGCACAGCCCGCTAAA |
| | | AAGAGACTCAATTTCGGTCAGACTGGCGACACAGAGTCAGTCCCAGACCCTCAACC |
| | | AATCGGAGAACCTCCCGCAGCCCCCTCAGGTGTGGGATCTCTTACAATGGCTTCAG |
| | | GTGGTGGCGCACCAGTGGCAGACAATAACGAAGGTGCCGATGGAGTGGGTAGTTCC |
| | | TCGGGAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATCTCCA |
| | | ACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCA |
| | | GCGACTCATCAACAACAACTGGGGATTCCGGCCTAAGCGACTCAACTTCAAGCTCT |
| | | TCAACATTCAGGTCAAAGAGGTTACGGACAACAATGGAGTCAAGACCATCGCCAAT |
| | | AACCTTACCAGCACGGTCCAGGTCTTCACGGACTCAGACTATCAGCTCCCGTACGT |
| | | GCTCGGGTCGGCTCACGAGGGCTGCCTCCCGCCGTTCCCAGCGGACGTTTTCATGA |
| | | TTCCTCAGTACGGGTATCTGACGCTTAATGATGGAAGCCAGGCCGTGGGTCGTTCG |
| | | TCCTTTTACTGCCTGGAATATTTCCCGTCGCAAATGCTAAGAACGGGTAACAACTT |
| | | CCAGTTCAGCTACGAGTTTGAGAACGTACCTTTCCATAGCAGCTACGCTCACAGCC |
| | | AAAGCCTGGACCGACTAATGAATCCACTCATCGACCAATACTTGTACTATCTCTCT |
| | | AAGACTGAGAATGTGAGCGGGAGCCCTCATAGCAAGGCTCAGAATCAGCAGACTCT |
| | | AAAATTCAGTGTGGCCGGACCCAGCAACATGGCTGTCCAGGGAAGAAACTACATAC |
| | | CTGGACCCAGCTACCGACAACAACGTGTCTCAACCACTGTGACTCAAAACAACAAC |
| | | AGCGAATTTGCTTGGCCTGGAGCTTCTTCTTGGGCTCTCAATGGACGTAATAGCTT |
| | | GATGAATCCTGGACCTGCTATGGCCAGCCACAAAGAAGGAGAGGACCGTTTCTTTC |
| | | CTTTGTCTGGATCTTTAATTTTTGGCAAACAAGGAACTGGAAGAGACAACGTGGAT |
| | | GCGGACAAAGTCATGATAACCAACGAAGAAGAAATTAAAACTACTAACCCGGTAGC |
| | | AACGGAGTCCTATGGACAAGTGGCCACAAACCACCAGAGTGCCCAAGCACAGGCGC |
| | | AGACCGGCTGGGTTCAAAACCAAGGAATACTTCCGGGTATGGTTTGGCAGGACAGA |
| | | GATGTGTACCTGCAAGGACCCATTTGGGCCAAAATTCCTCACACGGACGGCAACTT |
| | | TCACCCTTCTCCGCTGATGGGAGGGTTTGGAATGAAGCACCCGCCTCCTCAGATCC |
| | | TCATCAAAAACACACCTGTACCTGCGGATCCTCCAACGGCCTTCAACAAGGACAAG |
| | | CTGAACTCTTTCATCACCCAGTATTCTACTGGCCAAGTCAGCGTGGAGATCGAGTG |
| | | GGAGCTGCAGAAGGAAAACAGCAAGCGCTGGAACCCGGAGATCCAGTACACTTCCA |
| | | ACTATTACAAGTCTAATAATGTTGAATTTGCTGTTAATACTGAAGGTGTATATAGT |
| | | GAACCCCGCCCCATTGGCACCAGATACCTGACTCGTAATCTG |

In some embodiments, the polynucleotide encoding an AAV capsid variant comprises the nucleotide sequence of SEQ ID NO: 983 or 984, or a nucleotide sequence with at least 70% (e.g., at least 70%, at least 75%, at least 80%, at least 85%, at least 90%, at least 95%, at least 96%, at least 97%, at least 98%, or at least 99%) sequence identity thereto.

In some embodiments, the polynucleotide encoding an AAV capsid variant, comprises the nucleotide sequence of any one of SEQ ID NOs: 5, 12-35, or a nucleotide sequence with at least 70% (e.g., at least 70%, at least 75%, at least 80%, at least 85%, at least 90%, at least 95%, at least 96%, at least 97%, at least 98%, or at least 99%) sequence identity thereto.

In some embodiments, the polynucleotide encoding an AAV capsid variant comprises the nucleotide sequence of SEQ ID NO: 12 or a nucleotide sequence with at least 70% (e.g., at least 70%, at least 75%, at least 80%, at least 85%, at least 90%, at least 95%, at least 96%, at least 97%, at least 98%, or at least 99%) sequence identity thereto.

In some embodiments, the polynucleotide encoding an AAV capsid variant comprises the nucleotide sequence of SEQ ID NO: 5 or a nucleotide sequence with at least 70% (e.g., at least 70%, at least 75%, at least 80%, at least 85%, at least 90%, at least 95%, at least 96%, at least 97%, at least 98%, or at least 99%) sequence identity thereto.

In some embodiments, the polynucleotide encoding an AAV capsid variant comprises the nucleotide sequence of SEQ ID NO: 983, or a nucleotide sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, the nucleotide sequence encoding an AAV capsid variant described herein, comprises a nucleotide sequence comprising at least one, at least two, or at least three modifications but not more than 30, not more than 20, or not more than 10 modifications, relative to the nucleotide sequence of SEQ ID NO: 983. In some embodiments, the nucleotide sequence encoding an AAV capsid variant described herein, comprises a nucleotide sequence comprising at least one, at least two, or at least three substitutions, but not more than 30, not more than 20, or not more than 10 substitutions relative to the amino acid sequence of SEQ ID NO: 983. In some embodiments, the nucleic acid sequence encoding an AAV capsid variant is codon optimized. In some embodiments, the polynucleotide encoding an AAV capsid variant comprises SEQ ID NO: 983. In some embodiments, the polynucleotide encoding an AAV capsid variant comprises SEQ ID NO: 983. In some embodiments, the polynucleotide encoding an AAV capsid variant consists of SEQ ID NO: 983.

In some embodiments, the polynucleotide encoding an AAV capsid variant comprises the nucleotide sequence of SEQ ID NO: 984, or a nucleotide sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, the nucleotide sequence encoding an AAV capsid variant described herein, comprises a nucleotide sequence comprising at least one, at least two, or at least three modifications, but not more than 30, not more than 20, or not more than 10 modifications, relative to the nucleotide sequence of SEQ ID NO: 984. In some embodiments, the nucleotide sequence encoding an AAV capsid variant described herein, comprises a nucleotide sequence comprising at least one, at least two, or at least three modifications, but not more than 30, not more than 20, or not more than 10 different nucleotides, relative to the amino acid sequence of SEQ ID NO: 984. In some embodiments, the nucleic acid sequence encoding an AAV capsid variant is codon optimized. In some embodiments, the polynucleotide encoding an AAV capsid variant comprises SEQ ID NO: 984. In some embodiments, the polynucleotide encoding an AAV capsid variant consists of SEQ ID NO: 984.

In some embodiments, an AAV capsid variant comprises the amino acid sequence of any one of SEQ ID NOs: 4, 36-59, 981, or 982, or an amino acid sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, an AAV capsid variant comprises an amino acid sequence comprising at least one, at least two, or at least three modifications, but not more than 30, not more than 20, or not more than 10 modifications, relative to the amino acid sequence of SEQ ID NO: 4, 36-59, 981, or 982. In some embodiments, the AAV capsid variant, comprises an amino acid sequence comprising at least one, at least two, or at least three, but not more than 30, not more than 20, or not more than 10 different amino acids, relative to the amino acid sequence of SEQ ID NO: 4, 36-59, 981, or 982.

In some embodiments, the AAV capsid variant comprises one or more substitutions in loop IV and comprises the amino acid sequence of SEQ ID NO: 981, or an amino acid sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, an AAV capsid variant comprises an amino acid sequence comprising at least one, at least two, or at least three modifications, but not more than 30, not more than 20, or not more than 10 modifications, relative to the amino acid sequence of SEQ ID NO: 981. In some embodiments, an AAV capsid variant comprises an amino acid sequence comprising at least one, at least two, or at least three substitutions, but not more than 30, not more than 20, or not more than 10 substitutions, relative to the amino acid sequence of SEQ ID NO: 981.

In some embodiments, an AAV capsid variant comprises the amino acid sequence of SEQ ID NO: 982, or an amino acid sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, an AAV capsid variant comprises an amino acid sequence comprising at least one, at least two, or at least three modifications, but not more than 30, not more than 20, or not more than 10 modifications, relative to the amino acid sequence of SEQ ID NO: 982. In some embodiments, the AAV capsid variant, comprises an amino acid sequence comprising at least one, at least two, or at least three, but not more than 30, not more than 20, or not more than 10 different amino acids, relative to the amino acid sequence of SEQ ID NO: 982. In some embodiments, an AAV capsid variant comprises an amino acid sequence that is at least 97% identical to SEQ ID NO: 982. In some embodiments, an AAV capsid variant comprises an amino acid sequence that is at least 98% identical to SEQ ID NO: 982. In some embodiments, an AAV capsid variant comprises an amino acid sequence that is at least 99% identical to SEQ ID NO: 982. In some embodiments, an AAV capsid variant comprises the amino acid sequence of SEQ ID NO: 982. In some embodiments, an AAV capsid variant consists of the amino acid sequence of SEQ ID NO: 982. In some embodiments, an AAV capsid variant comprises amino acid residues 2-742 of SEQ ID NO: 982. In some embodiments, an AAV capsid variant consists of amino acid residues of 2-742 of SEQ ID NO: 982.

In some embodiments, an AAV capsid variant comprises the amino acid sequence of SEQ ID NO: 36, or an amino acid sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, an AAV capsid variant comprises an amino acid sequence comprising at least one, at least two, or at least three modifications, but not more than 30, not more than 20, or not more than 10 modifications, relative to the amino acid sequence of SEQ ID NO: 36. In some embodiments, the AAV capsid variant, comprises an amino acid sequence comprising at least one, at least two, or at least three, but not more than 30, not more than 20, or not more than 10 different amino acids, relative to the amino acid sequence of SEQ ID NO: 36. In some embodiments, an AAV capsid variant comprises an amino acid sequence that is at least 97% identical to SEQ ID NO: 36. In some embodiments, an AAV capsid variant comprises an amino acid sequence that is at least 98% identical to SEQ ID NO: 36. In some embodiments, an AAV capsid variant comprises an amino acid sequence that is at least 99% identical to SEQ ID NO: 36. In some embodiments, an AAV capsid variant comprises the amino acid sequence of SEQ ID NO: 36. In some embodiments, an AAV capsid variant consists of the amino acid sequence of SEQ ID NO: 36. In some embodiments, an AAV capsid variant comprises amino acid residues 2-742 of SEQ ID NO: 36. In some embodiments, an AAV capsid variant consists of amino acid residues of 2-742 of SEQ ID NO: 36.

In some embodiments, an AAV capsid variant comprises the amino acid sequence of SEQ ID NO: 4, or an amino acid sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, an AAV capsid variant comprises an amino acid sequence comprising at least one, at least two, or at least three modifications, but not more than 30, not more than 20, or not more than 10 modifications, relative to the amino acid sequence of SEQ ID NO: 4. In some embodiments, the AAV capsid variant, comprises an amino acid sequence comprising at least one, at least two, or at least three, but not more than 30, not more than 20, or not more than 10 different amino acids, relative to the amino acid sequence of SEQ ID NO: 4. In some embodiments, an AAV capsid variant comprises an amino acid sequence that is at least 97% identical to SEQ ID NO: 4. In some embodiments, an AAV capsid variant comprises an amino acid sequence that is at least 98% identical to SEQ ID NO: 4. In some embodiments, an AAV capsid variant comprises an amino acid sequence that is at least 99% identical to SEQ ID NO: 4. In some embodiments, an AAV capsid variant comprises the amino acid sequence of SEQ ID NO: 4. In some embodiments, an AAV capsid variant consists of the amino acid sequence of SEQ ID NO: 4. In some embodiments, an AAV capsid variant comprises amino acid residues 2-742 of SEQ ID NO: 4. In some embodiments, an AAV capsid variant consists of amino acid residues of 2-742 of SEQ ID NO: 4.

In some embodiments, an AAV capsid variant comprises the amino acid sequence of SEQ ID NO: 39, or an amino acid sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, an AAV capsid variant comprises an amino acid sequence comprising at least one, at least two, or at least three modifications, but not more than 30, not more than 20, or not more than 10 modifications, relative to the amino acid sequence of SEQ ID NO: 39. In some embodiments, the AAV capsid variant, comprises an amino acid sequence comprising at least one, at least two, or at least three, but not more than 30, not more than 20, or not more than 10 different amino acids, relative to the amino acid sequence of SEQ ID NO: 39. In some embodiments, an AAV capsid variant comprises an amino acid sequence that is at least 97% identical to SEQ ID NO: 39. In some embodiments, an AAV capsid variant comprises an amino acid sequence that is at least 98% identical to SEQ ID NO: 39. In some embodiments, an AAV capsid variant comprises an amino acid sequence that is at least 99% identical to SEQ ID NO: 39. In some embodiments, an AAV capsid variant comprises the amino acid sequence of SEQ ID NO: 39. In some embodiments, an AAV capsid variant consists of the amino acid sequence of SEQ ID NO: 39. In some embodiments, an AAV capsid variant comprises amino acid residues 2-742 of SEQ ID NO: 39. In some embodiments, an AAV capsid variant consists of amino acid residues of 2-742 of SEQ ID NO: 39.

In some embodiments, an AAV capsid variant comprises the amino acid sequence of SEQ ID NO: 51, or an amino acid sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, an AAV capsid variant comprises an amino acid sequence comprising at least one, at least two, or at least three modifications, but not more than 30, not more than 20, or not more than 10 modifications, relative to the amino acid sequence of SEQ ID NO: 51. In some embodiments, the AAV capsid variant, comprises an amino acid sequence comprising at least one, at least two, or at least three, but not more than 30, not more than 20, or not more than 10 different amino acids, relative to the amino acid sequence of SEQ ID NO: 51. In some embodiments, an AAV capsid variant comprises an amino acid sequence that is at least 97% identical to SEQ ID NO: 51. In some embodiments, an AAV capsid variant comprises an amino acid sequence that is at least 98% identical to SEQ ID NO: 51. In some embodiments, an AAV capsid variant comprises an amino acid sequence that is at least 99% identical to SEQ ID NO: 51. In some embodiments, an AAV capsid variant comprises the amino acid sequence of SEQ ID NO: 51. In some embodiments, an AAV capsid variant consists of the amino acid sequence of SEQ ID NO: 51. In some embodiments, an AAV capsid variant comprises amino acid residues 2-742 of SEQ ID NO: 51. In some embodiments, an AAV capsid variant consists of amino acid residues of 2-742 of SEQ ID NO: 51.

In some embodiments, an AAV capsid variant comprises the amino acid sequence of SEQ ID NO: 52, or an amino acid sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, an AAV capsid variant comprises an amino acid sequence comprising at least one, at least two, or at least three modifications, but not more than 30, not more than 20, or not more than 10 modifications, relative to the amino acid sequence of SEQ ID NO: 52. In some embodiments, the AAV capsid variant, comprises an amino acid sequence comprising at least one, at least two, or at least three, but not more than 30, not more than 20, or not more than 10 different amino acids, relative to the amino acid sequence of SEQ ID NO: 52. In some embodiments, an AAV capsid variant comprises an amino acid sequence that is at least 97% identical to SEQ ID NO: 52. In some embodiments, an AAV capsid variant comprises an amino acid sequence that is at least 98% identical to SEQ ID NO: 52. In some embodiments, an AAV capsid variant comprises an amino acid sequence that is at least 99% identical to SEQ ID NO: 52. In some embodiments, an AAV capsid variant comprises the amino acid sequence of SEQ ID NO: 52. In some embodiments, an AAV capsid variant consists of the amino acid sequence of SEQ ID NO: 52. In some embodiments, an AAV capsid variant comprises amino acid residues 2-742 of SEQ ID NO: 52. In some embodiments, an AAV capsid variant consists of amino acid residues of 2-742 of SEQ ID NO: 52.

In some embodiments, an AAV capsid variant comprises an amino acid sequence encoded by the nucleotide sequence of SEQ ID NO: 983 or 984, or a nucleotide sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, an AAV capsid variant comprises an amino acid sequence encoded by a nucleotide sequence comprising at least one, at least two, or at least three substitutions, but not more than 30, not more than 20, or not more than 10 substitutions, relative to the amino acid sequence of SEQ ID NO: 983. In some embodiments, an AAV capsid variant comprises an amino acid sequence encoded by a nucleotide sequence comprising at least one, at least two, or at least three modifications, but not more than 30, not more than 20, or not more than 10 modifications, relative to the nucleotide sequence of SEQ ID NO: 983.

In some embodiments, an AAV capsid variant comprises an amino acid sequence encoded by the nucleotide sequence of any one of SEQ ID NOs: 5 or 12-35, or a nucleotide sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, an AAV capsid variant comprises an amino acid sequence encoded by a nucleotide sequence comprising at least one, at least two, or at least three, but not more than 30, not more than 20, or not more than 10 different nucleotides, relative to the amino acid sequence of any one of SEQ ID NOs: 5 or 12-35. In some embodiments, an AAV capsid variant comprises an amino acid sequence encoded by a nucleotide sequence comprising at least one, at least two, or at least three modifications, but not more than 30, not more than 20, or not more than 10 modifications, relative to the nucleotide sequence of any one of SEQ ID NOs: 5 or 12-35.

In some embodiments, an AAV capsid variant comprises a VP1, VP2, VP3 protein, wherein at least one of the VP1, VP2, and VP3 comprise one or more insertions in loop IV. In some embodiments, an AAV capsid variant comprises the amino acid sequence corresponding to amino acids 138-742, e.g., a VP2, of SEQ ID NO: 981 or 982, or a sequence with at least 70% (e.g., at least 70%, at least 75%, at least 80%, at least 85%, at least 90%, at least 95%, at least96%, at least 97%, at least 98%, or at least 99%) sequence identity thereto. In some embodiments, the AAV capsid protein comprises the amino acid sequence corresponding to amino acids 203-742, e.g., a VP3, of SEQ ID NO: 981 or 982, or a sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, the AAV capsid variant comprises the amino acid sequence corresponding to amino acids 1-742, e.g., a VP1, of SEQ ID NO: 981 or 982, or an amino acid sequence with at least 70% (e.g., at least 70%, at least 75%, at least 80%, at least 85%, at least 90%, at least 95%, at least 96%, at least 97%, at least 98%, or at least 99%) sequence identity thereto.

In some embodiments, an AAV capsid variant comprises the amino acid sequence corresponding to amino acids 138-742, e.g., a VP2, of SEQ ID NO: 982, or a sequence with at least 70% (e.g., at least 70%, at least 75%, at least 80%, at least 85%, at least 90%, at least 95%, at least 96%, at least 97%, at least 98%, or at least 99%, e.g., 100%) sequence identity thereto. In some embodiments, the AAV capsid protein comprises the amino acid sequence corresponding to amino acids 203-742, e.g., a VP3, of SEQ ID NO: 982, or a sequence with at least 70% (e.g., at least 70%, at least 75%, at least 80%, at least 85%, at least 90%, at least 95%, at least 96%, at least 97%, at least 98%, or at least 99%, e.g., 100%) sequence identity thereto. In some embodiments, the AAV capsid variant comprises the amino acid sequence corresponding to amino acids 1-742, e.g., a VP1, of SEQ ID NO: 982, or an amino acid sequence with at least 70% (e.g., at least 70%, at least 75%, at least 80%, at least 85%, at least 90%, at least 95%, at least 996%, at least 97%, at least 98%, or at least 99%, e.g., 100%) sequence identity thereto. In some embodiments, the AAV capsid variant comprises or consists of the amino acid sequence of SEQ ID NO: 982. In some embodiments, the AAV capsid variant comprises or consists of amino acid residues 2-742 of SEQ ID NO: 982.

In some embodiments, an AAV capsid variant comprises the amino acid sequence corresponding to amino acids 138-742, e.g., a VP2, of SEQ ID NO: 36, or a sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, the AAV capsid protein comprises the amino acid sequence corresponding to amino acids 203-742, e.g., a VP3, of SEQ ID NO: 36, or a sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, the AAV capsid variant comprises the amino acid sequence corresponding to amino acids 1-742, e.g., a VP1, of SEQ ID NO: 36, or an amino acid sequence with at least 70% (e.g., at least 70%, at least 75%, at least 80%, at least 85%, at least 90%, at least 95%, at least 96%, at least 97%, at least 98%, or at least 99% sequence identity thereto. In some embodiments, the AAV capsid variant comprises or consists of the amino acid sequence of SEQ ID NO: 36. In some embodiments, the AAV capsid variant comprises or consists of amino acid residues 2-742 of SEQ ID NO: 36.

In some embodiments, an AAV capsid variant comprises the amino acid sequence corresponding to amino acids 138-742, e.g., a VP2, of SEQ ID NO: 4, or a sequence with at least 70% (e.g., at least 70%, at least 75%, at least 800, at least 85%, at least 90%, at least 95%, at least 96%, at least 97%, at least 98%, or at least 99%) sequence identity thereto. In some embodiments, the AAV capsid protein comprises the amino acid sequence corresponding to amino acids 203-742, e.g., a VP3, of SEQ ID NO: 4, or a sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, the AAV capsid variant comprises the amino acid sequence corresponding to amino acids 1-742, e.g., a VP1, of SEQ ID NO: 4, or an amino acid sequence with at least 70% (e.g., at least 70%, at least 75%, at least 80%, at least 85%, at least 90%, at least 95%, at least 96%, at least 97%, at least 98%, or at least 99% sequence identity thereto. In some embodiments, the AAV capsid variant comprises or consists of the amino acid sequence of SEQ ID NO: 4. In some embodiments, the AAV capsid variant comprises or consists of amino acid residues 2-742 of SEQ ID NO: 4.

In some embodiments, an AAV capsid variant comprises a VP1, VP2, VP3 protein, or a combination thereof. In some embodiments, an AAV capsid variant comprises the amino acid sequence corresponding to amino acids 138-742, e.g., a VP2, of any one of SEQ ID NOs: 4, 36-59, or a sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, the AAV capsid protein comprises the amino acid sequence corresponding to amino acids 203-742, e.g., a VP3, of any one of SEQ ID NOs: 4.36-59, or a sequence with at least 70% (e.g., at least 70%, at least 75%, at least 80%, at least 85%, at least 90%, at least 95%, at least 96%, at least 97%, at least 98%, or at least 99%) sequence identity thereto. In some embodiments, the AAV capsid variant comprises the amino acid sequence corresponding to amino acids 1-742, e.g., a VP1, of any one of SEQ ID NOs: 4, 36-59, or an amino acid sequence with at least 70% (e.g., at least 70%, at least 75%, at least 80%, at least 85%, at least 90%, at least 95%, at least 96%, at least 97%, at least 98%, or at least 99%) sequence identity thereto.

In some embodiments, an AAV capsid variant has an increased tropism for a CNS cell or tissue, e.g., a brain cell, brain tissue, spinal coni cell, or spinal cord tissue, relative to the tropism of an AAV capsid comprising SEQ ID NO: 138.

In some embodiments, an AAV capsid variant transduces a brain region, e.g., a midbrain region (e.g., the hippocampus, or thalamus) or the brain stem. In some embodiments, the level of transduction is at least 5, at least 10, at least 15, at least 20, at least 25, at least 30, at least 35, at least 40, at least 45, at least 50, at least 55, at least 60, or at least 65-fold greater as compared to an AAV capsid comprising SEQ ID NO: 138. In some embodiments, the level of transduction is at least 30, at least 35, at least 40, at least 45, at least 50, at least 55, at least 60, or at least 65-fold greater as compared to an AAV capsid comprising SEQ ID NO: 138.

In some embodiments, an AAV capsid variant is enriched at least 3, at least 4, at least 5, at least 6, at least 7, at least 8, at least 9, or at least 10-fold in the brain compared to an AAV capsid comprising of SEQ ID NO: 138. In some embodiments, an AAV capsid variant is enriched at least 20, at least 25, at least 30, at least 35, at least 40, at least 45, at least 50, at least 55, at least 60, at least 65, at least 70, at least 75, at least 80 or at least 85-fold in the brain compared to an AAV capsid comprising SEQ ID NO: 138.

In some embodiments, an AAV capsid variant is enriched in the brain of at least two to three species, e.g., a non-human primate and rodent (e.g., mouse) species, compared to an AAV capsid comprising SEQ ID NO: 138. In some embodiments, an AAV capsid variant is enriched at least 2, at least 3, at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 115, at least 120, at least 125, at least 130, at least 135, at least 140, at least 145, at least 150, at least 155, at least 160, at least 165, at least 170, at least 175, at least 180, at least 190, at least 200, at least 205, or at least 210-fold in the brain of at least two to at least three species, e.g., a non-human primate and rodent (e.g., mouse) species, compared to an AAV capsid comprising SEQ ID NO: 138. In some embodiments, the at least two to at least three species are Macaca fascicularis, Chlorocebus sabaeus, Callithrix jacchus, and/or mouse (e.g., BALB/c mice, C57BL/6 mice, and/or CD-1 outbred mice).

In some embodiments, an AAV capsid variant is enriched at least 2, at least 2.5, at least 3, at least 3.5, at least 4, at least 4.5, at least 5, at least 5.5, at least 6, at least 6.5, at least 7, at least 7.5, or at least 8-fold, in the brain compared to an AAV capsid variant comprising SEQ ID NO: 981. In some embodiments, an AAV capsid variant is enriched about 2, 2.5, 3, 3.5, 4, 4.5, 5, or 5.5-fold, in the brain compared to an AAV capsid variant comprising SEQ ID NO: 982.

In some embodiments, an AAV capsid variant delivers an increased level of viral genomes to a brain region. In some embodiments, the level of viral genomes is increased by at least 20, at least 25, at least 30, at least 35, at least 40, at least 45, or at least 50-fold, as compared to an AAV capsid comprising SEQ ID NO: 138. In some embodiments, the brain region comprises a midbrain region (e.g., the hippocampus or thalamus) and/or the brainstem.

In some embodiments, an AAV capsid variant delivers an increased level of STXBP1 to a brain region. In some embodiments, the level of STXBP1 is increased by at least 20, at least 25, at least 30, at least 35, at least 40, at least 45, at least 50, at least 55, at least 60, at least 65, or at least 70-fold, as compared to an AAV capsid comprising SEQ ID NO: 138. In some embodiments, the brain region comprises a midbrain region (e.g., the hippocampus or thalamus) and/or the brainstem.

In some embodiments, an AAV capsid variant is enriched at least 5, at least 10, at least 15, at least 20, at least 25, at least 30, or at least 35-fold, in the spinal cord compared to an AAV capsid comprising SEQ ID NO: 138.

In some embodiments, an AAV capsid variant shows preferential transduction in a brain region relative to the transduction in the dorsal root ganglia (DRG). In some embodiments, the AAV capsid variant shows preferential transduction in a brain region relative to the transduction in the liver. In some embodiments, the AAV capsid variant shows preferential transduction in a brain region relative to the transduction in the liver and the DRG. In some embodiments, the AAV capsid variant shows preferential transduction in a brain region relative to the transduction in the heart. In some embodiments, the AAV capsid variant shows preferential transduction in a brain region relative to the transduction in the heart and DRG. In some embodiments, the AAV capsid variant shows preferential transduction in a brain region relative to the transduction in the heart. DRG, and liver. In sonic embodiments, the AAV capsid variant shows preferential transduction in a brain region and/or a heart region relative to the transduction in the liver and DRG.

In some embodiments, an AAV capsid variant is capable of transducing non-neuronal cells, e.g., glial cells (e.g., oligodendrocytes or astrocytes). In some embodiments, the AAV capsid variant is capable of transducing neuronal cells and non-neuronal cells, e.g., glial cells (e.g., oligodendrocytes or astrocytes). In some embodiments, the non-neuronal cells are glial cells, oligodendrocytes (e.g., Olig2 positive oligodendrocytes), or astrocytes (e.g., Olig2 positive astrocytes). In some embodiments, the AAV capsid variant is capable of transducing Olig2 positive cells, e.g., Olig2 positive astrocytes or Olig2 positive oligodendrocytes.

In some embodiments, an AAV capsid variant of the present disclosure has decreased tropism for the liver. In some embodiments, an AAV capsid variant comprises a modification that results in reduced tropism (e.g., de-targeting) and/or activity in the liver. In some embodiments, the reduced tropism in the liver is compared to an otherwise similar capsid that does not comprise the modification, e.g., a wild-type capsid polypeptide. In some embodiments, an AAV capsid variant comprises a modification that results in one or more of the following properties: (1) reduced tropism in the liver; (2) reduced, e.g., de-targeted expression in the liver; (3) reduced activity in the liver, and/or (4) reduced binding to galactose. In some embodiments, the reduction in any one or all of properties (1)-(3) is compared to an otherwise similar AAV capsid variant that does not comprise the modification.

Exemplary modifications are provided in WO 2018/119330: Pulicherla et al. (2011) Mol. Ther. 19(6): 1070-1078; Adachi et al. (2014) Nature Communications 5(3075), DOI: 10.1038/ncomms4075; and Bell et al. (2012) J. Virol. 86(13): 7326-33; the contents of which are hereby incorporated by reference in their entirety. In some embodiments, the AAV capsid variant comprises a modification at amino acid N470 (e.g., N470A), D271 (e.g., D271A), N272 (e.g., N272A), Y446 (e.g., Y446A), N498 (e.g., N498Y or N498I), W503 (e.g., W503R or W503A), L620 (e.g., L620F), or a combination thereof, as numbered according to SEQ ID NO: 138. In some embodiments, the AAV capsid variant comprises one, two, three, four, five, or all of an amino acid other than N at amino acid 470 (e.g., A), an amino acid other than D at amino acid 271 (e.g., A), an amino acid other than N at amino acid 272 (e.g., A), an amino acid other than Y at amino acid 446 (e.g., A), and amino acid other than N at amino acid 498 (e.g., Y or I), and amino acid other than W at amino acid 503 (e.g., R or A), and amino acid other than L at amino acid 620 (e.g., F), as numbered according to SEQ ID NO: 138. In some embodiments, the AAV capsid variant comprises a modification at amino acid N470 (e.g., N470A), D271 (e.g., D271A), N272 (e.g., N272A), Y446 (e.g., Y446A), and W503 (e.g., W503R or W503A), numbered according to SEQ ID NO: 138. In some embodiments, the AAV capsid variant comprises a modification at N498 (e.g., N498Y) and L620 (e.g., L620F).

In some embodiments, the AAV capsid variant comprises a modification as described in Adachi et al. (2014) Nature Communications 5(3075), DOI: 10.1038/ncomms4075, the contents of which are hereby incorporated by reference in their entirety. Exemplary modifications that alter or do not alter tissue transduction in at least the brain, liver, heart, lung, and/or kidney can be found in Supplementary Data 2 showing the AAV Barcode-Seq data obtained with AAV9-AA-VBCLib of Adachi et al. (supra), the contents of which are hereby incorporated by reference in their entirety.

In some embodiments, the AAV capsid variant is an isolated capsid variant. In some embodiments, the AAV capsid variant is a recombinant capsid variant. In some embodiments, a polynucleotide encoding an AAV capsid polypeptide, e.g., an AAV capsid variant is an isolated and/or a recombinant AAV capsid polypeptide.

Also provided herein are polynucleotide sequences encoding any of the AAV capsid variants described above and AAV particles, vectors, and cells comprising the same.

### Certain Properties of AAV Capsids

In some embodiments, an AAV particle of the present disclosure may comprise a capsid protein or variant thereof any natural or recombinant AAV serotype. AAV serotypes may differ in characteristics such as, but not limited to, packaging, tropism, transduction, and immunogenic profiles.

In some embodiments, an AAV capsid variant described herein allows for blood brain barrier penetration following intravenous administration. In some embodiments, the AAV capsid variant allows for blood brain barrier penetration following intravenous administration, focused ultrasound (FUS), e.g., coupled with the intravenous administration of microbubbles (FUS-MB), or MRI-guided FUS coupled with intravenous administration. In some embodiments the AAV capsid variant allows for increased distribution to a brain region. In some embodiments, the brain region comprises a frontal cortex, sensory cortex, motor cortex, caudate, dentate nucleus, cerebellar cortex, cerebral cortex, brain stem, hippocampus, thalamus, putamen, or a combination thereof. In some embodiments, the AAV capsid variant allows for preferential transduction in a brain region relative to the transduction in the dorsal root ganglia (DRG). In some embodiments, the AAV capsid variant allows for preferential transduction in a brain region relative to the transduction in the liver. In some embodiments, the AAV capsid variant allows for transduction in neuronal cells. In some embodiments, the AAV capsid variant allows for preferential transduction to GABAergic neurons and/or glutamatergic neurons relative to other cells. In some embodiments, the AAV capsid variant allows for transduction in a non-neuronal cell, e.g., a glial cell (e.g., an astrocyte, an oligodendrocyte, or a combination thereof).

In some embodiments, an AAV capsid variant allows for increased distribution to a spinal cord region. In some embodiments, the spinal region comprises a cervical spinal cord region, thoracic spinal cord region, and/or lumbar spinal cord region.

In some embodiments, the initiation codon for translation of the AAV VP1 capsid protein. e.g., a capsid variant, described herein may be CTG, TTG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (e.g., capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptidc synthesis by protein processing enzymes such as Met-aminopeptidases. This “MetAA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267: Hwang, et al. N-Terminal Acetylation of Cellular Proteins Creates Specific Degradation Signals. Science. 2010 Feb. 19, 327(5968): 973-977; the contents of which are each incorporated herein by reference in its entirety.

According to the present disclosure, references to capsid proteins, e.g., AAV capsid variants, is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce, or result in capsid proteins of the present disclosure. A direct reference to a capsid protein or capsid polypeptide (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met 1” amino acid (Met−) of the 736 amino acid Met+ sequence. As a second non-limiting example, reference to a VP1 poly peptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA 1+), corresponding VP capsid proteins which lack the Met1/AA 1 amino acid as a result of Met/AA1-clipping (Met−/AA 1−), and combinations thereof (Met+/AA1+ and Met-/AA1−).

As a non-limiting example, an AAV capsid serotype can include VP1 (Met+/AA1+). VP1 (Met−/AA1−), or a combination of VP1 (Met+/AA1+) and VP1 (Met−/AA1−), An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Additional AAV Sequences

In some embodiments, an AAV capsid polypeptide or AAV capsid variant described herein may comprise a VOY101 capsid polypeptide, an AAVPHP.B (PHP.B) capsid polypeptide, a AAVPHP.N (PHP.N) capsid polypeptide, an AAV1 capsid polypeptide, an AAV2 capsid polypeptide, an AAV5 capsid poly peptide, an AAV9 capsid polypeptide, an AAV9 K449R capsid polypeptide, an AAVrh10 capsid polypeptide, or a functional variant thereof. In some embodiments, the AAV capsid polypeptide, e.g., AAV capsid variant, comprises an amino acid sequence of any of the AAV capsid polypeptides in Table 6, or an amino acid sequence substantially identical (e.g., having at least 95%, at least 96%, at least 97%, at least 98%, or at least 99% sequence identity) thereto. In some embodiments, the nucleotide sequence encoding the AAV capsid polypeptide comprises any one of the nucleotide sequences in Table 6, or a nucleotide sequence substantially identical (e.g., having at least 90%, at least 92%, at least 95%, at least 97%, at least 98%, or at least 99% sequence identity) thereto.

In some embodiments, an AAV capsid polypeptide or an AAV capsid variant described herein comprises an amino acid sequence having at least 95%, at least 96%, at least 97%, at least 98%, or at least 99% sequence identity) to SEQ ID NO: 138. In some embodiments the AAV capsid polypeptide or the AAV capsid variant comprises an amino acid sequence comprising at least one, at least two, or at least three modifications, but no more than 30, no more than 20, or no more than 10 modifications, relative to the amino acid sequence of SEQ ID NO: 138. In some embodiments, the AAV capsid polypeptide or the AAV capsid variant, comprises an amino acid sequence encoded by a nucleotide sequence having at least 90%, at least 92%, at least 95%, at least 97%, at least 98%, or at least 99% sequence identity) to SEQ ID NO: 137. In some embodiments, the nucleotide sequence encoding the AAV capsid polypeptide or the AAV capsid variant comprises the nucleotide sequence of SEQ ID NO: 137 or a nucleotide sequence substantially identical (e.g., having at least 90%, at least 92%, at least 95%, at least 974, at least 98%, or at least 99% sequence identity) thereto. In some embodiments, the AAV capsid polypeptide or the AAV capsid variant, comprises substitution at position K449, e.g., a K449R substitution, numbered according to SEQ ID NO: 138.

In some embodiments, the AAV capsid polypeptide or the AAV capsid variant, comprises a peptide comprising the amino acid sequence of TLAVPFK (SEQ ID NO: 4680). In some embodiments, the peptide is present immediately subsequent to position 588, numbered according to SEQ ID NO: 138. In some embodiments, the capsid polypeptide comprises the amino acid substitutions of A587D and Q588G, numbered according to SEQ ID NO: 138.

In some embodiments, the AAV capsid polypeptide or the AAV capsid variant comprises the amino acid substitution of K449R, numbered according to SEQ ID NO: 138; and a peptide comprising the amino acid sequence of TLAVPFK (SEQ ID NO: 4680), wherein the peptide is present immediately subsequent to position 588, numbered according to SEQ ID NO: 138.

In some embodiments, the AAV capsid polypeptide or the AAV capsid variant comprises the amino acid substitution of K449R, numbered according to SEQ ID NO: 138; a peptide comprising the amino acid sequence of TLAVPFK (SEQ ID NO: 4680), wherein the peptide is present immediately subsequent to position 588, numbered according to SEQ ID NO: 138; and the amino acid substitutions of A587D and Q588G, numbered according to SEQ ID NO: 138.

In some embodiments, the AAV capsid polypeptide or the AAV capsid variant comprises a peptide comprising the amino acid sequence of TLAVPFK (SEQ ID NO: 4680), wherein the insert is present immediately subsequent to position 588, numbered according to SEQ ID NO: 138; and the amino acid substitutions of A587D and Q588G, numbered according to SEQ ID NO: 138.

In some embodiments, the AAV capsid polypeptide or the AAV capsid variant comprises the amino acid sequence of SEQ ID NO: 11 or an amino acid sequence substantially identical (e.g., having at least 70%, at least 75%, at least 80%, at least 85%, at least 90%, at least 92%, at least 95%, at least 97%, at least 98%, or at least 99% sequence identity) thereto. In some embodiments, the AAV capsid polypeptide or the AAV capsid variant comprises an amino acid sequence comprising at least one, two, or three modifications, e.g., substitutions (e.g., conservative substitutions), but no more than 30, no more than 20, or no more than 10 modifications, relative to the amino acid sequence of SEQ ID NO: 11, optionally wherein position 449 is not R.

In some embodiments, the AAV capsid polypeptide or AAV capsid variant comprises the amino acid sequence of SEQ ID NO: 1 or an amino acid sequence substantially identical (e.g., having at least 70%, at least 75%, at least 80%, at least 85%, at least 901%, at least 92%, at least 95%, at least 97%, at least 98% or at least 99% sequence identity) thereto. In some embodiments the AAV capsid polypeptide or the AAV capsid variant, comprises an amino acid sequence comprising at least one, two, or three modifications, but no more than 30, no more than 20, or no more than 10 modifications, relative to the amino acid sequence of SEQ ID NO: 1.

TABLE 6
AAV Sequences

| | SEQ | |
|---|---|---|
| | ID | |
| Serotype | NO | Sequence |
| VOY101 | 1 | MAADGYLPDWLEDNLSEGIREWWALKPGAPQPKANQQHQDNARGLVLPGYKYLGPGNGLD |
| | | KGEPVNAADAAALEHDKAYDQQLKAGDNPYLKYNHADAEFQERLKEDTSFGGNLGRAVFQ |
| | | AKKRLLEPLGLVEEAAKTAPGKKRPVEQSPQEPDSSAGIGKSGAQPAKKRINFGQTGDTE |
| | | SVPDPQPIGEPPAAPSGVGSLTMASGGGAPVADNNEGADGVGSSSGNWHCDSQWLGDRVI |
| | | TTSTRTWALPTYNNHLYKQISNSTSGGSSNDNAYEGYSTPWGYEDENRFHCHESPRDWQR |
| | | LINNNWGFRPKRLNFKLFNIQVKEVTDNNGVKTIANNLTSTVQVFTDSDYQLPYVLGSAH |
| | | EGCLPPFPADVEMIPQYGYLTLNDGSQAVGRSSFYCLEYFPSQMLRTGNNFQFSYEFENV |
| | | PFHSSYAHSQSLDRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHKEGEDRFFPLSGS |
| | | LIFGKQGTGRDNVDADKVMITNEEEIKTINPVATESYGQVATNHQSDGTLAVPFKAQAQT |
| | | GWVQNQGILPGMVWQDRDVYLQGPIWAKIPHTDGNFHPSPLMGGFGMKHPPPQILIKNTP |
| | | VPADPPTAENKDKLNSFITQYSTGQVSVEIEWELQKENSKRWNPEIQYTSNYYKSNNVEF |
| | | AVNTEGVYSEPRPIGTRYLTRNL |
| AAV9/hu.1 | 11 | MAADGYLPDWLEDNLSEGIREWWALKPGAPQPKANQQHQDNARGLVLPGYKYLGPGNGLD |
| 4 K449R | | KGEPVNAADAAALEHDKAYDQQLKAGDNPYLKYNHADAEFQERLKEDTSFGGNLGRAVFQ |
| | | AKKRLLEPLGIVEEAAKTAPGKKRPVEQSPQEPDSSAGIGKSGAQPAKKRLNFGQTGDTE |
| | | SVPDPQPIGEPPAAPSGVGSLTMASGGGAPVADNNEGADGVGSSSGNWHCDSQWLGDRVI |
| | | TTSTRTWALPTYNNHLYKQISNSTSGGSSNDNAYFGYSTPWGYFDFNRFHCHFSPRDWQR |
| | | LINNNWGFRPKRLNFKLENIQVKEVTDNNGVKTIANNLESTVQVFTDSDYQLPYVLGSAH |
| | | EGCLPPFPADVEMIPQYGYLTLNDGSQAVGRSSFYCLEYFPSQMLRTGNNFQFSYEFENV |
| | | PFHSSYAKSQSLDRLMNPLIDQYLYYLSRTINGSGQNQQTLKESVAGPSNMAVQGRNYIP |
| | | GPSYRQQRVSTTVTQNNNSEFAWPGASSWALNGRNSLMNPGPAMASHKEGEDRFFPLSGS |
| | | LIFGKQGTGRDNVDADKVMITNEEEIKTINPVATESYGQVATNHQSAQAQAQTGWVQNQG |
| | | ILPGMVWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAVNTEGV |
| | | YSEPRPIGTRYLTRND |
| AAV9/hu.1 | 138 | MAADGYLPDWLEDNLSEGIREWWALKPGAPQPKANQQHQDNARGLVLPGYKYLGPGNGLD |
| 4 WT | | KGEPVNAADAAALEHDKAYDQQLKAGDNPYLKYNHADAEFQERLKEDTSFGGNLGRAVEQ |
| (amino | | AKKRLLEPIGLVEEAAKTAPGKKRPVEQSPQEPDSSAGIGKSGAQPAKKRLNFGQTGDTE |
| acid) | | SVPDPQPIGEPPAAPSGVGSLTMASGGGAPVADNNEGADGVGSSSGNWHCDSQWLGDRVI |
| | | TTSTRTWALPTYNNHLYKQISNSTSGGSSNDNAYFGYSTPWGYEDENRFHCHESPRDWQR |
| | | LINNNWGFRPKRLNFKLFNIQVKEVTDNNGVKTIANNLTSTVQVFTDSDYQLPYVLGSAH |
| | | EGCLPPFPADVEMIPQYGYLTLNDGSQAVGRSSFYCLEYFPSQMLRIGNNFQFSYEFENV |
| | | PFHSSYAHSQSLDRLMNPLIDQYLYYLSKTINGSGQNQQTLKFSVAGPSNMAVQGRNYIP |
| | | GPSYRQQRVSTTVTQNNNSEFAWPGASSWALNGRNSLMNPGPAMASHKEGEDRFFPLSGS |
| | | LIFGKQGTGRDNVDADKVMITNEEEIKTINPVATESYGQVATNHQSAQAQAQTGWVQNQG |
| | | ILPGMVWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAVNTEGV |
| | | YSEPRPIGTRYLTRNL |
| AAV9/hu.1 | 137 | ATGGCTGCCGATGGTTATCTTCCAGATTGGCTCGAGGACAACCTTAGTGAAGGAATTCGC |
| 4 WT | | GAGTGGTGGGCTTTGAAACCTGGAGCCCCTCAACCCAAGGCAAATCAACAACATCAAGAC |
| (DNA) | | AACGCTCGAGGTCTTGTGCTTCCGGGTTACAAATACCTTGGACCCGGCAACGGACTCGAC |
| | | AAGGGGGAGCCGGTCAACGCAGCAGACGCGGCGGCCCTCGAGCACGACAAGGCCTACGAC |
| | | CAGCAGCTCAAGGCCGGAGACAACCCGTACCTCAAGTACAACCACGCCGACGCCGAGTTC |
| | | CAGGAGCGGCTCAAAGAAGATACGTCTTTTGGGGGCAACCTCGGGCGAGCAGTCTTCCAG |
| | | GCCAAAAAGAGGCTTCTTGAACCTCTTGGTCTGGTTGAGGAAGCGGCTAAGACGGCTCCT |
| | | GGAAAGAAGAGGCCTGTAGAGCAGTCTCCTCAGGAACCGGACTCCTCCGCGGGTATTGGC |
| | | AAATCGGGTGCACAGCCCGCTAAAAAGAGACTCAATTTCGGTCAGACTGGCGACACAGAG |
| | | TCAGTCCCAGACCCTCAACCAATCGGAGAACCTCCCGCAGCCCCCTCAGGTGTGGGATCT |
| | | CTTACAATGGCTTCAGGTGGTGGCGCACCAGTGGCAGACAATAACGAAGGTGCCGATGGA |
| | | GTGGGTAGTTCCTCGGGAAATTGGCATTGCGATTCCCAATGGCTGGGGGACAGAGTCATC |
| | | ACCACCAGCACCCGAACCTGGGCCCTGCCCACCTACAACAATCACCTCTACAAGCAAATC |
| | | TCCAACAGCACATCTGGAGGATCTTCAAATGACAACGCCTACTTCGGCTACAGCACCCCC |
| | | TGGGGGTATTTTGACTTCAACAGATTCCACTGCCACTTCTCACCACGTGACTGGCAGCGA |
| | | CTCATCAACAACAACTGGGGATTCCGGCCTAAGCGACTCAACTTCAAGCTCTTCAACATT |
| | | CAGGTCAAAGAGGTTACGGACAACAATGGAGTCAAGACCATCGCCAATAACCTTACCAGC |
| | | ACGGTCCAGGTCTTCACGGACTCAGACTATCAGCTCCCGTACGTGCTCGGGTCGGCTCAC |
| | | GAGGGCTGCCTCCCGCCGTTCCCAGCGGACGTTTTCATGATTCCTCAGTACGGGTATCTG |
| | | ACGCTTAATGATGGAAGCCAGGCCGTGGGTCGTTCGTCCTTTTACTGCCTGGAATATTTC |
| | | CCGTCGCAAATGCTAAGAACGGGTAACAACTTCCAGTTCAGCTACGAGTTTGAGAACGTA |
| | | CCTTTCCATAGCAGCTACGCTCACAGCCAAAGCCTGGACCGACTAATGAATCCACTCATC |
| | | GACCAATACTTGTACTATCTCTCAAAGACTATTAACGGTTCTGGACAGAATCAACAAACG |
| | | CTAAAATTCAGTGTGGCCGGACCCAGCAACATGGCTGTCCAGGGAAGAAACTACATACCT |
| | | GGACCCAGCTACCGACAACAACGTGTCTCAACCACTGTGACTCAAAACAACAACAGCGAA |
| | | TTTGCTTGGCCTGGAGCTTCTTCTTGGGCTCTCAATGGACGTAATAGCTTGATGAATCCT |
| | | GGACCTGCTATGGCCAGCCACAAAGAAGGAGAGGACCGTTTCTTTCCTTTGTCTGGATCT |
| | | TTAATTTTTGGCAAACAAGGAACTGGAAGAGACAACGTGGATGCGGACAAAGTCATGATA |
| | | ACCAACGAAGAAGAAATTAAAACTACTAACCCGGTAGCAACGGAGTCCTATGGACAAGTG |
| | | GCCACAAACCACCAGAGTGCCCAAGCACAGGCGCAGACCGGCTGGGTTCAAAACCAAGGA |
| | | ATACTTCCGGGTATGGTTTGGCAGGACAGAGATGTGTACCTGCAAGGACCCATTTGGGCC |
| | | AAAATTCCTCACACGGACGGCAACTTTCACCCTTCTCCGCTGATGGGAGGGTTTGGAATG |
| | | AAGCACCCGCCTCCTCAGATCCTCATCAAAAACACACCTGTACCTGCGGATCCTCCAACG |
| | | GCCTTCAACAAGGACAAGCTGAACTCTTTCATCACCCAGTATTCTACTGGCCAAGTCAGC |
| | | GTGGAGATCGAGTGGGAGCTGCAGAAGGAAAACAGCAAGCGCTGGAACCCGGAGATCCAG |
| | | TACACTTCCAACTATTACAAGTCTAATAATGTTGAATTTGCTGTTAATACTGAAGGTGTA |
| | | TATAGTGAACCCCGCCCCATTGGCACCAGATACCTGACTCGTAATCTGTAA |

### AAV Viral Genome

In some embodiments, the AAV particle of the present disclosure serves as an expression vector comprising a viral genome that encodes a STXBP1 protein (e.g., a wildtype STXBP1 protein. e.g., a wildtype human STXBP1 protein).

In some embodiments, an AAV particle, e.g., an AAV particle for the vectorized delivery of STXBP1 described herein, comprises a viral genome, e.g., an AAV viral genome (i.e., an AAV genome, a vector genome, or AA V vector genome). In some embodiments, the viral genome, e.g., the AAV viral genome, further comprises an inverted terminal repeat (ITR) region, an enhancer, a promoter, an intron region, an exon region, a nucleic acid encoding a transgene encoding a STXBP1 protein and a poly A region, or a combination thereof.

In some embodiments, the viral genome, e.g., the AAV viral genome, further comprises an inverted terminal repeat (ITR) region, an enhancer, a promoter, an intron region, an exon region, a nucleic acid encoding a transgene encoding a STXBP1 protein, and a poly A region, or a combination thereof. In some embodiments, the viral genome optionally comprises a nucleotide sequence encoding at least one miR binding site.

### Viral Genome Component: Inverted Terminal Repeats (ITRs)

In some embodiments, the viral genome may comprise at least one inverted terminal repeat (ITR) region. The AAV particles of the present disclosure comprise a viral genome with at least one ITR region and a region comprising a STXBP1-encoding sequence. In some embodiments, the viral genome has two ITRs. These two ITRs flank the region comprising the STXBP1-encoding sequence at the 5′ and 3′ ends. In some embodiments, the ITR functions as an origin of replication comprising a recognition site for replication. In some embodiments, the ITR comprises a sequence region which can be complementary and symmetrically arranged. In some embodiments, the ITR incorporated into a viral genome described herein may be comprised of a naturally occurring polynucleotide sequence or a recombinantly derived polynucleotide sequence.

In some embodiments, the ITR is of the same serotype as the capsid, selected from any of the serotypes known in the art, or a derivative thereof. In some embodiments, the ITR is of a different serotype than the capsid. In some embodiments, the AAV particle has more than one ITR. In some embodiments, the AAV particle comprises a viral genome comprising two ITRs. In some embodiments, the ITRs are of the same serotype as one another. In some embodiments, the ITRs are of different serotypes. Non-limiting examples include zero, one, or both of the ITRs having the same serotype as the capsid.

### Viral Genome Component: Promoters and Enhancers

In some embodiments, the viral genome comprises at least one element to enhance the transgene target specificity and expression. See, e.g., Powell et al. Viral Expression Cassette Elements to Enhance Transgene Target Specificity and Expression in Gene Therapy, 2015; the contents of which are herein incorporated by reference in their entirety. Non-limiting examples of elements to enhance the transgene target specificity and expression include promoters, endogenous miRNAs, post-transcriptional regulatory elements (PREs), polyadenylation (PolyA) sequences, upstream enhancers (USEs). CMV enhancers, and introns.

In some embodiments, expression of the polypeptides in a target cell may be driven by a specific promoter, including but not limited to, a promoter that is species specific, inducible, tissue-specific, or cell cycle-specific (Parr et at., Nat. Med. 3:1145-9 (1997); the contents of which are herein incorporated by reference in their entirety).

In some embodiments, the viral genome provides expression of a STXBP1 protein (e.g., a human STXBP1 protein encoded by a transgene for expression in a target tissue. In some embodiments, the promoter is deemed to be efficient when it drives expression of the STXBP1 encoded in the viral genome of the AAV particle.

In some embodiments, the promoter is a promoter deemed to be efficient when it drives expression in the cell or tissue being targeted (e.g., the CNS).

Promoters may be naturally occurring or non-naturally occurring. Non-limiting examples of promoters include viral promoters, plant promoters and mammalian promoters. In some embodiments, the promoters may be human promoters. In some embodiments, the promoters may be truncated.

In some embodiments, the viral genome comprises a promoter that results in expression in one or more cells and/or tissues. In some embodiments, the promoter is a ubiquitous promoter. In some embodiments, a promoter that drives or promotes expression in most mammalian tissues comprises a human elongation factor 1α-subunit (EF1α) promoter, a cytomegalovirus (CMV) immediate-early enhancer and/or promoter, a chicken β-actin (CBA) promoter, a CAG promoter, a β glucuronidase (GUSB) promoter, or a ubiquitin C (UBC) promoter.

In some embodiments, the viral genome comprises a nervous system-specific promoter. i.e., a promoter that results in expression of STXBP1 in a neuron, an astrocyte, and/or an oligodendrocyte. Non-limiting examples of tissue-specific expression elements for neurons include synapsin (Syn) or synapsin 1 (Syn1), e.g., human synapsin or synapsin 1.

In some embodiments, the promoter may be less than 1 kb.

In some embodiments, the promoter may be a combination of two or more components of the same or different starting or parental promoters.

In some embodiments, the viral genome comprises an enhancer.

In some embodiments, the viral genome comprises an engineered promoter.

### Viral Genome Component: Intron and Exon

In some embodiments, the AAV viral genome (i.e., AAV genome) comprises at least one intron or a fragment or derivative thereof. In some embodiments, the AAV viral genome comprises at least one exon or a fragment or derivative thereof.

In some embodiments, the intron may be 100-400 nucleotides in length.

In some embodiments, the STXBP1-encoding sequence may be located downstream of an intron such as a beta globin intron or others known in the art. In some embodiments, the STXBP1-encoding sequence may be located within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides downstream from a promoter comprising an intron (e.g., 3′ relative to the promoter comprising an intron) and/or upstream of the polyadenylation sequence (e.g., 5′ relative to the polyadenylation sequence). In some embodiments, the STXBP1-encoding sequence may be located within 1-5, 1-10, 1-15, 1-20, 1-25, 1-30, 5-10, 5-15, 5-20, 5-25, 5-30, 10-15, 10-20, 10-25, 10-30, 15-20, 15-25, 15-30, 20-25, 20-30, or 25-30 nucleotides downstream from the intron (e.g., 3′ relative to the intron) and/or upstream of the polyadenylation sequence (e.g., 5′ relative to the polyadenylation sequence). In some embodiments, the STXBP1-encoding sequence may be located within the first 1%. 2%, 3%, 4%. 5%, 6%. 7%, 8%, 9%, 10%, 15%, 2A %, 25%, or more than 25% of the nucleotides downstream from the intron (e.g., 3′ relative to the intron) and/or upstream of the polyadenylation sequence (e.g., 5′ relative to the polyadenylation sequence). In some embodiments, the STXBP1-encoding sequence may be located within the first 1-5%. 1-10%, 1-15%. 1-20%, 1-25%, 5-10%, 5-15%, 5-20%. 5-25%, 10-15%, 10-20%, 10-25%, 15-20%, 15-25%, or 20-25% of the sequence downstream from the intron (e.g., 3′ relative to the intron) and/or upstream of the polyadenylation sequence (e.g., 5′ relative to the polyadenylation sequence).

In certain embodiments, the intron sequence is not an enhancer sequence. In some embodiments, the intron sequence is not a sub-component of a promoter sequence. In some embodiments, the intron sequence is a sub-component of a promoter sequence.

### Viral Genome Component: Untranslated Regions (UTRs)

In some embodiments, a wildtype untranslated region (UTR) of a gene is transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs may be engineered into UTRs to enhance the stability and protein production.

In some embodiments, the viral genome encoding a transgene described herein (e.g., a transgene encoding a STXBP1 protein) comprises a Kozak sequence. While not wishing to be bound by theory, wild-type 5′ untranslated regions (UTRs) include features that play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In some embodiments, the 5′UTR in the viral genome includes a Kozak sequence.

In some embodiments, the 5′UTR in the viral genome does not include a Kozak sequence.

While not wishing to be bound by theory, wild-type 3′ UTRs are known to have stretches of adenosines and uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in their entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUU UA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In some embodiments, the 3′ UTR of the viral genome may include an oligo(dT) sequence for templated addition of a poly-A tail.

Any UTR from any gene known in the art may be incorporated into the viral genome of the AAV particle. These UTRs, or portions thereof, may be placed in the same orientation as in the gene from which they were selected or they may be altered in orientation or location. In some embodiments, the UTR used in the viral genome of the AAV particle may be inverted, shortened, lengthened, or made with one or more other 5′ UTRs or 3′ UTRs known in the art. As used herein, the term “altered.” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In some embodiments, the viral genome of the AAV particle comprises at least one artificial UTR, which is not a variant of a wild type UTR.

In some embodiments, the viral genome of the AAV particle comprises UTRs which have been selected from a family of transcripts whose proteins share a common function, stricture, feature, or property.

### Viral Genome Component: Polyadenylation Region

In some embodiments, the viral genome of the AAV particles of the present disclosure comprises at least one polyadenylation (poly A) region. The viral genome of the AAV particle may comprise a polyadenylation region that is positioned 3′ relative to the STXBP1-encoding sequence.

### Viral Genome Component: Filler (Stuffer) Sequence

As used herein, the terms “stuffer sequence” and “filler sequence” are used interchangeably. In some embodiments, the AAV particle viral genome comprises at least one filler sequence.

In some embodiments, the viral genome comprises one or more filler sequences. The filler sequence may be a wild-type sequence or an engineered sequence. A filler sequence may be a variant of a wild-type sequence.

In some embodiments, the viral genome comprises one or more filler sequences in order to have the length of the viral genome be the optimal size for packaging. In some embodiments, the viral genome comprises at least one filler sequence in order to have the length of the viral genome be about 2.3 kb. In some embodiments, the viral genome comprises at least one filler sequence in order to have the length of the viral genome be about 4.6 kb.

Viral Genome Component: STXBP1-encoding sequence

In some embodiments, the disclosure provides an AAV particle comprising a viral genome encoding a STXBP1 protein, e.g., a STXBP1 protein encoded by the nucleotide sequence of SEQ ID NO: 6414. In some embodiments, the viral genome comprises a promoter operably linked to a nucleotide sequence encoding a STXBP1 protein, e.g., a nucleotide sequence of SEQ ID NO: 6414.

In some embodiments, the disclosure herein provides constructs that allow for improved expression of STXBP1 protein delivered by gene therapy vectors.

In some embodiments, the disclosure provides constructs that allow for improved biodistribution of STXBP1 protein delivered by gene therapy vectors.

In some embodiments, the disclosure provides constructs that allow for improved sub-cellular distribution or trafficking of STXBP1 protein delivered by gene therapy vectors.

In some embodiments, the disclosure provides constructs that allow for improved trafficking of STXBP1 protein to lysosomal membranes delivered by gene therapy vectors.

In some embodiments, the present disclosure relates to a composition containing or comprising a nucleic acid sequence encoding a STXBP1 protein or a functional fragment or variant thereof and methods of administering the composition in vitro or in vivo in a subject, e.g., a human subject and/or an animal model of disease, e.g., a disease related to expression of STXBP1 (an STXBP1-related disorder).

In some embodiments, the nucleotide sequence comprises one or more, e.g., all of, a 5′ ITR sequence, an enhancer sequence, a promoter sequence, an intron sequence, a signal sequence, a STXBP-encoding sequence, a polyA sequence, and a 3′ ITR sequence. In some embodiments, the STXBP1 protein encoded by the nucleotide sequence encodes an amino acid sequence that is 100% identical to a wildtype STXBP1 protein.

In some embodiments, the AAV genome comprises a payload construct that comprises a combination of coding and non-coding nucleic acid sequences.

In some embodiments, the viral genome encodes more than one payload. As a non-limiting example, a viral genome encoding more than one payload may be replicated and packaged into a viral particle. A target cell transduced with a viral particle comprising more than one payload may express each of the payloads in a single cell.

In some embodiments, the STXBP1-encoding sequence comprises a gene therapy product including, but not limited to, a polypeptide, RNA molecule, or other gene product that, when expressed in a target cell, provides a desired therapeutic effect. In some embodiments, a gene therapy product may comprise a substitute for a non-functional gene or a gene that is absent, expressed in insufficient amounts, or mutated. In some embodiments, a gene therapy product may comprise a substitute for a non-functional protein or polypeptide or a protein or polypeptide that is absent, expressed in insufficient amounts, misfolded, degraded too rapidly, or mutated. For example, a gene therapy product may comprise a polynucleotide encoding a STXBP1 protein to treat STXBP1 deficiency or STXBP1-related disorders. In some embodiments, the gene therapy product comprises a polynucleotide sequence encoding a STXBP1 protein.

In some embodiments, the payload construct encodes a messenger RNA (mRNA), As used herein, the term “messenger RNA” (mRNA) refers to any polynucleotide that encodes a poly peptide of interest and that is capable of being translated to produce the encoded polypeptide of interest in vitro, in vivo, in situ, or ex vivo. Certain embodiments provide the mRNA as encoding STXBP1 or a variant thereof.

In some embodiments, a viral genome comprising an STXBP1-encoding sequence may further comprise or encode a selectable marker. In some embodiments, a selectable marker may comprise a gene sequence or a protein or polypeptide encoded by a gene sequence expressed in a host cell that allows for the identification, selection, and/or purification of the host cell from a population of cells that may or may not express the selectable marker. In some embodiments, the selectable marker provides resistance to survive a selection process that would otherwise kill the host cell, such as treatment with an antibiotic. In some embodiments, an antibiotic selectable marker may comprise one or more antibiotic resistance factors, including but not limited to neomycin resistance (e.g., neo), hygromycin resistance, kananycin resistance, and/or puromycin resistance.

In some embodiments the viral genome comprises a selectable marker including, but not limited to, β-lactamase, luciferase, β-galactosidase, or any other reporter gene as that term is understood in the art, including cell-surface markers, such as CD4 or the truncated nerve growth factor (NGFR) (for GFP, see WO %/23810; Heim el al., Current Biology 2:178-182 (996); Heim el al., Proc. Natl. Acad. Sci. US4 (1995); or Heim et al., Science 373:663-664 (1995); for β-lactamase, see WO 96/30540); the contents of each of which are herein incorporated by reference in their entirety.

In some embodiments, the viral genome encodes a selectable marker comprising a fluorescent protein. A fluorescent protein as herein described may comprise any fluorescent marker including but not limited to green, yellow, and/or red fluorescent protein (GFP, YFP, and/or RFP). In some embodiments the viral genome encodes a selectable marker comprising a human influenza hemagglutinin (HA) tag.

In certain embodiments, a nucleic acid for expression of a STXBP1 protein in a target cell as described herein will be incorporated into the viral genome and located between two ITR sequences.

### Viral Genome Component: Signal Sequence

In some embodiments, the viral genome encoding a STXBP1 protein further comprises a nucleotide sequence encoding a signal sequence.

In some embodiments, the nucleotide sequence encoding the signal sequence is located 5′ relative to the nucleotide sequence encoding the STXBP1 protein. In some embodiments, the encoded STXBP1 protein comprises a signal sequence at the N-terminus, wherein the signal sequence is optionally cleaved during cellular processing and/or localization of the STXBP1 protein.

### Exemplary STXBP1 Payload

In some embodiments, a viral genome, e.g., an AAV viral genome. AAV genome, or vector genome, described herein, comprises a promoter operably linked to a transgene encoding a STXBP1 protein. As used herein, a nucleotide sequence encoding a STXBP1 protein may also be referred to as a STXBP1-encoding sequence or a STXBP1 protein-encoding sequence.

In some embodiments, the encoded STXBP1 protein is a wildtype STXBP1 protein.

Tables 7 and 8 provide exemplary polynucleotide sequences encoding a STXBP1 protein and polypeptide sequences of exemplary STXBP1 proteins that may be used in the viral genomes disclosed herein and which may constitute a STXBP1 protein payload. In some embodiments, the STXBP1 protein suitable for delivery in an AAV disclosed herein is encoded by the nucleotide sequence of SEQ ID NO: 6437. In some embodiments, the STXBP1 protein suitable for delivery in an AAV disclosed herein is encoded by the nucleotide sequence of SEQ ID NO: 6416, 6418, 6420, 6422, 6424, 6426, 6428, 6430, 6432, 6434, or 6436.

TABLE 7
Exemplary STXBP1 Sequences

| SEQ ID | | | |
|---|---|---|---|
| NO: | Type | Species | Description |
| 6413 | Protein | Homo sapiens | STXBP1 protein NP_001027392.1 |
| | | | “syntaxin-binding protein 1 isoform b |
| | | | [Homo sapiens]”, National Center for |
| | | | Biotechnology Information, Mar. 18, 2023 |
| 6437 | DNA | Homo sapiens | STXBP1 mRNA transcript variant |
| | | | NM_001032221.6 |
| | | | “Homo sapiens syntaxin binding protein 1 |
| | | | (STXBP1), transcript variant 2, mRNA” |
| | | | National Center for Biotechnology |
| | | | Information, Dec. 2, 2019 |
| 6415 | Protein | Homo sapiens | STXBP1 protein NP_001361235.1 |
| | | | “syntaxin-binding protein 1 isoform c |
| | | | [Homo sapiens]”, National Center for |
| | | | Biotechnology Information, Mar. 18, 2023 |
| 6416 | DNA | Homo sapiens | STXBP1 mRNA transcript variant |
| | | | NM_{——}001374306.2 |
| | | | “Homo sapiens syntaxin binding protein 1 |
| | | | (STXBP1), transcript variant 3, mRNA” |
| | | | National Center for Biotechnology |
| | | | Information, Dec. 2, 2019 |
| 6417 | Protein | Homo sapiens | STXBP1 protein NP_001361236.1 |
| | | | “syntaxin-binding protein 1 isoform d |
| | | | [Homo sapiens]”, National Center for |
| | | | Biotechnology Information, Mar. 18, 2023 |
| 6418 | DNA | Homo sapiens | STXBP1 mRNA transcript variant |
| | | | NM_001374307.2 |
| | | | “Homo sapiens syntaxin binding protein 1 |
| | | | (STXBP1), transcript variant 4, mRNA” |
| | | | National Center for Biotechnology |
| | | | Information, Dec. 2, 2019 |
| 6419 | Protein | Homo sapiens | STXBP1 protein NP_001361237.1 |
| | | | “syntaxin-binding protein 1 isoform d |
| | | | [Homo sapiens]”, National Center for |
| | | | Biotechnology Information, Mar. 18, 2023 |
| 6420 | DNA | Homo sapiens | STXBP1 mRNA transcript variant |
| | | | NM_001374308.2 |
| | | | “Homo sapiens syntaxin binding protein 1 |
| | | | (STXBP1), transcript variant 5, mRNA” |
| | | | National Center for Biotechnology |
| | | | Information, Dec. 2, 2019 |
| 6421 | Protein | Homo sapiens | STXBP1 protein NP_001361238.1 |
| | | | “syntaxin-binding protein 1 isoform e |
| | | | [Homo sapiens]”, National Center for |
| | | | Biotechnology Information, Mar. 18, 2023 |
| 6422 | DNA | Homo sapiens | STXBP1 mRNA transcript variant |
| | | | NM_001374309.2 |
| | | | “Homo sapiens syntaxin binding protein 1 |
| | | | (STXBP1), transcript variant 6, mRNA” |
| | | | National Center for Biotechnology |
| | | | Information, Dec. 3, 2019 |
| 6423 | Protein | Homo sapiens | STXBP1 protein NP_001361239.1 |
| | | | “syntaxin-binding protein 1 isoform e |
| | | | [Homo sapiens]”, National Center for |
| | | | Biotechnology Information, Mar. 18, 2023 |
| 6424 | DNA | Homo sapiens | STXBP1 mRNA transcript variant |
| | | | NM_001374310.2 |
| | | | “Homo sapiens syntaxin binding protein 1 |
| | | | (STXBP1), transcript variant 7, mRNA” |
| | | | National Center for Biotechnology |
| | | | Information, Dec. 2, 2019 |
| 6425 | Protein | Homo sapiens | STXBP1 protein NP_001361240.1 |
| | | | “syntaxin-binding protein 1 isoform e |
| | | | [Homo sapiens]”, National Center for |
| | | | Biotechnology Information, Mar. 18, 2023 |
| 6426 | DNA | Homo sapiens | STXBP1 mRNA transcript variant |
| | | | NM_001374311.2 |
| | | | “Homo sapiens syntaxin binding protein 1 |
| | | | (STXBP1), transcript variant 8, mRNA” |
| | | | National Center for Biotechnology |
| | | | Information, Dec. 2, 2019 |
| 6427 | Protein | Homo sapiens | STXBP1 protein NP_001361241.1 |
| | | | “syntaxin-binding protein 1 isoform e |
| | | | [Homo sapiens]”, National Center for |
| | | | Biotechnology Information, Mar. 18, 2023 |
| 6428 | DNA | Homo sapiens | STXBP1 mRNA transcript variant |
| | | | NM_001374312.2 |
| | | | “Homo sapiens syntaxin binding protein 1 |
| | | | (STXBP1), transcript variant 9, mRNA” |
| | | | National Center for Biotechnology |
| | | | Information, Dec. 2, 2019 |
| 6429 | Protein | Homo sapiens | STXBP1 protein NP_001361242.1 |
| | | | “syntaxin-binding protein 1 isoform f |
| | | | [Homo sapiens]”, National Center for |
| | | | Biotechnology Information, Mar. 18, 2023 |
| 6430 | DNA | Homo sapiens | STXBP1 mRNA transcript variant |
| | | | NM_{——}001374313.2 |
| | | | “Homo sapiens syntaxin binding protein 1 |
| | | | (STXBP1), transcript variant 10, mRNA” |
| | | | National Center for Biotechnology |
| | | | Information, Dec. 2, 2019 |
| 6431 | Protein | Homo sapiens | STXBP1 protein NP_001361243.1 |
| | | | “syntaxin-binding protein 1 isoform g |
| | | | [Homo sapiens]”, National Center for |
| | | | Biotechnology Information, Mar. 18, 2023 |
| 6432 | DNA | Homo sapiens | STXBP1 mRNA transcript variant |
| | | | NM_001374314.1 |
| | | | “Homo sapiens syntaxin binding protein 1 |
| | | | (STXBP1), transcript variant 11, mRNA” |
| | | | National Center for Biotechnology |
| | | | Information, Mar. 17, 2023 |
| 6433 | Protein | Homo sapiens | STXBP1 protein NP_001361244.1 |
| | | | “syntaxin-binding protein 1 isoform h |
| | | | [Homo sapiens]”, National Center for |
| | | | Biotechnology Information, Mar. 18, 2023 |
| 6434 | DNA | Homo sapiens | STXBP1 mRNA transcript variant |
| | | | NM_001374315.2 |
| | | | “Homo sapiens syntaxin binding protein 1 |
| | | | (STXBP1), transcript variant 12, mRNA” |
| | | | National Center for Biotechnology |
| | | | Information, Dec. 2, 2019 |
| 6435 | Protein | Homo sapiens | STXBP1 protein NP_003156.1 |
| | | | “syntaxin-binding protein 1 isoform a |
| | | | [Homo sapiens]”, National Center for |
| | | | Biotechnology Information, Mar. 18, 2023 |
| 6436 | DNA | Homo sapiens | STXBP1 mRNA transcript variant |
| | | | NM_003165.6 |
| | | | “Homo sapiens syntaxin binding protein 1 |
| | | | (STXBP1), transcript variant 1, mRNA” |
| | | | National Center for Biotechnology |
| | | | Information, Dec. 2, 2019 |

TABLE 8
Exemplary STXBP1 Sequences

| Description | Sequence | SEQ ID NO: |
|---|---|---|
| STXBP1-nt | atggcccccattggcctcaaagctgttgtcggagagaagattatg | 6414 |
| | catgatgtgataaagaaggtcaagaagaagggggaatggaaggtg | |
| | ctggtggtggatcagttaagcatgaggatgctgtcctcctgctgc | |
| | aagatgacagacatcatgaccgagggcataacgattgtggaagat | |
| | atcaataagcgcagagagccgctccccagcctggaggctgtgtat | |
| | ctcatcactccatccgagaagtccgtccactctctcatcagtgac | |
| | tttaaggacccgccgactgctaaataccgggctgcacacgtcttc | |
| | ttcactgactcttgtccagatgccctgtttaatgaactggtaaaa | |
| | tcccgagcagccaaagtcatcaaaactctgacggaaatcaatatt | |
| | qcatttctcccgtatgaatcccaggtctattccttggactctgct | |
| | gactctttccaaagcttctacagtccccacaaggctcagatgaag | |
| | aatcctatactggagcgcctggcagagcagatcgcgaccctttgt | |
| | gccaccctgaaggagtacccggctgtgcggtatcggggggaatac | |
| | aaggacaatgccctgctggctcagctaatccaggacaagctcgat | |
| | gcctataaagctgatgatccaacaatgggggagggcccagacaag | |
| | gcacgctcccagctcctgatcctggatcgaggctttgaccccagc | |
| | tcccctgtgctccatgaattgacttttcaggctatgagttatgat | |
| | ctgctgcctatcgaaaatgatgtatacaagtatgagaccagcggc | |
| | atcggggaggcacgggtgaaggaggtgctcctggacgaggacgac | |
| | gacctgtggatagcactgcgccacaagcacatcgcagaggtgtcc | |
| | caggaagtcacccggtctctgaaagatttttcttctagcaagaga | |
| | atgaatactggagagaagaccaccatgcgggacctgtcccagatg | |
| | ctgaagaagatgcctcagtaccagaaagagctcagcaagtactcc | |
| | acccacctgcaccttgctgaggactgtatgaagcattaccaaggc | |
| | accgtagacaaactctgccgagtggagcaggacctggccatgggc | |
| | acagatgctgagggagagaagatcaaggaccctatgcgagccatc | |
| | gtccccattctgctggatgccaatgtcagcacttatgacaaaatc | |
| | cgcatcatccttctctacatctttttgaagaatggcatcacggag | |
| | gaaaacctgaacaaactgatccagcacgcccagatacccccggag | |
| | gatagtgagatcatcaccaacatggctcacctcggcgtgcccatc | |
| | gtcaccgattccacgctgcgtcgccggagcaagccggagcggaag | |
| | gaacgcatcagcgagcagacctaccagctctcacggtggactccg | |
| | attatcaaggacatcatggaggacactattgaggacaaacttgac | |
| | accaaacactacccttatatctctacccgttcctctgcctccttc | |
| | agcaccaccgccgtcagcgcccgctatgggcactggcataagaac | |
| | aaggccccaggcgagtaccgcagtggcccccgcctcatcattttc | |
| | atccttgggggtgtgagcctgaatgagatgcgctgcgcctacgag | |
| | gtgacccaggccaacggaaagtgggaggtgctgataggatccaca | |
| | cacatcctcaccccacagaaactgctggacacactgaagaaactg | |
| | aataaaacagatgaagaaataagcagttaa | |
| STXBP1-aa | MAPIGLKAVVGEKIMHDVIKKVKKKGEWKVLVVDQLSMRMLSSCC | 6413 |
| | KMTDIMTEGITIVEDINKRREPLPSLEAVYLITPSEKSVHSLISD | |
| | FKDPPTAKYRAAHVFFTDSCPDALFNELVKSRAAKVIKTITEINI | |
| | AFLPYESQVYSLDSADSFQSFYSPHKAQMKNPILERLAEQIATLC | |
| | ATLKEYPAVRYRGEYKDNALLAQLIQDKLDAYKADDPTMGEGPDK | |
| | ARSQLLILDRGFDPSSPVLHELTFQAMSYDLLPIENDVYKYETSG | |
| | IGEARVKEVLLDEDDDLWIALRHKHIAEVSQEVTRSLKDFSSSKR | |
| | MNTGEKTTMRDLSQMLKKMPQYQKELSKYSTHLHLAEDCMKHYQG | |
| | TVDKLCRVEQDLAMGTDAEGEKIKDPMRAIVPILLDANVSTYDKI | |
| | RIILLYIFLKNGITEENINKLIQHAQIPPEDSEIITNMAHLGVPI | |
| | VIDSTLRRRSKPERKERISEQTYQLSRWTPIIKDIMEDTIEDKLD | |
| | TKHYPYISTRSSASFSTTAVSARYGHWHKNKAPGEYRSGPRLIIF | |
| | ILGGVSLNEMRCAYEVTQANGKWEVLIGSTHILTPQKLLDTLKKL | |
| | NKTDEEISS | |

The exemplary STXBP1 sequences from the National Center for Biotechnology Information as cited in Table 7 are hereby incorporated by reference in their entirety.

In some embodiments, the present disclosure provides a viral genome comprising a STXBP1-encoding sequence comprising SEQ ID NO: 6414 or a nucleotide sequence having at least 75%, at least 80%, at least 85%, at least 90%, at least 91%, at least 92%, at least 93%, at least 94%, at least 95%, at least 9W % a at least 97%, at least 98%, or at least 99% sequence identity to SEQ ID NO: 6414. In some embodiments, the STXBP1-encoding sequence is at least 95% identical to SEQ ID NO: 6414. In some embodiments, the STXBP1-encoding sequence is at least 99% identical to SEQ ID NO: 6414. In some embodiments, the STXBP1-encoding sequence comprises SEQ ID NO: 6414. In some embodiments, the STXBP1-encoding sequence consists of SEQ ID NO: 6414. In some embodiments, the STXBP1 protein comprises or consists of the amino acid sequence of SEQ ID NO: 6413.

In some embodiments, the AAV viral genome further comprises a nucleic acid. In some embodiments, the AAV particle comprises a viral genome encoding a capsid protein, e.g., a structural protein. In some embodiments, the capsid protein comprises a VP1 polypeptide, a VP2 polypeptide, and/or a VP3 polypeptide. In some embodiments, the VP1 polypeptide, the VP2 polypeptide, and/or the VP3 polypeptide are encoded by at least one Cap gene. In some embodiments, the AAV viral genome further comprises a nucleic acid encoding a Rep protein, e.g., a non-structural protein. In some embodiments, the Rep protein comprises a Rep78 protein, a Rep68 protein, a Rep52 protein, and/or a Rep40 protein. In some embodiments, the Rep78 protein, the Rep68 protein, the Rep52 protein, and/or the Rep40 protein are encoded by at least one Rep gene.

In some embodiments, the AAV particle comprises a viral genome that is packaged in an AAV capsid variant comprising an amino acid sequence selected from Table 3 or Table 4. In some embodiments, the AAV capsid variant comprises the amino acid sequence of HDSPHK (SEQ ID NO: 2), which is present in loop IV, e.g., between amino acids 449460 numbered according to SEQ ID NO: 982 (i.e., at a sequence position corresponding to that in SEQ ID NO: 982). In some embodiments, the AAV capsid variant comprises: (i) a VP1 protein comprising or consisting of the amino acid sequence of SEQ ID NO: 982 or an amino acid sequence that is at least 95% identical (e.g., at least 95%, at least 96%, at least 97%, at least 98%, or at least 99% identical) thereto; (ii) a VP2 protein comprising or consisting of the amino acid sequence according to positions 138-742 of SEQ ID NO: 982 or an amino acid sequence that is at least 95% identical (e.g., at least 95%, at least 96%, at least 97%, at least 98%, or at least 99% identical) thereto; and/or (iii) a VP3 protein comprising or consisting of the amino acid sequence according to positions 203-742 of SEQ ID NO: 982 or an amino acid sequence that is at least 95% identical (e.g., at least 95%, at least 96%, at least 97%, at least 98%, or at least 99% identical) thereto.

In some embodiments, the AAV particle comprises a viral genome that is packaged in an AAV capsid variant comprising an amino acid sequence selected from Table 3 or Table 4. In some embodiments, the AAV capsid variant comprises the amino acid sequence of SPHSKA (SEQ ID NO: 941), which is present in loop IV, e.g., between amino acids 449-460 numbered according to SEQ ID NO: 36 (i.e., at a sequence position corresponding to that in SEQ ID NO: 36). In some embodiments, the AAV capsid variant comprises the amino acid E at position 451, the amino acid R at position 452, and the amino acid V at position 453, numbered according to SEQ ID NO: 36. In some embodiments, the AAV capsid variant comprises: (i) the amino acid sequence of SPHSKA (SEQ ID NO: 941) present immediately subsequent to position 455 (e.g., at positions 456-461), numbered according to SEQ ID NO: 36; and (ii) the amino acid E at position 451, the amino acid R at position 452, and the amino acid V at position 453, numbered according to SEQ ID NO: 36. In some embodiments, the AAV capsid variant comprises the amino acid sequence of KTERVSGSPHSKAQNQQT (SEQ ID NO: 3589) in loop TV, e.g., between amino acids 449460 numbered according to SEQ ID NO: 36. In some embodiments, the AAV capsid variant comprises: (i) a VP1 protein comprising or consisting of the amino acid sequence of SEQ ID NO: 36 or an amino acid sequence that is at least 95% identical (e.g., at least 95%, at least 96%, at least 97%, at least 98%, or at least 99% identical) thereto: (ii) a VP2 protein comprising or consisting of the amino acid sequence according to positions 138-742 of SEQ ID NO: 36 or an amino acid sequence that is at least 95% identical (e.g., at least 95%, at least 96%, at least 97%, at least 984, or at least 99% identical) thereto; and/or (iii) a VP3 protein comprising or consisting of the amino acid sequence according to positions 203-742 of SEQ ID NO: 36 or an amino acid sequence that is at least 95% identical (e.g., at least 95%, at least 96%, at least 97%, at least 98%, or at least 99% identical) thereto.

In some embodiments, the AAV particle comprises a viral genome that is packaged in an AAV capsid variant comprising an amino acid sequence selected from Table 3 or Table 4. In some embodiments, the AAV capsid variant comprises the amino acid sequence of SPSHKA (SEQ ID NO: 941), which is present in loop TV, e.g., between amino acids 449460 numbered according to SEQ ID NO: 4 (i.e., at a sequence position corresponding to that in SEQ ID NO: 4). In some embodiments, the AAV capsid variant comprises the amino acid E at position 451, and the amino acid V at position 453, numbered according to SEQ ID NO: 4. In some embodiments, the AAV capsid variant comprises: (i) the amino acid sequence of SPHSKA (SEQ ID NO: 941) present immediately subsequent to position 455 (e.g., at positions 456-461), numbered according to SEQ ID NO: 4; and (ii) the amino acid E at position 451 and the amino acid V at position 453, numbered according to SEQ ID NO: 4. In some embodiments, the AAV capsid variant comprises the amino acid sequence of KTENVSGSPHSKAQNQQT (SEQ ID NO: 3272) in loop IV, e.g., between amino acids 449460 numbered according to SEQ ID NO: 4. In some embodiments, the AAV capsid variant comprises: (i) a VP1 protein comprising or consisting of the amino acid sequence of SEQ ID NO: 4 or an amino acid sequence that is at least 95% identical (e.g., at least 95%, at least 96%, at least 97%, at least 98%, or at least 99% identical) thereto; (ii) a VP2 protein comprising or consisting of the amino acid sequence according to positions 138-742 of SEQ ID NO: 4 or an amino acid sequence that is at least 95% identical (e.g., at least 95%, at least 96%, at least 97%, at least 98%, or at least 99% identical) thereto; and/or (iii) a VP3 protein comprising or consisting of the amino acid sequence according to positions 203-742 of SEQ ID NO: 4 or an amino acid sequence that is at least 95% identical (e.g., at least 950, at least 96%, at least 97%, at least 98%, or at least 99% identical) thereto.

In some embodiments, the AAV particle comprises a viral genome comprising the nucleotide sequence of SEQ ID NO: 6414, or a sequence that is at least 90% identical (e.g., at least 90%, at least 91%, at least 92%, at least 93%, at least 94%, at least 95%, at least 96%, at least 97%, at least 98%, or at least 99% identical) thereto.

In some embodiments, the AAV particle comprising the viral genome comprising the nucleotide sequence of SEQ ID NO: 6414 or a sequence that is at least 90% identical (e.g., at least 90%, at least 91%, at least 92%, at least 93%, at least 94%, at least 95%, at least 96%, at least 97%, at least 98%, or at least 99% identical) thereto, comprises an AAV capsid variant comprising an amino acid sequence selected from Table 3 or Table 4.

In some embodiments, the AAV particle comprising the viral genome comprising the nucleotide sequence of SEQ ID NO: 6414, or a sequence having at least 90% identity (e.g., at least 90%, at least 91%, at least 92%, at least 93%, at least 94%, at least 95%, at least 96%, at least 97%, at least 98%, or at least 99% identity) thereto, comprises an AAV capsid variant comprising: (i) a VP1 protein comprising or consisting of the amino acid sequence of SEQ ID NO: 982 or an amino acid sequence that is at least 95% identical (e.g., at least 95%, at least 96%, at least 97%, at least 98%, or at least 99% identical) thereto; (ii) a VP2 protein comprising or consisting of the amino acid sequence of positions 138-742 of SEQ ID NO: 982 or an amino acid sequence that is at least 95% identical (e.g., at least 95%, at least 96%, at least 97%, at least 98%, or at least 99% identical) thereto; and/or (iii) a VP3 protein comprising or consisting of the amino acid sequence of positions 203-742 of SEQ ID NO: 982 or an amino acid sequence that is at least 95% identical (e.g., at least 95%, at least 96%, at least 97%, at least 98%, or at least 99% identical) thereto.

In some embodiments, the AAV particle comprising the viral genome comprising the nucleotide sequence of SEQ ID NO: 6414, or a sequence having at least 90% identity (e.g., at least 90%, at least 91%, at least 92%, at least 93%, at least 94%, at least 95%, at least 96%, at least 97%, at least 98%, or at least 99% identity) thereto, comprises an AAV capsid variant comprising: (i) a VP1 protein comprising or consisting of the amino acid sequence of SEQ ID NO: 36 or an amino acid sequence that is at least 95% identical (e.g., at least 95%, at least 96%, at least 97%, at least 98%, or at least 99% identical) thereto; (ii) a VP2 protein comprising or consisting of the amino acid sequence of positions 138-742 of SEQ ID NO: 36 or an amino acid sequence that is at least 95% identical (e.g., at least 95%, at least %, at least 97%, at least 98%, or at least 99% identical) thereto; and/or (iii) a VP3 protein comprising or consisting of the amino acid sequence of positions 203-742 of SEQ ID NO: 36 or an amino acid sequence that is at least 95% identical (e.g., at least 95%, at least 96%, at least 97%, at least 98%, or at least 99% identical) thereto.

In some embodiments, the AAV particle comprising the viral genome comprising the nucleotide sequence of SEQ ID NO: 6414 or a sequence having at least 90% identity (e.g., at least 90%, at least 91%, at least 92%, at least 93%, at least 94%, at least 95%, at least 96%, at least 97%, at least 98%, or at least 99% identity) thereto, comprises an AAV capsid variant comprising: (i) a VP1 protein comprising or consisting of the amino acid sequence of SEQ ID NO: 4 or an amino acid sequence that is at least 95% identical (e.g., at least 95%, at least %, at least 97%, at least 98%, or at least 99% identical) thereto; (ii) a VP2 protein comprising or consisting of the amino acid sequence of positions 138-742 of SEQ ID NO: 4 or an amino acid sequence that is at least 95% identical (e.g., at least 95%, at least 96%, at least 97%, at least 98%, or at least 99% identical) thereto; and/or (iii) a VP3 protein comprising or consisting of the amino acid sequence of positions 203-742 of SEQ ID NO: 4 or an amino acid sequence that is at least 95% identical (e.g., at least 95%, at least 96%, at least 97%, at least 98%, or at least 99% identical) thereto.

In some embodiments, the AAV particle comprises an AAV capsid variant and a nucleic acid encoding a STXBP1 protein, wherein the AAV capsid variant comprises an amino acid sequence having the following formula: [N1]-[N2]-[N3], wherein: (i) [N1] comprises X1, X2, and X3, wherein at least one of X1, X2, or X3 is G; (ii) [N2] comprises the amino acid sequence of SPH; and; wherein [N1]-[N2]-[N3] is present in hypervariable loop IV; and wherein the AAV capsid variant comprises an amino acid sequence at least 95% identical to the amino acid sequence of positions 203-736 of SEQ ID NO: 138. In some embodiments, the STXBP1-encoding sequence comprises the nucleotide sequence of SEQ ID NO: 6414 or a sequence having at least 90% identity (e.g., at least 90%, at least 91%, at least 92%, at least 93%, at least 94%, at least 95%, at least 96%, at least 97%, at least 98%, or at least 99% identity) thereto.

The present disclosure provides, in some embodiments, vectors, cells, and/or AAV particles comprising the above-identified viral genomes and/or capsid variants.

### Self-Complementary and Single Strand Vectors

In some embodiments, the AAV viral genome used in the present disclosure is single-stranded (ssAAV).

In some embodiments, the AAV viral genome is capable of forming double-stranded DNA. In some embodiments, the AAV viral genome is self-complementary. See, e.g., U.S. Pat. No. 7,465,583, scAAV particles contain both DNA strands that anneal together to form double stranded DNA. By skipping second strand synthesis, scAAVs allow for rapid expression in the cell.

Methods for producing and/or modifying AAV viral genome and particles are disclosed in the art such as pseudotyped AAV particles (international Patent Publication Nos. WO200028004; WO200123001; WO2004112727; WO 2005005610 and WO 2005072364, the content of each of which are incorporated herein by reference in their entirety).

### II. AAV Production

Viral production disclosed herein describes processes and methods for producing AAV particles (with enhanced, improved and/or increased tropism for a target tissue), e.g., an AAV particle comprising an AAV capsid variant that may be used to contact a target cell to deliver STXBP1.

In some embodiments, disclosed herein is a method of making an AAV particle of the present disclosure, e.g., an AAV particle comprising an AAV capsid variant disclosed herein, wherein the method comprises: (i) providing a cell comprising a viral genome comprising a STXBP1-encoding sequence and a nucleic acid encoding an AAV capsid variant; and (ii) incubating the cell under conditions suitable to encapsulate the viral genome in the AAV capsid variant: thereby making the AAV particle. In some embodiments, the viral genome comprises the nucleotide sequence of SEQ ID NO: 6414 or a nucleotide sequence at least 90% identical (e.g., at least 90%, at least 91%, at least 92%, at least 93%, at least 94%, at least 95%, at least 96%, at least 97%, at least 98%, or at least 99% identical) thereto; and wherein the AAV capsid variant comprises the amino acid sequence of SEQ ID NO: 982, 36, or 4. In some embodiments, the method comprises, prior to step (i), introducing a nucleic acid comprising the viral genome into the cell. In some embodiments, the method comprises, prior to step (i), introducing the nucleic acid encoding the AAV capsid variant into the cell. In some embodiments, the cell comprises a mammalian cell (e.g., an HEK293 cell), an insect cell (e.g., an Sf9 cell), or a bacterial cell. In some embodiments, the AAV particle described herein is an isolated AAV particle. In some embodiments, the AAV particle described herein is a recombinant AAV particle.

Any method known in the art may be used for the preparation of AAV particles. In some embodiments, AAV particles are produced in mammalian cells (e.g., HEK293 cells). In some embodiments, AAV particles are produced in insect cells (e.g., Sf9 cells).

Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764. U.S. Pat. Nos. 6,194,191, 6,566,118, 8,137,948: or International Publication Nos. WO1996039530, WO1998010088, WO1999014354, WO1999015685, WO1999047691, WO2000055342, WO2000075353 and WO201023597; Methods In Molecular Biology, ed. Richard. Humana Press. NJ (1995); O'Reilly et al., Baculovirus Expression Vectors, A Laboratory Manual. Oxford Univ. Press (1994); Samulski et al., J. Vir.63:3822-8 (1989); Kajigaya et al., Proc. Natl. Acad. Sci. US4 88: 4646-50 (1991): Ruffing et al., J. Vir. 66:6922-30 (1992); Kimbauer et al., Virl., 219:37-44 (1996); Zhao et al., Vir.272:382-93 (2000); the contents of each of which are herein incorporated by reference in their entirety. In some embodiments, the AAV particles are made using the methods described in International Patent Publication WO2015191508, the contents of which are herein incorporated by reference in their entirety.

### III. Pharmaceutical Compositions

The present disclosure additionally provides a method for treating STXBP1-related disorders in a subject, e.g., in a human subject, comprising administering to the subject any of the AAV polynucleotides or AAV genomes described herein or administering to the subject a particle comprising said AAV polynucleotide or AAV genome, or administering to the subject any of the described compositions, including pharmaceutical compositions.

In some embodiments, a composition described herein comprises an AAV polynucleotide or AAV genome or AAV particle and at least one excipient.

Although pharmaceutical compositions provided herein, e.g., comprising AAV particles comprising a STXBP1-encoding sequence, are principally directed to pharmaceutical compositions that are suitable for administration to humans, it will be understood by the skilled artisan that such compositions may be suitable for administration to any other animal, e.g., non-human mammals. Modification of pharmaceutical compositions suitable for administration to humans in order to render the compositions suitable for administration to various non-human animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In some embodiments, compositions are administered to humans, e.g., human patients or human subjects.

In some embodiments, the AAV particle formulations described herein may contain a nucleic acid encoding at least one STXBP1-encoding sequence. In some embodiments, the formulations may contain a nucleic acid encoding 1, 2, 3, 4, or 5 STXBP1-encoding sequences. In some embodiments, the formulation may contain a STXBP1-encoding sequences, encoding STXBP1 proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic proteins, cytoskeletal proteins, intracellular membrane bound proteins, nuclear proteins, proteins associated with human disease, and/or proteins associated with non-human diseases. In some embodiments, the AAV genome comprises at least one sequence encoding human STXBP1. In some embodiments, the AAV genome comprises at least one sequence encoding wildtype human STXBP1.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

### IV. Formulations

Formulations of the AAV pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered.

For example, the composition may comprise about 0.1% to about 99% (w/w) of the active ingredient. By way of example, the composition may comprise about 0.1% to about 100%, e.g., about 0.5% to about 50%, about 1% to about 30%, about 5% to about 80%, or at least 80% (w/w) active ingredient.

The AAV particles of the disclosure can be formulated using one or more excipients to; (1) increase stability: (2) increase cell transfection or transduction: (3) permit the sustained or delayed release; (4) alter the biodistribution (e.g., target the viral particle to specific tissues or cell types); (5) increase the translation of encoded protein in vivo; (6) alter the release profile of encoded protein in vivo and/or (7) allow for regulatable expression of STXBP1.

Formulations of the present disclosure can include, without limitation, saline, lipidoids, liposomes, lipid nanoparticles, polymers, lipoplexes, core-shell nanoparticles, peptides, proteins, cells transfected with viral vectors (e.g., for transplantation into a subject), nanoparticle mimics and combinations thereof. Further, the viral vectors of the present disclosure may be formulated using self-assembled nucleic acid nanoparticles.

In some embodiments, the viral vectors encoding STXBP1 may be formulated to optimize baricity and/or osmolality. In sonic embodiments, the baricity and/or osmolality of the formulation may be optimized to ensure optimal drug distribution in the central nervous system or a region or component of the central nervous system.

### Excipients

The formulations of the disclosure can include one or more excipients, each in an amount that together increases the stability of the AAV particle, increases cell transfection or transduction by the viral particle, increases the expression of STXBP1, and/or alters the release profile of STXBP1. In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, which, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition. A, R, Gennaro. Lippincott, Williams & Wilkins, Baltimore. MD, 2006; the contents of which are herein incorporated by reference in their entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Inactive Ingredients

In some embodiments, AAV formulations may comprise at least one excipient which is an inactive ingredient. As used herein, the term “inactive ingredient” refers to one or more agents that do not contribute to the activity of the pharmaceutical composition included in formulations. In some embodiments, all, none, or some of the inactive ingredients which may be used in the formulations of the present disclosure may be approved by the US Food and Drug Administration (FDA).

Formulations of AAV particles may include cations or anions. In some embodiments, the formulations include metal cations such as, but not limited to, Zn²⁺, Ca²⁺, Cu²⁺, Mg⁺, or combinations thereof. In one embodiment, formulations may include polymers or polynucleotides complexed with a metal cation (see, e.g., U.S. Pat. Nos. 6,265,389 and 6,555,525, the contents of each of which are herein incorporated by reference in their entirety).

### V. Uses and Applications

The compositions of the disclosure may be administered to a subject, e.g., to deliver STXBP1, e.g., to a subject who has, has been diagnosed with having, or is at risk of having a STXBP1-related disorder. In some embodiments, the STXBP1-related disorder is a neurodegenerative or neuromuscular disorder. In some embodiments, the STXBP1-related disorder is STXBP1 encephalopathy, epileptic encephalopathy, Ohtahara syndrome, developmental encephalopathy. West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5)). The compositions may similarly be used in the manufacture of a medicament for administration to a subject having a STXBP1-related disorder (e.g., a STXBP1-related neurodegenerative or neuromuscular disorder). In some embodiments, the STXBP1-related disorder is STXBP1 encephalopathy.

In some embodiments, the disclosure provides a method of delivering a STXBP1 protein to a subject comprising administering to the subject an effective amount of a pharmaceutical composition or AAV particle disclosed herein that comprises a viral genome encoding a STXBP1 protein, thereby delivering the STXBP1 protein. In sonic embodiments, the subject has, has been diagnosed with having, or is at risk of having a STXBP1-related disorder. The STXBP1-related disorder may be a STXBP1-related disorder such as a STXBP1-related neurodegenerative or neuromuscular disorder (e.g., STXBP1 encephalopathy, epileptic encephalopathy. Ohtahara syndrome, developmental encephalopathy. West syndrome, early myoclonic epileptic encephalopathy. Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5)). In some embodiments, the STXBP1-related disorder is STXBP1 encephalopathy.

In some embodiments, the disclosure provides a method for treating a STXBP1-related disorder such as a STXBP1-related neurodegenerative or neuromuscular disorder. In some embodiments, the disclosure provides an AAV particle or pharmaceutical composition according to any one of the embodiments disclosed herein for treating a STXBP1-related disorder, such as STXBP1 encephalopathy, epileptic encephalopathy, Ohtahara syndrome, developmental encephalopathy. West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5). In some embodiments, the present disclosure provides the pharmaceutical composition or the AAV particle of any one the embodiments disclosed herein for use in a method of treating a disorder as disclosed herein, such as STXBP1 encephalopathy, epileptic encephalopathy, Ohtahara syndrome, developmental encephalopathy, West syndrome, early myoclonic epileptic encephalopathy, Lennox-Gaustaut syndrome, autism (e.g., autism with STXBP1 mutations and optionally further mutations). Dravet syndrome (not caused by mutations in SCN1A), or Rett syndrome phenotype (not caused by mutation of MECP2 or CDKL5). In some embodiments, the disorder is STXBP1 encephalopathy.

In certain embodiments, a pharmaceutical composition or AAV particle disclosed herein that comprises a viral genome encoding STXBP1 may be administered to a subject to treat a STXBP1-related disorder such as a STXBP1-related neurodegenerative or neuromuscular disorder, thereby treating the STXBP1-related disorder. In some embodiments, the subject has, has been diagnosed with having, or is at risk of having a STXBP1-related disorder such as a STXBP1-related neurodegenerative or neuromuscular disorder (e.g., STXBP1 encephalopathy, epileptic encephalopathy, Ohtahara syndrome, developmental encephalopathy. West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5)). In some embodiments, the treatment results in an increase in the subject's STXBP1 protein level as compared to baseline.

In some embodiments, the STXBP1-related disorder is STXBP1 encephalopathy, epileptic encephalopathy. Ohtahara syndrome, developmental encephalopathy, West syndrome, early myoclonic epileptic encephalopathy. Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5). In some embodiments, the STXBP1-related disorder is STXBP1 encephalopathy.

In some embodiments, the disclosure provides a method of treating STXBP1 encephalopathy in a subject. In certain embodiments, a pharmaceutical composition or AAV particle disclosed herein that comprises a viral genome encoding STXBP1 may be administered to a subject to treat STXBP1 encephalopathy. In some embodiments, the subject has, has been diagnosed with having, or is at risk of having STXBP1 encephalopathy.

A subject may have one or more mutations in the STXBP1 gene. In some embodiments, the subject has lower STXBP1 activity as compared to STXBP1 activity in an individual who does not have a STXBP1-related disorder.

In some embodiments, the treatment may result in prevention of progression of the STXBP1-related disorder. For example, the treatment may result in in amelioration of at least one symptom of the disorder, and/or a change in one or more biomarkers. In some embodiments, the one or more biomarkers comprises increased release of the neurotransmitter glutamate and/or GABA, reduction in accumulation of neurofilament light chain (e.g., in a biofluid such as cerebrospinal fluid), or reduction in abnormal electroencephalographic activity as evidence of improved STXBP1 activity.

In certain embodiments, the treatment improves at least one symptom of a STXBP1-related disorder, e.g., epilepsy, autistic features, ataxia, generalized tremors, reduced STXBP1 activity, accumulation of glucocerebroside and other glycolipids, e.g., within immune cells (e.g., macrophages), build-up of synuclein aggregates (e.g., Lewy bodies), developmental delay, progressive encephalopathy, progressive dementia, ataxia, myoclonus, oculomotor dysfunction, bulbar palsy, generalized weakness, trembling of a limb, depression, visual hallucinations, cognitive decline, dystonia, or a combination thereof.

In some embodiments, the methods disclosed herein further comprise evaluating, e.g., measuring, the level of STXBP1 expression, e.g., STXBP1 gene expression, STXBP1 mRNA expression, and/or STXBP1 protein expression, in the subject, e.g., in a cell, tissue, or fluid of the subject. STXBP1 protein expression may be measured by an ELISA, a Western blot, or an immunohistochemistry assay. In some embodiments, evaluating the level of STXBP1 expression is performed prior to and/or subsequent to administration of the AAV particle, optionally wherein the level of STXBP1 expression prior to treatment is compared to the level of STXBP1 expression subsequent to administration. In some embodiments, the level of STXBP1 expression may be evaluated in a cell or tissue of the central nervous system. In some embodiments, the level of STXBP1 expression may be evaluated in a cell of a peripheral tissue (e.g., liver, heart, muscle, or spleen). In some embodiments, the cell of a peripheral tissue is a muscle cell. In some embodiments, the subject's level of STXBP1 protein expression subsequent to administration is increased relative to the subject's level of STXBP1 protein expression prior to administration.

In some embodiments, the administration of the effective amount of a pharmaceutical composition or AAV particle disclosed herein that comprises a viral gnome encoding a STXBP1 protein may be a treatment that results in an increase in: (i) the level of STXBP1 activity in a cell, tissue, (e.g., a cell or tissue of the CNS, e.g., the cortex, striatum, thalamus, cerebellum, and/or brainstem), and/or fluid (e.g., CSF and/or serum), of the subject, relative to baseline and/or relative to the level of STXBP1 activity in a cell, tissue, or fluid of an individual with a STXBP1-related disorder who has not been administered the pharmaceutical composition or AAV particle; and/or (ii) the level and/or number of viral genomes (VG) per cell in a tissue of the CNS, e.g., the cortex, striatum, thalamus, cerebellum, and/or brainstem), of the subject relative to the number and/or level of VG per cell in a peripheral tissue of the subject; and/or (iii) the level of STXBP1 mRNA expression in a cell or tissue (e.g., a cell or tissue of the CNS, e.g., the cortex, striatum, thalamus, cerebellum, and/or brainstem), of the subject relative to baseline and/or relative to the level of STXBP1 mRNA expression in a cell or tissue of an individual with a STXBP1-related disorder who has not been administered the pharmaceutical composition or AAV particle.

In some embodiments, at least one additional agent suitable for treatment of a STXBP1-related disorder may be administered together with the effective amount of a pharmaceutical composition or AAV particle disclosed herein (comprising a viral genome encoding a STXBP1 protein). The at least one additional agent may comprise one or more anti-epileptic drugs (e.g., bromide, clobazam, felbamate, ganaxolone, lamotrigine, levetiracetam, phenobarbital, topiramate, valproate, or a combination thereof).

In some embodiments, the present disclosure encompasses the delivery of pharmaceutical, prophylactic, diagnostic, or imaging compositions in combination with agents that may improve their bioavailability, reduce and/or modify their metabolism, and/or modify their distribution within the body.

In certain embodiments, the pharmaceutical compositions described herein are used as research tools, particularly in in vitro investigations using human cell lines such as HEK293T and in vivo testing in nonhuman primates which will occur prior to human clinical trials.

The present disclosure provides a method for treating a disease, disorder and/or condition in a mammalian subject, including a human subject, comprising administering to the subject any of the viral particles, e.g., AAV, AAV particles, or AAV genome that produces STXBP1 protein described herein (i.e., viral genomes or “VG”), administering to the subject a composition or formulation comprising said AAV particle.

Delivery of a payload construct comprising a STXBP1-encoding sequence may alleviate or reduce symptoms that result from abnormal level and/or function of a gene product (e.g., an absence or defect in a protein) in a subject in need thereof or that otherwise confers a benefit to a CNS disorder in a subject in need thereof.

### VI. Delivery of AAV Particles

### Delivery to Cells

In some aspects, the present disclosure provides a method of delivering to a cell or tissue any of the above-described AAV particles, comprising contacting the cell or tissue with said AAV particle or contacting the cell or tissue with a formulation comprising said AAV particle, or contacting the cell or tissue with any of the described compositions, including pharmaceutical compositions. The method of delivering the AAV particle to a cell or tissue can be accomplished in vitro, ex vivo, or in vivo.

In some embodiments, the AAV particles are delivered to a cell, tissue, or region of the CNS. In some embodiments, the AAV particles are delivered to a cell or tissue of the CNS, e.g., the cortex, striatum, thalamus, cerebellum, brainstem, and/or spinal cord), and/or fluid (e.g., CSF and/or serum) of the subject. In some embodiments, the AAV particles are delivered to a cell or tissue of the thalamus, hippocampus, cerebellum, and/or striatum.

### Delivery to Subjects

In some aspects, the present disclosure additionally provides a method of delivering to a subject, including a mammalian subject, any of the above-described AAV particles comprising administering to the subject said AAV particle, or administering to the subject a formulation comprising said AAV particle, or administering to the subject am of the described compositions, including pharmaceutical compositions.

In some embodiments, the AAV particles may be delivered to bypass anatomical blockages (e.g., the blood brain barrier).

In some embodiments, the AAV particles may be formulated and delivered to a subject by a route which increases the speed of drug effect as compared to oral delivery.

In some embodiments, the AAV particles may be delivered using intrathecal infusion.

In some embodiments, a subject may be administered the AAV particles described herein using a bolus infusion.

In some embodiments, the AAV particles may be delivered in a continuous and/or bolus infusion. Each site of delivery may use a different dosing regimen or the same dosing regimen may be used for each site of delivery. As a non-limiting example, the sites of delivery may be in the cervical and the lumbar region. As another non-limiting example, the sites of delivery may be in the cervical region. As another non-limiting example, the sites of delivery may be in the lumbar region.

In some embodiments, the AAV particles may be delivered to a subject via a single route of administration.

In some embodiments, the AAV particles may be delivered to a subject via a multi-site route of administration. For example, a subject may be administered the AAV particles at 2, 3, 4, 5, or more than 5 sites.

In some embodiments, a subject may be administered the AAV particles described herein using sustained delivery over a period of minutes, hours, or days. The infusion rate may be changed depending on the subject, distribution, formulation, or another delivery parameter known to those in the art.

In some embodiments, if continuous delivery (continuous infusion) of the AAV particles is used, the continuous infusion may be for I hour. 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or more than 24 hours.

In some embodiments, the intracranial pressure may be evaluated prior to administration. The route, volume. AAV particle concentration, infusion duration and/or vector titer may be optimized based on the intracranial pressure of a subject.

In some embodiments, the AAV particles may be delivered by systemic delivery. In some embodiments, the systemic delivery may be by intravascular administration.

In some embodiments, the AAV particles may be delivered by injection into the CSF pathway. Non-limiting examples of delivery to the CSF pathway include intrathecal and intracerebroventricular administration.

In some embodiments, the AAV particles may be delivered by direct (intraparenchymal) injection into the substance of an organ, e.g., one or more regions of the brain.

In some embodiments, the AAV particles may be delivered by subpial injection into the spinal cord. For example, subjects may be placed into a spinal immobilization apparatus. A dorsal laminectomy may be performed to expose the spinal cord. Guiding tubes and XYZ manipulators may be used to assist catheter placement. Subpial catheters may be placed into the subpial space by advancing the catheter from the guiding tube and AAV particles may be injected through the catheter (Miyanohara et al., Mol Ther Methods Clin Dev. 2016; 3: 16046). In some cases, the AAV particles may be injected into the cervical subpial space. In some cases, the AAV particles may be injected into the thoracic subpial space.

In some embodiments, the AAV particles may be delivered by direct injection to the CNS of a subject. In some embodiments, direct injection is intracerebral injection, intraparenchymal injection, intrathecal injection, intra-cisterna magna injection, or any combination thereof. In some embodiments, direct injection to the CNS of a subject comprises convection enhanced delivery (CED). In some embodiments, administration comprises peripheral injection. In some embodiments, peripheral injection is intravenous injection.

In some embodiments, the AAV particles may be delivered to a subject in order to increase a STXBP1 protein level in the CNS (e.g., a cell or tissue of the CNS, e.g., the cortex, striatum, thalamus, cerebellum, brainstem, and/or spinal cord, and/or fluid (e.g., CSF and/or serum)) as compared to a baseline level in the subject.

In some embodiments, the AAV particles may be delivered to a subject in order to increase a STXBP1 protein level in the CNS (e.g., a cell or tissue of the CNS, e.g., the cortex, striatum, thalamus, cerebellum, and/or brainstem), and/or fluid (e.g., CSF and/or serum) of the subject by transducing cells in these CNS regions. Transduction may also be referred to as the number of cells that are positive for STXBP1 protein.

In some embodiments, delivery of AAV particles comprising a viral genome encoding STXBP1 as described herein to the CNS (e.g., a cell or tissue of the CNS, e.g., the cortex, stratum, thalamus, cerebellum, and/or brainstem), and/or fluid (e.g., CSF and/or serum) of the subject by transducing cells in these CNS regions may lead to an increased expression of STXBP1 protein in one or more of those regions. In some embodiments, the increased STXBP1 protein expression may lead to improved survival and/or function of various cell types in these CNS regions and/or improvement of at least one symptom of a STXBP1-related disorder, such as a STXBP1 encephalopathy, epileptic encephalopathy. Ohtahara syndrome, developmental encephalopathy, West syndrome, early myoclonic epileptic encephalopathy. Lennox-Gaustaut syndrome, autism (e.g., autism with STXBP1 mutations and optionally further mutations), Dravet syndrome (not caused by mutations in SCN1A), or Ret syndrome phenotype (not caused by mutation of MECP2 or CDKL5).

In some embodiments, the AAV particles may be delivered to a subject in order to establish widespread distribution of STXBP1 throughout the CNS, e.g., by administering the AAV particles to the thalamus of the subject. In some embodiments, the increased expression of STXBP1 protein may lead to a reduction in at least one symptom of a STXBP1-related disorder such as epilepsy, autistic features, ataxia, generalized tremors, reduced STXBP1 activity, accumulation of glucocerebroside and other glycolipids, e.g., within immune cells (e.g., macrophages), build-up of synuclein aggregates (e.g., Lewy bodies), developmental delay, progressive encephalopathy, progressive dementia, ataxia, myoclonus, oculomotor dysfunction, bulbar palsy, generalized weakness, trembling of a limb, depression, visual hallucinations, cognitive decline, dystonia, or a combination thereof.

### Administration

In some embodiments, the present disclosure provides methods comprising administering viral vectors in accordance with the disclosure to a subject in need thereof. Viral vector pharmaceutical, diagnostic, or prophylactic compositions thereof, may be administered to a subject using any amount and any route of administration effective for treating, or diagnosing a disease, disorder, and/or condition associated with decreased STXBP1 expression or STXBP1 deficiency. In some embodiments, the disease, disorder, and/or condition is a STXBP1-related disorder, such as a STXBP1-related neurodegenerative or neuromuscular disorder (e.g., STXBP1 encephalopathy, epileptic encephalopathy. Ohtahara syndrome, developmental encephalopathy, West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5)).

Compositions in accordance with the disclosure may be formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex, and diet of the patient; the time of administration, route of administration, and rate of excretion of the specific protein employed; the duration of the treatment; drugs used in combination or coincidental with the specific compound employed; and like factors well known in the medical arts.

In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of single unit dose or total daily dose into two or more doses, e.g., two or more administrations of the single unit dose. As used herein, a “single unit dose” is a dose of any therapeutic composition administered in one dose/at one time/single route/single point of contact, i.e., single administration event. In some embodiments, a single unit dose is provided as a discrete dosage from (e.g., a tablet, capsule, patch, loaded syringe, vial, etc.), As used herein, a “total daily dose” is an amount given or prescribed in 24-hour period. It may be administered as a single unit dose. The viral particles may be formulated in buffer only or in a formulation described herein.

In some embodiments, a pharmaceutical composition described herein can be formulated into a topical, intranasal, pulmonary, intratracheal, or injectable dosage form. In some embodiments, a pharmaceutical composition described herein can be formulated in a dosage form suitable for intravenous, intraocular, intravitreal, intramuscular, intracardiac, intraperitoneal, and/or subcutaneous administration.

In some embodiments, delivery of the AAV particles described herein results in minimal serious adverse events (SAEs) as a result of the delivery of the AAV particles.

### Combinations

The AAV particles may be used in combination with one or more other therapeutic, prophylactic, diagnostic, or imaging agents. The phrase “in combination with,” is not intended to require that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, and/or modify their distribution within the body.

The therapeutic agents may be approved by the US Food and Drug Administration or may be in clinical trial or at the preclinical research stage. The therapeutic agents may utilize any therapeutic modality known in the art, with non-limiting examples including gene silencing or intereference (i.e., miRNA, siRNA, RNAi, shRNA), gene editing (i.e., TALEN, CRISPR/Cas9 systems, zinc finger nucleases), and gene, protein, or enzyme replacement.

In some embodiments, an AAV particle described herein, or a pharmaceutical composition comprising the AAV particle, may be administered in combination with at least one additional therapeutic agent and/or therapy. In some embodiments, the at least one additional therapeutic agent and/or therapy comprises an agent and/or therapy for treating a STXBP1-related disorder such as a STXBP1-related neurodegenerative or neuromuscular disorder (e.g., STXBP1 encephalopathy, epileptic encephalopathy, Ohtahara syndrome, developmental encephalopathy. West syndrome, early myoclonic epileptic encephalopathy. Lennox-Gaustaut syndrome, autism (e.g., autism with STXBP1 mutations and optionally further mutations). Dravet syndrome (not caused by mutations in SCN1A), or Rett syndrome phenotype (not caused by mutation of MECP2 or CDKL5)).

In some embodiments, the at least one additional therapeutic agent and/or therapy comprises one or more anti-epileptic drugs (e.g., bromide, clobazam, felbamate, ganaxolone, lamotrigine, levetiracetam, phenobarbital, topiramate, valproate, or a combination thereof).

In some embodiments, the at least one additional therapeutic agent and/or therapy comprises an immunosuppressant. In some embodiments, the immunosuppressant may be administered to the subject prior to administration of an AAV particle or pharmaceutical composition described herein. In some embodiments, the immunosuppressant may be administered to the subject simultaneously with administration of an AAV particle or pharmaceutical composition described herein. In some embodiments, the immunosuppressant may be administered to the subject after administration of an AAV particle or pharmaceutical composition described herein. In some embodiments, the AAV particle or pharmaceutical composition is administered to a subject who is receiving or has received an immunosuppressant. In some embodiments, the immunosuppressant comprises a corticosteroid (for example, and without limitation, prednisone, prednisolone, methylprednisolone, and/or dexamethasone), rapanycin, mycophenolate mofetil, tacrolimus, rituximab, and/or eculizumab hydroxychloroquine. In some embodiments, the corticosteroid comprises prednisone, prednisolone, methylprednisolone, and/or dexamethasone. In some embodiments, the immunosuppressant comprises adrenocorticotropic hormone.

### Measurement of Expression

Expression of STXBP1 from viral genomes may be determined using various methods known in the art such as, but not limited to immunochemistry (e.g., IHC), enzyme-linked immunosorbent assay (ELISA), affinity ELISA, ELISPOT, flow cytometry, immunocytology, surface plasmon resonance analysis, kinetic exclusion assay, liquid chromatography-mass spectrometry (LCMS), high-performance liquid chromatography (HPLC), BCA assay, immunoelectrophoresis. Western blot, SDS-PAGE, protein immunoprecipitation. PCR, and/or in situ hybridization (ISH). In some embodiments, transgenes encoding STXBP1 delivered in different AAV capsid variants may have different expression levels in dorsal root ganglion (DRG).

In certain embodiments, the STXBP1 protein is detectable by an enzyme-linked immunosorbent assay (ELISA).

In certain embodiments, the STXBP1 protein is detectable by an immunohistochemistry assay.

In certain embodiments, the STXBP1 protein is detectable by Western blot.

In certain embodiments, expression of a STXBP1 gene, mRNA, and/or protein is measured in a cell or tissue of a subject who is receiving or has received an AAV particle described herein. In certain embodiments, the STXBP1 gene, mRNA, and/or protein expression is measured in a cell or tissue of the CNS (e.g., the cortex, striatum, thalamus, cerebellum, brainstem, and/or spinal cord) of the subject by transducing cells in these CNS regions. In certain embodiments, the STXBP1 gene, mRNA, and/or protein expression is measured in a peripheral cell or tissue, such as the heart, kidney, muscle (e.g., quadriceps muscle), pancreas, and/or liver.

### VII. Kits and Devices

### Kits

In some aspects, the present disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the vectors, constructs, or STXBP1 sequences (polypeptides or nucleotides) of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits of the disclosure may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and suitably aliquoted. Where there is more than one kit component. (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly used. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits of the disclosure. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, compounds and/or compositions of the present disclosure may be combined with, coated onto or embedded in a device. Devices may include, but are not limited to, dental implants, stents, bone replacements, artificial joints, valves, pacemakers and/or other implantable therapeutic device.

The present disclosure provides for devices which may incorporate viral vectors that encode one or more STXBP1 molecules. These devices contain in a stable formulation the viral vectors which may be immediately delivered to a subject in need thereof, such as a human patient.

Devices for administration may be employed to deliver the viral vectors encoding STXBP1 of the present disclosure according to single, multi- or split-dosing regimens taught herein.

Method and devices known in the art for multi-administration to cells, organs and tissues are contemplated for use in conjunction with the methods and compositions disclosed herein as embodiments of the present disclosure.

### VIII. Definitions

At various places in the present specification, substituents of compounds of the present disclosure are disclosed in groups or in ranges. It is specifically intended that the present disclosure include each and every individual sub-combination of the members of such groups and ranges. The following is a non-limiting list of term definitions.

Unless defined otherwise, all technical and scientific terms used herein have the same meaning as commonly understood by one of ordinary skill in the art to which the present invention pertains.

The articles “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

The term “comprising” is intended to be open and permits but does not require the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

Adeno-associated virus: As used herein, the term “adeno-associated virus” or “AAV” refers to members of the dependovirus genus or a functional variant thereof. Unless stated otherwise. “AAV” may refer to wildtype (i.e., naturally occurring) AAV or recombinant AAV.

AAV Particle: As used herein, an “AAV particle” refers to a particle comprising an AAV capsid, e.g., an AAV capsid variant (such as a parent capsid sequence with at least one peptide insert and/or with at least one substitution), and a polynucleotide, e.g., a viral genome or a vector genome. The AAV particle may be capable of delivering a STXBP1 polynucleotide to cells. The cells may be mammalian cells, e.g., human cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. In some embodiments, the AAV particle may comprise a peptide present in, e.g., inserted into, the capsid to enhance tropism for a desired target tissue. It is to be understood that reference to the AAV particle of the disclosure also includes pharmaceutical compositions thereof, even if not explicitly recited.

Administering: As used herein, the term “administering” refers to providing a pharmaceutical agent or composition to a subject.

Amelioration: As used herein, the term “amelioration” or “ameliorating” refers to a lessening of severity of at least one indicator of a condition or disease. For example, in the context of a neurodegenerative disorder, amelioration includes the reduction or stabilization of neuron loss.

Approximately: As used herein, the term “approximately” or “about,” as applied to one or more values of interest, refers to a value that is similar to, i.e., within 10% of, a stated reference value.

Baseline: The term “baseline,” when used to describe a measurement in a subject receiving or about to receive a treatment, refers to a measurement made before starting the treatment.

Capsid: As used herein, the term “capsid” refers to the exterior, e.g., a protein shell, of a virus particle, e.g., an AAV particle, that is substantially (e.g., >50%, >60%, >70%, >80%, >90%, >95%, >99%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” For example, and without limitation, an AAV capsid variant may refer to at least a VP1 protein, a VP2 protein, or a VP3 protein (e.g., all of the VP1, VP2, and VP3 proteins forming the AAV capsid) as will be clear from context. In some embodiments, the AAV capsid variant described herein may comprise a peptide insertion and/or substitution (i.e., replacement). In some embodiments, the AAV capsid variant described herein has the ability to encapsulate a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Cis-Elements: As used herein, cis-elements or the synonymous term “cis-regulatory elements” refer to regions of non-coding DNA which regulate the transcription of nearby genes. The Latin prefix “cis” translates to “on this side.” Cis-elements are found in the vicinity of the gene, or genes, they regulate. Examples of cis-elements include a Kozak sequence, SV40 introns, or a portion of the backbone.

CNS structures: As used herein. “CNS structures” refers to structures of the central nervous system and sub-structures thereof. Non-limiting examples of structures in the spinal cord may include, ventral horn, dorsal horn, white matter, and nervous system pathways or nuclei within. Non-limiting examples of structures in the brain include, forebrain, midbrain, hindbrain, diencephalon, telencephalon, myelencephalon, metencephalon, mesencephalon, prosencephalon, rhombencephalon, cortices, frontal lobe, parietal lobe, temporal lobe, occipital lobe, cerebrum, thalamus, hypothalamus, tectum, tegmentum, cerebellum, pons, medulla, anygdala, hippocampus, basal ganglia, corpus callosum, pituitary gland, putamen, striatum, ventricles and sub-structures thereof.

CNS Cells: As used herein, “CNS cells” refers to cells of the central nervous system and sub-structures thereof. Non-limiting examples of CNS cells include neurons and sub-types thereof, glia, microglia, oligodendrocytes, ependymal cells, and astrocytes. Non-limiting examples of neurons include sensory neurons, motor neurons, interneurons, unipolar cells, bipolar cells, multipolar cells, pseudo unipolar cells, pyramidal cells, basket cells, stellate cells, Purkinje cells, Betz cells, amacrine cells, granule cell, ovoid cell, medium aspiny neurons, large aspiny neurons, GABAergic neurons, and/or glutamatergic neurons.

Codon optimization: As used herein, the term “codon optimization” refers to a process of changing codons of a given gene in such a manner that the polypeptide sequence encoded by the gene remains the same.

Complementary and substantially complementary: As used herein, the term “complementary” refers to the ability of polynucleotides to form base pairs with one another. Perfect complementarity or 100% complementarity refers to the situation in which each nucleotide unit of one polynucleotide strand can form a hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form a hydrogen bond with each other, the polynucleotide strands exhibit 10% complementarity. In the same example, if 18 base pairs on each strand can form hydrogen bonds with each other, the polynucleotide strands exhibit 90% complementarity. The term “complementary” as used herein can encompass fully complementary or partially (e.g., substantially) complementary. “Fully complementary”, “perfect complementarity”, or “100% complementarity” refers to the situation in which each nucleotide unit of one polynucleotide or oligonucleotide strand can base-pair with a nucleotide unit of a second polynucleotide or oligonucleotide strand. As used herein, the term “substantially complementary” means that >50% of the nucleotide units of a first polynucleotide strand can base pair with nucleotide units on a second polynucleotide strand. When used in the context of RNA silencing, “substantially complementary” refers to an siRNA that has a sequence (e.g., in the antisense strand) that is sufficient to bind the desired target mRNA and to trigger the RNA silencing of the target mRNA.

Conservative substitution: As used herein, a conservative substitution, as applied to an amino acid sequence, also referred to as a “conservative amino acid substitution,” is one in which the amino acid residue is replaced with an amino acid residue having similar biochemical properties. When used in reference to a nucleic acid sequence, the term “conservative substitution” refers to a nucleotide replacement that results in an amino acid residue having similar biochemical properties compared to a reference sequence. Families of amino acid residues having similar biochemical properties have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Corresponding to: As used herein, the phrase “corresponding to” in the context of an amino acid sequence refers to the location of an amino acid in a reference sequence or the equivalent position in a modified sequence when aligned. For example, an amino acid corresponding to position 5 of SEQ ID NO: 36 refers to the amino acid at the fifth position from the N-terminus in SEQ ID NO: 36 or the equivalent position in an aligned sequence. As used herein, an amino acid at a position corresponding to that in a designated sequence may also be referred to as an amino acid at a particular position, numbered according to the designated sequence. For instance, an amino acid corresponding to position 5 of SEQ ID NO: 36 may also be referred to as an amino acid at position 5, numbered according to SEQ ID NO: 36, relative to a reference sequence of SEQ ID NO: 36, or as numbered according to a sequence corresponding to SEQ ID NO: 36.

Derivative: As used herein, the term “derivative” refers to a composition (e.g., sequence, compound, formulation, etc.) that is derived from, or finds its basis in, a parent composition. Non-limiting examples of a parent composition include a wild-type or original amino acid or nucleic acid sequence, or an undiluted formulation. In some embodiments, a derivative is a variant of a parent composition. A derivative may differ from the parent composition by less than about ⅙, less than about 5%, less than about 10%, less than about 15%, less than about 20%, less than about 25%, less than about 30%, less than about 35%, less than about 40%, less than about 45%, or less than about 50%. In certain embodiments, a derivative may differ from a parent composition by more than about 50%. In certain embodiments, a derivative may differ from a parent composition by more than about 75%. In some embodiments, a derivative may be a fragment or truncation of a parent amino acid or nucleotide sequence. As a non-limiting example, a derivative may be a sequence with a nucleotide, amino acid, or peptide substitution and/or insertion as compared to a parent nucleic acid or amino acid sequence (e.g., as compared to AAV9).

Effective amount: As used herein, the term “effective amount” or “therapeutically effective amount” of an agent is that amount sufficient to effect beneficial or desired results. An effective amount is provided in a single dose or multiple doses to treat, improve symptoms of, delay progression of symptoms, diagnose, prevent, and/or delay the onset of the infection, disease, disorder, and/or condition.

Excipient: As used herein, the term “excipient” refers to an inactive substance that serves as the vehicle or medium for an active pharmaceutical agent or other active substance.

Formulation: As used herein, a “formulation” includes at least one active ingredient (e.g., an AAV particle) and at least one inactive ingredient (e.g., a pharmaceutically acceptable excipient).

Fragment: A “fragment,” as used herein, refers to a contiguous portion of a reference sequence. A fragment may comprise a functional fragment that retains at least one activity of the reference sequence. For example, fragments of proteins may comprise polypeptides obtained by digesting full-length protein isolated from cultured cells. A fragment may also refer to a truncation (e.g., an N-terminal and/or C-terminal truncation) of a protein or a truncation (e.g., at the 5′ and/or 3′ end) of a nucleic acid. A protein fragment may be obtained by expression of a truncated nucleic acid, such that the nucleic acid encodes a portion of the full-length protein.

Healthy individual: As used herein, the term “healthy individual” refers to an individual who does not have a disease or disorder associated with STXBP1 protein deficiency, e.g., an individual who does not have a STXBP1-related disorder.

Humanized: As used herein, the term “humanized” refers to a non-human sequence of a polynucleotide or a polypeptide which has been altered to increase its similarity to a corresponding human sequence.

Identity: As used herein, the term “identity” refers to the overall relatedness between polymeric molecules, e.g., between oligonucleotide molecules (e.g., DNA molecules and/or RNA molecules) and/or between polypeptide molecules. Calculation of the percent identity of two polynucleotide sequences, for example, may be performed by aligning the two sequences for optimal comparison purposes (e.g., gaps can be introduced in one or both of a first and a second nucleic acid sequences for optimal alignment and non-identical sequences can be disregarded for comparison purposes). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A, M., ed., Oxford University Press. New York, 1988: Biocomputing: Informatics and Genome Projects, Smith, D, W., ed., Academic Press. New York, 1993: Sequence Analysis in Molecular Biology, von Heinje, G., Academic Press, 1987. Computer Analysis of Sequence Data. Part I, Griffin. A, M., and Griffin. H, G., eds., Humana Press. New Jersey,1994; and Sequence Analysis Primer, Gribskov, M, and Devereux, J., eds., M Stockton Press, New York, 1991: each of which is incorporated herein by reference in its entirety. For example, the percent identity between two nucleotide sequences can be determined using the algorithm of Myers and Miller (CABIOS. 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna. CMP matrix. Methods commonly employed to determine percent identity between sequences include, but are not limited to those disclosed in Carillo, H., and Lipman. D., SIAM J Applied Math., 48:1073 (1988): incorporated herein by reference in its entirety. Techniques for determining identity are codified in publicly available computer programs. Computer software to determine homology between two sequences include, but are not limited to, GCG program package. Devereux. J., et al., Nucleic Acids Research, 12(1), 387 (1984)), the Basic Local Alignment Search Tool (BLAST, which includes, e.g., BLASTP for protein sequences and BLASTN for nucleic acid sequences), and FASTA Altschul. S, F, et al., J. Molecular Biol., 215, 403 (1990)). EMBOSS Needle, Clustal Omega, Benchling, and Geneious. In preferred embodiments, sequence identity may be determined using BLAST, Clustal Omega, or EMBOSS Needle.

Inverted terminal repeat: As used herein, the term “inverted terminal repeat” or “ITR” refers to a cis-regulatory element for the packaging of polynucleotide sequences into viral capsids.

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

miRNA binding site: As used herein, a “miRNA binding site” or “miR binding site” refers either to a DNA sequence corresponding to an RNA sequence that is bound by a microRNA, or to the RNA sequence that is bound by the microRNA. The miR binding site is capable of binding, or binds, in whole or in part to a microRNA (miRNA, miR) through complete or partial hybridization. A miR binding site may be encoded or transcribed in series, also referred to as a “miR binding site series” or “miR BSs”, which includes two or more miR binding sites having the same or a different nucleic acid sequence.

Modification: As used herein, the term “modification” or “modified.” refers to any substance, compound, or molecule that has been changed in any way. For example, a modification in an amino acid sequence may comprise a substitution (e.g., a conservative substitution), an insertion, and/or a deletion of one or more amino acids in the sequence.

Neurological disease: As used herein, a “neurological disease” is any disease associated with the central or peripheral nervous system and components thereof (e.g., neurons).

Operably linked: As used herein, the phrase “operably linked” refers to a functional connection between two or more molecules, constructs, transcripts, entities, moieties or the like.

Payload: As used herein, “payload.” “payload sequence.” or “payload region” refers to one or more polynucleotides or polynucleotide regions encoded by or within a viral genome or an expression product of such polynucleotide or polynucleotide region, e.g., a transgene, a polynucleotide encoding a polypeptide.

Payload construct: As used herein. “payload construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector. As used herein, “payload construct vector” is a vector encoding or comprising a payload construct, and regulatory regions for replication and expression in bacterial cells. The payload construct vector may also comprise a component for viral expression in a viral replication cell.

Pharmaceutically acceptable: The phrase “pharmaceutically acceptable” is employed herein to refer to those compounds, materials, compositions, and/or dosage forms which are suitable for use in contact with the tissues of human beings and animals.

Pharmaceutically acceptable excipients: As used herein, the term “pharmaceutically acceptable excipient,” as used herein, refers to any ingredient other than active agents (e.g., as described herein) present in pharmaceutical compositions that can function as vehicles for suspending and/or dissolving active agents.

Pharmaceutically acceptable salts: Pharmaceutically acceptable salts of the compounds described herein are forms of the disclosed compounds wherein the acid or base moiety is in its salt form (e.g., as generated by reacting a free base group with a suitable organic acid). Examples of pharmaceutically acceptable salts include, but are not limited to, mineral or organic acid salts of basic residues such as amines: alkali or organic salts of acidic residues such as carboxylic acids; and the like.

Pharmaceutical Composition: As used herein, the term “pharmaceutical composition” or pharmaceutically acceptable composition” comprises AAV polynucleotides, AAV genomes, or AAV particle and one or more pharmaceutically acceptable excipients, solvents, adjuvants, and/or the like.

Position: The term “position.” as used herein in the context of an amino acid sequence, refers to the location of a particular amino acid or set of amino acids relative to a larger sequence. A position or positions of amino acids may interchangeably be referred to by an amino acid number or numbers of a reference sequence. For example, and unless otherwise specified, “positions 1-742, as numbered according to SEQ ID NO: 982” is interchangeable with “amino acids 1-742, as numbered according to SEQ ID NO: 982.”

Preventing: As used herein, the term “preventing” refers to partially or completely delaying onset of an infection, disease, disorder and/or condition: partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition. The term “prevention” or “preventing” of an infection, disease, disorder and/or condition may be considered a subset within the meaning with the term “treatment” or “treating” of the infection, disease, disorder and/or condition.

Region: As used herein, the term “region” refers to a zone or general area. In some embodiments, when referring to a protein or protein module, a region may comprise a linear sequence of amino acids along the protein or protein module or may comprise a three-dimensional area. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may comprise the N- and/or C-termini as well as surrounding amino acids. When referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and/or 3′ terminal.

Sample: As used herein, the term “sample” or “biological sample” refers to a subset of tissues, cells, nucleic acids, or a component or part of the body (e.g., a body fluid, including but not limited to blood, mucus, lymphatic fluid, synovial fluid, cerebrospinal fluid, saliva, amniotic fluid, amniotic cord blood, urine, vaginal fluid, and semen).

Self-complementary AAV: As used herein, the term “self-complementary AAV” refers to an AAV comprising at least a protein capsid and a self-complementary viral genome.

Serotype: As used herein, the term “serotype” refers to distinct variations in a capsid of an AAV based on surface antigens which allow epidemiologic classifications of the AAVs at the sub-species level.

Signal Sequences: As used herein, the phrase “signal sequence” refers to a sequence which can direct the transport or localization.

Similarity: As used herein, the term “similarity” refers to the overall relatedness between polymeric molecules, e.g., between polynucleotide molecules (e.g., DNA molecules and/or RNA molecules) and/or between poly peptide molecules. Calculation of percent similarity of polymeric molecules to one another can be performed in the same manner as a calculation of percent identity, except that calculation of percent similarity takes into account conservative substitutions as is understood in the art.

Spacer: As used herein, a “spacer” is generally any selected nucleic acid sequence of, e.g., 1, 2, 3, 4, 5, 6, 7, 8, 9, or 10 nucleotides in length, which is located between two or more consecutive miR binding site sequences. In some embodiments, spacers may also be more than 10 nucleotides in length, e.g., 20, 30, 40, or 50 or more than 50 nucleotides.

STXBP1-related disorder: As used herein, an “STXBP1-related disorder” refers to a disease, disorder, or condition in which one or more symptoms is caused by or associated with a deficiency of syntaxin-binding protein-1 (STXBP1) in a subject.

Subject: As used herein, the term “subject” or “patient” refers to any organism to which a composition in accordance with the disclosure may be administered, e.g., for experimental, diagnostic, prophylactic, and/or therapeutic purposes. Similarly, “subject” or “patient” refers to an organism who may seek, who may require, who is receiving, or who will receive treatment or who is under care by a trained professional for a particular disease or condition. Typical subjects include animals (e.g., mammals such as mice, rats, rabbits, non-human primates, and humans), As used herein, a subject or patient may be susceptible to, suspected of having, or have a deficiency in STXBP1 protein, e.g., human STXBP1 protein, and, e.g., may be susceptible to, suspected of having, or have a STXBP1-related disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition may be characterized by one or more of the following: (1) a genetic mutation associated with development of the disease, disorder, and/or condition; (2) a genetic poly morphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition: (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Target Cells: As used herein, “target cells” refers to any one or more cells of interest. The cells may be found in vitro, in vivo, in situ or in the tissue or organ of an organism. The organism may be an animal, preferably a mammal, more preferably a human and most preferably a human patient.

Target Tissue: As used herein. “target tissue” refers to a tissue of interest that may be found in vitro, in situ, or as part of an animal, preferably a mammal, more preferably a human and most preferably a human patient.

Therapeutic Agent: The term “therapeutic agent” refers to any agent that, when administered to a subject, elicits a desired biological and/or pharmacological effect.

Therapeutically Effective Outcome: As used herein, the term “therapeutically effective outcome” means an outcome that is sufficient in a subject suffering from or susceptible to an infection, disease, disorder, and/or condition, to treat, improve symptoms of, delay progression of symptoms, diagnose, prevent, and/or delay the onset of the infection, disease, disorder, and/or condition.

Treating: As used herein, the term “treating” refers to partially or completely alleviating, ameliorating, improving, relieving, delaying onset of, inhibiting progression of, reducing severity of, reducing incidence of, and/or preventing one or more symptoms or features of a particular infection, disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild-type or native form of a biomolecule or entity. Molecules or entities may undergo a series of modifications whereby each modified product may serve as the “unmodified” starting molecule or entity for a subsequent modification.

Variant: The term “variant” refers to a polypeptide or polynucleotide that has an amino acid or a nucleotide sequence that has at least 90% (e.g., at least 90%, at least 91%, at least 92%, at least 93%, at least 94%, at least 95%, at least 96%, at least 97%, at least 98%, or at least 99%) sequence identity to a reference sequence. The variant may be a functional variant. As used herein, the term “functional variant” refers to a polypeptide variant or a polynucleotide variant that has at least one activity of the reference sequence.

Factor: As used herein, a “vector” is any molecule or moiety which transports, transduces, or otherwise acts as a carrier of a heterologous molecule. Vectors of the present disclosure may be produced recombinantly and may be based on and/or may comprise adeno-associated virus (AAV) parent or reference sequence(s).

Viral genome: As used herein, a “viral genome”, “vector genome”, or “VG” is a polynucleotide comprising at least one inverted terminal repeat (ITR) and at least one nucleic acid sequence encoding a payload. A viral genome encodes at least one copy of the payload.

### EXAMPLES

The present disclosure is further illustrated by the following non-limiting examples.

### Example 1. High-Throughout Screen of TRACER AAV Library in NHP and Mice

A TRACER based method as described in WO2020072683, WO 2021/202651, and WO2021230987, the contents of which are herein incorporated by reference in their entirety, was used to generate the AAV capsid variants described herein. An orthogonal evolution approach was combined with a high throughput screening by NGS. Briefly, the library of AAV capsid variants was generated using a sliding window approach, where 6 amino acid sequences were inserted into 8 different positions across loop IV of AAV9, including immediately subsequent to positions 453, 454. 455, 456, 457, 458, 459, and 460, relative to a reference sequence numbered according to SEQ ID NO: 138. The initial library was passed twice through non-human primates (NHP, 2-4 years of age). After the second passage (e.g., 28 days post injection into two NHPs), RNA was extracted from six brain regions. Following RNA recovery and RT-PCR amplification, a systematic NGS enrichment analysis was performed to calculate fold enrichment relative to an AAV9 wild-type control. Following these two passages, approximately 21195 variants were identified with an average fold change greater than wild-type. Of the 21195 variants. 1558 demonstrated a fold-change of greater than 6 compared to wild-type and were detected across all brain regions investigated. Of these 1558, approximately 1470 variants were selected for constructing a synthetic library and a third passage through two NHPs. Within the 1470 variants selected for further characterization and investigation, there was a relatively even distribution for each insertion position of the sliding window used to generate the initial library.

After creation of the synthetic library with the sub-selected variants, the synthetic library was screened (passage 3) in two NHPs (2-4 years of age) and two strains of mice, BALB/c (n=3, 6-8 weeks of age) and C57B11/6 mice (n=3, 6-8 weeks of age), in a first cross-species evolution screen. The animals were injected intravenously with the synthetic library. After a period in vivo, (e.g., 28-days) RNA was extracted from nervous tissue, e.g., brain, spinal cord, and DRG of the NHPs and the brains of mice. Following RNA recovery and RT-PCR amplification, a systematic NGS enrichment analysis was performed, and the peptides comprised within the variants were identified and the capsid enrichment ratio for each variant compared to the wild-type AAV9 control was calculated (fold enrichment relative to wild-type AAV9) (Table 16). Values above 1 indicate an increase in expression relative to AAV9. All animals were dosed intravenously at 2-3 VG/kg across the screen.

As shown in Table 16, approximately 700 variants demonstrated an increase in expression relative to AAV9, and several variants demonstrated a greater than 10-fold enrichment relative to AAV9 in the brain of NHPs. Further, the variants demonstrating the greatest fold enrichment in the brain also demonstrated the greatest fold enrichment in the spinal cord relative to AAV9 in NHPs. These variants also demonstrated dc-targeting in the DRG (data not shown). For instance, the variant comprising GSGSPHSKAQNQQT (SEQ ID NO: 200) demonstrated a 76.6 fold enrichment in the brain, a 29.4 fold enrichment in the spinal cord, and 0.4 fold enrichment in the DRG of NHPs relative to AAV9; and GHDSPHKSGQNQQT (SEQ ID NO: 201) demonstrated a 62.6 fold enrichment in the brain, a 15.6 fold enrichment in the spinal cord, and 0.0 fold enrichment in the DRG of NHPs relative to AAV9. Also, across the peptides comprised within the AAV capsid variants with the greatest fold-enrichment in the NHP brain relative wild-type AAV9, it was observed that each of these peptides comprised an SPH motif in the same position (e.g., immediately subsequent to position 455, relative to a reference sequence numbered according to the amino acid sequence of SEQ ID NO: 138), regardless of the insertion position within the variant capsid, as well as a positive amino acid (e.g., K or R) in one of the next three residues subsequent to the SPH motif.

Those variants with the greatest fold enrichment in the brains of NHPs also had the greatest fold enrichment in the brains of both mouse species. Also, when comparing the fold enrichment relative to wild-type for each variant between the two species of mice investigated (C57BU16 and BALB/c mice), they were highly correlated (R²=0.8591).

TABLE 16
NGS fold-enrichment of AAV capsid variants in NHPs and mice

| | | | | Fold | Fold |
|---|---|---|---|---|---|
| | | Fold | | enrichment | enrichment |
| | | enrichment | Fold enrichment | over | over |
| | SEQ | over | over A.AV9 | AAV9 in the | AAV9 in the |
| | ID | AAV9 in the | in the spinal | brain of Mice | brain of Mice |
| Peptide Sequence | NO: | brain of NHPs | cord of NHPs | (C57BI/6) | (BALB/c) |
| GSGSPHSKAQNQQT | 200 | 73.615 | 29.402 | 25.293 | 41.304 |
| GHDSPHKSGQNQQT | 201 | 62.612 | 15.641 | 63.993 | 49.760 |
| GSGSPHARMQNQQT | 202 | 56.138 | 22.690 | 7.795 | 4.164 |
| GSGSPHVKSQNQQT | 203 | 37.551 | 13.649 | 8.069 | 15.861 |
| GQDSPHKSGQNQQT | 204 | 24.569 | 3.548 | 57.344 | 42.615 |
| GSGSPHASRQNQQT | 205 | 18.265 | 7.804 | 28.028 | 36.577 |
| GSGSPHASRQNKQT | 206 | 17.520 | 35.029 | 13.096 | 18.114 |
| GSGSPHVKIQNQQT | 207 | 16.854 | 9.068 | 2.173 | 2.227 |
| GSGSPHSKAKNQQT | 208 | 14.458 | 0.049 | 21.494 | 23.556 |
| GSGSPHKKNQNQQT | 209 | 12.991 | 0.379 | 25.958 | 7.415 |
| GSGSPHVRMQNQQT | 210 | 11.574 | 6.764 | 9.121 | 10.076 |
| GSGSPHASRQKQQT | 211 | 11.417 | 0.005 | 7.413 | 12.400 |
| GHSSPHRSGQNQQT | 212 | 10.357 | 1.887 | 23.197 | 25.442 |
| GMRTYHLSGQNQQT | 213 | 9.241 | 1.939 | 2.033 | 1.586 |
| GSGSPHTRGQNQQT | 214 | 7.092 | 3.815 | 10.801 | 6.240 |
| GSGIIPVSSQNQQT | 215 | 6.352 | 0.000 | 0.642 | 0.253 |
| GSEYGHKSGQNQQT | 216 | 6.308 | 2.750 | 5.198 | 5.332 |
| GRGQNVSSVHRQQT | 217 | 5.404 | 0.000 | 1.206 | 0.691 |
| GSSHRFYGGQNQQT | 218 | 4.732 | 0.000 | 0.787 | 0.110 |
| GYFVAAWSGQNQQT | 219 | 4.488 | 0.000 | 0.071 | 0.175 |
| GSVLHSHAAQNQQT | 220 | 4.150 | 6.448 | 0.675 | 0.423 |
| GSGDLVVSTQNQQT | 221 | 3.874 | 1.177 | 0.411 | 0.273 |
| GSYGMAASGQNQQT | 222 | 3.817 | 10.052 | 1.274 | 0.829 |
| GLNHFGASGQNQQT | 223 | 3.802 | 3.188 | 0.774 | 0.579 |
| GSTGSHSAGQNQHT | 224 | 3.717 | 0.285 | 1.190 | 0.850 |
| GLAGHTVSGQNQQT | 225 | 3.632 | 0.229 | 0.972 | 0.202 |
| GIILGASSGQNQQT | 226 | 3.630 | 4.868 | 1.378 | 0.865 |
| GSGVSTYNIQNQQT | 227 | 3.609 | 2.912 | 0.769 | 0.520 |
| GSLVSVQTGQNQQT | 228 | 3.534 | 6.043 | 0.903 | 0.469 |
| GQSSPHRSGQNQQT | 229 | 3.496 | 2.142 | 12.352 | 19.366 |
| GREYGHKSGQNQQT | 230 | 3.453 | 0.000 | 1.422 | 0.959 |
| GHTLTLSSGQNQQT | 231 | 3.405 | 5.648 | 0.648 | 0.606 |
| GSITLIPSGQNQQT | 232 | 3.361 | 3.917 | 0.326 | 0.435 |
| GSNGFTALGQNQQT | 233 | 3.361 | 2.663 | 0.830 | 0.332 |
| GSGHSSHSVQNQQT | 234 | 3.339 | 3.318 | 0.942 | 0.424 |
| GSGIPQRSGKNQQT | 235 | 3.331 | 0.000 | 1.418 | 1.685 |
| GSGDTLHMLQNQQT | 236 | 3.317 | 1.174 | 0.393 | 0.482 |
| GERHTVLSGQNQQT | 237 | 3.289 | 3.008 | 1.027 | 0.607 |
| GSGMPQSHIQNQQT | 238 | 3.289 | 11.609 | 0.514 | 0.334 |
| GSGQLSGIGGNQQT | 239 | 3.266 | 0.287 | 0.993 | 0.626 |
| GSGQNRKPASFAQT | 240 | 3.204 | 0.000 | 0.892 | 1.061 |
| GSGSVSQLGQNQQT | 241 | 3.184 | 2.307 | 0.596 | 0.375 |
| GSDELGTHGQNQQT | 242 | 3.171 | 0.348 | 1.038 | 0.750 |
| GQIVQNPSGQNQQT | 243 | 3.133 | 0.406 | 1.446 | 0.635 |
| GSGTQIPSQQNQQT | 244 | 3.112 | 1.224 | 0.470 | 0.151 |
| GSGQNQQSAREGLT | 245 | 3.111 | 5.632 | 1.221 | 1.104 |
| GSGLGMSTGQNQQT | 246 | 3.110 | 5.499 | 0.458 | 0.660 |
| GSGLPVLSGQNQQT | 247 | 3.100 | 4.149 | 0.631 | 0.210 |
| GSGHSIRTDQNQQT | 248 | 3.074 | 15.600 | 0.229 | 0.148 |
| GSGQSVQTVVNQQT | 249 | 3.057 | 5.441 | 0.582 | 0.240 |
| GSGQNRAQSRFQQT | 250 | 3.043 | 0.000 | 0.619 | 1.788 |
| GGGDLGRSSQNQQT | 251 | 3.036 | 4.830 | 0.916 | 0.539 |
| GGGTKMDSGQNQQT | 252 | 3.034 | 0.000 | 0.733 | 0.297 |
| GSGSPHPSRQNQQT | 253 | 3.017 | 1.993 | 1.869 | 0.975 |
| GSGQFTNAGMNQQT | 254 | 2.969 | 0.936 | 0.565 | 0.418 |
| GGRNGHTVGQNQQT | 255 | 2.965 | 3.732 | 1.105 | 1.003 |
| GSGFGPQTGQNQQT | 256 | 2.964 | 2.861 | 1.280 | 0.849 |
| GRIDSHTSGQNQQT | 257 | 2.913 | 1.510 | 1.299 | 0.704 |
| GYEVIGSSGQNQQT | 258 | 2.891 | 0.000 | 2.459 | 0.319 |
| GSVHLSVTGQNQQT | 259 | 2.882 | 1.157 | 0.741 | 0.282 |
| GEMSYKGSGQNQQT | 260 | 2.865 | 0.209 | 1.808 | 0.569 |
| GNIAGSVSGQNQQT | 261 | 2.849 | 1.187 | 0.446 | 0.257 |
| GSGSHRDVSQNQQT | 262 | 2.843 | 4.022 | 0.626 | 0.550 |
| GGLGSMSSGQNQQT | 263 | 2.812 | 1.405 | 1.802 | 0.822 |
| GSGHLPQSAQNQQT | 264 | 2.803 | 7.828 | 0.826 | 0.496 |
| GGVLVGGSGQNQQT | 265 | 2.778 | 0.178 | 1.527 | 0.688 |
| GTHPYTSSGQNQQT | 266 | 2.775 | 1.684 | 0.758 | 0.471 |
| GSGQNQQLKENRST | 267 | 2.765 | 0.062 | 1.149 | 1.118 |
| GSGQNQQTSPHNHT | 268 | 2.761 | 3.132 | 1.524 | 0.845 |
| GSGTLYPQSQNQQT | 269 | 2.761 | 5.558 | 0.324 | 0.160 |
| GSGQNQQSNWITKT | 270 | 2.711 | 0.000 | 0.540 | 0.634 |
| GSGYTSLFLQNQQT | 271 | 2.710 | 0.010 | 0.490 | 1.044 |
| GSGVMTHVLQNQQT | 272 | 2.692 | 0.347 | 0.370 | 0.533 |
| GSVSDVRAGQNQQT | 273 | 2.661 | 1.647 | 0.267 | 0.747 |
| GSGQSHMATLNQQT | 274 | 2.657 | 0.724 | 1.173 | 0.504 |
| GSGLSVHLAQNQQT | 275 | 2.657 | 1.234 | 0.806 | 0.508 |
| GSGLSHATQQNQQT | 276 | 2.640 | 7.819 | 1.111 | 0.638 |
| GSGLSVQSGQNQQT | 277 | 2.637 | 2.929 | 1.695 | 1.005 |
| GSGHMTYREKNQQT | 278 | 2.633 | 5.267 | 1.257 | 0.540 |
| GSKGVPTPGQNQQT | 279 | 2.625 | 1.292 | 1.452 | 0.459 |
| GSGLLPLSSQNQQT | 280 | 2.612 | 1.130 | 0.501 | 0.293 |
| GNGLYAVSGQNQQT | 281 | 2.611 | 9.148 | 0.322 | 0.213 |
| GENGSPSSGQNQQT | 282 | 2.609 | 12.197 | 2.338 | 0.924 |
| GSGQIRHSDQNQQT | 283 | 2.600 | 12.884 | 1.170 | 0.320 |
| GGQVAPSSGQNQQT | 284 | 2.581 | 2.427 | 1.433 | 0.709 |
| GSFSMHTHGQNQQT | 285 | 2.535 | 0.118 | 1.027 | 0.693 |
| GSGQNQQVIQGSNT | 286 | 2.521 | 8.778 | 0.935 | 0.810 |
| GRVLHSHAGQNQQT | 287 | 2.513 | 0.826 | 1.294 | 0.908 |
| GSGQNQQTSLQDQT | 288 | 2.505 | 0.500 | 0.315 | 0.968 |
| GSGLGRAPVQNQQT | 289 | 2.503 | 2.214 | 0.841 | 0.383 |
| GNGESSASGQNQQT | 290 | 2.493 | 0.772 | 0.240 | 0.182 |
| GSGQMASRESNQQT | 291 | 2.492 | 0.300 | 0.341 | 0.288 |
| GPGLPNHSGQNQQT | 292 | 2.486 | 1.992 | 1.197 | 0.659 |
| GNIQWQGSGQNQQT | 293 | 2.468 | 6.266 | 1.182 | 0.837 |
| GMSAHMSSGQNQQT | 294 | 2.456 | 5.255 | 1.310 | 0.947 |
| GHSFVNRSGQNQQT | 295 | 2.447 | 11.148 | 1.305 | 0.756 |
| GRAVMDHSGQNQQT | 296 | 2.408 | 3.209 | 0.728 | 0.283 |
| GALTVMQSGQNQQT | 297 | 2.381 | 0.430 | 0.246 | 0.199 |
| GSGQRSPVLPNQQT | 298 | 2.369 | 6.230 | 0.434 | 0.526 |
| GSGQNGHLSLKQQT | 299 | 2.362 | 1.896 | 0.718 | 0.270 |
| GSLPRGTSDQNQQT | 300 | 2.362 | 0.000 | 0.453 | 0.495 |
| GVAGSLVSGQNQQT | 301 | 2.358 | 7.670 | 1.321 | 1.160 |
| GRGGIPQSGQNQQT | 302 | 2.352 | 8.683 | 1.639 | 1.181 |
| GSGQYASSIPNQQT | 303 | 2.346 | 3.321 | 1.022 | 0.489 |
| GTDEGRQSSQNQQT | 304 | 2.346 | 3.196 | 1.021 | 0.797 |
| GIFMQTPSGQNQQT | 305 | 2.344 | 6.198 | 0.938 | 0.252 |
| GSGQNQQTRLVDLT | 306 | 2.342 | 9.348 | 1.268 | 0.490 |
| GTREMPLSGQNQQT | 307 | 2.339 | 2.830 | 1.436 | 0.538 |
| GSRLVHVHGQNQQT | 308 | 2.334 | 1.174 | 1.277 | 0.934 |
| GSGRLVPNGPNQQT | 309 | 2.314 | 3.925 | 0.639 | 0.411 |
| GSGYLRESPQNQQT | 310 | 2.311 | 0.878 | 0.331 | 0.677 |
| GARIQNASGQKQQT | 311 | 2.300 | 2.103 | 1.220 | 1.039 |
| GLSNPMPSGQNQQT | 312 | 2.280 | 6.033 | 1.190 | 0.829 |
| GSTVQDTRGQNQQT | 313 | 2.270 | 4.979 | 0.576 | 0.473 |
| GPFGMPSSGQNQQT | 314 | 2.260 | 2.700 | 0.727 | 0.560 |
| GSGQNHGVLSNQQT | 315 | 2.254 | 1.603 | 1.113 | 0.701 |
| GSGYSMSQAQNQQT | 316 | 2.250 | 4.479 | 0.519 | 0.329 |
| GSGMLTHTLQNQQT | 317 | 2.246 | 2.272 | 0.496 | 0.199 |
| GRGSPHASRQNQQT | 318 | 2.241 | 0.000 | 5.050 | 5.856 |
| GLSWPSTSGQNQQT | 319 | 2.238 | 0.000 | 0.910 | 0.610 |
| GNSMERTSGQNQQT | 320 | 2.221 | 4.177 | 1.047 | 0.935 |
| GSGMSPSTLQNQQT | 321 | 2.216 | 3.053 | 0.318 | 0.153 |
| GSGHGQVLSQNQQT | 322 | 2.213 | 12.133 | 1.880 | 0.661 |
| GRGQIYSTGGNQQT | 323 | 2.210 | 11.629 | 1.329 | 0.743 |
| GVVAAHNSGQNQQT | 324 | 2.202 | 1.301 | 1.196 | 1.336 |
| GDSSTRHSGQNQQT | 325 | 2.194 | 0.000 | 0.662 | 0.412 |
| GSLVSQGAGQNQQT | 326 | 2.188 | 4.414 | 1.436 | 1.246 |
| GSLLQAHSGQNQQT | 327 | 2.182 | 1.008 | 0.575 | 0.748 |
| GSGHIYVGIQNQQT | 328 | 2.178 | 6.428 | 0.989 | 0.337 |
| GHHTTVQSGQNQQT | 329 | 2.177 | 6.245 | 0.851 | 0.755 |
| GSRQSKRNELNQQT | 330 | 2.177 | 0.000 | 1.325 | 0.232 |
| GSGQNQQHVSSPRT | 331 | 2.176 | 1.279 | 1.847 | 0.938 |
| GSSKELLWGQNQQT | 332 | 2.163 | 0.000 | 0.506 | 0.883 |
| GSLSTPSSGQNQQT | 333 | 2.159 | 1.279 | 1.094 | 0.669 |
| GSIGYAGQGQNQQT | 334 | 2.157 | 4.951 | 1.604 | 0.712 |
| GSGQNQRVSNSQQT | 335 | 2.146 | 0.492 | 1.086 | 0.985 |
| GSGYASHVQQNQQT | 336 | 2.146 | 3.038 | 1.157 | 0.758 |
| GSGEYSRSGQNQQT | 337 | 2.145 | 0.745 | 0.617 | 0.205 |
| GSVSTHSSGQNQQT | 338 | 2.145 | 3.446 | 1.198 | 0.918 |
| GSGQNQHSLGNYQT | 339 | 2.143 | 1.896 | 1.077 | 0.606 |
| GSGGLDTRGQNQQT | 340 | 2.139 | 6.216 | 0.236 | 0.197 |
| GNILHATSGQNQQT | 341 | 2.136 | 0.125 | 1.159 | 0.424 |
| GSGQSYIMTQNQQT | 342 | 2.136 | 6.755 | 0.297 | 0.231 |
| GSGQNQHSAPNSQT | 343 | 2.134 | 4.143 | 1.187 | 0.731 |
| GSGQNQQTMDHNRT | 344 | 2.130 | 4.944 | 0.642 | 0.440 |
| GSNGGVGIGQNQQT | 345 | 2.130 | 0.788 | 1.191 | 1.087 |
| GAGSIIPSGQNQQT | 346 | 2.129 | 7.164 | 0.595 | 0.249 |
| GSGQTHGGQHNQQT | 347 | 2.125 | 12.251 | 1.448 | 1.098 |
| GSNLSFQSGQNQQT | 348 | 2.122 | 5.853 | 1.087 | 0.719 |
| GATLQVHSGQNQQT | 349 | 2.122 | 2.219 | 0.623 | 0.545 |
| GSGFNQRSEQNQQT | 350 | 2.121 | 4.491 | 1.770 | 0.758 |
| GSGSLRDEDQNQQT | 351 | 2.120 | 6.846 | 0.586 | 0.272 |
| GSGDSITGKQNQQT | 352 | 2.112 | 1.295 | 0.793 | 0.306 |
| GSGQDRNIVQNQQT | 353 | 2.112 | 0.229 | 0.454 | 0.632 |
| GSGLSHSHQQNQQT | 354 | 2.109 | 5.852 | 1.256 | 0.592 |
| GSGQNQQTGMSSVK | 355 | 2.109 | 4.544 | 1.451 | 0.679 |
| GSVTHGISGQNQQT | 356 | 2.105 | 4.542 | 1.135 | 0.789 |
| GVVAHQPSGQNQQT | 357 | 2.103 | 0.152 | 0.910 | 2.267 |
| GSGPILGQLQNQQT | 358 | 2.097 | 2.058 | 0.470 | 0.123 |
| GSGHVPNSGLNQQT | 359 | 2.091 | 0.653 | 1.636 | 1.154 |
| GDAGVRSSGQNQQT | 360 | 2.068 | 3.918 | 1.033 | 1.193 |
| GSGSQLMSLQNQQT | 361 | 2.065 | 3.559 | 0.563 | 0.172 |
| GSGLDYSQRQNQQT | 362 | 2.056 | 0.837 | 0.484 | 0.217 |
| GSGQSSGRLINKQT | 363 | 2.055 | 28.135 | 0.543 | 0.277 |
| GSSVSPSSGQNQQT | 364 | 2.054 | 0.579 | 1.064 | 0.787 |
| GSGQVVGLSGNQQT | 365 | 2.052 | 7.212 | 0.785 | 0.881 |
| GSNMGVPLGQNQQT | 366 | 2.049 | 2.448 | 0.334 | 0.420 |
| GSFYPSSTGQNQQT | 367 | 2.047 | 2.374 | 0.420 | 0.277 |
| GSGQNQQTRLTDLT | 368 | 2.046 | 8.470 | 0.910 | 0.776 |
| GPTNGRSSGQNQQT | 369 | 2.034 | 8.903 | 0.936 | 1.308 |
| GSGLLHGKLQNQQT | 370 | 2.032 | 2.521 | 1.068 | 0.917 |
| GANMGHVSGQNQQT | 371 | 2.020 | 0.810 | 1.302 | 1.138 |
| GSGQNQQSGRGDLT | 372 | 2.019 | 6.919 | 0.524 | 1.152 |
| GSHGHYASGQKQQT | 373 | 2.016 | 0.000 | 0.895 | 0.685 |
| GSGDLRISPQNQQT | 374 | 2.012 | 16.207 | 0.620 | 0.237 |
| GSGMPVILGQNQQT | 375 | 2.005 | 0.150 | 0.840 | 0.287 |
| GRGVITSSGQNHQT | 376 | 2.004 | 0.864 | 1.656 | 0.669 |
| GSGHSVSGPQNQQT | 377 | 1.993 | 6.259 | 1.370 | 0.619 |
| GSRNGHIVGRNQQT | 378 | 1.993 | 0.000 | 1.162 | 0.367 |
| GAGVHMVSGQNQQT | 379 | 1.987 | 6.488 | 1.055 | 0.791 |
| GSGQNHRPSVLQQT | 380 | 1.983 | 5.582 | 0.433 | 0.582 |
| GSGSPRDSIQNQQT | 381 | 1.981 | 4.914 | 0.171 | 0.446 |
| GSGQGIHSSVNQQT | 382 | 1.981 | 4.873 | 0.632 | 0.634 |
| GSGQQLSITPNQQT | 383 | 1.979 | 10.280 | 0.845 | 0.201 |
| GGYHSQTSGQNQQT | 384 | 1.978 | 2.642 | 1.740 | 1.525 |
| GSLHHDNHGQNQQT | 385 | 1.976 | 0.980 | 0.968 | 0.463 |
| GIMARDSSGQNQQT | 386 | 1.972 | 3.486 | 1.320 | 0.904 |
| GVVHITNSGQNQQT | 387 | 1.969 | 0.504 | 0.794 | 0.846 |
| GSGQNQHSAPFNQT | 388 | 1.969 | 0.499 | 0.759 | 0.870 |
| GSGQTSGLKQNQQT | 389 | 1.968 | 3.927 | 0.394 | 0.334 |
| GSGQNQQTSLSNTA | 390 | 1.959 | 1.186 | 1.567 | 1.182 |
| GSGQNQAVHNKSQT | 391 | 1.956 | 3.791 | 1.465 | 1.083 |
| GVHTHLPSGQNQQT | 392 | 1.952 | 1.364 | 1.414 | 0.796 |
| GHLTMHNSGQNHQT | 393 | 1.938 | 1.798 | 1.030 | 0.586 |
| GSGSSSRPYQNQQT | 394 | 1.934 | 3.823 | 0.962 | 0.496 |
| GILLATPSGQNQQT | 395 | 1.931 | 8.205 | 1.341 | 0.288 |
| GSGQNAGSFPNQQT | 396 | 1.928 | 12.575 | 1.091 | 0.286 |
| GSRDGHTVGQNQQT | 397 | 1.928 | 7.089 | 0.495 | 0.661 |
| GSLLISTSGQNQQT | 398 | 1.919 | 5.763 | 1.488 | 0.808 |
| GSGAMPSHGQNQQT | 399 | 1.915 | 0.000 | 1.142 | 0.912 |
| GALVSPISGQNQQI | 400 | 1.912 | 1.051 | 0.640 | 0.347 |
| GSLSSHGVGQNQQT | 401 | 1.911 | 7.498 | 1.218 | 0.804 |
| GSGQNQQASLAMRT | 402 | 1.910 | 3.577 | 2.066 | 1.638 |
| GPGLGSHSGQNQQT | 403 | 1.906 | 14.563 | 0.880 | 1.195 |
| GHDSQHKSGQNQQT | 404 | 1.904 | 6.988 | 1.154 | 0.869 |
| GSGLTLSATQNQQT | 405 | 1.901 | 0.193 | 0.708 | 0.340 |
| GSGQVVAHVGNQQT | 406 | 1.901 | 0.833 | 0.800 | 0.321 |
| GSGLRTMTTQNQQT | 407 | 1.900 | 8.939 | 0.838 | 0.594 |
| GSGQVGRLLQNQQT | 408 | 1.899 | 1.762 | 0.773 | 0.748 |
| GSGQLSHQSVNQQT | 409 | 1.898 | 4.032 | 0.720 | 0.695 |
| GSGDRYQTLQNQQT | 410 | 1.897 | 1.075 | 0.645 | 0.318 |
| GSGQNQQLKSSAQT | 411 | 1.891 | 1.197 | 0.908 | 0.716 |
| GSGQNQYSIPVAQT | 412 | 1.891 | 1.194 | 0.511 | 0.297 |
| GSGERLHLTQNQQT | 413 | 1.885 | 1.456 | 0.387 | 0.245 |
| GSGHNQQVRTAPNT | 414 | 1.885 | 1.022 | 1.006 | 0.580 |
| GGLSHVMSGQNQQT | 415 | 1.875 | 0.403 | 0.885 | 0.378 |
| GSGQSHRDVLNQQT | 416 | 1.872 | 15.082 | 0.138 | 0.280 |
| GSGQNLAGRMDQQT | 417 | 1.864 | 0.085 | 0.362 | 0.295 |
| GSGQNQQTNRGNPM | 418 | 1.860 | 3.402 | 1.349 | 1.098 |
| GSGQSYQRDHNQQT | 419 | 1.859 | 8.013 | 0.779 | 0.323 |
| GSLLSAGMGQNQHT | 420 | 1.856 | 6.168 | 0.589 | 0.342 |
| GSGQNQQTATYRNI | 421 | 1.854 | 2.207 | 0.818 | 1.437 |
| GSGQNQQTSGTINC | 422 | 1.854 | 8.161 | 1.040 | 0.806 |
| GMTSHSVSGQNQQT | 423 | 1.850 | 2.732 | 0.220 | 0.154 |
| GSSQSTGYQPNQQT | 424 | 1.847 | 3.388 | 0.522 | 0.577 |
| GSLKPTTLGQNQQT | 425 | 1.840 | 0.476 | 0.175 | 0.220 |
| GRMFSLGSGQNQQT | 426 | 1.836 | 8.429 | 1.630 | 1.174 |
| GSGQNQQTALGVKC | 427 | 1.835 | 1.343 | 1.378 | 1.014 |
| GAMVSHSSGQNQQT | 428 | 1.833 | 8.999 | 0.739 | 0.868 |
| GSGQNQQRNSDSVT | 429 | 1.829 | 0.000 | 1.238 | 0.842 |
| GSGQSMTLHLNQQT | 430 | 1.827 | 0.991 | 0.721 | 0.248 |
| GSGQVHQAEVNQQT | 431 | 1.825 | 0.152 | 0.436 | 0.287 |
| GSGQNQSQNHLQQT | 432 | 1.825 | 0.600 | 1.063 | 0.772 |
| GSLLTTASGQNQQT | 433 | 1.822 | 0.780 | 0.938 | 0.635 |
| GSGLIRTAAQNQQT | 434 | 1.822 | 8.339 | 0.808 | 0.998 |
| GSGQNQQTVSRQST | 435 | 1.820 | 0.472 | 1.330 | 0.796 |
| GSGQYANHGINQQT | 436 | 1.820 | 5.717 | 0.906 | 0.701 |
| GSRSTGPSGQNQQT | 437 | 1.819 | 2.479 | 0.440 | 0.466 |
| GRGVQQKLQQNQQT | 438 | 1.817 | 0.000 | 1.974 | 0.823 |
| GSGQNQQVHLSTGT | 439 | 1.811 | 0.266 | 1.011 | 0.455 |
| GSGQNQQLSAKSST | 440 | 1.809 | 1.567 | 1.224 | 1.115 |
| GSGYKAARPQNQQT | 441 | 1.803 | 0.000 | 1.418 | 0.337 |
| GSAGISPSGQNQQT | 442 | 1.797 | 1.812 | 0.784 | 0.622 |
| GSGQNRAHAFLQQT | 443 | 1.795 | 0.000 | 1.200 | 1.271 |
| GSGLSGITMQNQQT | 444 | 1.792 | 14.796 | 0.862 | 0.496 |
| GPGSAHSSGQNQQT | 445 | 1.785 | 4.392 | 1.099 | 0.872 |
| GSSHTQALGQNQQT | 446 | 1.784 | 0.143 | 0.882 | 0.874 |
| GSGVHGVSSQNQQT | 447 | 1.781 | 4.519 | 1.504 | 0.951 |
| GSSGRDMGGQNQQT | 448 | 1.778 | 2.177 | 1.052 | 0.595 |
| GERAFPTSGQNQQT | 449 | 1.775 | 6.515 | 0.972 | 0.362 |
| GGRIVSLSGQNQQT | 450 | 1.766 | 4.936 | 1.161 | 0.847 |
| GSGQNSYSHTSQQT | 451 | 1.765 | 2.262 | 1.130 | 0.658 |
| GLGYPGSSGQNQQT | 452 | 1.763 | 7.090 | 0.929 | 0.577 |
| GSGPQSHTGQNQQT | 453 | 1.757 | 9.490 | 0.958 | 0.447 |
| GSGQNQQLSRDAST | 454 | 1.754 | 3.716 | 1.877 | 0.611 |
| GSGQILHSVPNQQT | 455 | 1.752 | 1.316 | 0.398 | 0.240 |
| GSGEHTDSRQNQQT | 456 | 1.748 | 4.384 | 7.344 | 0.575 |
| GSGQSHSLATNQQT | 457 | 1.745 | 2.711 | 1.021 | 0.343 |
| GSGQNQQTLSKPWT | 458 | 1.743 | 0.253 | 0.845 | 0.733 |
| GSGHAAISQQNQQT | 459 | 1.742 | 2.373 | 1.211 | 0.520 |
| GSGQNQQQIGGNST | 460 | 1.741 | 6.169 | 0.877 | 0.576 |
| GGGPMAGSGQNQQT | 461 | 1.735 | 2.815 | 1.049 | 0.372 |
| GMRMEYQSGQNQQT | 462 | 1.729 | 3.695 | 0.644 | 0.632 |
| GSGQNQQGTLLHQT | 463 | 1.728 | 2.065 | 1.347 | 1.303 |
| GSGQNQRSSGGVQT | 464 | 1.723 | 2.056 | 1.805 | 1.165 |
| GSGQNQRGALATQT | 465 | 1.722 | 1.117 | 0.899 | 0.891 |
| GSGTVHAATQNQQT | 466 | 1.721 | 1.676 | 0.563 | 0.476 |
| GSRMTQQFGQNQQT | 467 | 1.720 | 25.798 | 1.233 | 0.976 |
| GSSSPGASGQNQQT | 468 | 1.717 | 1.244 | 1.378 | 0.660 |
| GHPSPHVSGQNQQT | 469 | 1.713 | 0.416 | 0.551 | 0.488 |
| GSGSHHASRQNQQT | 470 | 1.712 | 0.451 | 3.073 | 0.584 |
| GAVGHSYSGQNQQT | 471 | 1.706 | 0.808 | 0.306 | 0.536 |
| GSRSQYDIGQNQQT | 472 | 1.706 | 0.112 | 0.528 | 0.193 |
| GSGQGPQERGNQQT | 473 | 1.702 | 1.269 | 0.846 | 0.313 |
| GSIAHVGIGQNQQT | 474 | 1.696 | 1.264 | 0.837 | 1.045 |
| GSGQNQQKQNHGNT | 475 | 1.695 | 5.349 | 1.538 | 1.340 |
| GSGQNQQALGSQRT | 476 | 1.695 | 1.934 | 1.419 | 0.562 |
| GSGAITHMPQNQQT | 477 | 1.695 | 1.681 | 0.647 | 0.411 |
| GSGQRNPLLLNQQT | 478 | 1.693 | 0.144 | 0.662 | 0.740 |
| GSSGIPVSHQNQQT | 479 | 1.690 | 3.384 | 0.820 | 0.333 |
| GVHSLIPSGQNQQT | 480 | 1.687 | 4.104 | 0.475 | 0.215 |
| GVIVLHGSGQNQQT | 481 | 1.682 | 14.166 | 1.074 | 1.098 |
| GGTRVVDSGQNQQT | 482 | 1.676 | 9.735 | 0.676 | 0.370 |
| GSGGVTYQSQNQQT | 483 | 1.673 | 7.283 | 0.649 | 0.181 |
| GSGQNQAGHGPGQT | 484 | 1.670 | 2.861 | 1.040 | 0.887 |
| GSGQLVTSGPNQQT | 485 | 1.669 | 5.271 | 0.964 | 0.433 |
| GSGIAAQRTQNQQT | 486 | 1.665 | 2.691 | 1.062 | 0.754 |
| GSTPAGVGGQNQQT | 487 | 1.663 | 2.733 | 0.593 | 0.477 |
| GSGQNQQTSTGVHS | 488 | 1.660 | 8.271 | 1.039 | 1.075 |
| GSGQIRQLVDNQQT | 489 | 1.657 | 5.529 | 0.314 | 0.272 |
| GSLIGMQSGQNQQT | 490 | 1.656 | 6.783 | 0.797 | 0.392 |
| GSGQIKGKMDNQQT | 491 | 1.654 | 2.601 | 1.065 | 1.012 |
| GSGSDMSSWQNQQT | 492 | 1.651 | 0.175 | 0.281 | 0.303 |
| GRGQNQQHTGLATT | 493 | 1.650 | 6.174 | 1.134 | 0.691 |
| GSGQNQQTLYSSNT | 494 | 1.642 | 1.044 | 0.664 | 0.368 |
| GSGQTQVLKSNQQT | 495 | 1.640 | 3.031 | 1.599 | 0.975 |
| GSRTLSNVGQNQQT | 496 | 1.640 | 3.219 | 0.617 | 0.542 |
| GSGVQHSLPQNQQT | 497 | 1.639 | 0.764 | 0.440 | 0.387 |
| GNYLHQASGQNQQT | 498 | 1.635 | 1.454 | 0.816 | 0.181 |
| GSGGTSVHQQNQQT | 499 | 1.629 | 0.000 | 0.585 | 0.195 |
| GMDHSRPSGQNQQT | 500 | 1.627 | 3.976 | 0.918 | 0.648 |
| GSGQNQQSMGTETT | 501 | 1.625 | 0.000 | 1.792 | 0.399 |
| GSGQNQQTPLRPPT | 502 | 1.624 | 0.352 | 0.874 | 0.472 |
| GSGQNQHHSVSQQT | 503 | 1.623 | 3.700 | 0.605 | 0.334 |
| GSGQLRSLSTNQQT | 504 | 1.622 | 6.855 | 1.310 | 0.382 |
| GSGSPRQLSQNQQT | 505 | 1.621 | 0.873 | 0.520 | 0.273 |
| GSGQNQQTTASSHT | 506 | 1.618 | 7.404 | 0.745 | 0.678 |
| GRGQVVSTHQNQQT | 507 | 1.607 | 3.318 | 0.931 | 0.561 |
| GSAQVSMVGQNQQT | 508 | 1.601 | 1.332 | 0.500 | 0.285 |
| GSSTLVTIGKNQQT | 509 | 1.592 | 4.316 | 0.917 | 0.819 |
| GEAHQASSGQNQQT | 510 | 1.587 | 1.852 | 1.638 | 1.080 |
| GSGQPVLSISNQQT | 511 | 1.586 | 2.695 | 0.390 | 0.282 |
| GSGQSHRSELNQQT | 512 | 1.585 | 11.974 | 0.668 | 0.256 |
| GSSVGSPIGQNQQT | 513 | 1.584 | 3.574 | 1.059 | 0.706 |
| GSGMPIRNVQNQQT | 514 | 1.584 | 0.138 | 0.684 | 0.631 |
| GSSTRVDSGQNQQT | 515 | 1.584 | 2.774 | 0.704 | 0.660 |
| GSGQNQQTAMRSTI | 516 | 1.581 | 2.588 | 0.656 | 0.665 |
| GSGQNQQHSSSHLT | 517 | 1.581 | 2.782 | 1.091 | 0.859 |
| GSRNGHAVGQNQQT | 518 | 1.574 | 2.688 | 0.434 | 0.939 |
| GLGAYQSSGQNQQT | 519 | 1.574 | 0.696 | 1.407 | 0.688 |
| GPGLSGHSGQNQQT | 520 | 1.571 | 1.603 | 1.154 | 1.297 |
| GSTGIVSSGQNQQT | 521 | 1.570 | 0.927 | 2.141 | 1.046 |
| GSRTTQVIGQNQQT | 522 | 1.570 | 1.838 | 0.773 | 0.564 |
| GSGLLHRAQQNQQT | 523 | 1.569 | 0.724 | 1.583 | 0.646 |
| GSGQNAQQAAAQQT | 524 | 1.568 | 4.239 | 0.937 | 0.604 |
| GSGQNQQSALRTQT | 525 | 1.568 | 1.913 | 1.581 | 1.421 |
| GSGFLSDTRQNQQT | 526 | 1.566 | 45.953 | 0.473 | 0.575 |
| GSGLLYHDQQNQQT | 527 | 1.565 | 2.760 | 0.405 | 0.107 |
| GSGQNQHYSLHKQT | 528 | 1.563 | 3.399 | 1.485 | 1.273 |
| GSGHSPLPQQNQQT | 529 | 1.562 | 0.556 | 0.387 | 0.247 |
| GNGHSMRPNQNQQT | 530 | 1.560 | 2.341 | 0.693 | 0.376 |
| GSGLKWSTLQNQQT | 531 | 1.556 | 0.000 | 1.134 | 2.442 |
| GSGQMGRQAVNQQT | 532 | 1.554 | 1.529 | 0.535 | 0.411 |
| GSGQNQQTSGVLTL | 533 | 1.553 | 0.000 | 1.104 | 0.782 |
| GSGQNQQALHNPHI | 534 | 1.553 | 0.664 | 0.638 | 0.213 |
| GSGQNQQVIPNSKT | 535 | 1.548 | 1.036 | 0.844 | 0.376 |
| GSPLQDRVGQNQQT | 536 | 1.548 | 0.753 | 0.469 | 0.391 |
| GSGQNQYSSTNPQT | 537 | 1.542 | 2.251 | 0.544 | 0.535 |
| GAMTVTISGQNQQT | 538 | 1.542 | 6.249 | 0.443 | 0.257 |
| GSGQNQQLQTLIRT | 539 | 1.538 | 1.425 | 0.813 | 0.514 |
| GSGLRQTSQQNQQT | 540 | 1.537 | 2.067 | 0.978 | 0.705 |
| GSGQNQQTGLRQQT | 541 | 1.533 | 2.120 | 1.217 | 1.103 |
| GSGQTRQMKDNQQT | 542 | 1.530 | 11.079 | 0.841 | 0.214 |
| GSGQNHGLQSGQQT | 543 | 1.530 | 4.960 | 0.938 | 0.779 |
| GSGQSHRQPENQQT | 544 | 1.529 | 2.153 | 0.209 | 0.159 |
| GSGQDRHIVQNQQT | 545 | 1.527 | 11.068 | 0.285 | 0.162 |
| GSGQNQQLPHSNLT | 546 | 1.521 | 1.838 | 0.442 | 0.283 |
| GSGQLSVPYDNQQT | 547 | 1.521 | 0.000 | 0.622 | 0.111 |
| GSGRNPQTQPLQQT | 548 | 1.519 | 0.040 | 0.733 | 0.573 |
| GSGQPYSTGLNQQT | 549 | 1.519 | 1.403 | 0.612 | 0.376 |
| GSGQNQQTHGGLRD | 550 | 1.519 | 6.487 | 1.913 | 1.298 |
| GAYGMVSSGQNQQT | 551 | 1.518 | 3.469 | 0.732 | 0.773 |
| GSGIQSSYSQNQQT | 552 | 1.517 | 15.978 | 1.032 | 0.684 |
| GPRLSDQSGQNQQT | 553 | 1.511 | 0.364 | 0.640 | 0.579 |
| GSGQNQQTHPSPCT | 554 | 1.510 | 1.003 | 1.120 | 0.546 |
| GSGQSFQMHINQQT | 555 | 1.504 | 9.770 | 0.503 | 0.325 |
| GSGQNQQTGNPKHT | 556 | 1.504 | 5.973 | 1.391 | 1.139 |
| GFSSAVHSGQNQQT | 557 | 1.502 | 1.234 | 0.218 | 0.210 |
| GSCQNQQTSMSNAT | 558 | 1.501 | 6.766 | 1.605 | 0.745 |
| GSGQDMKQHHNQQT | 559 | 1.501 | 1.638 | 0.358 | 0.239 |
| GIRLSTPSGQNQQT | 560 | 1.498 | 4.334 | 0.804 | 0.522 |
| GSGQNQQTSVYMNT | 561 | 1.498 | 0.613 | 0.640 | 0.983 |
| GSGQNQYSQSSMQT | 562 | 1.494 | 4.278 | 0.375 | 0.309 |
| GSGQNQQSMADHTT | 563 | 1.494 | 1.728 | 0.428 | 0.215 |
| GWERSFVSGQNQQT | 564 | 1.492 | 0.943 | 0.490 | 0.538 |
| GLLAGKSSGQNQQT | 565 | 1.491 | 2.981 | 0.999 | 0.946 |
| GKSFVPQSGQNQQT | 566 | 1.489 | 2.502 | 1.798 | 0.430 |
| GSGQMQSAGSNQQT | 567 | 1.482 | 0.116 | 1.034 | 1.128 |
| GSDQNQRLTSSMQT | 568 | 1.479 | 0.164 | 0.875 | 0.670 |
| GESRAVISGQNQQT | 569 | 1.476 | 0.938 | 0.789 | 0.368 |
| GSVFGVPSGQNQQT | 570 | 1.474 | 1.248 | 0.685 | 0.213 |
| GSGLPDRNLQNQQT | 571 | 1.471 | 7.306 | 1.136 | 0.611 |
| GSGTHNSAIQNQQT | 572 | 1.469 | 0.570 | 0.762 | 0.574 |
| GSGMIIASMQNQQT | 573 | 1.469 | 6.722 | 1.135 | 0.415 |
| GGITWIDSGQNQQT | 574 | 1.462 | 4.535 | 1.472 | 0.468 |
| GSGQNQQASGRQQT | 575 | 1.458 | 3.179 | 0.943 | 0.991 |
| GSGQNQQPHLKSLT | 576 | 1.457 | 5.016 | 1.096 | 0.740 |
| GPPQHMTSGQNQQT | 577 | 1.457 | 1.547 | 0.509 | 0.677 |
| GSGQNQQASLPSRT | 578 | 1.456 | 0.389 | 0.930 | 0.673 |
| GSGQIVSTQTNQQT | 579 | 1.456 | 1.103 | 0.453 | 0.512 |
| GSGKGHSAGQNQQT | 580 | 1.453 | 0.936 | 1.035 | 1.173 |
| GSGQNTRLQLGQQT | 581 | 1.452 | 1.747 | 0.181 | 0.234 |
| GSVGSRPVGQNQQT | 582 | 1.442 | 11.363 | 1.182 | 0.716 |
| GSSHTLALGQNQQT | 583 | 1.441 | 7.071 | 0.851 | 0.406 |
| GMYEYSQSGQNQQT | 584 | 1.438 | 0.000 | 1.410 | 0.448 |
| GNGQNQQHSILHGT | 585 | 1.435 | 0.000 | 0.777 | 0.415 |
| GSGYNQPHLQNQQT | 586 | 1.435 | 4.512 | 0.711 | 0.395 |
| GPLVNASSGQNQQT | 587 | 1.434 | 5.239 | 0.831 | 0.343 |
| GSGQNQQVLTTART | 588 | 1.434 | 4.142 | 1.071 | 0.948 |
| GSGQNQHSVHNDQT | 589 | 1.428 | 0.000 | 0.521 | 0.515 |
| GAGLIMHSGQNQQT | 590 | 1.425 | 1.408 | 0.565 | 0.511 |
| GMGRHSASGQNQQT | 591 | 1.417 | 6.500 | 0.470 | 0.389 |
| GSHSQSGHGQNQQT | 592 | 1.413 | 1.240 | 0.696 | 0.318 |
| GSSTTIVSGQNHQT | 593 | 1.411 | 0.000 | 0.993 | 0.672 |
| GRHLVTASGQNQQT | 594 | 1.411 | 2.885 | 0.648 | 0.404 |
| GSGQNQQHANLNQT | 595 | 1.410 | 0.094 | 0.416 | 0.544 |
| GSGSTHSKAQNQQT | 596 | 1.410 | 0.515 | 0.921 | 0.801 |
| GSGQNKQMLSGNTT | 597 | 1.410 | 2.219 | 1.074 | 0.404 |
| GSGQVHNPTQNQQT | 598 | 1.410 | 2.488 | 1.021 | 0.542 |
| GSGQNQQIPHVHQT | 599 | 1.409 | 0.768 | 0.576 | 0.218 |
| GSLHAGLSGQNQQT | 600 | 1.408 | 1.739 | 1.286 | 0.936 |
| GPAQHGTSGQNQQT | 601 | 1.407 | 0.866 | 1.030 | 0.615 |
| GEKAVTSSGQNQQT | 602 | 1.402 | 0.998 | 0.558 | 0.327 |
| GSGQNQQTMANGQR | 603 | 1.394 | 0.216 | 1.169 | 1.230 |
| GSGSPHSKDQNQQT | 604 | 1.394 | 0.000 | 2.041 | 4.680 |
| GSFSMGYGGQNQQT | 605 | 1.393 | 18.476 | 1.908 | 1.030 |
| GSGTHLVSLQNQQT | 606 | 1.392 | 0.000 | 0.715 | 1.167 |
| GSGQMQPHVQNQQT | 607 | 1.389 | 9.381 | 0.387 | 0.153 |
| GSGQNQQVAGLNNT | 608 | 1.386 | 3.218 | 0.449 | 0.492 |
| GSSQNQQHDMRLRT | 609 | 1.386 | 2.645 | 0.669 | 0.552 |
| GPASLPISGQNQQT | 610 | 1.386 | 9.008 | 0.312 | 0.155 |
| GSGQNQQPPLATRT | 611 | 1.386 | 2.295 | 0.593 | 0.287 |
| GSSRVPVSGQNQQT | 612 | 1.385 | 13.191 | 0.870 | 0.485 |
| GSGQNQQTNIGHTT | 613 | 1.383 | 1.523 | 1.343 | 1.281 |
| GSGQNQQLVSRVQT | 614 | 1.381 | 1.195 | 0.656 | 0.466 |
| GPNSYPVSGQKQQT | 615 | 1.381 | 4.040 | 0.736 | 0.834 |
| GHAHYQASGQNQQT | 616 | 1.377 | 7.299 | 0.803 | 0.745 |
| GSGQALLSTGNQQT | 617 | 1.377 | 0.847 | 0.536 | 0.370 |
| GSGQLPRQMTNQQT | 618 | 1.376 | 3.550 | 0.400 | 0.562 |
| GSGFPKSTEQNQQT | 619 | 1.376 | 2.058 | 0.610 | 0.194 |
| GSRETSLSGQNQQT | 620 | 1.373 | 5.193 | 1.364 | 0.203 |
| GSGQNQQGTGVSHT | 621 | 1.371 | 4.295 | 1.417 | 0.749 |
| GSRIVPVYGQNQQT | 622 | 1.371 | 0.363 | 1.226 | 0.969 |
| GSNAQSAHGQNQQT | 623 | 1.371 | 0.888 | 0.976 | 0.245 |
| GAFHLAASGQNQQT | 624 | 1.369 | 18.165 | 0.994 | 0.775 |
| GSGQYRSSSDNQQT | 625 | 1.369 | 6.209 | 0.681 | 0.409 |
| GSGQVYISTPNQQT | 626 | 1.367 | 0.000 | 0.859 | 0.282 |
| GSGVSTQLLQNQQT | 627 | 1.367 | 2.467 | 0.928 | 0.509 |
| GSGQLGLSVTNQQT | 628 | 1.364 | 6.906 | 1.395 | 0.376 |
| GSGSNMRLSQNQQT | 629 | 1.363 | 0.588 | 0.962 | 0.730 |
| GSGQNLHSGLPQQT | 630 | 1.363 | 1.594 | 1.054 | 0.592 |
| GSSHTLALGQNKQT | 631 | 1.362 | 2.160 | 0.838 | 0.643 |
| GSGQNQHSLPAHRT | 632 | 1.361 | 0.700 | 0.911 | 0.742 |
| GSGQNQGTVYPNQT | 633 | 1.358 | 7.648 | 0.835 | 0.815 |
| GSGQNQQPSLRQST | 634 | 1.356 | 2.905 | 1.315 | 0.554 |
| GSGQNARLKDNQQT | 635 | 1.354 | 2.395 | 0.580 | 0.938 |
| GHAGSTGSGQNQQT | 636 | 1.352 | 2.829 | 1.332 | 1.233 |
| GSGQALSSSGNQQT | 637 | 1.351 | 6.860 | 0.894 | 0.931 |
| GSGASESHRQNQQT | 638 | 1.350 | 0.850 | 0.325 | 0.313 |
| GVGVITSSGQNQQT | 639 | 1.348 | 0.918 | 1.296 | 0.777 |
| GSLYGQSLGQNQQT | 640 | 1.348 | 11.248 | 0.894 | 0.843 |
| GSGQMSDVHGNQQT | 641 | 1.346 | 7.172 | 0.408 | 0.548 |
| GSGQNQQHSSKATT | 642 | 1.345 | 12.248 | 1.350 | 1.401 |
| GSGQNQQTSVSQQT | 643 | 1.342 | 1.614 | 1.030 | 0.913 |
| GSGQKMWKLDNQQT | 644 | 1.341 | 0.000 | 0.990 | 1.418 |
| GSGQNVSMQVNQQT | 645 | 1.341 | 0.000 | 0.357 | 0.251 |
| GSGQNQRATLSNQT | 646 | 1.339 | 1.084 | 0.947 | 0.723 |
| GSGQASSKSANQQT | 647 | 1.339 | 1.138 | 0.500 | 0.175 |
| GSGKNQTPIPKGQT | 648 | 1.339 | 5.077 | 1.306 | 1.154 |
| GSGQNQQTRQEGST | 649 | 1.339 | 0.000 | 0.645 | 0.718 |
| GASSLATSGQNQQT | 650 | 1.337 | 0.703 | 0.423 | 0.217 |
| GSGQRGSLTENQQT | 651 | 1.337 | 2.482 | 0.300 | 0.567 |
| GSEQTRQRGQNQQT | 652 | 1.333 | 2.172 | 0.574 | 0.815 |
| GSGQNQQTLTASKE | 653 | 1.333 | 1.152 | 0.981 | 1.172 |
| GSGTSGKTGKNQQT | 654 | 1.333 | 4.033 | 0.358 | 0.676 |
| GQLVTFTSGQNQQT | 655 | 1.331 | 11.282 | 0.819 | 0.294 |
| GSGQNQQSANKILT | 656 | 1.331 | 3.789 | 0.894 | 1.236 |
| GSGQNQQHHSSHTT | 657 | 1.328 | 2.158 | 0.957 | 0.452 |
| GSGQNQKGMQPNQT | 658 | 1.326 | 3.139 | 0.775 | 1.059 |
| GSGQLVSGLYNQQT | 659 | 1.325 | 0.000 | 0.842 | 0.733 |
| GSSVGVPSGQNQQT | 660 | 1.322 | 4.867 | 0.336 | 1.157 |
| GSGQNQQWDSRRQT | 661 | 1.321 | 0.531 | 1.059 | 0.825 |
| GSEQTRQSGQNQQT | 662 | 1.321 | 0.514 | 0.734 | 0.900 |
| GSGIGSHIPQNQQT | 663 | 1.319 | 0.173 | 0.822 | 0.597 |
| GSGQNQRLHGVDQT | 664 | 1.318 | 4.655 | 0.459 | 0.341 |
| GEVSRVLSGQNQQT | 665 | 1.318 | 0.437 | 1.150 | 0.440 |
| GSGQNQQKVSPLLT | 666 | 1.314 | 1.602 | 0.755 | 0.806 |
| GSGLALERSQNQQT | 667 | 1.311 | 0.486 | 0.618 | 0.096 |
| GPDRIGSSGQNQQT | 668 | 1.308 | 0.426 | 0.654 | 0.342 |
| GSGQNQDHQNKQQT | 669 | 1.308 | 1.470 | 0.510 | 0.761 |
| GSGQNQQTALYNNT | 670 | 1.307 | 0.862 | 0.660 | 0.726 |
| GSGAVHLTAQNQQT | 671 | 1.306 | 1.668 | 0.541 | 0.466 |
| GSLVSTQSGQNQQT | 672 | 1.305 | 1.293 | 1.282 | 0.650 |
| GSGVSARMVQNQQT | 673 | 1.299 | 0.624 | 0.870 | 0.697 |
| GSGQTRMPLANQQT | 674 | 1.296 | 0.790 | 0.447 | 0.273 |
| GSGISSRNMQNQQT | 675 | 1.291 | 6.328 | 1.671 | 0.560 |
| GSGEKVHSGQNQQT | 676 | 1.289 | 0.062 | 0.862 | 0.671 |
| GSGQNQQKLSSMST | 677 | 1.286 | 1.586 | 1.160 | 1.052 |
| GSGQNQQTGQHMRV | 678 | 1.286 | 4.161 | 1.839 | 1.635 |
| GSGMIHITAQNQQT | 679 | 1.285 | 0.105 | 0.678 | 0.276 |
| GSGQNWPALKGQQT | 680 | 1.284 | 2.031 | 1.101 | 1.222 |
| GASHMSISGQNQQT | 681 | 1.284 | 0.462 | 0.404 | 0.374 |
| GSDQNQQLGYSKQT | 682 | 1.283 | 0.000 | 0.853 | 0.660 |
| GIPSIRESGQNQQT | 683 | 1.282 | 0.166 | 0.484 | 0.254 |
| GSGIPSVKFQNQQT | 684 | 1.281 | 0.061 | 0.364 | 0.561 |
| GSGQNQQTSVSQNV | 685 | 1.281 | 0.750 | 0.788 | 0.715 |
| GSGQNQQIGESRMT | 686 | 1.279 | 0.103 | 0.890 | 0.453 |
| GSGSSSMSFQNQQT | 687 | 1.279 | 0.540 | 0.466 | 0.095 |
| GSGQKQERAVSKQT | 688 | 1.277 | 0.000 | 1.174 | 0.732 |
| GCTTRLNSGQNQQT | 689 | 1.276 | 0.000 | 0.184 | 0.618 |
| GSGQNQQIISTKIT | 690 | 1.275 | 0.000 | 0.951 | 0.710 |
| GSGQNQQKSLNGNT | 691 | 1.275 | 8.573 | 0.586 | 0.851 |
| GSGIPAPRLQNQQT | 692 | 1.273 | 4.162 | 0.583 | 0.396 |
| GSGQIRESMGNQQT | 693 | 1.270 | 1.676 | 0.833 | 0.523 |
| GSGQNSGVHENQQT | 694 | 1.268 | 0.587 | 0.871 | 0.377 |
| GSGQNIGHSLPQQT | 695 | 1.264 | 6.183 | 0.740 | 0.478 |
| GSGERSISVQNQQT | 696 | 1.264 | 1.619 | 0.598 | 0.173 |
| GSGLKPNVLQNQQT | 697 | 1.263 | 0.975 | 0.701 | 0.268 |
| GSGQVAYAQGNQQT | 698 | 1.259 | 1.309 | 0.734 | 0.313 |
| GSGQSSYGSGNQQT | 699 | 1.257 | 1.686 | 1.161 | 0.456 |
| GSGQNQAMTHGDQT | 700 | 1.257 | 1.878 | 0.357 | 0.259 |
| GSGQNQALVSMGQT | 701 | 1.255 | 1.876 | 0.987 | 0.560 |
| GSGQNPSEMRGQQT | 702 | 1.252 | 1.454 | 1.293 | 1.094 |
| GSGQNQQSHLRINT | 703 | 1.251 | 4.583 | 1.022 | 0.718 |
| GYTRLETSGQNQQT | 704 | 1.250 | 1.323 | 0.841 | 0.297 |
| GSGQSYDMRGNQQT | 705 | 1.248 | 0.567 | 0.588 | 0.368 |
| GSRTTQDIGQNQQT | 706 | 1.247 | 0.000 | 0.685 | 0.280 |
| GSGHPYKAAQNQQT | 707 | 1.246 | 0.000 | 0.872 | 0.507 |
| GRLSNAHGGQNQQT | 708 | 1.245 | 0.839 | 1.036 | 0.725 |
| GSGQNQRAVLNDQT | 709 | 1.242 | 3.023 | 0.556 | 0.259 |
| GGSHTYGGGQNQQT | 710 | 1.241 | 13.065 | 0.982 | 0.730 |
| GSSVNSMIGQNQQT | 711 | 1.239 | 0.000 | 0.976 | 0.580 |
| GNSSMMGSGQNQQT | 712 | 1.239 | 3.856 | 0.656 | 0.364 |
| GNRDRPSSGQNQQT | 713 | 1.239 | 3.947 | 0.298 | 0.178 |
| GSGNMHASRQNQQT | 714 | 1.238 | 3.878 | 0.782 | 0.687 |
| GFIFPKVSGQNQQT | 715 | 1.237 | 0.000 | 1.764 | 0.692 |
| GSGQNQQLKNSTST | 716 | 1.235 | 1.703 | 1.063 | 0.538 |
| GSGQNQQSQYMPRT | 717 | 1.234 | 0.401 | 0.549 | 0.520 |
| GSGQRMADIGNQQT | 718 | 1.233 | 2.539 | 0.352 | 0.427 |
| GSGQNQSHYPSQQT | 719 | 1.228 | 4.315 | 0.644 | 0.402 |
| GSDGKMHRGQNQQT | 720 | 1.227 | 0.000 | 1.826 | 0.776 |
| GSGSVGFIGQNQQT | 721 | 1.227 | 8.261 | 0.689 | 0.445 |
| GLHGMTLSGQNQQT | 722 | 1.226 | 3.552 | 0.470 | 0.338 |
| GSDQSKRGDSNQQT | 723 | 1.225 | 0.639 | 0.479 | 0.267 |
| GSLFLATGGQNQQT | 724 | 1.220 | 0.000 | 0.775 | 0.485 |
| GSGQNQQPSAFSKT | 725 | 1.220 | 4.906 | 1.309 | 0.754 |
| GSGQLPQSGLNQQT | 726 | 1.218 | 1.504 | 0.641 | 0.318 |
| GSGSKQNALQNQQT | 727 | 1.216 | 2.010 | 0.941 | 0.594 |
| GSGQRRELSQNQQT | 728 | 1.215 | 1.791 | 0.622 | 0.396 |
| GSGQREPKASNQQT | 729 | 1.214 | 2.793 | 0.399 | 0.520 |
| GSGQNQQHPSTQQT | 730 | 1.205 | 1.552 | 1.017 | 0.680 |
| GSQSTLGLGQNQQT | 731 | 1.204 | 3.246 | 0.594 | 0.400 |
| GSGQNQQMPGLSST | 732 | 1.204 | 1.887 | 0.234 | 0.181 |
| GSGQNQQTVGGKNL | 733 | 1.203 | 0.128 | 0.777 | 1.051 |
| GSSREFHSGQNQQT | 734 | 1.203 | 1.591 | 0.688 | 0.474 |
| GSGQNQQTVPSNLV | 735 | 1.201 | 0.791 | 0.434 | 0.281 |
| GSGQNAYSSQAQQT | 736 | 1.201 | 12.096 | 0.629 | 0.216 |
| GSGQNKDHSTRRQT | 737 | 1.197 | 0.000 | 0.384 | 0.477 |
| GQLGSVGSGQDQQT | 738 | 1.196 | 0.000 | 1.020 | 0.437 |
| GSGQHAAPGHNQQT | 739 | 1.195 | 5.999 | 0.600 | 0.199 |
| GSGQNQQTSQSPPT | 740 | 1.194 | 1.208 | 0.851 | 0.478 |
| GSGNYRDHEQNQQT | 741 | 1.193 | 7.389 | 0.287 | 0.222 |
| GSGQHSNQHVNQQT | 742 | 1.192 | 1.453 | 0.955 | 0.558 |
| GSGQTARNGINQQT | 743 | 1.192 | 2.030 | 1.002 | 0.472 |
| GSGQNQQHYGSQGT | 744 | 1.189 | 0.453 | 1.345 | 0.379 |
| GSGSPQASRQNQQT | 745 | 1.189 | 6.782 | 0.923 | 0.542 |
| GSGESHSMGKNQQT | 746 | 1.188 | 9.809 | 1.381 | 0.611 |
| GSGQSHSLETNQQT | 747 | 1.188 | 1.319 | 0.520 | 0.363 |
| GTEQTRQSGQNQQT | 748 | 1.188 | 0.132 | 0.756 | 0.756 |
| GSGRHLASVQNQQT | 749 | 1.187 | 1.024 | 0.654 | 0.606 |
| GLGSKNHSGQNQQT | 750 | 1.187 | 5.046 | 0.825 | 0.224 |
| GSGQNQQTSHFPSA | 751 | 1.185 | 0.325 | 0.969 | 0.907 |
| GSGQLSGIPQNQQT | 752 | 1.185 | 1.382 | 1.025 | 0.643 |
| GSGQNQQAPHKKET | 753 | 1.180 | 0.598 | 0.994 | 0.689 |
| GSGQNQQTLRGSLE | 754 | 1.179 | 1.812 | 0.853 | 0.354 |
| GSTAMTSHGQNQQT | 755 | 1.178 | 1.435 | 0.551 | 0.438 |
| GSPGVSPSGQNQQT | 756 | 1.178 | 3.006 | 0.853 | 1.160 |
| GSGQNQQTGSSSRV | 757 | 1.176 | 0.580 | 0.995 | 1.128 |
| GSGQHLPLLGNQQT | 758 | 1.175 | 1.739 | 0.519 | 0.347 |
| GSDHSHRGGQNQQT | 759 | 1.174 | 0.504 | 0.818 | 0.331 |
| GSGIVTKLGQNQQT | 760 | 1.174 | 10.571 | 0.599 | 0.242 |
| GSGQDVTKTGNQQT | 761 | 1.173 | 4.523 | 0.531 | 0.035 |
| GSGQNQQSHGRIGT | 762 | 1.173 | 5.117 | 0.607 | 0.455 |
| GSGQNQQINHRSPT | 763 | 1.173 | 0.748 | 0.259 | 0.220 |
| GSGDDSRVGQNQQT | 764 | 1.172 | 0.191 | 0.466 | 0.156 |
| GSGQSTLKRINQQT | 765 | 1.168 | 13.442 | 0.534 | 1.184 |
| GSGSQHSKAQNQQT | 766 | 1.168 | 0.312 | 0.638 | 0.916 |
| GSGQNQQHASSNNT | 767 | 1.166 | 7.155 | 0.789 | 0.896 |
| GSRTYQVSGQNQQT | 768 | 1.164 | 1.853 | 0.638 | 0.641 |
| GSGQNQGLLSSPQT | 769 | 1.164 | 0.000 | 0.707 | 0.417 |
| GSGGGLQHNQNQQT | 770 | 1.163 | 4.098 | 1.137 | 0.778 |
| GSGQNQQTTAATRM | 771 | 1.163 | 3.925 | 0.947 | 1.005 |
| GSGQNQRASILVQT | 772 | 1.162 | 3.632 | 0.531 | 0.569 |
| GSGQNLGLLGAQQT | 773 | 1.161 | 1.458 | 0.524 | 0.226 |
| GSLDLGRSGQNQQT | 774 | 1.160 | 3.283 | 1.002 | 0.505 |
| GNSQVKVSGQNQQT | 775 | 1.158 | 4.930 | 1.422 | 0.728 |
| GSSGSHQYGQNQQT | 776 | 1.155 | 0.000 | 1.129 | 0.794 |
| GSGQNQQQRDGTLT | 777 | 1.152 | 0.387 | 0.760 | 0.730 |
| GRGQHVSVANNQQT | 778 | 1.152 | 1.896 | 1.032 | 0.589 |
| GDSSSRISGQNQQT | 779 | 1.151 | 3.787 | 0.916 | 0.348 |
| GSGQNQQHSLSSQT | 780 | 1.150 | 3.844 | 0.700 | 0.730 |
| GSLMDVHRGQNQQT | 781 | 1.150 | 0.387 | 1.009 | 0.238 |
| GSIQYQSSGQNQQT | 782 | 1.147 | 2.601 | 1.074 | 1.191 |
| GLGSKNPSGQNQQT | 783 | 1.147 | 1.629 | 1.184 | 0.424 |
| GSGQLVLTLQNQQT | 784 | 1.143 | 0.000 | 0.336 | 0.336 |
| GSGQNQQTSQPLPG | 785 | 1.141 | 0.080 | 0.748 | 0.530 |
| GSGQNQQNLGKLNT | 786 | 1.141 | 0.000 | 0.919 | 0.687 |
| GTTAHQPSGQNQQT | 787 | 1.138 | 0.211 | 0.726 | 0.275 |
| GSGQNRAQIGTQQT | 788 | 1.138 | 0.469 | 0.776 | 0.654 |
| GSGQYVHVSSNQQT | 789 | 1.137 | 1.803 | 0.739 | 0.366 |
| GSGQNQQTAHAFNI | 790 | 1.132 | 3.404 | 0.699 | 0.729 |
| GSGQNQRTMVATQT | 791 | 1.130 | 1.122 | 0.649 | 0.554 |
| GSGQNPIRGAMQQT | 792 | 1.126 | 1.327 | 1.296 | 0.427 |
| GSGYVITGSQNQQT | 793 | 1.125 | 6.271 | 0.971 | 0.248 |
| GRGPKQSNIQNQQT | 794 | 1.125 | 0.737 | 0.771 | 2.490 |
| GSGQNQQTMLGKPC | 795 | 1.125 | 0.047 | 1.090 | 0.992 |
| GSGQNQQVGSTVRT | 796 | 1.124 | 2.040 | 0.918 | 0.614 |
| GNVTTQKSGQNQQT | 797 | 1.122 | 2.546 | 1.215 | 0.922 |
| GSGNPVSHLQNQQT | 798 | 1.121 | 1.037 | 0.583 | 0.310 |
| GSLSHMESGQNQQT | 799 | 1.120 | 0.829 | 0.489 | 0.265 |
| GRAPTNLSGQNQQT | 800 | 1.118 | 0.687 | 0.757 | 0.169 |
| GSGQNQQTVMTARA | 801 | 1.117 | 1.535 | 0.995 | 0.843 |
| GSGMPASRLQNQQT | 802 | 1.117 | 1.689 | 0.790 | 0.372 |
| GVVRNHQSGQNQQT | 803 | 1.116 | 5.801 | 0.899 | 0.868 |
| GSGQNQHSVQVRQT | 804 | 1.116 | 1.909 | 0.782 | 0.916 |
| GSGQNTGHLTMQQT | 805 | 1.114 | 0.078 | 1.026 | 0.595 |
| GSGQNQQYAGKILT | 806 | 1.112 | 0.300 | 1.078 | 0.431 |
| GSGNPHVRNQNQQT | 807 | 1.112 | 0.873 | 0.732 | 0.755 |
| GSGQNGGSSNRQQT | 808 | 1.109 | 2.594 | 1.255 | 0.844 |
| GSGQRLSQGVNHQT | 809 | 1.108 | 3.394 | 0.931 | 1.141 |
| GSGQNAHAKEGQQT | 810 | 1.108 | 0.000 | 0.875 | 1.179 |
| GSSPAPNSGQNQQT | 811 | 1.106 | 2.229 | 0.719 | 0.368 |
| GLAHKTSSGQNQQT | 812 | 1.106 | 0.915 | 0.427 | 0.690 |
| GSGQNQQTPGAHKT | 813 | 1.105 | 3.827 | 0.957 | 0.277 |
| GSGQNQQSLSGSET | 814 | 1.105 | 0.735 | 0.745 | 0.883 |
| GSGQNQQSTGTSRT | 815 | 1.103 | 4.054 | 1.209 | 0.935 |
| GSGQNQQTVQSNLV | 816 | 1.103 | 2.350 | 0.577 | 0.698 |
| GSGQNQQLGSRQCT | 817 | 1.102 | 0.183 | 0.987 | 0.407 |
| GSGQNQYLRLELQT | 818 | 1.101 | 0.000 | 0.416 | 0.839 |
| GSGQNQQTSPRLQT | 819 | 1.100 | 0.795 | 1.156 | 1.091 |
| GSGQNQQTTSSNMT | 820 | 1.099 | 0.569 | 0.638 | 0.698 |
| GTASTYNSGQNQQT | 821 | 1.099 | 2.560 | 0.250 | 0.625 |
| GSGQNQQTMPQHKI | 822 | 1.097 | 2.394 | 0.479 | 0.197 |
| GSGQSHLHTGNQQT | 823 | 1.096 | 2.584 | 0.721 | 0.295 |
| GVKGVGHSGQNQQT | 824 | 1.096 | 2.485 | 0.994 | 0.783 |
| GSGKVTKQSQNQQT | 825 | 1.095 | 0.000 | 0.928 | 1.035 |
| GSGQNQQTALEKSI | 826 | 1.092 | 0.000 | 0.625 | 0.702 |
| GSGYKDTYGQNQQT | 827 | 1.091 | 0.854 | 0.717 | 0.448 |
| GSGQNQQSGTELST | 828 | 1.090 | 5.673 | 1.021 | 0.742 |
| GSGQNTGQHMMQQT | 829 | 1.090 | 1.058 | 1.147 | 0.917 |
| GSGKNQQRPGLDQT | 830 | 1.089 | 1.557 | 0.583 | 0.385 |
| GSGQSREISLNQQT | 831 | 1.088 | 6.954 | 0.594 | 0.282 |
| GTPTSPSSGQNQQT | 832 | 1.086 | 4.558 | 0.833 | 0.662 |
| GKPAGGLSGQNQQT | 833 | 1.085 | 2.805 | 0.708 | 0.739 |
| GSGQNHRSADMQQT | 834 | 1.084 | 12.001 | 0.417 | 0.212 |
| GSGQNQQTLPSLSL | 835 | 1.084 | 1.758 | 0.527 | 0.175 |
| GSPYMGATGQNQQT | 836 | 1.083 | 5.364 | 0.918 | 0.254 |
| GSGHAKAVGQNQQT | 837 | 1.081 | 4.357 | 0.703 | 0.824 |
| GHMKGVTSGQNQQT | 838 | 1.081 | 2.814 | 0.807 | 0.413 |
| GSGQNQKILTLDQT | 839 | 1.080 | 0.371 | 0.291 | 0.314 |
| GSGQNQQTKVGHSA | 840 | 1.079 | 1.256 | 0.669 | 1.019 |
| GIARTTISGQNQQT | 841 | 1.078 | 1.783 | 0.819 | 0.330 |
| GSGQNQQTSVGERT | 842 | 1.077 | 3.737 | 0.648 | 0.534 |
| GSGQNQQTMIANIR | 843 | 1.076 | 0.000 | 0.379 | 0.458 |
| GDMTRSSSGQNQQT | 844 | 1.075 | 0.802 | 1.145 | 1.038 |
| GSGHMSDLRQNQQT | 845 | 1.073 | 4.291 | 0.555 | 0.328 |
| GRGAVMASGQNQQT | 846 | 1.072 | 0.923 | 0.783 | 0.605 |
| GSGQNQQLSGKSVT | 847 | 1.070 | 1.524 | 1.276 | 0.930 |
| GSHTLVVSGQNQQT | 848 | 1.069 | 1.535 | 0.671 | 0.748 |
| GSGPWSAGLQNQQT | 849 | 1.067 | 0.947 | 0.700 | 0.539 |
| GSGQHSPHALNQQT | 850 | 1.064 | 1.412 | 0.885 | 0.573 |
| GSGQNQQPNSGSMT | 851 | 1.064 | 0.925 | 0.588 | 0.339 |
| GSGLAHLGGQNQQT | 852 | 1.064 | 2.191 | 0.749 | 0.794 |
| GSSVRYEPKQNQQT | 853 | 1.063 | 1.564 | 0.450 | 0.501 |
| GSGQNQQARPLELT | 854 | 1.061 | 0.059 | 0.389 | 0.252 |
| GSGQPRSTGINQQT | 855 | 1.061 | 0.693 | 0.650 | 0.542 |
| GSGQNQANWVKVQT | 856 | 1.059 | 0.126 | 0.683 | 0.532 |
| GSGHLFQSGQNQQT | 857 | 1.057 | 0.615 | 0.751 | 0.386 |
| GSGQNRGISISQQT | 858 | 1.057 | 2.166 | 0.686 | 0.566 |
| GSGTHYDNRQNQQT | 859 | 1.054 | 0.072 | 0.612 | 0.486 |
| GSGQNQQTSTTPLP | 860 | 1.052 | 2.823 | 0.828 | 0.741 |
| GSGQVHASQVNQKT | 861 | 1.049 | 0.503 | 0.855 | 0.767 |
| GSSGHRESGQNQQT | 862 | 1.048 | 4.398 | 0.641 | 0.691 |
| GLSAEKSSGQNQQT | 863 | 1.047 | 7.203 | 0.629 | 0.303 |
| GSGQEHRSLANQQT | 864 | 1.046 | 0.000 | 0.507 | 0.344 |
| GSGQTVVRIANQQT | 865 | 1.046 | 4.156 | 0.661 | 0.390 |
| GSGQNVSSVHRQQT | 866 | 1.045 | 0.712 | 0.383 | 0.271 |
| GSGASRMSIQNQQT | 867 | 1.045 | 0.111 | 0.801 | 0.417 |
| GVAFIGSSGQNQQT | 868 | 1.043 | 0.000 | 0.744 | 0.648 |
| GSGQNQQTVPTRQT | 869 | 1.040 | 1.207 | 0.629 | 0.138 |
| GSGQAAKSSQNQQT | 870 | 1.036 | 0.681 | 0.778 | 0.737 |
| GSGQNQQVAIRTST | 871 | 1.035 | 2.447 | 0.963 | 0.370 |
| GSVHMQNAGQNQQT | 872 | 1.034 | 3.608 | 1.004 | 0.625 |
| GSGMRQAGVQNQQT | 873 | 1.032 | 0.811 | 0.736 | 0.775 |
| GSGQNQQVGGKTVT | 874 | 1.032 | 6.195 | 1.094 | 0.821 |
| GVHDMRVSGQNQQT | 875 | 1.032 | 8.083 | 1.171 | 0.818 |
| GSGQHVSVANNQQT | 876 | 1.029 | 5.734 | 0.974 | 0.577 |
| GSAAMSVRGQNQQT | 877 | 1.029 | 2.386 | 0.202 | 0.287 |
| GVSRGGPSGQNQQT | 878 | 1.028 | 1.611 | 0.750 | 0.591 |
| GSGQMVHTIGNQQT | 879 | 1.026 | 1.328 | 0.406 | 0.430 |
| GRGGSMAETQNQQT | 880 | 1.024 | 2.853 | 0.799 | 0.669 |
| GSGHTNPTRQNQQT | 881 | 1.021 | 0.688 | 0.726 | 0.807 |
| GSGEAARYEQNQQT | 882 | 1.020 | 0.000 | 0.107 | 0.125 |
| GSGQNERHLVLQQT | 883 | 1.019 | 5.354 | 0.416 | 0.150 |
| GSGQNQQSKQQVLT | 884 | 1.019 | 1.494 | 1.428 | 1.256 |
| GSGQARAHRGNQQT | 885 | 1.017 | 0.000 | 0.254 | 0.386 |
| GSGQNQQPLDTSRT | 886 | 1.015 | 0.775 | 0.491 | 0.376 |
| GSGQNQQLANMVTT | 887 | 1.014 | 1.739 | 1.253 | 0.987 |
| GSGQMKDLHRNQQT | 888 | 1.014 | 1.068 | 0.587 | 0.506 |
| GSGQNQHLSSFVQT | 889 | 1.013 | 0.110 | 1.090 | 0.364 |
| GSGQNQQPSSRVTT | 890 | 1.012 | 2.179 | 0.784 | 0.504 |
| GSGQNQQLAITLGT | 891 | 1.011 | 0.000 | 0.877 | 0.143 |
| GSGQNQQTVGNPAT | 892 | 1.008 | 3.014 | 0.856 | 0.395 |
| GSGQNQGRAHPMQT | 893 | 1.007 | 2.364 | 0.684 | 0.453 |
| GSGQLIASVVNQQT | 894 | 1.005 | 0.086 | 0.197 | 0.359 |
| GSSVRSLVGQNQQT | 895 | 1.004 | 3.840 | 0.412 | 0.608 |
| GGAGSAHSGQNQQT | 896 | 1.003 | 6.108 | 0.474 | 1.092 |
| GSDQNQQTMSSTRT | 897 | 1.003 | 2.428 | 1.306 | 0.835 |
| GSGQNQQMAGAERT | 898 | 1.003 | 1.784 | 1.307 | 0.762 |
| GSLGNLQRGQNQQT | 899 | 1.003 | 0.895 | 0.947 | 0.385 |
| GSGPSISHGQNQQT | 900 | 1.000 | 0.000 | 0.614 | 0.665 |
| GSGQNQQT | 6406 | 1.000 | 1.000 | 1.000 | 1.000 |
| GSGQNQQSSENVQT | 901 | 0.998 | 0.000 | 1.307 | 0.675 |
| GSGQNQQTGQATHN | 902 | 0.996 | 2.199 | 0.877 | 0.527 |

A second cross-species evolution screen was performed using an AAV capsid variant library with a modification in loop 1V introduced as described above and passaging it once through NHPs (passage 1) and then subsequently injected it into two different strains of mice (passage 2), C57BV6 and BALB/c. The fold-enrichment foreach variant in the brain of each mouse species was calculated by systematic NGS enrichment analysis following RNA recovery and RT-PCR amplification. The fold enrichment values in the second passage in mice were compared to those fold enrichment values from the second pass that was performed in NHPs as described above. As shown in Table 17, when comparing the second pass fold enrichment values in the mice versus NHPs, 12 variants were identified that had a fold-enrichment value greater than 10 in all three animal groups. Further, 10 of these 12 variants comprised the SPH motif and a positive residue in one of the next three subsequent residues (Table 17).

TABLE 17
NGS fold-enrichment of AAV capsid variants from a second passage (P2) in NHPs or
mice (C57BI/6 or BALB/c) following a first passage in NHPs

| | SEQ | Fold enrichment | Fold enrichment | Fold enrichment |
|---|---|---|---|---|
| | ID | over AAV9 | over AAV9 | over AAV9 in |
| Peptide Sequence | NO: | in NHP P2 | in BALB/c P2 | C57BI/6 P2 |
| VSGSPHSKAQNQQT | 903 | 99.76 | 92.99 | 34.29 |
| CSGSPHSKAQNQQT | 904 | 85.1 | 66.74 | 22.19 |
| GSGSPHSKAQNQQT | 200 | 56.33 | 44.58 | 14.48 |
| GSGSPHRKAQNQQT | 905 | 46.39 | 42.47 | 14.11 |
| GRGSPHSKAQNQQT | 906 | 43.68 | 59.65 | 28.13 |
| GHDSPHKSGQNQQT | 201 | 33.96 | 59.14 | 27.15 |
| GSGSPHSKAKNQQT | 208 | 31.27 | 41.51 | 14 |
| GSGSPHSKAQNKQT | 907 | 29.52 | 44.1 | 13.69 |
| GSGSPHSKAQTQQT | 908 | 24.27 | 41.75 | 18 |
| GQDSPHKSGQNQQT | 204 | 22.7 | 32.37 | 16.02 |
| GSGSTHASRQNQQT | 909 | 11.04 | 23.71 | 10.67 |
| GHDSQHKSGQNQQT | 404 | 10.36 | 21.3 | 13.55 |

Following the second passage in mice, a synthetic library was generated using those variants that demonstrated a fold-change: in enrichment relative to wild-type AAV9 that was above 10 in the brain of either strain of mice, as measured by systematic NGS enrichment analysis following RNA recovery and RT-PCR amplification. There were approximately 500 variants in this synthetic library. This synthetic library was the injected back into both strains of mice (C57B6 and BALB/c; passage 3). RNA was recovered from the mouse brains, RT-PCR amplification was performed, and fold-enrichment relative to wild-type AAV9 was calculated by NGS analysis, which is provided in Table 18. As shown in Table 18, the variants with the greatest fold-enrichment in the brain in each strain, were highly correlated across strains (R²=0.8458).

TABLE 18
NGS fold-enrichment of AAV capsid variants in the brain from a third passage
(P3) in mice (C57BI/6 or BALB/c) following a first and second passage in mice

| | | Fold enrichment | Fold enrichment | |
|---|---|---|---|---|
| | SEQ | over AAV9 in | over AAV9 in | |
| Peptide Sequence | ID NO: | BALB/c | C57BI/6 | Average |
| GSGSPHKYGQNQQT | 910 | 150.445 | 103.488 | 126.966 |
| GSGSPHKFGQNQQT | 911 | 73.364 | 60.304 | 66.834 |
| GHDSPHKSGQNQQT | 201 | 82.460 | 51.125 | 66.792 |
| GSGSPHSKAQNQQT | 200 | 60.312 | 65.853 | 63.083 |
| VSGSPHKFGQNQQT | 912 | 60.186 | 59.142 | 59.664 |
| GSGSPHSKAQNHQT | 913 | 63.486 | 51.647 | 57.566 |
| VSGSPHSKAQNQQT | 903 | 73.555 | 37.429 | 55.492 |
| GQDSPHKSGQNQQT | 204 | 63.898 | 43.752 | 53.825 |
| GSGSPHSKAQHQQT | 914 | 45.309 | 45.600 | 45.454 |
| GSGSPHKTYQNQQT | 915 | 50.283 | 35.460 | 42.871 |
| GSGSPHSKAQTQQT | 908 | 43.120 | 39.098 | 41.109 |
| VSGSPHASRQNQQT | 916 | 46.572 | 32.480 | 39.526 |
| GSGSPHSKAQNKQT | 907 | 39.848 | 35.596 | 37.722 |
| GSGSPHKEGKNQQT | 917 | 31.948 | 34.899 | 33.423 |
| GSGSPHASRQNQHT | 918 | 28.145 | 30.928 | 29.537 |
| GSHSPHKSGQNQQT | 919 | 22.948 | 35.412 | 29.180 |
| GSGQNQQRRMSPST | 920 | 4.576 | 53.520 | 29.048 |
| GSGSPHASRQNQQT | 205 | 28.866 | 29.139 | 29.003 |
| GSGSPHSKPQNQQT | 921 | 26.958 | 28.599 | 27.779 |
| GSGSPHKEGQKQQT | 922 | 39.597 | 14.927 | 27.262 |
| VSGSPHGARQNQQT | 923 | 30.985 | 22.634 | 26.810 |
| GSGSPHSKAQKQQT | 924 | 25.052 | 27.459 | 26.256 |
| GHSSPHRSGQNQQT | 212 | 16.982 | 35.081 | 26.032 |
| GSGSPHSKAKNQQT | 208 | 21.069 | 25.711 | 23.390 |
| GSHSPHKRGQNQQT | 925 | 24.054 | 20.262 | 22.158 |
| GRGSPHSKAQNQQT | 906 | 20.939 | 22.720 | 21.830 |
| GQSSPHRSGQNQQT | 229 | 9.916 | 26.608 | 18.262 |
| GSGQNRQRLKGLET | 926 | 3.937 | 31.022 | 17.480 |
| GSGSPHKLGQNQQT | 927 | 18.905 | 14.732 | 16.818 |
| GSGSPHKTSKNQQT | 928 | 14.654 | 17.606 | 16.130 |
| GSGSPHKIGQNQQT | 929 | 16.999 | 14.794 | 15.897 |
| GSGSPHKKNQNQQT | 209 | 25.633 | 5.605 | 15.619 |
| GSGSPHASRQNKQT | 206 | 10.738 | 20.347 | 15.542 |
| GSGSPHTRGQNQQT | 214 | 16.899 | 13.869 | 15.384 |
| GSGQDSPHVRNQQT | 930 | 15.340 | 14.646 | 14.993 |
| GSGSPHKTSQNQQT | 931 | 20.428 | 8.818 | 14.623 |
| GSGSPHASRKNQQT | 932 | 13.799 | 12.749 | 13.274 |
| GSGSPHASRQKQQT | 211 | 13.624 | 11.188 | 12.406 |
| GSHSPHKSGQKQQT | 933 | 6.700 | 17.736 | 12.218 |
| GSGSPHKTSQKQQT | 934 | 12.621 | 11.720 | 12.170 |
| GSGSPHVRGQNKQT | 935 | 13.174 | 11.017 | 12.095 |
| GSGSPHKTTQNQQT | 936 | 9.722 | 13.381 | 11.552 |
| CSGSPHSKAQNQQT | 904 | 11.772 | 9.447 | 10.610 |
| GSGPVRALRQNQQT | 937 | 3.369 | 17.431 | 10.400 |
| GSGSPHVRGQKQQT | 938 | 7.573 | 12.498 | 10.036 |
| GSGSPHRKAQNQQT | 905 | 12.308 | 7.349 | 9.828 |
| GRGSPHASRQNQQT | 318 | 11.903 | 6.780 | 9.342 |
| CSGSPHKTSQNQQT | 939 | 11.167 | 6.631 | 8.899 |
| CSHSPHKSGQNQQT | 940 | 11.356 | 6.304 | 8.830 |
| GSGSPHSKDQNQQT | 604 | 3.492 | 10.236 | 6.864 |

Taken together, these results demonstrate that after 3 rounds of screening of this AAV9 variant library with loop IV modifications in NHP and mice, many AAV capsid variants outperformed the wild-type AAV9, for example, in penetration of the blood brain barrier (BBB) and spinal cord expression. These capsid variants were able to cross-species, evidenced by expression and tropism in the NHP brain/spinal cord as well as in the brain of two different mouse species.

### Example 2. Individual Capsid Characterization in Mice

The goal of these experiments was to determine the transduction level, tropism, ability to cross the blood brain barrier, and overall spatial distribution in the central nervous system (CNS) of 2 capsid variants selected from the study described in Example I relative to AAV9 following intravenous injection in mice. The 2 capsid variants were TTM-001 (SEQ ID NO: 981 (amino acid) and 983 (DNA), comprising SEQ ID NO: 941) and TTM-002 (SEQI1D NO: 982 (amino acid) and 984 (DNA), comprising SEQ ID NO: 2), as outlined in Table 3 above. The amino acid and DNA sequences of TTM-001 and TTM-002 are provided, e.g., in Tables 4 and 5, respectively.

AAV particles were generated with each of these capsid variants encapsulating a luciferase-EGFP transgene driven by a CMV/chicken beta actin promoter in a single stranded viral genome. Each capsid variant and AAV9 control were tested by intravenously administering by tail vein injection, the AAV particle formulation at 5e11 VG/dose (2.5E13 vg/kg) to three female BALB/c mice. The in-life period was 28 days and then various CNS and peripheral tissues were collected for measuring transgene mRNA, transgene protein, and viral DNA (biodistribution).

At 28 days post-injection of the AAV particles encapsulated in the TTM-M01 capsid variant (AAV_TTM401I), mice were injected with luciferin and their brains were harvested for IVIS imaging. Robust luciferase signal was observed in mice injected with AAV particles encapsulated in the TTM-001 capsid variant, and this was greatly increased relative to AAV particles encapsulated in the wild-type AAV9 control capsid.

The brains isolated from mice injected with the AAV particles encapsulated in the TTM-001 capsid variant (AAV_TTM-001) or the TTM-002 capsid variant (AAV_TTM402) were assayed by qPCR for the presence of transgene RNA as a measure of transgene expression, and the presence of viral DNA as a measure of viral genome levels. Data were provided as fold over AAV9 (Table 19). As shown in Table 19, when compared to the wild-type AAV9 capsid control. TTM-001 and TM-002 demonstrated a 30-fold and 66-fold increase, respectively, in transgene mRNA levels and expression in the brain, indicative of enhanced payload delivery. This correlated with a 32-fold (TTM-001) and 47-fold (TTM-002) increase, respectively, in viral genome (DNA) concentrations in the brain relative to the AAV9 capsid control, which is indicative of enhanced CNS tropism and transduction (Table 19).

TABLE 19
Transgene mRNA and viral genome levels (DNA)
in mice relative to the AAV9 control

| Measure | Tissue | AAV9 | TTM-001 | TTM-002 |
|---|---|---|---|---|
| mRNA (transgene | Brain | 1.0 | 30.4503 | 66.2161 |
| expression) | | | | |
| DNA (viral genome | Bram | 1.0 | 32.0315 | 47.2810 |
| quantification) | | | | |
| mRNA (transgene | Liver | 1.0 | 1.2356 | 0.2016 |
| expression) | | | | |
| DNA (viral genome | Liver | 1.0 | 0.4802 | 0.0277 |
| quantification) | | | | |

The brain tissues and spinal cords of the mice were also subjected to anti-GFP immunohistochemistry staining to evaluate overall CNS tropism and biodistribution. Immunohistochemical staining correlated with the qPCR analysis, as TTM-001 and TTM402 showed significantly stronger staining and payload expression in the brain and spinal cord, as compared to the AAV9 control. More specifically. TTM-01 and TTM402 demonstrated localization and strong payload expression and transduction in the mid-brain region, with increased staining observed in the hippocampus and thalamus, as well as in the brain stem, compared to AAV9. Less staining was observed in the cortical regions of the brain compared to the midbrain. However, staining in these cortical regions was stronger for TTM-001 and TTM-002 compared to the AAV9 control. It also appeared that the TTM-001 and TTM4002 capsid variants were able to transduce non-neuronal cells, including glial cells and oligodendrocytes. With respect to the spinal cord, staining and payload expression for TTM-01 and TTM-002 were localized to the ventral horns of the grey matter.

Peripheral tissues were also isolated from the mice intravenously injected with the AAV particles encapsulated in the TTM-001 capsid variant or the TTM-002 capsid variant for analysis by qPCR and/or GFP immunohistochemical staining. Transgene mRNA levels and viral genome DNA levels were quantified in the liver by qPCR and the fold over AAV9 was calculated for each capsid variant (Table 19). TTM-001 resulted in similar levels of payload expression (mRNA levels) as compared to wild-type AAV9, but only half as much viral genome DNA was quantified in the liver compared to AAV9, TTM-002 demonstrated greatly reduced mRNA and viral genome DNA levels in the liver compared to AAV9. GFP immunohistochemical staining of the spleen, heart, skeletal muscle, kidneys, and lungs of mice injected with AAV particles encapsulated in the TTM-001 capsid variant or the TTM-002 capsid variant showed similar levels of payload expression as compared to those mice injected with AAV particles encapsulated in the wild-type AAV9 control capsid.

Taken together, these data demonstrate that TTM-W01 and TTM-002 are enhanced CNS tropic capsids in mice that can infect non-neuronal cells. Additionally, these capsid variants were able to successfully penetrate the blood brain barrier following intravenous injection.

### Example 3. Maturation of TTM-001 and TTM-002 Caosid in Mice

This Example describes maturation of the TTM-001 (SEQ ID NO: 981 (amino acid) and 983 (DNA), comprising SEQ ID NO: 941) and TTM-002 (SEQ ID NO: 982 (amino acid) and 984 (DNA), comprising SEQ ID NO: 2) capsid variants to further enhance their transduction and biodistribution in the central nervous system and evolve the AAV capsid variants to provide further cross-species compatibility. Two approaches were used to mature the TTM401 and TTM-002 capsid sequences in order to randomize and mutate within and around the peptide insert comprised within loop IV of the capsid variant. As many of the AAV capsid variants that demonstrated the greatest fold-enrichment in the NHP brain relative wild-type AAV9 comprised an SPH motif in the same position (e.g., immediately subsequent to position 455, relative to a reference sequence numbered according to the amino acid sequence of SEQ ID NO: 138) (see Example 1), the SPH motif was not mutated in either approach to mature the TTM-001 and TTM-02 capsid variants. In the fust maturation approach, sets of three contiguous amino acids were randomized across the mutagenesis region in the TTM-001 and TTM-002 sequences, which spanned from position 450 to position 466, numbered according to SEQ ID NO: 981 and 982. In the second maturation approach, mutagenic primers were used to introduce point mutations at a low frequency, scattered across the mutagenesis region in the TTM-001 and TTM-002 sequences ranging from position 449 to position 466, numbered according to SEQ ID NO: 981 and 982. AAV capsid variants arising from each maturation approach for TTM-001 were pooled together and AAV capsid variants arising from each maturation approach for TTM-002 were also pooled together, for subsequent testing and characterization in mice.

The library of pooled matured AAV capsid variants generated from TTM-001 or library of pooled matured AAV capsid variants generated from the TTM-002 matured AAV capsid variant each were intravenously injected into the tail vein of three female CD-1 Outbred mice (Charles River) at a dose of 1.0×10¹² VG/dose. After 14-days in life, the brains of the mice were isolated and RNA was extracted. Following RNA recovery and RT-PCR amplification, a systematic NGS enrichment analysis was performed to calculate the fold enrichment ratio relative to the corresponding TTM-001 or TTM-002 control, and the peptides comprised within the variants wre identified. The data for the TTM-001 matured capsid variants is provided in Table 20 and the data for the TTM-02 matured capsid variants is provided in Table 21.

As shown in Table 20, approximately 714 TTM-001 matured capsid variants demonstrated at least a 2-fold increase in expression relative to the non-matured TTM-001 control, and several variants demonstrated greater than a four-fold enrichment relative to the non-matured TTM-001 control. Also, across the peptides comprised within the TTM-001 matured capsid variants with the greatest fold-enrichment relative to the non-matured TTM-001 capsid in the brain, it was observed that the modifications in the variant sequences appeared in the region C-terminal to the SPH motif present within the capsid variant. This indicates that modifications that appeared to improve TTM-W01 capsid tropism in the CNS of mice were skewed to the C-terminal portion of the peptide insertion in loop TV of the sequence. Additionally, a number of these C-terminal modifications were the incorporation of an arginine (R) or leucine (L) residue.

TABLE 20
NGS fold-enrichment of TTM-001 matured AAV capsid variants in the brain of CD-1
Outbred mice

| | | Fold | | | Fold |
|---|---|---|---|---|---|
| | SEQ | enrichment | | SEQ | enrichment |
| | ID | over TTM- | | ID | over TTM- |
| Peptide Sequence | NO: | 001 | Peptide Sequence | NO: | 001 |
| KTINGSGSPHSLLWNQQT | 1008 | 7.983 | KTINGRPSPHVKAQNQQT | 1365 | 2.419 |
| KTINGSGSPHSKAQYYVT | 1009 | 6.283 | KTINGSGSPHSMFPNQQT | 1366 | 2.419 |
| KTINGSGSPHSKLRRQQT | 1010 | 6.231 | KTINGMKSPHSKAQNQQT | 1367 | 2.413 |
| KTINGSGSPHSIWQNQQT | 1011 | 5.883 | KTINGSGSPHSKVRAQQT | 1368 | 2.412 |
| KTINGSSSPHCTAQNQQT | 1012 | 5.607 | KGLVGSGSPHSKAQNQQT | 1369 | 2.412 |
| KTINGSGSPHSKAGCGQT | 1013 | 5.341 | KTINGSRSPHVRAQNQQT | 1370 | 2.412 |
| KSMNGSGSPHSRAQNQQT | 1014 | 5.145 | KTIRLRGSPHSKAQNQQT | 1371 | 2.408 |
| KTINGSGSPHSKRLRQQT | 1015 | 5.034 | KPRLGSGSPHSKAQNQQT | 1372 | 2.406 |
| KTINGSGSPHSLRRNQQT | 1016 | 4.985 | KTINGSGSPHSKAWYPQT | 1373 | 2.406 |
| KTINGSGSPHSRGRNQQT | 1017 | 4.961 | KTINGSCSPHVRAQNQQT | 1374 | 2.406 |
| KTINGSGSPHSEIVNQQT | 1018 | 4.931 | KTINGSGSPHSKRNVQQT | 1375 | 2.404 |
| KTINGSGSPHSSRRNQQT | 1019 | 4.920 | KTINGSGSPHSKRGLQQT | 1376 | 2.402 |
| KTINGSGSPHCLLQNQQT | 1020 | 4.898 | KTINGSGSPHSKRLAQQT | 1377 | 2.397 |
| KTIMRVGSPHSKAQNQQT | 1021 | 4.875 | KTINGSGSPHSKVTRQQT | 1378 | 2.397 |
| KTINGSGSPHSKAFRLQT | 1022 | 4.849 | KTINGSGSPHTWLQNQQT | 1379 | 2.397 |
| KTINGSGSPHCLAQNQQT | 1023 | 4.847 | KTINGSGSPHSKQRSQQT | 1380 | 2.395 |
| KTINGSCSPHRKAQNQQT | 1024 | 4.801 | KTINGSGSPHSKAQRCST | 1381 | 2.393 |
| KTINGSGSPHFLRQNQQT | 1025 | 4.777 | KTINGSPSPHYLAQNQQT | 1382 | 2.391 |
| KTINGSGSPHSLRENQQT | 1026 | 4.765 | KTINGRRSPHLKAQNQQT | 1383 | 2.391 |
| KTINGSGSPHSYLRNQQT | 1027 | 4.566 | KTINGSGSPHWSLQNQQT | 1384 | 2.389 |
| KTINGSGSPHCSLQNQQT | 1028 | 4.540 | KTINGVTSPHWKAQNQQT | 1385 | 2.387 |
| KTINGSGSPHVLWQNQQT | 1029 | 4.533 | KTINGSGSPHSKAQTTRT | 1386 | 2.385 |
| KTINGSGSPHSKWLLQQT | 1030 | 4.521 | KTINGSGSPHSKAQLFKT | 1387 | 2.385 |
| KTINGSGSPHSLWSNQQT | 1031 | 4.467 | KTINGSGSPHSKARSYQT | 1388 | 2.382 |
| KTINGSGSPHSKRRLQQT | 1032 | 4.451 | KTINGSGSPHSKLSRQQT | 1389 | 2.380 |
| KTINGSGSPHSVYLNQQT | 1033 | 4.426 | KTINGSGSPHLVFQNQQT | 1390 | 2.376 |
| KTINGSGSPHSLWLNQQT | 1034 | 4.412 | KTINGSGSPHSKAQLVKT | 1391 | 2.376 |
| KTINGSGSPHSKAQRKLT | 1035 | 4.339 | KTINGSGSPHSKAQTGRT | 1392 | 2.371 |
| KTINGSGSPHSKALRRQT | 1036 | 4.330 | KTINGSGSPHSKARYSQT | 1393 | 2.369 |
| KTINGSGSPHSKAQRLRT | 1037 | 4.322 | KTINGRHSPHLKAQNQQT | 1394 | 2.369 |
| KYLSGSGSPHSKAQNQQT | 1038 | 4.264 | KTINGSGSPHSFARNQQT | 1395 | 2.367 |
| KTINGSGSPHSKAQRRLT | 1039 | 4.227 | KTINGSGSPHSSCQNQQT | 1396 | 2.365 |
| KTINGSGSPHSKARRQQT | 1040 | 4.218 | KTINGSGSPHSLFANQQT | 1397 | 2.365 |
| KTINGSGSPHSKARRLQT | 1041 | 4.210 | KTINGSGSPHSKRLTQQT | 1398 | 2.363 |
| KTINGSGSPHSKSRRQQT | 1042 | 4.175 | KTINGSGSPHSKAQTART | 1399 | 2.363 |
| KTINGLLSPHWKAQNQQT | 1043 | 4.173 | KTINGSGSPHFLNQNQQT | 1400 | 2.359 |
| KTINGSGSPHSKARLRQT | 1044 | 4.155 | KTINGSGSPHSKAQLILT | 1401 | 2.358 |
| KTINGSGSPHSKASKRQT | 1045 | 4.117 | KEVGGSGSPHSKAQNQQT | 1402 | 2.358 |
| KTINGSGSPHVRRQNQQT | 1046 | 4.114 | KTINGSRSPHIRAQNQQT | 1403 | 2.356 |
| KTINGSGSPHSKAQLYRT | 1047 | 4.108 | KTINGSGSPHSSWLNQQT | 1404 | 2.354 |
| KGLSGSGSPHSKAQNQQT | 1048 | 4.056 | KTINGSMSPHLYAQNQQT | 1405 | 2.354 |
| KTINGSGSPHSLERNQQT | 1049 | 4.037 | KTINGMSSPHRKAQNQQT | 1406 | 2.352 |
| KTINGSGSPHSKAQLTVT | 1050 | 4.026 | KTINGSGSPHSKPRPQQT | 1407 | 2.352 |
| KTINGSRSPHTRAQNQQT | 1051 | 3.989 | KTINGSGSPHSGLWNQQT | 1408 | 2.350 |
| KTINGSGSPHSKAKLRQT | 1052 | 3.976 | KTINGSGSPHRWAQNQQT | 1409 | 2.350 |
| KTINGSGSPHSKLIRQQT | 1053 | 3.968 | KTINGSGSPHSKIRLQQT | 1410 | 2.348 |
| KTINGSGSPHSKALRFQT | 1054 | 3.894 | KTINGSGSPHSKESCQQT | 1411 | 2.348 |
| KTINGSGSPHSKRTFQQT | 1055 | 3.879 | KTINGSGSPHSKSCAQQT | 1412 | 2.348 |
| KTINGSGSPHSKAQKRLT | 1056 | 3.872 | KTINGSGSPHSKRLMQQT | 1413 | 2.345 |
| KTINGSGSPHLWSQNQQT | 1057 | 3.857 | KTSRCSGSPHSKAQNQQT | 1414 | 2.345 |
| KTINGSGSPHLLWQNQQT | 1058 | 3.855 | KTINGSGSPHSFLLNQQT | 1415 | 2.341 |
| KTINGSGSPHSRLRNQQT | 1059 | 3.851 | KTINGSGSPHCSAQNQQT | 1416 | 2.339 |
| KTINGSGSPHSKRAAQQT | 1060 | 3.838 | KTINGSGSPHSKAQPSKT | 1417 | 2.339 |
| KTINGSGSPHSKRSWQQT | 1061 | 3.838 | KTINGSGSPHSYVRNQQT | 1418 | 2.339 |
| KTINGSGSPHSKAQLRRT | 1062 | 3.825 | KTINGSGSPHSKAQQSRT | 1419 | 2.337 |
| KTINGSGSPHYLVQNQQT | 1063 | 3.819 | KTINGSGSPHSFVVNQQT | 1420 | 2.335 |
| KTINGSGSPHSKLFRQQT | 1064 | 3.806 | KTINGERSPHSKAQNQQT | 1421 | 2.333 |
| KTINGSGSPHSKRAMQQT | 1065 | 3.801 | KTINGSGSPHSKWLVQQT | 1422 | 2.333 |
| KTINGSGSPHSKTLRQQT | 1066 | 3.788 | KTINGSGSPHSKRTAQQT | 1423 | 2.331 |
| KTINGSGSPHSRSRNQQT | 1067 | 3.784 | KTINGLESPHRKAQNQQT | 1424 | 2.331 |
| KTINGSGSPHRRRQNQQT | 1068 | 3.754 | KTINTIESPHSKAQNQQT | 1425 | 2.331 |
| KTINGSGSPHSKTCLQQT | 1069 | 3.717 | KRLFGSGSPHSKAQNQQT | 1426 | 2.330 |
| KTINGSGSPHSKSRWQQT | 1070 | 3.698 | KTINGSGSPHSKAPNHLT | 1427 | 2.324 |
| KTINGSGSPHRFRQNQQT | 1071 | 3.698 | KTINGSGSPHLFRQNQQT | 1428 | 2.324 |
| KTINGLRSPHRKAQNQQT | 1072 | 3.676 | KTINGSGSPHSKASRHQT | 1429 | 2.324 |
| KTRSRSGSPHSKAQNQQT | 1073 | 3.669 | KTINGSGSPHSKLSWQQT | 1430 | 2.322 |
| KTINGSGSPHSKAQLVVT | 1074 | 3.654 | KETAGSGSPHSKAQNQQT | 1431 | 2.320 |
| KTINGSGSPHSRKLNQQT | 1075 | 3.646 | KTINGHRSPHLKAQNQQT | 1432 | 2.320 |
| KTINGSGSPHSLLCNQQT | 1076 | 3.644 | KTINGSGSPHSKGCLQQT | 1433 | 2.320 |
| KTINGKRSPHSKAQNQQT | 1077 | 3.611 | KTINGSGSPHSKAQVLIT | 1434 | 2.317 |
| KTINGSRSPHLFAQNQQT | 1078 | 3.601 | KTINGSGSPHSKLRSQQT | 1435 | 2.309 |
| KSINGSGSPHSKAHDQQT | 1079 | 3.592 | KTINGTLSPHRKAQNQQT | 1436 | 2.307 |
| KTINGSGSPHSKAQRSRT | 1080 | 3.585 | KTINGSGSPHSTWINQQT | 1437 | 2.307 |
| KTINGSGSPHSTWLNQQT | 1081 | 3.583 | KTINGSGSPHSKAQCRLT | 1438 | 2.304 |
| KTINGSGSPHSKASRRQT | 1082 | 3.577 | KTINNLRSPHSKAQNQQT | 1439 | 2.302 |
| KTINGSGSPHSKRSMQQT | 1083 | 3.561 | KTINGSGSPHSKARANQT | 1440 | 2.300 |
| KTINGSGSPHSCLQNQQT | 1084 | 3.559 | KTINGRQSPHTKAQNQQT | 1441 | 2.294 |
| KTINGSGSPHSKRLWQQT | 1085 | 3.529 | KTINSARSPHSKAQNQQT | 1442 | 2.292 |
| KTINGSGSPHSWLSNQQT | 1086 | 3.495 | KTINGCSSPHRKAQNQQT | 1443 | 2.291 |
| KTINGSGSPHLRRQNQQT | 1087 | 3.493 | KTINGSVSPHEMAQNQQT | 1444 | 2.287 |
| KTINGSGSPHSKARRSQT | 1088 | 3.493 | KTINGSGSPHSLCQNQQT | 1445 | 2.285 |
| KTINGSGSPHSKHLRQQT | 1089 | 3.438 | KTINGSLSPHLFAQNQQT | 1446 | 2.285 |
| KTINGSGSPHSCSQNQQT | 1090 | 3.428 | KTINGSGSPHSKACPLQT | 1447 | 2.285 |
| KTINGSGSPHSKSFRQQT | 1091 | 3.426 | KTINGRTSPHRKAQNQQT | 1448 | 2.285 |
| KTINGSGSPHLCLQNQQT | 1092 | 3.425 | KTINGSGSPHSKRATQQT | 1449 | 2.283 |
| KTINGSGSPHSKAQTSRT | 1093 | 3.421 | KTINGSGSPHSKARIMQT | 1450 | 2.283 |
| KTINGSGSPHSLCSNQQT | 1094 | 3.413 | KTINGSGSPHVTWQNQQT | 1451 | 2.281 |
| KTINGSRSPHLRAQNQQT | 1095 | 3.410 | KTINGSGSPHSKRLPQQT | 1452 | 2.279 |
| KTINGSGSPHSKAQVSKT | 1096 | 3.406 | KTINGSGSPHSKAQGERT | 1453 | 2.279 |
| KTINGSGSPHSKAQRHVT | 1097 | 3.404 | KTINGSGSPHLYGQNQQT | 1454 | 2.277 |
| KTINGSSSPHLCAQNQQT | 1098 | 3.402 | KTINGSGSPHLSCQNQQT | 1455 | 2.277 |
| KTINGSGSPHSFLRNQQT | 1099 | 3.384 | KTINGSGSPHSKAQFTLT | 1456 | 2.277 |
| KTINGSGSPHSFVLNQQT | 1100 | 3.382 | KTINGSRSPHFKAQNQQT | 1457 | 2.277 |
| KTINGSGSPHSKMRAQQT | 1101 | 3.382 | KTINGRPSPHSKAQNQQT | 1458 | 2.276 |
| KTINGSGSPHRPRQNQQT | 1102 | 3.380 | KTINGFSSPHRKAQNQQT | 1459 | 2.276 |
| KTINGSGSPHSKCLLQQT | 1103 | 3.374 | KTINGRASPHVKAQNQQT | 1460 | 2.272 |
| KTINGSGSPHSKAQSRRT | 1104 | 3.372 | KTINGSGSPHSKAQNEVH | 1461 | 2.272 |
| KTINGSGSPHSRWQNQQT | 1105 | 3.372 | KTINGSGSPHSKRSLQQT | 1462 | 2.270 |
| KYSVGSGSPHSKAQNQQT | 1106 | 3.365 | KTINGSGSPHRSRQNQQT | 1463 | 2.270 |
| KTINGSGSPHSKRELQQT | 1107 | 3.359 | KPPTGSGSPHSKAQNQQT | 1464 | 2.270 |
| KTINGSGSPHSLFLNQQT | 1108 | 3.358 | KTINGSGSPHSKAARTQT | 1465 | 2.266 |
| KTINGSGSPHSKAYLRQT | 1109 | 3.356 | KTINGSGSPHSSWANQQT | 1466 | 2.264 |
| KTINGSGSPHSKRNGQQT | 1110 | 3.350 | KTINGSGSPHSKAQRHAT | 1467 | 2.264 |
| KTINGSGSPHTRRQNQQT | 1111 | 3.350 | KTINSPRSPHSKAQNQQT | 1468 | 2.264 |
| KTINGSGSPHSKPRLQQT | 1112 | 3.337 | KTINGSGSPHSKSERQQT | 1469 | 2.263 |
| KTINFLRSPHSKAQNQQT | 1113 | 3.331 | KTINGSGSPHALFQNQQT | 1470 | 2.261 |
| KTINGSGSPHLLCQNQQT | 1114 | 3.328 | KTINGSGSPHSKAQCYVT | 1471 | 2.261 |
| KTINGSGSPHSKARIVQT | 1115 | 3.287 | KTINGVASPHRKAQNQQT | 1472 | 2.261 |
| KTINGSKSPHFKAQNQQT | 1116 | 3.285 | KTINGSGSPHSALWNQQT | 1473 | 2.261 |
| KTINGSGSPHSKAQIRLT | 1117 | 3.279 | KTINGSGSPHSKSVRQQT | 1474 | 2.259 |
| KTINGSSSPHWVAQNQQT | 1118 | 3.277 | KTINGSGSPHSHMENQQT | 1475 | 2.259 |
| KTINGSGSPHSKATRRQT | 1119 | 3.277 | KTINGSGTPHSKAQNQQT | 1476 | 2.259 |
| KTINGSLSPHSCAQNQQT | 1120 | 3.268 | KTINGSGSPHSKTGRQQT | 1477 | 2.259 |
| KTINGSGSPHSLYLNQQT | 1121 | 3.264 | KTINGSGSPHSKAQANRT | 1478 | 2.255 |
| KTINGSGSPHSKVGRQQT | 1122 | 3.255 | KTINGSGSPHSKARFSQT | 1479 | 2.253 |
| KTINGSGSPHSRRLNQQT | 1123 | 3.251 | KYLLGSGSPHSKAQNQQT | 1480 | 2.253 |
| KTINGSGSPHSKAQHSRT | 1124 | 3.227 | KTINGSGSPHCSSQNQQT | 1481 | 2.253 |
| KTINGSGSPHSKAFPRQT | 1125 | 3.220 | KTINGSMSPHRKAQNQQT | 1482 | 2.251 |
| KTINGSPSPHRRAQNQQT | 1126 | 3.216 | KTINGNLSPHRKAQNQQT | 1483 | 2.250 |
| KTINGSGSPHSKRNLQQT | 1127 | 3.210 | KEVAGSGSPHSKAQNQQT | 1484 | 2.250 |
| KTINGSGSPHSKPTRQQT | 1128 | 3.201 | KTINLSRSPHSKAQNQQT | 1485 | 2.246 |
| KTINGSGSPHSKLWLQQT | 1129 | 3.199 | KTINGSGSPHSKARQQQT | 1486 | 2.244 |
| KTINGSGSPHWLAQNQQT | 1130 | 3.192 | KTINGTPSPHRKAQNQQT | 1487 | 2.244 |
| KTINGSGSPHRTRQNQQT | 1131 | 3.190 | KTINGSGSPHSKEKLQQT | 1488 | 2.244 |
| KTINGSGSPHSKLNKQQT | 1132 | 3.181 | KTINGSGSPHSKAWLLQT | 1489 | 2.240 |
| KTINGSGSPHSSLWNQQT | 1133 | 3.179 | KTINGLRSPHSKAQNQQT | 1490 | 2.238 |
| KTINGSGSPHSKAQITLT | 1134 | 3.177 | KTINGRLSPHRKAQNQQT | 1491 | 2.238 |
| KTINGSGSPHSKFLFQQT | 1135 | 3.173 | KTINGSPSPHLFAQNQQT | 1492 | 2.238 |
| KTINGSGSPHSKRTPQQT | 1136 | 3.169 | KDLRGSGSPHSKAQNQQT | 1493 | 2.238 |
| KTINGSGSPHSKAQNSRR | 1137 | 3.168 | KTINGSGSPHSKAQLAKT | 1494 | 2.238 |
| KTINGSGSPHSRLKNQQT | 1138 | 3.156 | KTINGSGSPHSKPRSQQT | 1495 | 2.235 |
| KTINGSGSPHSCLLNQQT | 1139 | 3.127 | KTINGSGSPHSKKMSQQT | 1496 | 2.235 |
| KTINGSGSPHTLYQNQQT | 1140 | 3.117 | KTINGSGSPHSKAQLIVT | 1497 | 2.235 |
| KTINGSGSPHSKYPSQQT | 1141 | 3.114 | KTINGSGSPHSKARFTQT | 1498 | 2.233 |
| KTINGSGSPHSKLRNQQT | 1142 | 3.112 | KTINGSGSPHPLFQNQQT | 1499 | 2.233 |
| KTINGSGSPHLNWQNQQT | 1143 | 3.112 | KTINGSGSPHSKAQRGMT | 1500 | 2.231 |
| KTINGVVSPHRKAQNQQT | 1144 | 3.106 | KTINGSGSPHSKAQNLRR | 1501 | 2.231 |
| KTINGSGSPHSYRPNQQT | 1145 | 3.095 | KTINGSGSPHSKAQFRVT | 1502 | 2.231 |
| KTINGSTSPHRRAQNQQT | 1146 | 3.089 | KTINGSGSPHSKAFVRQT | 1503 | 2.225 |
| KTINGSCSPHPLAQNQQT | 1147 | 3.086 | KTINGSGSPHSKARLTQT | 1504 | 2.223 |
| KTINGSGSPHSKAFARQT | 1148 | 3.082 | KTINGSGSPHRFKQNQQT | 1505 | 2.223 |
| KTINGSGSPHSKALRYQT | 1149 | 3.076 | KTINGSGSPHSKEETQQT | 1506 | 2.223 |
| KTINGSKSPHRLAQNQQT | 1150 | 3.073 | KTINGSGSPHSKTRAQQT | 1507 | 2.223 |
| KTINMRVSPHSKAQNQQT | 1151 | 3.073 | KTINGSGSPHSVSWNQQT | 1508 | 2.223 |
| KTINGSGSPHMYLQNQQT | 1152 | 3.061 | KTINGSGSPHTKWQNQQT | 1509 | 2.222 |
| KTINGSGSPHSKLARQQT | 1153 | 3.054 | KTINGSNSPHRKAQNQQT | 1510 | 2.218 |
| KTINGSGSPHSKARPYQT | 1554 | 3.050 | KTINGSGSPHSKAQNKRS | 1511 | 2.214 |
| KTINGSGSPHSKRVPQQT | 1155 | 3.048 | KTINGSGSPHSTRQNQQT | 1512 | 2.212 |
| KTINGSGSPHLSWQNQQT | 1156 | 3.047 | KTINGTRSPHTKAQNQQT | 1513 | 2.203 |
| KTINGRSSPHGKAQNQQT | 1157 | 3.035 | KTINGSGSPHVLFQNQQT | 1514 | 2.203 |
| KTINGSGSPHLWTQNQQT | 1158 | 3.034 | KTINGSVSPHYLAQNQQT | 1515 | 2.203 |
| KTINGLLSPHRKAQNQQT | 1159 | 3.026 | KTINGALSPHRKAQNQQT | 1516 | 2.203 |
| KTINGSGSPHRLRQNQQT | 1160 | 2.998 | KTINGSGSPHSKARLYQT | 1517 | 2.201 |
| KTINGSCSPHSGAQNQQT | 1161 | 2.994 | KTINGSGSPHEHNQNQQT | 1518 | 2.199 |
| KTINGSGSPHSKAQRRST | 1162 | 2.993 | KTINGVLSPHWKAQNQQT | 1519 | 2.199 |
| KTINGSGSPHSKLCSQQT | 1163 | 2.989 | KTINGSGSPHSKASRQQT | 1520 | 2.197 |
| KTINGSGSPHSKAQLLKT | 1164 | 2.985 | KTINGSGSPHSKRSFQQT | 1521 | 2.197 |
| KTINGRKSPHSKAQNQQT | 1165 | 2.985 | KTINGSGSPHSKRVSQQT | 1522 | 2.196 |
| KTINGSGSPHLLYQNQQT | 1166 | 2.983 | KTINGSGSPHSYSRNQQT | 1523 | 2.196 |
| KTINGSGSPHSKLLRQQT | 1167 | 2.981 | KTINGSGSPHSTVWNQQT | 1524 | 2.196 |
| KTINGSGSPHSLRHNQQT | 1168 | 2.980 | KTINGSGSPHSVLFNQQT | 1525 | 2.194 |
| KTINGSGSPHSSKRNQQT | 1169 | 2.978 | KTINGPLSPHCKAQNQQT | 1526 | 2.194 |
| KTINGSGSPHSKARSRQT | 1170 | 2.972 | KTINGSGSPHSKRVGQQT | 1527 | 2.190 |
| KTINGRSSPHRKAQNQQT | 1171 | 2.965 | KTINGSGSPHSKLWSQQT | 1528 | 2.190 |
| KTINGSKSPHRTAQNQQT | 1172 | 2.950 | KTINGSGSPHSKAQGVRT | 1529 | 2.188 |
| KTINGMRSPHVKAQNQQT | 1173 | 2.937 | KTINGSVSPHRRAQNQQT | 1530 | 2.186 |
| KTINGSGSPHSKRMSQQT | 1174 | 2.931 | KTINGSGSPHLRFQNQQT | 1531 | 2.186 |
| KTINGSGSPHSKVPKQQT | 1175 | 2.924 | KTINGSASPHVFAQNQQT | 1532 | 2.186 |
| KTINLIRSPHSKAQNQQT | 1176 | 2.920 | KTWVRSGSPHSKAQNQQT | 1533 | 2.186 |
| KTINGSGSPHPFLQNQQT | 1177 | 2.916 | KTINGSGSPHSKARMQQT | 1534 | 2.184 |
| KTINGSGSPHSKARLWQT | 1178 | 2.914 | KTINGSGSPHSKASRGQT | 1535 | 2.182 |
| KTINGSGSPHSRTRNQQT | 1179 | 2.912 | KTINGSGSPHSKAQVCLT | 1536 | 2.182 |
| KTINGSGSPHSKRSNQQT | 1180 | 2.886 | KTINGSGSPHSKARGVQT | 1537 | 2.181 |
| KTINGSLSPHSWAQNQQT | 1181 | 2.885 | KTINGSGSPHGLWQNQQT | 1538 | 2.181 |
| KTINGSRSPHYKAQNQQT | 1182 | 2.879 | KTINGSGSPHSKAQVWFT | 1539 | 2.181 |
| KTINRHSSPHSKAQNQQT | 1183 | 2.877 | KTINGSGSPHSKAQVTLT | 1540 | 2.179 |
| KTINGSGSPHSKRRNQQT | 1184 | 2.877 | KTINGSGSPHSKAQLRIT | 1541 | 2.179 |
| KTINGSGSPHSKAKHLQT | 1185 | 2.870 | KDSLGSGSPHSKAQNQQT | 1542 | 2.175 |
| KTINGSGSPHSKRTYQQT | 1186 | 2.870 | KTINGSGSPHSKRASQQT | 1543 | 2.173 |
| KTINGVLSPHRKAQNQQT | 1187 | 2.868 | KTINGSGSPHSKRINQQT | 1544 | 2.173 |
| KTINGSGSPHSFITNQQT | 1188 | 2.868 | KTINGSGSPHSKASKNQT | 1545 | 2.171 |
| KTINGSGSPHSTRLNQQT | 1189 | 2.860 | KTINGSGSPHSKAQLPWT | 1546 | 2.169 |
| KTINGSGSPHSKRTSQQT | 1190 | 2.857 | KTINGSGSPHSKLTRQQT | 1547 | 2.169 |
| KTINGSGSPHSRRSNQQT | 1191 | 2.851 | KTINGSGSPHSKINRQQT | 1548 | 2.169 |
| KTINGHLSPHRKAQNQQT | 1192 | 2.851 | KTINRVISPHSKAQNQQT | 1549 | 2.169 |
| KTINGSGSPHSKAQFSRT | 1193 | 2.847 | KTINGSGSPHTLWQNQQT | 1550 | 2.168 |
| KTINGSGSPHSKAQTERT | 1194 | 2.847 | KTINGSGSPHSRRQNQQT | 1551 | 2.166 |
| KTINGSGSPHSKPLRQQT | 1195 | 2.844 | KTINGSGSPHSKGGRQQT | 1552 | 2.164 |
| KTINGSGSPHSKASCRQT | 1196 | 2.840 | KTINGSESPHDSAQNQQT | 1553 | 2.164 |
| KTINGSGSPHSKILWQQT | 1197 | 2.838 | KTINGSGSPHSRPRNQQT | 1554 | 2.164 |
| KTINGSGSPHSKALKRQT | 1198 | 2.836 | KTINGSGSPHSRKQNQQT | 1555 | 2.162 |
| KTINGSGSPHSKAHRSQT | 1199 | 2.819 | KTINGSGSPHSKAQEELT | 1556 | 2.162 |
| KTINGSGSPHSMLYNQQT | 1200 | 2.808 | KTINGWRSPHSKAQNQQT | 1557 | 2.160 |
| KTINGSGSPHSKCTLQQT | 120 | 2.808 | KTINGSGSPHSLLYNQQT | 1558 | 2.158 |
| KTINGSGSPHSKAQNRMR | 120 | 2.804 | KTINGSGSPHSERLNQQT | 1559 | 2.158 |
| KTINGSGSPHSKLVRQQT | 1203 | 2.801 | KTINGSGSPHSKAQFLRT | 1560 | 2.156 |
| KTINGSGSPHSKRILQQT | 1204 | 2.801 | KTINGSGSPHSKQSRQQT | 1561 | 2.156 |
| KTINGSGSPHSKAQWERT | 1205 | 2.795 | KTINGSRSPHSKAQNRQT | 1562 | 2.155 |
| KTINGSGSPHSLTCNQQT | 1206 | 2.795 | KTINGRPSPHIKAQNQQT | 1563 | 2.155 |
| KTINGIRSPHTKAQNQQT | 1207 | 2.793 | KTINGSGSPHSKRLVQQT | 1564 | 2.151 |
| KTINGSGSPHSKAQRWLT | 1208 | 2.788 | KGHEGSGSPHSKAQNQQT | 1565 | 2.151 |
| KTINGSGSPHSKAQLSIT | 1209 | 2.784 | KTINGSGSPHSKAQKRST | 1566 | 2.151 |
| KTINGSGSPHIYRQNQQT | 1210 | 2.782 | KTINGSGSPHSYLLNQQT | 1567 | 2.147 |
| KTINGSGSPHSLRSNQQT | 1211 | 2.778 | KTINGSGSPHSKPRGQQT | 1568 | 2.147 |
| KTINGSGSPHSKVKPQQT | 1212 | 2.778 | KTINGSGSPHSKTRLQQT | 1569 | 2.145 |
| KTINGSGSPHSKATRHQT | 1213 | 2.777 | KTINGSGSPHSKSHRQQT | 1570 | 2.145 |
| KTINGSLSPHLCAQNQQT | 1214 | 2.775 | KEIKGSGSPHSKAQNQQT | 1571 | 2.140 |
| KTINGSGSPHSKACASQT | 1215 | 2.775 | KTINGSGSPHSKARGIQT | 1572 | 2.140 |
| KWSPGSGSPHSKAQNQQT | 1216 | 2.764 | KTINGYRSPHSKAQNQQT | 1573 | 2.140 |
| KTINGYLSPHRKAQNQQT | 1217 | 2.762 | KTINGSGSPHSKLWTQQT | 1574 | 2.140 |
| KTINGSGSPHSKVIRQQT | 1218 | 2.760 | KTINGSGSPHSKPWLQQT | 1575 | 2.138 |
| KTINGSGSPHELLQNQQT | 1219 | 2.758 | KTINGSGSPHWSVQNQQT | 1576 | 2.138 |
| KTINGSGSPHSKARSKQT | 1220 | 2.758 | KTINGSGSPHSKVARQQT | 1577 | 2.136 |
| KTINGVPSPHWKAQNQQT | 1221 | 2.758 | KTINGSGSPHTLFQNQQT | 1578 | 2.136 |
| KTINGSGSPHSKATRNQT | 1222 | 2.756 | KTINGSCSPHLAAQNQQT | 1579 | 2.134 |
| KTINGSGSPHSKACSAQT | 1223 | 2.754 | KTINGSGSPHSKTSRQQT | 1580 | 2.132 |
| KTINGSGSPHSKARYVQT | 1224 | 2.749 | KTINGSGSPHSKAQNARH | 1581 | 2.127 |
| KTINGSRSPHARAQNQQT | 1225 | 2.745 | KTINGSGSPHSKAQLKLT | 1582 | 2.125 |
| KTINGSGSPHSKAQHLRT | 1226 | 2.741 | KTINGSGSPHSKAQNWRT | 1583 | 2.125 |
| KTINGSGSPHSKAKSRQT | 1227 | 2.739 | KTINGSGSPHELPQNQQT | 1584 | 2.123 |
| KTINGSGSPHSKIGRQQT | 1228 | 2.739 | KTINGSGSPHSKNVRQQT | 1585 | 2.123 |
| KTINGLASPHRKAQNQQT | 1229 | 2.737 | KTINGSGSPHEMRQNQQT | 1586 | 2.123 |
| KTINGSGSPHSKARTRQT | 1230 | 2.737 | KTINGSGSPHGWAQNQQT | 1587 | 2.121 |
| KTINGSGSPHSKSIRQQT | 1231 | 2.728 | KTINGSGSPHFHLQNQQT | 1588 | 2.121 |
| KTINGSGSPHSKRLYQQT | 1232 | 2.721 | KTINGSASPHWSAQNQQT | 1589 | 2.121 |
| KTINGLPSPHRKAQNQQT | 1233 | 2.719 | KTINGSSSPHSWAQNQQT | 1590 | 2.119 |
| KTINGSLSPHRRAQNQQT | 1234 | 2.717 | KTINGSGSPHSKAHRQQT | 1591 | 2.117 |
| KTINGKTSPHGKAQNQQT | 1235 | 2.717 | KTINGSGSPHSKQRVQQT | 1592 | 2.117 |
| KTINGSRSPHRLAQNQQT | 1236 | 2.698 | KTLRRSGSPHSKAQNQQT | 1593 | 2.117 |
| KTINGSGSPHSLTWNQQT | 1237 | 2.698 | KTINGSGSPHSKGVRQQT | 1594 | 2.115 |
| KTINGSKSPHRKAQNQQT | 1238 | 2.696 | KTINGSLSPHTWAQNQQT | 1595 | 2.115 |
| KTINGSGSPHSKAQLRKT | 1239 | 2.689 | KTINGSGSPHSKRALQQT | 1596 | 2.114 |
| KTINGSGSPHSKSRHQQT | 1240 | 2.685 | KTINGSGSPHCLSQNQQT | 1597 | 2.114 |
| KTINRRLSPHSKAQNQQT | 1241 | 2.678 | KTINGSGSPHSKAQSLKT | 1598 | 2.110 |
| KTINGSGSPHSRRVNQQT | 1242 | 2.676 | KTINGSGSPHSFVRNQQT | 1599 | 2.110 |
| KTINGSGSPHSHWQNQQT | 1243 | 2.676 | KTINGSGSPHSIFSNQQT | 1600 | 2.110 |
| KTTHCSGSPHSKAQNQQT | 1244 | 2.672 | KTINGSGSPHSKVSRQQT | 160 | 2.108 |
| KTINGSGSPHSWLQNQQT | 1245 | 2.665 | KTINGSGSPHSKARNKQT | 1602 | 2.108 |
| KTINGSTSPHYLAQNQQT | 1246 | 2.665 | KTINASGSPHSKAQGQQT | 1603 | 2.108 |
| KTINGLTSPHRKAQNQQT | 1247 | 2.663 | KTINGSGSPHSKLRMQQT | 1604 | 2.106 |
| KTINGSGSPHSKRLLQQT | 1248 | 2.659 | KTINGSWSPHMLAQNQQT | 1605 | 2.106 |
| KTINGSGSPHSKLCVQQT | 1249 | 2.659 | KTINGSGSPHSLFPNQQT | 1606 | 2.106 |
| KTINGELSPHRKAQNQQT | 1250 | 2.654 | KPPLGSGSPHSKAQNQQT | 1607 | 2.102 |
| KTINGSGSPHSKMRPQQT | 1251 | 2.652 | KTINGIASPHRKAQNQQT | 1608 | 2.099 |
| KTINGSGSPHSKQTRQQT | 1252 | 2.650 | KTINGSCSPHSLAQNQQT | 1609 | 2.099 |
| KTINGSGSPHSYLINQQT | 1253 | 2.650 | KTINGRLSPHFKAQNQQT | 1610 | 2.097 |
| KTINGSGSPHSKALRSQT | 1254 | 2.648 | KTINGSGSPHSKARMTQT | 1611 | 2.091 |
| KTINGMLSPHRKAQNQQT | 1255 | 2.646 | KTINGSGSPHSKARLQQT | 1612 | 2.089 |
| KTINGSGSPHSKCLTQQT | 1256 | 2.644 | KTINGSGSPHSKWVSQQT | 1613 | 2.089 |
| KTINGSGSPHSKAQLTLT | 1257 | 2.641 | KTINGSGSPHSKKVSQQT | 1614 | 2.088 |
| KTINGHSSPHRKAQNQQT | 1258 | 2.639 | KTINGSGSPHSKAQSYRT | 1615 | 2.088 |
| KTINGSGSPHLTWQNQQT | 1259 | 2.637 | KAFNGSGSPHSKAPNQQT | 1616 | 2.088 |
| KTINGSGSPHSKAQYCLT | 1260 | 2.628 | KTINGSGSPHSKAQYRLT | 1617 | 2.088 |
| KTINGSGSPHSELVNQQT | 1261 | 2.624 | KTINGSWSPHLVAQNQQT | 1618 | 2.084 |
| KTINMSRSPHSKAQNQQT | 1262 | 2.622 | KTINGSGSPHSWTQNQQT | 1619 | 2.084 |
| KTINGSGSPHSKAQLHRT | 1263 | 2.618 | KTINGSGSPHSKAQSHRT | 1620 | 2.084 |
| KTINGSGSPHLYMQNQQT | 1264 | 2.615 | KGINGSGSPHGKAQNQQT | 1621 | 2.084 |
| KTINGSRSPHRRAQNQQT | 1265 | 2.615 | KTINGSGSPHSKAQNRKL | 1622 | 2.084 |
| KTINGSGSPHSKAQNRRS | 1266 | 2.613 | KTINGRYSPHSKAQNQQT | 1623 | 2.080 |
| KTINLRFSPHSKAQNQQT | 1267 | 2.611 | KTINGSGSPHSKGRSQQT | 1624 | 2.080 |
| KTINGSGSPHSKAQRLWT | 1268 | 2.611 | KTINGSGSPHCVAQNQQT | 1625 | 2.080 |
| KTINGSGSPHSKGRAQQT | 1269 | 2.607 | KTINGSGSPHSKIRPQQT | 1626 | 2.080 |
| KTINGSGSPHSLSCNQQT | 1270 | 2.605 | KTINGSGSPHSKAQSSKT | 1627 | 2.078 |
| KTINGLVSPHCKAQNQQT | 1271 | 2.605 | KTINGSGSPHSKRPFQQT | 1628 | 2.076 |
| KTINGSSSPHLWAQNQQT | 1272 | 2.605 | KTINGSSSPHCLAQNQQT | 1629 | 2.074 |
| KTINGSGSPHSKAHRLQT | 1273 | 2.603 | KTINGTRSPHAKAQNQQT | 1630 | 2.071 |
| KTINGSGSPHPYAQNQQT | 1274 | 2.598 | KTINGSGSPHLLFQNQQT | 1631 | 2.069 |
| KTINGSGSPHSTRPNQQT | 1275 | 2.598 | KTINGRRSPHTKAQNQQT | 1632 | 2.069 |
| KTINGRSSPHPKAQNQQT | 1276 | 2.596 | KTINGSGSPHSKASKQQT | 1633 | 2.069 |
| KTINGSGSPHSKAQSWRT | 1277 | 2.596 | KTINGSGSPHSKAQLGRT | 1634 | 2.069 |
| KTINGQRSPHVKAQNQQT | 1278 | 2.596 | KTINGSGSPHSVELNQQT | 1635 | 2.069 |
| KTINGSGSPHSKAQFVRT | 1279 | 2.596 | KTINGSGSPHSKSARQQT | 1636 | 2.067 |
| KTINGSGSPHSKCINQQT | 1280 | 2.594 | KTINGSGSPHSKLRLQQT | 1637 | 2.065 |
| KTINGSGSPHSSLCNQQT | 1281 | 2.592 | KTRKSSGSPHSKAQNQQT | 1638 | 2.065 |
| KTINGQRSPHSKAQNQQT | 1282 | 2.590 | KTINGERSPHLKAQNQQT | 1639 | 2.063 |
| KTINGSGSPHSLSWNQQT | 1283 | 2.588 | KTINGSGSPHSKRSIQQT | 1640 | 2.063 |
| KTINGSGSPHSSRKNQQT | 1284 | 2.585 | KTINGSGSPHSKGRIQQT | 1641 | 2.061 |
| KTINGSGSPHSKRTLQQT | 1285 | 2.583 | KTINGSRSPHRPAQNQQT | 1642 | 2.061 |
| KTINGSLSPHCLAQNQQT | 1286 | 2.577 | KTINGSGSPHSKLRPQQT | 1643 | 2.060 |
| KTINGSGSPHSKAQSSRT | 1287 | 2.575 | KTINGSGSPHMYAQNQQT | 1644 | 2.060 |
| KTINGSGSPHLKRQNQQT | 1288 | 2.564 | KTINGRTSPHAKAQNQQT | 1645 | 2.060 |
| KTINGSGSPHSKARMGQT | 1289 | 2.564 | KTINGSGSPHSKAGRGQT | 1646 | 2.058 |
| KTINGSGSPHSKAQVKLT | 1290 | 2.564 | KTINGSGSPHSKLMRQQT | 1647 | 2.056 |
| KTINGSGSPHSKLPRQQT | 1291 | 2.562 | KTINGSGSPHSKANKSQT | 1648 | 2.056 |
| KTINGSGSPHSKLCLQQT | 1292 | 2.562 | KTINGSGSPHSKAVRQQT | 1649 | 2.052 |
| KTINGSGSPHSPLWNQQT | 1293 | 2.562 | KTINGSGSPHSKCLSQQT | 1650 | 2.052 |
| KTINGSVSPHSWAQNQQT | 1294 | 2.562 | KTINGSGSPHSKAQWVLT | 1651 | 2.052 |
| KTIRSKGSPHSKAQNQQT | 1295 | 2.559 | KTINGSGSPHSKAQFWVT | 1652 | 2.050 |
| KTINGSRSPHSWAQNQQT | 1296 | 2.559 | KTINGSGSPHSKALCRQT | 1653 | 2.048 |
| KTINGSGSPHSKILRQQT | 1297 | 2.557 | KEVMGSGSPHSKAQNQQT | 1654 | 2.047 |
| KTINGRQSPHVKAQNQQT | 1298 | 2.557 | KTINGSGSPHSKNTRQQT | 1655 | 2.047 |
| KTINGSGSPHSKAQSIKT | 1299 | 2.555 | KTINGSGSPHTWTQNQQT | 1656 | 2.045 |
| KTINGSGSPHSKAQASKT | 1300 | 2.546 | KTINGSTSPHWSAQNQQT | 1657 | 2.043 |
| KTINGSGSPHSRLFNQQT | 1301 | 2.542 | KTINGNVSPHRKAQNQQT | 1658 | 2.043 |
| KTINGSGSPHIYLQNQQT | 1302 | 2.542 | KTINGSTSPHLFAQNQQT | 1659 | 2.041 |
| KTINGSGSPHSRVRNQQT | 1303 | 2.540 | KTINGSGSPHSKAQNYRA | 1660 | 2.039 |
| KTINGSGSPHSKAVRAQT | 1304 | 2.538 | KTINGSGSPHSKARGQQT | 1661 | 2.039 |
| KTINGSGSPHSKPARQQT | 1305 | 2.538 | KTINGSGSPHSKAQNRIR | 1662 | 2.039 |
| KTINGSGSPHSRYSNQQT | 1306 | 2.536 | KTINGSGSPHSKWTLQQT | 1663 | 2.039 |
| KTINGSRSPHRSAQNQQT | 1307 | 2.536 | KTINGSGSPHSKAQMKCT | 1664 | 2.039 |
| KTINGSLSPHIYAQNQQT | 1308 | 2.536 | KTINGSGSPHSLWQNQQT | 1665 | 2.037 |
| KTINGSGSPHSKPVRQQT | 1309 | 2.529 | KTINGSGSPHSKAQLSKT | 1666 | 2.035 |
| KTINGMRSPHGKAQNQQT | 1310 | 2.527 | KTINLIWSPHSKAQNQQT | 1667 | 2.035 |
| KTINGSGSPHSKARITQT | 1311 | 2.525 | KTINGSGSPHSKRVLQQT | 1668 | 2.035 |
| KTINGSGSPHSWSLNQQT | 1312 | 2.523 | KTINGSGSPHSKVRVQQT | 1669 | 2.034 |
| KTINTSRSPHSKAQNQQT | 1313 | 2.520 | KTINSRESPHSKAQNQQT | 1670 | 2.032 |
| KTINGSGSPHSKAFTRQT | 1314 | 2.518 | KRSKGSGSPHSKAQNQQT | 1671 | 2.030 |
| KTINGSGSPHSKAVRNQT | 1315 | 2.516 | KTINGSGSPHRRLQNQQT | 1672 | 2.030 |
| KTINGSGSPHSKAQTNRT | 1316 | 2.510 | KTINGSGSPHSCAQNQQT | 1673 | 2.030 |
| KTINGSGSPHSKANRMQT | 1317 | 2.508 | KTINGPLSPHRKAQNQQT | 1674 | 2.028 |
| KTINGSGSPHSKAQLVLT | 1318 | 2.508 | KTINGSVSPHLYAQNQQT | 1675 | 2.028 |
| KTINGSGSPHSKATRQQT | 1319 | 2.505 | KTINGRISPHLKAQNQQT | 1676 | 2.028 |
| KTINGSGSPHSKARGTQT | 1320 | 2.505 | KTINGSHSPHRKAQNQQT | 1677 | 2.028 |
| KTINGSGSPHSKAQWSVT | 1321 | 2.501 | KTINGSGSPHSKAQVSIT | 1678 | 2.028 |
| KTINGSGSPHSKAWLIQT | 1322 | 2.499 | KTINGSMSPHRRAQNQQT | 1679 | 2.028 |
| KTINGSGSPHSKAFRPQT | 1323 | 2.499 | KTINGRQSPHAKAQNQQT | 1680 | 2.026 |
| KTINGSGSPHRRSQNQQT | 1324 | 2.497 | KTINGSGSPHSKAVWRQT | 1681 | 2.026 |
| KTINGSGSPHSKGIRQQT | 1325 | 2.497 | KQPLGSGSPHSKAQNQQT | 1682 | 2.024 |
| KTINGSGSPHCTLQNQQT | 1326 | 2.497 | KTINGSGSPHSKAQNVKL | 1683 | 2.024 |
| KPLPGSGSPHSKAQNQQT | 1327 | 2.495 | KTINGSGSPHSKRGTQQT | 1684 | 2.022 |
| KTINGSGSPHLVCQNQQT | 1328 | 2.493 | KTINGSVSPHYVAQNQQT | 1685 | 2.022 |
| KTINGSGSPHSKARGYQT | 1329 | 2.492 | KTINGSGSPHSKNLRQQT | 1686 | 2.022 |
| KTINGRISPHGKAQNQQT | 1330 | 2.492 | KTINGSGSPHSKAQAFRT | 1687 | 2.022 |
| KTINGSSSPHWLAQNQQT | 133 | 2.490 | KTINGSGSPHSKCSNQQT | 1688 | 2.020 |
| KTINGSGSPHSKARMAQT | 1332 | 2.488 | KELVGSGSPHSKAQNQQT | 1689 | 2.019 |
| KPLDGSGSPHSKAQNQQT | 1333 | 2.488 | KTINGSGSPHSLVFNQQT | 1690 | 2.019 |
| KPLRGSGSPHSKAQNQQT | 1334 | 2.486 | KTINGSGSPHSKAQATRT | 1691 | 2.019 |
| KTINGSGSPHSKAQNAKL | 1335 | 2.486 | KTINGTSSPHCKAQNQQT | 1692 | 2.017 |
| KTINGSGSPHSKLSKQQT | 1336 | 2.482 | KTINGSGSPHSKALWRQT | 1693 | 2.015 |
| KTINGSGSPHSKARNGQT | 1337 | 2.482 | KTINGSGSPHSKAQFSVT | 1694 | 2.015 |
| KTINGSGSPHSKAQRRQT | 1338 | 2.479 | KTINGSGSPHSKLYMQQT | 1695 | 2.015 |
| KTINGSGSPHSKWPGQQT | 1339 | 2.477 | KTINGSLSPHYMAQNQQT | 1696 | 2.015 |
| KTINGSGSPHAFLQNQQT | 1340 | 2.475 | KTINGSGSPHSKAWLMQT | 1697 | 2.015 |
| KTINGILSPHRKAQNQQT | 1341 | 2.475 | KTINGSGSPHSKSLKQQT | 1698 | 2.013 |
| KTINGSGSPHSWGSNQQT | 1342 | 2.473 | KTINGSGSPHSKAQNTRR | 1699 | 2.013 |
| KTINGSGSPHSSCLNQQT | 1343 | 2.471 | KTINGSGSPHYLLQNQQT | 1700 | 2.011 |
| KTINGSGSPHSKAQSVKT | 1344 | 2.464 | KTINGSGSPHTWSQNQQT | 1701 | 2.011 |
| KTINGSGSPHSLRYNQQT | 1345 | 2.462 | KTINGSGSPHSKTRMQQT | 1702 | 2.011 |
| KTINGSGSPHSKARKLQT | 1346 | 2.458 | KTINTRPSPHSKAQNQQT | 1703 | 2.011 |
| KHRSGSGSPHSKAQNQQT | 1347 | 2.456 | KTINGSGSPHSKAQILVT | 1704 | 2.009 |
| KTINGSGSPHSKWSLQQT | 1348 | 2.453 | KTINGSGSPHSKAQNAKS | 1705 | 2.009 |
| KTINGSGSPHSKAQTMRT | 1349 | 2.453 | KTINGSGSPHSRTYNQQT | 1706 | 2.009 |
| KTINGSGSPHSKTIRQQT | 1350 | 2.453 | KTINGSGSPHSKKGGQQT | 1707 | 2.009 |
| KTINGKLSPHMKAQNQQT | 1351 | 2.449 | KTINGYSSPHRKAQNQQT | 1708 | 2.007 |
| KTINGSGSPHSKARPFQT | 1352 | 2.443 | KTINGSGSPHWVSQNQQT | 1709 | 2.007 |
| KTINGSGSPHSKPRVQQT | 1353 | 2.441 | KTINGSGSPHSKARLAQT | 1710 | 2.007 |
| KTINGSGSPHSKAQVVLT | 1354 | 2.438 | KTINGMCSPHSKAQNQQT | 1711 | 2.006 |
| KTRRSSGSPHSKAQNQQT | 1355 | 2.438 | KTINGSGSPHSKSNKQQT | 1712 | 2.006 |
| KTINGSGSPHSKPSRQQT | 1356 | 2.438 | KTINGSGSPHSKAQFVLT | 1713 | 2.006 |
| KTINGSGSPHSVYRNQQT | 1357 | 2.432 | KTINGSISPHFVAQNQQT | 1714 | 2.006 |
| KTINGSGSPHSKTCSQQT | 1358 | 2.430 | KTINGSGSPHRRMQNQQT | 1715 | 2.004 |
| KTINGSPSPHRKAQNQQT | 1359 | 2.430 | KTINGSGSPHSKAWILQT | 1716 | 2.004 |
| KTINGRSSPHFKAQNQQT | 1360 | 2.428 | KTINGSGSPHSKAQGVKT | 1717 | 2.002 |
| KTINGSGSPHSKAQMVRT | 1361 | 2.426 | KTINGSGSPHSKAQFSLT | 1718 | 2.000 |
| KTINGSRSPHCSAQNQQT | 1362 | 2.426 | KTINMLRSPHSKAQNQQT | 1719 | 2.000 |
| KTINGSCSPHLRAQNQQT | 1363 | 2.423 | KTINGSGSPHSKAQLGKT | 1720 | 2.000 |
| KTINGSGSPHSKAQCLFT | 1364 | 2.421 | KTINGSGSPHSMYLNQQT | 1721 | 2.000 |

As shown in Table 21, approximately 72 TTM-002 matured capsid variants demonstrated at least a 2-fold increase in expression relative to the non-matured TTM-002 control, with a few variants demonstrating greater than a three- to five-fold enrichment relative to the non-matured TTM-002 control. Also, across the peptides comprised within the TTM-002 matured capsid variants with the greatest fold-enrichment relative to the non-matured TTM-002 capsid in the brain, it was observed that the modifications in the variant sequences appeared in the region N-terminal to the SPH motif present within the capsid variant. This indicates that modifications that appeared to improve TTM-002 capsid tropism in the CNS of mice were skewed to the N-terminal portion of the peptide insertion in loop IV of the sequence. Additionally, a number of these N-terminal modifications that were incorporated into the matured TTM-002 capsid variants were negatively charged amino acids (particularly glutamic acid (E)).

TABLE 21
NGS fold-enrichment of TTM-002 matured AAV capsid variants in the brain of CD-1
Outbred mice

| | | Fold | | | Fold |
|---|---|---|---|---|---|
| | SEQ | enrichment | | SEQ | enrichment |
| | ID | over TTM- | | ID | over TTM- |
| Peptide Sequence | NO: | 002 | Peptide Sequence | NO: | 002 |
| KTINGHDSPHVTDQNQQT | 1722 | 5.20 | KAEVGHDSPHKSGQNQQT | 1760 | 2.15 |
| KTINGHDSPHKRGQHRQT | 1723 | 4.20 | KMDAGHDSPHKSGQNQQT | 1761 | 2.15 |
| KMPEGHDSPHKSGQNQQT | 1724 | 3.18 | KVEWGHDSPHKSGQNQQT | 1762 | 2.15 |
| KMEGGHDSPHKSGQNQQT | 1725 | 2.72 | KAEQGHDSPHKSGQNQQT | 1763 | 2.14 |
| KMEYGHDSPHKSGQNQQT | 1726 | 2.71 | KLEWGHDSPHKSGQNQQT | 1764 | 2.14 |
| KAEWGHDSPHKSGQNQQT | 1727 | 2.69 | KTINGHPSPHYLGQNQQT | 1765 | 2.14 |
| KCEWGHDSPHKSGQNQQT | 1728 | 2.68 | KTINGHLSPHYYGQNQQT | 1766 | 2.13 |
| KANNGQDSPHKSGQNQQT | 1729 | 2.67 | KMELGHDSPHKSGQNQQT | 1767 | 2.13 |
| KTINGHDSPHLCGQNQQT | 1730 | 2.59 | KMETGHDSPHKSGQNQQT | 1768 | 2.12 |
| KIPEGHDSPHKSGQNQQT | 1731 | 2.54 | KMEAGHDSPHKSGQNQQT | 1769 | 2.12 |
| KADMGHDSPHKSGQNQQT | 1732 | 2.48 | KTINGHDSPHLLWQNQQT | 1770 | 2.12 |
| KTINGHLSPHYFGQNQQT | 1733 | 2.41 | KTINRQRSPHKSGQNQQT | 1771 | 2.11 |
| KIEYGHDSPHKSGQNQQT | 1734 | 2.41 | KIESGHDSPHKSGQNQQT | 1772 | 2.11 |
| KADYGHDSPHKSGQNQQT | 1735 | 2.40 | KTAKDHDSPHKSGQNQQT | 1773 | 2.11 |
| KIETGHDSPHKSGQNQQT | 1736 | 2.38 | KMEVGHDSPHKSGQNQQT | 1774 | 2.11 |
| KTINGHDSPHINGQKQQT | 1737 | 2.38 | KCEIGHDSPHKSGQNQQT | 1775 | 2.10 |
| KMEWGHDSPHKSGQNQQT | 1738 | 2.38 | KATNGHDSPHKSGLNQQT | 1776 | 2.10 |
| KTINGHDSPHWLLQNQQT | 1739 | 2.37 | KMDGGHDSPHKSGQNQQT | 1777 | 2.09 |
| KCEYGHDSPHKSGQNQQT | 1740 | 2.36 | KQEVGHDSPHKSGQNQQT | 1778 | 2.07 |
| KRINGHDSPHKSGQKQQN | 1741 | 2.34 | KADQGHDSPHKSGQNQQT | 1779 | 2.07 |
| KMEIGHDSPHKSGQNQQT | 1742 | 2.34 | KTINGHESPHKSAQNHQT | 1780 | 2.07 |
| KLEYGHDSPHKSGQNQQT | 1743 | 2.33 | KTINGHDSPHKSAQNQWT | 1781 | 2.07 |
| KADWGHDSPHKSGQNQQT | 1744 | 2.32 | KNMNGHDSPHKSGQNTHS | 1782 | 2.06 |
| KIEIGHDSPHKSGQNQQT | 1745 | 2.30 | KTPWEHDSPHKSGQNQQT | 1783 | 2.05 |
| KTIKDNDSPHKSGQNQQT | 1746 | 2.27 | KTINGHSSPHYFGQNQQT | 1784 | 2.05 |
| KDIMGHDSPHKSGQNQQT | 1747 | 2.23 | KIEMGHDSPHKSGQNQQT | 1785 | 2.05 |
| KFEQGHDSPHKSGQNQQT | 1748 | 2.22 | KTANEHDSPHKSGQNQQT | 1786 | 2.05 |
| KMEFGHDSPHKSGQNQQT | 1749 | 2.21 | KTINGHDSPHKSGRRRQT | 1787 | 2.04 |
| KCDQGHDSPHKSGQNQQT | 1750 | 2.21 | KISNGHDSPHKSAQNQQT | 1788 | 2.03 |
| KLPEGHDSPHKSGQNQQT | 1751 | 2.19 | KTGNGHDSPHKSGQYQQT | 1789 | 2.03 |
| KIENGHDSPHKSGQNQQT | 1752 | 2.19 | KTINGHYSPHLFGQNQQT | 1790 | 2.02 |
| KMESGHDSPHKSGQNQQT | 1753 | 2.18 | KTINGNYSPHKIGQNQQT | 1791 | 2.02 |
| KAEIGHDSPHKSGQNQQT | 1754 | 2.17 | KTINGHDSPHKSRQNDQT | 1792 | 2.01 |
| KVEYGHDSPHKSGQNQQT | 1755 | 2.17 | KQQQGHDSPHKSGQNQQT | 1793 | 2.01 |
| KIINGHDSPHKSGLTQQT | 1756 | 2.17 | KTPQDHDSPHKSGQNQQT | 1794 | 2.00 |
| KTSNGDDSPHKSGRNQQT | 1757 | 2.17 | KHDWGHDSPHKSGQNQQT | 1795 | 2.00 |
| KIEVGHDSPHKSGQNQQT | 1758 | 2.16 | KIEGGHDSPHKSGQNQQT | 1796 | 2.00 |
| KMEMGHDSPHKSGQNQQT | 1759 | 2.16 | | | |

These data demonstrate that following two maturation approaches, matured TTM-001 and TTM-002 capsid variants with loop IV modifications were generated with significantly enhanced CNS tropism in mice compared to the corresponding non-matured TTM-001 and TTM-002 capsid variants, which already exhibited a significant fold enrichment over AAV9 in the mouse brain.

### Example 4. Maturation of TTM-001 and TTM-002 Capsid in NHPs

This Example describes maturation of the AAV9 capsid variants, TM-001 (SEQ ID NO: 981 (amino acid) and 983 (DNA), comprising SEQ ID NO: 941 (encoded by SEQ ID NO: 942)) and TTM-002 (SEQ ID NO: 982 (amino acid) and 984 (DNA), comprising SEQ ID NO: 2 (encoded by SEQ ID NO: 3)) in NHPs to further enhance their transduction and biodistribution in the central nervous system as well as other tissues, and evolve the AAV capsid variants to provide further cross-species compatibility. Two approaches were used to mature the TTM-001 and TM-002 capsid sequences in order to randomize and mutate within and around the peptide insert comprised within loop TV of the capsid variant. As many of the AAV capsid variants that demonstrated the greatest fold-enrichment in the NHP brain relative wild-type AAV9 comprised an SPH motif in the same position (e.g., immediately subsequent to position 455, relative to a reference sequence numbered according to the amino acid sequence of SEQ ID NO: 138) (see Example 1), the SPH motif was not mutated in either approach to mature the TTM-001 and TTM-002 capsid variants. In the first maturation approach, sets of three contiguous amino acids were randomized across the mutagenesis region in the TTM-00 I and TTM-002 sequences, which spanned from position 450 to position 466, numbered according to SEQ ID NO: 981 and 982. In the second maturation approach, mutagenic primers were used to introduce point mutations at a low frequency, scattered across the mutagenesis region in the TTM-001 and TTM-002 sequences ranging from position 449 to position 466, numbered according to SEQ ID NO: 981 and 982. AAV capsid variants arising from each maturation approach for TTM-00I and TTM-002 were pooled together, for subsequent testing and characterization in NHPs.

The library of pooled matured AAV capsid variants generated using the first maturation approach and the second maturation approach for the TTM-00I and TTM-02 AAV capsid variants were injected into two NHPs. After a period in life, the brains, heart, liver, muscle, and DRG of the NHPs were isolated and RNA was extracted. Following RNA recovery and RT-PCR amplification, a systematic NGS enrichment analysis was performed to calculate the fold enrichment ratio relative to an AAV9 control, and the peptides comprised within the variants were identified.

Following the RNA recovery and NGS analysis from the second maturation approach, approximately 680,000 capsid variants were identified. The 680,000 matured capsid variants were then filtered based on samples with a raw virus count greater than 10 and a coefficient of variance (CV) of less than 1, which was calculated for each peptide across the brain samples taken from the two NHPs. Those that had a CV value <1 were identified, as these were the peptides that were reliably detected in the majority of samples isolated from the brains of the two NHPs. Using this filtering criteria, this led to approximately 64,000 matured capsid variants.

Table 22 provides the peptide sequences of the matured capsid variants having a raw virus count greater than 10, a CV of less than 1 for the brain samples isolated, and that also demonstrated a 50-fold or greater fold-increase in expression in the brain relative to the AAV9 control in both mice and NHPs. The matured variants in Table 22, were also those variants that had a fold-change in expression that was less than 2 relative to the AAV9 control in the liver and the DRG. Applying these criteria, approximately 350 matured capsid variants were identified that demonstrated high transduction in the brain in NHPs and mice, cross-species compatibility in mice and NHPs, and were de-targeted in the liver and DRG, relative to the AAV9 control. Several variants as shown in Table 22, led to greater than 100-fold increase in expression relative to AAV9 in the NHP and/or mouse brain, with one variant resulting in a greater than 200-fold increase in expression relative to AAV9 in both species.

Fold-change in expression for the TTM-001 and TTM-002 matured variants in Table 22 that showed increased expression in the brain of the NHPs and mice, were also calculated for the DRG, muscle, liver (RNA and DNA), and heart of the NHPs following each maturation approach. As shown in Table 22, many variants were de-targeted in the peripheral tissues with a lower fold-change in expression relative to the AAV9 control, demonstrating CNS-specific tropism and a preferential transduction of the brain and CNS. Some variants demonstrated increased expression to AAV9 in multiple tissues, including the brain and peripheral tissues, demonstrating pan-tropism.

TABLE 22
NGS fold-enrichment of TTM-001 and TTM-002 matured AAV capsid variants in the
brain of NHPs and mice

| | | Fold Enrichment relative to AAV9 | | | | | | |
|---|---|---|---|---|---|---|---|---|
| | SEQ | | | | | Liver | Liver | |
| | ID | Brain | DRG | Heart | Muscle | RNA | DNA | Brain |
| Sequence | NO: | (NHP) | (NHP) | (NHP) | (NHP) | (NHP) | NHP | (Mouse) |
| KTQQGSGSPHSKAQNRHT | 3239 | 217.176 | 0.000 | 0.000 | 0.000 | 0.000 | 0.000 | 210.515 |
| KTFPGSGSPHSKVQNQQT | 3240 | 199.720 | 0.000 | 0.000 | 0.000 | 0.000 | 0.967 | 97.703 |
| KTEKMSGSPHSKAQNQQT | 3241 | 169.461 | 0.523 | 0.000 | 0.000 | 0.000 | 0.158 | 109.161 |
| KEQNGRGSPHSKAQNQQT | 3527 | 134.390 | 0.239 | 0.000 | 0.000 | 0.000 | 0.232 | 52.311 |
| KTVNRNGSPHSKAQNQQT | 3528 | 133.016 | 0.000 | 0.416 | 0.000 | 0.000 | 0.000 | 85.361 |
| KTVNGSGSPHSKARDQQT | 3242 | 124.789 | 0.123 | 0.039 | 0.312 | 0.569 | 0.454 | 132.137 |
| KTENGSGSPHSKAPNLQT | 3243 | 121.436 | 0.000 | 0.167 | 0.000 | 0.000 | 0.015 | 168.920 |
| KTEKTSGSPHSKAQNQQT | 3244 | 120.337 | 0.000 | 0.355 | 0.000 | 0.000 | 0.119 | 101.467 |
| KTQNGSGSPHSKAHVRQT | 3245 | 119.798 | 0.000 | 0.000 | 0.262 | 0.694 | 1.039 | 165.590 |
| KTVNGSGSPHSKAPNQHT | 3246 | 117.207 | 0.000 | 0.109 | 0.000 | 0.000 | 0.074 | 51.008 |
| KTEKQSGSPHSKAQNQQT | 3247 | 116.603 | 0.000 | 0.000 | 0.000 | 0.000 | 0.426 | 102.978 |
| KTQNGPGSPHSKAHNQQT | 3529 | 115.742 | 0.146 | 0.000 | 0.235 | 0.000 | 0.513 | 52.508 |
| KTVNGSGSPHSKTQSQQT | 3248 | 115.086 | 0.000 | 0.726 | 0.000 | 0.000 | 0.340 | 63.248 |
| TTQNGSGSPHSKAQNQQT | 3249 | 114.856 | 1.340 | 14.856 | 0.827 | 1.281 | 0.957 | 72.058 |
| KSQNESGSPHSKAQNQQQ | 3250 | 113.833 | 0.000 | 0.000 | 0.000 | 0.000 | 0.000 | 67.649 |
| KTERTSGSPHSKAQNQQT | 3251 | 112.957 | 0.000 | 0.009 | 0.000 | 1.128 | 0.20 | 117.374 |
| KTQNGSGSPHSKAQPAKT | 3252 | 111.472 | 0.331 | 0.000 | 1.089 | 0.044 | 1.796 | 215.275 |
| KTEKSSGSPHSKAQNQQT | 3253 | 107.470 | 0.000 | 0.016 | 0.014 | 0.97 | 0.179 | 100.177 |
| KTSYGNGSPHSKAQNQQT | 3530 | 105.937 | 0.000 | 0.000 | 0.000 | 0.000 | 0.114 | 105.894 |
| KTEKGSGSPHSKAQNQQT | 3254 | 105.614 | 0.053 | 0.031 | 0.000 | 0.586 | 0.169 | 84.653 |
| KTQNGSGSPHSKSQTQQN | 3255 | 104.474 | 0.000 | 0.131 | 0.000 | 0.084 | 0.038 | 54.021 |
| KTERQSGSPHSKAQNQQT | 3256 | 103.692 | 0.000 | 0.000 | 0.000 | 0.062 | 0.370 | 89.637 |
| KTERASGSPHSKAQNQQT | 3257 | 103.669 | 0.000 | 0.000 | 0.000 | 0.127 | 0.070 | 115.550 |
| KELHGSGSPHSKAQNQQT | 3258 | 102.680 | 0.000 | 0.000 | 0.000 | 1.634 | 0.592 | 96.554 |
| KAQNGSGSPHSKAQNLAT | 3259 | 101.954 | 0.000 | 10.954 | 8.655 | 0.298 | 0.239 | 116.685 |
| KTVNGSGSPHSKSQNQLT | 3260 | 101.327 | 0.000 | 0.035 | 0.000 | 0.000 | 0.025 | 80.716 |
| KTERNSGSPHSKAQNQQT | 3261 | 99.892 | 0.000 | 0.000 | 0.000 | 0.000 | 0.107 | 87.392 |
| KSVNGNGSPHSKAQNQQT | 3531 | 99.385 | 0.000 | 1.329 | 0.000 | 0.359 | 0.079 | 51.016 |
| KTENGSGSPHSKAQGQQT | 3262 | 99.253 | 0.000 | 0.208 | 0.000 | 0.128 | 0.099 | 81.459 |
| KTQNGSGSPHGWVQNQQT | 3532 | 97.122 | 0.000 | 0.000 | 0.000 | 1.240 | 1.975 | 290.720 |
| KTERVSGSPHSKAQNQQT | 3263 | 96.943 | 0.000 | 0.000 | 0.000 | 0.000 | 0.144 | 135.438 |
| KTQNGSGSPHSKALNRQS | 3264 | 96.843 | 0.136 | 0.532 | 0.000 | 0.042 | 0.178 | 55.945 |
| KTERLSGSPHSKAQNQQT | 3265 | 95.857 | 0.000 | 0.004 | 0.005 | 0.126 | 0.260 | 102.372 |
| KTDNGSGSPHSKAHNQQT | 3266 | 95.164 | 0.000 | 0.000 | 0.000 | 0.000 | 0.027 | 55.313 |
| KTFHGSGSPHSKTQNQQT | 3267 | 94.714 | 0.000 | 0.210 | 0.120 | 0.000 | 0.000 | 51.119 |
| KTQNGGGSPHSKAQTQQQ | 3533 | 92.345 | 0.000 | 0.000 | 0.000 | 0.000 | 0.023 | 54.199 |
| KTSNGSGSPHSKAQNPPT | 3268 | 91.528 | 0.000 | 0.000 | 0.000 | 0.000 | 0.039 | 51.541 |
| ETQNGSGSPHSKAQNLQT | 3269 | 90.969 | 0.221 | 1.023 | 0.197 | 0.179 | 0.813 | 107.216 |
| KTVHGNGSPHSKAQNQQT | 3534 | 90.073 | 0.000 | 0.000 | 0.000 | 0.000 | 0.304 | 97.003 |
| NTQNGSGSPHSKAQNQQT | 3270 | 90.017 | 1.712 | 1.261 | 1.171 | 0.923 | 0.540 | 55.179 |
| KTQNGGGSPHSKAQNQQC | 3535 | 89.301 | 0.219 | 0.000 | 0.000 | 0.287 | 0.319 | 53.840 |
| KTENMSGSPHSKAQNQQT | 3271 | 89.247 | 0.000 | 0.000 | 0.000 | 0.000 | 0.260 | 130.568 |
| KTENVSGSPHSKAQNQQT | 3272 | 88.506 | 0.000 | 0.000 | 0.000 | 0.964 | 0.112 | 108.591 |
| KTSSGSGSPHSKAQYQQT | 3273 | 87.304 | 0.000 | 0.000 | 0.000 | 0.000 | 0.299 | 58.143 |
| KTQDGGGSPHSKAQNKQT | 3536 | 85.019 | 0.000 | 0.000 | 0.000 | 0.000 | 0.477 | 55.517 |
| KTEKVSGSPHSKAQNQQT | 3274 | 84.558 | 0.000 | 0.022 | 0.000 | 0.873 | 0.424 | 112.185 |
| KAQNGSGSPHSKAQDQET | 3275 | 84.080 | 0.000 | 0.000 | 0.000 | 0.194 | 0.027 | 87.637 |
| KTCNKSGSPHSKAQNQQT | 3276 | 83.992 | 0.000 | 0.000 | 0.165 | 0.283 | 0.000 | 119.496 |
| KTQNGGGSPHSKAQNQLQ | 3537 | 83.881 | 0.000 | 0.000 | 0.000 | 0.046 | 0.387 | 78.383 |
| KNQNGGGSPHSKAQNQQT | 3538 | 83.083 | 0.000 | 0.042 | 0.000 | 0.000 | 0.000 | 75.913 |
| KTEHLSGSPHSKAQNQQT | 3277 | 83.080 | 0.000 | 0.000 | 0.012 | 0.021 | 0.189 | 69.494 |
| KAEMGSGSPHSKAQNQQT | 3278 | 83.049 | 0.000 | 0.020 | 0.000 | 0.768 | 0.112 | 135.019 |
| KAQNGSGSPHSKAQNHQT | 3279 | 82.627 | 0.000 | 0.176 | 0.000 | 0.155 | 0.057 | 66.207 |
| KAQKGSGSPHSKAQDQQT | 3280 | 82.258 | 0.000 | 0.000 | 0.000 | 0.108 | 0.000 | 85.178 |
| KTQNGGGSPHSKSQNQLT | 3539 | 82.231 | 0.000 | 0.070 | 0.000 | 0.000 | 0.498 | 126.986 |
| KTVNGNGSPHSKAQNKQT | 3540 | 81.481 | 0.000 | 0.000 | 0.000 | 0.000 | 0.122 | 69.455 |
| KTQNGSGSPHSKGHWQQT | 3281 | 81.434 | 0.000 | 0.000 | 0.000 | 0.000 | 1.011 | 65.252 |
| KTDKTSGSPHSKAQNQQT | 3282 | 81.430 | 0.000 | 0.000 | 0.000 | 1.362 | 0.291 | 169.515 |
| KTFKGSGSPHSKAPNQQT | 3283 | 80.890 | 0.000 | 0.000 | 0.000 | 0.000 | 0.017 | 71.144 |
| KTVNGSGSPHSKAQNQLQ | 3284 | 80.509 | 0.000 | 0.000 | 0.000 | 0.000 | 0.166 | 71.156 |
| KTQNGSGSPHSKRPEQQT | 3285 | 80.418 | 0.000 | 0.013 | 0.000 | 0.149 | 0.361 | 50.319 |
| KTQNGSGSPHSKAQRTMT | 3286 | 80.388 | 0.000 | 0.022 | 0.170 | 1.812 | 1.025 | 00.248 |
| KTEKASGSPHSKAQNQQT | 3287 | 80.285 | 0.000 | 0.041 | 0.000 | 0.000 | 0.261 | 90.390 |
| KSDQGSGSPHSKAQNQQT | 3288 | 80.076 | 0.000 | 0.000 | 0.000 | 0.993 | 0.124 | 151.911 |
| KTEQTSGSPHSKAQNQQT | 3289 | 79.620 | 0.000 | 0.163 | 0.000 | 0.332 | 0.074 | 76.686 |
| KTDKSSGSPHSKAQNQQT | 3290 | 79.470 | 0.055 | 0.012 | 0.000 | 1.437 | 0.367 | 141.351 |
| KTQDGSGSPHSKAQNQQH | 3291 | 79.090 | 0.000 | 0.000 | 0.000 | 0.136 | 0.049 | 57.914 |
| KTVNGNGSPHSKAQNQHT | 3541 | 78.849 | 0.000 | 0.000 | 0.000 | 0.000 | 0.045 | 54.086 |
| KNQNGSGSPHSKAQNQQT | 3292 | 78.445 | 0.000 | 0.000 | 0.000 | 0.571 | 0.177 | 89.719 |
| KTETHSGSPHSKAQNQQT | 3293 | 77.974 | 0.000 | 0.067 | 0.000 | 0.000 | 0.512 | 57.287 |
| KTQNGGGSPHSKALNQQN | 3542 | 77.822 | 0.000 | 0.131 | 0.000 | 0.000 | 0.274 | 69.884 |
| KTQNGSGSPHSKALHQHT | 3294 | 77.502 | 0.000 | 0.052 | 0.041 | 0.000 | 0.188 | 68.196 |
| KTQNGTGSPHSKAQNHQQ | 3543 | 77.089 | 0.171 | 0.000 | 0.000 | 0.000 | 0.166 | 54.281 |
| KTQNGSGSPHSKAQHRQT | 3295 | 76.849 | 0.105 | 0.499 | 0.170 | 1.424 | 0.214 | 127.000 |
| KTQNGSGSPHSKAQYQHT | 3296 | 76.170 | 0.000 | 0.014 | 0.033 | 1.523 | 0.168 | 59.649 |
| KTENQSGSPHSKAQNQQT | 3297 | 76.072 | 0.000 | 0.000 | 0.000 | 0.115 | 0.132 | 83.118 |
| KTQQGGGSPHSKAHNQQT | 3544 | 75.872 | 0.000 | 0.050 | 0.000 | 0.000 | 0.235 | 65.492 |
| KTQNGSGSPHSKAQKFET | 3298 | 75.788 | 0.000 | 0.000 | 0.028 | 0.108 | 0.093 | 65.588 |
| KTSNESGSPHSKAQNHQT | 3299 | 75.720 | 0.000 | 0.000 | 0.000 | 0.169 | 0.217 | 70.590 |
| KTQNGSGSPHSKAQFPST | 3300 | 75.677 | 0.000 | 0.004 | 0.000 | 0.849 | 0.127 | 119.712 |
| KTERPSGSPHSKAQNQQT | 3301 | 75.669 | 0.000 | 0.029 | 0.000 | 0.000 | 0.156 | 73.894 |
| KTQNGNGSPHSKAQNPLT | 3545 | 75.269 | 0.000 | 0.000 | 0.000 | 0.366 | 0.000 | 53.583 |
| KSQKGNGSPHSKAQNQQT | 3546 | 75.196 | 0.000 | 0.000 | 0.000 | 0.000 | 0.000 | 90.251 |
| KTERMSGSPHSKAQNQQT | 3302 | 74.910 | 0.000 | 0.000 | 0.000 | 0.100 | 0.151 | 122.812 |
| KTERSSGSPHSKAQNQQT | 3303 | 74.853 | 0.000 | 0.071 | 0.000 | 1.036 | 0.056 | 125.538 |
| KTELHSGSPHSKAQNQQT | 3304 | 74.620 | 0.000 | 0.000 | 0.000 | 0.021 | 0.089 | 53.124 |
| KTELTSGSPHSKAQNQQT | 3305 | 74.548 | 0.000 | 0.000 | 0.000 | 0.537 | 0.421 | 100.311 |
| KTQNGSGSPHSKAHNQQR | 3306 | 74.272 | 0.562 | 0.486 | 0.047 | 0.956 | 0.057 | 107.301 |
| KTQNGGGSPHSKAQSQQQ | 3547 | 74.264 | 0.000 | 0.000 | 0.000 | 0.000 | 0.235 | 67.651 |
| KTQNGSGSPHSKAQAQKT | 3307 | 74.261 | 0.255 | 0.000 | 0.000 | 0.186 | 0.132 | 73.560 |
| KTENTSGSPHSKAQNQQT | 3308 | 74.061 | 0.000 | 0.000 | 0.218 | 0.233 | 0.730 | 96.249 |
| KTQDGSGSPHSKGQNRQT | 3309 | 73.930 | 0.000 | 0.000 | 0.000 | 0.106 | 0.091 | 63.626 |
| KNQNGSGSPHSKAQSQQT | 3310 | 73.757 | 0.000 | 0.000 | 0.000 | 0.000 | 0.041 | 57.432 |
| KTQNGSVSPHGKAQNQLT | 3548 | 73.525 | 0.000 | 0.061 | 0.067 | 0.000 | 0.053 | 51.358 |
| KTSNASGSPHSKAQNQLT | 3311 | 73.501 | 0.000 | 0.000 | 0.297 | 0.000 | 0.313 | 150.401 |
| KTEARSGSPHSKAQNQQT | 3312 | 73.349 | 0.000 | 0.000 | 0.000 | 0.695 | 0.118 | 62.903 |
| KTEKNSGSPHSKAQNQQT | 3313 | 73.347 | 0.000 | 0.000 | 0.044 | 0.159 | 0.021 | 74.393 |
| KTANGSGSPHSKAQYQQT | 3314 | 73.038 | 0.000 | 0.000 | 0.000 | 0.153 | 0.160 | 139.451 |
| KTVNGSGSPHSKAQYQHT | 3315 | 72.84″ | 0.000 | 0.000 | 0.000 | 0.000 | 0.130 | 54.158 |
| KTQNGSGSPHTKAQNPQS | 3316 | 72.594 | 0.000 | 0.000 | 0.000 | 0.000 | 0.130 | 62.508 |
| KTQNGSGSPHSKGQNPPT | 3317 | 72.339 | 0.000 | 0.206 | 0.000 | 0.000 | 0.04 | 134.808 |
| KTQQGSGSPHSKAQHQLT | 3318 | 72.291 | 0.000 | 0.000 | 0.000 | 0.000 | 0.000 | 100.144 |
| KTQNGSGSPHSKAQSPPT | 3319 | 71.632 | 0.069 | 0.047 | 0.274 | 0.179 | 0.425 | 97.111 |
| NTQYGSGSPHSKAQNQQT | 3320 | 71.267 | 1.739 | 0.000 | 273.69 | 0.000 | 0.209 | 59.707 |
| KTQNGSGSPHSKAQAKLT | 3321 | 71.154 | 0.000 | 0.273 | 0.017 | 1.591 | 0.777 | 130.132 |
| KTDKNSGSPHSKAQNQQT | 3322 | 70.964 | 0.000 | 0.000 | 0.000 | 0.070 | 0.123 | 62.932 |
| KTQNGSGSPHSKTKSQQT | 3323 | 70.891 | 0.000 | 0.568 | 0.045 | 0.418 | 0.496 | 83.923 |
| KTQNGSGSPHSKAQDRPT | 3324 | 70.831 | 0.132 | 0.006 | 0.000 | 0.039 | 0.379 | 66.800 |
| KTQNGQGSPHSKAQNLGT | 3549 | 70.543 | 0.000 | 0.071 | 0.000 | 0.000 | 0.135 | 104.769 |
| KTQNGSGSPHSKAQSQQL | 3325 | 70.539 | 0.000 | 0.000 | 0.000 | 0.000 | 0.041 | 51.126 |
| KTENLSGSPHSKAQNQQT | 3326 | 70.303 | 0.070 | 0.000 | 0.000 | 0.395 | 0.470 | 107.385 |
| KTQNGSGSPHSKAQAFHT | 3327 | 70.159 | 0.033 | 0.000 | 0.058 | 0.762 | 0.119 | 86.268 |
| KTQNGSGSPHSKAQKQQD | 3328 | 70.116 | 0.000 | 0.024 | 0.000 | 0.064 | 0.083 | 110.196 |
| KTFSGSGSPHSKAQNLQT | 3329 | 70.035 | 0.000 | 0.327 | 0.303 | 0.000 | 0.228 | 70.917 |
| KAQNGSGSPHSKAQNAQT | 3330 | 69.651 | 0.000 | 0.000 | 0.000 | 0.023 | 0.142 | 72.160 |
| KTESWSGSPHSKAQNQQT | 3331 | 69.144 | 0.000 | 0.000 | 0.000 | 0.000 | 0.019 | 67.699 |
| KTQNGSGSPHSKAHNQLT | 3332 | 69.062 | 0.000 | 0.000 | 0.000 | 0.708 | 0.000 | 65.505 |
| KTVNGNGSPHSKAQNHQT | 3550 | 68.889 | 0.000 | 0.000 | 0.000 | 0.000 | 0.030 | 52.482 |
| KTEDKSGSPHSKAQNQQT | 3333 | 68.813 | 0.000 | 0.000 | 0.000 | 0.000 | 0.000 | 70.071 |
| KTESASGSPHSKAQNQQT | 3334 | 68.651 | 0.000 | 0.000 | 0.000 | 0.274 | 0.084 | 80.500 |
| KTNNGSGSPHSKAQNQQY | 3335 | 68.530 | 0.000 | 0.040 | 0.000 | 0.000 | 0.059 | 82.656 |
| KTSNGGGSPHSKAQNLQT | 3551 | 68.311 | 0.000 | 0.052 | 0.000 | 0.000 | 0.000 | 124.87 |
| KTDKMSGSPHSKAQNQQT | 3336 | 68.167 | 0.000 | 0.000 | 0.000 | 0.017 | 0.205 | 88.234 |
| KEVHGSGSPHSKAQNQQT | 3337 | 67.901 | 0.000 | 0.000 | 0.000 | 0.727 | 0.000 | 100.111 |
| KTQNGSGSPHSKAQKLNT | 3338 | 67.782 | 0.073 | 0.092 | 0.000 | 1.232 | 0.201 | 68.637 |
| KTQNGGGSPHSKSQNQHT | 3552 | 67.773 | 0.000 | 0.057 | 0.000 | 0.000 | 0.220 | 100.748 |
| KTVNGGGSPHSKAQSQQT | 3553 | 67.634 | 0.000 | 0.055 | 0.000 | 0.000 | 0.210 | 160.711 |
| KTTNGSGSPHSKAQYQHT | 3339 | 67.325 | 0.000 | 0.000 | 0.000 | 1.378 | 0.080 | 83.337 |
| KTQSGSGSPHSKAQYQHT | 3340 | 66.739 | 0.000 | 0.000 | 0.000 | 0.000 | 0.191 | 59.822 |
| KTESTSGSPHSKAQNQQT | 3341 | 66.649 | 0.000 | 0.009 | 0.000 | 1.688 | 0.176 | 95.861 |
| KTQNGSGSPHSKSQNVQT | 3342 | 66.627 | 0.000 | 0.190 | 0.000 | 0.202 | 0.188 | 56.672 |
| KSQNGSGSPHSKAQAQQT | 3343 | 66.464 | 0.000 | 0.711 | 0.000 | 0.148 | 0.111 | 78.451 |
| KTVNGSGSPHSKAQNLQA | 3344 | 66.379 | 0.000 | 0.000 | 0.000 | 0.000 | 0.132 | 50.934 |
| KTVRDSGSPHSKAQNQQT | 3345 | 66.056 | 0.000 | 0.025 | 0.000 | 0.129 | 0.461 | 142.600 |
| KTFNASGSPHSKAPNQQT | 3346 | 65.392 | 0.208 | 0.000 | 0.000 | 0.215 | 0.156 | 66.275 |
| KTDRMSGSPHSKAQNQQT | 3347 | 65.143 | 0.000 | 0.000 | 0.000 | 0.332 | 0.103 | 104.890 |
| KTQNGSGSPHSKAQTPPT | 3348 | 64.657 | 0.010 | 0.015 | 0.014 | 0.200 | 0.207 | 54.179 |
| ETQKGSGSPHSKAQNQQT | 3349 | 64.609 | 0.000 | 0.000 | 0.144 | 0.000 | 0.024 | 67.20 |
| KNHQGSGSPHSKAQNQQT | 3350 | 64.535 | 0.000 | 0.000 | 0.000 | 1.253 | 0.187 | 70.356 |
| KTQNGSGSPHSKAQYQHA | 3351 | 64.435 | 0.000 | 0.000 | 0.024 | 0.993 | 0.097 | 57.278 |
| KTQPQDGSPHSKAQNQQT | 3554 | 64.421 | 0.000 | 0.047 | 0.000 | 0.234 | 0.936 | 76.826 |
| KTQNGSGSPHSKAQGQQA | 3352 | 64.128 | 0.000 | 0.185 | 0.000 | 0.063 | 0.195 | 64.116 |
| KTENGSGSPHNKAQNHQT | 3353 | 64.060 | 0.000 | 0.000 | 0.035 | 0.094 | 0.317 | 67.757 |
| KESDGSGSPHSKAQNQQT | 3354 | 63.766 | 0.000 | 0.000 | 0.000 | 0.567 | 0.146 | 115.231 |
| KTQNGSGSPHSKAQPPAT | 3355 | 63.510 | 0.048 | 0.030 | 0.031 | 0.126 | 0.302 | 117.453 |
| KTQNGSGSPHSKAQERPT | 3356 | 63.460 | 0.000 | 0.01 | 0.000 | 0.810 | 0.173 | 57.506 |
| KTQKGSGSPHSKAQDLQT | 3357 | 63.260 | 0.000 | 0.000 | 0.000 | 0.000 | 0.218 | 58.576 |
| KTDLKSGSPHSKAQNQQT | 3358 | 63.152 | 0.000 | 0.000 | 0.012 | 0.285 | 0.377 | 62.687 |
| KTQNGGGSPHSKAQNPPT | 3555 | 63.041 | 0.000 | 0.082 | 0.000 | 0.000 | 0.057 | 64.045 |
| KTQNGSGSPHSKAQAMHT | 3359 | 62.756 | 0.000 | 0.000 | 0.010 | 0.976 | 0.393 | 84.056 |
| KTVPNSGSPHSKAQNQQT | 3360 | 62.540 | 0.000 | 0.000 | 0.011 | 0.202 | 0.161 | 93.793 |
| KTVQGSGSPHSKALNQQT | 3361 | 62.358 | 0.000 | 0.310 | 0.000 | 0.062 | 0.245 | 60.369 |
| KTQNGSGSPHSKAQHPST | 3362 | 62.255 | 0.000 | 0.044 | 0.000 | 1.345 | 0.301 | 101.103 |
| KTQNGLGSPHSKSQNQQT | 3556 | 62.170 | 0.000 | 0.157 | 0.000 | 0.146 | 0.107 | 64.139 |
| KTQNGTGSPHSKAQNQQM | 3557 | 62.151 | 0.000 | 0.000 | 0.000 | 0.000 | 0.000 | 62.376 |
| KTQNGSGSPHSKAPGLQT | 3363 | 62.043 | 0.007 | 0.000 | 0.005 | 0.651 | 0.210 | 144.610 |
| KTQNGSGSPHSKAQGQRT | 3364 | 61.952 | 0.041 | 0.000 | 0.012 | 0.897 | 0.502 | 155.013 |
| KTESHSGSPHSKAQNQQT | 3365 | 61.947 | 0.000 | 0.000 | 0.000 | 1.480 | 0.106 | $2.506 |
| KTQNGSGSPHSKAQAPAT | 3366 | 61.934 | 0.000 | 0.169 | 0.015 | 0.696 | 0.197 | 127.420 |
| KTQNGSGSPHSKSQSQQQ | 3367 | 61.870 | 0.000 | 0.000 | 0.000 | 0.200 | 0.175 | 64.027 |
| KAEHGSGSPHSKAQNQQT | 3368 | 61.830 | 0.000 | 0.000 | 0.000 | 0.772 | 0.184 | 116.201 |
| KTEDRSGSPHSKAQNQQT | 3369 | 61.756 | 0.000 | 0.000 | 0.000 | 1.004 | 0.408 | 66.887 |
| KNCLGSGSPHSKAQNQQT | 3370 | 61.442 | 0.000 | 0.036 | 0.000 | 1.849 | 0.026 | 82.488 |
| KTDRGSGSPHSKAQNQQT | 3371 | 61.419 | 0.000 | 0.004 | 0.000 | 0.211 | 0.316 | 74.256 |
| KTQNGSGSPHSKAQQPPT | 3372 | 61.258 | 0.000 | 0.000 | 0.000 | 0.758 | 0.115 | 87.661 |
| KTVKGSGSPHSKAQDQQT | 3373 | 61.175 | 0.000 | 0.041 | 0.000 | 0.432 | 0.090 | 58.114 |
| KNADGSGSPHSKAQNQQT | 3374 | 60.944 | 0.000 | 0.000 | 0.000 | 1.239 | 0.085 | 104.503 |
| KTDKVSGSPHSKAQNQQT | 3375 | 60.935 | 0.000 | 0.015 | 0.000 | 0.765 | 0.128 | 146.657 |
| KTQTGSGSPHSKAQTQLT | 3376 | 60.846 | 0.160 | 8.992 | 0.000 | 0.000 | 0.000 | 55.640 |
| KTQNGSGSPHSKAQAPST | 3377 | 60.696 | 0.200 | 0.005 | 0.000 | 0.751 | 0.263 | 115.528 |
| KNCVGSGSPHSKAQNQQT | 3378 | 60.535 | 0.000 | 0.000 | 0.000 | 0.018 | 0.282 | 96.175 |
| KTQRDAGSPHSKAQNQQT | 3558 | 60.346 | 0.000 | 0.000 | 0.000 | 0.141 | 0.251 | 113.179 |
| KTVKDSGSPHSKAQNQQT | 3379 | 60.216 | 0.000 | 0.019 | 0.000 | 0.443 | 0.251 | 87.334 |
| KNALGSGSPHSKAQNQQT | 3380 | 60.014 | 0.000 | 0.003 | 0.000 | 0.682 | 0.213 | 137.222 |
| KVQNGSGSPHSKGQNQQT | 3381 | 60.001 | 0.000 | 0.000 | 0.031 | 0.264 | 0.157 | 68.532 |
| KTVNGGGSPHSKAQNQQS | 3559 | 59.871 | 0.062 | 0.020 | 0.000 | 0.080 | 0.185 | 61.847 |
| KTQQDGGSPHSKAQNQQT | 3560 | 59.865 | 0.000 | 0.000 | 0.116 | 1.435 | 0.789 | 87.522 |
| KTQSGGGSPHSKAQNQQN | 3561 | 59.801 | 0.000 | 0.000 | 0.000 | 0.722 | 0.039 | 87.761 |
| KTSNASGSPHSKAHNQQT | 3382 | 59.607 | 0.000 | 0.078 | 0.067 | 0.031 | 0.050 | 67.967 |
| KTQNGSGSPHSKAQNTYA | 3383 | 59.603 | 0.000 | 0.000 | 0.000 | 0.425 | 0.346 | 101.715 |
| KTQNGSGSPHSKSQNQHQ | 3384 | 59.438 | 0.000 | 0.099 | 0.000 | 0.111 | 0.108 | 76.025 |
| KTQNGGGSPHSKAQDKQT | 3562 | 59.322 | 0.000 | 0.000 | 0.000 | 0.000 | 0.093 | 50.764 |
| KTEFVSGSPHSKAQNQQT | 3385 | 59.306 | 0.000 | 0.000 | 0.000 | 0.196 | 0.276 | 69.788 |
| KTVNGSGSPHSKAQNHLT | 3386 | 59.239 | 0.133 | 0.034 | 0.000 | 0.000 | 0.156 | 70.786 |
| KTREQSGSPHSKAQNQQT | 3387 | 59.027 | 0.000 | 0.042 | 0.224 | 0.356 | 0.269 | 51.696 |
| KTQNGSGSPHSKAQQGMT | 3388 | 59.013 | 0.081 | 106.528 | 0.000 | 1.003 | 0.248 | 134.585 |
| KTQDGSGSPHSKALNKQT | 3389 | 58.992 | 0.000 | 0.267 | 0.000 | 0.000 | 0.056 | 74.626 |
| KTQQGGGSPHSKAQNPQT | 3563 | 58.924 | 0.000 | 0.202 | 0.000 | 0.000 | 0.126 | 53.992 |
| KQGEGSGSPHSKAQNQQT | 3390 | 58.752 | 0.000 | 0.000 | 0.000 | 0.000 | 0.151 | 135.300 |
| KTQNGTGSPHSKAPNQLT | 3564 | 58.738 | 0.000 | 0.000 | 0.000 | 0.229 | 0.035 | 86.939 |
| KTVNGSGSPHSKAQLQQT | 3391 | 58.681 | 0.315 | 0.465 | 0.045 | 0.529 | 0.333 | 81.20 |
| KTENGGGSPHSKAQYQQT | 3565 | 58.609 | 0.000 | 0.000 | 0.000 | 0.163 | 0.045 | 72.618 |
| KSQNGSGSPHSKTQSQQT | 3392 | 58.608 | 0.000 | 3.017 | 0.000 | 0.155 | 0.017 | 71.397 |
| KTVNGGGSPHSKAQHQQT | 3566 | 58.602 | 0.729 | 0.000 | 0.000 | 0.000 | 0.043 | 138.544 |
| KSEKGSGSPHSKAQNQQT | 3393 | 58.566 | 0.000 | 0.010 | 0.011 | 1.601 | 0.059 | 158.931 |
| KNVNGSGSPHSKAQNQQT | 3394 | 58.481 | 0.000 | 0.000 | 0.000 | 0.917 | 0.166 | 53.379 |
| KGGEGSGSPHSKAQNQQT | 3395 | 58.472 | 0.000 | 0.034 | 0.000 | 0.037 | 0.066 | 91.023 |
| KTQNGSGSPHSKAQRMST | 3396 | 58.435 | 0.192 | 0.037 | 0.000 | 1.707 | 0.882 | 53.414 |
| KTQNGSGSPHSKAQGQLT | 3397 | 58.418 | 0.000 | 0.005 | 0.010 | 0.569 | 0.192 | 102.631 |
| KEFVGSGSPHSKAQNQQT | 3398 | 58.374 | 0.000 | 0.046 | 0.000 | 0.088 | 0.326 | 128.675 |
| KTQQGSGSPHSKAQDRQT | 3399 | 58.258 | 1.393 | 0.230 | 0.219 | 0.000 | 0.045 | 53.981 |
| KSDKGSGSPHSKAQNQQT | 3400 | 58.248 | 0.000 | 0.000 | 0.000 | 0.076 | 0.166 | 146.566 |
| KTEQVSGSPHSKAQNQQT | 3401 | 58.247 | 0.000 | 0.000 | 0.000 | 0.000 | 0.081 | 88.487 |
| KTEHVSGSPHSKAQNQQT | 3402 | 58.228 | 0.000 | 0.024 | 0.000 | 0.433 | 0.141 | 71.410 |
| KTQNGSGSPHSKARDWQT | 3403 | 58.216 | 0.000 | 0.005 | 0.000 | 0.800 | 0.259 | 120.704 |
| KTENASGSPHSKAQNQQT | 3404 | 58.187 | 0.000 | 0.038 | 0.000 | 0.371 | 0.129 | 88.439 |
| KEVQGSGSPHSKAQNQQT | 3405 | 58.125 | 0.000 | 0.000 | 0.000 | 0.657 | 0.000 | 168.220 |
| KTQNGSGSPHSKAQNTHD | 3406 | 58.108 | 0.000 | 0.027 | 0.000 | 0.410 | 0.126 | 81.189 |
| KTQNGSGSPHSKAPNLQQ | 3407 | 58.022 | 0.000 | 0.044 | 0.000 | 1.548 | 0.243 | 55.714 |
| KTQNGSGSPHSKAQERST | 3408 | 58.021 | 0.000 | 0.011 | 0.005 | 0.829 | 0.409 | 87.656 |
| KTSNGSGSPHSKAQNYQT | 3409 | 57.894 | 0.000 | 0.082 | 0.000 | 0.000 | 0.110 | 63.681 |
| KTEYQSGSPHSKAQNQQT | 3410 | 57.891 | 0.000 | 0.000 | 0.000 | 0.076 | 0.075 | 57.620 |
| KTQNGSGSPHSKAQRTCT | 3411 | 57.863 | 0.000 | 0.140 | 0.129 | 1.855 | 1.716 | 90.146 |
| KTQNGSGSPHSKAQQGHT | 3412 | 57.769 | 0.024 | 0.000 | 0.000 | 0.281 | 0.154 | 99.262 |
| KNCWGSGSPHSKAQNQQT | 3413 | 57.756 | 0.000 | 0.000 | 0.000 | 0.000 | 0.092 | 59.888 |
| KTQNGSGSPHSKAQGAQT | 3414 | 57.627 | 0.000 | 0.000 | 0.000 | 0.594 | 0.161 | 95.696 |
| KTDVNSGSPHSKAQNQQT | 3415 | 57.593 | 0.000 | 0.000 | 0.000 | 0.000 | 0.331 | 66.127 |
| KSDQGSGSPHSKAQNQQT | 3416 | 57.592 | 0.000 | 0.000 | 0.000 | 0.844 | 0.128 | 107.342 |
| KTQNGSGSPHSKAQVPPT | 3417 | 57.316 | 0.000 | 0.012 | 0.000 | 0.257 | 0.200 | 90.220 |
| KTQNGSGSPHSKAQVQQQ | 3418 | 57.308 | 0.000 | 1.113 | 0.000 | 0.000 | 0.113 | 61.957 |
| KTQNGSGSPHSKALMRQT | 3419 | 57.234 | 0.060 | 0.036 | 0.100 | .798 | 0.517 | 81.332 |
| KTQNGSGSPHSKAQYSVT | 3420 | 57.130 | 0.000 | 0.093 | 0.000 | 1.235 | 0.302 | 60.023 |
| KNSQGSGSPHSKAQNQQT | 3421 | 57.101 | 0.000 | 0.052 | 0.000 | 0.083 | 0.074 | 97.381 |
| KTQNGSGSPHSKVPNLQT | 3422 | 57.046 | 0.000 | 0.029 | 0.000 | 0.459 | 0.082 | 50.474 |
| KAQNGSGSPHSKAQSQQQ | 3423 | 56.976 | 0.000 | 0.000 | 0.000 | 0.000 | 0.000 | 57.052 |
| KTQNGSGSPHSKAQAQTT | 3424 | 56.924 | 0.000 | 0.000 | 0.000 | 1.239 | 0.438 | 75.250 |
| KTQNGSGSPHSKAQKTLT | 3425 | 56.844 | 0.000 | 0.017 | 0.009 | 1.800 | 1.400 | 66.415 |
| KTVNGSGSPHSKAQNQWT | 3426 | 56.823 | 0.000 | 0.000 | 0.299 | 0.000 | 0.219 | 69.906 |
| KTQNGSGSPHSKAQLHHT | 3427 | 56.815 | 0.025 | 0.000 | 0.010 | 0.712 | 0.368 | 58.418 |
| KTEQTSGSPHSKAQNQQT | 3428 | 56.683 | 0.000 | 0.045 | 0.000 | 0.792 | 0.430 | 59.360 |
| KTQNGSGSPHSKAQNQQQ | 3429 | 56.630 | 0.000 | 0.062 | 0.123 | 0.099 | 0.056 | 76.742 |
| KNSQGSGSPHSKAQNQQT | 3430 | 56.621 | 0.000 | 0.028 | 0.000 | 0.308 | 0.162 | 101.942 |
| KTQPMEGSPHSKAQNQQT | 3567 | 56.560 | 0.000 | 0.000 | 0.000 | 1.824 | 0.371 | 89.951 |
| KTQNGSGSPHSKAQGHHT | 3431 | 56.559 | 0.000 | 0.000 | 0.000 | 0.632 | 0.117 | 71.050 |
| KTDRTSGSPHSKAQNQQT | 3432 | 56.466 | 0.000 | 0.000 | 0.000 | 0.062 | 0.160 | 148.498 |
| KTQNGSGSPHSKAQSKVT | 3433 | 56.373 | 0.000 | 0.050 | 0.014 | 1.021 | 0.390 | 76.115 |
| KEVVGSGSPHSKAQNQQT | 3434 | 56.371 | 0.000 | 0.000 | 0.000 | 0.000 | 0.323 | 116.964 |
| KTQNGSGSPHSKAQLPST | 3435 | 56.238 | 0.005 | 4.258 | 0.001 | 1.040 | 0.185 | 84.918 |
| KTQNGSGSPHSKAQGKQT | 3436 | 56.158 | 0.000 | 0.000 | 0.000 | 0.887 | 0.088 | 110.132 |
| KTEPTSGSPHSKAQNQQT | 3437 | 56.134 | 0.000 | 0.000 | 0.000 | 0.061 | 0.527 | 43.397 |
| KTVNGGGSPHSKSQNQQT | 3568 | 56.114 | 0.116 | 0.000 | 0.000 | 0.00 | 0.040 | 70.548 |
| KTQNGSGSPHSKAQAQHT | 3438 | 56.047 | 0.000 | 0.000 | 212.32 | 0.88″ | 0.890 | 81.908 |
| KTQNGSGSPHSKAQHGLT | 3439 | 55.999 | 0.000 | 0.000 | 0.101 | 1.913 | 0.244 | 117.191 |
| KSELGSGSPHSKAQNQQT | 3440 | 55.997 | 0.000 | 0.005 | 0.000 | 0.881 | 0.239 | 120.521 |
| KTQNGSGSPHSKAQEMCT | 3441 | 55.916 | 0.000 | 0.000 | 0.000 | 0.078 | 0.448 | 81.959 |
| KTQNVSGSPHSKAQGQQT | 3442 | 55.870 | 0.000 | 0.191 | 0.000 | 0.592 | 0.040 | 87.211 |
| KTQNGGGSPHSKAQNQMT | 3569 | 55.778 | 0.000 | 0.000 | 0.000 | 0.866 | 0.012 | 73.177 |
| KTVNGSGSPHSKAQHLQT | 3443 | 55.739 | 0.091 | 0.036 | 0.000 | 0.062 | 0.409 | 62.743 |
| KTQRENGSPHSKAQNQQT | 3570 | 55.605 | 0.000 | 0.000 | 0.016 | 0.131 | 0.257 | 95.931 |
| KTQNGSGSPHSKTQNHQN | 3444 | 55.551 | 0.000 | 0.048 | 0.000 | 0.000 | 0.099 | 64.846 |
| KTQNGSGSPHSKAQPART | 3445 | 55.513 | 0.000 | 0.000 | 0.328 | 1.294 | 0.991 | 127.301 |
| KTVNGSGSPHSKAQSLQT | 3446 | 55.497 | 0.000 | 0.060 | 0.000 | 0.000 | 0.143 | 69.033 |
| KTQNGSGSPHSKSQSQLT | 3447 | 55.430 | 0.000 | 0.035 | 0.000 | 0.050 | 0.013 | 125.577 |
| KTQNGSASPHSKAHSQQT | 3571 | 55.293 | 0.000 | 0.000 | 0.000 | 0.000 | 0.166 | 66.252 |
| KTWQNSGSPHSKAQNQQT | 3448 | 55.245 | 0.000 | 0.000 | 0.000 | 0.111 | 0.265 | 114.258 |
| KTQNGSGSPHSKAQDRQS | 3449 | 55.137 | 1.146 | 0.016 | 0.106 | 0.644 | 0.086 | 55.701 |
| KTQNGSGSPHSKAQMPST | 3450 | 54.986 | 1.691 | 0.039 | 0.028 | 0.450 | 0.202 | 114.331 |
| KTNNGGGSPHSKAQNLQT | 3572 | 54.963 | 0.000 | 0.000 | 0.000 | 0.000 | 0.089 | 80.506 |
| KTQNGSGSPHSKAQGSLT | 3451 | 54.717 | 0.000 | 0.006 | 0.013 | 0.480 | 0.298 | 142.786 |
| KTEVTSGSPHSKAQNQQT | 3452 | 54.663 | 0.000 | 0.000 | 0.000 | 0.323 | 0.185 | 81.482 |
| KSQNGGGSPHSKAQYQQT | 3573 | 54.612 | 0.000 | 0.000 | 0.000 | 0.105 | 0.010 | 65.952 |
| KQVQGSGSPHSKSQNQQT | 3453 | 54.603 | 0.000 | 0.000 | 0.000 | 0.000 | 0.106 | 69.121 |
| KAVNVSGSPHSKAQNQQT | 3454 | 54.586 | 0.000 | 0.000 | 0.000 | 0.000 | 0.023 | 57.835 |
| KTVNGNGSPHSKSQNQQT | 3574 | 54.586 | 0.000 | 0.000 | 0.000 | 0.256 | 0.168 | 95.384 |
| KTDRNSGSPHSKAQNQQT | 3455 | 54.495 | 0.000 | 0.000 | 0.000 | 0.82 | 0.241 | 85.823 |
| KTQNGSGSPHSKAQVPAT | 3456 | 54.475 | 0.000 | 0.002 | 0.000 | 0.782 | 0.223 | 137.743 |
| KGVLGSGSPHSKAQNQQT | 3457 | 54.472 | 0.000 | 0.007 | 0.027 | 0.359 | 0.189 | 45.740 |
| KTLNGNGSPHSKAQNLQT | 3575 | 54.458 | 0.668 | 0.000 | 0.000 | 0.161 | 0.172 | 159.134 |
| KAQNGSGSPHSKAQDKQT | 3458 | 54.452 | 0.000 | 0.000 | 0.057 | 0.044 | 0.223 | 56.004 |
| KTSNGSGSPHSKAHYQQT | 3459 | 54.414 | 0.000 | 0.251 | 0.000 | 0.249 | 0.204 | 54.162 |
| KTQNGSGSPHSKAQVPST | 3460 | 54.366 | 0.000 | 1.001 | 0.000 | 0.202 | 0.139 | 117.223 |
| KTQNGSGSSHSKAQNQQT | 3576 | 54.292 | 1.709 | 1.870 | 1.287 | 1.075 | 0.458 | 67.731 |
| KTELRSGSPHSKAQNQQT | 3461 | 54.289 | 0.000 | 0.007 | 0.040 | 0.790 | 0.239 | 57.814 |
| KNQNGSGSPHSKAQNHQT | 3462 | 54.248 | 0.000 | 0.034 | 0.000 | 0.340 | 0.075 | 74.979 |
| KTVNGGGSPHSKAQNHQT | 3577 | 54.246 | 0.375 | 0.024 | 0.000 | 0.000 | 0.146 | 67.188 |
| KTQNGSGSPHSKARGEQT | 3463 | 54.207 | 0.025 | 0.006 | 0.005 | 0.309 | 0.327 | 128.098 |
| KTQNGGGSPHSKAQYQHT | 3578 | 54.188 | 0.000 | 0.000 | 0.000 | 0.000 | 0.223 | 82.256 |
| KTEDLSGSPHSKAQNQQT | 3464 | 54.156 | 0.000 | 0.000 | 0.000 | 1.193 | 0.132 | 70.198 |
| KTQNGSGSPHSKAPGQQT | 3465 | 54.071 | 0.065 | 0.000 | 0.004 | 0.542 | 0.179 | 73.440 |
| KTQPKNGSPHSKAQNQQT | 3579 | 53.824 | 0.000 | 0.032 | 0.000 | 0.115 | 0.178 | 77.458 |
| KTQNGSGSPHSKAQSLQQ | 3466 | 53.778 | 0.000 | 0.186 | 0.000 | 0.022 | 0.047 | 51.543 |
| KTQNGSGSPHSKRLEQQT | 3467 | 53.512 | 0.000 | 0.118 | 0.003 | 0.161 | 0.292 | 71.704 |
| KTERGSGSPHSKAQNQQT | 3468 | 53.475 | 0.000 | 0.030 | 0.000 | 1.416 | 0.175 | 85.368 |
| KTVNGSGSPHSKAPNQQT | 3469 | 53.444 | 0.833 | 2.206 | 0.006 | 0.156 | 0.178 | 58.080 |
| KTSNGSGSPHSKAQNQST | 3470 | 53.353 | 0.000 | 0.000 | 0.000 | 0.000 | 0.014 | 120.897 |
| KTQNGSGSPHSKAQKVQT | 3471 | 53.273 | 0.000 | 0.000 | 0.000 | 0.357 | 0.402 | 95.147 |
| KTEGQSGSPHSKAQNQQT | 3472 | 53.270 | 0.000 | 0.000 | 0.000 | 0.000 | 0.010 | 78.303 |
| KTQNGSGSPHSKAQNNDQ | 3473 | 53.226 | 0.000 | 0.000 | 0.000 | 0.593 | 0.046 | 59.664 |
| KTQNGSGSPHSKAQSVHT | 3474 | 53.226 | 0.000 | 0.004 | 0.000 | 0.446 | 0.217 | 76.110 |
| KTQNGSGSPHSKAQPLGT | 3475 | 53.049 | 0.015 | 0.004 | 0.001 | 0.515 | 0.222 | 68.656 |
| KTQNKEGSPHSKAQNQQT | 3580 | 53.006 | 0.000 | 0.029 | 0.000 | 0.177 | 0.111 | 64.520 |
| KTCNASGSPHSKAQNQQT | 3476 | 52.998 | 0.000 | 0.011 | 0.000 | 0.897 | 0.141 | 67.934 |
| KAQNGSGSPHSKAHNQET | 3477 | 52.973 | 0.000 | 0.030 | 0.000 | 0.035 | 0.058 | 71.809 |
| KTEGLSGSPHSKAQNQQT | 3478 | 52.891 | 0.000 | 0.000 | 0.020 | 0.104 | 0.155 | 104.529 |
| KTRDASGSPHSKAQNQQT | 3479 | 52.861 | 0.000 | 0.000 | 0.010 | 1.062 | 0.402 | 52.089 |
| KTSNGSGSPHSKAQNLQQ | 3480 | 52.843 | 0.000 | 0.000 | 1.605 | 0.178 | 0.214 | 74.823 |
| KTGNGSGSPHSKAQQQQT | 3481 | 52.809 | 0.000 | 0.000 | 0.000 | 0.000 | 0.012 | 98.291 |
| KTVNGGGSPHSKAQNLQT | 3581 | 52.788 | 0.000 | 0.031 | 0.000 | 0.000 | 0.165 | 83.215 |
| KTDRSSGSPHSKAQNQQT | 3482 | 52.737 | 0.000 | 0.000 | 0.000 | 0.995 | 0.085 | 123.421 |
| KTQNGSGSPHSKAQVRNT | 3483 | 52.735 | 0.000 | 0.101 | 0.011 | 0.230 | 0.423 | 68.893 |
| KTQNGSGSPHSKAPSNQT | 3484 | 52.680 | 1.494 | 4.762 | 0.003 | 0.330 | 0.208 | 87.951 |
| KTQNGSGSPHSKAQVGHT | 3485 | 52.624 | 0.000 | 0.000 | 0.006 | 0.535 | 0.192 | 106.448 |
| KNAQGSGSPHSKAQNQQT | 3486 | 52.516 | 0.000 | 0.000 | 0.000 | 0.165 | 0.198 | 117.939 |
| KAENGSGSPHSKAQNQQT | 3487 | 52.487 | 0.000 | 0.157 | 0.029 | 0.000 | 0.242 | 120.256 |
| KTQNGSGSPHSKAQRDQT | 3488 | 52.415 | 0.098 | 0.000 | 0.008 | 1.784 | 0.605 | 88.122 |
| KTQNGSGSPHSKAQMPNT | 3489 | 52.408 | 0.084 | 0.036 | 0.025 | 0.057 | 0.359 | 66.040 |
| KTVNGSGSPHSKSQNQQT | 3490 | 52.395 | 0.033 | 0.077 | 0.013 | 0.105 | 0.175 | 58.000 |
| KTQPAQGSPHSKAQNQQT | 3582 | 52.346 | 0.000 | 0.009 | 0.000 | 0.034 | 0.134 | 51.949 |
| KTQNGSGSPHSKARGLQT | 3491 | 52.275 | 0.000 | 0.000 | 0.036 | 1.235 | 1.425 | 169.881 |
| KTELGSGSPHSKAQNQQT | 3492 | 52.232 | 0.000 | 0.007 | 0.006 | 0.532 | 0.088 | 87.314 |
| KAETGSGSPHSKAQNQQT | 3493 | 52.219 | 0.000 | 0.047 | 0.581 | 0.009 | 0.188 | 132.940 |
| KTQNGSGSPHSKLQKQQT | 3494 | 52.144 | 0.615 | 0.477 | 1.071 | 1.113 | 0.429 | 61.833 |
| KTQNGSGSPHSKAPSLQT | 3495 | 52.137 | 0.041 | 1.614 | 0.002 | 0.902 | 0.222 | 70.363 |
| KTQNGSGSPHSKAQRDQT | 3496 | 51.897 | 0.069 | 0.014 | 0.040 | 0.867 | 0.554 | 102.317 |
| KTDVGSGSPHSKAQNQQT | 3497 | 51.849 | 0.000 | 0.007 | 0.000 | 0.385 | 0.560 | 115.774 |
| KTQNGSGSPHSKNRDQQT | 3498 | 51.830 | 0.000 | 0.008 | 0.000 | 0.480 | 0.138 | 100.300 |
| KSQNGSGSPHSKAPNLQT | 3499 | 51.812 | 0.000 | 0.256 | 0.000 | 0.085 | 0.139 | 59.270 |
| KTQNGSGSPHSKAQAKGT | 3500 | 51.727 | 0.048 | 0.016 | 0.000 | 0.271 | 0.525 | 104.917 |
| KTVNGSGSPHSKAQDKQT | 3501 | 51.580 | 0.428 | 0.000 | 0.069 | 0.041 | 0.063 | 69.225 |
| KTQNGGGSPHSKAQNPQA | 3583 | 51.574 | 0.000 | 0.000 | 0.000 | 0.192 | 0.000 | 102.792 |
| KTQNGSGSPHSKAQSAHT | 3502 | 51.569 | 0.068 | 0.070 | 0.000 | 0.589 | 0.249 | 79.498 |
| KTQNGNGSPHSKSQNQHT | 3584 | 51.379 | 0.000 | 0.054 | 0.000 | 0.000 | 0.082 | 56.614 |
| KTVPTSGSPHSKAQNQQT | 3503 | 51.348 | 0.013 | 0.000 | 0.000 | 1.017 | 0.338 | 102.651 |
| KTQDGSGSPHSKSQNHQT | 3504 | 51.307 | 0.000 | 0.000 | 0.000 | 0.000 | 0.269 | 63.174 |
| KTDVKSGSPHSKAQNQQT | 3505 | 51.296 | 0.000 | 0.000 | 0.000 | 0.515 | 0.224 | 53.601 |
| KAQNRSGSPHSKAQDQQT | 350€ | 51.262 | 0.000 | 0.000 | 0.000 | 0.000 | 0.036 | 54.631 |
| KTQNGSGSPHSKAQSTMQ | 3507 | 51.249 | 0.018 | 0.002 | 0.002 | 0.321 | 0.341 | 73.213 |
| KTVNASGSPHSKAQNQLT | 3508 | 51.249 | 0.000 | 0.000 | 0.000 | 0.000 | 0.268 | 99.559 |
| KTQNGSGSPHSKAQREMT | 3509 | 51.076 | 0.000 | 24.900 | 143.49 | 1.564 | 0.476 | 70.961 |
| KTVHGSGSPHSKAQSQQT | 3510 | 51.057 | 0.000 | 0.000 | 0.000 | 0.143 | 0.146 | 54.185 |
| KTQNGGGSPHSKSQNRQT | 3585 | 51.017 | 0.000 | 0.000 | 0.000 | 0.000 | 0.421 | 149.370 |
| KTQNGSGSPHSKAQYRAT | 3511 | 51.008 | 0.000 | 0.158 | 0.000 | 0.690 | 0.120 | 50.650 |
| KTQNGGGSPHSKAQRQQT | 3586 | 50.998 | 0.000 | 0.041 | 0.000 | 0.99 | 0.142 | 147.942 |
| KTEPMSGSPHSKAQNQQT | 3512 | 50.960 | 0.203 | 0.000 | 0.000 | 1.816 | 0.415 | 126.322 |
| KTQNGSGSPHSKNQWQQT | 3513 | 50.800 | 0.000 | 0.044 | 0.047 | 0.111 | 0.324 | 65.506 |
| KETAGSGSPHSKAQNQQT | 3514 | 50.762 | 0.000 | 0.027 | 0.000 | 1.706 | 0.054 | 212.795 |
| KTQNGSGSPHSKAQRMNT | 3515 | 50.686 | 0.000 | 108.747 | 0.019 | 0.943 | 0.264 | 97.975 |
| KNNLGSGSPHSKAQNQQT | 3516 | 50.670 | 0.000 | 0.019 | 0.000 | 0.406 | 0.121 | 102.408 |
| KTQNGSGSPHAKAQNHQT | 3517 | 50.667 | 0.211 | 0.140 | 0.051 | 0.101 | 0.090 | 80.603 |
| KTQQKNGSPHSKAQNQQT | 3587 | 50.587 | 0.000 | 0.000 | 0.000 | 0.000 | 0.751 | 75.547 |
| KTQNGSGSPHSYHVNQQT | 3588 | 50.486 | 0.000 | 0.056 | 0.059 | 0.528 | 0.275 | 179.489 |
| KTQNGSGSPHSKAGDSQT | 3518 | 50.457 | 0.614 | 0.236 | 0.008 | 1.062 | 0.071 | 74.355 |
| KTQNGSGSPHSKLKSQQT | 3519 | 50.368 | 0.000 | 0.296 | 0.000 | 1.796 | 1.096 | 95.240 |
| KTQNGSGSPHSKAQKQST | 3520 | 50.285 | 0.000 | 0.000 | 0.088 | 0.108 | 0.302 | 51.115 |
| KTEYNSGSPHSKAQNQQT | 3521 | 50.256 | 0.000 | 0.000 | 0.000 | 0.000 | 0.009 | 62.679 |
| KTQNGSGSPHSKAPSMQT | 3522 | 50.249 | 0.000 | 0.000 | 0.004 | 0.941 | 0.460 | 75.504 |
| EAQNGSGSPHSKAQNQQT | 3523 | 50.243 | 0.629 | 0.094 | 0.000 | 0.057 | 1.519 | 117.305 |
| KTQNGSGSPHSKASPRQT | 3524 | 50.227 | 0.088 | 0.005 | 0.068 | 1.761 | 0.530 | 67.241 |
| KTQNGSGSPHSKRMEQQT | 3525 | 50.177 | 0.000 | 0.000 | 0.000 | 1.327 | 0.208 | 81.769 |
| KTQNGSGSPHSKAQYQNT | 3526 | 50.099 | 0.000 | 0.008 | 0.000 | 0.017 | 0.119 | 71.846 |
| KTERVSGSPHSKAQNQQT | 3589 | 96.943 | 0.000 | 0.000 | 0.000 | 0.000 | 0.144 | 135.438 |
| KAEQGHDSPHKSGQNQQT | 1754 | 63.249 | 0.000 | 0.000 | 0.000 | 0.060 | 0.024 | 27.173 |

Table 23 provides the peptide sequence of 341 matured capsid variants, and the fold enrichment of these matured capsid variants relative to the AAV9 control that demonstrated a 75-fold or greater increase in expression in the brain of NHPs relative to the AAV9 control and had a fold-change in expression that was less than 2 relative to the AAV9 control in the liver and the DRG.

TABLE 23
NGS fold-enrichment of TTM-001 and TTM-002 matured AAV capsid variants in the
brain of NHPs

| | | Fold Enrichment relative to AAV9 | | | | | | |
|---|---|---|---|---|---|---|---|---|
| | SEQ | | | | | Liver | Liver | |
| | ID | Brain | DRG | Heart | Muscle | RNA | DNA | Brain |
| Sequence | NO: | (NHP) | (NHP) | (NHP) | (NHP) | (NHP) | (NHP) | (Mouse) |
| KTENRSGSPHSKAQNQQI | 3591 | 86.359 | 0.000 | 113.67 | 0.000 | 0.000 | 0.092 | 25.568 |
| KTIIGSGSPHSKAQNRHT | 3239 | 217.176 | 0.000 | 0.000 | 0.000 | 0.000 | 0.000 | 210.515 |
| KTFPGSGSPHSKVQNQQT | 3240 | 199.720 | 0.000 | 0.000 | 0.000 | 0.000 | 0.967 | 97.703 |
| KTEKMSGSPHSKAQNQQT | 3241 | 169.461 | 0.523 | 0.000 | 0.000 | 0.000 | 0.158 | 109.161 |
| KAINGHDSPHKSGQIRQT | 3606 | 108.510 | 0.000 | 23.908 | 0.000 | 0.132 | 0.261 | 8.862 |
| KTINGHDSPHKIGQNQHA | 3607 | 77.321 | 0.000 | 18.836 | 0.028 | 0.220 | 0.132 | 7.578 |
| KEINGRGSPHSKAQNQQT | 3527 | 134.390 | 0.239 | 0.000 | 0.000 | 0.000 | 0.232 | 52.311 |
| KTVNRNGSPHSKAQNQQT | 3528 | 133.016 | 0.000 | 0.416 | 0.000 | 0.000 | 0.000 | 85.361 |
| KAINGYDSPHKSGQKQQT | 3608 | 83.803 | 0.041 | 9.491 | 0.000 | 0.031 | 0.150 | 13.057 |
| KTVNGSGSPHSKARDQQT | 3242 | 124.789 | 0.123 | 0.039 | 0.312 | 0.569 | 0.454 | 132.137 |
| KTESGHDSPHKSGQNQQT | 3609 | 86.513 | 0.000 | 7.414 | 0.000 | 0.000 | 0.038 | 13.163 |
| KTINGHDSPHKSGQSVQT | 3610 | 75.748 | 0.010 | 6.808 | 0.000 | 0.165 | 0.058 | 9.321 |
| KTENGSGSPHSKAPNLQT | 3243 | 121.436 | 0.000 | 0.167 | 0.000 | 0.000 | 0.015 | 168.920 |
| KTEKTSGSPHSKAQNQQT | 3244 | 120.337 | 0.000 | 0.355 | 0.000 | 0.000 | 0.119 | 101.467 |
| TTINGHDSPHKSGQNQQT | 3611 | 108.963 | 1.512 | 3.445 | 0.869 | 0.659 | 1.109 | 14.788 |
| KTINGHESPHKSGRSQQT | 3612 | 97.106 | 0.000 | 3.329 | 0.022 | 0.000 | 0.181 | 9.378 |
| KTINGSGSPHSKAHVRQT | 3245 | 119.798 | 0.000 | 0.000 | 0.262 | 0.694 | 1.039 | 165.590 |
| KTVNGSGSPHSKAPNQHT | 3246 | 117.207 | 0.000 | 0.109 | 0.000 | 0.000 | 0.074 | 51.008 |
| KTEKISGSPHSKAQNQQT | 3247 | 116.603 | 0.000 | 0.000 | 0.000 | 0.000 | 0.426 | 102.978 |
| KTINGPGSPHSKAHNQQT | 3529 | 115.742 | 0.146 | 0.000 | 0.235 | 0.000 | 0.513 | $2.508 |
| KTINGHDSPHKSGQNKLE | 3613 | 76.204 | 0.000 | 1.430 | 0.000 | 0.015 | 0.031 | 12.419 |
| KTVNGSGSPHSKTQSQQT | 3248 | 115.086 | 0.000 | 0.726 | 0.000 | 0.000 | 0.340 | 63.248 |
| TTINGSGSPHSKAQNQQT | 3249 | 114.856 | 1.340 | 14.856 | 0.827 | 1.281 | 0.957 | 72.058 |
| KSINESGSPHSKAQNQQI | 3250 | 113.833 | 0.000 | 0.000 | 0.000 | 0.000 | 0.000 | 67.649 |
| KTINGHDSPHKTGQNQQK | 3614 | 77.562 | 0.000 | 1.056 | 0.000 | 0.000 | 0.000 | 6.379 |
| KTERTSGSPHSKAQNQQT | 3251 | 112.957 | 0.000 | 0.009 | 0.000 | 1.128 | 0.207 | 117.374 |
| KTINGSGSPHSKAQPAKT | 3252 | 111.472 | 0.331 | 0.000 | 1.089 | 0.044 | 1.796 | 215.275 |
| KTINGRGSPHKRGQNQQT | 3837 | 120.889 | 0.100 | 0.814 | 0.434 | 0.458 | 0.614 | 13.988 |
| KTINGSGSPHTKAQNPPT | 3592 | 147.061 | 0.000 | 0.727 | 0.000 | 0.000 | 0.000 | 34.425 |
| KTEKSSGSPHSKAQNQQT | 3253 | 107.470 | 0.000 | 0.016 | 0.014 | 0.977 | 0.179 | 100.177 |
| KAINGHDNPHKSGQNQQT | 3615 | 88.906 | 0.297 | 0.721 | 0.482 | 0.222 | 0.130 | 9.702 |
| KTSYGNGSPHSKAQNQQT | 3530 | 105.937 | 0.000 | 0.000 | 0.000 | 0.000 | 0.114 | 105.894 |
| KTINGQDSPHKSGQHQQA | 3616 | 85.657 | 1.127 | 0.579 | 0.000 | 0.193 | 0.557 | 5.582 |
| KTEKGSGSPHSKAQNQQT | 3254 | 105.614 | 0.053 | 0.031 | 0.000 | 0.586 | 0.169 | 84.653 |
| KTINGSGSPHSKSQTQQN | 3255 | 104.474 | 0.000 | 0.131 | 0.000 | 0.084 | 0.038 | 54.021 |
| KTERISGSPHSKAQNQQT | 3256 | 103.692 | 0.000 | 0.000 | 0.000 | 0.062 | 0.370 | 89.637 |
| KTERASGSPHSKAQNQQT | 3257 | 103.669 | 0.000 | 0.000 | 0.000 | 0.127 | 0.070 | 115.550 |
| KSINGHDSPHKSGQIQHT | 3617 | 87.598 | 0.000 | 0.480 | 0.000 | 0.714 | 0.347 | 13.872 |
| KELHGSGSPHSKAQNQQT | 3258 | 102.680 | 0.000 | 0.000 | 0.000 | 1.634 | 0.592 | 96.554 |
| KAINGSGSPHSKAQNLAT | 3259 | 101.954 | 0.000 | 10.954 | 8.655 | 0.298 | 0.239 | 116.685 |
| KTVNGSGSPHSKSQNQLT | 3260 | 101.327 | 0.000 | 0.035 | 0.000 | 0.000 | 0.025 | 80.716 |
| KAINGHDSPHKSGPRQQT | 3618 | 145.142 | 0.000 | 0.408 | 0.000 | 0.000 | 0.000 | 8.259 |
| KTVNGHDSPHKSGHTQQT | 3619 | 82.246 | 0.000 | 0.378 | 1.142 | 0.000 | 0.123 | 6.160 |
| KSINGHDSPHKSGQRQHT | 3620 | 80.132 | 0.000 | 0.357 | 0.000 | 0.000 | 0.000 | 9.851 |
| KTERNSGSPHSKAQNQQT | 3261 | 99.892 | 0.000 | 0.000 | 0.000 | 0.000 | 0.107 | 87.392 |
| KSLNGSGSPHTKAQNQQT | 3593 | 81.515 | 0.197 | 0.333 | 0.000 | 0.000 | 0.085 | 45.140 |
| KSVNGNGSPHSKAQNQQT | 3531 | 99.385 | 0.000 | 1.329 | 0.000 | 0.359 | 0.079 | 51.016 |
| KAINGHDSPHKSAQSQQT | 3621 | 95.204 | 0.146 | 0.310 | 0.000 | 0.699 | 0.058 | 14.595 |
| KSIYGHESPHKSGQNQQS | 3622 | 90.947 | 0.817 | 0.310 | 0.000 | 0.000 | 0.243 | 8.064 |
| KTENGSGSPHSKAQGQQT | 3262 | 99.253 | 0.000 | 0.208 | 0.000 | 0.128 | 0.099 | 81.459 |
| KTVNGHDSPHKSLQNQQT | 3623 | 112.925 | 0.000 | 0.301 | 0.059 | 0.000 | 0.322 | 16.726 |
| KTINGSGSPHGWVQNQQT | 3532 | 97.122 | 0.000 | 0.000 | 0.000 | 1.240 | 1.975 | 290.720 |
| KTINGHGSPHSKAQNPQT | 3838 | 83.478 | 0.000 | 0.288 | 0.219 | 0.000 | 0.260 | 11.001 |
| KTSNGYDSPHKSGQKQQT | 3624 | 77.001 | 0.032 | 0.286 | 0.000 | 0.000 | 0.016 | 8.813 |
| KTVNGHDSPHKSGRNQET | 3625 | 102.695 | 0.000 | 0.286 | 0.000 | 0.000 | 0.027 | 11.958 |
| KTTNGHDSPHKSGQTQLT | 3626 | 115.637 | 0.000 | 0.283 | 0.000 | 0.052 | 0.321 | 17.885 |
| KAINGHDSPHKSEKNQQT | 3627 | 77.103 | 0.000 | 0.274 | 0.000 | 0.000 | 0.000 | 26.868 |
| KTERVSGSPHSKAQNQQT | 3263 | 96.943 | 0.000 | 0.000 | 0.000 | 0.000 | 0.144 | 135.438 |
| KTINGSGSPHSKALNRQS | 3264 | 96.843 | 0.136 | 0.532 | 0.000 | 0.042 | 0.178 | 55.945 |
| KTERLSGSPHSKAQNQQT | 3265 | 95.857 | 0.000 | 0.004 | 0.005 | 0.126 | 0.260 | 102.372 |
| KIINGRDSPHKSGQDQQT | 3628 | 78.773 | 0.000 | 0.254 | 0.000 | 0.000 | 0.156 | 16.132 |
| KTDNGSGSPHSKAHNQQT | 3266 | 95.164 | 0.000 | 0.000 | 0.000 | 0.000 | 0.027 | 55.313 |
| KTFHGSGSPHSKTQNQQT | 3267 | 94.714 | 0.000 | 0.210 | 0.120 | 0.000 | 0.000 | 51.119 |
| KTISGHDSPHKTGHNQQT | 3629 | 92.490 | 0.000 | 0.233 | 0.057 | 0.730 | 0.000 | 8.823 |
| KTVNAHDSPHKSGQNQLT | 3630 | 79.137 | 0.000 | 0.233 | 0.178 | 0.753 | 0.045 | 29.254 |
| KTINGGGSPHSKAQTQQI | 3533 | 92.345 | 0.000 | 0.000 | 0.000 | 0.000 | 0.023 | 54.199 |
| KSINGYDSPHKSGQTQQT | 3631 | 79.227 | 1.817 | 0.226 | 0.000 | 0.000 | 1.148 | 4.497 |
| KTINGHESPHKSGQTQQI | 3632 | 86.089 | 0.000 | 0.222 | 0.000 | 0.000 | 0.024 | 3.989 |
| KTINGHDSPHKSGQSKQA | 3633 | 101.460 | 0.000 | 0.222 | 0.000 | 0.185 | 0.114 | 7.510 |
| KTSNGSGSPHSKAQNPPT | 3268 | 91.528 | 0.000 | 0.000 | 0.000 | 0.000 | 0.039 | 51.541 |
| ETINGSGSPHSKAQNLQT | 3269 | 90.969 | 0.221 | 1.023 | 0.197 | 0.179 | 0.813 | 107.216 |
| KTVHGNGSPHSKAQNQQT | 3534 | 90.073 | 0.000 | 0.000 | 0.000 | 0.000 | 0.304 | 97.003 |
| NTINGSGSPHSKAQNQQT | 3270 | 90.017 | 1.712 | 1.261 | 1.171 | 0.923 | 0.540 | 55.179 |
| KTINGGGSPHSKAQNQQC | 3535 | 89.301 | 0.219 | 0.000 | 0.000 | 0.287 | 0.319 | 53.840 |
| KTENMSGSPHSKAQNQQT | 3271 | 89.247 | 0.000 | 0.000 | 0.000 | 0.000 | 0.260 | 130.568 |
| KTENVSGSPHSKAQNQQT | 3272 | 88.506 | 0.000 | 0.000 | 0.000 | 0.964 | 0.112 | 108.591 |
| KTSSGSGSPHSKAQYQQT | 3273 | 87.304 | 0.000 | 0.000 | 0.000 | 0.000 | 0.299 | 58.143 |
| KTIDGGGSPHSKAQNKQT | 3536 | 85.019 | 0.000 | 0.000 | 0.000 | 0.000 | 0.477 | 55.517 |
| KTEKVSGSPHSKAQNQQT | 3274 | 84.558 | 0.000 | 0.022 | 0.000 | 0.873 | 0.424 | 112.185 |
| KAINGSGSPHSKAQDQET | 3275 | 84.080 | 0.000 | 0.000 | 0.000 | 0.194 | 0.027 | 87.637 |
| KTCNKSGSPHSKAQNQQT | 3276 | 83.992 | 0.000 | 0.000 | 0.165 | 0.283 | 0.000 | 119.496 |
| KTINGGGSPHSKAQNQLI | 3537 | 83.881 | 0.000 | 0.000 | 0.000 | 0.046 | 0.387 | 78.383 |
| KNINGGGSPHSKAQNQQT | 3538 | 83.083 | 0.000 | 0.042 | 0.000 | 0.000 | 0.000 | 75.913 |
| KTEHLSGSPHSKAQNQQT | 3277 | 83.080 | 0.000 | 0.000 | 0.012 | 0.021 | 0.189 | 69.494 |
| KAIIGHESPHKSGQNQQT | 3634 | 88.563 | 0.000 | 0.150 | 0.000 | 0.062 | 0.145 | 8.530 |
| KTINGHDSPHKTGQNQPP | 3635 | 77.357 | 0.000 | 0.149 | 0.000 | 0.000 | 0.096 | 8.865 |
| KAINGHDSPHKSGQSPQT | 3636 | 75.734 | 0.095 | 0.148 | 0.000 | 0.000 | 0.238 | 14.195 |
| KAEMGSGSPHSKAQNQQT | 3278 | 83.049 | 0.000 | 0.020 | 0.000 | 0.768 | 0.112 | 135.019 |
| KATNGSGSPHSKAQNHQT | 3279 | 82.627 | 0.000 | 0.176 | 0.000 | 0.155 | 0.057 | 66.207 |
| KTIKGNDSPHKSVQNQQT | 3637 | 85.986 | 0.000 | 0.135 | 0.000 | 0.263 | 0.000 | 8.603 |
| KAIKGSGSPHSKAQDQQT | 3280 | 82.258 | 0.000 | 0.000 | 0.000 | 0.108 | 0.000 | 85.178 |
| KTINGGGSPHSKSQNQLT | 3539 | 82.231 | 0.000 | 0.070 | 0.000 | 0.000 | 0.498 | 126.986 |
| KTEFGHDSPHKSGQNQQT | 3638 | 7.245 | 0.000 | 0.124 | 0.000 | 0.561 | 0.063 | 16.337 |
| KTINGHDSPHKSAQNYQT | 3639 | 30.375 | 0.000 | 0.124 | 0.000 | 0.097 | 0.123 | 19.443 |
| KTENGSASPHSKALNQQT | 3839 | 84.258 | 0.000 | 0.122 | 0.000 | 0.104 | 0.037 | 31.855 |
| KTINGCGSPHASGQNQQT | 3840 | 132.540 | 0.000 | 0.121 | 0.042 | 0.000 | 0.059 | 1.857 |
| KTINAHDSPHKIGQNHQT | 3640 | 106.832 | 0.000 | 0.121 | 0.000 | 0.000 | 0.231 | 5.074 |
| KTVNGNGSPHSKAQNKQT | 3540 | 81.481 | 0.000 | 0.000 | 0.000 | 0.000 | 0.122 | 69.455 |
| KTINGHESPHKSAQNRQT | 3641 | 95.531 | 0.000 | 0.113 | 0.000 | 0.130 | 0.082 | 4.815 |
| KTINGSGSPHSKGHWQQT | 3281 | 81.434 | 0.000 | 0.000 | 0.000 | 0.000 | 1.011 | 65.252 |
| KTINGHDSPHKSGQNQQG | 3642 | 85.113 | 0.000 | 0.107 | 0.000 | 0.000 | 0.017 | 10.555 |
| KTIKGQDSPHKIGQNQQT | 3643 | 110.357 | 0.000 | 0.103 | 0.058 | 0.166 | 0.135 | 11.829 |
| KTDKTSGSPHSKAQNQQT | 3282 | 81.430 | 0.000 | 0.000 | 0.000 | 1.362 | 0.291 | 169.515 |
| KTVNGHDSPHKSGQNHLT | 3644 | 81.516 | 0.000 | 0.100 | 0.017 | 0.000 | 0.028 | 16.096 |
| KTFKGSGSPHSKAPNQQT | 3283 | 80.890 | 0.000 | 0.000 | 0.000 | 0.000 | 0.017 | 71.144 |
| KSINGHDSPHKSGQYQHT | 3645 | 88.195 | 0.000 | 0.099 | 0.000 | 0.000 | 0.149 | 14.485 |
| KTINGNDSPHKSVQNHQT | 3646 | 120.002 | 0.000 | 0.099 | 0.788 | 0.000 | 0.000 | 7.920 |
| KTVNGSGSPHSKAQNQLI | 3284 | 80.509 | 0.000 | 0.000 | 0.000 | 0.000 | 0.166 | 71.156 |
| KTINGSGSPHSKRPEQQT | 3285 | 80.418 | 0.000 | 0.013 | 0.000 | 0.149 | 0.361 | 50.319 |
| KTINGSGSPHSKAQRTMT | 3286 | 80.388 | 0.000 | 0.022 | 0.170 | 1.812 | 1.025 | 100.248 |
| KTITGHDSPHKSGQNQWT | 3647 | 81.658 | 0.000 | 0.090 | 0.000 | 0.936 | 0.000 | 7.744 |
| KINNGHDSPHKSVQNQHT | 3648 | 115.172 | 0.000 | 0.083 | 0.000 | 0.000 | 0.062 | 7.934 |
| KTEKASGSPHSKAQNQQT | 3287 | 80.285 | 0.000 | 0.041 | 0.000 | 0.000 | 0.26 | 90.390 |
| KTIDGHDSPHKSGQNQHA | 3649 | 91.058 | 0.000 | 0.082 | 0.000 | 0.000 | 0.000 | 10.781 |
| KSDQGSGSPHSKAQNQQT | 3288 | 80.076 | 0.000 | 0.000 | 0.000 | 0.993 | 0.124 | 151.911 |
| KTVNGHDSPHKSGQTRQT | 3650 | 133.276 | 0.251 | 0.080 | 0.093 | 0.034 | 0.129 | 7.174 |
| KTVNGHDSPHKSGQNLHT | 3651 | 88.080 | 0.000 | 0.080 | 0.000 | 0.000 | 0.039 | 11.363 |
| KAISGHDSPHKSGLNQQT | 3652 | 78.846 | 0.000 | 0.079 | 0.000 | 0.000 | 0.015 | 11.045 |
| KTEITSGSPHSKAQNQQT | 3289 | 79.620 | 0.000 | 0.163 | 0.000 | 0.332 | 0.074 | 76.686 |
| KTDKSSGSPHSKAQNQQT | 3290 | 79.470 | 0.055 | 0.012 | 0.000 | 1.437 | 0.367 | 141.351 |
| KAINGHDSPHKSAQNQET | 3653 | 90.402 | 0.000 | 0.073 | 0.000 | 0.746 | 0.000 | 10.674 |
| KTITGHDSPHKSGQHLQT | 3654 | 137.945 | 0.000 | 0.072 | 0.000 | 0.000 | 0.000 | 4.187 |
| KTIDGSGSPHSKAQNQQH | 3291 | 79.090 | 0.000 | 0.000 | 0.000 | 0.136 | 0.049 | 57.914 |
| KTVNGNGSPHSKAQNQHT | 3541 | 78.849 | 0.000 | 0.000 | 0.000 | 0.000 | 0.045 | 54.086 |
| KNINGSGSPHSKAQNQQT | 3292 | 78.445 | 0.000 | 0.000 | 0.000 | 0.571 | 0.177 | 89.719 |
| KTINGHDSPHKSRLNQPT | 3655 | 92.883 | 0.000 | 0.070 | 0.050 | 0.904 | 1.075 | 5.598 |
| KTETHSGSPHSKAQNQQT | 3293 | 77.974 | 0.000 | 0.067 | 0.000 | 0.000 | 0.512 | 57.287 |
| KTVDGHDSPHKSGQKQQT | 3656 | 78.802 | 0.000 | 0.069 | 0.000 | 0.157 | 0.342 | 7.200 |
| KTINGQDSPHKSGQNQDT | 3657 | 82.075 | 0.000 | 0.067 | 0.000 | 0.225 | 0.144 | 9.626 |
| KTINGGGSPHSKALNQQN | 3542 | 77.822 | 0.000 | 0.131 | 0.000 | 0.000 | 0.274 | 69.884 |
| KTIEGHDSPHKSGRNQQT | 3658 | 75.838 | 0.000 | 0.065 | 0.017 | 0.000 | 0.079 | 7.818 |
| KTINGHDSPHKSGQNLLT | 3659 | 77.738 | 0.130 | 0.064 | 0.185 | 0.424 | 0.326 | 15.192 |
| KTINGHDSPHKSGQLVIT | 3660 | 76.781 | 0.089 | 0.064 | 0.000 | 0.338 | 0.475 | 11.323 |
| KTVNGHDSPHKSRQSQQT | 3661 | 76.458 | 0.000 | 0.063 | 0.000 | 0.000 | 0.021 | 8.136 |
| KTINGSGSPHSKALHQHT | 3294 | 77.502 | 0.000 | 0.052 | 0.041 | 0.000 | 0.188 | 68.196 |
| KTINGHDSPHKSGRTQET | 3662 | 81.599 | 0.000 | 0.062 | 0.000 | 0.000 | 0.137 | 7.270 |
| KTINGHDSPHKSVQTHQT | 3663 | 77.309 | 0.237 | 0.062 | 0.000 | 0.000 | 0.116 | 7.519 |
| KTINGTGSPHSKAQNHQI | 3543 | 77.089 | 0.171 | 0.000 | 0.000 | 0.000 | 0.166 | 54.281 |
| KTINGSGSPHSKAQHRIT | 3295 | 76.849 | 0.105 | 0.499 | 0.170 | 1.424 | 0.214 | 127.000 |
| KTINGSGSPHSKAQYIHT | 3296 | 76.170 | 0.000 | 0.014 | 0.033 | 1.523 | 0.168 | 59.649 |
| KTSNGHDSPHKSGQNQPA | 3664 | 75.834 | 0.000 | 0.056 | 0.000 | 0.000 | 0.000 | 8.501 |
| KTEGKHDSPHKSGQNQQT | 3665 | 98.384 | 0.000 | 0.056 | 0.000 | 0.000 | 0.000 | 10.345 |
| KTENISGSPHSKAQNQQT | 3297 | 76.072 | 0.000 | 0.000 | 0.000 | 0.115 | 0.132 | 83.118 |
| KVINGHDSPHKSGQTQQT | 3666 | 91.665 | 0.000 | 0.055 | 1.526 | 0.311 | 0.000 | 7.391 |
| KTIIGGGSPHSKAHNQQT | 3544 | 75.872 | 0.000 | 0.050 | 0.000 | 0.000 | 0.235 | 65.492 |
| KTINGPDSPHKIGQNQQS | 3667 | 85.726 | 0.000 | 0.055 | 0.171 | 0.000 | 0.063 | 10.055 |
| KTINGSGSPHSKAQKFET | 3298 | 75.788 | 0.000 | 0.000 | 0.028 | 0.108 | 0.093 | 65.588 |
| KTSNESGSPHSKAQNHQT | 3299 | 75.720 | 0.000 | 0.000 | 0.000 | 0.169 | 0.217 | 70.590 |
| KTINGSGSPHSKAQFPST | 3300 | 75.677 | 0.000 | 0.004 | 0.000 | 0.849 | 0.127 | 119.712 |
| KTERPSGSPHSKAQNQQT | 3301 | 75.669 | 0.000 | 0.029 | 0.000 | 0.000 | 0.156 | 73.894 |
| KAVNGHDSPHKSVQNQQT | 3668 | 81.051 | 0.448 | 0.051 | 0.000 | 0.665 | 0.091 | 11.288 |
| KTINGNGSPHSKAQNPLT | 3545 | 75.269 | 0.000 | 0.000 | 0.000 | 0.366 | 0.000 | 53.583 |
| KSIKGNGSPHSKAQNQQT | 3546 | 75.196 | 0.000 | 0.000 | 0.000 | 0.000 | 0.000 | 90.251 |
| KTINGHDSPHKSRQDQHT | 3669 | 75.595 | 0.000 | 0.049 | 0.118 | 0.030 | 0.045 | 8.540 |
| KAINGPDSPHKSGQKQQT | 3670 | 78.213 | 0.464 | 0.047 | 0.000 | 0.323 | 0.162 | 10.395 |
| KTINGHDSPHKSRQSQHT | 3671 | 88.544 | 0.499 | 0.046 | 0.000 | 0.059 | 0.032 | 8.324 |
| KTIYGHDSPHKSVQNQLT | 3672 | 92.381 | 0.000 | 0.043 | 0.000 | 0.103 | 0.016 | 12.323 |
| KTVNGHDSPHKSGQNLLT | 3673 | 83.969 | 0.114 | 0.040 | 0.023 | 0.000 | 0.035 | 18.894 |
| KTESAHDSPHKSGQNQQT | 3674 | 80.810 | 0.000 | 0.039 | 0.000 | 0.000 | 0.000 | 13.338 |
| KTENKSGSPHSKAQNQQT | 3594 | 103.854 | 0.000 | 0.037 | 0.000 | 0.000 | 0.119 | 31.182 |
| KTTNGQDSPHKSGQNQQS | 3675 | 92.419 | 0.000 | 0.037 | 0.043 | 0.000 | 0.079 | 7.592 |
| KTDKGSGSPHSKAQNQQT | 3595 | 94.572 | 0.000 | 0.037 | 0.000 | 0.951 | 0.367 | 47.888 |
| KTIDGHDSPHKSGRNQQI | 3676 | 80.240 | 0.000 | 0.037 | 0.000 | 0.040 | 0.144 | 10.363 |
| KTINGYDSPHKSGQYQHT | 3677 | 81.534 | 0.000 | 0.036 | 0.000 | 0.000 | 0.000 | 10.524 |
| KTDNGHDSPHKSRQNQQT | 3678 | 105.312 | 0.000 | 0.033 | 0.000 | 0.000 | 0.018 | 7.931 |
| KTINGHDSPHKSWVRQQT | 3679 | 125.537 | 0.000 | 0.033 | 0.000 | 0.291 | 0.174 | 11.687 |
| KTINGHESPHKSGQNQHS | 3680 | 92.248 | 0.000 | 0.032 | 0.012 | 0.090 | 0.088 | 9.720 |
| KTVNGHDSPHKIGHNQQT | 3681 | 120.985 | 0.000 | 0.029 | 0.000 | 0.000 | 0.009 | 10.167 |
| KTCNGHDSPHKSGRNQQT | 3682 | 94.616 | 0.000 | 0.025 | 0.000 | 0.000 | 0.128 | 12.496 |
| KTINGNGSPHSKAQNHQA | 3841 | 88.274 | 0.000 | 0.024 | 0.000 | 0.000 | 0.041 | 36.754 |
| KNVVGHDSPHKSGQNQQT | 3683 | 75.330 | 0.000 | 0.024 | 0.000 | 0.063 | 0.049 | 8.077 |
| KTELWHDSPHKSGQNQQT | 3684 | 85.323 | 0.057 | 0.020 | 0.000 | 0.000 | 0.243 | 9.915 |
| KTELRHDSPHKSGQNQQT | 3685 | 98.098 | 0.000 | 0.019 | 0.000 | 0.000 | 0.007 | 6.588 |
| KTINGHDSPHKSNAWQQT | 3686 | 84.825 | 0.000 | 0.016 | 0.000 | 0.000 | 0.132 | 15.788 |
| KTDAGHDSPHKSGQNQQT | 3687 | 88.924 | 0.000 | 0.013 | 0.000 | 1.076 | 0.070 | 18.107 |
| KTEVGHDSPHKSGQNQQT | 3688 | 112.457 | 0.000 | 0.011 | 0.000 | 0.000 | 0.138 | 13.125 |
| KTESRHDSPHKSGQNQQT | 3689 | 81.766 | 0.000 | 0.011 | 0.000 | 0.052 | 0.036 | 6.975 |
| KSELGHDSPHKSGQNQQT | 3690 | 107.059 | 0.000 | 0.005 | 0.000 | 0.000 | 0.055 | 13.285 |
| KTINGHDSPHKSGQSVPT | 3691 | 77.840 | 0.000 | 0.003 | 0.000 | 0.136 | 0.061 | 6.768 |
| KTINGHESPHKSGQNIQP | 3692 | 253.840 | 0.000 | 0.000 | 0.000 | 0.000 | 0.000 | 14.042 |
| KTEMKHDSPHKSGQNQQT | 3693 | 240.075 | 0.000 | 0.000 | 0.000 | 0.000 | 0.000 | 3.183 |
| KTINGHDSPHKSVQNHLN | 3694 | 196.758 | 0.000 | 0.000 | 0.000 | 0.000 | 0.000 | 14.557 |
| KTINGHDSPHKIGLDQQT | 3695 | 165.627 | 0.000 | 0.000 | 0.000 | 1.942 | 0.000 | 5.469 |
| KTSNASGSPHSKAQHQQT | 3596 | 165.206 | 0.000 | 0.000 | 0.000 | 0.000 | 0.082 | 40.558 |
| KTINGHDSPHKRGPDQQS | 3696 | 160.084 | 0.000 | 0.000 | 0.000 | 0.000 | 0.000 | 2.923 |
| KTINGMGSPHSKTQNQQT | 3842 | 158.728 | 0.000 | 0.000 | 0.000 | 0.000 | 0.638 | 47.809 |
| KTIKGHDSPHKSGESQQT | 3697 | 142.264 | 0.000 | 0.000 | 0.000 | 0.000 | 0.218 | 4.176 |
| KTEGWHDSPHKSGQNQQT | 3698 | 142.064 | 0.000 | 0.000 | 0.000 | 0.000 | 0.264 | 11.785 |
| KTINGHDSPHKHGQNHQT | 3699 | 141.405 | 0.191 | 0.000 | 0.000 | 0.000 | 0.000 | 10.214 |
| KTEQLHDSPHKSGQNQQT | 3700 | 138.345 | 0.000 | 0.000 | 0.000 | 0.000 | 0.000 | 12.606 |
| KTVNGTGSPHSKAQNQLT | 3843 | 137.639 | 0.000 | 0.000 | 0.000 | 0.000 | 0.277 | 48.950 |
| KTIIGHDSPHKSGQYQHT | 3701 | 131.825 | 0.000 | 0.000 | 0.000 | 0.000 | 0.211 | 5.762 |
| KTSNGHDSPHKSVQNKQT | 3702 | 130.640 | 0.000 | 0.000 | 0.000 | 0.172 | 0.039 | 11.850 |
| KIVNGQVSPHKSGQNQQT | 3703 | 129.649 | 0.000 | 0.000 | 0.000 | 0.000 | 0.031 | 16.942 |
| KTVNGHDSPHKSGQRQLT | 3704 | 129.641 | 0.000 | 0.000 | 0.000 | 0.000 | 0.487 | 20.145 |
| KTVNGHDSPHKIGQNQLT | 3705 | 128.582 | 0.000 | 0.000 | 0.499 | 0.027 | 0.199 | 20.957 |
| KTINGHDSPHKSGQIIVT | 3706 | 125.245 | 0.000 | 0.000 | 0.151 | 0.000 | 0.379 | 6.808 |
| KTEKIHDSPHKSGQNQQT | 3707 | 125.178 | 0.000 | 0.000 | 0.000 | 0.000 | 0.000 | 17.604 |
| KTENAHDSPHKSGQNQQT | 3708 | 124.477 | 0.000 | 0.000 | 0.000 | 0.000 | 0.062 | 15.805 |
| KIGNGHESPHKSGQNQQT | 3709 | 123.324 | 0.000 | 0.000 | 0.000 | 0.000 | 0.000 | 11.198 |
| KEVMGHDSPHKSGQNQQT | 3710 | 121.107 | 0.000 | 0.000 | 0.000 | 0.000 | 0.000 | 17.191 |
| KTEVKHDSPHKSGQNQQT | 3711 | 119.733 | 0.000 | 0.000 | 0.000 | 0.000 | 0.000 | 5.550 |
| KTINGYDSPHKSGQKQST | 3712 | 119.615 | 0.000 | 0.000 | 0.000 | 0.000 | 0.000 | 7.970 |
| KTIHGNGSPHSKAQNQET | 3844 | 117.388 | 0.000 | 0.000 | 0.000 | 0.000 | 0.000 | 38.874 |
| KYQVGHDSPHKSGQNQQT | 3713 | 112.797 | 0.000 | 0.000 | 0.000 | 0.000 | 0.542 | 9.335 |
| KTEAMHDSPHKSGQNQQT | 3714 | 111.765 | 0.000 | 0.000 | 0.000 | 0.000 | 0.000 | 16.142 |
| KTIKGDDSPHKSVQNQQT | 3715 | 109.397 | 0.000 | 0.000 | 0.000 | 0.000 | 0.000 | 19.125 |
| KTINGHDSPHKSVQSHQT | 3716 | 109.375 | 0.107 | 0.000 | 0.319 | 0.000 | 0.547 | 12.617 |
| KTINGHDSPHKSGQFVVT | 3717 | 108.725 | 0.000 | 0.000 | 0.000 | 0.124 | 0.406 | 10.179 |
| KTVNGHDSPHKSRQNLQT | 3718 | 107.496 | 0.205 | 0.000 | 0.000 | 1.934 | 0.062 | 8.616 |
| KATNGHNSPHKSGQNQET | 3719 | 106.806 | 0.000 | 0.000 | 0.000 | 0.000 | 0.000 | 10.566 |
| KAINGHDSPHKSAQNQQI | 3720 | 106.539 | 0.000 | 0.000 | 0.000 | 0.000 | 0.113 | 21.786 |
| KTEHGHDSPHKSGQNQQT | 3721 | 106.486 | 0.000 | 0.000 | 0.000 | 0.000 | 0.006 | 14.956 |
| KTVENHDSPHKSGQNQQT | 3722 | 106.468 | 0.000 | 0.000 | 0.000 | 0.000 | 0.156 | 9.246 |
| KTIYGHDSPHKSGQSQPT | 3723 | 106.431 | 0.000 | 0.000 | 0.000 | 0.155 | 0.137 | 6.562 |
| KTISGHESPHKSGQNEQT | 3724 | 105.740 | 0.000 | 0.000 | 0.000 | 0.378 | 1.384 | 9.156 |
| KAIIGHDSPHKSAQNQQT | 3725 | 105.292 | 0.000 | 0.000 | 0.000 | 0.000 | 0.553 | 16.793 |
| KAIDGHDSPHKSGQNQLT | 3726 | 104.701 | 0.331 | 0.000 | 0.000 | 0.201 | 0.638 | 16.109 |
| KTIMGHDSPHKSVQNQQT | 3727 | 104.683 | 0.000 | 0.000 | 0.000 | 0.000 | 0.000 | 8.029 |
| KEVGGHDSPHKSGQNQQT | 3728 | 103.896 | 0.000 | 0.000 | 0.000 | 0.000 | 0.000 | 16.899 |
| KTINGHDSPHKSAQNLLT | 3729 | 103.332 | 0.000 | 0.000 | 0.256 | 0.194 | 0.000 | 15.722 |
| KTEFTHDSPHKSGQNQQT | 3730 | 102.052 | 0.062 | 0.000 | 0.000 | 0.439 | 0.047 | 12.527 |
| KTINASGSPHSKAINQQT | 3597 | 101.122 | 0.233 | 0.000 | 0.000 | 0.000 | 0.145 | 47.196 |
| KAINGNGSPHKRGQNQQT | 3845 | 100.925 | 0.000 | 0.000 | 0.000 | 0.000 | 0.159 | 10.011 |
| KSEMGHDSPHKSGQNQQT | 3731 | 100.539 | 0.000 | 0.000 | 0.000 | 0.000 | 0.000 | 18.356 |
| KAQQGHDSPHKSGQNQQT | 3732 | 100.395 | 0.000 | 0.000 | 0.000 | 0.000 | 0.057 | 3.954 |
| KTEVMHDSPHKSGQNQQT | 3733 | 99.473 | 0.000 | 0.000 | 0.000 | 0.000 | 0.000 | 12.400 |
| KAINGHDSPHKSGQSLQT | 3734 | 99.310 | 0.058 | 0.000 | 1.439 | 0.254 | 0.056 | 17.323 |
| KTINGSGSPHSKAPNQQH | 3598 | 99.300 | 0.252 | 0.000 | 0.000 | 0.000 | 0.038 | 39.297 |
| KCGEGHDSPHKSGQNQQT | 3735 | 99.298 | 0.000 | 0.000 | 0.000 | 0.000 | 0.000 | 13.147 |
| KTVNGHDSPHKSAQNHQT | 3736 | 99.257 | 0.000 | 0.000 | 0.078 | 0.000 | 0.027 | 17.639 |
| KTVNGHDSPHKSGQTQLT | 3737 | 98.524 | 0.000 | 0.000 | 0.313 | 0.183 | 0.172 | 14.883 |
| KTNNGHDSPHKSGRNRQT | 3738 | 98.307 | 0.000 | 0.000 | 0.124 | 0.000 | 0.037 | 5.840 |
| KTCNEHDSPHKSGQNQQT | 3739 | 97.092 | 0.000 | 0.000 | 0.000 | 0.000 | 0.000 | 8.364 |
| KTINGHDSPHKYGQNEQT | 3740 | 96.960 | 0.000 | 0.000 | 0.000 | 0.000 | 0.000 | 4.613 |
| KASNRHDSPHKSGHNQQT | 3741 | 96.283 | 0.000 | 0.000 | 0.000 | 0.000 | 0.675 | 8.045 |
| KTINGNGSPHSKAPNLQT | 3846 | 95.963 | 0.000 | 0.000 | 0.000 | 0.000 | 0.247 | 36.341 |
| KTETKHDSPHKSGQNQQT | 3742 | 95.121 | 0.000 | 0.000 | 0.000 | 0.000 | 0.083 | 4.831 |
| KSINGHDSPHKSQQNQQT | 3743 | 94.479 | 0.000 | 0.000 | 0.000 | 0.000 | 1.696 | 9.633 |
| KTIGGHDSPHKSGQNQQI | 3744 | 94.420 | 0.000 | 0.000 | 0.000 | 0.000 | 0.333 | 19.324 |
| KTDPQHDSPHKSGQNQQT | 3745 | 93.931 | 0.000 | 0.000 | 0.000 | 0.906 | 0.019 | 11.749 |
| KTINRHDSPHKIVQNQQT | 3746 | 93.409 | 0.000 | 0.000 | 0.000 | 0.000 | 0.000 | 3.064 |
| KTEQYHDSPHKSGQNQQT | 3747 | 93.065 | 0.000 | 0.000 | 0.000 | 0.000 | 0.047 | 15.190 |
| KTINGHDSPHKSVQSKQT | 3748 | 92.445 | 0.000 | 0.000 | 0.078 | 0.000 | 0.047 | 4.263 |
| KELVGHDSPHKSGQNQQT | 3749 | 92.262 | 0.000 | 0.000 | 0.000 | 0.594 | 0.000 | 14.890 |
| KTENRHDSPHKSGQNQQT | 3750 | 91.675 | 0.000 | 0.000 | 0.000 | 0.000 | 0.000 | 13.282 |
| KELMGHDSPHKSGQNQQT | 3751 | 91.191 | 0.000 | 0.000 | 0.000 | 0.000 | 0.000 | 14.032 |
| KTINGNDSPHKIGHNQQT | 3752 | 91.183 | 0.000 | 0.000 | 0.117 | 0.000 | 0.270 | 11.095 |
| KTIKGGGSPHSKAQDQQT | 3847 | 91.172 | 0.000 | 0.000 | 0.000 | 0.064 | 0.085 | 49.580 |
| KTEGHHDSPHKSGQNQQT | 3753 | 89.922 | 0.000 | 0.000 | 0.000 | 0.000 | 0.000 | 23.929 |
| KTEGYHDSPHKSGQNQQT | 3754 | 89.891 | 0.000 | 0.000 | 0.000 | 0.000 | 0.000 | 15.116 |
| KTVNGHDSPHKSGQTQQI | 3755 | 89.801 | 0.000 | 0.000 | 0.657 | 0.000 | 0.542 | 11.543 |
| KTINGQDSPHKSGQNPLT | 3756 | 89.726 | 0.000 | 0.000 | 0.000 | 0.363 | 0.000 | 15.561 |
| KTVNASGSPHSKAQNHQT | 3599 | 89.467 | 0.091 | 0.000 | 0.000 | 0.045 | 0.252 | 39.024 |
| KTINGHDSPHKSGRDQKT | 3757 | 88.871 | 0.000 | 0.000 | 0.000 | 0.350 | 0.181 | 12.117 |
| KTINGHDSPHKSVHNQQN | 3758 | 88.715 | 0.089 | 0.000 | 0.081 | 0.000 | 0.143 | 10.787 |
| KTINGHDSPHKSGQWKRT | 3759 | 88.633 | 0.000 | 0.000 | 0.000 | 0.202 | 0.094 | 5.186 |
| KTIDGSGSPHSKAENRQT | 3600 | 87.993 | 0.092 | 0.000 | 0.000 | 0.139 | 0.054 | 40.629 |
| KNEIGHDSPHKSGQNQQT | 3760 | 87.758 | 0.000 | 0.000 | 0.000 | 0.000 | 0.055 | 14.110 |
| KAINGHDSPHKSGQSQQI | 3761 | 87.585 | 0.000 | 0.000 | 5.310 | 0.000 | 0.000 | 12.864 |
| KIINGHDSPHKSRQAQQT | 3762 | 86.966 | 0.000 | 0.000 | 0.000 | 0.000 | 0.000 | 9.193 |
| KTPNGHDSPHKSGQNQQI | 3763 | 86.683 | 0.000 | 0.000 | 0.000 | 0.000 | 0.109 | 21.278 |
| KITNGHDSPHKSGQTQQT | 3764 | 86.443 | 0.000 | 0.000 | 0.000 | 0.192 | 0.190 | 17.479 |
| KTINGHDSPHKSVQNHQI | 3765 | 86.395 | 0.000 | 0.000 | 0.000 | 0.000 | 0.000 | 9.148 |
| KTINGHDSPHKSKQNQQA | 3766 | 86.265 | 0.000 | 0.000 | 0.000 | 0.123 | 0.041 | 5.768 |
| KTINGHDSPHKSAQNQLN | 3767 | 86.153 | 0.000 | 0.000 | 0.000 | 0.050 | 0.019 | 15.587 |
| KTDITHDSPHKSGQNQQT | 3768 | 85.876 | 0.000 | 0.000 | 0.000 | 0.000 | 0.013 | 9.076 |
| KTVNGHDSPHKSGQTQPT | 3769 | 85.680 | 0.000 | 0.000 | 1.301 | 1.064 | 0.000 | 8.067 |
| KTEKFHDSPHKSGQNQQT | 3770 | 85.358 | 0.000 | 0.000 | 0.000 | 0.000 | 0.026 | 7.229 |
| KTDQGHDSPHKSGQNQQT | 3771 | 85.267 | 0.000 | 0.000 | 0.000 | 0.000 | 0.000 | 16.042 |
| KTINGHDSPHKLWINQQT | 3772 | 85.132 | 0.000 | 0.000 | 1.154 | 0.000 | 0.017 | 12.704 |
| KGINGPDSPHKSGQNQQT | 3773 | 85.080 | 0.000 | 0.000 | 0.084 | 0.000 | 0.054 | 13.750 |
| KSEIGHDSPHKSGQNQQT | 3774 | 84.789 | 0.000 | 0.000 | 0.000 | 0.000 | 0.013 | 15.955 |
| KTINGHDSPHKSVQKQLT | 3775 | 84.351 | 0.000 | 0.000 | 0.000 | 0.038 | 0.103 | 1.890 |
| KTINGHPSPHWKGQNQQT | 3848 | 84.153 | 0.000 | 0.000 | 0.000 | 0.000 | 0.058 | 3.280 |
| KTVNGHDSPHKSGRNQLA | 3776 | 83.858 | 0.000 | 0.000 | 0.000 | 0.000 | 0.132 | 21.252 |
| KTNNVHDSPHKSGQNQQS | 3777 | 83.697 | 0.000 | 0.000 | 0.000 | 0.176 | 0.000 | 7.117 |
| KTIKGSGSPHSKVQDQQT | 3601 | 83.077 | 0.000 | 0.000 | 0.034 | 0.000 | 0.107 | 21.001 |
| KSEKGHDSPHKSGQNQQT | 3778 | 82.982 | 0.000 | 0.000 | 0.000 | 0.000 | 0.105 | 16.662 |
| KWSAGHDSPHKSGQNQQT | 3779 | 82.949 | 0.000 | 0.000 | 0.000 | 0.000 | 0.211 | 12.499 |
| KELAGHDSPHKSGQNQQT | 3780 | 82.876 | 0.000 | 0.000 | 0.000 | 0.000 | 0.093 | 18.063 |
| KTINGHDSPHKMGRNQQS | 3781 | 82.787 | 0.000 | 0.000 | 0.000 | 0.000 | 0.000 | 6.467 |
| KTDQAHDSPHKSGQNQQT | 3782 | 82.402 | 0.000 | 0.000 | 0.141 | 0.000 | 0.000 | 13.397 |
| KTETQHDSPHKSGQNQQT | 3783 | 82.316 | 0.000 | 0.000 | 0.000 | 0.000 | 0.198 | 10.823 |
| KTEMTHDSPHKSGQNQQT | 3784 | 82.221 | 0.000 | 0.000 | 0.000 | 0.000 | 0.000 | 8.431 |
| KTINGHDSPHKSGISIQT | 3785 | 82.019 | 0.000 | 0.000 | 0.000 | 0.191 | 0.044 | 7.310 |
| KTDAVHDSPHKSGQNQQT | 3786 | 81.968 | 0.000 | 0.000 | 0.000 | 0.297 | 0.107 | 13.596 |
| KTSNGHDSPHKSVQNLQT | 3787 | 81.921 | 0.000 | 0.000 | 0.072 | 0.000 | 0.330 | 11.544 |
| KTEKYHDSPHKSGQNQQT | 3788 | 81.637 | 0.000 | 0.000 | 0.000 | 0.000 | 0.013 | 7.580 |
| KQTQGHDSPHKSGQNQQT | 3789 | 81.581 | 0.000 | 0.000 | 0.000 | 0.000 | 0.133 | 15.225 |
| KTINGHDSPHKMAHNQQT | 3790 | 81.329 | 0.000 | 0.000 | 0.000 | 0.000 | 0.094 | 15.949 |
| KAINGSGSPHSKAQTQQA | 3602 | 81.207 | 0.000 | 0.000 | 0.000 | 0.000 | 0.016 | 40.435 |
| KTINGHDSPHKHGQNQQN | 3791 | 81.065 | 0.000 | 0.000 | 0.000 | 0.000 | 0.000 | 4.110 |
| KGADGHDSPHKSGQNQQT | 3792 | 80.981 | 0.000 | 0.000 | 0.000 | 0.000 | 0.074 | 11.423 |
| KVGEGHDSPHKSGQNQQT | 3793 | 80.775 | 0.084 | 0.000 | 0.000 | 0.000 | 0.019 | 16.378 |
| KANEGHDSPHKSGQNQQT | 3794 | 80.470 | 0.000 | 0.000 | 0.000 | 0.000 | 0.000 | 12.818 |
| KTDTMHDSPHKSGQNQQT | 3795 | 80.364 | 0.000 | 0.000 | 0.000 | 0.000 | 0.000 | 13.166 |
| KTEAKSGSPHSKAQNQQT | 3603 | 80.088 | 0.192 | 0.000 | 0.000 | 0.000 | 0.613 | 47.130 |
| KTINGHDSPHKSVQSQQS | 3796 | 80.000 | 0.000 | 0.000 | 0.000 | 1.055 | 0.082 | 17.620 |
| KTIPGSGSPHSKAQNLQT | 3604 | 79.973 | 0.871 | 0.000 | 0.000 | 0.000 | 0.000 | 32.693 |
| KTCIAHDSPHKSGQNQQT | 3797 | 79.857 | 0.000 | 0.000 | 0.066 | 0.000 | 0.093 | 1.930 |
| KTINGHDSPHKSGQTVCT | 3798 | 79.730 | 0.000 | 0.000 | 0.000 | 0.050 | 0.030 | 7.873 |
| KELRGHDSPHKSGQNQQT | 3799 | 79.596 | 0.000 | 0.000 | 0.000 | 0.000 | 0.006 | 22.001 |
| KCQIGHDSPHKSGQNQQT | 3800 | 79.359 | 0.000 | 0.000 | 0.000 | 0.000 | 0.000 | 2.614 |
| KGVMGHDSPHKSGQNQQT | 3801 | 79.170 | 0.000 | 0.000 | 0.000 | 0.138 | 0.086 | 17.287 |
| KACDGHDSPHKSGQNQQT | 3802 | 78.648 | 0.000 | 0.000 | 0.000 | 0.000 | 0.128 | 17.767 |
| KTINGQDSPHKSGQYQQI | 3803 | 8.585 | 0.000 | 0.000 | 0.000 | 0.286 | 0.672 | 5.664 |
| KTINGHDSPHKSGQQIMT | 3804 | 78.534 | 0.000 | 0.000 | 0.000 | 0.000 | 0.058 | 7.067 |
| KTINGHDSPHKSRQNEQS | 3805 | 78.534 | 0.000 | 0.000 | 0.000 | 0.112 | 0.188 | 13.388 |
| KASNGHDSPHKSGLNHQT | 3806 | 78.451 | 0.000 | 0.000 | 0.000 | 0.000 | 0.000 | 17.975 |
| KTVNGHDSPHKSGQSQPT | 3807 | 8.309 | 0.000 | 0.000 | 0.000 | 0.000 | 0.231 | 10.627 |
| KNELGHDSPHKSGQNQQT | 3808 | 78.135 | 0.000 | 0.000 | 0.000 | 0.000 | 0.182 | 17.457 |
| KTETFHDSPHKSGQNQQT | 3809 | 78.070 | 0.000 | 0.000 | 0.000 | 0.782 | 0.007 | 4.693 |
| KAAEGHDSPHKSGQNQQT | 3810 | 77.793 | 0.000 | 0.000 | 0.000 | 0.000 | 0.060 | 13.552 |
| KGQNGHDSPHKSGQNQQT | 3811 | 77.770 | 0.000 | 0.000 | 0.000 | 0.107 | 0.056 | 13.618 |
| KNEFGHDSPHKSGQNQQT | 3812 | 77.740 | 0.000 | 0.000 | 0.000 | 0.000 | 0.029 | 16.318 |
| KTSIGYDSPHKSGQNQQT | 3813 | 77.730 | 0.000 | 0.000 | 0.000 | 0.057 | 0.178 | 4.831 |
| KIDNGHDSPHKSGQNLQT | 3814 | 77.565 | 0.504 | 0.000 | 0.000 | 0.000 | 0.000 | 16.184 |
| KTEGQHDSPHKSGQNQQT | 3815 | 77.423 | 0.000 | 0.000 | 0.000 | 0.000 | 0.748 | 20.310 |
| KTITGHDSPHKSRQDQQT | 3816 | 77.127 | 0.000 | 0.000 | 0.000 | 0.000 | 0.000 | 6.250 |
| KAEHGHDSPHKSGQNQQT | 3817 | 77.026 | 0.000 | 0.000 | 0.000 | 0.000 | 0.017 | 20.937 |
| KTINGDDSPHKSGQKQLT | 3818 | 76.968 | 0.000 | 0.000 | 0.000 | 0.163 | 0.014 | 15.820 |
| KCDQGHDSPHKSGQNQQT | 3819 | 76.887 | 0.000 | 0.000 | 0.000 | 0.193 | 0.013 | 27.317 |
| KEILGHDSPHKSGQNQQT | 3820 | 76.770 | 0.000 | 0.000 | 0.000 | 0.804 | 0.009 | 10.771 |
| KTIHGSGSPHSKAQNQAT | 3605 | 76.765 | 0.000 | 0.000 | 0.000 | 0.000 | 0.215 | 43.969 |
| KTERNHDSPHKSGQNQQT | 3821 | 76.751 | 0.000 | 0.000 | 0.000 | 0.000 | 0.000 | 14.979 |
| KAINGDDSPHKSGHNQQT | 3822 | 76.578 | 0.000 | 0.000 | 0.000 | 0.032 | 0.059 | 17.755 |
| KTSNGHNSPHKSGQNQET | 3823 | 76.515 | 0.000 | 0.000 | 0.000 | 0.000 | 0.000 | 4.764 |
| KTINGHDSPHKSGQMIHT | 3824 | 76.364 | 0.000 | 0.000 | 0.000 | 0.000 | 0.000 | 9.486 |
| KNAIGHDSPHKSGQNQQT | 3825 | 76.289 | 0.000 | 0.000 | 0.000 | 0.009 | 0.072 | 15.178 |
| KTDKFHDSPHKSGQNQQT | 3826 | 76.204 | 0.000 | 0.000 | 0.000 | 0.000 | 0.000 | 7.096 |
| KTEGFHDSPHKSGQNQQT | 3827 | 76.191 | 0.000 | 0.000 | 0.000 | 0.000 | 0.080 | 13.163 |
| KVINGHDSPHKSGRNHQS | 3828 | 75.961 | 0.000 | 0.000 | 0.000 | 0.000 | 0.000 | 13.568 |
| KTITGHDSPHKSVQNRQT | 3829 | 75.940 | 0.000 | 0.000 | 0.000 | 0.621 | 0.000 | 4.310 |
| KTPDMHDSPHKSGQNQQT | 3830 | 75.871 | 0.659 | 0.000 | 0.000 | 0.000 | 0.048 | 11.277 |
| KTINGHDSPHKSGQKMNT | 3831 | 75.820 | 0.000 | 0.000 | 0.000 | 0.000 | 0.167 | 6.373 |
| KTELQHDSPHKSGQNQQT | 3832 | 75.814 | 0.000 | 0.000 | 0.000 | 0.105 | 0.000 | 11.798 |
| KTIHGHDSPHKSGQSQQN | 3833 | 75.777 | 0.000 | 0.000 | 0.059 | 0.000 | 0.166 | 7.426 |
| KTEIGHDSPHKSGQNQQT | 3834 | 75.525 | 0.000 | 0.000 | 0.016 | 0.012 | 0.000 | 9.593 |
| KTINGHDSPHKSGQYQHA | 3835 | 75.308 | 0.000 | 0.000 | 0.000 | 0.000 | 0.017 | 17.081 |
| KTELYHDSPHKSGQNQQT | 3836 | 75.235 | 0.000 | 0.000 | 0.000 | 0.000 | 0.042 | 10.354 |

Table 24 provides the sequences of 216 matured capsid variants having a CV of less than 1 for the liver RNA samples isolated and a 10-fold or greater increase in expression relative to AAV9 in the liver of NHPs. These matured variants showed preferential transduction of the liver over other tissues as shown by a low value for fold-enrichment relative to AAV9 in the other tissues investigated including the brain, DRG, heart and muscle. As such. Table 24 provides TIM-001 and TTM-002 matured AAV capsid variants with liver-specific tropism. Across the peptides within the matured capsid variants in Table 24, approximately 175 of them comprised the sequence GSGSPH (SEQ ID NO: 4695) and further comprised additional modifications in the C-terminal region of the sequence.

TABLE 24
NGS fold-enrichment of TTM-001 and TTM-002 matured AAV capsid variants in the
liver of NHPs

| | | Fold Enrichment relative to AAV9 | | | | | |
|---|---|---|---|---|---|---|---|
| | SEQ | Liver | Liver | | | | |
| | ID | RNA | DNA | Brain | DRG | Heart | Muscle |
| Sequence | NO: | (NHP) | (NHP) | (NHP) | (NHP) | (NHP) | (NHP) |
| KTQRKSGSPHSKAQNQQT | 4011 | 119.659 | 1.439 | 0.000 | 0.000 | 0.000 | 0.000 |
| KTINGSGSPHSKAQARKT | 4681 | 96.557 | 3.644 | 0.000 | 0.000 | 0.000 | 0.000 |
| KYIVGSGSPHSKAQNQQT | 4682 | 94.721 | 4.480 | 0.000 | 0.000 | 0.000 | 0.000 |
| KTINGSGSPHSMYMNQQT | 4683 | 81.106 | 5.840 | 0.000 | 0.000 | 0.000 | 0.000 |
| KTINGSGSPHSKAFYRQT | 4684 | 77.541 | 3.577 | 0.000 | 0.000 | 0.000 | 0.000 |
| KTINGSGSPHSKLKRQQT | 4685 | 76.103 | 6.884 | 0.000 | 0.000 | 0.000 | 0.000 |
| KTINGSGSPHSKRHRQQT | 4686 | 73.225 | 4.648 | 0.000 | 0.000 | 0.000 | 0.000 |
| KTINGSGSPHSKAQKCIT | 4687 | 69.547 | 1.887 | 0.000 | 0.000 | 0.000 | 0.000 |
| KTINGSGSPHSKWRLQQT | 4688 | 68.083 | 2.037 | 0.000 | 0.000 | 0.000 | 0.000 |
| KTINGSGSPHSRCRNQQT | 4689 | 64.416 | 5.150 | 0.000 | 0.000 | 0.000 | 0.000 |
| KTINGSGSPHSFTCNQQT | 4690 | 63.936 | 2.155 | 0.000 | 0.000 | 0.000 | 271.289 |
| KTINGSGSPHSKFFIQQT | 4691 | 63.255 | 6.916 | 0.000 | 0.000 | 0.000 | 0.000 |
| KTINGSYSPHCLAQNQQT | 4012 | 62.942 | 0.309 | 0.000 | 0.000 | 0.000 | 0.000 |
| KTINGSGSPHSKAQYSRT | 4692 | 60.119 | 0.956 | 0.000 | 0.000 | 0.000 | 0.000 |
| KTINGSGSPHIVWQNQQT | 3849 | 58.021 | 6.056 | 0.000 | 0.000 | 0.000 | 0.000 |
| KTINGSGSPHSKYFMQQT | 3850 | 57.350 | 2.993 | 0.000 | 0.000 | 0.000 | 0.000 |
| KTINGSGSPHSKARQRQT | 3851 | 56.775 | 2.205 | 0.000 | 0.000 | 0.000 | 0.000 |
| KTINGSGSPHSCHQNQQT | 3852 | 56.242 | 8.562 | 0.000 | 0.000 | 0.000 | 0.000 |
| KTINGSGSPHEPWQNQQT | 3853 | 53.587 | 1.731 | 0.000 | 0.000 | 0.000 | 0.000 |
| KTINGSGSPHSKIRRQQT | 3854 | 53.528 | 1.388 | 0.000 | 0.000 | 0.000 | 0.000 |
| KTINGSGSPHVYYQNQQT | 3855 | 53.294 | 2.173 | 0.944 | 0.000 | 0.246 | 5.268 |
| KTINGSGSPHSLYWNQQT | 3856 | 53.262 | 0.000 | 0.000 | 0.000 | 0.000 | 0.000 |
| KTINGSGSPHSKPKRQQT | 3857 | 52.881 | 2.832 | 0.000 | 0.000 | 0.000 | 0.000 |
| KPRWGSGSPHSKAQNQQT | 3858 | 51.637 | 0.386 | 0.000 | 0.000 | 375.537 | 0.000 |
| KTINGSGSPHSKAFSWQT | 3859 | 51.304 | 1.805 | 0.000 | 0.000 | 0.000 | 0.000 |
| KTINGSGSPHSRFWNQQT | 3860 | 51.225 | 6.955 | 0.000 | 0.000 | 0.000 | 0.000 |
| KTINGSGSPHSKAQCLKT | 3861 | 49.565 | 1.453 | 0.000 | 0.000 | 0.000 | 0.000 |
| KTINGSGSPHSRMRNQQT | 3862 | 48.902 | 2.816 | 0.000 | 0.000 | 0.000 | 0.000 |
| KTINGSGSPHSVKKNQQT | 3863 | 48.475 | 3.908 | 0.000 | 0.000 | 0.000 | 0.000 |
| KTINGSGSPHSWAPNQQT | 3864 | 47.897 | 1.789 | 0.000 | 0.000 | 0.000 | 0.000 |
| KTINGSGSPHSLWKNQQT | 3865 | 45.796 | 4.010 | 0.000 | 0.000 | 0.000 | 0.000 |
| KTINGSGSPHSKARWQQT | 3866 | 45.017 | 2.377 | 0.000 | 0.000 | 0.000 | 0.000 |
| KTINGSGSPHSFRPNQQT | 3867 | 44.801 | 9.191 | 0.000 | 0.000 | 0.000 | 0.000 |
| KTINGSGSPHSKKVFQQT | 3868 | 43.747 | 4.480 | 0.000 | 0.000 | 0.000 | 0.000 |
| KTINGSLSPHEWAQNQQT | 4013 | 43.190 | 2.041 | 0.000 | 0.000 | 0.000 | 0.000 |
| KTINGSGSPHSYAFNQQT | 3869 | 43.037 | 1.742 | 0.000 | 0.000 | 0.000 | 0.000 |
| KTINHRISPHSKAQNQQT | 4014 | 42.998 | 1.876 | 0.000 | 0.000 | 0.000 | 0.000 |
| KTINGSGSPHSKACSRQT | 3870 | 42.696 | 2.468 | 0.000 | 0.000 | 0.000 | 0.000 |
| KTRRPSGSPHSKAQNQQT | 4015 | 42.374 | 2.384 | 0.000 | 0.000 | 0.000 | 0.000 |
| KYSAGSGSPHSKAQNQQT | 3871 | 41.310 | 1.824 | 0.000 | 0.000 | 0.000 | 0.000 |
| KTINGAYSPHRKAQNQQT | 4016 | 40.969 | 1.283 | 0.000 | 0.000 | 0.000 | 0.000 |
| KTINGSGSPHSKRLWQQT | 3872 | 40.932 | 4.801 | 0.000 | 0.000 | 0.000 | 0.000 |
| KTINGSGSPHSCSRNQQT | 3873 | 40.372 | 4.293 | 0.000 | 0.000 | 0.000 | 0.000 |
| KTINGSGSPHSRCPNQQT | 3874 | 39.529 | 4.890 | 0.000 | 0.000 | 0.000 | 0.000 |
| KTINGSGSPHSGACNQQT | 3875 | 39.163 | 3.215 | 0.000 | 0.000 | 0.000 | 4733.916 |
| KYYTGSGSPHSKAQNQQT | 3876 | 38.777 | 1.199 | 0.000 | 0.000 | 0.000 | 0.000 |
| KTINGSGSPHSKFRQQQT | 3877 | 38.665 | 3.260 | 0.000 | 0.000 | 0.000 | 0.000 |
| KTINGSGSPHSFPFNQQT | 3878 | 38.584 | 4.693 | 0.000 | 0.000 | 0.000 | 0.000 |
| KTINGSGSPHSFFGNQQT | 3879 | 38.088 | 6.101 | 0.000 | 0.000 | 0.000 | 0.000 |
| KTINGRRSPHGKAQNQQT | 4017 | 37.728 | 3.259 | 0.000 | 0.000 | 0.000 | 0.000 |
| KTINGSGSPHSMCQNQQT | 3880 | 37.209 | 1.348 | 0.000 | 0.000 | 0.000 | 0.000 |
| KTINGSGSPHSKLFWQQT | 3881 | 37.022 | 4.178 | 0.000 | 0.000 | 0.000 | 0.000 |
| KTINGSGSPHSKTRKQQT | 3882 | 36.010 | 2.858 | 0.000 | 0.000 | 0.000 | 0.000 |
| KTINGRTSPHRKAQNQQT | 4018 | 35.792 | 5.682 | 0.000 | 0.000 | 0.000 | 0.000 |
| KTINGSGSPHSGKRNQQT | 3883 | 35.120 | 5.396 | 0.000 | 0.000 | 0.000 | 0.000 |
| KTINGSGSPHSKAQNFKR | 3884 | 32.291 | 0.964 | 0.000 | 0.000 | 0.000 | 0.000 |
| KTINGSGSPHFYRQNQQT | 3885 | 31.724 | 9.342 | 0.000 | 0.000 | 0.000 | 0.000 |
| KTINGSRSPHAWAQNQQT | 4019 | 31.146 | 6.838 | 0.000 | 0.000 | 0.000 | 0.000 |
| KTINGSGSPHCRVQNQQT | 3886 | 31.043 | 1.203 | 0.000 | 0.000 | 0.000 | 0.000 |
| KTINGSGSPHYGIQNQQT | 3887 | 30.908 | 1.076 | 0.000 | 0.000 | 0.000 | 0.000 |
| KTINKCLSPHSKAQNQQT | 4020 | 30.667 | 5.097 | 0.000 | 0.000 | 0.000 | 0.000 |
| KTINGSGSPHSKAQRFKT | 3888 | 30.363 | 0.139 | 0.000 | 0.000 | 0.000 | 0.000 |
| KTINGSGSPHVNCQNQQT | 3889 | 30.010 | 6.122 | 0.000 | 0.000 | 0.000 | 0.000 |
| KTINGSGSPHSKPFRQQT | 3890 | 29.842 | 8.700 | 0.000 | 0.000 | 0.000 | 0.000 |
| KTINGSGSPHSLAWNQQT | 3891 | 29.015 | 4.746 | 0.000 | 0.000 | 0.000 | 0.000 |
| KTINGSGSPHSKRSYQQT | 3892 | 28.973 | 2.116 | 0.000 | 0.000 | 0.000 | 0.000 |
| KTINGSSSPHRCAQNQQT | 4021 | 28.887 | 1.829 | 0.000 | 0.000 | 0.000 | 0.000 |
| KTINGSGSPHWSYQNQQT | 3893 | 28.607 | 3.751 | 0.000 | 0.000 | 0.000 | 0.000 |
| KTINCRISPHSKAQNQQT | 4022 | 28.301 | 1.117 | 0.000 | 0.000 | 0.000 | 0.000 |
| KTINGSGSPHRWLQNQQT | 3894 | 28.147 | 6.882 | 0.000 | 0.000 | 0.000 | 0.000 |
| KTIFDCGSPHSKAQNQQT | 4023 | 27.844 | 1.602 | 0.000 | 0.000 | 0.000 | 0.000 |
| KTINGSGSPHPSCQNQQT | 3895 | 27.796 | 2.790 | 0.000 | 0.000 | 0.000 | 0.000 |
| KTINGSGSPHSSWLNQQT | 3896 | 27.318 | 3.271 | 0.000 | 0.000 | 0.000 | 0.000 |
| KTINSPRSPHSKAQNQQT | 4024 | 27.240 | 1.554 | 0.000 | 0.000 | 0.000 | 0.000 |
| KPRFGSGSPHSKAQNQQT | 3897 | 27.203 | 0.657 | 0.000 | 0.000 | 0.000 | 0.000 |
| KWLTGSGSPHSKAQNQQT | 3898 | 26.975 | 2.388 | 0.364 | 0.000 | 0.000 | 2578.486 |
| KTINGSGSPHSKRRAQQT | 3899 | 26.523 | 5.906 | 0.000 | 0.000 | 0.000 | 0.000 |
| KTINGSGSPHSKAQTCRT | 3900 | 26.472 | 6.369 | 0.000 | 0.000 | 0.000 | 0.000 |
| KTINGLDSPHRSRQNQQT | 4025 | 26.403 | 0.321 | 0.000 | 0.000 | 0.000 | 0.000 |
| KTINGSGSPHSKGCTQQT | 3901 | 26.068 | 0.529 | 0.000 | 0.000 | 0.000 | 0.000 |
| KTRTRSGSPHSKAQNQQT | 4026 | 25.852 | 6.894 | 0.000 | 0.000 | 0.000 | 0.000 |
| KTINGSGSPHVPWQNQQT | 3902 | 25.294 | 3.435 | 0.000 | 0.000 | 0.000 | 0.000 |
| KTINGSGSPHSKRYTQQT | 3903 | 25.267 | 9.412 | 0.000 | 0.000 | 0.000 | 0.000 |
| KTINGSISPHCPAQNQQT | 4027 | 24.932 | 0.556 | 0.000 | 0.000 | 0.000 | 0.000 |
| KTINGSGSPHSGCQNQQT | 3904 | 24.818 | 1.981 | 0.000 | 0.000 | 0.000 | 0.000 |
| KTINGSGSPHSFTPNQQT | 3905 | 24.227 | 1.036 | 0.000 | 0.000 | 0.000 | 0.000 |
| KTINGSGSPHSTTCNQQT | 3906 | 23.771 | 3.315 | 0.000 | 0.000 | 0.000 | 0.000 |
| KTINGSGSPHSKARMYQT | 3907 | 23.424 | 0.313 | 0.000 | 0.000 | 0.000 | 0.000 |
| KTINGLVSPHRKAQNQQT | 4028 | 23.417 | 2.739 | 0.000 | 0.000 | 0.000 | 0.000 |
| KTINGSGSPHPKRQNQQT | 3908 | 23.055 | 2.355 | 0.000 | 0.000 | 0.000 | 0.000 |
| KTINGSGSPHSKCFLQQT | 3909 | 22.987 | 1.434 | 0.000 | 0.000 | 0.000 | 0.000 |
| KTINGSGSPHWVPQNQQT | 3910 | 22.907 | 3.219 | 0.000 | 0.000 | 0.000 | 0.000 |
| KTINGSGSPHSFWSNQQT | 3911 | 22.857 | 1.345 | 0.000 | 0.000 | 0.000 | 0.000 |
| KRSYGSGSPHSKAQNQQT | 3912 | 22.474 | 2.841 | 0.000 | 0.000 | 0.000 | 0.000 |
| KYVFGSGSPHSKAQNQQT | 3913 | 22.232 | 2.346 | 0.000 | 0.000 | 0.000 | 0.000 |
| KTINGSGSPHSKFKNQQT | 3914 | 21.951 | 1.074 | 0.000 | 0.000 | 0.000 | 0.000 |
| KTINGSGSPHRIKQNQQT | 3915 | 21.720 | 3.064 | 0.000 | 0.000 | 0.000 | 0.000 |
| KTINGSGSPHSKAPRRQT | 3916 | 21.645 | 3.940 | 0.000 | 0.000 | 0.000 | 0.000 |
| KTINGSGSPHSFRYNQQT | 3917 | 21.097 | 4.148 | 0.000 | 0.000 | 0.000 | 0.000 |
| KTINGSGSPHSKMICQQT | 3918 | 21.036 | 0.144 | 0.000 | 0.000 | 0.000 | 0.000 |
| KTINGSGSPHLRWQNQQT | 3919 | 21.014 | 9.649 | 0.000 | 0.000 | 0.000 | 0.000 |
| KTINGSGSPHLPTQNQQT | 3920 | 20.704 | 3.127 | 0.000 | 0.000 | 0.000 | 0.000 |
| KTINGSGSPHSKWKSQQT | 3921 | 20.390 | 1.239 | 0.000 | 0.000 | 4.904 | 0.163 |
| KTINALRSPHSKAQNQQT | 4029 | 20.053 | 1.655 | 0.000 | 0.000 | 0.000 | 0.000 |
| KTINGSGSPHSYMRNQQT | 3922 | 20.007 | 2.293 | 0.000 | 0.000 | 0.000 | 0.000 |
| KTINGSGSPHSKAARRQT | 3923 | 19.998 | 6.633 | 0.000 | 0.000 | 0.000 | 0.000 |
| KTINGSGSPHLLCQNQQT | 3924 | 19.796 | 3.484 | 0.673 | 0.000 | 0.000 | 1.309 |
| KTINGSGSPHRCCQNQQT | 3925 | 19.084 | 2.213 | 0.000 | 0.000 | 0.000 | 0.000 |
| KTINGSGSPHLCVQNQQT | 3926 | 19.030 | 1.428 | 0.000 | 0.000 | 0.000 | 0.000 |
| KTINGSGSPHSKLTRQQT | 3927 | 19.004 | 2.712 | 0.000 | 0.000 | 0.000 | 0.000 |
| KTICGRGSPHSKAQNQQT | 4030 | 18.923 | 2.171 | 0.000 | 0.000 | 0.000 | 0.000 |
| KTTRKSGSPHSKAQNQQT | 4031 | 18.849 | 2.617 | 0.000 | 0.000 | 0.000 | 0.000 |
| KTINGSGSPHSKLCTQQT | 3928 | 18.674 | 1.269 | 0.000 | 0.000 | 0.000 | 0.000 |
| KKHLGSGSPHSKAQNQQT | 3929 | 18.521 | 0.658 | 0.000 | 0.000 | 0.000 | 0.000 |
| KTINGSGSPHSKIRGQQT | 3930 | 18.150 | 1.584 | 0.000 | 0.000 | 0.000 | 0.000 |
| KTMQRSGSPHSKAQNQQT | 4032 | 18.020 | 3.159 | 0.000 | 0.000 | 0.000 | 0.000 |
| KTINGSGSPHSYLVNQQT | 3931 | 17.766 | 1.267 | 0.000 | 0.000 | 0.000 | 0.000 |
| KTINGSGSPHQGCQNQQT | 3932 | 17.676 | 1.037 | 0.000 | 0.000 | 0.000 | 0.000 |
| KTINGSGSPHMAFQNQQT | 3933 | 17.644 | 0.542 | 0.000 | 0.000 | 0.000 | 0.000 |
| KTINGSGSPHSKACQFQT | 3934 | 17.640 | 8.562 | 0.000 | 0.000 | 0.000 | 9.605 |
| KTINGSGSPHSKWGLQQT | 3935 | 17.543 | 2.639 | 0.000 | 0.000 | 0.000 | 0.000 |
| KTINGSGSPHSKILRQQT | 3936 | 17.419 | 2.546 | 0.000 | 0.000 | 0.000 | 0.000 |
| KTINGSGSPHSFQINQQT | 3937 | 17.418 | 0.269 | 0.308 | 0.000 | 1.568 | 0.000 |
| KTINGSGSPHSKACISQT | 3938 | 17.371 | 0.240 | 0.000 | 0.000 | 0.000 | 0.000 |
| KTINGSGSPHSKAQTHRT | 3939 | 17.290 | 2.917 | 0.000 | 0.000 | 0.000 | 0.000 |
| KTINGSGSPHSKALRCQT | 3940 | 17.283 | 1.892 | 0.000 | 0.000 | 0.000 | 0.000 |
| KTINGSGSPHSKAFYIQT | 3941 | 17.172 | 0.239 | 0.000 | 0.000 | 0.000 | 0.000 |
| KTINGSGSPHSKAHARQT | 3942 | 17.075 | 1.800 | 0.000 | 0.000 | 0.000 | 0.000 |
| KTINGSGSPHSLCLNQQT | 3943 | 17.028 | 1.790 | 0.000 | 0.000 | 0.000 | 0.000 |
| KTINGSGSPHSKAFVRQT | 3944 | 16.935 | 1.985 | 0.000 | 0.000 | 0.000 | 0.000 |
| KPPLGSGSPHSKAQNQQT | 3945 | 16.897 | 0.805 | 0.000 | 0.000 | 0.000 | 0.000 |
| KTINGSGSPHRPWQNQQT | 3946 | 16.869 | 4.936 | 0.000 | 0.000 | 0.000 | 0.000 |
| KPARGSGSPHSKAQNQQT | 3947 | 16.793 | 1.391 | 0.000 | 0.000 | 0.000 | 0.000 |
| KTINGSGSPHRPRQNQQT | 3948 | 16.784 | 5.206 | 0.000 | 0.000 | 0.000 | 0.000 |
| KTINGSGSPHSCPQNQQT | 3949 | 16.701 | 1.776 | 0.000 | 0.000 | 0.000 | 0.000 |
| KTINGSGSPHSKAQFILT | 3950 | 16.650 | 4.208 | 0.000 | 0.000 | 0.000 | 0.000 |
| KTINGSSSPHWMAQNQQT | 4033 | 16.635 | 2.390 | 0.000 | 0.000 | 0.000 | 0.000 |
| KTRKRSGSPHSKAQNQQT | 4034 | 16.603 | 2.075 | 0.000 | 0.000 | 0.000 | 0.000 |
| KTINGSGSPHSVRYNQQT | 3951 | 16.390 | 1.413 | 0.000 | 0.000 | 0.000 | 0.000 |
| KSRRGSGSPHSKAQNQQT | 3952 | 16.131 | 1.446 | 0.000 | 0.000 | 0.000 | 0.000 |
| KTINGSGSPHSVRCNQQT | 3953 | 15.860 | 3.912 | 0.000 | 0.000 | 0.000 | 0.000 |
| KFFHGSGSPHSKAQNQQT | 3954 | 15.412 | 0.897 | 0.000 | 0.000 | 0.000 | 0.000 |
| KTINGSGSPHSKMPCQQT | 3955 | 15.343 | 1.063 | 0.000 | 0.000 | 0.000 | 0.000 |
| KTINGSGSPHSKKTSQQT | 3956 | 15.244 | 1.344 | 0.000 | 0.000 | 0.000 | 0.000 |
| KRYNGSGSPHSKAQNQQT | 3957 | 15.160 | 0.806 | 0.000 | 0.000 | 0.000 | 0.000 |
| KTINFTRSPHSKAQNQQT | 4035 | 14.908 | 3.751 | 0.000 | 0.000 | 0.000 | 0.000 |
| KTINGSGSPHSLPYNQQT | 3958 | 14.792 | 2.048 | 0.000 | 0.000 | 0.000 | 0.000 |
| KTINGSGSPHVYHQNQQT | 3959 | 14.770 | 1.733 | 0.000 | 0.000 | 0.000 | 0.000 |
| KTINGSGSPHSKAQSRKT | 3960 | 14.589 | 2.715 | 0.000 | 0.000 | 0.000 | 0.000 |
| KTINGSGSPHSYTRNQQT | 3961 | 14.535 | 1.986 | 0.000 | 0.000 | 0.000 | 0.000 |
| KTINNLRSPHSKAQNQQT | 4036 | 14.514 | 1.354 | 0.000 | 0.000 | 0.000 | 0.000 |
| KTINGRPSPHGKAQNQQT | 4037 | 14.442 | 0.705 | 0.000 | 0.000 | 0.000 | 0.000 |
| KTINWSRSPHSKAQNQQT | 4038 | 14.399 | 5.624 | 0.000 | 0.000 | 0.000 | 0.000 |
| KTINGSGSPHLVYQNQQT | 3962 | 14.196 | 1.045 | 0.000 | 0.000 | 0.000 | 0.000 |
| KTINGTRSPHKKAQNQQT | 4039 | 14.173 | 1.152 | 0.700 | 0.225 | 0.052 | 4.082 |
| KTINGSGSPHSKALRWQT | 3963 | 14.118 | 5.252 | 0.000 | 0.000 | 0.000 | 0.000 |
| KTINGSGSPHYRYQNQQT | 3964 | 14.107 | 1.027 | 0.000 | 0.000 | 0.000 | 0.000 |
| KTINGSGSPHSWLKNQQT | 3965 | 3.995 | 0.603 | 0.000 | 0.000 | 0.000 | 0.000 |
| KTINGSGSPHSKAQMQIT | 3966 | 13.990 | 0.371 | 0.000 | 0.000 | 0.000 | 0.000 |
| KTINGSVSPHCTAQNQQT | 4040 | 13.502 | 2.955 | 0.000 | 0.000 | 0.000 | 0.000 |
| KTINGSGSPHCPAQNQQT | 3967 | 13.359 | 1.409 | 0.000 | 0.000 | 0.000 | 0.000 |
| KTINGSGSPHSMCINQQT | 3968 | 13.114 | 0.392 | 0.000 | 0.000 | 0.000 | 0.000 |
| KTINGSGSPHSPPDNQQT | 3969 | 12.973 | 0.033 | 0.000 | 0.000 | 0.000 | 0.000 |
| KTINGSGSPHSKRNYQQT | 3970 | 12.781 | 5.528 | 0.000 | 0.000 | 0.000 | 0.000 |
| KTTRCSGSPHSKAQNQQT | 4041 | 12.639 | 8.168 | 0.000 | 0.000 | 0.000 | 0.000 |
| KTKLCSGSPHSKAQNQQT | 4042 | 12.570 | 2.139 | 0.000 | 0.000 | 0.000 | 0.000 |
| KTINLGCSPHSKAQNQQT | 4043 | 12.564 | 0.654 | 0.000 | 0.000 | 0.000 | 0.000 |
| KTINGSGSPHRWTQNQQT | 3971 | 12.490 | 0.844 | 0.000 | 0.000 | 0.000 | 0.000 |
| KTISGHDSPHISGQYQQT | 4044 | 12.395 | 0.420 | 0.000 | 0.000 | 0.074 | 1214.588 |
| KTINGSGSPHSKACRLQT | 3972 | 12.297 | 6.537 | 0.000 | 0.000 | 0.000 | 0.000 |
| KTINGSGSPHPRKQNQQT | 3973 | 12.249 | 3.248 | 0.000 | 0.000 | 0.000 | 0.000 |
| KTINGSGSPHSKCSVQQT | 3974 | 12.246 | 1.465 | 0.000 | 0.000 | 0.000 | 0.000 |
| KTINGSGSPHSKAQYVRT | 3975 | 12.239 | 3.275 | 0.000 | 0.000 | 0.000 | 0.000 |
| KTINGSGSPHSKARISQT | 3976 | 12.142 | 1.565 | 0.000 | 0.000 | 0.000 | 0.000 |
| KTINGRRSPHMKAQNQQT | 4045 | 12.136 | 3.510 | 0.000 | 0.000 | 0.000 | 0.000 |
| KTINGPWSPHRKAQNQQT | 4046 | 12.103 | 0.434 | 0.000 | 0.000 | 0.000 | 0.000 |
| KTINGSGSPHPFVQNQQT | 3977 | 12.091 | 1.286 | 0.000 | 0.000 | 0.000 | 0.000 |
| KTINGSGSPHSKLPKQQT | 3978 | 11.856 | 0.274 | 0.000 | 0.000 | 0.000 | 0.000 |
| KTINSCFSPHSKAQNQQT | 4047 | 11.847 | 1.016 | 0.000 | 0.000 | 0.000 | 0.000 |
| KTINGSGSPHSKSEQQQT | 3979 | 11.785 | 1.769 | 0.000 | 0.000 | 0.000 | 0.000 |
| KTINGSGSPHWVAQNQQT | 3980 | 11.703 | 3.634 | 0.000 | 0.000 | 0.000 | 0.000 |
| KTINGSGSPHSLYQNQQT | 3981 | 11.590 | 1.503 | 0.000 | 0.000 | 0.000 | 0.000 |
| KTINGSGSPHSKVRMQQT | 3982 | 11.572 | 1.835 | 0.000 | 0.000 | 0.000 | 0.000 |
| KTINYTRSPHSKAQNQQT | 3983 | 11.514 | 0.431 | 0.000 | 0.000 | 0.000 | 0.000 |
| KTIKRYGSPHSKAQNQQT | 4048 | 11.461 | 2.022 | 0.000 | 0.000 | 0.000 | 0.000 |
| KTINGSGSPHCALQNQQT | 4693 | 11.404 | 3.867 | 0.000 | 0.000 | 0.000 | 0.000 |
| KTINGSGSPHSSCINQQT | 3984 | 1.382 | 3.363 | 0.000 | 0.000 | 0.000 | 0.000 |
| KTINGSGSPHSKNSRQQT | 3985 | 11.280 | 1.093 | 0.000 | 0.000 | 0.000 | 0.000 |
| KTINGSGSPHSKRKRQQT | 3986 | 11.215 | 3.027 | 0.000 | 0.000 | 0.000 | 0.000 |
| KTINGSGSPHLCTQNQQT | 3987 | 11.176 | 2.489 | 0.000 | 0.000 | 0.000 | 0.000 |
| KTINGSGSPHSKAQSAKT | 3988 | 11.162 | 4.200 | 0.000 | 0.000 | 0.000 | 0.000 |
| KTINGSGSPHSTCLNQQT | 3989 | 11.132 | 4.762 | 0.000 | 0.000 | 0.000 | 0.000 |
| KTINGSGSPHSYARNQQT | 3990 | 11.131 | 0.996 | 0.000 | 0.000 | 0.000 | 0.000 |
| KTINGSGSPHSKQRPQQT | 3991 | 11.130 | 2.347 | 0.000 | 0.000 | 0.000 | 0.000 |
| KTINGSGSPHSKRVVQQT | 3992 | 11.094 | 1.639 | 0.000 | 0.000 | 0.000 | 0.000 |
| KRFSGSGSPHSKAQNQQT | 3993 | 11.024 | 1.358 | 0.000 | 0.000 | 0.000 | 0.000 |
| KTINGSGSPHKSGQNPQT | 3994 | 11.014 | 11.790 | 0.000 | 0.000 | 0.000 | 0.000 |
| KTINRYSSPHSKAQNQQT | 4049 | 10.926 | 1.544 | 0.000 | 0.000 | 0.000 | 0.000 |
| KTTGRSGSPHSKAQNQQT | 4050 | 10.863 | 0.126 | 0.000 | 0.000 | 0.000 | 0.000 |
| KTINGSGSPHSKALRHQT | 3995 | 10.774 | 4.532 | 0.000 | 0.000 | 0.000 | 0.000 |
| KTINGSGSPHSYYSNQQT | 3996 | 10.680 | 2.856 | 0.000 | 0.000 | 0.000 | 0.000 |
| KTINGSGSPHSLTCNQQT | 3997 | 10.658 | 2.214 | 0.490 | 0.000 | 0.163 | 1.398 |
| KTINGSGSPHSCQSNQQT | 3998 | 10.631 | 1.468 | 0.000 | 0.000 | 0.000 | 0.000 |
| KTINGSGSPHSKAQSIKT | 3999 | 10.544 | 1.355 | 0.000 | 0.000 | 0.000 | 0.000 |
| KYSMGSGSPHSKAQNQQT | 4000 | 10.478 | 1.587 | 0.000 | 0.000 | 0.000 | 0.000 |
| KTINGSGSPHSKAKGWQT | 4001 | 10.450 | 1.827 | 0.000 | 0.000 | 0.000 | 0.000 |
| KTIVGSGSPHSKPQNQQT | 4002 | 10.381 | 0.894 | 0.000 | 0.000 | 0.000 | 0.000 |
| KTINGSGSPHEPFQNQQT | 4003 | 10.322 | 3.715 | 0.000 | 0.000 | 0.000 | 0.000 |
| KPFLGSGSPHSKAQNQQT | 4004 | 10.318 | 1.328 | 0.000 | 0.000 | 0.000 | 0.000 |
| KTINGSGSPHSKCTSQQT | 4005 | 10.311 | 5.821 | 0.493 | 0.232 | 1.413 | 2.353 |
| KTINRQFSPHSKAQNQQT | 4051 | 10.275 | 4.480 | 0.000 | 0.000 | 0.000 | 0.000 |
| KTINGSGSPHSVFENQQT | 4006 | 10.218 | 0.224 | 0.000 | 0.000 | 0.000 | 0.000 |
| KTINGSGSPHSKAKKVQT | 4007 | 10.102 | 3.974 | 0.000 | 0.000 | 0.000 | 0.000 |
| KTINGSGSPHSKAQRCST | 4008 | 10.084 | 0.762 | 0.000 | 0.000 | 0.000 | 0.000 |
| KTINGSGSPHSKAQFCLT | 4009 | 10.065 | 3.371 | 0.000 | 0.000 | 0.000 | 0.000 |
| KTINGSGSPHGRYQNQQT | 4010 | 10.028 | 0.778 | 0.000 | 0.000 | 0.000 | 0.000 |

Table 25 provides the peptide sequences of 43 matured capsid variants having a raw virus count greater than 10, a CV of less than 1 for the heart samples isolated, and that also demonstrated a 4-fold or greater fold-increase in expression in the heart relative to the AAV9 control. A number of the matured variants shown in Table 25 also demonstrated increased expression in other tissues isolated from the NHPs, including the brain, muscle, and/or liver, and are therefore pan-tropic.

TABLE 25
NGS fold-enrichment of TTM-001 and TTM-002 matured AAV capsid variants in the
heart of NHPs

| | | Fold Enrichment relative to AAV9 | | | | | | |
|---|---|---|---|---|---|---|---|---|
| | SEQ | | | | | Liver | Liver | |
| | ID | Heart | Brain | DRG | Muscle | RNA | DNA | Brain |
| Sequence | NO: | (NHP) | (NHP) | (NHP) | (NHP) | (NHP) | (NHP) | (Mouse) |
| KTITGHDSPHSKAQNQQT | 4052 | 34.375 | 230.437 | 4.338 | 1.378 | 19.165 | 5.672 | 0.000 |
| KTINGSGSPHKSGQYQQT | 4053 | 33.208 | 851.414 | 8.704 | 17.754 | 17.342 | 9.915 | 12.911 |
| KTINGSGSPHKSGQDQQT | 4054 | 31.166 | 218.057 | 34.358 | 33.372 | 27.081 | 8.836 | 24.849 |
| KTINGSGSPHKSGQIQQT | 4055 | 27.293 | 201.467 | 48.033 | 12.706 | 17.874 | 13.192 | 10.912 |
| KTINGSGSPHKSGRNQQT | 4056 | 27.283 | 313.826 | 8.723 | 36.593 | 15.252 | 12.352 | 21.595 |
| KTINGSGSPHKSGKNQQT | 4057 | 25.992 | 230.621 | 6.343 | 97.671 | 15.369 | 7.226 | 31.282 |
| KTIYGHDSPHSKAQNQQT | 4058 | 25.673 | 269.879 | 3.694 | 8.391 | 11.895 | 6.197 | 0.000 |
| KTINGSGSPHKSGQNQQS | 4059 | 24.783 | 244.030 | 16.675 | 26.058 | 18.059 | 9.809 | 29.751 |
| KTINGSGSPHKSGQNLQT | 4060 | 24.464 | 392.519 | 15.629 | 0.371 | 29.977 | 18.332 | 30.446 |
| KAINGHDSPHSKAQNQQT | 4061 | 22.460 | 640.466 | 7.358 | 9.986 | 9.358 | 8.490 | 0.000 |
| KTVNGHDSPHSKAQNQQT | 4062 | 21.066 | 614.034 | 3.392 | 30.908 | 21.560 | 11.933 | 121.235 |
| KTIKGHDSPHSKAQNQQT | 4063 | 20.803 | 213.564 | 24.646 | 12.361 | 15.379 | 6.551 | 13.319 |
| KSINGHDSPHSKAQNQQT | 4064 | 20.698 | 246.819 | 7.592 | 28.235 | 11.773 | 6.888 | 280.630 |
| KTINGSGSPHKSGQTQQT | 4065 | 19.925 | 466.459 | 55.454 | 15.485 | 15.473 | 6.446 | 15.179 |
| KTINGSGSPHKSGHNQQT | 4066 | 19.548 | 287.922 | 12.159 | 20.851 | 17.821 | 10.084 | 21.011 |
| KTENGHDSPHSKAQNQQT | 4067 | 19.301 | 239.922 | 9.109 | 17.215 | 12.193 | 6.413 | 30.747 |
| KTINGSGSPHKSGLNQQT | 4068 | 19.136 | 319.093 | 3.083 | 4.096 | 14.009 | 7.446 | 9.340 |
| KTINGHDSPHSKALNQQT | 4069 | 18.542 | 605.641 | 13.375 | 1.902 | 12.621 | 7.054 | 51.283 |
| KTINGSGSPHKSGQNQLT | 4070 | 18.454 | 317.452 | 33.967 | 28.952 | 18.533 | 8.992 | 36.272 |
| KTLNGHDSPHSKAQNQQT | 4071 | 18.236 | 195.734 | 19.341 | 9.266 | 25.732 | 13.333 | 0.000 |
| KTINGSGSPHKSGQNQHT | 4072 | 14.269 | 313.837 | 7.125 | 39.273 | 29.714 | 7.797 | 25.119 |
| KTIDGHDSPHSKAQNQQT | 4073 | 13.836 | 242.100 | 1.731 | 12.555 | 17.223 | 7.439 | 0.000 |
| KTNNGHDSPHSKAQNQQT | 4074 | 12.872 | 134.488 | 0.504 | 3.877 | 17.044 | 5.982 | 22.358 |
| KTINGSGSPHKSGQKQQT | 4075 | 12.357 | 323.373 | 10.936 | 1172.3 | 12.604 | 7.970 | 48.699 |
| KTINGSGSPHKSGQNRQT | 4076 | 11.563 | 145.363 | 36.865 | 3.855 | 11.403 | 7.667 | 16.860 |
| KTINGSGSPHKSGQNQQN | 4077 | 11.507 | 156.385 | 582.38 | 8.559 | 9.273 | 7.668 | 18.138 |
| KTINGSGSPHKSGQNQQA | 4078 | 11.313 | 135.164 | 12.425 | 12.699 | 9.714 | 6.077 | 17.265 |
| KTINGHDSPHSKAHNQQT | 4079 | 10.024 | 236.106 | 19.495 | 5.258 | 2.406 | 3.316 | 45.691 |
| KTINGHDSPHSKAQNQQT | 4080 | 8.954 | 186.839 | 9.457 | 5.507 | 5.929 | 3.651 | 31.453 |
| KTINGSGSPHKSGQNQQP | 4081 | 8.744 | 261.947 | 43.435 | 10.217 | 6.468 | 4.265 | 19.828 |
| KTINGHDSPHCKAQNQQT | 4082 | 8.417 | 15.165 | 0.887 | 2.368 | 3.328 | 0.771 | 148.172 |
| KTINGHDSPHSKAQNQQS | 4083 | 5.678 | 603.027 | 7.280 | 0.670 | 4.301 | 4.307 | 65.271 |
| KTINGSGSPHKSGQNQQT | 4084 | 5.586 | 115.994 | 28.397 | 4.326 | 5.307 | 3.569 | 24.908 |
| KTINGHDSPDKSGQNQQT | 4085 | 5.569 | 30.854 | 4.934 | 1.112 | 0.671 | 0.781 | 14.499 |
| KPINGHDSPHKSGQNHQS | 4086 | 5.203 | 36.266 | 0.000 | 0.258 | 4.478 | 0.521 | 28.786 |
| KTSNGSGSPHKSGQNQQT | 4087 | 4.746 | 197.282 | 4.177 | 4.466 | 3.972 | 7.425 | 75.623 |
| KTVNGSGSPHKSGQNQQT | 4088 | 4.610 | 200.076 | 2.739 | 2.873 | 2.725 | 3.478 | 43.548 |
| KTINGHDSTHKSGHNQQT | 4089 | 4.369 | 27.630 | 2.883 | 1.302 | 0.421 | 0.176 | 12.973 |
| KTINGHDSPHSKAQNQQN | 4090 | 4.271 | 319.610 | 1.163 | 5.173 | 3.406 | 4.995 | 50.220 |
| KTIYGSGSPHKSGQNQQT | 4091 | 4.140 | 110.329 | 2.603 | 2.545 | 4.488 | 4.110 | 29.293 |
| KTINGLDSQHKSGQNQQT | 4092 | 4.055 | 12.958 | 3.240 | 3.205 | 0.645 | 0.296 | 5.608 |

Table 26 provides the peptide sequences of 14 matured capsid variants having a raw virus count greater than 10, a CV of less than 1 for the muscle samples isolated (e.g., quadriceps), and that also demonstrated a 4-fold or greater fold-increase inexpression in the muscle relative to the AAV9 control. A number of the matured variants shown in Table 26 also demonstrated increased expression in other tissues isolated from the NHPs, including the brain, heart, and/or liver, and are therefore pan-tropic.

TABLE 26
NGS fold-enrichment of TTM-001 and TTM-002 matured AAV capsid variants in the
muscle (e.g., quadriceps) of NHPs

| | | Fold Enrichment relative to AAV9 | | | | | | |
|---|---|---|---|---|---|---|---|---|
| | SEQ | | | | | Liver | Liver | |
| | ID | Muscle | Brain | DRG | Heart | RNA | DNA | Brain |
| Sequence | NO: | (NHP) | (NHP) | (NHP) | (NHP) | (NHP) | (NHP) | (Mouse) |
| KTINGSGSPHKSGRNQQT | 4056 | 36.593 | 313.826 | 8.723 | 27.283 | 15.252 | 12.352 | 21.595 |
| KTIIGHDSPHSKAQNQQT | 4095 | 27.271 | 341.528 | 5.423 | 26.154 | 18.305 | 6.293 | 0.000 |
| KTIYGHDSPHSKAQNQQT | 4058 | 8.391 | 269.879 | 3.694 | 25.673 | 11.895 | 6.197 | 0.000 |
| KTINGSGSPHKSGQNQQS | 4059 | 26.058 | 244.030 | 16.675 | 24.783 | 18.059 | 9.809 | 29.751 |
| KTVNGHDSPHSKAQNQQT | 4062 | 30.908 | 614.034 | 3.392 | 21.066 | 21.560 | 11.933 | 121.235 |
| KTIKGHDSPHSKAQNQQT | 4063 | 12.361 | 213.564 | 24.646 | 20.803 | 15.379 | 6.551 | 13.319 |
| KSINGHDSPHSKAQNQQT | 4064 | 28.235 | 246.819 | 7.592 | 20.698 | 11.773 | 6.888 | 280.630 |
| KTINGSGSPHKSGHNQQT | 4066 | 20.851 | 287.922 | 12.159 | 19.548 | 17.821 | 10.084 | 21.011 |
| KTENGHDSPHSKAQNQQT | 4067 | 17.215 | 239.922 | 9.109 | 19.301 | 12.193 | 6.413 | 30.747 |
| KTSNGHDSPHSKAQNQQT | 4096 | 18.580 | 507.189 | 7.777 | 17.770 | 21.537 | 8.789 | 70.219 |
| KTINGHDSPHSKAQNQQT | 4080 | 5.507 | 186.839 | 9.457 | 8.954 | 5.929 | 3.651 | 31.453 |
| KTINGSGSPHKSGQNQQT | 4084 | 4.326 | 115.994 | 28.397 | 5.586 | 5.307 | 3.569 | 24.908 |
| KTINGHDSPHSKAQNQQN | 4090 | 5.173 | 319.610 | 1.163 | 4.271 | 3.406 | 4.995 | 50.220 |
| KTINGSGSPHSKAQNRRR | 4097 | 4.237 | 8.348 | 0.291 | 0.636 | 1.597 | 5.396 | 158.853 |

Additional variants were identified following generation and screening in NHPs that had the following properties. TTM-001 and TTM-002 capsid variants comprising the amino acid sequence of SEQ ID NOs: 4253, 4281, 4290-4295, 4304, 4305, 4320, 4328-4335, 4337-4340,4353,4355, 4369, 4387, 4421, 4424-4428, 4430, 4432, 4433, 4435, 4436-4449, 4452, 4455, 4476, 4483, or 4484 had a raw virus count 10 or greater, a CV of less than 1 for the brain samples isolated from the NHPs, demonstrated a 50-fold or greater increase in expression in the brain of mice and NHPs relative to AAV9, and demonstrated 2-fold or less expression in the liver and DRG of NHPs relative to AAV9. TTM-001 and TTM-002 capsid variants comprising the amino acid sequence of SEQ ID NOs: 4098-4105, 4254-4280, 4282-4289, 4296-4303, 4306-4327, 4336, 4341-4352, 4354, 4356-4420, 4422, 4423, 4425, 4429, 4431, 4434, 4444, 4450, 4451, 4453,4454,4456-4475,44774482, or 4485 had a CV of less than 1 in across the brain samples isolated from the NHPs and demonstrated a 100-fold or greater increase in expression in the brain of NHPs relative to AAV9, TTM-001 and TTM-002 capsid variants comprising the amino acid sequence of SEQ ID NOs: 4102 and 41064252 had normalized virus counts of greater than or equal to 0.01, a CV of less than 1 across the liver RNA samples isolated from the NHPs, and demonstrated a 20-fold or greater increase in expression in the liver of NHPs relative to AAV9. TM-001 and TTM-002 capsid variants comprising the amino acid sequence of SEQ ID NO: 4105 had a raw vims count 9.9 or greater, a CV of less than 1 across the muscle samples isolated from the NHPs, and 5-fold or greater increase in expression in the muscle of the NHPs relative to AAV9. TM-001 and TTM-002 capsid variants comprising the amino acid sequence of SEQ ID NO: 4105 also had a raw virus count 9.9 or greater, a CV of less than 1 across the samples isolated from the heart of the NHPs, and 5-fold or greater increase in expression in the heart of the NHPs relative to AAV9.

These data demonstrate that following two maturation approaches, matured TM-001 and TTM-002 capsid variants (AAV9 capsid variants) with loop IV modifications were generated with significantly enhanced CNS tropism over wild-type AAV9 controls in both NHPs and mice, while also exhibiting dc-targeting in peripheral tissues (e.g., the liver and DRG). These resulting matured variants therefore demonstrated cross-species CNS tropism in both NHPs and mice. Matured TTM-001 and TTM-002 capsid variants with liver-specific tropism were also generated with at least 10 times the expression compared to wild-type AAV9 in the liver of NHPs. Several matured variants were also generated with increased expression in the heart and skeletal muscle (e.g., quadriceps) relative to wild-type AAV9 in NHPs.

### Example 5. Evaluation of TTM-001 and TTM-002 AAV Capsid Variants in Diverse Primate Species

This Example evaluates the tropism and cross-species compatibility of the TTM-001 (SEQ ID NO: 981 (amino acid) and 983 (DNA), comprising SEQ ID NO: 941) and TTM-002 (SEQ ID NO: 982 (amino acid) and 984 (DNA), comprising SEQ ID NO: 2) capsid variants in two diverse primate species, marmosets (Callithrix jacchus) and African green monkeys (Chlorocebus sabaeus), as compared to their tropism in Cynomolgus macaques (Macaca fascicularis) provided in Example 1. The cross-species compatibility and tropism of an AAV9 capsid variant comprising the amino acid sequence of SPHKYG (SEQ ID NO: 966) was also investigated in this example. The amino acid and DNA sequences of TTM-001 and TTM-002 are provided, e.g., in Tables 4 and 5, respectively.

To investigate tropism in African green monkeys. AAV particles comprising the TTM-001 capsid variant, the TTM-002 capsid variant, an AAV9 capsid variant comprising SEQ ID NO: 966, or an AAV9 control under the control of a synapsin promoter, were intravenously injected into NHPs (n=2, 3-12 years of age) at a dose of 2E13 vg/kg. After 14-days in life, the brains and tissues (liver, DRG, quadriceps, and heart) of the NHPs were collected and RNA was extracted. Following RNA recovery and RT-PCR amplification, a systematic NGS enrichment analysis was performed to calculate the fold enrichment ratio relative to the AAV9 wild-type control.

To investigate tropism in marmoset monkeys. AAV particles comprising the TTM-00 capsid variant, the TTM-002 capsid variant, an AAV9 capsid variant comprising SEQ ID NO: 966, or an AAV9 control, were intravenously injected into NHPs (n=2, >10 months of age) at a dose of 2E13 vg/kg (8.75E12 vg/mL), After 28-days in life, the brains and tissues (liver quadriceps, and heart) of the NHPs were collected and RNA was extracted. Following RNA recovery and RT-PCR amplification, a systematic NGS enrichment analysis was performed to calculate the fold enrichment ratio relative to the AAV9 wild-type control.

As provided in Table 27 (African green monkeys) and Table 28 (marmosets), both the TTM-001 and TTM-002 capsid variants demonstrated increased CNS tropism in diverse primate species. The TTM-001 capsid variant demonstrated a 73.6-fold increase in expression relative to AAV9 in the brain of Cynomolgus macaques (Table 16. Example 1), a 43.5-fold increase in expression relative to AAV9 in the brain of African green monkeys, and a 703.3-fold increase in expression relative to AAV9 in the brain of marmosets. The TTM-02 capsid variant demonstrated a 62.6-fold increase in expression relative to AAV9 in the brain of Cynomolgus macaques (Table 16), a 13.8-fold increase in expression relative to AAV9 in the brain of African green monkeys, and a 366.6-fold increase in expression relative to AAV9 in the brain of marmosets. Both TTM-001 and TTM-002 led to a significant increase in expression relative to AAV9 in the heart of both African green monkeys and marmosets (Table 27 and Table 28). The AAV9 capsid variant comprising SEQ ID NO: 966 also demonstrated in increase in expression relative to AAV9 in the brain and heart of both African green monkeys and marmosets. Furthermore. TTM-001, TTM-002, and the AAV9 capsid variant comprising SEQ ID NO: 966, also all led to increased expression in the brain of both BALB/c and C57B1/6 mice (Table 18, Example 1), demonstrating an average fold change in expression relative to AAV9 across both species of mice of 63.1, 66.8, and 126.97, respectively.

TABLE 27
NGS-fold enrichment of TTM-001 (comprises SEQ ID NO: 941), TTM-002 (comprises
SEQ ID NO: 2), and an AAV9 capsid variant comprising SEQ ID NO: 966 in
African green monkeys

| | SEQ ID | Fold Enrichment relative to AAV9 | | | | | |
|---|---|---|---|---|---|---|---|
| Sequence | NO: | Brain | DRG | Heart | Liver DNA | Liver RNA | Muscle |
| SPHSKA | 941 | 43.525 | 1.010 | 184.789 | 0.242 | 1.547 | 1.715 |
| HDSPHK | 2 | 13.779 | 0.678 | 35.991 | 0.084 | 0.087 | 0.144 |
| SPHKYG | 966 | 9.805 | 0.071 | 44.865 | 0.085 | 0.136 | 0.234 |

TABLE 28
NGS-fold enrichment of TTM-001 (comprises SEQ ID NO: 941), TTM-002
(comprises SEQ ID NO: 2), and an AAV9 capsid variant comprising SEQ
ID NO: 966 in marmosets

| | SEQ ID | Fold Enrichment relative to AAV9 | | | | |
|---|---|---|---|---|---|---|
| Sequence | NO: | Brain | Heart | Liver DNA | Liver RNA | Muscle |
| SPHSKA | 941 | 703.610 | 48.979 | 0.268 | 0.779 | 0.425 |
| HDSPHK | 2 | 366.625 | 18.572 | 0.075 | 0.276 | 0.229 |
| SPHKYG | 966 | 150.209 | 17.232 | 0.045 | 0.014 | 0.146 |

Taken together, these data demonstrate that the AAV9 capsid variants of TTM-00 I and TTM-002 demonstrated increased CNS tropism relative to the AAV9 control in the CNS across three diverse primate species and two species of mice, providing evidence of strong cross-species capacity. The AAV9 capsid variant comprising the amino acid sequence of SEQ ID NO: 966 also demonstrated strong CNS expression relative to the AAV9 control in two species of NHPs and two species of mice, also showing strong cross-species capacity.

### Example 6. Advanced Maturation of TTM-002 Capsid Variant in Mice

This Example describes additional maturation of the TTM-002 (SEQ ID NO: 982 (amino acid) and 984 (DNA), comprising SEQ ID NO: 2) capsid variant in mice. In order to mature the TTM-002 capsid variant, sets of three contiguous amino acids were randomized across the mutagenesis region in TTM-002 sequence, which spanned from position 450 to position 466, numbered according to SEQ ID NO: 982. Unlike the maturation performed in in Example 3, where the SPH motif that was observed in the AAV capsid variants that demonstrated the greatest fold-enrichment in the NHP brain relative wild-type AAV9 was not disrupted, in the maturation approach used in this Example, the SPH motif was not held constant to further explore the role of this motif in the capsid variant. The matured TTM-002 capsid variants that resulted from the maturation approach were pooled together for subsequent testing and characterization in mice.

The library of matured AAV capsid variants generated from the TTM-002 matured AAV capsid variant were intravenously injected into the tail vein of three CD-1 Outbred mice (Charles River, 6-8 weeks of age) at a dose of 1.0×10¹² VG/dose. After about 28 days in life, the brains of the mice were isolated, and RNA was extracted. Following RNA recovery and RT-PCR amplification, a systematic NGS enrichment analysis was performed to calculate the fold enrichment ratio relative to the corresponding TTM-002 non-matured control, and the peptides comprised within the variants were identified. Variants were filtered by those with a raw virus count in the sample above 10 and a coefficient of variance (CV) that was greater than 1 (identifies the peptides/variants reliably detected in the majority of the samples isolated from the three mice).

Following the advanced maturation screen and filtering of the variants, 1302 variants demonstrated an increase in expression relative to the non-matured TTM4002 capsid variant in the brain of the outbred mice. Of the 1302 variants with improved tropism relative to the non-matured TTM-002, 1283 comprised the SPH motif in the same position as the non-matured TTM-002 capsid variant (e.g., immediately subsequent to position 455, relative to a reference sequence numbered according to the amino acid sequence of SEQ ID NO: 138 or 982), Mutations in the region of the SPH motif present in the non-matured TTM-002 capsid variant only consistently appear in those variants with a fold change of 0.2 or 0.1 or lower relative to the non-matured TM-002 control in the brain of the mice. This indicates that the SPH motif may be important to the increased brain tropism that observed for the TTM-02 capsid variant. In instances when the SPH motif was disrupted, the fold change of the matured variants of TTM-002 decreased considerably in relation to the non-matured TTM-002 variant which comprised the SPH motif.

### Example 7. Tropism of TTM-002 AAV Capsid Variant

This Example further investigates the tropism and CNS cells transduced by the TTM-002 capsid variant (SEQ ID NO: 982 (amino acid) and 984 (DNA), comprising SEQ ID NO: 2), as outlined in Table 3 above. The amino acid and DNA sequences of TTM402 are provided, e.g., in Tables 4 and 5, respectively.

AAV particles were generated with the TTM-002 capsid variant encapsulating a GFP transgene (AAV_TTM-002.GFP) or a payload driven by a heterologous CBA constitutive promoter (AAV_TTM-002.Payload).

Two tandem single cell RNA sequencing runs (scRNA-Seq) of mouse cells derived from the midbrain area were performed. In the first run, cells were pooled from two mice at day 28 post treatment with AAV_TrM-002.Payload particles. In the second run we treated with AAV_TTM-002.GFP particles, in the same manner but without xenografts. Orthotopic xenografts of MDA-MB-361-Luc #1 high passage cells grown as tumorspheres (in tumorsphere media: Sigma #C-28070) were injected (250,000 cells/2 μL/mouse) intracranially into 2-month old female SCID CB17 (Mutation: Icr-Prkdcscid/IcrIcoCrl) congenic immunodeficient mice (Charles River Laboratories). The injections were 2.5 mm (lateral). −1 mm (posterior) with respect to bregma, lowered −3 mm ventral and raised+0.5 mm dorsal to a final −2.5 mm ventral position. Two days later, dilutions of the AAV_TTM-002.Payload particles (run 1), or in the case without xenografts, dilutions of AAV_TIM-002.GFP particles (run 2) were prepared. IV injections of 100 μL (2.5eI1 VG/animal) of the AAV_TTM-002.payload particles or AAV_TTM-002.GFP particles were administered through the tail veins of mice (n=5 mice per groups), At 7 days post-injection, mice from run 1 were imaged in an AmiHTX (Spectral Imager) for bioluminescence of the human tumor cells due to expression of luciferase in response to intraperitoneal luciferin injections.

At 28 days post-injection with the AAV_TTM-002.payload particles or AAV_TTM-002.GFP particles, two mice from each run were necropsied, brain samples were isolated, and the midbrain was dissected and isolated. The midbrain samples were then exposed to a cold protease inhibitor (Creative Biomart #NATE-0633) and were dissociated at 6 degrees centigrade. For the samples collected from the mice of run 1 (AAV_TTM-002.Payload particles), myelin depletion was performed (Miltenyi. #130-096-731), cells were filtered through a 40 μM mesh to filter out neurons) and loaded on a 10× chromium G chip, scRNA-Seq was performed (10× Genomics) and samples were sequenced on a NextGen500 Sequencing machine (Illumina). For the samples collected from run 2 (AAV_TTM-002.GFP particles and no xenografts), the cells were not myelin depleted or filtered through 40tM mesh to include neurons. The cells isolated after run 2 were FACS sorted for GFP+/7AAD-(live GFP+ cells). The resultant cells were loaded on a 10× chromium G chip and the scRNA-Seq was run and processed (10× Genomics).

For run 1, the scRNA-Seq data was filtered to include cells with only greater than 1000 genes per cell and less than 5000, and less than 20 percent mitochondrial gene expression. For run 2, the scRNA-Seq data was filtered to include cells with only greater than 200 genes per cell and less than 5000, and less than 20 percent mitochondrial gene expression. The data were normalized, scaled, and integrated into one combined dataset. Clusters were generated with a resolution of 0.3 and each cluster identity was determined using a panel of cell type specific genes (e.g., as described in Brown et al., 2021. “Deep Parallel Characterization of AAV Tropism and AAV-Mediated Transcriptional Changes via Single-Cell RNA Sequencing”. Front. Immnol. 12:730825; the contents of which are hereby incorporated by reference in its entirety). The percentage of GFP sorted cells per cluster was calculated as was the percentage of payload expressing genes per cluster as parallel measures of TTM-02 transduction.

For payload expressing cells, endothelial cells had the highest proportion of payload positive cells, followed by astrocytes (Table 29). For GFP+ sorted cells, endothelial cells had the highest proportion of GFP positive cells, and astrocytes were the third highest cell type when sorting by proportion of cells expressing GFP (Table 29). These data indicate TTM-002 transduction exhibits an endothelial and astrocytic tropism. Furthermore, the astrocytic cluster had the second highest level of expression of Olig2 (oligodendrocytes demonstrated the greatest Olig2 expression). IHC staining was performed on brain samples isolated from AAV_TTM-002.GFP infected mice and demonstrated that GFP co-localized with some but not all Olig2+ cells. No co-staining was observed with mylein basic protein (MBP), a marker of oligodendrocytes. Co-staining with GFP was also not observed in NeuN positive cells (neurons), GFAP positive cells (astrocytes), and Iba1 positive cells (microglia). GFP staining was observed throughout the sagittal section of the mouse brain, which was demonstrative of increased staining in the midbrain. The GFP expressing cells observed did not have a bipolar morphology like oligodendrocyte progenitor (OPC) cells and therefore, together with the scRNA-Seq data, these results indicated that at day 28 post AAV treatment, Olig2+ astrocytes in the midbrain are being transduced by AAV particles comprising a TTM-002 capsid, in a cell type specific tropism.

TABLE 29
Quantification of payload positive cells and GFP positive cells

| | % Payload | | % GFP |
|---|---|---|---|
| Cluster | Cells/ | Cluster | Cells/ |
| Identity | Cluster | Identity | Cluster |
| Endothelial-2 | 6.58 | Endothelial-2 | 6.58 |
| Astrocyte | 4.50 | Endothelial-1 | 3.45 |
| Pericytes | 4.23 | Vascular and | 2.38 |
| | | leptomeningeal | |
| | | Cells (VLM) | |
| Mature Oligos | 3.85 | Astrocyte | 2.37 |
| Endothelial-1 | 3.09 | Vascular smooth | 1.03 |
| | | muscle cells (VSC) | |
| Committed Oligos | 1.90 | Pericytes | 0.77 |
| Vascular smooth | 1.72 | Microglia | 0.00 |
| muscle cells (VSC) | | | |
| Microglia | 0.40 | Committed Oligos | 0.00 |
| Macrophages | 0.00 | Macrophages | 0.00 |
| Vascular and | 0.00 | Oligodendrocytes | 0.00 |
| leptomeningeal | | | |
| cells (VLM) | | | |
| Oligodendrocytes | 0.00 | Committed Oligos-2 | 0.00 |
| Committed Oligos-2 | 0.00 | Mature Oligos | 0.00 |

Example 8. Individual Capsid Characterization of TTM-001, TTM-002, TTM-003, TTM-006, and TTM-027 in NHPs

This example describes the transduction level, tropism, ability to cross the blood brain barrier, and overall spatial distribution in the central nervous system (CNS) and peripheral tissues of the AAV capsid variants TTM-002 (SEQ ID NO: 982 (amino acid) and SEQ ID NO: 984 (DNA), comprising SEQ ID NO: 2). TTM-001 (SEQ ID NO: 981 (amino acid) and SEQ ID NO: 983 (DNA), comprising SEQ ID NO: 941); TTM-403 (SEQ ID NO: 36 (amino acid) and SEQ ID NO: 12 (DNA), comprising SEQ ID NO: 3589). TTM-006 (SEQ ID NO: 39 (amino acid) and SEQ ID NO: 15 (DNA), comprising SEQ ID NO: 3241), and/or TM-027 (SEQ ID NO: 4 (amino acid) and SEQ ID NO: 5 (DNA), comprising SEQ ID NO: 3272), relative to AAV9 following intravenous administration in African green monkeys (Chlorocebus sabaeus), marmosets (Callithrix jacchus), and/or cynomolgus macaques (Macaca fascicularis).

A. Evaluation of TTM-002 in African Green a Monkeys (Chlorocebus sabaeus)

AAV particles were generated with the TTM-002 capsid variant or the AAV9 capsid control which comprised a self-complementary viral genome encoding an histone H2b protein with an HA tag driven by a ubiquitous CBA promoter. The AAV particles comprising the TTM-002 capsid variant or the AAV9 capsid control were administered to the NHPs (n=2) intravenously at a dose of 1e12 VG/kg or 1e13 VG/kg. The in-life period was 28 days and then various CNS and peripheral tissues were collected for measuring transgene mRNA (expression) by RT-qPCR and viral DNA (biodistribution) by ddPCR.

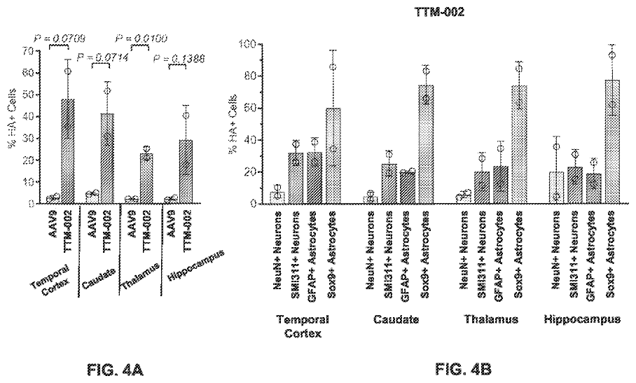

As shown in Table 30, the TTM4-002 capsid variant resulted in increased brain biodistribution in all brain regions investigated as compared to AAV9 at both doses tested. The TTM-002 capsid variant also led to increased transgene expression in the brain relative to AAV9 at both doses tested (Table 31). In the spinal cord, the TTM-002 capsid variant distributed to the cervical spinal cord and the spinal cord ventral horn at a higher level relative to AAV9 (Table 30) and it mediated higher transgene expression than AAV9 in both the full spinal cord and the ventral horn (Table 31). When administered at a dose of 1e13 VG/kg. TTM-002 delivered 1-2 viral genomes (VGs) per cell on average across multiple brain areas, outperforming AAV9 by 4- to 24-fold, and was capable of expressing 16- to 186-fold more transgene RNA (Table 30 and Table 31). The TTM-002 capsid variant exhibited lower biodistribution (Table 30) and transgene expression (Table 31) in the DRG relative to AAV9, indicating that TTM-002 capsid variant was detargeted in the DRG relative to AAV9, Similar expression and distribution were observed by immunohistochemistry performed on these CNS tissues. High-throughput analysis of immunohistochemistry stainings indicated that TTM-002 was capable of targeting upwards of 50% of cells in the brain (FIG. 4A), including both astrocytes and neurons (FIG. 4B). In contrast with the tropism in mice as provided in Example 9. TTM-002 demonstrated a bias towards Sox9 (+) astrocytes over neurons, labeled with either NeuN or SM1311.

Distribution and transgene expression was also measured in the peripheral tissues of the liver, heart, and quadriceps. In the liver, TTM-002 capsid variant exhibited lower biodistribution (Table 30) and transgene expression (Table 31) relative to AAV9, indicating that TTM-002 capsid variant was detargeted in the liver relative to AAV9. In the heart, the TTM-002 capsid variant exhibited comparable levels of biodistribution relative to AAV9 (Table 30), but increased transgene expression relative to AAV9 (Table 31). In the quadriceps, TM-002 capsid variant exhibited lower biodistribution (Table 30) and lower transgene expression (Table 31), relative to AAV9, Similar expression and distribution were observed by immunohistochemistry performed on these peripheral tissues.

TABLE 30
Quantification of viral genome copies per diploid genome (biodistribution) by ddPCR
following intravenous administration of AAV particles comprising a TTM-002 capsid

| | 1e12 VG/kg | | | 1e13 VG/kg | | |
|---|---|---|---|---|---|---|
| | AAV9 | TTM-002 | | AAV9 | TTM-002 | |
| | (VG copies/ | (VG copies/ | TTM-002 | (VG copies/ | (VG copies/ | TTM-002 |
| | diploid | diploid | relative | diploid | diploid | relative |
| Tissue | genome) | genome) | to AAV9 | genome) | genome) | to AAV9 |
| Putamen | 0.03 | 0.37 | 12.3 | 0.26 | 2.4 | 9.2 |
| Caudate | 0.02 | 0.58 | 29 | 0.14 | 2.1 | 14.7 |
| Thalamus | 0.06 | 0.21 | 3.5 | 0.25 | 1.0 | 4 |
| Hippocampus | 0.03 | 0.29 | 9.7 | 0.16 | 1.56 | 9.8 |
| Substantia Nigra | 0.05 | 0.34 | 6.8 | 0.37 | 1.38 | 3.7 |
| Motor Cortex | 0.03 | 0.56 | 19 | 0.27 | 2.4 | 8.9 |
| Frontal Cortex | 0.04 | 0.67 | 17 | 0.20 | 3.6 | 18 |
| Temporal Cortex | 0.03 | 0.31 | 10 | 0.11 | 2.67 | 24 |
| Cerebral Cortex | 0.008 | 0.08 | 10 | 0.03 | 0.16 | 5.3 |
| Dentate Nucleus | 0.06 | 0.10 | 1.7 | 0.32 | 3.21 | 10 |
| Cervical Spinal Cord | 0.03 | 0.12 | 4 | 0.19 | 0.91 | 4.8 |
| Thoracic Spinal Cord | 0.04 | 0.03 | 0.75 | 0.36 | 0.38 | 1.1 |
| Lumbar Spinal Cord | 0.04 | 0.03 | 0.75 | 0.29 | 0.37 | 1.3 |
| C5 Ventral Horn | 0.04 | 0.25 | 6.3 | 0.29 | 2.2 | 7.6 |
| L5 Ventral Horn | 0.06 | 0.28 | 4.7 | 0.31 | 1.9 | 6.1 |
| Cervical DRG | 0.07 | 0.01 | −7 | 0.81 | 0.36 | −2.3 |
| Thoracic DRG | 0.06 | 0.01 | −6 | 1.31 | 0.43 | −3 |
| Lumbar DRG | 0.07 | 0.01 | −7 | 1.31 | 0.57 | −2.3 |
| Liver | 9.5 | 1.2 | −7.9 | 127 | 7.7 | −16.5 |
| Heart | 0.6 | 0.7 | 1.2 | 5.4 | 5.4 | 1 |
| Quadriceps | 0.2 | 0.06 | −3.3 | 1.7 | 0.6 | −2.8 |

TABLE 31
Quantification of transgene mRNA by RT-qPCR following intravenous
administration of AAV particles comprising a TTM-002 capsid

| | 1e12 VG/kg | | | 1e13 VG/kg | | |
|---|---|---|---|---|---|---|
| | AAV9 | TTM-002 | | AAV9 | TTM-002 | |
| | (transgene | (transgene | | (transgene | (transgene | |
| | mRNA fold | mRNA fold | | mRNA fold | mRNA fold | |
| | over | over | TTM-002 | over | over | TTM-002 |
| | housekeeping | housekeeping | relative | housekeeping | housekeeping | relative |
| Tissue | gene) (2^{-dCT}) | gene) (2^{-dCT}) | to AAV9 | gene) (2^{-dCT}) | gene) (2^{-dCT}) | to AAV9 |
| Putamen | 0.02 | 0.3 | 15 | 0.09 | 4.22 | 47 |
| Caudate | 0.02 | 0.8 | 40 | 0.11 | 4.29 | 39 |
| Thalamus | 0.04 | 0.4 | 10 | 0.4 | 5.8 | 14.5 |
| Hippocampus | 0.02 | 0.4 | 20 | 0.1 | 4.3 | 43 |
| Substantia Nigra | 0.1 | 1.2 | 12 | 0.3 | 11.6 | 39 |
| Motor Cortex | 0.08 | 5.00 | 63 | 0.36 | 21.8 | 61 |
| Frontal Cortex | 0.04 | 3.1 | 78 | 0.3 | 27.7 | 92 |
| Temporal Cortex | 0.02 | 0.8 | 40 | 0.1 | 26.9 | 27 |
| Cerebral Cortex | 0.04 | 1.1 | 28 | 0.2 | 17.4 | 87 |
| Dentate Nucleus | 0.3 | 0.9 | 3 | 1.8 | 42.0 | 23 |
| Cervical Spinal Cord | 0.2 | 2.0 | 10 | 0.8 | 20.2 | 25 |
| Thoracic Spinal Cord | 0.13 | 0.25 | 1.9 | 0.7 | 4.8 | 6.9 |
| Lumbar Spinal Cord | 0.4 | 0.5 | 1.3 | 2.2 | 9.2 | 4.2 |
| CS Ventral Horn | 0.2 | 1.4 | 7 | 1.7 | 33 | 19 |
| L5 Ventral Horn | 1.1 | 3.4 | 3.1 | 12.4 | 102 | 8.2 |
| Cervical DRG | 3.6 | 1.2 | −3 | 63.1 | 15.9 | −4 |
| Thoracic DRG | 1.8 | 1.3 | −1.4 | 43.9 | 15.7 | −2.8 |
| Lumbar DRG | 1.9 | 1.0 | −1.9 | 34.9 | 27.6 | −1.3 |
| Liver | 0.88 | 0.25 | −3.5 | 2.2 | 0.97 | −2.3 |
| Heart | 8.7 | 42 | 4.8 | 110 | 363 | 3.3 |
| Quadriceps | 9.7 | 1.1 | −8.3 | 59 | 21 | −2.8 |

Taken together, these data demonstrate that TTM-002 is an enhanced CNS tropic capsid in NHPs (African green monkeys) that can infect non-neuronal cells. TTM-002 was also detargeted in the DRG and liver relative to AAV9, but showed increased transgene expression in the heart relative to AAV9. Additionally, the TTM-002 capsid variant was able to successfully penetrate the blood brain barrier following intravenous injection.

B. Evaluation of TM-001 and T7M-002 in Marmosets (Callithrix jacchus)

AAV particles were generated with the TTM-002 capsid variant, the TTM-001 capsid variant, or the AAV9 capsid control, each of which comprised a self-complementary viral genome encoding a histone H2b protein with an MYC tag (TTM-002 capsid variant), His tag (TTM-001 capsid variant), or HA tag (AAV9 control capsid) driven by a ubiquitous CAG promoter. The AAV particles comprising the TTM-02 capsid variant, the TTM-001 capsid variant, or the AAV9 capsid control were administered to the marmosets (Callithrix jacchus) (n=3) intravenously in a single solution, at the doses indicated in Table 51. The in-life period was 28 days and then various CNS and peripheral tissues were collected for measuring transgene mRNA (expression) by RT-qPCR, protein expression by IHC, and viral DNA (biodistribution) by ddPCR. Data were then normalized to the dose of each viral vector in the dosing solution.

TABLE 51
Titer of the AAV particles comprising the various
capsids in solution dosed in marmosets

| | | Ratio of Capsid |
|---|---|---|
| Capsid Variant | Actual Titer Dosed | Variant to AAV9 |
| TTM-001 | 1.44 × 10¹¹ vg/mL | 0.34 |
| AAV9 | 4.00 × 10¹¹ vg/mL | 1.0 |
| TTM-002 | 4.17 × 10¹¹ vg/mL | 1.0 |

As shown in Table 52, both the TTM-00I and TTM-002 capsid variants demonstrated increased biodistribution in the caudate and motor cortex in the brain of the marmosets relative to the AAV9 control. The TTM-001 and TTM-002 capsid variants also led to increased transgene expression (Table 53) in the caudate and motor cortex in the brain of the marmosets. In fact, biodistribution and transgene expression were increased over 100400 fold for both TTM-00I and TTM-002 in the brain relative to AAV9, TTM-002 delivered upwards of 280-fold more viral genomes and expressed 500-fold higher transgene RNA levels than AAV9 (Tables 52 and 53), Similar expression and distribution was observed by immunohistochemistry. More specifically, staining for TTM-N001 and TTM-002 was detected in the mid-brain, caudate, putamen, thalamus, and cerebellum, and this staining was increased for both capsid variants in each of these brain tissues relative to AAV9, Staining for TTM-001 and TTM-002 was also observed in the molecular and granule layer of the cerebellum.

Biodistribution and transgene expression were also measured in the peripheral tissues of the liver, heart, and quadriceps. In the liver, the TTM-002 capsid variant exhibited lower biodistribution (Table 52) and transgene expression (Table 53) relative to AAV9, indicating that the TTM-002 capsid variant was detargeted in the liver relative to AAV9 in marmosets. The TM-001 capsid variant demonstrated comparable biodistribution and transgene expression in the liver (Table 52 and Table 53) as well as comparable transgene expression in the heart and muscle (Table 53) relative to AAV9. Both TTM-001 and TTM402 led to decreased biodistribution (Table 52) relative to AAV9 in the heart and muscle, and TTM-002 also resulted in lower transgene expression in the heart and muscle relative to AAV9 (Table 53).

TABLE 52
Quantification of viral genome copies per diploid genome (biodistribution) by
ddPCR following intravenous administration of AAV particles comprising a TTM-
001 capsid or a TTM-002 capsid normalized to the actual titer of the viral vector
in the dosing solution (vg/dg = viral genome copies/diploid genome)

| | Tissue | | | | | | | | | |
|---|---|---|---|---|---|---|---|---|---|---|
| | Caudate | | Motor Cortex | | Heart | | Muscle | | Liver | |
| | | vg/dg | | vg/dg | | vg/dg | | vg/dg | | vg/dg |
| | | relative | | relative | | relative | | relative | | relative |
| Capsid | vg/dg | to AAV9 | vg/dg | to AAV9 | vg/dg | to AAV9 | vg/dg | to AAV9 | vg/dg | to AAV9 |
| TTM-001 | 1.67 | 142.70 | 2.69 | 124.06 | 0.28 | 0.53 | 0.08 | 0.39 | 14.86 | 0.99 |
| TTM-002 | 3.55 | 294.36 | 5.80 | 264.86 | 0.33 | 0.69 | 0.08 | 0.32 | 6.92 | 0.49 |
| AAV9 | 0.01 | 1.00 | 0.02 | 1.00 | 0.48 | 1.00 | 0.23 | 1.00 | 13.79 | 1.00 |

TABLE 53
Quantification of transgene mRNA by RT-qPCR following intravenous administration of AAV
particles comprising a TTM-001 capsid or a TTM-002 capsid normalized to the actual titer
of the viral vector in the dosing solution (mRNA = transgene mRNA fold over housekeeping
gene; rel. to AAV9 = transgene mRNA fold over housekeeping gene relative to AAV9)

| | Tissue | | | | | | | | | |
|---|---|---|---|---|---|---|---|---|---|---|
| | Caudate | | Motor Cortex | | Heart | | Muscle | | Liver | |
| | | rel. to | | rel. to | | rel. to | | rel. to | | rel. to |
| Capsid | mRNA | AAV9 | mRNA | AAV9 | mRNA | AAV9 | mRNA | AAV9 | mRNA | AAV9 |
| TTM-001 | 17.56 | 594.71 | 27.80 | 586.23 | 16.05 | 1.40 | 0.26 | 1.19 | 3.23 | 1.93 |
| TTM-002 | 14.21 | 479.39 | 19.73 | 410.40 | 2.78 | 0.27 | 0.06 | 0.46 | 0.62 | 0.36 |
| AAV9 | 0.03 | 1.00 | 0.05 | 1.00 | 12.67 | 1.00 | 0.15 | 1.00 | 1.85 | 1.00 |

These data in marmosets for TTM-002 were similar to those observed in African green monkeys, further demonstrating cross-species compatibility of the TTM-002 capsid variant. Taken together, these data demonstrate that TTM-001 and TTM-002 are enhanced CNS tropic capsids in marmosets. TTM-002 was also detargeted in the liver, heart, and muscle relative to AAV9 in marmosets, where TTM-001 demonstrated comparable biodistribution and/or transgene expression in the liver, heart, and muscle compared to AAV9. Additionally, the TTM-001 and TTM-002 capsid variants were able to successfully penetrate the blood brain barrier following intravenous injection.
C. Evaluation of(TTM-001, TTM-002, TTM-003, TTM-006, and TTM-007 in Cynomolgus macaques (Macaca fascicularis)

AAV particles were generated with the TTM4002 capsid variant, the TTM401 capsid variant, the TTM-003 capsid variant, the TTM-006 capsid variant, the TTM-027 capsid variant, or the AAV9 capsid control, each of which comprised a self-complementary viral genome encoding a histone H2b protein driven by a ubiquitous CAG promoter. The AAV particles comprising the TTM-002 capsid variant, the TTM-001 capsid variant, the TTM427 capsid variant or the AAV9 capsid control were administered to a first group of male Cynomolgus macaques (Macaca fascicularis: 4-6 kg body weight; over 2 years old) intravenously in a single solution, at a total dose per group of 2e13 VG/kg or a dose per capsid of 4e12 VG/kg. The AAV particles comprising the TTM-003 capsid variant or the TTM-006 capsid variant were administered to a second group of male cynomolgus macaques (Macaca fascicularis; 4-6 kg body weight; over 2 years old) intravenously in a single solution, at a total dose per group of 2e13 VG/kg or a dose per capsid of 4e12 VG/kg. The in-life period was 28 days for both groups, and then various CNS and peripheral tissues were collected for measuring transgene mRNA (expression) by RT-qPCR: protein expression by 1HC/chromogenic staining (e.g., DAB staining for percent of DAB+ cells indicating the percent of cells transduced); percent positive cells (e.g., neurons, motor neurons, and astrocytes) in brain and spinal cord regions by immunofluorescence microscopy; and viral DNA (biodistribution) by ddPCR.

As shown in Table 54, TTM-001, TTM-002, TTM-003, TTM-006, and TTM-027 demonstrated increased CNS transduction and/or biodistribution in several regions of the brain (greater than 30% of cells transduced observed in several regions for multiple capsid variants) and spinal cord of the Cynomolgus macaques after intravenous administration at a relatively low dose of 4e12 vg/kg. More specifically, TTM-003 was capable of transducing up to 40% of cells in the caudate, putamen, and cortex: TTM-027 was capable of transducing up to 30% of cells in the caudate, putamen, and cortex; and both showed improved delivery to the spinal cord relative to AAV9 and TTM4002, at a dose of 4e12 vg/kg.

Cell-typing was also performed in the putamen, substantia nigra, and temporal cortex of the brain to measure the percent of neurons (NeuN+ cells) and astrocytes (Sox9+ cells) that were transduced by the AAV particles comprising the TTM403 and TTM-027 capsid variants or the AAV9 controls (Table 55). TTM-003 was capable of transducing up to 47.8% of neurons and 79.5% of astrocytes in the putamen: 25.3% of neurons and 87.5% of astrocytes in the temporal cortex; and 33.7% of neurons and 18.6% of astrocytes in the substantia nigra (Table 55). TTM-027 was capable of transducing up to 27% of neurons and 41.8% of astrocytes in the putamen; 12.3% of neurons and 51.4% of astrocytes in the temporal cortex; and 21.1% of neurons and 12.2% of astrocytes in the substantia nigra (Table 55). Co-localization of TTM4027 and TTM-003 with motor neurons (ChAT+ cells) was also observed in the spinal cord by immunofluorescence microscopy (Table 55), Across the lumbar, cervical, and thoracic spinal cord. TTM4003 was capable of transducing 78.5% of motor neurons and TTM-027 was capable of transducing 53.5% of motor neurons (Table 55).

In the peripheral tissues, all of the TTM-001, TTM-002, TTM-003, TTM-006, and TTM-007 capsid variants tested exhibited robust liver de-targeting relative to AAV9 (Table 56).

TABLE 54
Quantification of viral genome copies per diploid genome (vg/dg)
(biodistribution) by ddPCR, transgene mRNA by RT-qPCR (mRNA =
transgene mRNA fold over housekeeping gene), and percent
of DAB+ cells in tissues of the CNS of cynomolgus macaques

| | Putamen | | |
|---|---|---|---|
| Capsid | vg/dg | mRNA | % DAB+ Cells |
| AAV9 | 0.1 | 0.03 | 2 |
| TTM-001 | 0.2 | 0.05 | 5 |
| TTM-002 | 0.4 | 0.2 | 13 |
| TTM-027 | 1.2 | 0.83 | 29 |
| TTM-006 | 0.7 | 0.19 | 15 |
| TTM-003 | 1.1 | 1.69 | 39 |
| | Caudate | | |
| Capsid | vg/dg | mRNA | % DAB+ Cells |
| AAV9 | 0.04 | 0.02 | 2 |
| TTM-001 | 0.16 | 0.03 | 5 |
| TTM-002 | 0.42 | 0.15 | 14 |
| TTM-027 | 1.09 | 0.67 | 23 |
| TTM-006 | 0.74 | 0.64 | 27 |
| TTM-003 | 1.09 | 2.99 | 41 |
| | Motor Cortex | | |
| Capsid | vg/dg | mRNA | % DAB+ Cells |
| AAV9 | 0.1 | 0.02 | 1 |
| TTM-001 | 0.3 | 0.02 | 4 |
| TTM-002 | 0.7 | 0.07 | 9 |
| TTM-027 | 1.5 | 1.38 | 22 |
| TTM-006 | 0.8 | 1.36 | 14 |
| TTM-003 | 1.2 | 7.43 | 40 |
| | Cervical (C3) Spinal Cord | | |
| | vg/dg | mRNA | % DAB+ Cells |
| Capsid | (ventral horn) | (ventral horn) | (grey matter) |
| AAV9 | 0.1 | 0.4 | 2 |
| TTM-001 | 0.1 | 0.1 | 2 |
| TTM-002 | 0.3 | 0.5 | 8 |
| TTM-027 | 0.6 | 1.9 | 8 |
| TTM-006 | 0.9 | 3.1 | 15 |
| TTM-003 | 1.0 | 10.7 | 16 |
| | Cervical Dorsal Root Ganglion | | |
| Capsid | vg/dg | mRNA | % DAB+ Cells |
| AAV9 | 0.10 | 3.9 | 1 |
| TTM-001 | 0.04 | 0.3 | 2 |
| TTM-002 | 0.04 | 1.0 | 2 |
| TTM-027 | 0.05 | 1.6 | 1 |
| TTM-006 | 0.18 | 7.4 | 2 |
| TTM-003 | 0.12 | 21.2 | 1 |

TABLE 55
Quantification of neurons (% NeuN positive cells), motor neurons
(% chAT positive cell) and/or astrocytes (% Sox9 cells) transduced
with the TTM-003 and TTM-027 capsid variants in the putamen,
temporal cortex, substantia nigra, and spinal cord

| | Putamen | |
|---|---|---|
| Capsid | % NeuN Positive Cells | % Sox9 Positive Cells |
| AAV9 (control 1) | 0.2 | 2.7 |
| AAV9 (control 2) | 0.5 | 6.5 |
| TTM-003 | 47.8 | 79.5 |
| TTM-027 | 27.0 | 41.8 |
| | Temporal Cortex | |
| Capsid | % NeuN Positive Cells | % Sox9 Positive Cells |
| AAV9 (control 1) | 0.1 | 2.7 |
| AAV9 (control 2) | 0.1 | 4.9 |
| TTM-003 | 25.3 | 87.5 |
| TTM-027 | 12.3 | 51.4 |
| | Substantia Nigra | |
| Capsid | % NeuN Positive Cells | % Sox9 Positive Cells |
| AAV9 (control 1) | 2.7 | 2.8 |
| AAV9 (control 2) | 1.6 | 7.0 |
| TTM-003 | 33.7 | 18.6 |
| TTM-027 | 21.1 | 12.2 |

| | Lumbar, Cervical, and Thoracic Spinal Cord |
|---|---|
| Capsid | % ChAT Positive Cells |
| AAV9 (control 1) | 14.9 |
| AAV9 (control 2) | 23.8 |
| TTM-003 | 78.5 |
| TTM-027 | 53.5 |

TABLE 56
Quantification of viral genome copies per diploid genome (vg/dg)
(biodistribution) by ddPCR, transgene mRNA by RT-qPCR (mRNA =
transgene mRNA fold over housekeeping gene), and percent of DAB+
cells in the peripheral tissues of cynomolgus macaques

| | Liver | | |
|---|---|---|---|
| Capsid | vg/dg | mRNA | % DAB+ Cells |
| AAV9 | 147 | 8.9 | 93 |
| TTM-001 | 15 | 0.2 | 20 |
| TTM-002 | 13 | 0.4 | 32 |
| TTM-027 | 2 | 0.2 | 7 |
| TTM-006 | 9 | 0.4 | 24 |
| TTM-003 | 5 | 0.4 | 24 |
| | Heart | | |
| Capsid | vg/dg | mRNA | % DAB+ Cells |
| AAV9 | 2.2 | 25.8 | 27 |
| TTM-001 | 0.4 | 3.5 | 10 |
| TTM-002 | 1.0 | 8.0 | 20 |
| TTM-027 | 0.6 | 3.4 | 9 |
| TTM-006 | 0.7 | 9.0 | 20 |
| TTM-003 | 0.9 | 23.6 | 16 |

Muscle (Vastus Lateralis)

| Capsid | vg/dg | mRNA |
|---|---|---|
| AAV9 | 0.58 | 1.38 |
| TTM-001 | 0.09 | 0.04 |
| TTM-002 | 0.24 | 0.08 |
| TTM-027 | 0.21 | 0.05 |
| TTM-006 | 0.86 | 9.84 |
| TTM-003 | 1.01 | 10.85 |

Muscle (Gastrocnemius)

| Capsid | vg/dg | mRNA |
|---|---|---|
| AAV9 | 0.56 | 2.13 |
| TTM-001 | 0.08 | 0.05 |
| TTM-002 | 0.22 | 0.13 |
| TTM-027 | 0.18 | 0.05 |
| TTM-006 | 0.51 | 1.04 |
| TTM-003 | 0.58 | 1.57 |

Taken together, these data demonstrate that TTM-001, TTM-002, TTM403, TTM-006, and TTM-027 are enhanced CNS tropic capsids in Cynomolgus macaques that were capable of crossing the blood brain barrier following intravenous injection, even at a low dose of 4e12 vg/kg. TTM-003 and TTM-027 were also capable of transducing both neurons and astrocytes in several brain tissues as well as motor neurons in the spinal cord. All capsids variants also demonstrated robust liver de-targeting relative to AAV9.

D. Individual Evaluation of TTM-003 in Cynomolgus macaques (Macaca fascicularis)

AAV particles were generated with the TM-003 capsid variant which comprised a self-complementary viral genome encoding a histone H2b protein with an HA tag driven by a ubiquitous CAG promoter. The AAV particles comprising the TTM-003 capsid variant were administered to Cynomolgus macaques (Macaca fascicularis) (n=3 male monkeys: 4-12 years of age) intravenously at a dose of 3e13 VG/kg. The in-life period was 28 days and then various CNS and peripheral tissues were collected for measuring transgene mRNA (expression) by RT-ddPCR, viral DNA (biodistribution) by ddPCR, and immunohistochemistry (IHC)/chromogenic quantification of percent positive cells in various tissues (cellular tropism).

As shown in Table 40, substantial and widespread transduction of TTM-003 was observed in the brain and spinal cord in NHPs following intravenous administration of the AAV particles comprising the TTM-003 capsid variant. When administered at a dose of 3e13 VG/kg. TTM-03 was capable of transducing multiple cell types in the brain and spinal cord, including neurons and astrocytes, as quantified in Table 40. Distribution and transgene expression was also measured in the peripheral tissues of liver, heart, and muscle as provided in Table 41.

Taken together, these data demonstrate that TTM-003 is a CNS-tropic capsid in NHPs (Cynomolgus macaques) that can infect both neuronal and non-neuronal cells. Additionally, the TTM-003 capsid variant was able to successfully penetrate the blood brain barrier following intravenous injection.

TABLE 40
Quantification of viral genome copies per diploid genome
(vg/dg) (biodistribution) by ddPCR; transgene mRNA expression
by RT-ddPCR (mRNA = transgene mRNA expression relative
to a housekeeping gene (mTBP)); and the percentage of cells
transduced with the TTM-003 capsid variant measured by co-
localized staining of HA (payload tag) and DAB, NeuN (neurons),
or Sox9 (astrocytes) in the brain and spinal cord of NHPs
(each value represents the average from 3 NHPs)

| | | | % HA and | % HA and | % HA and |
|---|---|---|---|---|---|
| | | | DAB | NeuN | Sox9 |
| | | | Positive | Positive | Positive |
| Tissue | vg/dg | mRNA | Cells | Neurons | Astrocytes |
| Frontal Cortex | 7.5 | 1.4 | 40 | 8.3 | 86.2 |
| Motor Cortex | 6.5 | 7.4 | 60 | 13.7 | 88.4 |
| Temporal Cortex | 4.5 | 0.9 | 47 | 13.4 | 95.1 |
| Entorhinal Cortex | — | — | 34 | 5.9 | 87.8 |
| Hippocampus | 3.3 | 1.5 | 34 | 11.2 | 79.2 |
| Thalamus | 7.6 | 3.1 | 46 | 40.8 | 91.8 |
| Caudate | 7 | 2.5 | 55 | — | — |
| Putamen | 5.3 | 1.8 | 52 | 30.8 | 88 |
| Substantia Nigra | 3.1 | 1.5 | 19 | 13.1 | 45.5 |
| Lateral Geniculate | — | — | 74 | — | — |
| Nucleus (LGN) | | | | | |
| Dentate Nucleus | 2.1 | 1.9 | 38 | — | — |
| Cerebellar Cortex | 1.4 | 2.9 | — | — | — |
| Spinal Cord - | 4.3 | 8.2 | 20 | — | 78.4 |
| Cervical | | | | | |
| Spinal Cord - | — | — | 28 | — | 85.9 |
| Thoracic | | | | | |
| Spinal Cord - | — | — | 24 | — | 83.8 |
| Lumbar | | | | | |
| DRG - Cervical | 2.1 | 5.1 | 62 | — | — |
| DRG - Thoracic | 6.2 | 0.9 | 71 | — | — |
| DRG - Lumbar | 2.6 | 4.8 | 65 | — | — |

TABLE 41
Quantification of viral genome copies per diploid genome (vg/dg)
(biodistribution) by ddPCR; transgene mRNA expression by RT-
ddPCR (mRNA = transgene mRNA relative to a housekeeping
gene (mTBP)); and percentage of cells transduced with the
TTM-003 capsid variant measured by co-localized HA (payload
tag) and DAB staining in the peripheral tissues of NHPs (each
value represents the average of three individual NHPs)

| Tissue | vg/dg | mRNA | % HA and DAB Cells |
|---|---|---|---|
| Liver | 52.2 | 0.4 | 48 |
| Heart | 5.3 | 67 | 40 |
| Vastus Lateralis | 1.4 | 0.7 | 43 |
| Gastrocnemius | 1.6 | 10.6 | 39 |

E. Evaluation of TTM-027 in Cynomolgus macaques (Macaca fascicularis)

AAV particles were generated with the TTM-027 capsid variant or the AAV9 capsid control which comprised a self-complementary viral genome encoding a histone H2b protein driven by a ubiquitous CAG promoter. The AAV particles comprising the TTM-027 capsid variant or the AAV9 capsid control were administered to a group of male Cynomolgus macaques (Macaca fascicularis; 8-9 kg body weight; 4-10 years old; n=3) intravenously in a single solution, at a total dose per group of 2e 13 VG/kg or a dose per capsid of 4e 12 VG/kg. The in-life period was 28 days, and then various CNS and peripheral tissues were collected for measuring transgene mRNA (expression) by RT-ddPCR; protein expression by IHC/chromogenic staining and quantification of percent positive cells, and viral DNA (biodistribution) by ddPCR.

As shown in Table 42, TIM-027 demonstrated increased CNS transduction in several brain regions and the spinal cord of the Cynomolgus macaques after intravenous administration at a relatively low dose of 4c 12 vg/kg.

TABLE 42
Quantification of percent DAB+ cells; viral genome copies
per diploid genome (vg/dg) (biodistribution) by ddPCR;
and transgene mRNA expression by RT-ddPCR (mRNA =
transgene mRNA expression relative to a housekeeping gene
(mTBP)) in tissues of the CNS of cynomolgus macaques

| | % DAB Positive Cells | | |
|---|---|---|---|
| | | | % DAB positive cells |
| Tissue | AAV9 | TTM-027 | relative to AAV9 |
| Caudate | 2 | 46 | 23 |
| Putamen | 2 | 45 | 22.5 |
| Thalamus | 2 | 36 | 18 |
| Substantia Nigra | 2 | 15 | 7.5 |
| Hippocampus | 1 | 32 | 32 |
| Entorhinal Cortex | 2 | 30 | 15 |
| Temporal Cortex | 1 | 41 | 41 |
| Primary Motor Cortex | 4 | 47 | 11.75 |
| Dentate Nucleus | 8 | 37 | 4.63 |
| Lateral Geniculate | 8 | 55 | 6.88 |
| Nucleus (LGN) | | | |
| Cervical Spinal Cord | 10 | 22 | 2.2 |
| Thoracic Spinal Cord | 14 | 20 | 1.43 |
| Lumbar Spinal Cord | 13 | 21 | 1.62 |
| Cervical DRG | 57 | 59 | 1.04 |
| Thoracic DRG | 50 | 49 | 0.98 |
| Lumbar DRG | 51 | 58 | 1.14 |
| | vg/dg | | |
| Tissue | AAV9 | TTM-027 | vg/dg relative to AAV9 |
| Caudate | 0.1 | 2.9 | 29.00 |
| Putamen | 0.1 | 2.3 | 23.00 |
| Primary Motor Cortex | 0.2 | 2.4 | 12.00 |
| Cervical Spinal Cord | 0.4 | 3.0 | 7.50 |
| Cervical DRG | 1.4 | 0.1 | 0.07 |
| | mRNA | | |
| Tissue | AAV9 | TTM-027 | mRNA relative to AAV9 |
| Caudate | 0.046 | 2.5 | 54.35 |
| Putamen | 0.1 | 1.0 | 10.00 |
| Cervical DRG | 3.5 | 0.3 | 0.09 |

The percentage of DAB positive cells, biodistribution, and mRNA expression were also quantified in the peripheral tissues of the liver, heart, and muscle, which is provided in Table 43. The TTM-027 capsid variant exhibited robust liver de-targeting relative to AAV9 (Table 43).

TABLE 43
Quantification of percent DAB+ cells; viral genome copies
per diploid genome (vg/dg) (biodistribution) by ddPCR;
and transgene mRNA expression by RT-ddPCR (mRNA =
transgene mRNA expression relative to a housekeeping gene
(mTBP)) in the peripheral tissues of cynomolgus macaques

| | % DAB Positive Cells | | |
|---|---|---|---|
| | | | % DAB positive cells |
| Tissue | AAV9 | TTM-027 | relative to AAV9 |
| Liver | 65 | 11 | 0.17 |
| Heart | 19 | 15 | 0.79 |
| Gastrocnemius Muscle | 0 | 16 | 1.78 |
| Vastus Lateralis | 21 | 13 | 0.62 |
| | vg/dg | | |
| Tissue | AAV9 | TTM-027 | vg/dg relative to AAV9 |
| Liver | 113.9 | 1.0 | 0.01 |
| Heart | 2.2 | 0.1 | 0.05 |
| Gastrocnemius Muscle | 1.07 | 0.04 | 0.04 |
| Vastus Lateralis | 1.7 | 0.1 | 0.06 |
| | mRNA | | |
| Tissue | AAV9 | TTM-027 | mRNA relative to AAV9 |
| Liver | 19.6 | 2.1 | 0.11 |
| Heart | 33.0 | 1.1 | 0.03 |
| Gastrocnemius Muscle | 6.29 | 0.51 | 0.08 |
| Vastus Lateralis | 0.51 | 0.02 | 0.04 |

Taken together, these data demonstrate that TTM-027 is an enhanced CNS tropic capsid in Cynomolgus macaques that was capable of crossing the blood brain barrier following intravenous injection, even at a low dose of 4e12 vg/kg, which is consistent with what was observed in Example 8C above.

F. Individual Evaluation of 7TM-027 in Cynomolgus macaques (Macaca fascicularis)

AAV particles were generated with the TTM-027 capsid variant which each comprised a self-complementary viral genome encoding a histone H2b protein with an HA tag driven by a ubiquitous CAG promoter. The AAV particles comprising the TM-027 capsid variant were administered to Cynomolgus macaques (Macaca fascicularis) (n=3 male monkeys: 7.4-11 years of age) intravenously at a dose of 3e13 VG/kg. The in-life period was 28 days and then various CNS and peripheral tissues were collected for measuring transgene mRNA (expression) by RT-qPCR, viral DNA (biodistribution) by ddPCR, and immunohistochemistry (IHC)/chromogenic and immunofluorescent quantification of percent positive cells in various tissues (cellular tropism).

As shown in Table 44, substantial and widespread transduction of TTM-027 was observed in the brain and spinal cord of NHPs following intravenous administration of the AAV particles comprising the TTM-027 capsid variant. More specifically, TTM-027 demonstrated superior viral genome biodistribution in a variety of CNS tissues and regions, broader expression in the CNS as shown by both transgene mRNA expression and IHC (Table 44 and Table 45), as well as a highly neurotropic and astrocytic tropism in the brain and the spinal cord (Table 45 and Table 60). TTM-027 when administered intravenously at a dose of 3e13 vg/kg was capable of transducing up to 21-65% of neurons (HA and NeuN positive cells) and 87-97% of astrocytes (HA and Sox9 positive cells) in multiple brain regions; up to 96% of Purkinje Neurons in the cerebellum; up to 84-94% of motor neurons (HA and ChAT positive cells) in the spinal cord; and up to 93-97% of astrocytes (HA and Sox9 positive cells) in the spinal cord (Table 45). TTM-027 was also able to transduce 97.9% of the dopaminergic neurons in the substantia nigra, as indicated by cells that were positive for both tyrosine hydroxylase (TH) and HA (payload tag). The TTM-027 capsid variant was well tolerated in the NHPs.

TABLE 44
Quantification of viral genome copies per diploid genome (vg/dg)
(biodistribution) by ddPCR; transgene mRNA expression by RT-
qPCR (mRNA = transgene mRNA expression relative to a housekeeping
gene (mTBP)); and the percentage of cells transduced with
the TTM-027 capsid variant measured by co-localized staining
of HA (payload tag) and DAB in the brain and spinal cord of
NHPs (each value represents the average from 3 NHPs; for regions
of the DRG, sensory neuron data are shown)

| | | | % HA and DAB |
|---|---|---|---|
| Tissue | vg/dg | mRNA | Positive Cells |
| Frontal Cortex | 6.1 | 6.1 | 53.8 |
| Motor Cortex | 7.3 | 3.1 | 68.1 |
| Temporal Cortex | 1.7 | 3.8 | 51.8 |
| Hippocampus | 5.4 | 1.8 | 34.7 |
| Thalamus | 8.7 | 5.1 | 50.3 |
| Caudate | 7.6 | 5.7 | 57.5 |
| Putamen | 6.0 | 1.8 | 59.1 |
| Substantia Nigra | 2.6 | 5.4 | 37.9 |
| Dentate Nucleus | 2.7 | 17.7 | 55.2 |
| Cervical Spinal Cord | 5.3 | 5.9 | 25.0 |
| Lumbar Spinal Cord | 4.4 | 11.4 | 21.3 |
| Cervical DRG | 1.8 | 4.1 | 45.5 |
| Thoracic DRG | 6.9 | 3.1 | 41.0 |
| Lumbar DRG | 2.5 | 3.7 | 41.6 |

| Tissue | % DAB Positive Cells |
|---|---|
| Entorhinal Cortex | 41.0 |
| Lateral geniculate nucleus (LGN) | 74.5 |
| Thoracic Spinal Cord | 23.6 |
| Purkinje Neurons | 95.7 |
| Pontine Nuclei | 52.7 |
| Inferior Olivary Nuclei | 42.6 |

TABLE 45
Quantification of the percentage of cells transduced with the TTM-
027 capsid variant measured by co-localized staining of HA (payload
tag) and either NeuN (neurons) or Sox9 (astrocytes) in the brain
of NHPs (each value represents the average from 3 NHPs)

| | % HA and NeuN Positive | % HA and Sox9 Positive |
|---|---|---|
| Tissue | Neurons | Astrocytes |
| Caudate | 58 | 99 |
| Entorhinal Cortex | 25 | 97 |
| Frontal Cortex | 42 | 99 |
| Hippocampus | 21 | 87 |
| Motor Cortex | 43 | 98 |
| Putamen | 58 | 95 |
| Substantia Nigra | 51 | 89 |
| Temporal Cortex | 31 | 97 |
| Thalamus | 66 | 97 |

TABLE 60
Quantification of the percentage of cells transduced with
the TTM-027 capsid variant measured by co-localized staining
of HA (payload tag) and either ChAT (motor neurons) or Sox9
(astrocytes) in the gray matter of the spinal cord of NHPs
(each value represents the average from 3 NHPs)

| | % HA and ChAT | % HA and Sox9 |
|---|---|---|
| | Positive | Positive |
| Tissue | Neurons | Astrocytes |
| Cervical Spinal Cord (C2) | 84 | 96 |
| Thoracic Spinal Cord (T8) | 86 | 97 |
| Lumbar Spinal Cord (L2) | 94 | 92 |

The biodistribution and mRNA expression following intravenous administration of AAV particle comprising the TTM-027 capsid variant at a dose of 3e13 vg/kg was also measured in the peripheral tissues of the liver, heart, and muscle (vastus lateralis and gastrocnemius) as provided in Table 61, TTM-027 showed very low biodistribution and mRNA expression in the liver in NHPs (Table 61), and demonstrated substantially reduced liver tropism.

TABLE 61
Quantification of viral genome copies per diploid genome (vg/dg)
(biodistribution) by ddPCR, and transgene mRNA expression relative
to a housekeeping gene (mTBP) by RT-ddPCR in the peripheral tissues
of NHPs (each value represents the average from 3 NHPs)

| | | Transgene mRNA expression | |
|---|---|---|---|
| | | relative to a | % DAB |
| Tissue | vg/dg | housekeeping gene (mTBP) | Positive Cells |
| Liver | 13.9 | 0.5 | 56.1 |
| Heart | 2.7 | 8.0 | 38.2 |
| Vastus Lateralis | 1.5 | 2.2 | 18.3 |
| Gastrocnemius | 2.2 | 9.6 | 38.9 |

Taken together, the individual characterization of the TTM-027 capsid variant further demonstrates and confirms that the TTM-027 is an enhanced CNS tropic capsid in cynomolgus macaques, capable of crossing the blood brain barrier following intravenous injection, consistent with what was observed in Examples 8C and 8E above.

### Example 9. Dose Response Evaluation of the TTM-002 and TTM-027 AAV Capsid Variants

This Example investigates transduction of the TTM-002 (SEQ ID NO: 982 (amino acid) and SEQ ID NO: 984 (DNA), comprising SEQ ID NO: 2) and TTM-027 (SEQ ID NO: 4 (amino acid) and SEQ ID NO: 5 (DNA), comprising SEQ ID NO: 3272) capsid variants following intravenous administration at increasing doses in mice.

AAV particles were generated with the TTM-002 capsid variant or the TTM-027 capsid variant which comprised a single stranded viral genome encoding a histone protein with an HA tag (H3F3-HA) and a woodchuck hepatitis virus posttranscriptional regulatory element (WPRE) driven by a ubiquitous CBA promoter. The AAV particles comprising the TTM-02 capsid variant or the TTM-027 AAV capsid variant were administered to mice (n=3 mice per dosing group; Balb/c; 6-8 weeks of age) via tail vein injection at increasing doses of 1e12 vg/kg, 3.2e12 vg/kg, 1e13 vg/kg, 3.2e13 vg/kg, or 1e14 vg/kg. The dose of 3.2e12 vg/kg was approximately equivalent to the dose used per capsid in the Cynomolgus macaques (Macaca fascicularis) in Example 8C above. The in-life period was 28 days and CNS tissues were collected for measuring transgene mRNA (expression) by qPCR and the percent of HA positive cells in the brain.

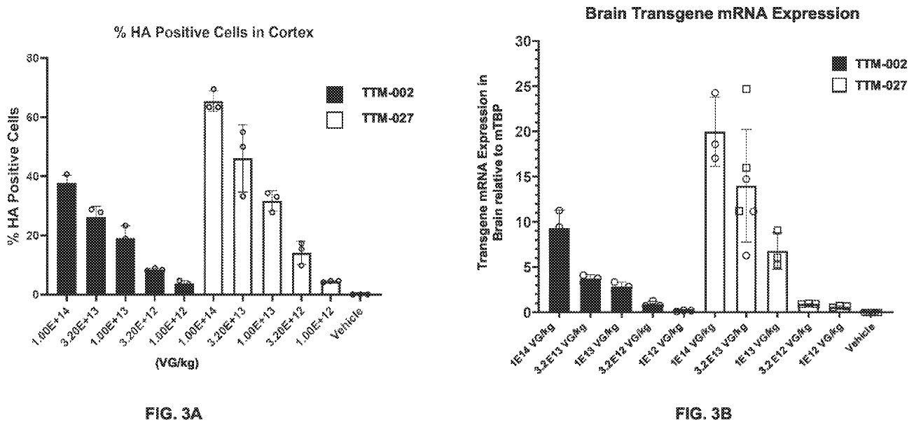

As shown in Table 57 and FIG. 3A, a dose dependent increase in transduction of both the TTM-002 and TTM-027 capsid variants was observed in the mouse cortex following intravenous administration of the AAV particles at the increasing doses. The percent of cells transduced in the mouse cortex with TTM-027 was higher at all doses compared to TTM-02. At the highest dose tested, 1e14 vg/kg. TTM-027 transduced 65% of cells in the cortex, whereas TM-402 transduced 38% of cells, with TTM-002 demonstrating an even distribution between neurons and astrocytes, identified by NeuN and Sox9 markers, respectively (Table 58). It was also observed that an increase in dose from 3.2e12 to 3.2e13 vg/kg resulted in a greater than 3 fold increase in percent positive cells. Consistent with the percentage of TTM-002 or TTM-027 positive cells, a dose dependent increase in transgene mRNA expression was also observed in the mouse brain following intravenous administration of the TTM-002 and TTM-027 capsid variants at the increasing doses (FIG. 3B and Table 57).

Measurements in Table 57 are by co-localization of chromogenic HA staining and hematoxylin, as well as quantification by qPCR of transgene mRNA expression relative to a housekeeping gene (nTBP) in the mouse brain, following intravenous administration (each value is the average of three individual measurements within each cortex taken from three mice). Measurements in Table 58 are in the mouse cortex by fluorescence microscopy and co-staining with HA and cell-type specific markets (each value is the average of three individual measurements within each cortex taken from three mice).

Taken together, these data demonstrate a dose-dependent response across a 2-log dose range in mouse (0el2 to 1e14 vg/kg), without reaching saturation at the maximal dose (Table 57 and Table 58).

TABLE 57
Quantification of cells positive for the TTM-002 or TTM-
027 capsid variant per mm² (HA positive cells/mm²) or percentage
of cells transduced with the TTM-002 or TTM-027 capsid
variants (% HA positive cells) in the mouse cortex

| | | | | Transgene mRNA |
|---|---|---|---|---|
| | | HA | % HA | expression relative |
| Capsid | Dosage | Positive | Positive | to housekeeping |
| Variant | (vg/kg) | Cells/mm² | Cells | gene |
| TTM-002 | 1e14 | 679.147 | 37.668 | 9.3349 |
| | 3.2e13 | 414.043 | 26.279 | 3.7168 |
| | 1e13 | 277.497 | 19.078 | 2.8445 |
| | 3.2e12 | 151.841 | 8.450 | 0.9074 |
| | 1e12 | 61.018 | 3.616 | 0.1681 |
| Vehicle | 0 | 0.185 | 0.011 | 0.0003 |
| TTM-027 | 1e14 | 1019.777 | 65.451 | 19.9655 |
| | 3.2e13 | 835.525 | 46.076 | 14.0024 |
| | 1e13 | 585.873 | 31.717 | 6.7856 |
| | 3.2e12 | 301.636 | 14.169 | 0.9269 |
| | 1e12 | 78.350 | 4.427 | 0.6379 |

TABLE 58
Quantification of total cells (HA positive cells/mm²), neurons (NeuN positive cells that are
also HA positive/mm²), and astrocytes (Sox9 positive cells that are also HA positive/mm²) positive
for the TTM-002 capsid variant per mm² or percentage of total cells (% HA positive cells),
neurons (% NeuN positive cells that are also HA positive), and astrocytes (% Sox9 positive
cells that are also HA positive) transduced with the TTM-002 capsid variant

| | | | | NeuN | % NeuN | Sox9 | % Sox9 |
|---|---|---|---|---|---|---|---|
| | | | | Positive | Positive | Positive | Positive |
| | | HA | % HA | Cells that | Cells that | Cells that | Cells that |
| Capsid | Dosage | Positive | Positive | are also HA | are also | are also HA | are also |
| Variant | (vg/kg) | Cells/mm² | Cells | positive/mm² | HA positive | positive/mm² | HA positive |
| TTM-002 | 1e14 | 495.9 | 28.6 | 154.7 | 13.5 | 26.2 | 12.8 |
| | 3.2e13 | 344.0 | 18.8 | 126.8 | 9.2 | 19.8 | 14.6 |
| | 1e13 | 270.7 | 15.6 | 73.2 | 5.5 | 12.0 | 8.0 |
| | 3.2e12 | 176.9 | 8.2 | 59.2 | 3.6 | 8.5 | 4.4 |
| | 1e12 | 57.9 | 3.5 | 7.3 | 0.6 | 4.4 | 2.3 |
| Vehicle | 0 | 0 | 0 | 0 | 0 | 0 | 0 |

Example 10: Individual Characterization of TTM-002 and TTM-001 Capsid Variants in Mice

The goal of these experiments was to determine the transduction level, tropism, ability to cross the blood brain barrier, and overall spatial distribution in the brain, heart, and liver of the TTM-001 and TTM-002 capsids and variants thereof relative to AAV9 following intravenous injection in mice. TTM-001 (SEQ ID NO: 981), TTM-002 (SEQ ID NO: 982), and variants of the TTM-002 and TTM-001 capsids comprising local modifications in loop TV were investigated including TTM-003 (SEQ ID NO: 36), TTM-006 (SEQ ID NO: 39), TTM-018 (SEQ ID NO: 51), and TTM-019 (SEQ ID NO: 52). The amino acid sequences for these capsid variants are provided, e.g., in Table 4.

AAV particles were generated with each of these capsid variants encapsulating a fluorescent reporter construct, ZsGreen-HA, driven by a CAG promoter. Each capsid variant and AAV9 control were tested by intravenously administering by tail vein injection, the AAV particle formulation at 1e13 VG/kg to three BALB/c mice. The in-life period was 28 days and then various CNS and peripheral tissues were collected for measuring transgene mRNA expression.

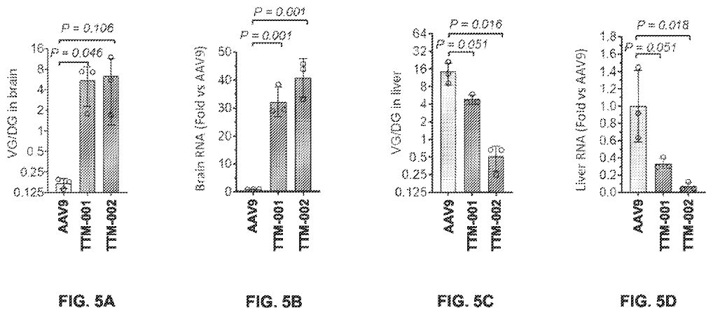

The brains isolated from mice injected with the AAV particles encapsulated in the TTM-002 capsid, the TTM-00 I capsid, or variants of the TTM-00 or TTM-002 capsid comprising local modifications were assayed to calculate ZsGreen expression and/or transgene DNA. Data were provided as fold over the AAV9 control (Table 59), All of the variants of the TTM-001 and TTM-002 capsids as well as TTM-001 and TTM-002 demonstrated increased CNS tropism and expression in the brain relative to AAV9 (Table 59; FIGS. 5A-5B), More specifically, TTM-001 and TTM-002 showed a broad distribution throughout the entire brain and spinal cord, outperforming the AAV9 control by approximately 30- and 40-fold, respectively, in terms of viral DNA biodistribution and transgene RNA expression (FIGS. 5A-5B. Table 59). The variants of the TTM-001 and TTM-002 capsids as well as TTM-001 and TTM-002 also demonstrated reduced mRNA and DNA expression in the liver by qPCR relative to the AAV9 control, with TTM-002 showing 14-fold lower gene expression than the AAV9 control (Table 59; FIGS. 5C-5D), Similar results were observed by immunohistochemistry (IHC) staining of the brain (including the cortex, thalamus, and cerebellum), spinal cord (grey matter), and liver for transduction by AAV particles comprising the AAV capsid variants investigated. Transduction of the heart, skeletal muscle, and kidney did not show major differences between AAV9 and TTM-002.

TABLE 59
ZsGreen Expression and Transgene DNA and/or
RNA expression for variants of the TTM-001
and TTM-002 capsids relative to AAV9 in mice

| | Brain | | Liver | |
|---|---|---|---|---|
| | | Transgene | Transgene | Transgene |
| | ZsGreen Expression | DNA fold | RNA fold | DNA fold |
| | fold change | change | change | change |
| Capsid | relative | relative | relative | relative |
| Variant | to AAV9 | to AAV9 | to AAV9 | to AAV9 |
| AAV9 | 1.0 | 1.0 | 1.000 | 1.000 |
| TTM-001 | 32.3 | 31.9 | 0.337 | 0.348 |
| TTM-002 | 40.9 | 37.0 | 0.072 | 0.034 |
| TTM-006 | 16.1 | 36.9 | 0.036 | 0.013 |
| TTM-018 | 22.8 | 27.0 | 0.231 | 0.223 |
| TTM-019 | 14.6 | 14.4 | 0.117 | 0.112 |
| TTM-003 | 17.3 | 14.6 | 0.015 | 0.026 |

### Example 11: In Vivo Evaluation of TTM-002 AAV Capsid Variant Comprising an HA-Tagged Nucleotide Sequence Encoding GBA in Cynomolgus Monkeys

This Example investigates the distribution and efficacy of an AAV comprising a TTM-002 AAV capsid variant (SEQ ID NO: 982) and a codon-optimized nucleotide sequence (SEQ ID NO: 1773) encoding a GBA and an HA-tag (TTM-002.GBA_VG17-HA), Mauritius male cynomolgus monkeys (Macaca fascicularis; 3-6 years of age; 3-8 kg in weight; n=3 per group) were administered TTM-02.GBA_VG17-HA at 3e12 vg/kg or 1e13 vg/kg by intravenous administration. Monkeys were also administered a vehicle control (modified PBS) or 1el3 vg/kg of an AAV9 capsid comprising the same payload construct was used as control (AAV9.GBA_VG17-HA) for comparison. Biodistribution of GBA 1-HA protein was assessed following a 28-day treatment period. Primary readouts included biochemical analysis of viral genomes and mRNA, immunohistochemical analysis of HA levels, and histopathology readouts in the brain, spinal cord, liver, kidney, and heart tissues. Secondary readouts included biochemical analysis of GCase activity, LC/MS-MS analysis of GBA1 levels, and cage-side observations including weekly body weight.

Histopathology analysis revealed no signs of toxicity in the kidneys, hearts, brains, and spinal cords of the treated monkeys at both AAV dosages.

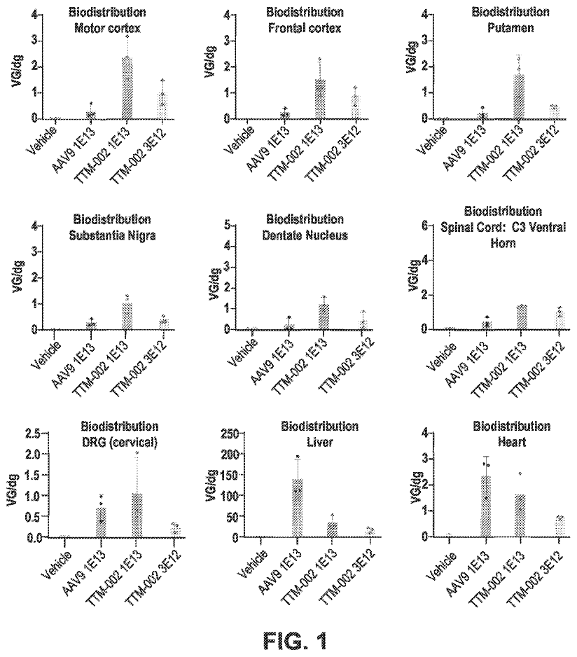

TTM-002.GBA_VG17-HA led to increased viral genome biodistribution in all CNS tissues (FIG. 1; Table 46A-H) and reduced liver transduction as compared to AAV9.GBA_VG17-HA.

TABLE 46A
Viral genome biodistribution in the putamen

| | Animal | Group | Fold over |
|---|---|---|---|
| Treatment | Average | Average | AAV9 |
| Vehicle | 0.00 | 0.00 | |
| | 0.01 | | |
| | 0.00 | | |
| AAV9.GBA_VG17-HA | 0.17 | 0.23 | 1.00 |
| 1e13 vg/kg | 0.44 | | |
| | 0.07 | | |
| TTM-002.GBA_VG17-HA | 0.83 | 1.68 | 7.47 |
| high dose 1e13 vg/kg | 1.92 | | |
| | 2.30 | | |
| TTM-002.GBA_VG17-HA | 0.39 | 0.46 | |
| low dose 3e12 vg/kg | 0.48 | | |
| | 0.51 | | |

TABLE 46B
Viral genome biodistribution in the motor cortex

| | Animal | Group | Fold over |
|---|---|---|---|
| Treatment | Average | Average | AAV9 |
| Vehicle | 0.01 | 0.01 | |
| | 0.00 | | |
| | 0.00 | | |
| AAV9.GBA_VG17-HA | 0.14 | 0.31 | 1.00 |
| 1e13 vg/kg | 0.60 | | |
| | 0.19 | | |
| TTM-002.GBA_VG17-HA | 3.19 | 2.36 | 7.65 |
| high dose 1e13 vg/kg | 1.53 | | |
| | 2.37 | | |
| TTM-002.GBA_VG17-HA | 0.57 | 1.00 | |
| low dose 3e12 vg/kg | 0.94 | | |
| | 1.50 | | |

TABLE 46C
Viral genome biodistribution in the frontal cortex

| | Animal | Group | Fold over |
|---|---|---|---|
| Treatment | Average | Average | AAV9 |
| Vehicle | 0.00 | 0.00 | |
| | 0.00 | | |
| | 0.00 | | |
| AAV9.GBA_VG17-HA | 0.14 | 0.25 | 1.00 |
| 1e13 vg/kg | 0.42 | | |
| | 0.18 | | |
| TTM-002.GBA_VG17-HA | 1.14 | 1.54 | 6.27 |
| high dose 1e13 vg/kg | 1.19 | | |
| | 2.30 | | |
| TTM-002.GBA_VG17-HA | 0.51 | 0.86 | |
| low dose 3e12 vg/kg | 0.87 | | |
| | 1.22 | | |

TABLE 46D
Viral genome biodistribution in the substantia nigra

| | Animal | Group | Fold over |
|---|---|---|---|
| Treatment | Average | Average | AAV9 |
| Vehicle | 0.00 | 0.00 | |
| | 0.00 | | |
| | 0.00 | | |
| AAV9.GBA_VG17-HA | 0.22 | 0.28 | 1.00 |
| 1e13 vg/kg | 0.43 | | |
| | 0.17 | | |
| TTM-002.GBA_VG17-HA | 1.20 | 1.07 | 3.88 |
| high dose 1e13 vg/kg | 0.67 | | |
| | 1.34 | | |
| TTM-002.GBA_VG17-HA | 0.37 | 0.43 | |
| low dose 3e12 vg/kg | 0.34 | | |
| | 0.57 | | |

TABLE 46E
Viral genome biodistribution in the C3 Ventral Horn

| | Animal | Group | Fold over |
|---|---|---|---|
| Treatment | Average | Average | AAV9 |
| Vehicle | 0.04 | 0.03 | |
| | 0.02 | | |
| | 0.03 | | |
| AAV9.GBA_VG17-HA | 0.37 | 0.45 | 1.00 |
| 1e13 vg/kg | 0.74 | | |
| | 0.24 | | |
| TTM-002.GBA_VG17-HA | 1.37 | 1.33 | 2.98 |
| high dose 1e13 vg/kg | 1.26 | | |
| | 1.37 | | |
| TTM-002.GBA_VG17-HA | 0.77 | 1.03 | |
| low dose 3e12 vg/kg | 1.27 | | |
| | 1.05 | | |

TABLE 46F
Viral genome biodistribution in the DRG

| | Animal | Group | Fold over |
|---|---|---|---|
| Treatment | Average | Average | AAV9 |
| Vehicle | 0.01 | 0.00 | |
| | 0.00 | | |
| | 0.00 | | |
| AAV9.GBA_VG17-HA | 0.81 | 0.72 | 1.00 |
| 1e13 vg/kg | 0.98 | | |
| | 0.38 | | |
| TTM-002.GBA_VG17-HA | 2.04 | 0.55 | 0.76 |
| high dose 1e13 vg/kg | 0.65 | | |
| | 0.48 | | |
| TTM-002.GBA_VG17-HA | 0.28 | 0.23 | |
| low dose 3e12 vg/kg | 0.11 | | |
| | 0.32 | | |

TABLE 46G
Viral genome biodistribution in the liver

| | Animal | Group | Fold over |
|---|---|---|---|
| Treatment | Average | Average | AAV9 |
| Vehicle | 0.00 | 0.00 | |
| | 0.00 | | |
| | 0.01 | | |
| AAV9.GBA_VG17-HA | 195.49 | 140.22 | 1.00 |
| 1e13 vg/kg | 111.71 | | |
| | 113.48 | | |
| TTM-002.GBA_VG17-HA | 54.48 | 36.68 | 0.26 |
| high dose 1e13 vg/kg | 28.26 | | |
| | 27.31 | | |
| TTM-002.GBA_VG17-HA | 21.65 | 16.12 | |
| low dose 3e12 vg/kg | 9.07 | | |
| | 17.66 | | |

TABLE 46H
Viral genome biodistribution in the heart

| | Animal | Group | Fold over |
|---|---|---|---|
| Treatment | Ave | Ave | AAV9 |
| Vehicle | 0.00 | 0.04 | |
| | 0.00 | | |
| | 0.12 | | |
| AAV9.GBA_VG17-HA | 2.78 | 2.38 | 1.00 |
| 1e13 vg/kg | 2.84 | | |
| | 1.51 | | |
| TTM-002.GBA_VG17-HA | 2.45 | 1.66 | 0.70 |
| high dose 1e13 vg/kg | 1.46 | | |
| | 1.08 | | |
| TTM-002.GBA_VG17-HA | 0.65 | 0.73 | |
| low dose 3e12 vg/kg | 0.77 | | |
| | 0.78 | | |

TTM e02.GBA_VG17-HA led to increased humanGBA1 mRNA expression over the expression of the TBP housekeeping gene into the dentate nucleus, putamen substantia nig 9 motor and frontal cortex, and spinal cord (Tables 47A-E). Compared to animals treated with AAV9.GBA_VG17-HA, comparable levels of human GBA 1 mRNA expression were observed in the DRG, liver, or heart of animals treated with TTM-002.GBA_VG 17-HA (Tables 47F-H).

TABLE 47A
GBA1 mRNA expression relative to TBP in the frontal cortex

| | | Group Ave | |
|---|---|---|---|
| | FC | FC Glox4 | Fold over |
| Treatment | animal | rel TBP | AAV9 |
| Vehicle | 0.00 | 0.00 | |
| | 0.00 | | |
| | 0.00 | | |
| AAV9.GBA_VG17-HA | 0.05 | 0.04 | |
| 1e13 vg/kg | 0.06 | | |
| | 0.01 | | |
| TTM-002.GBA_VG17-HA | 0.34 | 0.65 | 16.65 |
| high dose 1e13 vg/kg | 0.91 | | |
| | 0.68 | | |
| TTM-002.GBA_VG17-HA | 0.05 | 0.38 | 9.71 |
| low dose 3e12 vg/kg | 0.52 | | |
| | 0.55 | | |

TABLE 47B
GBA1 mRNA expression relative to TBP in the motor cortex

| | | Group Ave | |
|---|---|---|---|
| | FC | FC Glox4 | Fold over |
| Treatment | animal | rel TBP | AAV9 |
| Vehicle | 0.00 | 0.02 | |
| | 0.00 | | |
| | 0.07 | | |
| AAV9.GBA_VG17-HA | 0.02 | 0.03 | |
| 1e13 vg/kg | 0.05 | | |
| | 0.01 | | |
| TTM-002.GBA_VG17-HA | 0.28 | 0.43 | 16.60 |
| high dose 1e13 vg/kg | 0.31 | | |
| | 0.70 | | |
| TTM-002.GBA_VG17-HA | 0.08 | 0.59 | 22.95 |
| low dose 3e12 vg/kg | 0.70 | | |
| | 1.00 | | |

TABLE 47C
GBA1 mRNA expression relative to TBP in the putamen

| | | Group Ave | |
|---|---|---|---|
| | FC | FC Glox4 | Fold over |
| Treatment | animal | rel TBP | AAV9 |
| Vehicle | BLQ | BLQ | |
| | BLQ | | |
| | BLQ | | |
| AAV9.GBA_VG17-HA | 0.04 | 0.05 | |
| 1e13 vg/kg | 0.11 | | |
| | 0.01 | | |
| TTM-002.GBA_VG17-HA | 0.26 | 1.26 | 23.51 |
| high dose 1e13 vg/kg | 2.12 | | |
| | 1.41 | | |
| TTM-002.GBA_VG17-HA | 0.07 | 0.16 | 3.02 |
| low dose 3e12 vg/kg | | | |

TABLE 47D
GBA1 mRNA expression relative to TBP in the substantia nigra

| | | | Fold |
|---|---|---|---|
| | FC | Group Ave FC | over |
| Treatment | animal | Glox4 rel TBP | AAV9 |
| Vehicle | BLQ | BLQ | |
| | BLQ | | |
| | BLQ | | |
| AAV9.GBA_VG17-HA | 0.08 | 0.14 | |
| 1e13 vg/kg | 0.32 | | |
| | 0.03 | | |
| TTM-002.GBA_VG17-HA | 0.42 | 0.84 | 5.85 |
| high dose 1e13 vg/kg | 0.39 | | |
| | 1.72 | | |
| TTM-002.GBA_VG17-HA | 0.14 | 0.25 | 1.77 |
| low dose 3e12 vg/kg | | | |

TABLE 47E
GBA1 mRNA expression relative to TBP in the spinal cord

| | | | Fold |
|---|---|---|---|
| | FC | Group Ave FC | over |
| Treatment | animal | Glox4 rel TBP | AAV9 |
| Vehicle | 0.00 | 0.00 | |
| | 0.00 | | |
| | 0.00 | | |
| AAV9.GBA_VG17-HA | 0.17 | 1.40 | |
| 1e13 vg/kg | 3.74 | | |
| | 0.29 | | |
| TTM-002.GBA_VG17-HA | 1.22 | 2.86 | 2.04 |
| high dose 1e13 vg/kg | 3.85 | | |
| | 3.50 | | |
| TTM-002.GBA_VG17-HA | 2.03 | 3.03 | 2.17 |
| low dose 3e12 vg/kg | | | |

TABLE 47F
GBA1 mRNA expression relative to TBP in the DRG

| | | | Fold |
|---|---|---|---|
| | FC | Group Ave FC | over |
| Treatment | animal | Glox4 rel TBP | AAV9 |
| Vehicle | 0.00 | 0.00 | |
| | 0.00 | | |
| | 0.00 | | |
| AAV9.GBA_VG17-HA | 53.23 | 26.01 | |
| 1e13 vg/kg | 13.21 | | |
| | 11.60 | | |
| TTM-002.GBA_VG17-HA | 22.68 | 22.89 | 0.88 |
| high dose 1e13 vg/kg | 29.26 | | |
| | 16.75 | | |
| TTM-002.GBA_VG17-HA | 6.00 | 7.58 | 0.29 |
| low dose 3e12 vg/kg | | | |

TABLE 47G
GBA1 mRNA expression relative to TBP in the heart

| | | | Fold |
|---|---|---|---|
| | FC | Group Ave FC | over |
| Treatment | animal | Glox4 rel TBP | AAV9 |
| Vehicle | 0.00 | 0.04 | |
| | 0.00 | | |
| | 0.12 | | |
| AAV9.GBA_VG17-HA | 64.58 | 48.99 | |
| 1e13 vg/kg | 88.47 | | |
| | 42.77 | | |
| TTM-002.GBA_VG17-HA | 38.29 | 37.93 | 0.77 |
| high dose 1e13 vg/kg | 61.89 | | |
| | 13.62 | | |
| TTM-002.GBA_VG17-HA | 11.47 | 8.16 | 0.17 |
| low dose 3e12 vg/kg | | | |

TABLE 47H
GBA1 mRNA expression relative to TBP in the liver

| | | | Fold |
|---|---|---|---|
| | FC | Group Ave FC | over |
| Treatment | animal | Glox4 rel TBP | AAV9 |
| Vehicle | 0.00 | 0.00 | |
| | 0.00 | | |
| | 0.00 | | |
| AAV9.GBA_VG17-HA | 6.71 | 5.39 | |
| 1e13 vg/kg | 2.26 | | |
| | 7.20 | | |
| TTM-002.GBA_VG17-HA | 2.88 | 2.37 | 0.44 |
| high dose 1e13 vg/kg | 1.64 | | |
| | 2.57 | | |
| TTM-002.GBA_VG17-HA | 6.60 | 3.28 | 0.61 |
| low dose 3e12 vg/kg | | | |

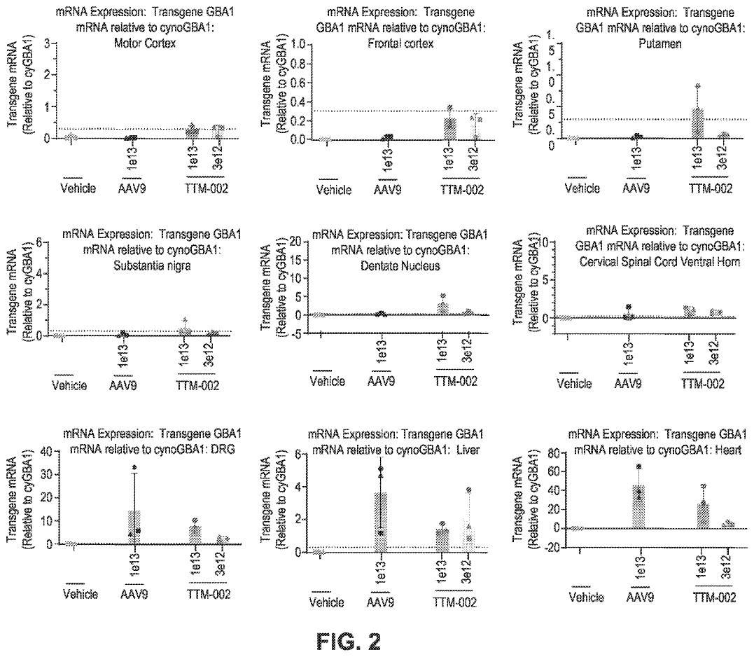

As compared to AAV9.GBA_VG7-HA. TGTM 2.GBA_VG17-HA led to increased human GBA1 mRNA in the dentate nucleus, cervical spinal cord, frontal cortex, motor cortex, putamen, and substantia nigra (FIG. 2: Tables 48A-E). Compared to aminals treated with AAV9.GBA_VG17-HA, comparable levels of human GBA1 mRNA expression were observed in the DRG, liver, or heart of animals treated with TTM-002.GBA_VG17-HA relative to endogenous cynomolgus GBA1 (Tables 48F-H).

TABLE 48A
Human GBA1 mRNA expression relative to endogenous
cynomolgus GBA1 mRNA in the frontal cortex

| | FC rel | FC rel | Fold |
|---|---|---|---|
| | cyGBA | cyGBA | over |
| Treatment | individ | Group | AAV9 |
| Vehicle | BLQ | BLQ | |
| | BLQ | | |
| | BLQ | | |
| AAV9.GBA_VG17-HA | 0.03 | 0.02 | |
| 1e13 vg/kg | 0.03 | | |
| | 0.01 | | |
| TTM-002.GBA_VG17-HA | 0.14 | 0.22 | 9.74 |
| high dose 1e13 vg/kg | 0.34 | | |
| | 0.19 | | |
| TTM-002.GBA_VG17-HA | 0.02 | 0.15 | 6.64 |
| low dose 3e12 vg/kg | 0.21 | | |
| | 0.23 | | |

TABLE 48B
Human GBA1 mRNA expression relative to endogenous
cynomolgus GBA1 mRNA in the motor cortex

| | FC rel | FC rel | Fold |
|---|---|---|---|
| | cyGBA | cyGBA | over |
| Treatment | individ | Group | AAV9 |
| Vehicle | BLQ | BLQ | |
| | BLQ | | |
| | BLQ | | |
| AAV9.GBA_VG17-HA | 0.02 | 0.01 | |
| 1e13 vg/kg | 0.02 | | |
| | 0.01 | | |
| TTM-002.GBA_VG17-HA | 0.20 | 0.29 | 22.31 |
| high dose 1e13 vg/kg | 0.23 | | |
| | 0.43 | | |
| TTM-002.GBA_VG17-HA | 0.05 | 0.25 | 19.08 |
| low dose 3e12 vg/kg | 0.34 | | |
| | 0.35 | | |

TABLE 48C
Human GBA1 mRNA expression relative to endogenous
cynomolgus GBA1 mRNA in the putamen

| | FC rel | FC rel | Fold |
|---|---|---|---|
| | cyGBA | cyGBA | over |
| Treatment | individ | Group | AAV9 |
| Vehicle | BLQ | BLQ | |
| | BLQ | | |
| | BLQ | | |
| AAV9.GBA_VG17-HA | 0.02 | 0.02 | |
| 1e13 vg/kg | 0.05 | | |
| | 0.00 | | |
| TTM-002.GBA_VG17-HA | 0.10 | 0.47 | 19.52 |
| high dose 1e13 vg/kg | 0.83 | | |
| | 0.47 | | |
| TTM-002.GBA_VG17-HA | 0.02 | 0.05 | 2.11 |
| low dose 3e12 vg/kg | | | |

TABLE 48D
Human GBA1 mRNA expression relative to endogenous
cynomolgus GBA1 mRNA in the substantia nigra

| | FC rel | FC rel | Fold |
|---|---|---|---|
| | cyGBA | cyGBA | over |
| Treatment | individ | Group | AAV9 |
| Vehicle | BLQ | BLQ | |
| | BLQ | | |
| | BLQ | | |
| AAV9.GBA_VG17-HA | 0.06 | 0.10 | |
| 1e13 vg/kg | 0.22 | | |
| | 0.02 | | |
| TTM-002.GBA_VG17-HA | 0.24 | 0.53 | 5.23 |
| high dose 1e13 vg/kg | 0.26 | | |
| | 1.07 | | |
| TTM-002.GBA_VG17-HA | 0.09 | 0.16 | 1.61 |
| low dose 3e12 vg/kg | | | |

TABLE 48E
Human GBA1 mRNA expression relative to endogenous
cynomolgus GBA1 mRNA in the spinal cord

| | FC rel | FC rel | Fold |
|---|---|---|---|
| | cyGBA | cyGBA | over |
| Treatment | individ | Group | AAV9 |
| Vehicle | BLQ | BLQ | |
| | BLQ | | |
| | BLQ | | |
| AAV9.GBA_VG17-HA | 0.09 | 0.58 | |
| 1e13 vg/kg | 1.50 | | |
| | 0.15 | | |
| TTM-002.GBA_VG17-HA | 0.44 | 1.01 | 1.73 |
| high dose 1e13 vg/kg | 1.40 | | |
| | 1.17 | | |
| TTM-002.GBA_VG17-HA | 0.56 | 0.80 | 1.37 |
| low dose 3e12 vg/kg | | | |

TABLE 48F
Human GBA1 mRNA expression relative to endogenous
cynomolgus GBA1 mRNA in the DRG

| | FC rel | FC rel | Fold |
|---|---|---|---|
| | cyGBA | cyGBA | over |
| Treatment | individ | Group | AAV9 |
| Vehicle | BLQ | BLQ | |
| | BLQ | | |
| | BLQ | | |
| AAV9.GBA_VG17-HA | 33.15 | 14.51 | |
| 1e13 vg/kg | 5.88 | | |
| | 4.49 | | |
| TTM-002.GBA_VG17-HA | 7.17 | 7.66 | 0.53 |
| high dose 1e13 vg/kg | 10.38 | | |
| | 5.43 | | |
| TTM-002.GBA_VG17-HA | 2.40 | 2.16 | 0.15 |
| low dose 3e12 vg/kg | | | |

TABLE 48G
Human GBA1 mRNA expression relative to endogenous
cynomolgus GBA1 mRNA in the heart

| | FC rel | FC rel | Fold |
|---|---|---|---|
| | cyGBA | cyGBA | over |
| Treatment | individ | Group | AAV9 |
| Vehicle | 0.00 | 0.04 | |
| | 0.00 | | |
| | 0.12 | | |
| AAV9.GBA_VG17-HA | 39.25 | 45.82 | |
| 1e13 vg/kg | 65.29 | | |
| | 32.92 | | |
| TTM-002.GBA_VG17-HA | 27.67 | 26.87 | 0.59 |
| high dose 1e13 vg/kg | 45.23 | | |
| | 7.71 | | |
| TTM-002.GBA_VG17-HA | 7.48 | 5.07 | 0.11 |
| low dose 3e12 vg/kg | | | |

TABLE 48H
Human GBA1 mRNA expression relative to endogenous
cynomolgus GBA1 mRNA in the liver

| | FC rel | FC rel | Fold |
|---|---|---|---|
| | cyGBA | cyGBA | over |
| Treatment | individ | Group | AAV9 |
| Vehicle | 0.00 | 0.00 | |
| | 0.00 | | |
| | 0.00 | | |
| AAV9.GBA_VG17-HA | 5.11 | 3.66 | |
| 1e13 vg/kg | 1.17 | | |
| | 4.70 | | |
| TTM-002.GBA_VG17-HA | 1.77 | 1.43 | 0.39 |
| high dose 1e13 vg/kg | 1.17 | | |
| | 1.35 | | |
| TTM-002.GBA_VG17-HA | 3.84 | 2.10 | 0.57 |
| low dose 3e12 vg/kg | | | |

Immunohistochemical analysis showed that TTM-002.GBA_VG17-HA led to greater GBA1-HA levels in the brain as compared to AAV9 (Table 49).

TABLE 49
HA chromogenic IHC quantitative image analysis following
TTM002.GBA_VG17-HA or AAV9.GBA_VG17-HA treatment

| | % HA + total cells per region | | | |
|---|---|---|---|---|
| | Frontal | Motor | | Substantia |
| Treatment | Cortex | Cortex | Putamen | Nigra |
| Vehicle | 0.2 | 0 | 0.5 | 0.2 |
| AAV9.GBA_VG17- | 0.5 | 0.1 | 0.6 | 1.3 |
| HA 1E13 vg/kg | | | | |
| TTM002.GBA_VG17- | 1.6 | 0.8 | 11.3 | 2.5 |
| HA 1E13 vg/kg | | | | |
| TTM002.GBA_VG17- | 0.9 | 0.5 | 2.4 | 0.4 |
| HA 3E12 vg/kg | | | | |

Co-staining analysis showed that HA protein expression was detected in both neurons and astrocytes of the putamen and substantia nigra of subjects treated with TTM002.GBA_VG 17-HA but not in subjects treated with AAV9.GBA_VG17-HA or vehicle (Table 50).

TABLE 50
Astrocyte co-staining analysis following
TTM002.GBA_VG17-HA or AAV9.GBA_VG17-HA treatment

| | | Putamen | | Substantia Nigra | |
|---|---|---|---|---|---|
| Animal | | | Astro- | | Astro- |
| ID | Treatment | Neurons | cytes | Neurons | cytes |
| 1 | Vehicle | − | − | − | − |
| 2 | AAV9.GBA_VG17- | + | − | + | − |
| | HA 1E13 vg/kg | | | | |
| 3 | TTM002.GBA_VG17- | TBD | TBD | + | + |
| | HA 1E13 vg/kg | | | | |
| 4 | TTM002.GBA_VG17- | + | + | + | + |
| | HA 1E13 vg/kg | | | | |
| 5 | TTM002.GBA_VG17- | + | + | TBD | TBD |
| | HA 1E13 vg/kg | | | | |

### IX. Equivalents and Scope

Those skilled in the art will recognize, or be able to ascertain using no more than routine experimentation, many equivalents to the specific embodiments in accordance with the Detailed Description provided herein. The scope of the present disclosure is not intended to be limited to the above Detailed Description, but rather is as set forth in the appended claims.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any, composition, therapeutic or active ingredient: any method of production: any method of use: etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. An adeno-associated virus (AAV) particle comprising:
a) an AAV capsid variant comprising an amino acid sequence having the following formula: [N1]-[N2]-[N3], wherein:
(i) optionally [N1] comprises X1, X2, and X3, wherein at least one of X1, X2, or X3 is G;
(ii) [N2] comprises the amino acid sequence of SPH; and
(iii) [N3] comprises X4, X5, and X6, wherein at least one of X4, X5, or X6 is a basic amino acid;
and
b) a viral genome comprising a syntaxin-binding protein 1 (STXBP1)-encoding sequence.

2. The AAV particle of claim 1, wherein the amino acid sequence [N1]-[N2]-[N3] is in hypervariable loop IV of the AAV capsid variant.

3. The AAV particle of claim 1 or claim 2, wherein the AAV capsid variant is an AAV9 capsid variant.

4. The AAV particle of any one of claims 1-3, wherein [N1] comprises X1, X2, and X3, wherein at least one of X1, X2, or X3 is G.

5. The AAV particle of any one of claims 1-4, wherein [N2]-[N3] comprises the amino acid sequence of SPHSKA (SEQ ID NO: 941).

6. An adeno-associated virus (AAV) particle comprising a viral genome comprising a syntaxin-binding protein 1 (STXBP1)-encoding sequence and an AAV9 capsid variant comprising the amino acid sequence of SPHSKA (SEQ ID NO: 941).

7. The AAV particle of claim 6, wherein the amino acid sequence of SPHSKA (SEQ ID NO: 941) is in hypervariable loop IV of the AAV9 capsid variant.

8. The AAV particle of claim 6 or claim 7, wherein the amino acid sequence of SPHSKA (SEQ ID NO: 941) is present immediately subsequent to an amino acid position corresponding to position 455 of SEQ ID NO: 4 or SEQ ID NO: 36.

9. The AAV particle of any one of claims 6-8, wherein the AAV9 capsid variant further comprises one, two, or all of: an N at an amino acid position corresponding to position 452, an E at an amino acid position corresponding to position 451, and/or a V at an amino acid position corresponding to position 453 of SEQ ID NO: 4.

10. The AAV particle of claim any one of claims 6-9, wherein the AAV9 capsid variant comprises the amino acid sequence of KTENVSGSPHSKAQNQQT (SEQ ID NO: 3272).

11. The AAV particle of any one of claims 6-10, wherein the AAV9 capsid variant comprises:
(i) a VP1 protein comprising an amino acid sequence having at least 90% identity to SEQ ID NO: 4;
(ii) a VP2 protein comprising an amino acid sequence having at least 90% identity to positions 138-742 of SEQ ID NO: 4; and/or
(iii) a VP3 protein comprising an amino acid sequence having at least 90% identity to positions 203-742 of SEQ ID NO: 4.

12. The AAV particle of any one of claims 6-11, wherein the AAV9 capsid variant comprises:
(i) a VP1 protein comprising an amino acid sequence having at least 95% identity to SEQ ID NO: 4;
(ii) a VP2 protein comprising an amino acid sequence having at least 95% identity to positions 138-742 SEQ ID NO: 4; and/or
(iii) a VP3 protein comprising an amino acid sequence having at least 95% identity to positions 203-742 of SEQ ID NO: 4.

13. The AAV particle of any one of claims 6-12, wherein the AAV9 capsid variant comprises:
(i) a VP1 protein comprising an amino acid sequence having at least 99% identity to SEQ ID NO: 4;
(ii) a VP2 protein comprising an amino acid sequence having at least 99% identity to positions 138-742 of SEQ ID NO: 4; and/or
(iii) a VP3 protein comprising an amino acid sequence having at least 99% identity to positions 203-742 of SEQ ID NO: 4.

14. The AAV particle of any one of claims 6-13, wherein the AAV9 capsid variant comprises:
(i) a VP1 protein comprising the amino acid sequence of SEQ ID NO: 4;
(ii) a VP2 protein comprising the amino acid sequence of positions 138-742 of SEQ ID NO: 4; and/or
(iii) a VP3 protein comprising the amino acid sequence of positions 203-742 of SEQ ID NO: 4.

15. The AAV particle of any one of claims 6-13, wherein the AAV9 capsid variant comprises:
(i) the amino acid sequence of SPHSKA (SEQ ID NO: 941), wherein the amino acid sequence is present immediately subsequent to an amino acid position corresponding to position 455 of SEQ ID NO: 4;
(ii) an E at an amino acid position corresponding to position 451 and a V at an amino acid position corresponding to position 453 of SEQ ID NO: 4; and
(iii) no other modifications relative to wild type AAV9.

16. The AAV particle of any one of claims 6-8, wherein the AAV9 capsid variant further comprises one, two, or all of: an E at an amino acid position corresponding to position 451, an R at an amino acid position corresponding to position 452, and/or a V at an amino acid position corresponding to position 453 of SEQ ID NO: 36.

17. The AAV particle of any one of claims 6-8 and 16, wherein the AAV9 capsid variant comprises the amino acid sequence of KTERVSGSPHSKAQNQQT (SEQ ID NO: 3589).

18. The AAV particle of any one of claims 6-8, 16, and 17, wherein the AAV9 capsid variant comprises:
(i) a VP1 protein comprising an amino acid sequence having at least 90% identity to SEQ ID NO: 36;
(ii) a VP2 protein comprising an amino acid sequence having at least 90% identity to positions 138-742 SEQ ID NO: 36; and/or
(iii) a VP3 protein comprising an amino acid sequence having at least 90% identity to positions 203-742 of SEQ ID NO: 36.

19. The AAV particle of any one of claims 6-8 and 16-18, wherein the AAV9 capsid variant comprises:
(i) a VP1 protein comprising an amino acid sequence having at least 95% identity to SEQ ID NO: 36;
(ii) a VP2 protein comprising an amino acid sequence having at least 95% identity to positions 138-742 SEQ ID NO: 36; and/or
(iii) a VP3 protein comprising an amino acid sequence having at least 95% identity to positions 203-742 of SEQ ID NO: 36.

20. The AAV particle of any one of claims 6-8 and 16-19, wherein the AAV9 capsid variant comprises:
(i) a VP1 protein comprising an amino acid sequence having at least 99% identity to SEQ ID NO: 36;
(ii) a VP2 protein comprising an amino acid sequence having at least 99% identity to positions 138-742 of SEQ ID NO: 36; and/or
(iii) a VP3 protein comprising an amino acid sequence having at least 99% identity to positions 203-742 of SEQ ID NO: 36.

21. The AAV particle of any one of claims 6-8 and 16-20, wherein the AAV9 capsid variant comprises:
(i) a VP1 protein comprising the amino acid sequence of SEQ ID NO: 36;
(ii) a VP2 protein comprising the amino acid sequence of positions 138-742 of SEQ ID NO: 36; and/or
(iii) a VP3 protein comprising the amino acid sequence of positions 203-742 of SEQ ID NO: 36.

22. The AAV particle of any one of claims 6-8 and 16-20, wherein the AAV9 capsid variant comprises:
(i) the amino acid sequence SPHSKA (SEQ ID NO: 941), wherein the amino acid sequence is present immediately subsequent to an amino acid position corresponding to position 455 of SEQ ID NO: 36;
(ii) an E at an amino acid position corresponding to position 451, an R at an amino acid position corresponding to position 452, and a V at an amino acid position corresponding to position 453 of SEQ ID NO: 36; and
(iii) no other modifications relative to wild type AAV9.

23. The AAV particle of any one of claims 1-4, wherein [N1]-[N2]-[N3] is present immediately subsequent to a position corresponding to the amino acid position 452 of SEQ ID NO: 982; and
wherein the AAV capsid variant comprises an amino acid sequence at least 90% identical, e.g., at least 91%, at least 92%, at least 93%, at least 94%, at least 95%, at least 96%, at least 97%, at least 98%, at least 99%, or 100% identical, to the amino acid sequence of SEQ ID NO: 982, e.g., to positions 203-742 of SEQ ID NO: 982.

24. The AAV particle of claim 23, wherein [N1] comprises GHD.

25. The AAV particle of claim 23 or claim 24, wherein [N1] comprises the amino acid G at a position corresponding to position 453, the amino acid H at position 454, and the amino acid D at position 455 of SEQ ID NO: 138 or SEQ ID NO: 982.

26. The AAV particle of any one of claims 23-25, wherein [N3] comprises KSG.

27. The AAV particle of any one of claims 23-26, wherein the AAV capsid variant comprises:
(i) a VP1 protein comprising the amino acid sequence of SEQ ID NO: 982 or an amino acid sequence having at least 90% identity to SEQ ID NO: 982;
(ii) a VP2 protein comprising the amino acid sequence of positions 138-742 of SEQ ID NO: 982 or an amino acid sequence having at least 90% identity to positions 138-742 SEQ ID NO: 982; or
(iii) a VP3 protein comprising the amino acid sequence of positions 203-742 of SEQ ID NO: 982 or an amino acid sequence having at least 90% identity to positions 203-742 of SEQ ID NO: 982.

28. The AAV particle of any one of claims 23-27, wherein the AAV capsid variant comprises:
(i) a VP1 protein comprising the amino acid sequence of SEQ ID NO: 982 or an amino acid sequence having at least 95% identity to SEQ ID NO: 982;
(ii) a VP2 protein comprising the amino acid sequence of positions 138-742 of SEQ ID NO: 982 or an amino acid sequence having at least 95% identity to positions 138-742 SEQ ID NO: 982: or
(iii) a VP3 protein comprising the amino acid sequence of positions 203-742 of SEQ ID NO: 982 or an amino acid sequence having at least 95% identity to positions 203-742 of SEQ ID NO: 982.

29. The AAV particle of any one of claims 23-28, wherein the AAV capsid variant comprises:
(i) a VP1 protein comprising the amino acid sequence of SEQ ID NO: 982 or an amino acid sequence having at least 99% identity to SEQ ID NO: 982;
(ii) a VP2 protein comprising the amino acid sequence of positions 138-742 of SEQ ID NO: 982 or an amino acid sequence having at least 99% identity to positions 138-742 SEQ ID NO: 982; or
(iii) a VP3 protein comprising the amino acid sequence of positions 203-742 of SEQ ID NO: 982 or an amino acid sequence having at least 99% identity to positions 203-742 of SEQ ID NO: 982.

30. The AAV particle of any one of claims 23-29, wherein the AAV capsid variant comprises:
(i) a VP1 protein comprising the amino acid sequence of SEQ ID NO: 982;
(ii) a VP2 protein comprising the amino acid sequence of positions 138-742 of SEQ ID NO: 982: or
(iii) a VP3 protein comprising the amino acid sequence of positions 203-742 of SEQ ID NO: 982.

31. The AAV particle of any one of claims 1-30, wherein the viral genome encodes a wildtype STXBP1 protein.

32. The AAV particle of any one of claims 1-31, wherein the viral genome encodes a human STXBP1 protein.

33. The AAV particle of claim 31 or claim 32, wherein the STXBP1 protein comprises the amino acid sequence of SEQ ID NO: 6413.

34. The AAV particle of any one of claims 1-33, wherein the STXBP1-encoding sequence comprises a nucleotide sequence that is at least 90% identical (e.g., at least 90% at least 91%, at least 92%, at least 93%, at least 94%, at least 95%, at least 96%, at least 97%, at least 98%, at least 99%, or 100% identical) to SEQ ID NO: 6414.

35. The AAV particle of claim 34, wherein the STXBP1-encoding sequence comprises a nucleotide sequence that is at least 95% identical to SEQ ID NO: 6414.

36. The AAV particle of claim 35, wherein the STXBP1-encoding sequence comprises a nucleotide sequence that is at least 99% identical to SEQ ID NO: 6414.

37. The AAV particle of claim 36, wherein the STXBP1-encoding sequence comprises the nucleotide sequence of SEQ ID NO: 6414.

38. The AAV particle of claim 36, wherein the STXBP1-encoding sequence consists of the nucleotide sequence of SEQ ID NO: 6414.

39. The AAV particle of any one of claims 1-38, wherein the viral genome comprises a promoter operably linked to the STXBP1-encoding sequence.

40. The AAV particle of claim 39, wherein the promoter is human elongation factor 1α-subunit (EF1α), cytomegalovirus (CMV) immediate-early enhancer and/or promoter, chicken β-actin (CBA), CAG, CAG derivative, β glucuronidase (GUSB), or ubiquitin C (UBC), neuron-specific enolase (NSE), platelet-derived growth factor (PDGF), platelet-derived growth factor B-chain (PDGF-β), intercellular adhesion molecule 2 (ICAM-2), synapsin (Syn), synapsin 1 (Syn1), methyl-CpG binding protein 2 (MeCP2), Ca2+/calmodulin-dependent protein kinase II (CaMKII), metabotropic glutamate receptor 2 (mGluR2), neurofilament light (NFL) or heavy (NFH), β-globin minigene nβ2, preproenkephalin (PPE), enkephalin (Enk) and excitatory amino acid transporter 2 (EAAT2), glial fibrillary acidic protein (GFAP), myelin basic protein (MBP), a cardiovascular promoter (e.g., αMHC, cTnT, and CMV-MLC2k), a liver promoter (e.g., hAAT, TBG), a skeletal muscle promoter (e.g., desmin, MCK, C512) or a fragment, e.g., a truncation, or a functional variant thereof.

41. The AAV particle of any one of claims 1-40, wherein the viral genome further comprises an inverted terminal repeat (ITR) sequence.

42. The AAV particle of claim 41, wherein the viral genome comprises an ITR sequence positioned 5′ relative to the STXBP1-encoding sequence.

43. The AAV particle of claim 41 or claim 42, wherein the viral genome comprises an ITR sequence positioned 3′ relative to the STXBP1-encoding sequence.

44. The AAV particle of any of claims 41-43, wherein the viral genome comprises an ITR sequence positioned 5′ relative to the STXBP1-encoding sequence and an ITR sequence positioned 3′ relative to the STXBP1-encoding sequence.

45. An adeno-associated virus (AAV) particle comprising a viral genome comprising a syntaxin-binding protein 1 (STXBP1)-encoding sequence and an AAV capsid variant comprising:
(i) a VP1 protein comprising the amino acid sequence of SEQ ID NO: 4;
(ii) a VP2 protein comprising the amino acid sequence of positions 138-742 of SEQ ID NO: 4; and/or
(iii) a VP3 protein comprising the amino acid sequence of positions 203-742 of SEQ ID NO: 4.

46. An adeno-associated virus (AAV) particle comprising a viral genome comprising syntaxin-binding protein 1 (STXBP1)-encoding sequence and an AAV capsid variant comprising:
(i) a VP1 protein comprising the amino acid sequence of SEQ ID NO: 36;
(ii) a VP2 protein comprising the amino acid sequence of positions 138-742 of SEQ ID NO: 36; and/or
(iii) a VP3 protein comprising the amino acid sequence of positions 203-742 of SEQ ID NO: 36.

47. A cell comprising the AAV particle of am one of claims 1-46, optionally wherein the cell is a mammalian cell (e.g., an HEK293 cell), an insect cell (e.g., an Sf9 cell), or a bacterial cell.

48. A method of making the AAV particle of any one of claims 1-46, the method comprising:
(i) providing a cell comprising the viral genome comprising a STXBP1-encoding sequence and a nucleic acid encoding the AAV capsid variant; and
(ii) incubating the cell under conditions suitable to encapsulate the viral genomic in the AAV capsid variant;
thereby making the AAV particle.

49. The method of claim 48, wherein:
(a) the viral genome comprises the nucleotide sequence of SEQ ID NO: 6414 or a nucleotide sequence that is at least 90% identical (e.g., at least 90%, at least 91%, at least 92%, at least 93%, at least 94%, at least 95%, at least 96%, at least 97%, at least 98%, or at least 99% identical) thereto; and
(b) the AAV capsid variant comprises:
(i) a VP1 protein comprising the amino acid sequence of SEQ ID NO: 4 or an amino acid sequence having at least 90% identity (e.g., at least 90%, at least 91%, at least 92%, at least 93%, at least 94%, at least 95%, at least 96%, at least 97%, at least 98%, or at least 99% identity) to SEQ ID NO: 4;
(ii) a VP2 protein comprising the amino acid sequence of positions 138-742 of SEQ ID NO: 4 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 138-742 SEQ ID NO: 4: or
(iii) a VP3 protein comprising the amino acid sequence of positions 203-742 of SEQ ID NO: 4 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 203-742 of SEQ ID NO: 4.

50. The method of claim 49, wherein the AAV capsid variant comprises the amino acid sequence of SEQ ID NO: 4, the amino acid sequence of positions 138-742 of SEQ ID NO: 4, and/or the amino acid sequence of positions 203-742 of SEQ ID NO: 4.

51. The method of claim 48, wherein:
(a) the viral genome comprises the nucleotide sequence of SEQ ID NO: 6414 or a nucleotide sequence that is at least 90% identical (e.g., at least 90%, at least 91%, at least 92%, at least 93%, at least 94%, at least 95%, at least 96%, at least 97%, at least 98%, or at least 99% identical) thereto; and
(b) the AAV capsid variant comprises:
(i) a VP1 protein comprising the amino acid sequence of SEQ ID NO: 36 or an amino acid sequence having at least 90% identity (e.g., at least 90%, at least 91%, at least 92%, at least 93%, at least 94%, at least 95%, at least 96%, at least 97%, at least 98%, or at least 99% identity) to SEQ ID NO: 36;
(ii) a VP2 protein comprising the amino acid sequence of positions 138-742 of SEQ ID NO: 36 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 138-742 SEQ ID NO: 36: or
(iii) a VP3 protein comprising the amino acid sequence of positions 203-742 of SEQ ID NO: 36 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 203-742 of SEQ ID NO: 36.

52. The method of claim 51, wherein the AAV capsid variant comprises the amino acid sequence of SEQ ID NO: 36, the amino acid sequence of positions 138-742 of SEQ ID NO: 36, and/or the amino acid sequence of positions 203-742 of SEQ ID NO: 36.

53. The method of claim 48, wherein:
(a) the viral genome comprises the nucleotide sequence of SEQ ID NO: 6414 or a nucleotide sequence that is at least 90% identical (e.g., at least 90%, at least 91%, at least 92%, at least 93%, at least 94%, at least 95%, at least 96%, at least 97%, at least 98%, or at least 99% identical) thereto; and
(b) the AAV capsid variant comprises:
(i) a VP1 protein comprising the amino acid sequence of SEQ ID NO: 982 or an amino acid sequence having at least 90% identity (e.g., at least 90%, at least 91%, at least 92%, at least 93%, at least 94%, at least 95%, at least 96%, at least 97%, at least 98%, or at least 99% identity) to SEQ ID NO: 982;
(ii) a VP2 protein comprising the amino acid sequence of positions 138-742 of SEQ ID NO: 982 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 138-742 SEQ ID NO: 982; or
(iii) a VP3 protein comprising the amino acid sequence of positions 203-742 of SEQ ID NO: 982 or an amino acid sequence having at least 90% identity (e.g., at least 90%, at least 91%, at least 92%, at least 93%, at least 94%, at least 95%, at least 96%, at least 97%, at least 98%, or at least 99% identity) to positions 203-742 of SEQ ID NO: 982.

54. The method of claim 53, wherein the AAV capsid variant comprises the amino acid sequence of SEQ ID NO: 982, the amino acid sequence of positions 138-742 of SEQ ID NO: 982, and/or the amino acid sequence of positions 203-742 of SEQ ID NO: 982.

55. The method of any one of claims 48-54, further comprising, prior to step (i), introducing a first nucleic acid molecule comprising the viral genome into the cell.

56. The method of any one of claims 48-55, wherein the cell comprises a second nucleic acid molecule encoding the AAV capsid variant, optionally wherein the method further comprises, prior to step (i), introducing the second nucleic acid molecule into the cell.

57. The method of any one of claims 48-56, wherein the cell comprises a mammalian cell (e.g., an HEK293 cell), an insect cell (e.g., an Sf9 cell), or a bacterial cell.

58. A pharmaceutical composition comprising the AAV particle of any one of claims 1-46, and a pharmaceutically acceptable excipient.

59. A pharmaceutical composition comprising the AAV particle of any one of claims 5-22, and a pharmaceutically acceptable excipient.

60. A pharmaceutical composition comprising the AAV particle of any one of claims 9-15 and 45, and a pharmaceutically acceptable excipient.

61. A pharmaceutical composition comprising the AAV particle of any one of claims 16-22 and 46, and a pharmaceutically acceptable excipient.

62. A method of delivering an AAV particle encoding an STXBP1 protein to a subject, comprising administering to the subject an effective amount of the pharmaceutical composition of any one of claims 58-61 or the AAV particle of any one of claims 1-46.

63. The method of claim 62, wherein the subject has, has been diagnosed with having, or is at risk of having a STXBP1-related disorder, optionally wherein the STXBP1-related disorder is a STXBP1-related neurodegenerative or neuromuscular disorder.

64. The method of claim 62 or claim 63, wherein the subject has, has been diagnosed with having, or is at risk of having STXBP1 encephalopathy, epileptic encephalopathy, Ohtahara syndrome, developmental encephalopathy, West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5).

65. A method of treating a subject having or diagnosed with having an STXBP1-related disorder, comprising administering to the subject an effective amount of the pharmaceutical composition of any one of claims 58-61 or the AAV particle of any one of claims 1-46.

66. A method of treating a subject having or diagnosed with having an STXBP1-related disorder, comprising administering to the subject an effective amount of the pharmaceutical composition of claim 59 or the AAV particle of any one of claims 5-22.

67. A method of treating a subject having or diagnosed with having an STXBP1-related disorder, comprising administering to the subject an effective amount of the pharmaceutical composition of claim 60 or the AAV particle of any one of claims 9-15 and 45.

68. A method of treating a subject having or diagnosed with having an STXBP1-related disorder, comprising administering to the subject an effective amount of the pharmaceutical composition of claim 61 or the AAV particle of any one of claims 16-22 and 46.

69. The method of any one of claims 65-68, wherein the STXBP1-related disorder is an STXBP1-related neurodegenerative or neuromuscular disorder.

70. The method of claim 69, wherein the STXBP1-related neurodegenerative or neuromuscular disorder is STXBP1 encephalopathy, epileptic encephalopathy, Ohtahara syndrome, developmental encephalopathy, West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5).

71. A method of treating a subject having STXBP1 encephalopathy or diagnosed with having STXBP1 encephalopathy, comprising administering to the subject an effective amount of the pharmaceutical composition of any one of claims 58-61 or the AAV particle of any one of claims 1-46.

72. A method of treating a subject having STXBP1 encephalopathy or diagnosed with having STXBP1 encephalopathy, comprising administering to the subject an effective amount of pharmaceutical composition of claim 59 or the AAV particle of any one of claims 5-22.

73. A method of treating a subject having STXBP1 encephalopathy or diagnosed with having STXBP1 encephalopathy, comprising administering to the subject an effective amount of the pharmaceutical composition of claim 60 or the AAV particle of any one of claims 9-15 and 45.

74. A method of treating a subject having STXBP1 encephalopathy or diagnosed with having STXBP1 encephalopathy, comprising administering to the subject an effective amount of the pharmaceutical composition of claim 61 or the AAV particle of any one of claims 16-22 and 46.

75. The method of any one of claims 62-74, wherein the subject has one or more mutations in the STXBP1 gene.

76. The method of any one of claims 62-75, wherein the subject has a reduced level of STXBP1 activity as compared to a reference level in a subject who does not have an STXBP1-related disorder.

77. The method of any one of claims 65-76, wherein the treating results in prevention of progression of the disorder in the subject.

78. The method of any one of claims 65-77, wherein the treating results in amelioration of at least one symptom of the disorder and/or a change in one or more biomarkers of the disorder.

79. The method of claim 78, wherein the one or more biomarkers comprises an STXBP1 activity or neurofilament light chain.

80. The method of claim 78, wherein the at least one symptom comprises epilepsy, autistic features, ataxia, generalized tremors, dystonia, or a combination thereof.

81. The method of any one of claims 62-80, wherein the subject is a human.

82. The method of any one of claims 62-81, wherein the AAV particle is delivered to a cell, tissue, or region of the CNS, e.g., a region of the brain or spinal cord, e.g., the parenchyma, the cortex, substantia nigra, caudate cerebellum, striatum, corpus callosum, cerebellum, brain stem caudate-putamen, thalamus, superior colliculus, the spinal cord, or a combination thereof, and/or to neurons, e.g. GABAergic neurons, glutamatergic neurons, or a combination thereof.

83. The method of any one of claims 62-82, further comprising evaluating, e.g., measuring, the level of STXBP1 expression, e.g., STXBP1 gene expression, STXBP1 mRNA expression, and/or STXBP1 protein expression, in the subject, e.g., in a cell, tissue, or fluid of the subject.

84. The method of claim 83, wherein the level of STXBP1 protein expression is measured by an ELISA, a Western blot, or an immunohistochemistry assay.

85. The method of claim 83 or claim 84, wherein evaluating the level of STXBP1 expression is performed prior to and subsequent to administration of the AAV particle, optionally wherein the level of STXBP1 expression prior to treatment is compared to the level of STXBP1 expression subsequent to administration.

86. The method of any one of claims 83-85, comprising evaluating the level of STXBP1 expression in a cell or tissue of the central nervous system (e.g., parenchyma).

87. The method of any one of claims 83-86, wherein the subject's level of STXBP1 protein expression subsequent to administration is increased relative to the subject's level of STXBP1 protein expression prior to administration.

88. The method of any one of claims 62-87, further comprising evaluating, e.g., measuring, the level of STXBP1 activity in the subject, e.g., in a cell or tissue of the subject.

89. The method of any one of claims 62-88, wherein the administration results in an increase in:
(i) STXBP1 activity in a cell, tissue, (e.g., a cell or tissue of the CNS, e.g., the cortex, striatum, thalamus, cerebellum, and/or brainstem), and/or fluid (e.g., CSF and/or serum) of the subject, relative to STXBP1 activity in the subject prior to the administration,
(ii) viral genomes (VG) per cell level in a CNS tissue (e.g., the cortex, striatum, thalamus, cerebellum, brainstem, and/or spinal cord) of the subject, relative to the subject's VG per cell level in a peripheral tissue; and/or
(iii) STXBP1 mRNA expression in a cell or tissue (e.g., a cell or tissue of the CNS, e.g., the cortex, thalamus, and/or brainstem) of the subject, as compared to STXBP1 mRNA expression in the subject prior to the administration.

90. The method of any one of claims 65-89, further comprising administering to the subject an additional agent suitable for treatment or prevention of an STXBP1-related disorder,
optionally wherein the additional agent comprises one or more anti-epileptic drugs (e.g., levetiracetam, phenobarbital, clobazam, topiramate), adrenocorticotropic hormone, or a combination thereof.

91. The method of any one of claims 65-90, further comprising administering an immunosuppressant to the subject.

92. The method of claim 91, wherein the immunosuppressant comprises a corticosteroid (e.g., prednisone, prednisolone, methylprednisolone, and/or dexamethasone), rapamycin, mycophenolate mofetil, tacrolimus, rituximab, and/or eculizumab hydroxychloroquine.

93. The pharmaceutical composition of any one of claims 58-41 or the AAV particle of any one of claims 1-46, for use in the treatment of an STXBP1-related disorder, optionally wherein the STXBP1-related disorder is STXBP1 encephalopathy, epileptic encephalopathy, Ohtahara syndrome, developmental encephalopathy, West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5).

94. Use of the pharmaceutical composition of any one of claims 58-61 or the AAV particle of any one of claims 1-46 in the manufacture of a medicament for the treatment of an STXBP1-related disorder, optionally wherein the STXBP1-related disorder is STXBP1 encephalopathy, epileptic encephalopathy, Ohtahara syndrome, developmental encephalopathy, West syndrome, early myoclonic epileptic encephalopathy, Lennox-Gaustaut syndrome, autism (e.g., autism with STXBP1 mutations and optionally further mutations), Dravet syndrome (not caused by mutations in SCN1A), or Rett syndrome phenotype (not caused by mutation of MECP2 or CDKL5).
